(19) United States
(12) Patent Application Publication
(10) Pub. No.: US 2022/0162285 A1
(43) Pub. Date: May 26, 2022

(54) MULTIMERS

(71) Applicant: Quadrucept Bio Limited, London (GB)

(72) Inventors: Hanif ALI, Cambridge (GB); Terence RABBITTS, Cambridge (GB); Jasper CLUBE, London (GB); Christian GRØNDAHL, Cambridge (GB)

(21) Appl. No.: 17/440,604

(22) PCT Filed: Mar. 12, 2020
(86) PCT No.: PCT/EP2020/056737

§ 371 Date: Sep. 17, 2021

(30) Foreign Application Priority Data
Mar. 19, 2019 (GB) 1903767.0

(51) Int. Cl. C07K 14/705

(52) U.S. Cl. CPC C07K 14/705; C07K 2317/526; C07K 2319/30; C07K 2317/622; C07K 2317/524

(57) ABSTRACT

The invention relates to multimers such as tetramers of polypeptides; and tetramers, octamers, dodecamers and hexadecamers of epitopes or effector domains, such as antigen binding sites (eg, antibody or TCR binding sites that specifically bind to antigen or pMHC, or variable domains thereof) or peptides such as incretin, insulin or hormone peptides.

### TECHNICAL FIELD

The invention relates to multimers such as tetramers of polypeptides; and tetramers, octamers, dodecamers and hexadecamers of epitopes or effector domains, such as antigen binding sites (eg, antibody or TCR binding sites that specifically bind to antigen or pMHC, or variable domains thereof) or peptides such as incretin, insulin or hormone peptides.

### BACKGROUND

Multimers of effector domains have recognized utility in medical and non-medical applications for combining and multiplying the activity and presence of effector domains, eg, to provide for higher avidity of antigen binding (for effector domains that are antibody or TCR binding domains, for example) or for enhancing biological or binding activity, such as for providing bi- or multi-specific targeting or interaction with target ligands in vivo or in vitro.

Multimerisation domains which cause self-assembly of protein monomers into multimers are known in the art. Examples include domains found in transcription factors such as p53, p63 and p73, as well as domains found in ion channels such as TRP cation channels. The transcription factor p53 can be divided into different functional domains: an N-terminal transactivation domain, a proline-rich domain, a DNA-binding domain, a tetramerisation domain and a C-terminal regulatory region. The tetramerisation domain of human p53 extends from residues 325 to 356, and has a 4-helical bundle fold (Jeffrey et al., Science (New York, N.Y.) 1995, 267(5203):1498-1502). The TRPM tetramerisation domain is a short anti-parallel coiled-coil tetramerisation domain of the transient receptor potential cation channel subfamily M member proteins 1-8. It is held together by extensive core packing and interstrand polar interactions (Fujiwara et al., Journal of Molecular Biology 2008, 383(4):854-870). Transient receptor potential (TRP) channels comprise a large family of tetrameric cation-selective ion channels that respond to diverse forms of sensory input. Another example is the potassium channel BTB domain. This domain can be found at the N terminus of voltage-gated potassium channel proteins, where represents a cytoplasmic tetramerisation domain (Ti) involved in assembly of alpha-subunits into functional tetrameric channels (Bixby et al., Nature Structural Biology 1999, 6(1):38-43). This domain can also be found in proteins that are not potassium channels, like KCTD1 (potassium channel tetramerisation domain-containing protein 1; Ding et al., DNA and Cell Biology 2008, 27(5):257-265).

Multimeric antibody fragments have been produced using a variety of multimerisation techniques, including biotin, dHLX, ZIP and BAD domains, as well as p53 (Thie et al., Nature Boitech., 2009:26, 314-321). Biotin, which is efficient in production, is a bacterial protein which induces immune reactions in humans.

Human p53 (UniProtKB—P04637 (P53_HUMAN)) acts as a tumor suppressor in many tumor types, inducing growth arrest or apoptosis depending on the physiological circumstances and cell type. It is involved in cell cycle regulation as a trans-activator that acts to negatively regulate cell division by controlling a set of genes required for this process. Human p53 is found in increased amounts in a wide variety of transformed cells. It is frequently mutated or inactivated in about 60% of cancers. Human p53 defects are found in Barrett metaplasia a condition in which the normally stratified squamous epithelium of the lower esophagus is replaced by a metaplastic columnar epithelium. The condition develops as a complication in approximately 10% of patients with chronic gastroesophageal reflux disease and predisposes to the development of esophageal adenocarcinoma.

Nine isoforms of p53 naturally occur and are expressed in a wide range of normal tissues but in a tissue-dependent manner. Isoform 2 is expressed in most normal tissues but is not detected in brain, lung, prostate, muscle, fetal brain, spinal cord and fetal liver. Isoform 3 is expressed in most normal tissues but is not detected in lung, spleen, testis, fetal brain, spinal cord and fetal liver. Isoform 7 is expressed in most normal tissues but is not detected in prostate, uterus, skeletal muscle and breast. Isoform 8 is detected only in colon, bone marrow, testis, fetal brain and intestine. Isoform 9 is expressed in most normal tissues but is not detected in brain, heart, lung, fetal liver, salivary gland, breast or intestine.

### SUMMARY OF THE INVENTION

The invention provides: A polypeptide comprising an antibody Fc region, wherein the Fc region comprises an antibody CH2 and an antibody CH3; and a self-associating multimerisation domain (SAM); wherein the CH2 comprises an antibody hinge sequence and is devoid of a core hinge region. Advantageously, the Fc does not directly pair with another Fc, which is useful for producing multimers by multimerization using SAM domains. For example, a benefit may be aiding desired multimer formation and/or enhancing multimer purity formed by such multimerization.

The invention also provides: A multimer of a plurality of antibody Fc regions, wherein each Fc is comprised by a respective polypeptide and is unpaired with another Fc region; optionally wherein the multimer is for medical use.

The invention also provides:—

In a First Configuration

A protein multimer of at least first, second, third and fourth copies of an effector domain (eg, a protein domain or a peptide), wherein the multimer is multimerised by first, second, third and fourth self-associating tetramerisation domains (TDs) which are associated together, wherein each tetramerisation domain is comprised by a respective engineered polypeptide comprising one or more copies of said protein domain or peptide.

In a Second Configuration

An isolated tetramer, octamer, dodecamer, hexadecamer or 20-mer of a TCR binding site, insulin peptide, incretin peptide or peptide hormone; or a plurality of said tetramers or octamers.

An isolated tetramer, octamer, dodecamer, hexadecamer or 20-mer of an antibody binding site or an antibody variable domain (eg, a single variable domain); or a plurality of said tetramers or octamers.

In an example the tetramer or octamer is soluble in aqueous solution (eg, aqueous eukaryotic cell culture medium). In an example the tetramer or octamer is expressible in a eukaryotic cell. Exemplification is provided below.

In a Third Configuration

A tetramer, octamer, dodecamer, hexadecamer or 20-mer (eg, a tetramer or octamer) of

(a) TCR V domains or TCR binding sites, wherein the tetramer or octamer is soluble in aqueous solution (eg, an aqueous eukaryotic cell growth medium or buffer);
(b) antibody single variable domains, wherein the tetramer or octamer is soluble in aqueous solution (eg, an aqueous eukaryotic cell growth medium or buffer);
(c) TCR V domains or TCR binding sites, wherein the tetramer or octamer is capable of being intracellularly and/or extracellularly expressed by HEK293 cells; or
(d) antibody variable domains (eg, antibody single variable domains), wherein the tetramer or octamer is capable of being intracellularly and/or extracellularly expressed by HEK293 cells.

In a Fourth Configuration

An engineered polypeptide or monomer of a multimer, tetramer, octamer, dodecamer, hexadecamer or 20-mer (eg, a tetramer or octamer) of the invention.

In a Fifth Configuration

An engineered (and optionally isolated) engineered polypeptide (P1) which comprises (in N- to C-terminal direction):—

(a) TCR V1-TCR C1—antibody CH1 (eg, IgG CH1)—optional linker—TD, wherein

### (i) V1 is a Vα and C1 is a Cα;

### (ii) V1 is a Vβ and C1 is a Cβ;

(iii) V1 is a Vγ and C1 is a Cγ; or

### (iv) V1 is a Vδ and C1 is a Cδ;

or
(b) TCR V1—antibody CH1 (eg, IgG CH1)—optional linker—TD, wherein

### (i) V1 is a Vα;

### (ii) V1 is a Vβ;

(iii) V1 is a Vγ; or

### (iv) V1 is a Vδ;

or
(c) antibody V1—antibody CH1 (eg, IgG CH1)—optional linker—TD, wherein

### (i) V1 is a VH; or

### (ii) V1 is a VL (eg, a Vλ or a Vκ);

or
(d) antibody V1—optional antibody CH1 (eg, IgG CH1)—antibody Fc (eg, an IgG Fc)—optional linker—TD, wherein

### (i) V1 is a VH; or

### (ii) V1 is a VL (eg, a Vλ or a Vκ);

or
(e) antibody V1—antibody CL (eg, a Cλ or a Cκ)—optional linker—TD, wherein

### (i) V1 is a VH; or

### (ii) V1 is a VL (eg, a Vλ or a Vκ);

or
(f) TCR V1-TCR C1—optional linker—TD, wherein

### (i) V1 is a Vα and C1 is a Cα;

### (ii) V1 is a Vβ and C1 is a Cβ;

(iii) V1 is a Vγ and C1 is a Cγ; or

### (iv) V1 is a Vδ and C1 is a Cδ.

In a Sixth Configuration

A nucleic acid encoding an engineered polypeptide or monomer of the invention, optionally wherein the nucleic acid is comprised by an expression vector for expressing the polypeptide.

In a Seventh Configuration

Use of a nucleic acid or vector of the invention in a method of manufacture of protein multimers for producing intracellularly expressed and/or secreted multimers, wherein the method comprises expressing the multimers in and/or secreting the multimers from eukaryotic cells comprising the nucleic acid or vector.

In an Eighth Configuration

A method producing

(a) TCR V domain multimers, the method comprising the soluble and/or intracellular expression of TCR V-TD (eg, NHR2 TD or TCR V-p53 TD) fusion proteins expressed in eukaryotic cells, the method optionally comprising isolating a plurality of said multimers;
(b) antibody V domain multimers, the method comprising the soluble and/or intracellular expression of antibody V (eg, a single variable domain)-TD (eg, V-NHR2 TD or V-p53 TD) fusion proteins expressed in eukaryotic cells, the method optionally comprising isolating a plurality of said multimers;
(c) incretin peptide (eg, GLP-1, GIP or insulin) multimers, the method comprising the soluble and/or intracellular expression of incretin peptide-TD (eg, incretin peptide-NHR2 TD or incretin peptide-p53 TD) fusion proteins expressed in eukaryotic cells, such as HEK293T cells; the method optionally comprising isolating a plurality of said multimers; or
(d) peptide hormone multimers, the method comprising the soluble and/or intracellular expression of peptide hormone-TD (eg, peptide hormone-NHR2 TD or peptide hormone-p53 TD) fusion proteins expressed in eukaryotic cells, such as HEK293T cells; the method optionally comprising isolating a plurality of said multimers.

In a Ninth Configuration

Use of a nucleic acid or vector of the invention in a method of manufacture of protein multimers for producing glycosylated multimers in eukaryotic cells comprising the nucleic acid or vector.

In a Tenth Configuration

Use of self-associating tetramerisation domains (TD) (eg, NHR2 TD, p53 TD, p63 TD or p73 TD or a homologue or orthologue thereof) in a method of the manufacture of a tetramer of polypeptides, for producing a higher yield of tetramers versus monomer and/or dimer polypeptides.

In a Eleventh Configuration

Use of an engineered polypeptide in a method of the manufacture of a tetramer of a polypeptide comprising multiple copies of a protein domain or peptide, for producing a higher yield of tetramers versus monomer and/or dimer polypeptides, wherein the engineered polypeptide comprises one or more copies of said protein domain or peptide and further comprises a self-associating tetramerisation domains (TD) (eg, NHR2 TD, p53 TD, p63 TD or p73 TD or a homologue or orthologue).

In a Twelfth Configuration

Use of self-associating tetramerisation domains (TD) (eg, NHR2 TD, p53 TD, p63 TD or p73 TD or a homologue or orthologue thereof) in a method of the manufacture of a tetramer of a polypeptide, for producing a plurality of tetramers that are not in mixture with monomers, dimers or trimers.

In a Thirteenth Configuration

A eukaryotic host cell comprising the nucleic acid or vector for intracellular and/or secreted expression of the multimer, tetramer, octamer, dodecamer, hexadecamer or 20-mer (eg, tetramer, octamer), engineered polypeptide or monomer of the invention.

In a Fourteenth Configuration

Use of an engineered polypeptide in a method of the manufacture of a tetramer of a polypeptide comprising multiple copies of a protein domain or peptide, for producing a plurality of tetramers that are not in mixture with monomers, dimers or trimers, wherein the engineered polypeptide comprises one or more copies of said protein domain or peptide and further comprises a self-associating tetramerisation domains (TD) (eg, NHR2 TD, p53 TD, p63 TD or p73 TD or a homologue or orthologue).

In a Fifteenth Configuration

A multivalent heterodimeric soluble T cell receptor capable of binding pMHC complex comprising:

(i) TCR extracellular domains;
(ii) immunoglobulin constant domains; and
(iii) an NHR2 multimerisation domain of ETO.

In a Sixteenth Configuration

A multimeric immunoglobulin, comprising

(i) immunoglobulin variable domains; and
(ii) an NHR2 multimerisation domain of ETO.

In a Seventeenth Configuration

A method for assembling a soluble, multimeric polypeptide, comprising:

(a) providing a monomer of the said multimeric polypeptide, fused to an NHR2 domain of ETO;
(b) causing multiple copies of said monomer to associate, thereby obtaining a multimeric, soluble polypeptide.

In an Eighteenth Configuration

A mixture comprising (i) a cell line (eg, a eukaryotic, mammalian cell line, eg, a HEK293, CHO or Cos cell line) encoding a polypeptide of the invention; and (ii) tetramers of the invention.

In a Nineteenth Configuration

A method for enhancing the yield of tetramers of an protein effector domain (eg, an antibody variable domain or binding site), the method comprising expressing from a cell line (eg, a mammalian cell, CHO, HEK293 or Cos cell line) tetramers of a polypeptide, wherein the polypeptide is a polypeptide of the invention and comprises one or more effector domains; and optionally isolating said expressed tetramers.

In a Twentieth Configuration

A polypeptide comprising (in N- to C-terminal direction; or in C- to N-terminal direction)

(i) An immunoglobulin superfamily domain;
(ii) An optional linker; and
(iii) A self-associating multimerisation domain (SAM) (optionally a self-associating tetramerisation domain (TD)).

The invention also provides a pharmaceutical composition, cosmetic, foodstuff, beverage, cleaning product, detergent comprising the multimer(s), tetramer, octamer, dodecamer, hexadecamer or 20-mer (eg, tetramer(s) or octamer(s)) of the invention.

A multimer herein is, eg, a dimer, trimer, tetramer, octamer, dodecamer, hexadecamer or 20-mer.

As demonstrated in Example 22, dodecamer and hexadecamer multimers surprisingly display a very high functional affinity for antigen binding due to the increasing avidity effect. The functional affinity for these going from 8 to 12 binding sites (compare Tables 15 and 16) or from 8 to 16 binding sites is much more than additive; a synergistic increase is seen as a result of enhanced avidity. Thus, in one embodiment, a multimer which is 12-valent for an antigen (ie, a dodecamer as described herein) is preferred; in another embodiment a multimer which is 16-valent for an antigen (ie, hexadecamer as described herein) is preferred.

### BRIEF DESCRIPTION OF THE DRAWINGS

FIG. 1: A schematic drawing representing the stepwise self-assembly of a tetravalent heterodimeric soluble TCR protein complex via a monomer and homodimer, which is aided by NHR2 tetramerisation domain.

FIG. 2: A schematic drawing representing the stepwise self-assembly of an octavalent heterodimeric soluble TCR protein complex via a monomer² and homodimer2, which is aided by NHR2 tetramerisation domain and immunoglobulin hinge domain.

FIG. 3: A schematic drawing of the domain arrangements in the α and β chain used for expressing and assembling ts-NY-ESO-1 TCR.

FIG. 4: A schematic drawing of the domain arrangements in the α and β chain used for expressing and assembling os-NY-ESO-1 TCR.

FIG. 5: Amino acid sequence of the α and β chain of the ts-NY-ESO-1 TCR protein complex. Amino acid sequences of alternate domains are underlined.

FIG. 6: Amino acid sequence of the α and β chain of the os-NY-ESO-1 TCR protein complex. Amino acid sequences of alternate domains are underlined.

FIG. 7: A schematic drawing of the domain arrangements in the α and β chain used for expressing and assembling ts-NY-ESO-1 TCR-IL2 fusion protein complex.

FIG. 8: A schematic drawing of the domain arrangements in the α and β chain used for expressing and assembling os-NY-ESO-1 TCR-IL2 fusion protein complex.

FIG. 9: Amino acid sequence of the α and β chain of the ts-NY-ESO-1 TCR-IL2 fusion protein complex. Amino acid sequences of alternate domains are underlined.

FIG. 10: Amino acid sequence of the α and β chain of the os-NY-ESO-1 TCR-IL2 fusion protein complex. Amino acid sequences of alternate domains are underlined.

FIG. 11A: A schematic drawing representing the stepwise self-assembly of a tetravalent single domain antibody (dAb) complex via a monomer and homodimer, which is aided by NHR2 tetramerisation domain.

FIG. 11B: A schematic drawing of the domain arrangements for assembly of tetravalent dAbs, including linker and NHR2 domains.

FIG. 12A: A schematic drawing representing the stepwise self-assembly of a tetravalent Fab complex via a monomer and homodimer, which is aided by NHR2 tetramerisation domain.

FIG. 12B: A schematic drawing of the domain arrangements for assembly of tetravalent Fabs, including linker and NHR2 domains in the heavy chain, and light chain variable and constant domains.

FIG. 13A: A schematic drawing representing the stepwise self-assembly of an octavalent Fab complex via a monomer and homodimer, which is aided by NHR2 tetramerisation domain and an antibody hinge region linked to CH1 domain.

FIG. 13B: A schematic drawing of the domain arrangements for assembly of octavalent Fabs, including hinge, linker and NHR2 domains in the heavy chain, and light chain variable and constant domains.

FIG. 14: is a schematic of Quad 16 and Quad 17.

FIG. 15: shows (A) Quad 16 and (B) Quad 17 monomer sequences and configuration.

FIG. 16: shows analysis of secreted proteins using anti-Ig Western Blot: (A) a PAGE gel under SDS denatured conditions—16=Quad16; 17=Quad17; and (B) a PAGE gel under native (ie, non-denatured) conditions—16=Quad16; 17=Quad17.

FIG. 17: Western blots prepared from denaturing SDS-PAGE gel probed with anti-human IgG HRP detection antibody (A) Protein samples from Quads 3 and 4 were prepared from whole cell extracts and loaded in lanes 1 and 2 respectively. The expected Mw for Quads 3 and 4 are 46.1 and 46.4 kDa respectively. (B) Protein samples from Quads 12 and 13 were prepared from whole cell extracts and loaded in lanes 1 and 2 respectively. The expected Mw for Quads 12 and 13 are 47.8 and 48.1 kDa respectively.

FIG. 18: Western blots prepared from denaturing SDS-PAGE gel probed with anti-human IgG HRP detection antibody (A) Protein samples from Quads 3 and 4 were prepared by concentrating cell supernatant and loaded in lanes 1 and 2 respectively. The expected Mw for Quads 3 and 4 are 46.1 and 46.4 kDa respectively. (B) Protein samples from Quads 12 and 13 were prepared by concentrating cell supernatant and loaded in lanes 1 and 2 respectively. The expected Mw for Quads 12 and 13 are 47.8 and 48.1 kDa respectively.

FIG. 19: Western blots prepared from denaturing SDS-PAGE gel probed with anti-HIS HRP detection antibody (A) Protein samples from Quads 14, 15, 18 and 19 were prepared from whole cell extracts and loaded in lanes 1-4, respectively. The expected Mw for Quads 14, 15, 18 and 19 are 22.0, 22.3, 37.4 and 37.7 kDa respectively. (B) Protein samples from Quads 23, 24, 26 and 27 were prepared from whole cell extracts and loaded in lanes 1-4, respectively. The expected Mw for Quads 23, 24, 26 and 27 are 32.1, 32.4, 33.7 and 34.0 kDa respectively. (C) Protein samples from Quads 34, and 38 were prepared from whole cell extracts and loaded in lanes 1-2, respectively. The expected Mw for Quads 34, and 38 are 25.5 and 25.4 kDa respectively. (D) Protein samples from Quads 40, 42, 44 and 46 were prepared from whole cell extracts and loaded in lanes 1-4, respectively. The expected Mw for Quads 40, 42, 44 and 46 are 25.4, 37.6, 25.5 and 38.0 kDa respectively. Lane U contains concentrated serum prepared from untransfected HEK293T cells (negative control) and C is a His-tagged protein used as a positive control for the anti-His HRP detection antibody. Serum anti-His background band is highlighted by a black arrow, which can be consistently detected in all for blots.

FIG. 20: Western blot prepared from denaturing SDS-PAGE gel (A) and probed with anti-human IgG HRP detection antibody. Protein samples from Quads 14 and 15 were prepared from whole cell extracts and loaded in lanes 1 and 2, respectively. The expected Mw for Quads 14 and 15 are 22.0 and 22.3 kDa respectively. (B) Western blot prepared from Native PAGE gels and probed with anti-human IgG HRP detection antibody. Lanes 1 and 2 contains protein samples from Quads 14 and 15 prepared from whole cell extract.

FIG. 21: Quad polypeptides fused to leader and tag sequences. Where linker is present, the linker is G4S (only 1 G4S). * denotes TCR constant domains with introduced cysteine residue allowing S-S bond formation between TCR alpha and beta chain. Human IgG1 hinge was used. All C regions are human. The TCR V domains are specific for NY-ESO-1. GFP=green fluorescent protein.

FIG. 22: Schematic representations of the multimeric structure of Quad formats A-AC. The description and the monomeric building block from which the tetravalent Quad molecules are assembled from are described in Table 8.

FIG. 23: SDS-PAGE analysis of monospecific tetravalent dAb Quad 57 protein purified from culture supernatant. Quad protein migrated according to its expected MW as indicated by the arrow with no visible impurities.

FIG. 24: SDS-PAGE analysis of bispecific tetravalent dAb Quad 54 protein purified from culture supernatant (A). Quad protein migrated according to its expected MW as indicated by the arrow with no visible impurities (B) Direct binding ELISA using serially diluted Quad 54 protein with a fixed concentration of recombinant TNFa protein coated on plate. Quad 54 binds TNFa protein in a dose-dependent manner.

FIG. 25: SDS-PAGE analysis of monospecific tetravalent scFv Quads. Quads 51 and 63 proteins purified from culture supernatant and analysed by SDS-PAGE (A). Quad proteins migrated according to their expected MW as indicated by the arrows with no visible impurities (B) Direct binding ELISA using serially diluted Quad 51 and 63 proteins with a fixed concentration of recombinant TNFa protein coated on plate. Both Quads 51 and 63 bind TNFa protein with similar binding strength in a dose-dependent manner. (C) SDS-PAGE analysis of W51ScFv monovalent anti-TNFa control protein. (D) Western blot analysis of TNFa-mediated Caspase-3 signaling in the presence of Quad 51, Humira (Hum) and W51ScFv. Culture medium (CM) alone or with actinomycin D (AD) were used as a negative control. The detection antibody used for each blot is indicated next to each blots. The Western blots detected by anti-Tubulin represents internal loading control. (E) SDS-PAGE analysis of Quad 53 Tet protein purified from culture supernatant. The Quad protein migrated according to its expected MW as indicated by the arrow. (F). Direct binding ELISA using serially diluted Quad 53 Tet protein with a fixed concentration of recombinant CD20 protein coated on plate. Quad 53 Tet binds CD20 protein in a dose-dependent manner.

FIG. 26: SDS-PAGE analysis of monospecific octavalent scFv Quad 53 protein purified from culture supernatant (A). Quad protein migrated according to its expected MW as indicated by the arrow with no visible impurities (B) Direct binding ELISA using serially diluted Quad 53 Oct protein with a fixed concentration of recombinant CD20 protein coated on plate. Quad 53 Oct binds CD20 protein in a dose-dependent manner. (C) Monovalent, tetravalent and octavalent version of Quad 53 analysed by SDS-PAGE. (D) Direct binding ELISA comparing binding strength of monovalent, tetravalent and octavalent version of Quad 53 to recombinant CD20. An increasing in binding strength can be seen with increasing valency of Quad 53.

FIG. 27: SDS-PAGE analysis of bispecific tetravalent scFv Quad 55 protein purified from culture supernatant (A). Quad protein migrated according to its expected MW as indicated by the arrow with no visible impurities (B) Direct binding ELISA using serially diluted Quad 55 protein with a fixed concentration of recombinant TNFa protein coated on plate. Quad 55 binds TNFa protein in a dose-dependent manner.

FIG. 28: SDS-PAGE analysis of bispecific tetravalent Quads. Bispecific scFv×dAb Quad 56 protein purified from culture supernatant and analysed by SDS-PAGE (A). Quad protein migrated according to its expected MW as indicated by the arrow with no visible impurities (B) Direct binding ELISA using serially diluted Quad 56 protein with a fixed concentration of recombinant TNFa protein coated on plate. Quad 56 binds TNFa protein in a dose-dependent manner. (C) Direct binding ELISA comparing binding strength of three different bispecific tetravalent Quad formats (Quad 54: dAb×dAb, Quad 55: scFv×scFv and Quad 56: ScFv×dAb) to recombinant CD20. All three bispecific tetravalent Quad formats bind CD20 with similar binding strength and in a dose-dependent manner.

FIG. 29: SDS-PAGE analysis of monospecific tetravalent monomeric Ig scFv Quad 64 version 1 protein purified from culture supernatant (A). “Mononomeric Ig” refers to a multimer of a polypeptide of the invention that comprises an Fc, wherein CH2 comprises a hinge sequence but lacks a core hinge region; this advantageously prevents Fc regions from multimerizing together so that the multimerization is instead brought about by the SAM (eg, TD) domains of polypeptides in the multimer. Quad protein migrated according to its expected MW as indicated by the arrow with no visible impurities (B) Direct binding ELISA using serially diluted Quad 64 protein with a fixed concentration of recombinant CD20 protein coated on plate. Quad 64 binds CD20 protein in a dose-dependent manner.

FIG. 30: SDS-PAGE analysis of monospecific tetravalent monomeric Ig scFv Quad 65 version 2 protein purified from culture supernatant (A). Quad protein migrated according to its expected MW as indicated by the arrow with no visible impurities (B) Direct binding ELISA using serially diluted Quad 65 protein with a fixed concentration of recombinant CD20 protein coated on plate. Quad 65 binds CD20 protein in a dose-dependent manner.

FIG. 31: Schematic representation of Quad 68 and Quad 69 (A & B). The specificity of dAbs for PD-L1 and 4-1BB is indicated by arrows. (C & D) SDS-PAGE analysis of Quad 68 and Quad 69 protein purified from culture supernatant. Quad proteins migrated according to their expected MW as indicated by the arrows with no visible impurities.

FIG. 32: Alignment of p53 tetramerisation domain (TD) from different species. Sequence variations from human TD are highlighted in bold and underlined.

FIG. 33. Schematic representation of the molecular (A) and structural (B & C) arrangements of tetravalent and octavalent anti-TNF alpha dAb monomeric Ig Quads. Purified Quad proteins were analysed by SDS-PAGE (D). The tetravalent and octavalent Quad proteins migrated according to the expected molecular weight as indicated with no visible impurities. The core hinge region was removed in these formats and this is indicated in the figures with either a * or as CH2′. The Q92 chain contains a His-tag located at the C-terminus, which is not shown in the figure.

FIG. 34. SDS-PAGE analysis ofoctavalent bispecific anti-PDL1/4-1BB dAb monomeric Ig Quad (A) and octavalent monospecific anti-PDL1 dAb monomeric Ig Quad (B) proteins purified from culture supernatant. Quad proteins migrated according to their expected molecular weight as indicated by the arrow with no visible impurities.

FIG. 35. SDS-PAGE analysis of avelumab Fab monomeric Ig Quad (A) and Humira Fab monomeric Ig Quad (B) proteins purified from culture supernatant. Quad proteins migrated according to their expected molecular weight as indicated by the arrow with no visible impurities. Humira (adalimumab) Fab monomeric Ig Quad was further analysed by SEC where the fully assembled tetrameric protein eluted as a single peak at the expected molecular weight (315.8 kDa) with no visible detection of the dimeric or monomeric form.

FIG. 36. Schematic representation of the structural arrangements of aflibercept monomeric Ig Quad (Q96). The * denotes the hinge region is divoid of the core hinge region. Q96 Quad protein was analysed by SDS-PAGE where a single protein band at the expected molecular weight can be seen (B). Binding profile of Q96 to VEGF-A was analysed by ELISA binding assay where a dose-dependent binding can be seen.

FIG. 37. Schematic representation of the molecular arrangements of monovalent, tetravalent and octavalent anti-TNF alpha dAb Quads (A). All constructs contain a His-tag located at the C-terminus, which is not shown in the figure. Purified Quad proteins were analysed by SDS-PAGE where a single protein band at the expected molecular weight for monovalent, tetravalent and octavalent versions (Lanes 1-3 respectively) can be seen (B). TNF alpha binding assay using Q88 monovalent, tetravalent and octavalent Quad proteins (C) in addition to Q92 and Q92+Q93 (D) Quad proteins were performed by ELISA where a dose-dependent binding can be seen. The respective TNF alpha neutralization potency of the Quad proteins were analysed using WEHI cell-based bioassay (E & F, respectively). Enhancement in TNF alpha neutralization potency can be seen in Quads with increasing number of anti-TNF alpha dAb binding domains.

FIG. 38. Schematic representation of the molecular (A) and structural (B) arrangements of dodeca and hexadeca anti-TNF alpha dAb multimers (we alternatively call multimers, Quads). The light chain constant region is denoted by CL, which could comprise of either kappa constant region or lambda constant region. The Q142 construct contains a His-tag located at the C-terminus, which is not shown in the figure. Purified Quad proteins were analysed by SDS-PAGE (C). Dodeca valent anti-TNF alpha dAb Quads with either lambda (lane 1) or kappa (lane 2) constant region migrated on the SDS-PAGE gel according to the expected molecular weight. Hexadeca valent anti-TNF alpha dAb Quads with either lambda (lane 3) or kappa (lane 4) constant region also migrated on the SDS-PAGE gel according to the expected molecular weight. The TNF alpha neutralization potency of the dodeca and hexadeca anti-TNF alpha dAb Quad proteins with either kappa (D) or lambda (E) light chain constant region where analysed using WEHI cell-based bioassay. Potency enhancement with increasing anti-TNF alpha dAb binding domains can be seen.

FIG. 39. Schematic structural representation of dodeca-valent trispecific (A) and hexadeca-valent tetraspecific (B) multimers. For each format, the schematic structure of the monomeric building block is also shown. The regions within these molecules containing optional flexible linkers are indicated with arrows. The CL domain could be either a kappa or lambda C region. The dodeca-valent trispecific Quad contains three different dAbs labeled 1-3, which can bind either three different antigens on three different cells or bind three antigens on the same cell or three different epitopes on the same antigen. This format represents a 4+4+4 trispecific dodeca-valent Quad. The hexadeca-valent tetraspecific Quad contains four different dAbs labeled 1-4, which can bind either four different antigens on four different cells or bind four antigens on the same cell or four epitopes on the same antigen. This format represents a 4+4+4+4 tetraspecific hexadeca-valent Quad.

FIG. 40. Schematic structural representation of tetravalent non Ig-like Humira Fab-TD Quad (A). The schematic structure of the monomeric building block is also shown. The regions within this molecule containing optional flexible linkers are indicated with arrows (eg, each linker is a (G₄S linker as described herein). The * denotes absence of the core hinge region (ie, the presence of a lower hinge sequence and optionally also an upper hinge sequence). Purified Humira Fab-TD protein was analysed by SDS-PAGE where a single protein band at the expected molecular weight can be seen (B). TNFα binding assay using Humira Fab-TD and Humira Fab monovalent control proteins were performed by ELISA where a dose-dependent binding can be seen (C). The respective TNFα neutralization potency of the Quad proteins were analysed using WEHI cell-based bioassay (D). Enhancement in TNFα neutralization potency can be seen in Humira Fab-TD compared to Humira Fab monovalent control.

FIG. 41. Schematic structural representation of octavalent Fabs as non Ig-like Quad A) or as Ig-like Quad (B). For each format, the schematic structure of the monomeric building block is also shown. The regions within these molecules containing optional flexible linkers are indicated with arrows (eg, each linker is a (G₄S linker as described herein).

FIG. 42. Schematics of monomeric building block of formats A-AC as outlined in FIG. 22.

All polypeptide schematics and amino acid sequences herein are written N- to C-terminal. All nucleotide sequences herein are written 5′ to 3′.

### DETAILED DESCRIPTION

The invention relates to multimers such as tetramers of polypeptides and tetramers, octamers, dodecamers, hexadecamers or 20-mesr (eg, tetramers and octamers) of epitopes or effector domains (such as antigen binding sites (eg, antibody or TCR binding sites that specifically bind to antigen or pMHC, or variable domains thereof)) or peptides such as incretin, insulin or hormone peptides. In embodiments, multimers of the invention are usefully producible in eurkaryotic systems and can be secreted from eukaryotic cells in soluble form, which is useful for various industrial applications, such as producing pharmaceuticals, diagnostics, as imaging agents, detergents etc. Higher order multimers, such as tetramers or octamers of effector domains or peptides are useful for enhancing antigen or pMHC binding avidity. This may be useful for producing an efficacious medicine or for enhancing the sensitivity of a diagnostic reagent comprising the multimer, such as tetramer or octamer. An additional or alternative benefit is enhanced half-life in vivo when the multimers of the invention are administered to a human or animal subject, eg, for treating or preventing a disease or condition in the subject. Usefully, the invention can also provide for multi-specific (eg, bi- or tri-specific) multivalent binding proteins. Specificity may related to specificity of antigen or pMHC binding. By using a single engineered polypeptide comprising binding domains or peptides, the invention in certain examples usefully provides a means for producing multivalent (eg, bi-specific) proteins at high purity. Use of a single species of engineered polypeptide monomer avoids the problem of mixed products seen when 2 or more different polypeptide species are used to produce multi- (eg, bi-) specific or multivalent proteins.

The invention provides the following Clauses, Aspects, Paragraphs and Concepts (which are not intended to represent “Claims”; Claims are presented towards the end of this disclosure after the Examples and Tables). Any Clause herein can be combined with any Aspect or Concept herein. Any Aspect herein can be combined with any Concept herein.

Aspects:

1. A protein multimer of at least first, second, third and fourth copies of an effector domain (eg, a protein domain) or a peptide, wherein the multimer is multimerised by first, second, third and fourth self-associating tetramerisation domains (TDs) which are associated together, wherein each tetramerisation domain is comprised by a respective engineered polypeptide comprising one or more copies of said protein domain or peptide.

In an example, each TD is a TD of any one of proteins 1 to 119 listed in Table 2. In an example, each TD is a p53 TD or a homologue or orthologue thereof. In an example, each TD is a NHR2 TD or a homologue or orthologue thereof. In an example, each TD is a p63 TD or a homologue or orthologue thereof. In an example, each TD is a p73 TD or a homologue or orthologue thereof. In an example, each TD is not a NHR2 TD. In an example, each TD is not a p53 TD. In an example, each TD is not a p63 TD. In an example, each TD is not a p73 TD. In an example, each TD is not a p53, 63 or 73 TD. In an example, each TD is not a NHR2, p53, 63 or 73 TD.

By being “associated together”, the TDs in Aspect 1 multimerise first, second, third and fourth copies of the engineered polypeptide to provide a multimer protein, for example, a multimer that can be expressed intracellularly in a eukaryotic or mammalian cell (eg, a HEK293 cell) and/or which can be extracellularly secreted from a eukaryotic or mammalian cell (eg, a HEK293 cell) and/or which is soluble in an aqueous medium (eg, a eukaryotic or mammalian cell (eg, a HEK293 cell) culture medium). Examples are NHR TD, p53 TD, p63 TD and p73 TD (eg, human NHR TD, p53 TD, p63 TD and p73 TD) or an orthologue or homologue thereof.

In an example, the TD is not a p53 TD (or homologue or orthologue thereof), eg, it is not a human p53 TD (or homologue or orthologue thereof). In an example, the TD is a NHR2 TD or a homologue or orthologue thereof, but excluding a p53 TD or a homologue or orthologue thereof. In an example, the TD is a human NHR2 TD or a homologue or orthologue thereof, but excluding a human p53 TD or a homologue or orthologue thereof. In an example, the TD is human NHR2. In an example, the amino acid sequence of the TD is at least 80, 85, 90, 95, 96, 97, 98 or 99% identical to the sequence of human NHR2. In an example, the domain or peptide is not naturally comprised by a polypeptide that also comprise a NHR2 TD.

In an example, all of the domains of the polypeptide are human.

The engineered polypeptide may comprise one or more copies of said domain or peptide N-terminal to a copy of said TD. Additionally or alternatively, the engineered polypeptide may comprise one or more copies of said domain or peptide C-terminal to a copy of said TD. In an example, the engineered polypeptide comprises a first said domain or peptide and a TD, wherein the first domain or peptide is spaced by at least 10, 20, 30, 40, 50, 60, 70, 80, 90, 100, 200, 300, 400, 500, 600, 700, 800, 900 or 1000 contiguous amino acids from the TD, wherein there is no further said domain or peptide between the first domain or peptide and the TD.

In an example, the multimer (eg, tetramer of said engineered polypeptide) comprises 4 (but no more than 4) TDs (eg, identical TDs) and 4, 8, 12 or 16 (but no more than said 4, 8, 12 or 16 respectively) copies of said domain or peptide. In an example, each TD and each said domain or peptide is human.

In an example, the multimer, tetramer, octamer, dodecamer, hexadecamer or 20-mer (eg, tetramer or octamer) comprises first, second, third and fourth identical copies of an engineered polypeptide, the polypeptide comprising a TD and one (but no more than one), two (but no more than two), or more copies of the said protein domain or peptide. For example, a tetramer of the epitope or effector domain has 4 identical copies of the polypeptide comprising a TD and each polypeptide has 1 such epitope or effector domain. For example, an octamer of the epitope or effector domain has 4 identical copies of the polypeptide comprising a TD and each polypeptide has 2 such epitope or effector domain. For example, a dodecamer of the epitope or effector domain has 4 identical copies of the polypeptide comprising a TD and each polypeptide has 3 such epitope or effector domain. For example, a hexadecamer of the epitope or effector domain has 4 identical copies of the polypeptide comprising a TD and each polypeptide has 4 such epitope or effector domain. For example, a 20-mer of the epitope or effector domain has 4 identical copies of the polypeptide comprising a TD and each polypeptide has 5 such epitope or effector domain. Generally, for example, a X-mer of the epitope or effector domain has 4 identical copies of the polypeptide comprising a TD and each polypeptide has X/4 such epitope or effector domain, where X=any multiple of 4, eg, 4, 8, 12, 16, 20, 24, 28 or 32.

In some embodiments, by requiring just one type of engineered polypeptide to form the multimer, eg, tetramer or octamer, of the invention, the invention advantageously provides a format that can be readily isolated in pure (or highly pure, ie >90, 95, 96, 97, 98 or 99/6 purity) format, as well as a method for producing such a format in pure (or highly pure) form. Purity is indicated by the multimer of the invention not being in mixture in a composition with any other multimer or polypeptide monomer, or wherein the multimer of the invention comprises >90, 95, 96, 97, 98 or 99% of species in a composition comprising the multimer of the invention and other multimers and/or polypeptide monomers which comprise the engineered polypeptide. Thus, mixtures of different types of polypeptide in these embodiments are avoided or minimised. This advantageously also provides, therefore, plurality of multimers (eg, a plurality of tetramers or octamers or dodecamers or hexadecamers) that comprise only one (and no more than one) type of engineered polypeptide, wherein the multimers are monospecific (but multivalent) for antigen binding, or alternatively bi- or multi-specific for antigen binding. Thus, the invention provides a plurality of multimers (eg, a plurality of tetramers or octamers or dodecamers or hexadecamers, each polypeptide being at least tetra-valent for antigen binding and (i) bi-specific (ie, capable of specifically binding to 2 different antigens) or (ii) mono-specific and at least tetravalent for antigen binding. Herein, where antigen binding is mentioned this can instead be pMHC binding when the domain is a TCR V domain. Advantageously, the plurality is in pure form (ie, not mixed with multimers (eg, tetramers or octamers or dodecamers or hexadecamers) that comprise more than one type of polypeptide monomer. In an example, the multimer comprises at least 2 different types of antigen binding site. In an example, the multimer is bi-specific, tri-specific or tetra-specific. In an example, the multimer has an antigen binding site or pMHC binding site valency of 4, 6, 8, 10 or 12, preferably 4 or 8.

In an example, a peptide MHC (pMHC) is a class I or class II pMHC.

By the term “specifically binds,” as used herein, eg, with respect to a domain, antibody or binding site, is meant a domain, antibody or binding site which recognises a specific antigen (or pMHC) with a binding affinity of 1 mM or less as determined by SPR

Target binding ability, specificity and affinity (KD (also termed Kd), K_{off} and/or Kₒₙ) can be determined by any routine method in the art, eg, by surface plasmon resonance (SPR). The term “KD”, as used herein, is intended to refer to the equilibrium dissociation constant of a particular binding site/ligand, receptor/ligand or antibody/antigen interaction. In one embodiment, the surface plasmon resonance (SPR) is carried out at 25° C. In another embodiment, the SPR is carried out at 37° C. In one embodiment, the SPR is carried out at physiological pH, such as about pH7 or at pH7.6 (eg, using Hepes buffered saline at pH7.6 (also referred to as HBS-EP)). In one embodiment, the SPR is carried out at a physiological salt level, eg, 150 mM NaCl. In one embodiment, the SPR is carried out at a detergent level of no greater than 0.05% by volume, eg, in the presence of P20 (polysorbate 20; eg, Tween-20™) at 0.05% and EDTA at 3 mM. In one example, the SPR is carried out at 25° C. or 37° C. in a buffer at pH7.6, 150 mM NaCl, 0.05% detergent (eg, P20) and 3 mM EDTA. The buffer can contain 10 mM Hepes. In one example, the SPR is carried out at 25° C. or 37° C. in HBS-EP. HBS-EP is available from Teknova Inc (California; catalogue number H8022). In an example, the affinity (eg, of a VH/VL binding site) is determined using SPR by using any standard SPR apparatus, such as by Biacore™ or using the ProteOn XPR36™ (Bio-Rad®). The binding data can be fitted to 1:1 model inherent using standard techniques, eg, using a model inherent to the ProteOn XPR36™ analysis software.

In an example, a multimer, tetramer or octamer or dodecamer or hexadecamer or 20-mer of the invention is an isolated multimer, tetramer or octamer or dodecamer or hexadecamer or 20-mer. In an example, a multimer, tetramer or octamer of the invention consists of copies of said engineered polypeptide. Optionally the multimer, tetramer or octamer or dodecamer or hexadecamer or 20-mer of the invention comprises 4 or 8 or 12 or 16 or 20 but not more than 4 or 8 or 12 or 16 or 20 copies respectively of the engineered polypeptide.

By “engineered” is meant that the polypeptide is not naturally-occurring, for example the protein domain or peptide is not naturally comprised by a polypeptide that also comprises said TD.

Each said protein domain or peptide may be a biologically active domain or peptide (eg, biologically active in humans or animals), such as a domain that specifically binds to an antigen or peptide-MHC (pMHC), or wherein the domain is comprised by an antigen or pMHC binding site. In an alternative, the domain or peptide is a carbohydrate, glucose or sugar-regulating agent, such as an incretin or an insulin peptide. In an alternative, the domain or peptide is an inhibitor or an enzyme or an inhibitor of a biological function or pathway in humans or animals. In an alternative, the domain or peptide is an iron-regulating agent. Thus, in an example, each protein domain or peptide is selected from an antigen or pMHC binding domain or peptide; a hormone; a carbohydrate, glucose or sugar-regulating agent; an iron-regulating agent; and an enzyme inhibitor.

2. The multimer of any preceding Aspect, wherein the multimer is a tetramer, octamer, 12-mer, 16-mer or 20-mer (eg, a tetramer, octamer, 12-mer or 16-mer) of said domain or peptide.
3. The multimer of any Aspect 1 or 2, comprising a tetramer, octamer, 12-mer, 16-mer or 20-mer (eg, a tetramer, octamer, 12-mer or 16-mer) of an immunoglobulin superfamily binding site (eg, an antibody or TCR binding site, such as a scFv or scTCR).

The immunoglobulin superfamily (IgSF) is a large protein superfamily of cell surface and soluble proteins that are involved in the recognition, binding, or adhesion processes of cells. Molecules are categorized as members of this superfamily based on shared structural features with immunoglobulins (also known as antibodies); they all possess a domain known as an immunoglobulin domain or fold. Members of the IgSF include cell surface antigen receptors, co-receptors and co-stimulatory molecules of the immune system, molecules involved in antigen presentation to lymphocytes, cell adhesion molecules, certain cytokine receptors and intracellular muscle proteins. They are commonly associated with roles in the immune system.

T-cell receptor (TCR) domains can be Vα (eg. paired with a Vβ), Vβ (eg. paired with a Vα), Vγ (eg, paired with a Vδ) or Vδ (eg, paired with a Vγ).

4. The multimer of Aspect 3, wherein the binding site comprises a first variable domain paired with a second variable domain.

In a first example, the first and second variable domains are comprised by the engineered polypeptide. In another example, the first domain is comprised by the engineered polypeptide and the second domain is comprised a by a further polypeptide that is different from the engineered polypeptide (and optionally comprises a TD or is devoid of a TD).

In the alternative, the domains are constant region domains. In an alternative, the domains are FcAbs. In an alternative, the domains are non-Ig antigen binding sites or comprises by a non-Ig antigen binding site, eg, an affibody.

### Antigen Binding Sites & Effector Domains

In an example, the or each antigen binding site (or effector domain) is selected from the group consisting of an antibody variable domain (eg, a VL or a VH, an antibody single variable domain (domain antibody or dAb), a camelid VHH antibody single variable domain, a shark immunoglobulin single variable domain (NA V), a Nanobody™ or a camelised VH single variable domain); a T-cell receptor binding domain; an immunoglobulin superfamily domain; an agnathan variable lymphocyte receptor (J Immunol; 2010 Aug. 1; 185(3):1367-74; “Alternative adaptive immunity in jawless vertebrates; Herrin BR & Cooper M D.); a fibronectin domain (eg, an Adnectin™); an scFv; an (scFv)₂; an sc-diabody; an scFab; a centyrin and an antigen binding site derived from a scaffold selected from CTLA-4 (Evibody™); a lipocalin domain; Protein A such as Z-domain of Protein A (eg, an Affibody™ or SpA); an A-domain (eg, an Avimer™ or Maxibody™); a heat shock protein (such as and epitope binding domain derived from GroEI and GroES); a transferrin domain (eg, a trans-body); ankyrin repeat protein (eg, a DARPin™); peptide aptamer; C-type lectin domain (eg, Tetranectin™); human γ-crystallin or human ubiquitin (an affilin); a PDZ domain; scorpion toxin; and a kunitz type domain of a human protease inhibitor.

Further sources of antigen binding sites are variable domains and VH/VL pairs of antibodies disclosed in WO2007024715 at page 40, line 23 to page 43, line 23. This specific disclosure is incorporated herein by reference as though explicitly written herein to provide basis for epitope binding moieties for use in the present invention and for possible inclusion in claims herein.

A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain

The phrase “immunoglobulin single variable domain” or “antibody single variable domain” refers to an antibody variable domain (VH, VHH, VL) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004), nurse shark and Camelid VHH immunoglobulin single variable domains. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are still considered to be “domain antibodies” according to the invention. As used herein “VH includes camelid VHH domains. NA V are another type of immunoglobulin single variable domain which were identified in cartilaginous fish including the nurse shark. These domains are also known as Novel Antigen Receptor variable region (commonly abbreviated to V(NAR) or NARV). For further details see Mol. Immunol. 44, 656-665 (2006) and US20050043519A. CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001). Lipocalins are a family of extracellular proteins which transport small hydrophobic molecules such as steroids, bilins, retinoids and lipids. They have a rigid β-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. Sel. 17, 455-462 (2004) and EP1641818A1. Avimers™ are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins™) are derived from ankyrin which is a family of proteins that mediate attachment of integral membrane proteins to the cytoskeleton. A single ankyrin repeat is a 33 residue motif consisting of two a-helices and a β-turn. They can be engineered to bind different target antigens by randomising residues in the first a-helix and a β-turn of each repeat. Meir binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. Fibronectin is a scaffold which can be engineered to bind to antigen. Adnectins™ consist of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can be engineered to include upto 25 amino acids without affecting the overall fold of the microprotein. For further details of engineered knottin domains, see WO2008098796. Other epitope binding moieties and domains include proteins which have been used as a scaffold to engineer different target antigen binding properties include human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006).

In an example, the or each antigen binding site (or effector domain) comprises a non-Ig scaffolded, eg, is selected from the group consisting of Affibodies, Affilins, Anticalins, Atrimers, Avimers, Bicycle Peptides, Cys-knots, DARpins, Fibronectin type III, Fyomers, Kunitz Domain, OBodies, Aptamers, Adnectins, Armadillo Repeat Domain, Beta-Hairpin mimetics and Lipocalins.

5. The multimer of any preceding Aspect, wherein each polypeptide comprises first and second copies of said protein domain or peptide, wherein the polypeptide comprises in (N- to C-terminal direction) (i) a first of said copies—TD—the second of said copies; (ii) TD—and the first and second copies; or (iii) said first and second copies—TD.
6. The multimer of any preceding Aspect, wherein the TDs are NHR2 TDs and the domain or peptide is not a NHR2 domain or peptide; or wherein the TDs are p53 TDs and the domain or peptide is not a p53 domain or peptide.
7. The multimer of any preceding Aspect, wherein the engineered polypeptide comprises one or more copies of a second type of protein domain or peptide, wherein the second type of protein domain or peptide is different from the first protein domain or peptide.

For example, the polypeptide comprises in N-terminal direction (i) P1-TD-P2; or (ii) TD-P1-P2, wherein P1=a copy of a domain or peptide of the first type (ie, the type of domain or peptide of the multimer of Aspect 1); and P2=a copy of a domain or peptide of said second type.

8. The multimer of any preceding Aspect, wherein the domains are immunoglobulin superfamily domains.
9. The multimer of any preceding Aspect, wherein the domain or peptide is an antibody variable or constant domain (eg, an antibody single variable domain), a TCR variable or constant domain, an incretin, an insulin peptide, or a hormone peptide.
10. The multimer of any preceding Aspect, wherein the multimer comprises first, second, third and fourth identical copies of a said engineered polypeptide, the polypeptide comprising a TD and one (but no more than one), two (but no more than two) or more copies of the said protein domain or peptide.
11. The multimer of any preceding Aspect, wherein the engineered polypeptide comprises an antibody or TCR variable domain (V1) and a NHR2 TD.
12. The multimer of Aspect 11, wherein the polypeptide comprises (in N- to C-terminal direction) (i) V1-an optional linker-NHR2 TD; (ii) V1-an optional linker-NHR2 TD-optional linker-V2; or (iii) V1-an optional linker-V2—optional linker—NHR2 TD, wherein V1 and V2 are TCR variable domains and are the same or different, or wherein V1 and V2 are antibody variable domains and are the same or different.
13. The multimer of Aspect 12, wherein V1 and V2 are antibody single variable domains.
14. The multimer of aspect 11, wherein each engineered polypeptide comprises (in N- to C-terminal direction) V1-an optional linker-NHR2 TD, wherein V1 is an antibody or TCR variable domain and each engineered polypeptide is paired with a respective second engineered polypeptide that comprises V2, wherein V2 is a an antibody or TCR variable domain respectively that pairs with V1 to form an antigen or pMHC binding site, and optionally one polypeptide comprises an antibody Fc, or comprises antibody CH1 and the other polypeptide comprises an antibody CL that pairs with the CH1.
15. The multimer of any preceding Aspect, wherein the TD comprises (i) an amino acid sequence identical to SEQ ID NO: 10 or 126 or at least 80% identical thereto; or (ii) an amino acid sequence identical to SEQ ID NO: 120 or 123 or at least 80% identical thereto.
16. The multimer of any preceding Aspect, wherein the multimer comprises a tetramer, octamer, 12-mer, 16-mer or 20-mer (eg, a tetramer, octamer, 12-mer or 16-mer; or a tetramer or octamer) of an antigen binding site of an antibody selected from the group consisting of ReoPro™; Abciximab; Rituxan™; Rituximab; Zenapax™; Daclizumab; Simulect™; Basiliximab; Synagis™; Palivizumab; Remicade™; Infliximab; Herceptin™; Mylotarg™; Gemtuzumab; Campath™; Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara™; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; Kadcyla™; Ado-trastuzumab; Keytruda™, Opdivo™, Gazyva™ and Obinutuzumab. Optionally, the binding site of the polypeptide of the multimer comprises a VH of the binding site of the antibody and also the CH1 of the antibody (ie, in N- to C-terminal direction the VH-CH1 and SAM). In an embodiment, the polypeptide may be paired with a further polypeptide comprising (in N- to C-terminal direction a VL-CL, eg, wherein the CL is the CL of the antibody).

For example, a said protein domain of the engineered polypeptide is a V domain (a VH or VL) of an antibody binding site of an antibody selected from said group, wherein the multimer comprises a further V domain (a VL or VH respectively) that pairs with the V domain of the engineered polypeptide to form the antigen binding site of the selected antibody. Advantageously, therefore, the invention provides tetramer, octamer, 12-mer, 16-mer or 20-mer (eg, a tetramer, octamer, 12-mer or 16-mer; or tetramer or octamer) of a binding site of said selected antibody, which beneficially may have improved affinity, avidity and/or efficacy for binding its cognate antigen or for treating or preventing a disease or condition in a human or animal wherein the multimer is administered thereto to bind the cognate antigen in vivo.

For example, the multimer, tetramer, octamer, 12-mer, 16-mer or 20-mer (eg, a tetramer, octamer, 12-mer or 16-mer; or tetramer or octamer) comprises 4 (or said X/4 as described above) copies of an antigen binding site of an antibody, wherein the antibody is adalimumab, sarilumab, dupilumab, bevacizumab (eg, AVASTIN™), cetuximab (eg, ERBITUX™), tocilizumab (eg, ACTEMRA™) or trastuzumab (HERCEPTIN™). In an alternative the antibody is an anti-CD38 antibody, an anti-TNFa antibody, an anti-TNFR antibody, an anti-IL-4Ra antibody, an anti-IL-6R antibody, an anti-IL-6 antibody, an anti-VEGF antibody, an anti-EGFR antibody, an anti-PD-1 antibody, an anti-PD-L1 antibody, an anti-CTLA4 antibody, an anti-PCSK9 antibody, an anti-CD3 antibody, an anti-CD20 antibody, an anti-CD138 antibody, an anti-IL-1 antibody. In an alternative the antibody is selected from the antibodies disclosed in WO2007024715 at page 40, line 23 to page 43, line 23, the disclosure of which is incorporated herein by reference.

A binding site herein may, for example, be a ligand (eg, cytokine or growth factor, eg, VEGF or EGFR) binding site of a receptor (eg, KDR or Flt). A binding site herein may, for example, be a binding site of Eyelea™, Avastin™ or Lucentis™, eg, for ocular or oncological medical use in a human or animal. When the ligand or antigen is VEGF, the mutlimer, tetramer or octamer may be for treatment or prevention of a caner or ocular condition (eg, wet or dry AMD or diabetic retinopathy) or as an inhibitor of neovascularisation in a human or animal subject.

17. An isolated tetramer, octamer, dodecamer, hexadecamer or 20-mer of a TCR binding site, insulin peptide, incretin peptide or peptide hormone; or a plurality of said tetramers, octamers, dodecamers, hexadecamers or 20-mer s.

Several important peptide hormones are secreted from the pituitary gland. The anterior pituitary secretes three hormones: prolactin, which acts on the mammary gland; adrenocorticotropic hormone (ACTH), which acts on the adrenal cortex to regulate the secretion of glucocorticoids; and growth hormone, which acts on bone, muscle, and the liver. The posterior pituitary gland secretes antidiuretic hormone, also called vasopressin, and oxytocin. Peptide hormones are produced by many different organs and tissues, however, including the heart (atrial-natriuretic peptide (ANP) or atrial natriuretic factor (ANF)) and pancreas (glucagon, insulin and somatostatin), the gastrointestinal tract (cholecystokinin, gastrin), and adipose tissue stores (leptin). In an example, the peptide hormone of the invention is selected from prolactin, ACTH, growth hormone (somatotropin), vasopressin, oxytocin, glucagon, insulin, somatostatin, cholecystokinin, gastrin and leptin (eg, selected from human prolactin, ACTH, growth hormone, vasopressin, oxytocin, glucagon, insulin, somatostatin, cholecystokinin, gastrin and leptin).

In an example, the incretin is a GLP-1, GIP or exendin-4 peptide.

The invention provides, in embodiments, the following engineered multimers:—

An isolated tetramer, octamer, dodecamer, hexadecamer or 20-mer of an incretin.
An isolated tetramer, octamer, dodecamer, hexadecamer or 20-mer of an insulin peptide.
An isolated tetramer, octamer, dodecamer, hexadecamer or 20-mer of a GLP-1 (glucagon-like peptide-1 (GLP-1) peptide.
An isolated tetramer, octamer, dodecamer, hexadecamer or 20-mer of a GIP (glucose-dependent insulinotropic polypeptide) peptide.
An isolated tetramer, octamer, dodecamer, hexadecamer or 20-mer of an exendin (eg, exendin-4) peptide.
An isolated tetramer, octamer, dodecamer, hexadecamer or 20-mer of a peptide hormone.
An isolated tetramer, octamer, dodecamer, hexadecamer or 20-mer of a prolactin or prolactin peptide.
An isolated tetramer, octamer, dodecamer, hexadecamer or 20-mer of a ACTH or ACTH peptide.
An isolated tetramer, octamer, dodecamer, hexadecamer or 20-mer of a growth hormone or growth hormone peptide.
An isolated tetramer, octamer, dodecamer, hexadecamer or 20-mer of a vasopressin or vasopressin peptide.
An isolated tetramer, octamer, dodecamer, hexadecamer or 20-mer of an oxytocin or oxytocin peptide.
An isolated tetramer, octamer, dodecamer, hexadecamer or 20-mer of a glucagon or glucagon peptide.
An isolated tetramer, octamer, dodecamer, hexadecamer or 20-mer of a insulin or insulin peptide.
An isolated tetramer, octamer, dodecamer, hexadecamer or 20-mer of a somatostatin or somatostatin peptide.
An isolated tetramer, octamer, dodecamer, hexadecamer or 20-mer of a cholecystokinin or cholecystokinin peptide.
An isolated tetramer, octamer, dodecamer, hexadecamer or 20-mer of a gastrin or gastrin peptide.
An isolated tetramer, octamer, dodecamer, hexadecamer or 20-mer of a leptin or leptin peptide.
An isolated tetramer, octamer, dodecamer, hexadecamer or 20-mer of an antibody binding site (eg, a scFv or Fab).
An isolated tetramer, octamer, dodecamer, hexadecamer or 20-mer of a TCR binding site (eg, a scTCR).
An isolated tetramer, octamer, dodecamer, hexadecamer or 20-mer of a TCR Vα/Vβ binding site.
An isolated tetramer, octamer, dodecamer, hexadecamer or 20-mer of a TCR Vγ/Vδ binding site.
An isolated tetramer, octamer, dodecamer, hexadecamer or 20-mer of an antibody single variable domain binding site.
An isolated tetramer, octamer, dodecamer, hexadecamer or 20-mer of an FcAb binding site.

In an example of any of these tetramer, octamer, dodecamer, hexadecamer or 20-mer s, the domain or peptide is human. In an example of any of these tetramer, octamer, dodecamer, hexadecamer or 20-mer s, the tetramer, octamer, dodecamer, hexadecamer or 20-mer comprises a NHR2 TD (eg, a human NHR2). In an example of any of these tetramer, octamer, dodecamer, hexadecamer or 20-mer s, the tetramer, octamer, dodecamer, hexadecamer or 20-mer comprises a p53 TD (eg, a human p53 TD). In an example of any of these tetramer, octamer, dodecamer, hexadecamer or 20-mer s, the tetramer, octamer, dodecamer, hexadecamer or 20-mer comprises a p63 TD (eg, a human p63 TD). In an example of any of these tetramer, octamer, dodecamer, hexadecamer or 20-mers, the tetramer, octamer, dodecamer, hexadecamer or 20-mer comprises a p73 TD (eg, a human p73 TD). In an example of any of these tetramer, octamer, dodecamer, hexadecamer or 20-mer s, the tetramer, octamer, dodecamer, hexadecamer or 20-mer comprises a tetramer of TDs (eg, human NHR2 TDs), whereby the domains or peptides form a multimer of 4 or 8 domains or peptides.

In an example, the plurality is pure, eg, is not in mixture with multimers of said binding site or peptide wherein the multimers comprise more than one type of polypeptide monomer.

18. The multimer, tetramer, octamer, dodecamer, hexadecamer or 20-mer of any preceding Aspect, wherein the multimer, tetramer, octamer, dodecamer, hexadecamer or 20-mer is
(a) soluble in aqueous solution (eg, an aqueous eukaryotic cell growth medium or buffer);
(b) secretable from a eukaryotic cell; and/or
(c) an expression product of a eukaryotic cell.

In an example the multimer, tetramer, octamer, dodecamer, hexadecamer or 20-mer is secretable from a HEK293T (or other eukaryotic, mammalian, CHO or Cos) cell in stable form as indicated by a single band at the molecular weight expected for said multimer, tetramer, octamer, dodecamer, hexadecamer or 20-mer on a PAGE gel using a sample of supernatant from such cells and detected using Western Blot.

19. A tetramer, octamer, dodecamer, hexadecamer or 20-mer (eg, a tetramer or octamer) of
(a) TCR V domains or TCR binding sites, wherein the tetramer, octamer, dodecamer, hexadecamer or 20-mer is soluble in aqueous solution (eg, an aqueous eukaryotic cell growth medium or buffer);
(b) antibody single variable domains, wherein the tetramer, octamer, dodecamer, hexadecamer or 20-mer is soluble in aqueous solution (eg, an aqueous eukaryotic cell growth medium or buffer);
(c) TCR V domains or TCR binding sites, wherein the tetramer, octamer, dodecamer, hexadecamer or 20-mer is capable of being intracellularly and/or extracellularly expressed by HEK293 cells; or
(d) antibody variable domains (eg, antibody single variable domains), wherein the tetramer, octamer, dodecamer, hexadecamer or 20-mer is capable of being intracellularly and/or extracellularly expressed by HEK293 cells.

An example of the medium is SFMII growth medium supplemented with L-glutamine (eg, complete SFMII growth medium supplemented with 4 mM L-glutamine). In an example, the medium is serum-free HEK293 cell culture medium. In an example, the medium is serum-free CHO cell culture medium.

For example, a cell herein is a human cell, eg, a HEK293 cell (such as a HEK293T cell).

20. The multimer, tetramer, octamer, dodecamer, hexadecamer or 20-mer of any preceding Aspect, wherein the tetramer, octamer, dodecamer, hexadecamer or 20-mer is bi-specific for antigen or pMHC binding.
21. The multimer, tetramer, octamer, dodecamer, hexadecamer or 20-mer of any preceding Aspect, wherein the domains are identical.
22. The multimer, tetramer, octamer, dodecamer, hexadecamer or 20-mer of any preceding Aspect, wherein the multimer, tetramer, octamer, dodecamer, hexadecamer or 20-mer comprises eukaryotic cell glycosylation.

For example, the glycosylation is CHO cell glycosylation. For example, the glycosylation is HEK (eg, HEK293, such as HEK293T) cell glycosylation. For example, the glycosylation is Cos cell glycosylation. For example, the glycosylation is Picchia cell glycosylation. For example, the glycosylation is Sacchaaromyces cell glycosylation.

23. The multimer, tetramer, octamer, dodecamer, hexadecamer or 20-mer of Aspect 22, wherein the cell is a HEK293 cell.
24. A plurality of multimers, tetramer, octamer, dodecamer, hexadecamer or 20-mer of any preceding Aspect.
25. A pharmaceutical composition comprising the multimer(s), tetramer(s), octamer(s), dodecamer(s), hexadecamer(s) or 20-mer (s) of any preceding Aspect and a pharmaceutically acceptable carrier, diluent or excipient.
26. A cosmetic, foodstuff, beverage, cleaning product, detergent comprising the multimer(s), tetramer(s), octamer(s), dodecamer(s), hexadecamer(s) or 20-mer (s) of any one of Aspects 1 to 24.
27. A said engineered (and optionally isolated) polypeptide or a monomer (optionally isolated) of a multimer, tetramer, octamer, dodecamer, hexadecamer or 20-mer of any preceding Aspect.

The monomer is an engineered polypeptide as disclosed herein, comprising a said protein domain or peptide and further comprising a TD.

Optionally, the engineered polypeptide comprises (in N- to C-terminal direction) a variable domain (V1)—a constant domain (C) (eg, a CH1 or Fc)—optional linker—TD.

28. An engineered (and optionally isolated) engineered polypeptide (P1) which comprises (in N- to C-terminal direction):—
(a) TCR V1-TCR C1—antibody C (eg, CH, CH1 (such as IgG CH1) or CL (such as Cλ or a Cκ))—optional linker—TD, wherein

### (i) V1 is a Vα and C1 is a Cα;

### (ii) V1 is a Vβ and C1 is a Cβ;

(iii) V1 is a Vγ and C1 is a Cγ; or

### (iv) V1 is a Vδ and C1 is a Cδ;

or
(b) TCR V1—antibody C (eg, CH, CH1 (such as IgG CH1) or CL (such as Cλ or a Cκ))—optional linker—TD, wherein

### (i) V1 is a Vα;

### (ii) V1 is a Vβ;

(iii) V1 is a Vγ; or

### (iv) V1 is a Vδ;

or
(c) antibody V1—antibody C (eg, CH, CH1 (such as IgG CH1) or CL (such as Cλ or a Cκ))—optional linker—TD, wherein

### (i) V1 is a VH; or

### (ii) V1 is a VL (eg, a Vλ or a Vκ);

or
(d) antibody V1—optional antibody C (eg, CH, CH1 (such as IgG CH1) or CL (such as Cλ or a Cκ))—antibody Fc (eg, an IgG Fc)—optional linker—TD, wherein

### (i) V1 is a VH; or

### (ii) V1 is a VL (eg, a Vλ or a Vκ);

or
(e) antibody V1—antibody CL (eg, a Cλ or a Cκ)—optional linker—TD, wherein

### (i) V1 is a VH; or

### (ii) V1 is a VL (eg, a Vλ or a Vκ);

or
(f) TCR V1-TCR C1—optional linker—TD, wherein

### (i) V1 is a Vα and C1 is a Cα;

### (ii) V1 is a Vβ and C1 is a Cβ;

(iii) V1 is a Vγ and C1 is a Cγ; or

### (iv) V1 is a Vδ and C1 is a Cδ.

In (a) or (b), in an example, the TCR V is comprised by an single chain TCR binding site (scTCR) that specifically binds to a pMHC, wherein the binding site comprises TCR V-linker—TCRV. In an example, the engineered polypeptide comprises (in N- to C-terminal direction) (i) V1—linker—V—optional C—optional linker—TD, or (ii) Va-linker-V1—optional C-optional linker—TD, wherein Va is a TCR V domain and C is an antibody C domain (eg, a CH1 or CL) or a TCR C.

Preferably, the antibody C is CH1 (eg, IgG CH1).

In an example the multimer, tetramer, octamer, dodecamer, hexadecamer or 20-mer has a size of no more than 155 kDa, eg, wherein said protein domain is an antibody variable domain comprising a CDR3 of at least 16, 17, 18, 19, 20, 21 or 22 amino acids, such as a Camelid CDR3 or bovine CDR3.

In an example, the multimer, tetramer, octamer, dodecamer, hexadecamer or 20-mer comprises TCR binding sites and antibody binding sites. For example, each polypeptide comprises a TCR V (eg, comprised by a scTCR that specifically binds a pMHC) and an antibody V (eg, comprised by a scFv or paired with a second V domain comprised by a said second polypeptide to form a V/V paired binding site that specifically binds to an antigen). In an example, the pMHC comprises a RAS peptide. In an example the antigen is selected from the group consisting of PD-1, PD-L1 or any other antigen disclosed herein. For example, the antigen is PD-1 and the pMHC comprises a RAS peptide.

29. The polypeptide of Aspect 28, wherein the engineered polypeptide P1 is paired with a further polypeptide (P2), wherein P2 comprises (in N- to C-terminal direction):—
(g) TCR V2-TCR C2—antibody CL (eg, a Cλ or a Cκ), wherein P1 is according to (a) recited in Aspect 28 and
(i) V2 is a Vα and C2 is a Cα when P1 is according to (a)(ii);
(ii) V2 is a Vβ and C2 is a Cβ when P1 is according to (a)(i);
(iii) V2 is a Vγ and C2 is a Cγ when P1 is according to (a)(iv); or
(iv) V2 is a Vδ and C2 is a Cδ when P1 is according to (a)(iii);
or
(h) TCR V2—antibody CL (eg, a Cλ or a Cκ), wherein P1 is according to (b) recited in Aspect 28
and
(i) V2 is a Vα when P1 is according to (b)(ii);
(ii) V2 is a Vβ when P1 is according to (b)(i);
(iii) V2 is a Vγ when P1 is according to (b)(iv); or
(iv) V2 is a Vδ when P1 is according to (b)(iiii);
or
(i) Antibody V2-CL (eg, a Cλ or a Cκ), wherein P1 is according to (c) recited in Aspect 28 and
(i) V2 is a VH when P1 is according to (c)(ii); or
(ii) V2 is a VL (eg, a Vλ or a Vκ) when P1 is according to (c)(i);
or
(j) Antibody V2—optional CL (eg, a Cλ or a Cκ), wherein P1 is according to (d) recited in Aspect 28 and
(i) V2 is a VH when P1 is according to (d)(ii); or
(ii) V2 is a VL (eg, a Vλ or a Vκ) when P1 is according to (d)(i); or
(k) Antibody V2-CH1 (eg, IgG CH1), wherein P1 is according to (e) recited in Aspect 28 and
(i) V2 is a VH when P1 is according to (e)(ii); or
(ii) V2 is a VL (eg, a Vλ or a Vκ) when P1 is according to (e)(i);
or
(1) TCR V2-TCR C2, wherein P1 is according to (f) recited in Aspect 28 and
(i) V2 is a Vα and C2 is a Cα when P1 is according to (f)(ii);
(ii) V2 is a Vβ and C2 is a Cβ when P1 is according to (f)(i);
(iii) V2 is a Vγ and C2 is a Cγ when P1 is according to (f)(iii); or
(iv) V2 is a Vδ and C2 is a Cδ when P1 is according to (f)(iv).

Optionally, V1 and V2 form a paired variable domain binding site that is capable of specifically binding to an antigen or pMHC. In an example, V1 and V2 are variable domains of an antibody, eg, selected from the group consisting of ReoPro™; Abciximab; Rituxan™; Rituximab; Zenapax™; Daclizumab; Simulect™; Basiliximab; Synagis™; Palivizumab; Remicade™; Infliximab; Herceptin™; Mylotarg™; Gemtuzumab; Campath™; Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara™; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; Kadcyla™; Ado-trastuzumab; Keytruda™, Opdivo™, Gazyva™ and Obinutuzumab. Optionally, the binding site of the polypeptide of the multimer comprises a VH of the binding site of the antibody and also the CH1 of the antibody (ie, in N- to C-terminal direction the VH-CH1 and SAM). In an embodiment, the polypeptide may be paired with a further polypeptide comprising (in N- to C-terminal direction a VL-CL, eg, wherein the CL is the CL of the antibody).

In one embodiment, the antibody is Avastin.

In one embodiment, the antibody is Actemra.

In one embodiment, the antibody is Erbitux.

In one embodiment, the antibody is Lucentis.

In one embodiment, the antibody is sarilumab.

In one embodiment, the antibody is dupilumab.

In one embodiment, the antibody is alirocumab.

In one embodiment, the antibody is bococizumab.

In one embodiment, the antibody is evolocumab.

In one embodiment, the antibody is pembrolizumab.

In one embodiment, the antibody is nivolumab.

In one embodiment, the antibody is ipilimumab.

In one embodiment, the antibody is remicade.

In one embodiment, the antibody is golimumab.

In one embodiment, the antibody is ofatumumab.

In one embodiment, the antibody is Benlysta.

In one embodiment, the antibody is Campath.

In one embodiment, the antibody is rituximab.

In one embodiment, the antibody is Herceptin.

In one embodiment, the antibody is durvalumab.

In one embodiment, the antibody is daratumumab.

In an example, any binding domain herein (eg, a dAb or scFv or Fab) or V1 is capable (itself when a single variable domain, or when paired with V2) of specifically binding to an antigen selected from the group consisting of ABCF1; ACVR1; ACVR1B; ACVR2; ACVR2B; ACVRL1; ADORA2A; Aggrecan; AGR2; AICDA; AWI; AIG1; AKAP1; AKAP2; AIYIH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BM P1; BMP2; BMP3B (GDFIO); BMP4; BMP6; BM P8; BMPRIA; BMPRIB; BM PR2; BPAG1 (plectin); BRCA1; CI9orflO (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-id); CCL16 (HCC-4); CCL17 (TARC); CCL18 (PARC); CCL 19 (M IP-3b); CCL2 (MCP-1); MCAF; CCL20 (MIP-3a); CCL21 (MIP-2); SLC; exodus-2; CCL22 (MDC/STC-1); CCL23 (M PIF-1); CCL24 (MPIF-2 I eotaxin-2); CCL25 (TECK); CCL26 (eotaxin-3); CCL27 (CTACK/ILC); CCL28; CCL3 (MIP-la); CCL4 (M IP-lb); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CM KBR5/ChemR13); CCR6 (CMKBR6/CKR-L3/STRL22/DRY6); CCR7 (CKR7/EBI1); CCR8 (CM KBR8/TERI/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p2IWap1/Cip1); CDKN1B (p27Kip1); CDKNIC; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYDi); CX3CR1 (V28); CXCL1 (GRO1); CXCLIO (IP-10); CXCL11 (1-TAC/IP-9); CXCL12 (SDF1); CXCL13; CXCL14; CXCL16; CXCL2 (GR02); CXCL3 (GR03); CXCL5 (ENA-78 I LIX); CXCL6 (GCP-2); CXCL9 (MIG); CXCR3 (GPR9/CKR-L2); CXCR4; CXCR6 (TYMSTR ISTRL33 I Bonzo); CYB5; CYC1; CYSLTR1; DAB2IP; DES; DKFZp451J0118; DNCL1; DPP4; E2F1; ECGF1; EDG1; EFNAI; EFNA3; EFNB2; EGF; EGFR; ELAC2; ENG; EN01; EN02; EN03; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FU12584; FU25530; FLRT1 (fibronectin); FLT1; FOS; FOSL1 (FRA-I); FY (DARC); GABRP (GABAa); GAGEB1; GAGEC1; GALNAC4S-65T; GATA3; GDF5; GFI1; GGT1; GM-CSF; GNAS1; GNRH1; GPR2 (CCRIO); GPR31; GPR44; GPR81 (FKSG80); GRCCIO (CIO); GRP; GSN (Gelsolin); GSTP1; HAVCR2; HDAC4; EDAC5; HDAC7A; HDAC9; HGF; HIF1A; HIP1; histamine and histamine receptors; HLA-A; HLA-DRA; HM74; HMOX1; HUMCYT2A; ICEBERG; ICOSL; 1D2; IFN-a; IFNA1; IFNA2; IFNA4; IFNA5; IFNA6; IFNA7; IFNB1; IFNgamma; TFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; ILIA; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2 IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; TL7R; IL8; IL8RA; IL8RB; IL8RB; IL9; IL9R; ILK; INHA; INHBA; INSL3; INSL4; IRAKI; IRAK2; ITGA1; ITGA2; 1TGA3; ITGA6 (a6 integrin); ITGAV; ITGB3; ITGB4 (b 4 integrin); JAG1; JAK1; JAK3; JUN; K6HF; KAI1; KDR; MTLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; M IB1; midkine; M IF; M IP-2; MK167 (Ki-67); MMP2; M MP9; MS4A1; MSMB; MT3 (metallothionectin-ifi); MTSS 1; M UC 1 (mucin); MYC; MYD88; NCK2; neurocan; NFKB 1; NFKB2; NGFB (NGF); NGFR; NgR-Lingo; NgR-Nogo66 (Nogo); NgR-p75; NgR-Troy; NM E1 (NM23A); NOX5; NPPB; NROB1; NROB2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PARTI; PATE; PAWR; PCA3; PCNA; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p2IRac2); RARB; RGS1; RGS13; RGS3; RNF110 (ZNF144); ROB02; S100A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINIA3; SERPINB5 (maspin); SERPINE1 (PAT-i); SERPINF1; SHBG; SLA2; SLC2A2; SLC33A1; SLC43A1; SLIT2; SPP1; SPRRIB (Spri); ST6GAL1; STAB1; STAT6; STEAP; STEAP2; TB4R2; TBX21; TCPIO; TDGF1; TEK; TGFA; TGFB1; TGFB1I1; TGFB2; TGFB3; TGFBI; TGFBR1; TGFBR2; TGFBR3; TH1L; THBS1 (thrombospondin-1); THBS2; THBS4; THPO; TIE (Tie-i); T]MP3; tissue factor; TLRIO; TLR2; TLR3; TLR4; TLR5; TLR6; TLR7; TLR8; TLR9; TNF; TNF-a (also referred to herein as TNF alpha or TNFα); TNFAIP2 (B94); TNFAIP3; TNFRSF1 1A; TNFRSF1A; TNFRSF1B; TNFRSF21; TNFRSF5; TNFRSF6 (Fas); TNFRSF7; TNFRSF8; TNFRSF9; TNFSFIO (TRAIL); TNFSF1 1 (TRANCE); TNFSF12 (AP03L); TNFSF13 (April); TNFSF13B; TNFSF14 (HVEM-L); TNFSF1 5 (VEGI); TNFSF1 8; TNFSF4 (0X40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase lia); TP53; TPM 1; TPM2; TRADD; TRAF1; TRAF2; TRAF3; TRAF4; TRAF5; TRAF6; TREM 1; TREM2; TRPC6; TSLP; TWEAK; VEGF; VEGFB; VEGFC; versican; VHL C5; VLA-4; XCL1 (lymphotactin); XCL2 (SCM-lb); XCR1 (GPR5/CCXCR1); YY1; and ZFPM2.

For example in any configuration of the invention, the multimer, octamer, dodecamer, hexadecamer or 20-mer specifically binds to first and second (eg, for an octamer, dodecamer, hexadecamer or 20-mer); optionally, first, second and third (eg, for a dodecamer, hexadecamer or 20-mer); or optionally, first, second, third and fourth (eg, for a hexadecamer or 20-mer); or optionally, first, second, third, fourth and fifth (eg, for a 20-mer) epitopes or antigens, each of which is selected from the group consisting of EpCAM and CD3; CD19 and CD3; VEGF and VEGFR2; VEGF and EGFR; CD138 and CD20; CD138 and CD40; CD20 and CD3; CD38 and CD138; CD38 and CD20; CD38 and CD40; CD40 and CD20; CD19 and CD20; CD-8 and IL-6; PDL-1 and CTLA-4; CTLA-4 and BTN02; CSPGs and RGM A; IGF1 and IGF2; IGF1 and/or 2 and Erb2B; IL-12 and IL-18; IL-12 and TWEAK; IL-13 and ADAM8; IL-13 and CL25; IL-13 and IL-1beta; IL-13 and IL-25; IL-13 and IL-4; IL-13 and IL-5; IL-13 and IL-9; IL-13 and LHR agonist; IL-13 and MDC; IL-13 and MIF; IL-13 and PED2; IL-13 and SPRR2a; IL-13 and SPRR2b; IL-13 and TARC; IL-13 and TGF-beta; IL-1 alpha and IL-1 beta; MAG and RGM A; NgR and RGM A; NogoA and RGM A; OMGp and RGM A; RGM A and RGM B; Te38 and TNF alpha; TNF alpha and IL-12; TNF alpha and IL-12p40; TNF alpha and IL-13; TNF alpha and IL-15; TNF alpha and IL-17; TNF alpha and IL-18; TNF alpha and IL-1 beta; TNF alpha and IL-23; TNF alpha and M IF; TNF alpha and PEG2; TNF alpha and PGE4; TNF alpha and VEGF; and VEGFR and EGFR; TNF alpha and RANK ligand; TNF alpha and Blys; TNF alpha and GP130; TNF alpha and CD-22; and TNF alpha and CTLA-4

For example, the first epitope or antigen is selected from the group consisting of CD3; CD16; CD32; CD64; and CD89; and the second epitope or antigen is selected from the group consisting of EGFR; VEGF; IGF-1R; Her2; c-Met (aka HGF); HER3; CEA; CD33; CD79a; CD19; PSA; EpCAM; CD66; CD30; HAS; PSMA; GD2; ANG2; IL-4; IL-13; VEGFR2; and VEGFR3.

In an example, any binding domain herein (eg, a dAb or scFv or Fab) or V1 is capable (itself when a single variable domain, or when paired with V2) of specifically binding to an antigen selected from the group consisting of human IL-1A, IL-1β, IL-1RN, IL-6, BLys, APRIL, activin A, TNF alpha, a BMP, BMP2, BMP7, BMP9, BMP10, GDF8, GDF11, RANKL, TRAIL, VEGFA, VEGFB or PGF; optionally the multimer comprises a cytokine amino acid sequence (eg, C-terminal to a TD), such as IL-2 or an IL2-peptide; and the multimer, octamer, dodecamer, hexadecamer or 20-mer is for treating or preventing a cancer in a human subject. In an example the said effector or protein domain is capable of binding to such an antigen; optionally the multimer comprises a cytokine amino acid sequence (eg, C-terminal to a TD), such as IL-2 or an IL2-peptide; and the multimer, tetramer, octamer, dodecamer, hexadecamer or 20-mer is for treating or preventing a cancer in a human subject.

30. A multimer (eg, a dimer, trimer, tetramer, octamer, dodecamer, hexadecamer or 20-mer) of P1 as defined in Aspect 28; or of P1 paired with P2 as defined in Aspect 29; or a plurality of said multimers, optionally wherein the multimer is according to any one of aspects 1 to 24.

Preferably the multimer is a tetramer of the engineered polypeptide and/or effector domain. In one example, the plurality of tetramers are not in mixture with monomers, dimers or trimers of the polypeptide,

In one example the multimer, eg, tetramer, is a capable of specifically binding to two different pMHC.

31. A nucleic acid encoding an engineered polypeptide or monomer of any one of Aspects 27 to 29, optionally wherein the nucleic acid is comprised by an expression vector for expressing the polypeptide.

In an example, the nucleic acid is a DNA, optionally operably connected to or comprising a promoter for expression of the polypeptide or monomer. In another example the nucleic acid is a RNA (eg, mRNA).

32. A eukaryotic host cell comprising the nucleic acid or vector of Aspect 31 for intracellular and/or secreted expression of the multimer, tetramer, octamer, dodecamer, hexadecamer or 20-mer, engineered polypeptide or monomer of any one of Aspects 1 to 24.
33. Use of a nucleic acid or vector according to aspect 31 in a method of manufacture of protein multimers for producing intracellularly expressed and/or secreted multimers, wherein the method comprises expressing the multimers in and/or secreting the multimers from eukaryotic cells comprising the nucleic acid or vector.
34. Use of a nucleic acid or vector according to aspect 31 in a method of manufacture of protein multimers for producing glycosylated multimers in eukaryotic cells comprising the nucleic acid or vector.

Mammalian glycosylation of the invention is useful for producing medicines comprising or consisting of the multimers, tetramer, octamer, dodecamer, hexadecamer or 20-mer of the invention for medical treatment or prevention of a disease or condition in a mammal, eg, a human. The invention thus provides such a method of use as well as the multimer, tetramer, octamer, dodecamer, hexadecamer or 20-mer of the invention for this purpose. Similarly, intracellular and/or secreted expression in one or more host cells (or cell lines thereof) that are mammalian according to the invention is useful for producing such medicines. Particularly useful is such expression in HEK293, CHO or Cos cells as these are commonly used for production of medicaments.

In an embodiment, the invention comprises a detergent or personal healthcare product comprising a multimer, tetramer, octamer, dodecamer, hexadecamer or 20-mer of the invention. In an embodiment, the invention comprises a foodstuff or beverage comprising a multimer, tetramer, octamer, dodecamer, hexadecamer or 20-mer of the invention.

In an example, the multimer, monomer, dimer, trimer, tetramer, octamer, dodecamer, hexadecamer or 20-mer, polypeptide, composition, mixture, use or method of the present invention is for an industrial or domestic use, or is used in a method for such use. For example, it is for or used in agriculture, oil or petroleum industry, food or drink industry, clothing industry, packaging industry, electronics industry, computer industry, environmental industry, chemical industry, aerospace industry, automotive industry, biotechnology industry, medical industry, healthcare industry, dentistry industry, energy industry, consumer products industry, pharmaceutical industry, mining industry, cleaning industry, forestry industry, fishing industry, leisure industry, recycling industry, cosmetics industry, plastics industry, pulp or paper industry, textile industry, clothing industry, leather or suede or animal hide industry, tobacco industry or steel industry.

35. A mixture comprising (i) a eukaryotic cell line encoding an engineered polypeptide according to any one of Aspects 27 to 29; and (ii) multimers, tetramers, octamers, dodecamers, hexadecamers or 20-mer s as defined in any one of Aspects 1 to 24.
36. The mixture of Aspect 35, wherein the cell line is in a medium comprising secretion products of the cells, wherein the secretion products comprise said multimers, tetramers, octamers, dodecamers, hexadecamers or 20-mer s.
37. The multimer, tetramer, octamer, dodecamer, hexadecamer or 20-mer of any one of aspects 1 to 24 for medical use.
38. A method producing
(a) TCR V domain multimers, the method comprising the soluble and/or intracellular expression of TCR V-NHR2 TD or TCR V-p53 TD fusion proteins expressed in eukaryotic cells, the method optionally comprising isolating a plurality of said multimers;
(b) antibody V domain multimers, the method comprising the soluble and/or intracellular expression of antibody V (eg, a single variable domain)-NHR2 TD or V-p53 TD fusion proteins expressed in eukaryotic cells, the method optionally comprising isolating a plurality of said multimers;
(c) incretin peptide (eg, GLP-1, GIP or insulin) multimers, the method comprising the soluble and/or intracellular expression of incretin peptide-NHR2 TD or incretin peptide-p53 TD fusion proteins expressed in eukaryotic cells, such as HEK293T cells; the method optionally comprising isolating a plurality of said multimers; or
(d) peptide hormone multimers, the method comprising the soluble and/or intracellular expression of peptide hormone-NHR2 TD or peptide hormone-p53 TD fusion proteins expressed in eukaryotic cells, such as HEK293T cells; the method optionally comprising isolating a plurality of said multimers.
39. Use of self-associating tetramerisation domains (TD) (eg, NHR2 TD, p53 TD, p63 TD or p73 TD or a homologue or orthologue thereof) in a method of the manufacture of a tetramer of polypeptides, for producing a higher yield of tetramers versus monomer and/or dimer polypeptides.
40. Use of an engineered polypeptide in a method of the manufacture of a tetramer of a polypeptide comprising multiple copies of a protein domain or peptide, for producing a higher yield of tetramers versus monomer and/or dimer polypeptides, wherein the engineered polypeptide comprises one or more copies of said protein domain or peptide and further comprises a self-associating tetramerisation domains (TD) (eg, NHR2 TD, p53 TD, p63 TD or p73 TD or a homologue or orthologue).
41. Use of self-associating tetramerisation domains (TD) (eg, NHR2 TD, p53 TD, p63 TD or p73 TD or a homologue or orthologue thereof) in a method of the manufacture of a tetramer of a polypeptide, for producing a plurality of tetramers that are not in mixture with monomers, dimers or trimers.
42. Use of an engineered polypeptide in a method of the manufacture of a tetramer of a polypeptide comprising multiple copies of a protein domain or peptide, for producing a plurality of tetramers that are not in mixture with monomers, dimers or trimers, wherein the engineered polypeptide comprises one or more copies of said protein domain or peptide and further comprises a self-associating tetramerisation domains (TD) (eg, NHR2 TD, p53 TD, p63 TD or p73 TD or a homologue or orthologue).
43. The use of any one of Aspects 39 to 42, wherein the yield of tetramers is at least 10, 20, 30, 40 or 50× the yield of monomers and/or dimers.
44. The use of any one of Aspects 39 to 43, wherein the ratio of tetramers produced:monomers and/or dimers produced in the method is at least 90:10 (eg, at least 95:5 or 98:2, or 99:1).
45. The use of any one of Aspects 39 to 44, wherein each monomer has a size of no more than 40, 35, 30, 25 or 20 kDa.
46. The use of any one of Aspects 39 to 45, wherein each tetramer has a size of no more than 200, 160, 155 or 150 kDa.
47. The use of any one of Aspects 39 to 46, wherein the method comprises expressing the tetramers from a eukaryotic cell line.
48. A multivalent heterodimeric soluble T cell receptor capable of binding pMHC complex comprising:
(i) TCR extracellular domains;
(ii) immunoglobulin constant domains; and
(iii) an NHR2 multimerisation domain of ETO.
49. A multimeric immunoglobulin, comprising
(i) immunoglobulin variable domains; and
(ii) an NHR2 multimerisation domain of ETO.
50. A method for assembling a soluble, multimeric polypeptide, comprising:
(a) providing a monomer of the said multimeric polypeptide, fused to an NHR2 domain of ETO;
(b) causing multiple copies of said monomer to associate, thereby obtaining a multimeric, soluble polypeptide.

The invention further provides

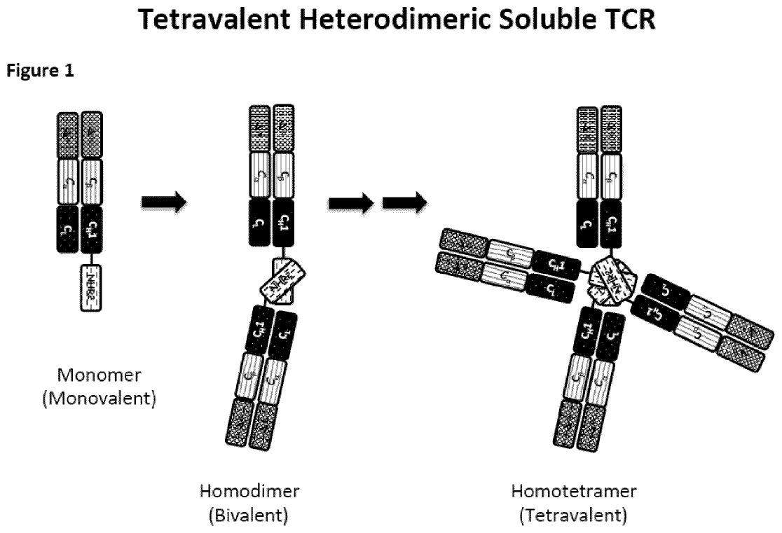

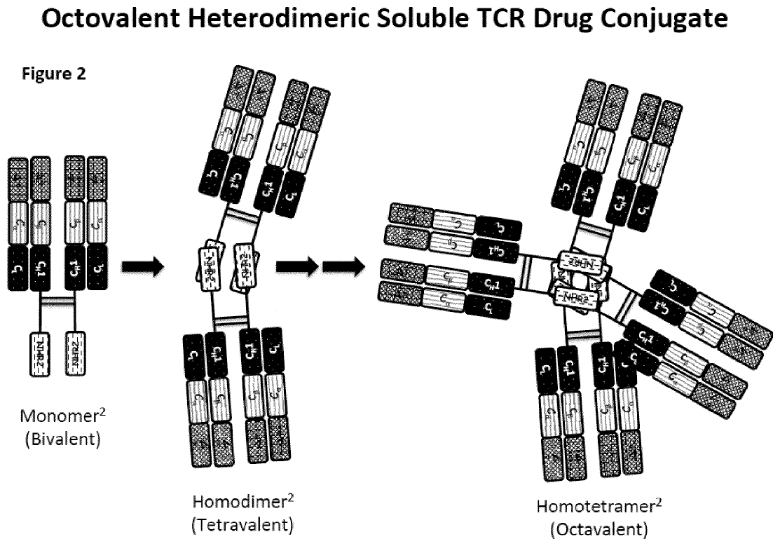

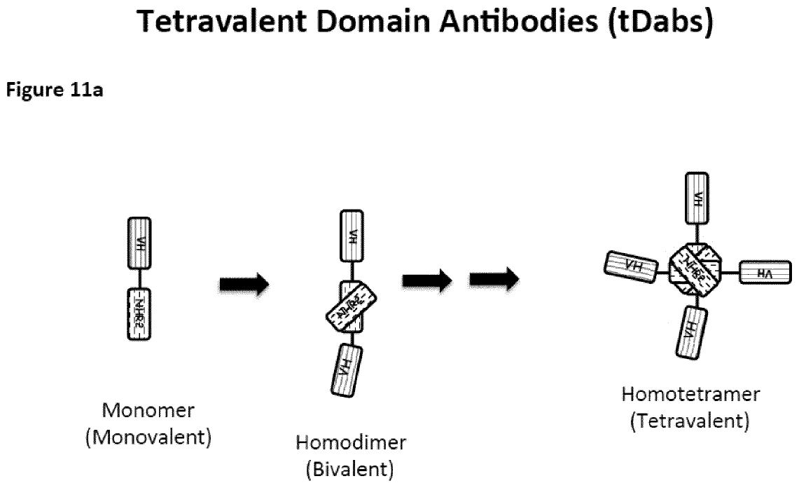

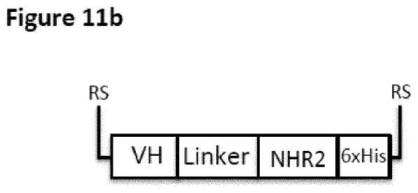

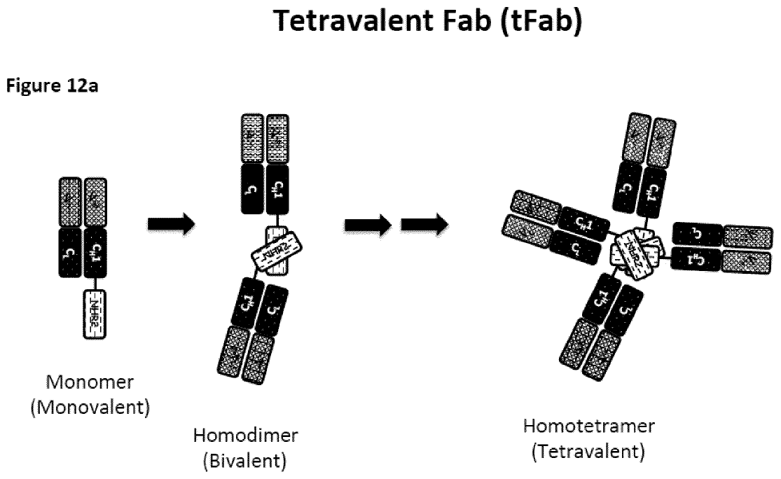

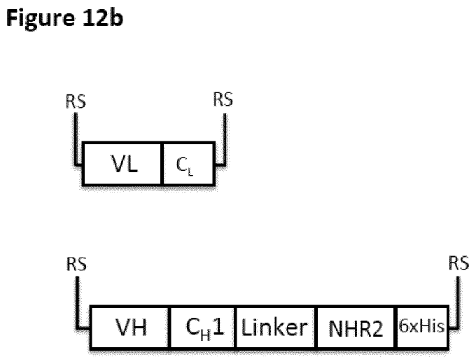

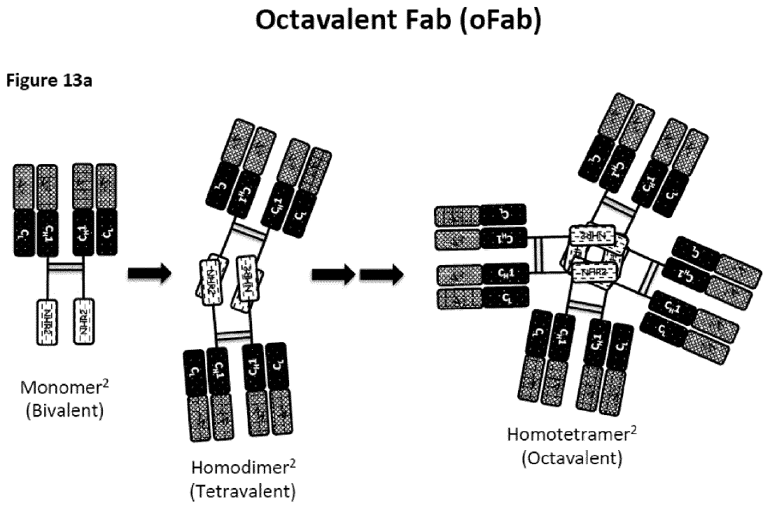

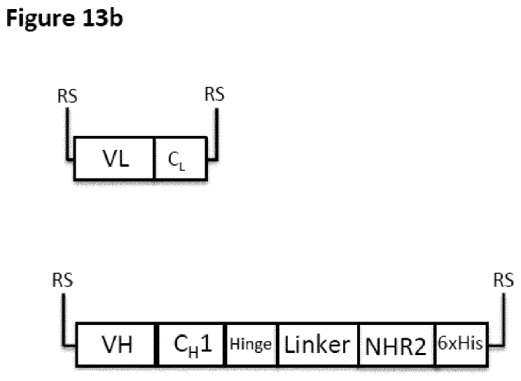

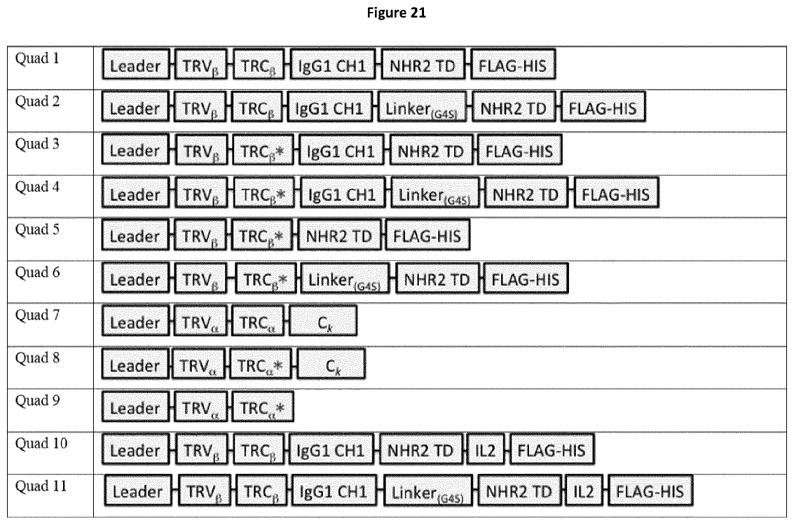

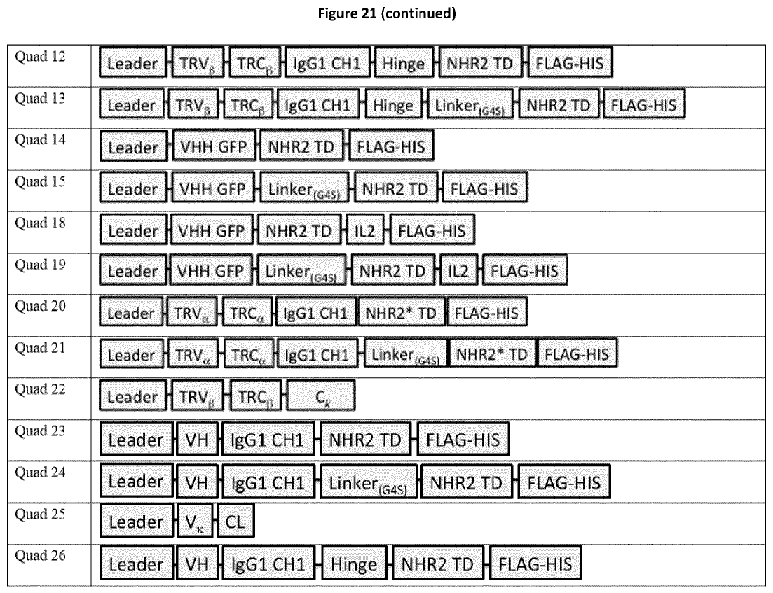

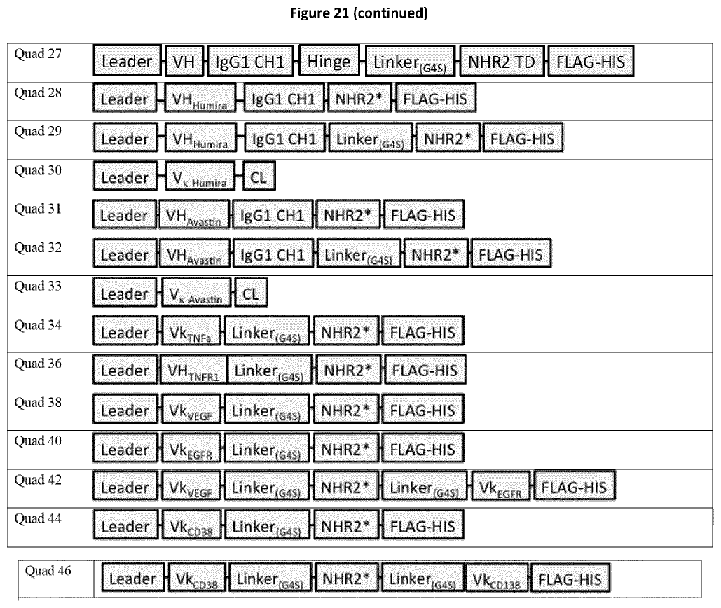

(i) A monomer as shown in FIG. 1;
(ii) A homodimer as shown in FIG. 1;
(iii) A homotetramer as shown in FIG. 1;
(iv) A monomer² as shown in FIG. 2;
(v) A homodimer² as shown in FIG. 2;
(vi) A homotetramer² as shown in FIG. 2;
(vii) A monomer as shown in FIG. 11a;
(viii) A homodimer as shown in FIG. 11a;
(ix) A homotetramer as shown in FIG. 11a;
(x) A monomer as shown in FIG. 12a;
(xi) A homodimer as shown in FIG. 12a;
(xii) A homotetramer as shown in FIG. 12a;
(xiii) A monomer² as shown in FIG. 13a;
(xiv) A homodimer² as shown in FIG. 13a;
(xv) A homotetramer² as shown in FIG. 13a; or
(xvi) A multimeric protein comprising any one of (i) to (xv) (eg, any one of Quads 3, 4, 12, 13, 14, 15, 16 and 17) or a multimer of any protein shown in FIG. 21 (excluding any leader or tag);
(xvii) A plurality of multimers of (xvi); or
(xviii) A pharmaceutical composition comprising any one of (i) to (xvii) and a pharmaceutically acceptable carrier, diluent or excipient.

The invention also provides

(i) A tetravalent or octavalent antibody V molecule;
(ii) A tetravalent or octavalent antibody Fab molecule;
(iii) A tetravalent or octavalent antibody dAb molecule;
(iv) A tetravalent or octavalent antibody scFv molecule;
(v) A tetravalent or octavalent antibody TCR V molecule; or
(vi) A tetravalent or octavalent antibody scFv molecule;
Wherein the molecule is
(a) soluble in aqueous solution (eg, a solution or cell culture medium disclosed herein) and/or;
(b) capable of being intracellularly and/or extracellularly expressed by HEK293 cells.

The invention provides a claim multimer (eg, tetramer) of NHR2 or p53 (or another TD disclosed herein) fused at its N- and/or C-terminus to an amino acid sequence (eg, a peptide, protein domain or protein) that is not an NHR2 sequence. For example, sequence is selected from a TCR (eg, TCRα, TCRβ, Cα or Cβ), cytokine (eg, interleukin, eg, IL-2, IL-12, IL-12 and IFN), antibody fragments (eg, scFv, dAb or Fab) and a antibody domain (eg, V or C domain, eg, VH, VL, Vκ, Vλ, CH, CH1, CH2, CH3, hinge, Cκ or Cλ domain). Optionally, the multimer is the molecule is

a) soluble in aqueous solution (eg, a solution or cell culture medium disclosed herein) and/or;
b) capable of being intracellularly and/or extracellularly expressed by HEK293 cells.

The invention provides:—
(i) Use of NHR2 or p53 (or another TD disclosed herein) for the manufacture of a polypeptide for soluble expression of a multimer of the polypeptide from a cell, eg, a eukaryotic cell, eg, a mammalian, HEK293, CHO or Cos cell.
(ii) Use of NHR2 or p53 (or another TD disclosed herein) for the manufacture of a polypeptide for intracellular expression of a multimer of the polypeptide in a cell, eg, a eukaryotic cell, eg, a mammalian, HEK293, CHO or Cos cell.
(iii) A cell comprising an intracellular expression product, wherein the product comprises a multimer of a polypeptide comprising NHR2 or p53 (or another TD disclosed herein) fused at its N- and/or C-terminus to an amino acid sequence (eg, a peptide, protein domain or protein) that is not an NHR2 sequence.
(iv) Use of NHR2 as a promiscuous tetramerisation domain for tetramerising peptides, protein domains, polypeptides or proteins in that manufacture of multimers that are intracellularly and/or solubly expressed from host cell.

Optionally, the amino acid is an amino acid sequence of a human peptide, protein domain or protein, eg, a TCR (eg, TCRα, TCRβ, Cα or Cβ), cytokine (eg, interleukin, eg, IL-2, IL-12, IL-12 and IFN), antibody fragments (eg, scFv, dAb or Fab), or an antibody domain (eg, V or C domain, eg, VH, VL, Vκ, Vλ, CH, CH1, CH2, CH3, hinge, Cκ or Cλ domain).

Optionally, the or each polypeptide comprises a polypeptide selected from the group consisting of Quad 1-46 (ie, a polypeptide as shown in FIG. 21 but excluding any leader or tag sequence). Optionally, the invention provides a multimer (eg, a dimer, trimer, tetramer, pentamer, hexamer, septamer or octamer, preferably a tetramer or octamer) of a polypeptide selected from the group consisting of such Quad 1-46 (ie, 2, 3, 4, 5, 6, 7 or 8 copies of such a polypeptide), eg, for medical or diagnostic use, eg, medical use for treating or preventing a disease or condition in a human or animal (eg, a human).

Optionally, the or each polypeptide comprises a polypeptide (excluding any leader or tag sequence) that is encoded by a nucleotide sequence selected from the group consisting of SEQ ID NOs: 13-50. Optionally, the or each polypeptide comprises a polypeptide (excluding any leader or tag sequence) that comprises an amino acid sequence selected from the group consisting of SEQ ID NOs: 83-115. Optionally, the invention provides a multimer (eg, a dimer, trimer, tetramer, pentamer, hexamer, septamer or octamer, preferably a tetramer or octamer) of such a polypeptide, eg, for medical or diagnostic use, eg, medical use for treating or preventing a disease or condition in a human or animal (eg, a human).

In an example, the TD is a TD comprised by any one of SEQ ID NOs: 1-9. In an example, the TD is a TD comprising SEQ ID NO: 10 or 126. In an example, the TD is encoded by SEQ ID NO: 124 or 125. In an example, the amino acid sequence of each TD is SEQ ID NO: 10 or 126 or is at least 80, 85, 90, 95, 96m 97, 98 or 99% identical to the SEQ ID NO: 10 or 126.

In an example, the TD is a TD comprising SEQ ID NO: 120 or 123. In an example, the TD is encoded by SEQ ID NO: 116 or 119. In an example, the amino acid sequence of each TD is SEQ ID NO: 120 or 123 or is at least 80, 85, 90, 95, 96m 97, 98 or 99% identical to the SEQ ID NO: 120 or 123.

Optionally, the domain or peptide comprised by the engineered polypeptide or monomer comprises an amino acid selected from SEQ ID NOs: 51-82.

High Purity Tetramers

As exemplified herein, the invention in one configuration is based on the surprising realization that tetramerisation domains (TD), eg, p53 tetramerisation domain (p53 TD), can be used to preferentially produce tetramers of effector domains over the production of lower-order structures such as dimers and monomers. This is particularly useful for secretion of tetramers is desirable yields from mammalian expression cell lines, such as CHO, HEK293 and Cos cell lines. The invention is also particularly useful for the production of tetramers no more than 200, 160, 155 or 150 kDa in size.

Thus, the invention provides the following Concepts:—

### Concepts

1. Use of a tetramerisation domain (TD) (eg, p53 tetramerisation domain (p53 TD) or NHR2 TD) or a homologue or orthologue thereof in a method of the manufacture of a tetramer of polypeptides, for producing a higher yield of tetramers versus monomer and/or dimer polypeptides.

The monomers and dimers comprise one or two copies of the TD, homologue or orthologue respectively

In an example, the TD, orthologue or homologue is a human domain.

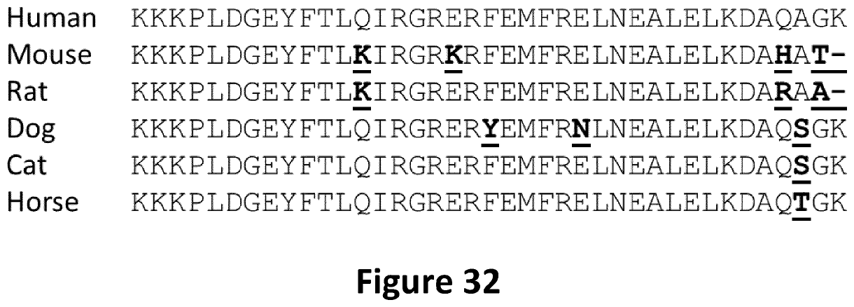

Herein, the TD is a human TD or a homologue, eg, a TD selected from any of the p53 TD sequences disclosed in UniProt (www.uniprot.org), for example the p53 TD is a TD disclosed in Table 13. In an example, the homologue is a p53TD of a non-human animal species, eg, a mouse, rat, horse cat or dog p53TD. See FIG. 32, which shows the high level of conservation between p53 TDs of different species, which supports the use of non-human p53 TDs as an alternative to human p53 TDs. In an example, the homologue is a p53TD of a non-human mammalian species. In an example, the homologue is identical to human p53 TD with the exception of up to 10, 9, 8, 7, 6, 5, 4, 3, 2 or 1 amino acid change(s).

In an example, the yield of tetramers is higher than the yield of monomers; In an example, the yield of tetramers is higher than the yield of dimers; In an example, the yield of tetramers is higher than the yield of trimers; In an example, the yield of tetramers is higher than the yield of monomers and dimers; In an example, the yield of tetramers is higher than the yield of monomers and trimers; In an example, the yield of tetramers is higher than the yield of monomers, dimers and trimers

For example, the TD is the TD of p53 isoform 1. In an example, the TD comprises or consists of an amino acid sequence that is identical to positions 325 to 356 (or 319-360; or 321-359) of human p53 (eg, isoform 1). Optionally, the TD, orthologue or homologue comprises or consists of an amino acid sequence that is at least 80, 85, 90, 95, 96, 97, 98 or 99% identical to SEQ ID NO: 10, 126, 11 or 12. For example the sequence is identical to said selected sequence. Optionally, the TD, orthologue or homologue comprises or consists of an amino acid sequence that is at least 80, 85, 90, 95, 96, 97, 98 or 99% identical to SEQ ID NO: 120, 121, 122 or 123. For example the sequence is identical to said selected sequence.

2. The use of Concept 1, wherein first, second, third and fourth copies of an identical TD or homologue or orthologue thereof is used.
3. The use of any preceding Concept, wherein the TD is a NHR2, p53, p63 or p73 tetramerisation domain.
For example, the TD is a p53 TD. In an example, the TD is an orthologue or homologue of a p53 TD, eg, a human p53 TD.
4. The use of any preceding Concept, wherein the yield of tetramers is at least 10× the yield of monomers and/or dimers.

Optionally, the yield is at least 2×3×, 4×, 5×, 6×, 7×, 8×, 9×, or 10× the yield of monomers and/or dimers. Optionally, the ratio of tetramers produced:monomers and/or dimers is at least 90:10, eg, at least 95:5; or 96:4; or 97:3; or 98:2; or 99:1. Optionally only tetramers are produced.

In an embodiment, each domain comprised by each monomer, dimer or tetramer is a human domain; and optionally the monomer, dimer or tetramer does not comprise non-human amino acid sequences or linkers.

5. The use of any preceding Concept, wherein the ratio of tetramers produced:monomers and/or dimers produced in the method is at least 90:10 (ie, 9× the amount of monomers; 9× the amount of dimers; or 9× the amount of the combination of monomers and dimers).
6. The use of Concept 4 or 5, wherein the yield or ratio is determinable or determined by obtaining a sample of the product of the tetramer manufacture method, using a protein separation technique on the sample to resolve tetramers, monomers and dimers and comparing the amount of tetramers with the amount of monomers and dimers.

Amounts of tetramers, monomers, dimers and trimers can be determined, for example, using Western Blot analysis of a gel described herein, eg, a native gel, ie, a gel not under denatured conditions, such as in the absence of SDS.

7. The use of Concept 4 or 5, wherein the yield or ratio is determinable or determined by
(a) Obtaining a sample of the product of the tetramer manufacture method;
(b) Carrying out polyacrylamide gel electrophoresis (PAGE) under non-reducing conditions to resolve the sample into a band corresponding to said tetramers and a band corresponding to said monomers and/or a band corresponding to said dimers; and
(c) Comparing the tetramer band with the monomer and/or dimer band(s) to determine said yield or ratio, eg, by comparing the relative band intensities and/or band sizes.
8. The use of Concept 4 or 5, wherein the yield or ratio is determinable or determined by
(d) Obtaining a sample of the product of the tetramer manufacture method;
(e) Carrying out polyacrylamide gel electrophoresis (PAGE) under non-reducing conditions to resolve the sample into a band corresponding to said tetramers, eg, wherein the gel is under non-denatured conditions (eg, in the absence of sodium dodecylsuphate (SDS);
(f) Determining that there is no band corresponding to said monomers and/or no band corresponding to said dimers.
9. The use of Concept 7 or 8, comprising
(g) Obtaining a second sample of the product of the tetramer manufacture method;
(h) Carrying out polyacrylamide gel electrophoresis (PAGE) under reducing conditions to resolve the second sample into a band corresponding to said monomers and/or a band corresponding to said dimers, eg, wherein the gel is under non-denatured conditions (eg, in the absence of sodium dodecylsuphate (SDS); and
(i) Comparing the gel produced by step (h) with the gel of step (b) or (e) to determine the position of monomer and/or dimer band(s) in the gel of step (b) or where such gels would be expected in the gel of step (e).
10. The use of any preceding Concept, wherein each monomer has a size of no more than 40 kDa.

For example, the monomer has a size of no more than 35, 30, 25, 24, 23, 22, 21 or 20 kDa

11. The use of any preceding Concept, wherein each tetramer has a size of no more than 150 kDa.

For example, the tetramer has a size of no more than 80, 90, 100, 110, 120, 130 or 140 kDa.

12. The use of any preceding Concept, wherein the method comprises expressing the tetramers from a mammalian cell line, eg, a HEK293, CHO or Cos cell line.

For example, the cell line is a HEK293 (eg, HEK293T) cell line. In the alternative, the cell line is a yeast (eg, Saccharomyces or Pichia, eg, P pastoris) or bacterial cell line.

13. The use of any preceding Concept, wherein the method comprises secreting the tetramers from a mammalian cell line, eg, a HEK293, CHO or Cos cell line.

Thus, advantageously in an example, the use or tetramer is for expression from a mammalian cell line (eg, a HEK293, CHO or Cos cell line) or a eukaryotic cell line. This is useful for large-scale manufacture of the products, eg, tetramers, of the invention.

For example, the cell line is a HEK293 (eg, HEK293T) cell line. In the alternative, the cell line is a yeast (eg, Saccharomyces or Pichia, eg, P pastoris) or bacterial cell line.

14. The use of any preceding Concept, wherein each polypeptide or monomer comprises a said TD, homologue or orthologue and one or more protein effector domains, such as one or more antibody domains, eg, one or more antibody domains forming an antigen binding site.
15. The use of Concept 14, wherein the polypeptide comprises one or more of
(i) an antibody single variable domain (dAb or VHH or Nanobody™) that is capable of specifically binding an antigen;
(ii) an scFv that is capable of binding an antigen or an scTCR that is capable of binding pMHC;
(iii) a Fab that is capable of binding an antigen; or
(iv) a TCR variable domain or pMHC binding site.
16. The use of any preceding Concept, wherein each polypeptide or monomer comprises a said TD, homologue or orthologue and one or more incretin, insulin, GLP-1 or Exendin-4 domains.
17. The use of any preceding Concept, wherein each polypeptide or monomer comprises a said TD, homologue or orthologue; and first and second antigen binding sites.
18. The use of Concept 17, wherein each binding site is provided by
(i) an antibody single variable domain (dAb or VHH or Nanobody™) that is capable of specifically binding an antigen;
(ii) an scFv that is capable of binding an antigen or an scTCR that is capable of binding pMHC;
(iii) a Fab that is capable of binding an antigen; or
(iv) a TCR variable domain or pMHC binding site.
19. The use of Concept 18, wherein each binding site is provided by an antibody single variable domain.
20. The use of any one of Concepts 14 to 18, wherein the TD, homologue or orthologue is directly fused to said further domain(s).
21. The use of Concept 20, wherein each monomer or polypeptide comprises the TD, homologue or orthologue fused directly or via a peptide linker to the C-terminal of a said further domain.
22. A tetramer of polypeptides, wherein each polypeptide comprises
(i) a tetramerisation domain (TD) (eg, a p53 TD or a NHR2 TD) or a homologue or orthologue thereof;
(ii) one or more protein effector domains; and
(iii) optionally a linker linking (i) to (ii) (eg, linking the C-terminus of (ii) to the N-terminus of (i));
wherein optionally each tetramer has a size of no more than 150 or 200 kDa.

For example, the tetramer has a size of no more than 80, 90, 100, 110, 120, 130 or 140 kDa. In an example, any multimer, dimer, trimer, tetramer, octamer, dodecamer, hexadecamer or 20-mer herein has a size of at least 60 or 80 kDa; this may be useful for example to increase half-life in a human or animal subject administered with the multimer, dimer, trimer, tetramer, octamer, dodecamer, hexadecamer or 20-mer (eg, to treat or prevent a disease or condition in the subject). Sizes in these ranges may be above the renal filtration size.

In an alternative, the invention provides a monomer, dimer, octamer, dodecamer, hexadecamer or 20-mer instead of a tetramer.

23. The tetramer of Concept 22, wherein each polypeptide comprises one or more of
(i) an antibody single variable domain (dAb or VHH or Nanobody™) that is capable of specifically binding an antigen;
(ii) an scFv that is capable of binding an antigen or an scTCR that is capable of binding pMHC;
(iii) a Fab that is capable of binding an antigen; or
(iv) a TCR variable domain or pMHC binding site.
24. The tetramer of Concept 22 or 23, wherein each polypeptide comprises a said TD, homologue or orthologue and one or more incretin, insulin, GLP-1 or Exendin-4 domains.
25. The tetramer of Concept 22 or 23, wherein each polypeptide comprises a said TD, homologue or orthologue; and first and second antigen binding sites.
26. The tetramer of Concept 25, wherein each binding site is provided by
(i) an antibody single variable domain (dAb or VHH or Nanobody™) that is capable of specifically binding an antigen;
(ii) an scFv that is capable of binding an antigen or an scTCR that is capable of binding pMHC;
(iii) a Fab that is capable of binding an antigen; or
(iv) a TCR variable domain or pMHC binding site.
27. The tetramer of Concept 26, wherein each binding site is provided by an antibody single variable domain.
28. The tetramer of any one of Concepts 22 to 27, wherein the TD, homologue or orthologue is directly fused to said effector domain(s).
29. The tetramer of any one of Concepts 22 to 27, wherein each polypeptide comprises the TD, homologue or orthologue fused directly or via a peptide linker to the C-terminal of a said effector domain.

In an embodiment, each polypeptide comprises only 2 (ie, only a first and a second, but not a third) effector domains or only 2 dAbs, VHH, scFvs, scTCRs, Fabs or antigen binding sites.

30. A pharmaceutical composition comprising a tetramer of any one of Concepts 22 to 29 and a pharmaceutically acceptable carrier, diluent or excipient.

Optionally the composition is comprised by a sterile medical container or device, eg, a syringe, vial, inhaler or injection device.

31. A cosmetic, foodstuff, beverage, cleaning product, detergent comprising a tetramer of any one of Concepts 22 to 29.
32. A mixture comprising a cell line (eg, a mammalian cell line, eg, a HEK293, CHO or Cos cell line) encoding a polypeptide as recited in any preceding Concept; and tetramers as defined in any preceding Concept.

Optionally, the mixture is comprised by a sterile container.

33. The mixture of Concept 32, wherein the cell line is in a medium comprising secretion products of the cells, wherein the secretion products comprise said tetramers.
34. The mixture of Concept 33, wherein the secretion products do not comprise monomers and/or dimers as defined in any one of Concepts 1 to 31.
35. The mixture of Concept 33, wherein the secretion products comprise said tetramers in an amount of at least 10× the amount of monomers and/or dimers.
36. The mixture of Concept 33, wherein the secretion products comprise said tetramers in a ratio of tetramers:monomers and/or dimers of at least 90:10.
37. A method for enhancing the yield of tetramers of an protein effector domain (eg, an antibody variable domain or binding site), the method comprising expressing from a cell line (eg, a mammalian cell, CHO, HEK293 or Cos cell line) tetramers of a polypeptide, wherein the polypeptide is as defined in any preceding Concept and comprises one or more effector domains; and optionally isolating said expressed tetramers.

The homologue, orthologue or equivalent has multimerisation or tetramerisation function.

Homologue: A gene, nucleotide or protein sequence related to a second gene, nucleotide or protein sequence by descent from a common ancestral DNA or protein sequence. The term, homologue, may apply to the relationship between genes separated by the event of or to the relationship between genes separated by the event of genetic duplication.

Orthologue: Orthologues are genes, nucleotide or protein sequences in different species that evolved from a common ancestral gene, nucleotide or protein sequence by speciation. Normally, orthologues retain the same function in the course of evolution.

In an example, the TD, orthologue or homologue is a TD of any one of proteins 1 to 119 listed in Table 2. In an example, the orthologue or homologue is an orthologue or homologue of a TD of any one of proteins 1 to 119 listed in Table 2. In an embodiment, instead of the use of a p53 tetramerisation domain (p53-TD) or a homologue or orthologue thereof, all aspects of the invention herein instead can be read to relate to the use or inclusion in a polypeptide, monomer, dimer, trimer or tetramer of aTD of any one of proteins 1 to 119 listed in Table 2 or a homologue or orthologue thereof. The TD may be a NHR2 (eg, a human NHR2) TD or an orthologue or homologue thereof. The TD may be a p63 (eg, a human p63) TD or an orthologue or homologue thereof. The TD may be a p73 (eg, a human p73) TD or an orthologue or homologue thereof. This may have one or more advantages as follows:—

secretion of tetramers from mammalian or other eukaryotic cells, eg, a mammalian cell disclosed herein such as CHO, HEK293 or Cos;
enhanced yield of secreted tetramers versus monomers;
enhanced yield of secreted tetramers versus dimers;
enhanced yield of secreted tetramers versus trimers;
enhanced yield of secreted tetramers versus monomers and dimers combined;
enhanced yield of secreted tetramers versus monomers, dimers and trimers combined;
enhanced affinity or avidity of antigen binding in tetramers comprising antigen binding sites;
enhanced tetramer production and/or expression, wherein the tetramer is no more than 200 or no more than 160 or 150 kDa in size.

In an embodiment, each polypeptide or monomer comprises one or more VH, VL or VH/VL binding sites of an antibody selected from ReoPro™; Abciximab; Rituxan™; Rituximab; Zenapax™; Daclizumab; Simulect™; Basiliximab; Synagis™; Palivizumab; Remicade™; Infliximab; Herceptin™; Trastuzumab; Mylotarg™; Gemtuzumab; Campath™; Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara™; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; Kadcyla™; Ado-trastuzumab; Gazyva™ and Obinutuzumab. In an alternative, (eg, for treating or preventing a cancer in a human) each polypeptide or monomer comprise one or more VH, VL or VH/VL binding sites of an antibody selected from ipilimumab (or YERVOY™), tremelimumab, nivolumab (or OPDIVO™), pembrolizumab (or KEYTRUDA™), pidilizumab, BMS-936559, durvalumab and atezolizumab. Optionally, the binding site of the polypeptide of the multimer comprises a VH of the binding site of the antibody and also the CH1 of the antibody (ie, in N- to C-terminal direction the VH-CH1 and SAM). In an embodiment, the polypeptide may be paired with a further polypeptide comprising (in N- to C-terminal direction a VL-CL, eg, wherein the CL is the CL of the antibody).

In an example, the tetramer comprises 4 copies of the antigen binding site of a first antibody selected from the group consisting of ipilimumab (or YERVOY™), tremelimumab, nivolumab (or OPDIVO™), pembrolizumab (or KEYTRUDA™), pidilizumab, BMS-936559, durvalumab and atezolizumab and optionally 4 copies of the antigen binding site of a second antibody selected from said group, wherein the first and second antibodies are different. For example, the first antibody is ipilimumab (or YERVOY™) and optionally the second antibody is nivolumab (or OPDIVO™) or pembrolizumab (or KEYTRUDA™). This is useful for treating or preventing a cancer in a human.

In an example, the tetramer comprises 4 copies of the antigen binding site of Avastin. In an example, the tetramer comprises 4 copies of the antigen binding site of Humira. In an example, the tetramer comprises 4 copies of the antigen binding site of Erbitux. In an example, the tetramer comprises 4 copies of the antigen binding site of Actemra™. In an example, the tetramer comprises 4 copies of the antigen binding site of sarilumab. In an example, the tetramer comprises 4 copies of the antigen binding site of dupilumab. In an example, the tetramer comprises 4 copies of the antigen binding site of alirocumab or evolocumab. In an example, the tetramer comprises 4 copies of the antigen binding site of In an example, the tetramer comprises 4 copies of the antigen binding site of Remicade. In an example, the tetramer comprises 4 copies of the antigen binding site of Lucentis. In an example, the tetramer comprises 4 copies of the antigen binding site of Eylea™. Such tetramers are useful for administering to a human to treat or prevent a cancer. Such tetramers are useful for administering to a human to treat or prevent an ocular condition (eg, wet AMD or diabetic retinopathy, eg, when the binding site is an Avastin, Lucentis or Eylea site). Such tetramers are useful for administering to a human to treat or prevent angiogenesis.

In an example, the tetramer comprises 4 copies of insulin. In an example, the tetramer comprises 4 copies of GLP-1. In an example, the tetramer comprises 4 copies of GIP. In an example, the tetramer comprises 4 copies of Exendin-4. In an example, the tetramer comprises 4 copies of insulin and 4 copies of GLP-1. In an example, the tetramer comprises 4 copies of insulin and 4 copies of GIP. In an example, the tetramer comprises 4 copies of insulin and 4 copies of Exendin-4. In an example, the tetramer comprises 4 copies of GLP-1 and 4 copies of Exendin-4. Such tetramers are useful for administering to a human to treat or prevent diabetes (eg, Type II diabetes) or obesity.

### Example Antigens

The polypeptide, multimer may bind to one or more antigens or epitopes, or each of the binding sites herein (eg, dAb or scFv binding sites) herein may bind to an antigen or epitope. In an example, an (or each) antigen herein is selected from the following list. In an example, an (or each) epitope herein is an epitope of an antigen selected from the following list.

Activin type-II receptor; Activin type-IIB receptor; ADAM11; ADAM12; ADAM15; ADAM17; ADAM18; ADAM19; ADAM1A; ADAM1B; ADAM2; ADAM20; ADAM21; ADAM22; ADAM23; ADAM24P; ADAM28; ADAM29; ADAM30; ADAM32; ADAM33; ADAM3A; ADAM3B; ADAM5; ADAM6; ADAM7; ADAM8; ADAM9; ADORA2A; AKT; ALK; alpa-4 integrin; alpha synuclein; anthrax protective antigen; BACE1; BCMA; beta amyloid; BRAF; BTLA; BTNL2; CCR4; CCR5; CD126; CD151; CD16; CD160; CD19; CD20; CD22; CD226; CD244; CD27; CD274 (PDL1); CD276; CD28; CD3; CD30; CD300A; CD300C; CD300E; CD300LB; CD300LF; CD33; CD38; CD3; CD3 epsilon; CD3 delta; CD3 gamma; CD40; CD40L; CD47; CD48; CD5; CD52; CD59; CD6; CD70; CD72; CD73; CD80; CD81; CD84; CD86; CD96; CDK4/6; CEA; CEACAM1; CEACAM3; CGRP receptor; CLEC12A; CLEC1B; CLEC4A; CLEC5A; CLEC7A; Clostridium difficile toxin; cMET; Complement C5 factor; Complement factor D; CSF1R; CSF2RA; CTAG1B; CTLA4; CXCL12; CXCR2; CXCR4; DR4; DR5; EDA; EDA2R; EGFR; EGFRvIII; EMR1; ENTPD1; EpCAM; Factor IX; Factor X; Factor VII; FAP; FAS; FCAR; FCER1G; FCER2; TFR2; 4-1BB; FCGR2A; FCGR2B; FCGR3A; FCGR3B; FCRL1; FCRL3; FCRL4; FCRL6; FGRF1/2/3; FLT3; GAL; GEM; GITR; GITRL; GM-CSF; GM-CSF receptor; GP IIb IIIa; gpNMB; TIM3; HDAC1; HER-2; HER3; HFE; HHLA2; Histone H1 modulator; HLA-C; HLA-G; HMGB1; HMMR; HVEM; ICAM-1; ICOS; ICOSL; IDO1; IFNG; IL-1 beta; IL-12; IL-13; IL-2; IL-22R; IL-23; IL-23a; IL-24; IL-2R; IL-34; IL-8; IL10; IL11; IL13; IL17A; IL17D; IL22; IL2RA; IL36G; IL4; IL4a; IL5; IL6; Immunoglobulin E; Immunoglobulin E; Immunoglobulin G; INHBA; INHBB; Interferon type I receptor; INF-a-2a/2b; INF-b-la/lb; ITGA2B; ITGB3; KIR; KIR2DL1; KIR2DL2; KIR2DL3; KIR2DL4; KIR2DL5A; KIR3DL1; KIR3DL3; KIR3DS1; KIT; KLRC1; KLRC2; KLRF1; KLRG1; KLRK1; KRAS; LAG3; LAIR1; LAIR2; LFA-1; LIGHT; LILRA1; LILRA2; LILRA3; LILRA4; LILRA5; LILRA6; LILRB1; LILRB2; LILRB3; LILRB4; LILRB5; LILRP1; LILRP2; LTA; LTBR; LY9; MadCam; MAGE-C1; MAGE-C2; MARCO; MEK-1/2; MIA3; MIC; MICA; MICB; MMP9; MS4A1; MS4A2; mTOR; MUC1; MUCIN-1; Nav1.7; Nav1.8; NCR1; NCR2; NCR3; NGF; NGFR; NT5E; NY-ESO-1; OX40; OX40L; p53; PARP; PCSK9; PD-1; PDCD1LG2; PDCD6; PDGF receptor alpha; PECAM1; PI3K delta; PILRA; PPP1R1B; PSMA; PTPN6; PVR; PVRL2; PVRL3; RANKL; Respiratory syncytial virus protein; SIGLEC10; SIGLEC12; SIGLEC15; SIGLEC5; SIGLEC6; SIGLEC7; SIGLEC9; SIRPA; SIRPB1; SLAMF1; SLAMF6; SLAMF7; SLAMF8; SNCA; SOD1; STAT3; STING; SURVIVIN; TARM1; Tau; TDP43; TfR1; TGF-b; TGM2; TIGIT; TIM-3; TLR-4; TLR03; TMEM30a; TMIGD2; TNFa; TNFRSF10A; TNFRSF10B; TNFRSF10C; TNFRSF10D; TNFRSF11A; TNFRSF11B; TNFRSF12A; TNFRSF13B; TNFRSF13C; TNFRSF14; TNFRSF17; TNFRSF18; TNFRSF19; TNFRSF21; TNFRSF4; TNFRSF6B; TNFRSF8; TNFRSF9; TNFSF10; TNFSF11; TNFSF12; TNFSF13; TNFSF13B; TNFSF14; TNFSF15; TNFSF18; TNFSF4; TNFSF8; TNFSF9; Transmembrane glycoprotein NMB modulator; TREML1; TREML2; TSLP; VEGF; VEGF-2R; VEGF1; VEGFA; VEGFL; VEGFR; Viral envelope glycoprotein; Viral protein haemagglutinin; VISTA; VSIG4; VSTM1; VTCN1; and WEE-1. In an example, an antigen herein is a PCSK9, eg, human PCSK9; optionally the multimer has 4, 8, 12 or 16 copies an anti-PCSK9 binding site (eg, a dAbs).

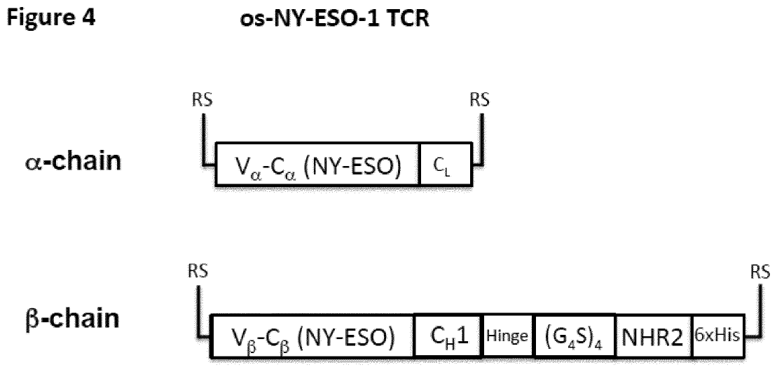

An example antigen is a toxin, such as a snake venom toxin, eg, wherein a multimer of the invention is administered (such as systemically or by IV injection) to a human or animal subject and the antigen binding sites comprised by the multimer specifically bind to the toxin in the subject. Preferably, each binding site or domain of the multimer is a dAb (eg, a Nanobody™). For example, each snake venom toxin antigen binding site of the multimer of the invention is a C33 single domain VH as disclosed in FIG. 4 of PLoS One. 2013 Jul. 22; 8(7):e69495. doi: 10.1371/journal.pone.0069495; “In vivo neutralization of α-cobratoxin with high-affinity llama single-domain antibodies (VHHs) and a VHH-Fc antibody”, Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C15 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C7 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C13 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C19 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C34 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C31 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C20 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C2 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C29 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C42 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. For example, each snake venom toxin antigen binding site of the multimer of the invention is a C43 single domain VH as disclosed in FIG. 4 of Richard et al, the amino acid of which as disclosed in said FIG. 4 is incorporated herein in its entirety by reference for possible use in the present invention as a binding site or domain or dAb or Nanobody or VHH or VH. An example of a snake venom toxin is 3FTx, dendrotoxin or PLA2 toxin. Optionally, the toxin is an alpha-neurotoxin, eg, from Cobra.

Another example of a toxin is a blood toxin, eg, wherein a multimer of the invention is administered (such as systemically or by IV injection) to a human or animal subject and the antigen binding sites comprised by the multimer specifically bind to the toxin in the blood of the subject. These examples are useful for sequestering the toxin or for reducing the toxic effect of the toxin to the subject or to promote excretion or metabolism of the toxin.

In an example, the antigen is a viral antigen, each a capsid protein or carbohydrate (eg, a sugar). In an example, a multimer of the invention binds to a virus or virus antigen, eg, a virus selected from Table 19 wherein the virus comprises a surface antigen that is bound by the multimer; or the multimer of the invention binds to a cell or virus antigen, eg, selected from an antigen disclosed in Table 20. Binding to the virus may, for example, reduce or inhibit attachment of the virus to its host cell or infection of the cell by the virus. For example, the invention provides a method of treating or preventing (eg, reducing the risk of) a viral or cell infection in a human or animal or plant subject (eg, in a human subject), the method comprising administering a multimer of the invention to the subject wherein the multimer binds to a surface antigen of the virus, thereby inhibiting the virus from attaching to a host cell; inhibiting infection of a host cell by the virus and/or sequestering the virus in the subject (eg, to mark the bound virus for clearance from the systemic circulation or a tissue of the subject). In an alternative, For example, the invention provides a method of treating or preventing (eg, reducing the risk of) a bacterial or archaeal cell infection in a human or animal or plant subject (eg, in a human subject), the method comprising administering a multimer of the invention to the subject wherein the multimer binds to a surface antigen of the cell, thereby inhibiting infection of the subject by the cell and/or sequestering the cell in the subject (eg, to mark the bound cell for clearance from the systemic circulation or a tissue of the subject). In an alternative, For example, the invention provides a method of treating or preventing (eg, reducing the risk of) a cancer in a human or animal subject (eg, in a human subject), the method comprising administering a multimer of the invention to the subject wherein the multimer binds to a surface antigen of a tumour cell, thereby sequestering the cell in the subject (eg, to mark the bound cell for clearance from the systemic circulation or a tissue of the subject) or marking the cell for targeting by the immune system or another therapy (eg, immune checkpoint therapy or CAR-T therapy) administered to the subject.

In an example, the antigen is selected from CXCR2, CXCR4, GM-CSF, ICAM-1, IFN-g, IL-1, IL-10, IL-12, IL-1R1, IL-1R2, IL-1Ra, IL-1β, IL-4, IL-6, IL-8, MIF, TGF-β, TNF-α, TNFR1, TNFR2 and VCAM-1. Targeting one or more of these antigens may be useful for treating or preventing sepsis in a subject. Thus, in an example the multimer of the invention comprises one or more antigen binding sites (eg, each one provided by a dAb), wherein the multimer is for use in a method of treating or preventing sepsis in a human or animal subject, wherein the multimer is administered to the subject (eg, systemically or intravenously). Optionally, the multimer is monospecific, bispecific, trispecific or tetraspecific for antigen binding. For example, the multimer is bispecific, trispecific or tetraspecific for an antigen selected from CXCR2, CXCR4, GM-CSF, ICAM-1, IFN-g, IL-1, IL-10, IL-12, IL-1R1, IL-1R2, IL-1Ra, IL-1β, IL-4, IL-6, IL-8, MIF, TGF-β, TNF-α, TNFR1, TNFR2 and VCAM-1. There is also provided a pharmaceutical composition comprising such a multimer and a pharmaceutically acceptable diluent, carrier or excipient. There is also provided a method of treating or preventing sepsis in a human or animal subject, the method comprising administering the multimer to the subject, eg, systemically or intravenously.

### Diseases and Conditions

The polypeptide monomer or multimer (eg, dimer, trimer, tetramer or octamer) of the invention can be used in a method for administration to a human or animal subject to treat or prevent a disease or condition in the subject.

Optionally, the disease or condition is selected from

(a) A neurodegenerative disease or condition;
(b) A brain disease or condition;
(c) A CNS disease or condition;
(d) Memory loss or impairment;
(e) A heart or cardiovascular disease or condition, eg, heart attack, stroke or atrial fibrillation;
(f) A liver disease or condition;
(g) A kidney disease or condition, eg, chronic kidney disease (CKD);
(h) A pancreas disease or condition;
(i) A lung disease or condition, eg, cystic fibrosis or COPD;
(j) A gastrointestinal disease or condition;
(k) A throat or oral cavity disease or condition;
(l) An ocular disease or condition;
(m) A genital disease or condition, eg, a vaginal, labial, penile or scrotal disease or condition;
(n) A sexually-transmissible disease or condition, eg, gonorrhea, HIV infection, syphilis or Chlamydia infection;
(o) An ear disease or condition;
(p) A skin disease or condition;
(q) A heart disease or condition;
(r) A nasal disease or condition
(s) A haematological disease or condition, eg, anaemia, eg, anaemia of chronic disease or cancer;
(t) A viral infection;
(u) A pathogenic bacterial infection;
(v) A cancer;
(w) An autoimmune disease or condition, eg, SLE;
(x) An inflammatory disease or condition, eg, rheumatoid arthritis, psoriasis, eczema, asthma, ulcerative colitis, colitis, Crohn's disease or IBD;

### (y) Autism;

### (z) ADHD;

(aa) Bipolar disorder;

### (bb) ALS [Amyotrophic Lateral Sclerosis];

### (cc) Osteoarthritis;

(dd) A congenital or development defect or condition;

### (ee) Miscarriage;

(ff) A blood clotting condition;

### (gg) Bronchitis;

### (hh) Dry or wet AMD;

(ii) Neovascularisation (eg, of a tumour or in the eye);
(jj) Common cold;

### (kk) Epilepsy;

(ll) Fibrosis, eg, liver or lung fibrosis;
(mm) A fungal disease or condition, eg, thrush;
(nn) A metabolic disease or condition, eg, obesity, anorexia, diabetes, Type I or Type II diabetes.
(oo) Ulcer(s), eg, gastric ulceration or skin ulceration;
(pp) Dry skin;
(qq) Sjogren's syndrome;
(rr) Cytokine storm;
(ss) Deafness, hearing loss or impairment;
(tt) Slow or fast metabolism (ie, slower or faster than average for the weight, sex and age of the subject);
(uu) Conception disorder, eg, infertility or low fertility;

### (vv) Jaundice;

(ww) Skin rash;

### (xx) Kawasaki Disease;

### (yy) Lyme Disease;

(zz) An allergy, eg, a nut, grass, pollen, dust mite, cat or dog fur or dander allergy;
(aaa) Malaria, typhoid fever, tuberculosis or cholera;
(bbb) Depression;
(ccc) Mental retardation;
(ddd) Microcephaly;
(eee) Malnutrition;
(fff) Conjunctivitis;
(ggg) Pneumonia;
(hhh) Pulmonary embolism;
(iii) Pulmonary hypertension;
(jj) A bone disorder;
(kkk) Sepsis or septic shock;
(lll) Sinusitus;
(mmm) Stress (eg, occupational stress);
(nnn) Thalassaemia, anaemia, von Willebrand Disease, or haemophilia;
(ooo) Shingles or cold sore;
(ppp) Menstruation;
(qqq) Low sperm count.

### Neurodegenerative or CNS Diseases or Conditions for Treatment or Prevention

In an example, the neurodegenerative or CNS disease or condition is selected from the group consisting of Alzheimer disease, geriopsychosis, Down syndrome, Parkinson's disease, Creutzfeldt-jakob disease, diabetic neuropathy, Parkinson syndrome, Huntington's disease, Machado-Joseph disease, amyotrophic lateral sclerosis, diabetic neuropathy, and Creutzfeldt Creutzfeldt-Jakob disease. For example, the disease is Alzheimer disease. For example, the disease is Parkinson syndrome.

In an example, wherein the method of the invention is practised on a human or animal subject for treating a CNS or neurodegenerative disease or condition, the method causes downregulation of Treg cells in the subject, thereby promoting entry of systemic monocyte-derived macrophages and/or Treg cells across the choroid plexus into the brain of the subject, whereby the disease or condition (eg, Alzheimer's disease) is treated, prevented or progression thereof is reduced. In an embodiment the method causes an increase of IFN-gamma in the CNS system (eg, in the brain and/or CSF) of the subject. In an example, the method restores nerve fibre and/or reduces the progression of nerve fibre damage. In an example, the method restores nerve myelin and/or reduces the progression of nerve myelin damage. In an example, the method of the invention treats or prevents a disease or condition disclosed in WO2015136541 and/or the method can be used with any method disclosed in WO2015136541 (the disclosure of this document is incorporated by reference herein in its entirety, eg, for providing disclosure of such methods, diseases, conditions and potential therapeutic agents that can be administered to the subject for effecting treatment and/or prevention of CNS and neurodegenerative diseases and conditions, eg, agents such as immune checkpoint inhibitors, eg, anti-PD-1, anti-PD-L1, anti-TIM3 or other antibodies disclosed therein).

### Cancers for Treatment or Prevention

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and paediatric tumours/cancers are also included.

Haematologic cancers are cancers of the blood or bone marrow. Examples of haematological (or haematogenous) cancers include leukaemias, including acute leukaemias (such as acute lymphocytic leukaemia, acute myelocytic leukaemia, acute myelogenous leukaemia and myeloblasts, promyeiocytic, myelomonocytic, monocytic and erythroleukaemia), chronic leukaemias (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel!carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, medu!loblastoma, Schwannoma craniopharyogioma, ependymoma, pineaioma, hemangioblastoma, acoustic neuroma, oligodendroglioma, menangioma, neuroblastoma, retinoblastoma and brain metastases).

Autoimmune Diseases for Treatment or Prevention
Acute Disseminated Encephalomyelitis (ADEM)
Acute necrotizing hemorrhagic leukoencephalitis
Addison's disease
Agammaglobulinemia
Alopecia areata
Amyloidosis
Ankylosing spondylitis
Anti-GBM/Anti-TBM nephritis
Antiphospholipid syndrome (APS)
Autoimmune angioedema
Autoimmune aplastic anemia
Autoimmune dysautonomia
Autoimmune hepatitis
Autoimmune hyperlipidemia
Autoimmune immunodeficiency
Autoimmune inner ear disease (AIED)
Autoimmune myocarditis
Autoimmune oophoritis
Autoimmune pancreatitis
Autoimmune retinopathy
Autoimmune thrombocytopenic purpura (ATP)
Autoimmune thyroid disease
Autoimmune urticaria
Axonal & neuronal neuropathies
Balo disease
Behcet's disease
Bullous pemphigoid
Cardiomyopathy
Castleman disease
Celiac disease
Chagas disease
Chronic fatigue syndrome
Chronic inflammatory demyelinating polyneuropathy (CIDP)
Chronic recurrent multifocal ostomyelitis (CRMO)
Churg-Strauss syndrome
Cicatricial pemphigoid/benign mucosal pemphigoid
Crohn's disease
Cogans syndrome
Cold agglutinin disease
Congenital heart block
Coxsackie myocarditis
CREST disease
Essential mixed cryoglobulinemia
Demyelinating neuropathies
Dermatitis herpetiformis
Dermatomyositis
Devic's disease (neuromyelitis optica)
Discoid lupus
Dressler's syndrome
Endometriosis
Eosinophilic esophagitis
Eosinophilic fasciitis
Erythema nodosum
Experimental allergic encephalomyelitis
Evans syndrome
Fibromyalgia
Fibrosing alveolitis
Giant cell arteritis (temporal arteritis)
Giant cell myocarditis
Glomerulonephritis
Goodpasture's syndrome
Granulomatosis with Polyangiitis (GPA) (formerly called Wegener's Granulomatosis)
Graves' disease
Guillain-Barre syndrome
Hashimoto's encephalitis
Hashimoto's thyroiditis
Hemolytic anemia
Henoch-Schonlein purpura
Herpes gestationis
Hypogammaglobulinemia
Idiopathic thrombocytopenic purpura (ITP)
IgA nephropathy
IgG4-related sclerosing disease
Immunoregulatory lipoproteins
Inclusion body myositis
Interstitial cystitis
Juvenile arthritis
Juvenile diabetes (Type 1 diabetes)
Juvenile myositis
Kawasaki syndrome
Lambert-Eaton syndrome
Leukocytoclastic vasculitis
Lichen planus
Lichen sclerosus
Ligneous conjunctivitis
Linear IgA disease (LAD)
Lupus (SLE)
Lyme disease, chronic
Meniere's disease
Microscopic polyangiitis
Mixed connective tissue disease (MCTD)
Mooren's ulcer
Mucha-Habermann disease
Multiple sclerosis
Myasthenia gravis
Myositis
Narcolepsy
Neuromyelitis optica (Devic's)
Neutropenia
Ocular cicatricial pemphigoid
Optic neuritis
Palindromic rheumatism
PANDAS (Pediatric Autoimmune Neuropsychiatric Disorders Associated with Streptococcus)
Paraneoplastic cerebellar degeneration
Paroxysmal nocturnal hemoglobinuria (PNH)
Parry Romberg syndrome
Parsonnage-Turner syndrome
Pars planitis (peripheral uveitis)
Pemphigus
Peripheral neuropathy
Perivenous encephalomyelitis
Pernicious anemia
POEMS syndrome
Polyarteritis nodosa
Type I, II, & III autoimmune polyglandular syndromes
Polymyalgia rheumatica
Polymyositis
Postmyocardial infarction syndrome
Postpericardiotomy syndrome
Progesterone dermatitis
Primary biliary cirrhosis
Primary sclerosing cholangitis
Psoriasis
Psoriatic arthritis
Idiopathic pulmonary fibrosis
Pyoderma gangrenosum
Pure red cell aplasia
Raynauds phenomenon
Reactive Arthritis
Reflex sympathetic dystrophy
Reiter's syndrome
Relapsing polychondritis
Restless legs syndrome
Retroperitoneal fibrosis
Rheumatic fever
Rheumatoid arthritis
Sarcoidosis
Schmidt syndrome
Scleritis
Scleroderma
Sjogren's syndrome
Sperm & testicular autoimmunity
Stiff person syndrome
Subacute bacterial endocarditis (SBE)
Susac's syndrome
Sympathetic ophthalmia
Takayasu's arteritis
Temporal arteritis/Giant cell arteritis
Thrombocytopenic purpura (TTP)
Tolosa-Hunt syndrome
Transverse myelitis
Type 1 diabetes
Ulcerative colitis
Undifferentiated connective tissue disease (UCTD)
Uveitis
Vasculitis
Vesiculobullous dermatosis
Vitiligo
Wegener's granulomatosis (now termed Granulomatosis with Polyangiitis (GPA).

Inflammatory Diseases for Treatment or Prevention
Alzheimer's
ankylosing spondylitis
arthritis (osteoarthritis, rheumatoid arthritis (RA), psoriatic arthritis)
asthma
atherosclerosis
Crohn's disease
colitis
dermatitis
diverticulitis
fibromyalgia
hepatitis
irritable bowel syndrome (IBS)
systemic lupus erythematous (SLE)
nephritis
Parkinson's disease
ulcerative colitis.

### Multivalent Soluble TCR

The present configuration relates to a multivalent soluble TCR protein. In one aspect, the invention relates to tetravalent and octavalent soluble TCR analogues. The TCR proteins of the invention are capable of self-assembly from monomers and are entirely of human origin. The proteins are multimers which comprise an ETO NHR2 multimerisation domain. The invention also relates to methods of constructing multimeric soluble TCRs, and methods of using such proteins.

Attempts to exploit alternative soluble TCR formats as therapeutic molecules have lagged far behind compared to the plethora of antibody formats. This is largely due to TCR, a heterodimeric transmembrane protein having the intrinsic problem of solubility once the extracellular TCR/s chains are dissociated from their transmembrane and cytoplasmic domain. Secondly the intrinsic low affinity and avidity of these molecules for their cognate ligand at the target site has to a large degree hampered their development as a therapeutic molecule.

In order to overcome these drawbacks, the present configuration of the invention provides a TCR protein which is both multivalent and soluble. Multivalency increases the avidity of the TCR for cognate pMHC, and solubility allows the TCR to be used outside of a transmembrane environment. Accordingly, in a first aspect there is provided a multivalent heterodimeric soluble T cell receptor capable of binding pMHC complex comprising:

(i) TCR extracellular domains;
(ii) (ii) immunoglobulin constant domains; and
(iii) (iii) an NHR2 multimerisation domain of ETO.

The use of Ig constant domains provides the TCR extracellular domains with stability and solubility; multimerisation via the NHR2 domains provides multivalency and increased avidity. Advantageously, all of the domains are of human origin or conform to human protein sequences.

Using the Ig constant domain to stabilise and render soluble the TCR avoids the use of non-native disulphide bonds. Advantageously, therefore, the TCR of the invention does not comprise a non-native disulphide bond.

In one embodiment, said complex comprises a heavy chain and a light chain, and each light chain comprises a TCR Vα domain and an immunoglobulin C_{α} domain, and each heavy chain comprises a TCR Vβ domain and an immunoglobulin C_{H}1 domain.

In one embodiment, each light chain additionally comprises a TCR Cα domain, and each heavy chain additionally comprises a TCR Cβ domain.

In embodiments, the TCR and immunoglobulin domains can be separated by a flexible linker.

The NHR2 multimerisation domain is advantageously attached to the C-terminus of an immunoglobulin domain. Thus, each dimer of heavy and light chains will be attached to one multimerisation domain, so that the heavy chain-light chain dimers associate into multivalent oligomers.

In embodiments, the multimerisation domain and the immunoglobulin domain are separated by a flexible linker. In certain embodiments, this allows the multimerisation domain to multimerise without hindrance from the immunoglobulin domain(s).

In embodiments, the TCR protein may further comprise an immunoglobulin hinge domain. Hinge domains allow dimerization of heavy chain-light chain dimers; this allows further multimerisation of the TCR proteins. For example, a multimerisation domain which forms polypeptide tetramers can, using an immunoglobulin hinge domain, form multimers up to polypeptide octamers. Likewise, a dimerising multimerisation domain can form tetramers in the presence of a hinge domain.

In embodiments, the TCR protein of the invention is tetravalent.

In embodiments, the TCR protein of the invention is octavalent

The present invention provides a soluble TCR where it is stably assembled in a tetravalent heterodimeric format using the nervy homology region 2 (NHR2) domain found in the ETO family protein in humans (Liu et al. 2006). The NHR2 domain is found naturally to form homotetramer, which is formed from pairing of two NHR2 homodimers. NHR2 linked operably to the extracellular TCRα or TCRβ chain will preferentially form tetravalent heterodimeric soluble TCR protein molecules sequentially self-assembled from a monomer followed by a homodimer (FIG. 1).

TCR proteins assembling into octamers can be created using the NHR2 domain, by employing immunoglobulin hinge domains.

In a further aspect, the TCR proteins of the invention can be coupled to biologically active polypeptides/effector molecules. Examples of such polypeptides can include immunologically active moieties such as cytokines, binding proteins such as antibodies or targeted polypeptides, and the like.

The invention further relates to methods for making tetravalent and octavalent heterodimeric soluble TCR, the DNA vectors encoding the proteins used for transfecting host cells of interests and the use of these novel highly sensitive multivalent soluble TCR protein molecules. Applications for use could include but not limited to, therapeutics, diagnostics and drug discovery.

In a further aspect, the invention provides a method for constructing multivalent immunoglobulin molecules in an efficient manner, without employing non-human construct components.

Accordingly, there is provided a multimeric immunoglobulin comprising

(i) immunoglobulin variable domains; and
(ii) an NHR2 multimerisation domain of ETO.

The immunoglobulin variable domains are preferably antibody variable domains. Such domains are fused to the ETO NHR2 multimerisation domain, which provides means for forming tetramers of the immunoglobulin variable domains.

The ETO NHR2 domain is more efficient than p53 and similar multimerisation domains in the production of immunoglobulin multimers, and permits the production of multimeric immunoglobulin molecules without the use of non-human components in the construct.

Also provided is a method for producing a multimeric immunoglobulin, comprising expressing immunoglobulin variable domains in fusion with an NHR2 domain of ETO, and allowing the variable domains to assemble into multimers.

Preferably, the immunoglobulin variable domains are attached to one or more immunoglobulin constant domains.

Advantageously, the immunoglobulin domains are antibody domains. For example, the variable domains can be V_{H} and V_{L} antibody domains. For example, the constant domains are antibody CH1 domains.

In one embodiment, the multimeric immunoglobulin molecules according to the invention, both TCR and non-TCR immunoglobulins, are produced for screening by phage display or another display technology. For example, therefore, the multivalent immunoglobulins are produced as fusions with a phage coat protein. For each immunoglobulin produced fused to a coat protein, other immunoglobulin molecules are produced without a coat protein, such that they can assemble on the phage surface as a result of NHR2 multimerisation.

The present configuration of the invention as detailed above relates to the nucleic acid sequences and methods for producing novel multivalent, for example tetravalent and octavalent, soluble proteins. In one aspect in particular the soluble protein is a TCR assembled into a tetravalent heterodimeric format that can bind four pMHC with high sensitivity, affinity and specificity. The soluble tetravalent heterodimeric TCR is a unique protein molecule composed from either the entire or in part the extracellular TCR α/β chains. The extracellular TCR α/β chains are linked to immunoglobulin C_{H}1 and C_{L} (either Cκ or Cλ) domains. This linkage allows stable formation of heterodimeric TCR α/β. In the context of soluble tetravalent TCR the unique feature is the NHR2 homotetramer domain of the ETO family of proteins, which is operably linked to the C-terminus of C_{H}1 or the C-terminus of C_{L}. Linkage of the NHR2 domain to the heterodimeric α/βTCR in this manner allows it to self-assemble into a tetravalent format inside cells and be subsequently secreted into the supernatant as a soluble protein.

### TCR Extracellular Domains

TCR extracellular domains are composed of variable and constant regions. These domains are present in T-cell receptors in the same way as they are present in antibodies and other immunoglobulin domains. The TCR repertoire has extensive diversity created by the same gene rearrangement mechanisms used in antibody heavy and light chain genes (Tonegawa, S. (1988) Biosci. Rep. 8:3-26). Most of the diversity is generated at the junctions of variable (V) and joining (J) (or diversity, D) regions that encode the complementarity determining region 3 (CDR3) of the α and β chains (Davis and Bjorkman (1988) Nature 334:395-402). Databases of TCR genes are available, such as the IMGT LIGM database, and methods for cloning TCRs are known in the art—for example, see Bentley and Mariuzza (1996) Ann. Rev. Immunol. 14:563-590; Moysey et al., Anal Biochem. 2004 Mar. 15; 326(2):284-6; Wälchli, et al. (2011) A Practical Approach to T-Cell Receptor Cloning and Expression. PLoS ONE 6(11): e27930.

### Immunoglobulin Variable Domains

Antibody variable domains are known in the art and available from a wide variety of sources. Databases of sequences of antibody variable domains exist, such as IMGT and Kabat, and variable domains can be produced by cloning and expression of natural sequences, or synthesis of artificial nucleic acids according to established techniques.

Methods for the construction of bacteriophage antibody display libraries and lambda phage expression libraries are well known in the art (McCafferty et al. (1990) Nature. 348: 552; Kang et al. (1991) Proc. Natl. Acad. Sci. USA., 88: 4363; Clackson et al. (1991) Nature. 352: 624; Lowman et al. (1991) Biochemistry, 30: 10832; Burton et al. (1991) Proc. Natl. Acad Sci USA., 88: 10134; Hoogenboom et al. (1991) Nucleic Acids Res., 19: 4133; Chang et al. (1991) J Immunol., 147: 3610; Breitling et al. (1991) Gene. 104: 147; Marks et al. (1991) supra; Barbas et al. (1992) supra; Hawkins and Winter (1992) J Immunol., 22: 867; Marks et al., 1992, J Bioi. Chem., 267: 16007; Lerner et al. (1992) Science, 258: 1313, incorporated herein by reference).

One particularly advantageous approach has been the use of scFv phage-libraries (Huston et al., 1988, Proc. Natl. Acad. Sci U.S.A., 85: 5879-5883; Chaudhary et al. (1990) Proc. Natl. Acad. Sci U.S.A., 87: 1066-1070; McCafferty et al. (1990) supra; Clackson et al. (1991) Nature. 352: 624; Marks et al. (1991) J Mol. Bioi., 222: 581; Chiswell et al. (1992) Trends Biotech., 10: 80; Marks et al. (1992) J Bioi. Chem., 267). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference.

Such techniques can be adapted for the production of multimeric immunoglobulins by the fusion of NHR2 multimerisation domains to the antibody variable domains

### Immunoglobulin Constant Domains

An immunoglobulin constant domain, as referred to herein, is preferably an antibody constant domain. Constant domains do vary in sequence between antibody subtypes; preferably, the constant domains are IgG constant domains. Preferably, the constant domains are CH1 constant domains. Antibody constant domains are well known in the art and available from a number of sources and databases, including the IMGT and Kabat databases.

The fusion of antibody constant domains to immunoglobulin variable domains is also known in the art, for example in the construction of engineered Fab antibody fragments.

### Linkers

Flexible linkers can be used to connect TCR variable domain—Ig constant domain to the NHR2 multimerisation domain. This allows the TCR domains and the multimerisation domain to function without steric hindrance from each other or other molecules in the multimeric complex. Suitable linkers comprise, for example, glycine repeats, glycine-alanine repeats, Gly(4)Ser linkers, or flexible polypeptide linkers as set forth in Reddy Chichili et al., 2012 Protein Science 22:153-167.

### Immunoglobulin Hinge Domain

The Ig Hinge domain, herein preferably an antibody hinge domain, is the domain which links antibody constant regions in a natural antibody. This domain therefore provides for natural dimerization of molecules which include an antibody constant domain. It is present, for example, in a F(ab)2 antibody fragment, as well as whole antibodies such as IgG. This region comprises two natural interchain disulphide bonds, which connect the two CH1 constant domains together.

The multimerisation domain, in one embodiment, may be attached to the Ig constant domain or to the hinge domain. If a hinge domain is present, the multimerisation domain will form a TRC octamer, comprising four dimers of TCR variable-Ig Constant domains joined at a hinge region. Without the hinge region, the multimerisation domain will lead to the formation of a tetramer. Preferably, the multimerisation domain is attached to the C-terminal end of the constant domain or the hinge region.

### Biologically Active Molecule

One or more biologically active molecules or effector molecules (EM) can be attached to the multimer, eg, multimeric TCR proteins, of the present invention. Such molecules may be, for example, antibodies, especially antibodies which may assist in immune recognition and functioning of the TCR, such as anti-CD3 antibodies or antibody fragments.

In some aspects, the biologically active molecule can be a cytotoxic drug, toxin or a biologically active molecule such as a cytokine, as described in more detail below. Examples of biologically active molecules include chemokines such as MIP-lb, cytokines such as IL-2, growth factors such as GM-CSF or G-CSF, toxins such as ricin, cytotoxic agents, such as doxorubicin or taxanes, labels including radioactive and fluorescent labels, and the like. For examples of biologically active molecules conjugatable to TCRs, see US20110071919.

In other aspects, the biologically active molecule is, for example, selected from the group consisting of: a group capable of binding to a molecule which extends the half-life of the polypeptide ligand in vivo, and a molecule which extends the half-life of the polypeptide ligand in vivo. Such a molecule can be, for instance, HSA or a cell matrix protein, and the group capable of binding to a molecule which extends the half-life of the TCR molecule in vivo is an antibody or antibody fragment specific for HSA or a cell matrix protein.

In one embodiment, the biologically active molecule is a binding molecule, for example an antibody fragment. 2, 3, 4, 5 or more antibody fragments may be joined together using suitable linkers. The specificities of any two or more of these antibody fragments may be the same or different; if they are the same, a multivalent binding structure will be formed, which has increased avidity for the target compared to univalent antibody fragments.

The biologically active molecule can moreover be an effector group, for example an antibody Fc region.

Attachments to the N or C terminus may be made prior to assembly of the TCR molecule or engineered polypeptide into multimers, or afterwards. Thus, the TCR fusion with an Ig Constant domain may be produced (synthetically, or by expression of nucleic acid) with an N or C terminal biologically active molecule already in place. In certain aspects, however, the addition to the N or C terminus takes place after the TCR fusion has been produced. For example, Fluorenylmethyloxycarbonyl chloride can be used to introduce the Fmoc protective group at the N-terminus of the TCR fusion. Fmoc binds to serum albumins including HSA with high affinity, and Fmoc-Trp or FMOC-Lys bind with an increased affinity. The peptide can be synthesised with the Fmoc protecting group left on, and then coupled with the scaffold through the cysteines. An alternative is the palmitoyl moiety which also binds HSA and has, for example been used in Liraglutide to extend the half-life of this GLP-1 analogue.

Alternatively, the TCR fusion can be modified at the N-terminus, for example with the amine- and sulfhydryl-reactive linker N-e-maleimidocaproyloxy)succinimide ester (EMCS). Via this linker the TCR can be linked to other polypeptides, for example an antibody Fc fragment.

### The NHR2 Domain

AML1/ETO is the fusion protein resulting from the t(8; 21) found in acute myeloid leukemia (AML) of the M2 subtype. AML1/ETO contains the N-terminal 177 amino acids of RUNX1 fused in frame with most (575 aa) of ETO. The nervy homology domain 2 of ETO is responsible for many of the biological activities associated with AML1/ETO, including oligomerisation and protein-protein interactions. This domain is characterised in detail in Liu et al (2006). See Genbank accession number NG_023272.2.

In one aspect of the present invention, the protein assembled into a soluble multivalent format is a TCR composed of either in part or all of the extracellular domains of the TCR α and β chains. The TCR α and β chains are stabilized by immunoglobulin C_{H}1 and C_{L} domains and could be arranged in the following configurations:

1. Vα-C_{L} and VβC_{H}1
2. Vα-C_{H}1 and Vβ-C_{L}
3. VαCα-C_{L} and VβCβ-C_{H}1
4. VαCαC_{H}1 and VβCβC_{L}

In one aspect of this invention, the extracellular TCR domains are linked to immunoglobulin C_{H}1 and C_{L} domains via an optional peptide linker (L) to promote protein flexibility and facilitate optimal protein folding.

1. Vα-(L)-C_{L} and Vβ-(L)-C_{H}1
2. Vα-(L)-C_{H}1 and Vβ-(L)-C_{L}
3. VαCα-(L)-C_{L} and VβCβ-(L)-C_{H}1
4. VαCα-(L)-C_{H}1 and VβCβ-(L)-C_{L}

In another aspect of this invention, a tetramerisation domain (TD) such as NHR2 homotetramer domain is linked to the C-terminus of either the immunoglobulin C_{H}1 or C_{L} domain, which is linked to the extracellular TCR α and β chain. The NHR2 domain could be optionally linked to C_{H}1 or C_{L} domain via a peptide linker. The resulting tetravalent heterodimeric TCR protein could be arranged in the following configurations where (L) is an optional peptide linker:

1. Vα-(L)-C_{L} and Vβ-(L)-C_{H}1-(L)-TD
2. Vα-(L)-C_{H}1-(L)-TD and Vβ-(L)-C_{L}
3. VαCα-(L)-C_{L} and VβCβ-(L)-C_{H}1-(L)-TD
4. VαCα-(L)-C_{H}1-(L)-TD and VβCβ-(L)-C_{L}
5. Vα-(L)-C_{L}-(L)-TD and Vβ-(L)-C_{H}1
6. Vα-(L)-C_{H}1 and Vβ-(L)-C_{L}-(L)-TD
7. VαCα-(L)-C_{L}-(L)-TD and VβCβ-(L)-C_{H}1
8. VαCα-(L)-C_{H}1 and VβCβ-(L)-C_{L}-(L)-TD

The sensitivity of the soluble TCR for its cognate pMHC can be enhanced by increasing the avidity effect. This is achieved by increasing the number of antigen binding sites, facilitated by the tetramerisation domain. This in turn also increases the molecular weight of the protein molecule compared to a monovalent soluble TCR and thus extends serum retention in circulation. Increasing the serum half-life also enhances the likelihood of these molecules interacting with their cognate target antigens.

The tetravalent heterodimeric soluble TCR protein molecule is capable of binding simultaneously to one, two, three or four pMHC displayed on a single cell or bind simultaneously to one, two, three or four different cells displaying its cognate pMHC.

TCR α and β chain sequences used in this invention could be from a known TCR specific for a particular pMHC or identified de novo by screening using techniques known in the art, such as phage display. Furthermore, TCR sequences are not limited to α and β chain in this invention but can also incorporate TCRδ and γ or ε chain and sequence variations thereof either directly cloned from human T cells or identified by directed evolution using recombinant DNA technology.

In another aspect to this invention, the tetravalent heterodimeric soluble TCR protein molecules are preferentially produced in mammalian cells for optimal production of soluble, stable and correctly folded protein molecules.

Multimer (eg, tetramer or octamer), or multivalent TCR according to the present invention may be expressed in cells, such as mammalian cells, using any suitable vector system. The pTT5 expression vector is one example of an expression system is used to express multivalent soluble TCR. The pTT5 expression system allows for high-level transient production of recombinant proteins in suspension-adapted HEK293 EBNA cells (Zhang et al. 2009). It contains origin of replication (oriP) that is recognized by the viral protein Epstein-Barr Nuclear Antigen 1 (EBNA-1), which together with the host cell replication factor mediates episomal replication of the DNA plasmid allowing enhanced expression of recombinant protein. Other suitable vector system for mammalian cell expression known in the art and commercially available can be used with this invention.

The tetravalent heterodimeric soluble TCR protein molecules or other multimers can be produced by transiently expressing genes from an expression vector.

In another embodiment, tetravalent heterodimeric soluble TCR protein molecules or other multimers can be produced from an engineered stable cell line. Cell lines can be engineered to produce the protein molecule using genome-engineering techniques known in the art where the gene(s) encoding for the protein molecule is integrated into the genome of the host cells either as a single copy or multiple copies. The site of DNA integration can be a defined location within the host genome or randomly integrated to yield maximum expression of the desired protein molecule. Genome engineering techniques could include but not limited to, homologous recombination, transposon mediated gene transfer such as PiggyBac transposon system, site specific recombinases including recombinase-mediated cassette exchange, endonuclease mediated gene targeting such as CRISPR/Cas9, TALENs, Zinc-finger nuclease, meganuclease and virus mediated gene transfer such as Lentivirus.

Also, in another aspect to the invention, the tetravalent heterodimeric soluble TCR protein molecule or other multimer is produced by overexpression in the cytoplasm of E. coli as inclusion bodies and refolded in vitro after purification by affinity chromatography to produce functional protein molecules capable of correctly binding to its cognate pMHC or antigen.

In another aspect to the invention, expression of the tetravalent heterodimeric soluble TCR protein molecule or other multimer is not limited to mammalian or bacterial cells but can also be expressed and produced in insect cells, plant cells and lower eukaryotic cells such as yeast cells.

In another aspect to this invention, the heterodimeric soluble TCR molecule or other multimer is produced as an octavalent protein complex, eg, having up to eight binding sites for its cognate pMHC (FIG. 2). The multiple antigen binding sites allow this molecule to bind up to eight pMHC displayed on one cell or bind pMHC displayed on up to eight different cells thus creating a highly sensitive soluble TCR.

The heterodimeric soluble TCR portion of the molecule is made into a bivalent molecule by fusing the immunoglobulin hinge domain to the C-terminus of either the C_{H}1 or C_{L}domain, which is linked itself either to TCR α or β chain. The hinge domain allows for the connection of two heavy chains giving a structure similar to IgG. To the C-terminus of the hinge domain, a tetramerisation domain such as NHR2 is linked via an optional peptide linker. By joining immunoglobulin hinge to C- and N-terminus of Ig CH1 or CL domain and NHR2 domain respectively, it allows for the assembly of two NHR2 monomers referred to as monomer². In this conformation we predict the two NHR2 domains will most likely not form a homodimer by an antiparallel association due to structural constraints unless a long flexible linker is provided between the hinge and NHR2 domain. Linkage of the tetramerisation and the hinge domain to the to the heterodimeric soluble TCR via immunoglobulin C_{H}1 or C_{L} domain allows for the stepwise self-assembly of an octavalent soluble TCR formed through a NHR2 homotetramer². The self-assembly of the octavalent soluble TCR is via NHR2 monomer² and homodimer² intermediate protein complexes (FIG. 2). The resulting octavalent heterodimeric soluble TCR protein molecule will have superior sensitivity for its cognate pMHC thus giving it a distinctive advantage of identifying unknown antigen or pMHC without having to affinity mature the TCR for its pMHC ligand much beyond affinities seen naturally. In particular it would be useful for identifying pMHC recognized by uncharacterized tumour-specific T cells and T cells involved in other diseases such as autoimmune diseases. A number of different configurations of the octavalent heterodimeric soluble TCR protein molecules can be produced. Some examples are shown below.

1. Vα-(L)-C_{L} and Vβ-(L)-C_{H}1-Hinge-(L)-TD
2. Vα-(L)-C_{H}1-Hinge-(L)-TD and Vβ-(L)-C_{L}
3. Vα-Cα-(L)-C_{L} and Vβ-Cβ-(L)-C_{H}1-Hinge-(L)-TD
4. VαCα(L)-C_{H}1-Hinge-(L)-TD and VβCβ-(L)-C_{L}
5. Vα-(L)-C_{L}-(L)-TD and Vβ-(L)-C_{H}1-Hinge
6. Vα-(L)-C_{H}1-Hinge and Vβ-(L)-C_{L}-(L)-TD
7. Vα-Cα-(L)-C_{L}-(L)-TD and Vβ-Cβ(L)-C_{H}1-Hinge
8. Vα-Cα-(L)-C_{H}1-Hinge and Vβ-Cβ-(L)-C_{L}-(L)-TD

In another aspect to this invention, the self-assembled multivalent protein preferentially tetravalent and octavalent heterodimeric soluble TCR are fused or conjugated to biologically active agent/effector molecule thus allowing these molecules to be guided to the desired cell population such as cancers cells and exert their therapeutic effect specifically. The tumour targeting ability of monoclonal antibodies to guide an effector molecule such as a cytotoxic drug, toxins or a biologically active molecule such as cytokines is well established (Perez et al. 2014; Young et al. 2014). In a similar manner the multivalent soluble TCR molecules outlined in this invention can also be fused with effector proteins and polypeptide or conjugated to cytotoxic agents. Examples of effector protein molecules suitable for use as a fusion protein with the multivalent protein complexes outlined in this invention include but are not limited to, IFNα, IFNβ, IFNγ, IL-2, IL-11, IL-13, granulocyte colony-stimulating factor [G-CSF], granulocyte-macrophage colony-stimulating factor [GM-CSF], and tumor necrosis factor [TNF]α, IL-7, IL-10, IL-12, IL-15, IL-21, CD40L, and TRAIL, the costimulatory ligand is B7.1 or B7.2, the chemokines DC-CK1, SDF-1, fractalkine, lyphotactin, IP-10, Mig, MCAF, MlP-1α, MIP-1/3, IL-8, NAP-2, PF-4, and RANTES or an active fragment thereof. Examples of toxic agent suitable for use as a fusion protein or conjugated to the multivalent protein complexes described in this invention include but not limited to, toxins such as diphtheria toxin, ricin, Pseudomonas exotoxin, cytotoxic drugs such as auristatin, maytansines, calicheamicin, anthracyclines, duocarmycins, pyrrolobenzodiazepines. The cytotoxic drug can be conjugated by a select linker, which is either non-cleavable or cleavable by protease or is acid-labile.

To eliminate heterogeneity and improve conjugate stability the cytotoxic drug can be conjugated in a site-specific manner. By engineering specific cysteine residues or using enzymatic conjugation through glycotransferases and transglutaminases can achieve this (Panowski et al. 2014).

In another aspect of the invention, the multivalent protein complex is covalently linked to molecules allowing detection, such as fluorescent, radioactive or electron transfer agents.

In another aspect of the invention, an effector molecule (EM) is fused to the multivalent protein complex via the C-terminus of the tetramerisation domain such as NHR2 via an optional peptide linker. Fusion via the NHR2 domain can be arranged to produce multivalent protein complexes in a number of different configurations. Examples of some of the protein configurations that can be produced using the tetravalent heterodimeric soluble TCR is shown below:

1. Vα-(L)-C_{L} and Vβ-(L)-C_{H}1-(L)-TD-(L)-EM
2. Vα-(L)-C_{H}1-(L)-TD-(L)-EM and Vβ-(L)-C_{L}
3. Vα-Cα-(L)-C_{L} and Vβ-Cβ-(L)-C_{H}1-(L)-TD-(L)-EM
4. Vα-Cα-(L)-C_{H}1-(L)-TD-(L)-EM and Vβ-Cβ-(L)-C_{L}
5. Vα-(L)-C_{L}-(L)-TD-(L)-EM and Vβ-(L)-C_{H}1
6. Vα-(L)-C_{H}1 and Vβ-(L)-C_{L}-(L)-TD-(L)-EM
7. Vα-Cα-(L)-C_{L}-(L)-TD-(L)-EM and Vβ-Cβ-(L)-C_{H}1
8. Vα-Cα-(L)-C_{H}1 and Vβ-Cβ-(L)-C_{L}-(L)-TD-(L)-EM

In another aspect of the invention, the effector molecule (EM) is fused to the multivalent protein complex at the C-terminus of either the immunoglobulin CH1 or CL1 domain via an optional peptide linker. Fusion of the EM via the immunoglobulin domain can be arranged to produce multivalent protein complexes in a number of different configurations. Examples of some of the protein configurations that can be produced using the tetravalent heterodimeric soluble TCR is shown below:

9. Vα-(L)-C_{L}-(L)-EM and Vβ-(L)-C_{H}1-(L)-TD
10. Vα-(L)-C_{H}1-(L)-TD and Vβ-(L)-C_{L}-(L)-EM
11. Vα-Cα-(L)-C_{L}-(L)-EM and Vαβ-Cβ-(L)-C_{H}1-(L)-TD
12. Vα-Cα-(L)-C_{H}1-(L)-TD and Vβ-Cβ-(L)-C_{L}-(L)-EM
13. Vα-(L)-C_{L}-(L)-TD and Vβ-(L)-C_{H}1-(L)-EM
14. Vα-(L)-C_{H}1-(L)-EM and Vβ-(L)-C_{L}-(L)-TD
15. Vα-Cα-(L)-C_{L}-(L)-TD and Vβ-Cβ-(L)-C_{H}1-(L)-EM
16. Vα-Cα-(L)-C_{H}1-(L)-EM and Vβ-Cβ-(L)-C_{L}-(L)-TD

In another aspect of the invention, effector molecules (EM) are fused to the multivalent protein complex at the C-terminus of either the immunoglobulin CH1 or CL1 domain and also the C-terminus of the tetramerisation domain (e.g. NHR2) via an optional peptide linkers. This approach allows for the fusion of two effector molecules to be fused per TCR heterodimer complex. Fusion of the EM via the immunoglobulin domain and the tetramerisation domain can be arranged to produce multivalent protein complexes in a number of different configurations. Examples of some of the protein configurations that can be produced using the tetravalent heterodimeric soluble TCR is shown below:

17. Vα-(L)-C_{L}-(L)-EM and Vβ-(L)-C_{H}1-(L)-TD-(L)-EM
18. Vα-(L)-C_{H}1-(L)-TD-(L)-EM and Vβ-(L)-C_{L}-(L)-EM
19. Vα-Cα-(L)-C_{L}-(L)-EM and VβCβ-(L)-C_{H}1-(L)-TD-(L)-EM
20. Vα-Cα-(L)-C_{H}1-(L)-TD-(L)-EM and Vβ-Cβ-(L)-C_{L}-(L)-EM
21. Vα-(L)-C_{L}-(L)-TD-(L)-EM and Vβ-(L)-C_{H}1-(L)-EM
22. Vα-(L)-C_{H}1-(L)-EM and Vβ-(L)-C_{L}-(L-)TD-(L)-EM
23. Vα-Cα-(L)-C_{L}-(L)-TD-(L)-EM and Vβ-Cβ-(L)-C_{H}1-(L)-EM
24. Vα-(L)-C_{H}1-(L)-EM and Vβ-Cβ-(L)-C_{L}-(L)-TD-(L)-EM

In another aspect of the invention, the multivalent protein complex is fused to a protein tag to facilitate purification. Purification tags are known in the art and they include, without being limited to, the following tags: His, GST, TEV, MBP, Strep, FLAG.

### Non-TCR Multimers

The present invention provides a unique method for assembling proteins in a soluble multivalent format with potential to bind multiple interacting domains or antigens. The protein can be a monomer, homodimer, heterodimer or oligomer preferentially involved either directly or indirectly in the immune system, or having the potential to regulate immune responses. Examples include, but not limited to, TCR, peptide MHC class I and class II, antibodies or antigen-binding portions thereof and binding proteins having alternative non-antibody protein scaffolds.

In another aspect of the invention, the interacting domains or antigens could be any cell surface expressed or secreted proteins, peptide-associated with MHC Class I or II or any proteins associated with pathogens including viral and bacterial proteins.

Non-TCR multimers may be multimers of antibodies or antibody fragments, such as dAbs of Fabs. Examples of dAbs and Fabs in accordance with the invention include the following:

Examples of multivalent dAbs

### 25. VH-(L)-NHR2

### 26. VL(λ or κ)-(L)-NHR2

### 27. VH-(L)-NHR2-(L)-EM

### 28. VL(λ or κ)-(L)-NHR2-(L)-EM

### 29. VH-CH1-(L)-NHR2

### 30. VL(λ or κ)-CL-(L)-NHR2

### 31. VH-CH1-(L)-NHR2-(L)-EM

### 32. VL(λ or κ)-CL-(L)-NHR2-(L)-EM

Examples of multivalent Fabs

### 33. VH-CH1-(L)-NHR2 and VL(λ or κ)-CL

### 34. VL(λ or κ)-CL-(L)-NHR2 and VH-CH1

### 35. VH-CH1-Hinge-(L)-NHR2 and VL(λ or κ)-CL

### 36. VL(λ or κ)-CL-Hinge-(L)-NHR2 and VH-CH1

### 37. VH-CH1-(L)-NHR2-(L)-EM and VL(λ or κ)-CL

### 38. VL(λ or κ)-CL-(L)-NHR2-(L)-EM and VH-CH1

### 39. VH-CH1-Hinge-(L)-NHR2-(L)-EM and VL(λ or κ)-CL

### 40. VL(λ or κ)-CL-Hinge-(L)-NHR2-(L)-EM and VH-CH1

### 41. VH-CH1-(L)-NHR2 and VL(λ or κ)-CL-(L)-EM

### 42. VL(λ or κ)-CL-(L)-NHR2 and VH-CH1-(L)-EM

### 43. VH-CH1-Hinge-(L)-NHR2 and VL(λ or κ)-CL-(L)-EM

### 44. VL(λ or κ)-CL-Hinge-(L)-NHR2 and VH-CH1-(L)-EM

In the examples above, (L) denotes an optional peptide linker, whilst EM denotes a biologically active agent or effector molecule such as toxins, drugs or cytokines, and including binding molecules such as antibodies, Fabs and ScFv.

The variable light chain can be either Vλ or Vκ.

In one aspect of the invention, the assembled tetramerized protein molecule in one example could be a human pMHC for the application in drug discovery using animal drug discovery platforms (e.g. mice, rats, rabbits, chicken). In such a context, the tetramerisation domain is preferentially expressed and produced from genes originating from the animal species it is intended for. One example of such drug discovery applications would be the use of the tetramerized human pMHC as an antigen for immunization in rats for example. Once rats are immunized with pMHC the immune response is directed specifically towards the human pMHC and not the tetramerisation domain of the protein complex.

Multivalent antibodies can be produced, for example using single domain antibody sequences, fused to the NHR2 multimerisation domain.

In a related aspect to the invention, the tetravalent protein can be a peptide used as a probe for molecular imaging of tumour antigens. The multivalent binding of such a probe will have distinctive advantage over monovalent molecular probes as it will have enhanced affinity, avidity and retention time in vivo and this in turn will enhance in vivo tumour targeting.

The multimerisation domain is the NHR2 domain set forth above. Preferably, polypeptides are stabilized and/or rendered soluble by the use of Ig constant domains fused to the polypeptides, such that the fusions provide tetramers of polypeptides. Ig hinge domains can be used to provide octamers.

### Uses of Multimers

Multimeric TCR proteins according to the invention are useful in any application in which soluble TCR proteins are indicated. Particular advantages of the TCR proteins of the invention include increased avidity for the selected target, and/or the ability to bind a plurality of targets.

Thus, in one aspect, the multivalent heterodimeric soluble TCR protein molecules of the invention can be used for selectively inhibiting immune responses, for example suppression of an autoimmune response. The multivalent, for example tetravalent, nature of these soluble protein molecules gives it exquisite sensitivity and binding affinity to compete antigen-specific interactions between T cells and antigen presenting cells. This kind of neutralization effect can be therapeutically beneficial in autoimmune diseases such as rheumatoid arthritis, systemic lupus erythematosus, psoriasis, inflammatory bowel diseases, graves disease, vasculitis and type 1 diabetes.

Similarly, the tetravalent heterodimeric soluble TCR protein molecules can be used to prevent tissue transplant rejection by selectively suppressing T cell recognition of specific transplantation antigen and self antigens binding to target molecule and thus inhibiting cell-to-cell interaction.

In another aspect of the invention, the tetravalent heterodimeric soluble TCR protein molecules can be used in clinical studies such as toxicity, infectious disease studies, neurological studies, behavior and cognition studies, reproduction, genetics and xenotransplantation studies.

The tetravalent heterodimeric soluble TCR protein molecules with enhanced sensitivity for cognate pMHC can be used for the purpose of diagnostics using biological samples obtained directly from human patients. The enhanced sensitivity of the tetravalent heterodimeric soluble TCRs allows detection of potential disease-associated peptides displayed on MHC, which are naturally found to be expressed at low density. These molecules can also be used for patient stratification for enrolling patient onto relevant clinical trials.

In another aspect of the invention, octavalent heterodimeric soluble TCR protein molecules can be used in pharmaceutical preparations for the treatment of various diseases.

In another related aspect to this invention, octavalent heterodimeric soluble TCR protein molecules can be used as a probe for tumour molecular imaging or prepared as a therapeutic protein.

Optionally, the polypeptide (first polypeptide) comprises or consists of a polypeptide disclosed in Table 8. In an example the invention provides a multimer (eg, a dimer, trimer or tetramer, preferably a tetramer) of such a polypeptide. In an example, in Table 8, the multimerization domain (SAM) is a p53 domain (eg, a human p53 domain). In an example, in Table 8, the multimerization domain (SAM) is an orthologue or homologue of a p53 domain (eg, a human p53 domain).

Optionally, the invention provides a polypeptide (eg, said polypeptide or said first polypeptide), wherein the polypeptide comprises or consists of (in N- to C-terminal direction*);

A. A dAb and a self-associating multimersiation domain (SAM)
B. A first dAb, a SAM and a second dAb
C. A first scFv and a SAM;
D. A first scFv, a SAM and a second scFv;
E. A first scFv, a SAM and a first dAb;
F. A first dAb, a CH2, a CH3 and a SAM;
G. A first scFv, a CH2, a CH3 and a SAM;

### H. A VH, a CH1, a CH2 a CH3 and a SAM;

### I. A VL, a CL, a CH2, a CH3 and a SAM;

J. A dAb; a SAM, a CH2 and a CH3;
K. A scFv; a SAM, a CH2 and a CH3;

### L. A VH, a CH1, a SAM, a CH2 and a CH3;

### M. A VL, a CL, a SAM, a CH2 and a CH3;

N. A first dAb, a second dAb and a SAM;

### O. A VH, a CH1 and a SAM;

### P. A VL, a CL and a SAM;

Q. A VH, a CH1, a SAM and a first dAb;
R. A VL, a CL, a SAM and a first dAb;
S. A first dAb, a second dAb, a SAM and a third dAb;
T. A first dAb, a second dAb, a SAM and a first scFv;
U. A first dAb, a second dAb, a SAM, a third dAb and a fourth dAb;
V. A first dAb, a CH2, a CH3, a SAM and a second dAb;
W. A first dAb, a CH2, a CH3, a SAM and a first scFv;
X. A first dAb, a second dAb, a CH2, a CH3 and a SAM;
Y. A first dAb, a second dAb, a CH2, a CH3, a SAM and a third dAb;
Z. A first dAb, a second dAb, a CH2, a CH3, a SAM and a first scFv; or
AA. A first dAb, a second dAb, a CH2, a CH3, a SAM, a third dAb and a fourth dAb.

*In an alternative the components are written in the C- to N-terminal direction.

In an embodiment, polypeptide H, L, O or Q is associated with a second polypeptide, wherein the second polypeptide comprises (in N- to C-terminal direction) VL and CL, wherein the CL is associated with the CH1 of the first polypeptide.

In an embodiment, polypeptide I, M, P or R is associated with a second polypeptide, wherein the second polypeptide comprises (in N- to C-terminal direction) VH and CH1, wherein the CH1 is associated with the CL of the first polypeptide.

In an example, the polypeptide is encoded by a nucleotide sequence disclosed in Table 9. In an example, the polypeptide comprises or consists of an amino acid sequence disclosed in Table 10.

In an example (i) the polypeptide comprises (in N- to C-terminal direction);

A. A dAb and a self-associating multimersiation domain (SAM)
B. A first dAb, a SAM and a second dAb
C. A first scFv and a SAM;
D. A first scFv, a SAM and a second scFv;
E. A first scFv, a SAM and a first dAb;
F. A first dAb, a CH2, a CH3 and a SAM;
G. A first scFv, a CH2, a CH3 and a SAM;

### H. A VH, a CH1, a CH2 a CH3 and a SAM;

### I. A VL, a CL, a CH2, a CH3 and a SAM;

J. A dAb; a SAM, a CH2 and a CH3;
K. A scFv; a SAM, a CH2 and a CH3;

### L. A VH, a CH1, a SAM, a CH2 and a CH3;

### M. A VL, a CL, a SAM, a CH2 and a CH3;

N. A first dAb, a second dAb and a SAM;

### O. A VH, a CH1 and a SAM;

### P. A VL, a CL and a SAM;

Q. A VH, a CH1, a SAM and a first dAb;
R. A VL, a CL, a SAM and a first dAb;
S. A first dAb, a second dAb, a SAM and a third dAb;
T. A first dAb, a second dAb, a SAM and a first scFv;
U. A first dAb, a second dAb, a SAM, a third dAb and a fourth dAb;
V. A first dAb, a CH2, a CH3, a SAM and a second dAb;
W. A first dAb, a CH2, a CH3, a SAM and a first scFv;
X. A first dAb, a second dAb, a CH2, a CH3 and a SAM;
Y. A first dAb, a second dAb, a CH2, a CH3, a SAM and a third dAb;
Z. A first dAb, a second dAb, a CH2, a CH3, a SAM and a first scFv; or
AA. A first dAb, a second dAb, a CH2, a CH3, a SAM, a third dAb and a fourth dAb.
Or (ii) the polypeptide comprises (in C- to N-terminal direction);
A. A dAb and a self-associating multimersiation domain (SAM)
B. A first dAb, a SAM and a second dAb
C. A first scFv and a SAM;
D. A first scFv, a SAM and a second scFv;
E. A first scFv, a SAM and a first dAb;
F. A first dAb, a CH2, a CH3 and a SAM;
G. A first scFv, a CH2, a CH3 and a SAM;

### H. A VH, a CH1, a CH2 a CH3 and a SAM;

### I. A VL, a CL, a CH2, a CH3 and a SAM;

J. A dAb; a SAM, a CH2 and a CH3;
K. A scFv; a SAM, a CH2 and a CH3;

### L. A VH, a CH1, a SAM, a CH2 and a CH3;

### M. A VL, a CL, a SAM, a CH2 and a CH3;

N. A first dAb, a second dAb and a SAM;

### O. A VH, a CH1 and a SAM;

### P. A VL, a CL and a SAM;

Q. A VH, a CH1, a SAM and a first dAb;
R. A VL, a CL, a SAM and a first dAb;
S. A first dAb, a second dAb, a SAM and a third dAb;
T. A first dAb, a second dAb, a SAM and a first scFv;
U. A first dAb, a second dAb, a SAM, a third dAb and a fourth dAb;
V. A first dAb, a CH2, a CH3, a SAM and a second dAb;
W. A first dAb, a CH2, a CH3, a SAM and a first scFv;
X. A first dAb, a second dAb, a CH2, a CH3 and a SAM;
Y. A first dAb, a second dAb, a CH2, a CH3, a SAM and a third dAb;
Z. A first dAb, a second dAb, a CH2, a CH3, a SAM and a first scFv; or
AA. A first dAb, a second dAb, a CH2, a CH3, a SAM, a third dAb and a fourth dAb.

Optionally, the SAM is a tetramerisation domain, eg, a p53 TD.

In an example the first, second, third (when present) and fourth (when present) dAbs have the same antigen binding specificity. In an example the first, second, third (when present) and fourth (when present) dAbs have the same different binding specificity.

In an example the first and second scFvs have the same antigen binding specificity. In an example the first and second scFvs have the same different antigen binding specificity.

In an example the first dAb, second dAb and first scFv have the same antigen binding specificity. In an example the first dAb, second dAb and first scFv have the same different antigen binding specificity.

Herein, where a dAb is provided in the polypeptide, in an alternative there may be provided instead any different type of antigen binding domain, such as a scFv or Fab or non-Ig binding domain (eg, an affibody, avimer or fibronectin domain).

Herein, where a scFv is provided in the polypeptide, in an alternative there may be provided instead any different type of antigen binding domain, such as a dAb or Fab or non-Ig binding domain (eg, an affibody, avimer or fibronectin domain).

Herein, where a Fab is provided in the polypeptide, in an alternative there may be provided instead any different type of antigen binding domain, such as a scFv or dAb or non-Ig binding domain (eg, an affibody, avimer or fibronectin domain).

Each antigen may be any antigen disclosed herein.

In an example, the CH1 (when present), CH2 and CH3 are a human Ig CH1, a CH2 a CH3, eg, a IgG1 CH1, CH2 and CH3. In an example, the CH2 comprises a CH2 domain, the CH3 comprises a CH3 domain and the CH2 comprises a hinge amino acid sequence. In this example, the CH2 comprises (in N- to C-terminal direction) the hinge amino acid sequence and the CH2 domain. In an example the hinge amino acid sequence (i) is a complete hinge; (ii) is a hinge amino acid sequence that is non-functional to dimerise the polypeptide with another such polypeptide; (iii) a hinge amino acid sequence devoid of a hinge core comprising the amino acid motif CXXC (and optionally also devoid of an upper hinge amino acid sequence); or (iv) an upper hinge fused to a lower hinge, but devoid of a hinge core comprising the amino acid motif CXXC; or (v) a lower hinge, but devoid of a hinge core comprising the amino acid motif CXXC (and optionally also devoid of an upper hinge amino acid sequence). Examples of upper, core and lower hinge sequences are disclosed in Table 12. In an example, the CH2 is devoid of a functional hinge region, ie, wherein the hinge region is non-functional to dimerise the polypeptide with another such polypeptide. In an example, the CH2 is devoid of a hinge region. In an example, the CH2 is devoid of a complete hinge region sequence. In an example, the CH2 is devoid of a core hinge region sequence.

In an example, the CH2 comprises (in N- to C-terminal direction) an optional upper hinge region, a lower hinge region and a CH2 domain and wherein the CH2 (and the polypeptide) is devoid of a core hinge region that is functional to dimerise the polypeptide with another said polypeptide.

In an example, the CH2 comprises in N- to C-terminal direction) an optional upper hinge region, a lower hinge region and a CH2 domain and the wherein the CH2 (and the polypeptide) is devoid of a core hinge region amino acid sequence CXXC, wherein X is any amino acid (optionally wherein each amino acid X is selected from a P, R and S).

In an example, the CH2 comprises in N- to C-terminal direction) an amino acid selected from SEQ ID NOs: 163-178 and a CH2 domain and the wherein the CH2 (and the polypeptide) is devoid of a core hinge region amino acid sequence CXXC, wherein X is any amino acid (optionally wherein each amino acid X is selected from a P, R and S).

In an embodiment, the core hinge region amino acid sequence is selected from SEQ ID Nos:

180-182. In an embodiment, the CH2 (an the polypeptide) is devoid of amino acid sequences SEQ ID NOs: 183-187.

In an embodiment, the CH2 domain is a human IgG1 CH2 domain and the core hinge region amino acid sequence is SEQ ID NO: 180. Optionally, any CH1 and CH3 present in the polypeptide are human IgG1 CH1 and CH3 respectively. Optionally, the CH2 (and the polypeptide) is devoid of a core hinge region amino acid sequence CPPC (SEQ ID NO: 180). Optionally, the CH2 (and the polypeptide) is devoid of upper hinge region amino acid sequence EPKSCDKTHT (SEQ ID NO: 183) and core hinge region amino acid sequence CPPC (SEQ ID NO: 180).

In an embodiment, the CH2 domain is a human IgG2 CH2 domain and the core hinge region amino acid sequence is SEQ ID NO: 180. Optionally, any CH1 and CH3 present in the polypeptide are human IgG2 CH1 and CH3 respectively. Optionally, the CH2 (and the polypeptide) is devoid of upper hinge region amino acid sequence ERKCCVE (SEQ ID NO: 184) and core hinge region amino acid sequence CPPC (SEQ ID NO: 180).

In an embodiment, the CH2 domain is a human IgG3 CH2 domain and the core hinge region amino acid sequence is SEQ ID NO: 181. Optionally, any CH1 and CH3 present in the polypeptide are human IgG3 CH1 and CH3 respectively. Optionally, the CH2 (and the polypeptide) is devoid of upper hinge region amino acid sequence ELKTPLGDTIHT (SEQ ID NO: 185) and core hinge region amino acid sequence CPRC (SEQ ID NO: 181). Optionally, alternatively the CH2 (and the polypeptide) is devoid of upper hinge region amino acid sequence EPKSCDTPPP (SEQ ID NO: 186) and core hinge region amino acid sequence CPRC (SEQ ID NO: 181).

In an embodiment, the CH2 domain is a human IgG4 CH2 domain and the core hinge region amino acid sequence is SEQ ID NO: 182. Optionally, any CH1 and CH3 present in the polypeptide are human IgG4 CH1 and CH3 respectively. Optionally, the CH2 (and the polypeptide) is devoid of upper hinge region amino acid sequence ESKYGPP (SEQ ID NO: 187) and core hinge region amino acid sequence CPSC (SEQ ID NO: 182).

In an example, the CH2 of a polypeptide herein is devoid of a core hinge (and optionally also an upper hinge) amino acid sequence. In an example, the CH2 of a polypeptide herein is devoid of a core hinge CXXC amino acid sequence, wherein X is any amino acid, preferably P, R or S, most preferably P. In an example, the CH2 comprises an APELLGGPSV amino acid sequence, or an PAPELLGGPSV amino acid sequence. In an example, the CH2 comprises an APPVAGPSV amino acid sequence, or an PAPPVAGPSV amino acid sequence. In an example, the CH2 comprises an APEFLGGPSV amino acid sequence, or an PAPEFLGGPSV amino acid sequence.

In an example, the CH2 and CH3 of a polypeptide herein are human IgG1 CH2 and CH3 domains, wherein the CH2 is devoid of a core hinge (and optionally also an upper hinge) amino acid sequence, eg, wherein the CH2 is devoid of a CPPC sequence. In an example, the CH2 comprises an APELLGGPSV amino acid sequence, or an EPKSCDKTHT[P]APELLGGPSV amino acid sequence, wherein the bracketed P is optional.

In an example, the CH2 and CH3 of a polypeptide herein are human IgG2 CH2 and CH3 domains, wherein the CH2 is devoid of a core hinge (and optionally also an upper hinge) amino acid sequence, eg, wherein the CH2 is devoid of a CPPC sequence. In an example, the CH2 comprises an APPVAGPSV amino acid sequence, or an ERKCCVE[P]APPVAGPSV amino acid sequence, wherein the bracketed P is optional.

In an example, the CH2 and CH3 of a polypeptide herein are human IgG3 CH2 and CH3 domains, wherein the CH2 is devoid of a core hinge (and optionally also an upper hinge) amino acid sequence, eg, wherein the CH2 is devoid of a CPRC sequence. In an example, the CH2 comprises an APELLGGPSV amino acid sequence, or an ELKTPLGDTTHT[P]APELLGGPSV amino acid sequence, wherein the bracketed P is optional. In an example, the CH2 comprises an EPKSCDTPPP[P]APELLGGPSV amino acid sequence, wherein the bracketed P is optional.

In an example, the CH2 and CH3 of a polypeptide herein are human IgG4 CH2 and CH3 domains, wherein the CH2 is devoid of a core hinge (and optionally also an upper hinge) amino acid sequence, eg, wherein the CH2 is devoid of a CPSC sequence. In an example, the CH2 comprises an APEFLGGPSV amino acid sequence, or an ESKYGPP[P]APEFLGGPSV amino acid sequence, wherein the bracketed P is optional.

When the polypeptide comprises a V-CH1, a CH2 may also be present, but in this case optionally lacking the core hinge region (or at least a sequence selected from CXXC as disclosed herein and SEQ ID Nos: 180-182) and optionally lacking the upper and/or the lower hinge region to prevent F(ab′)2 formation.

Aspects:

By way of example the invention provides the following Aspects, some of which have been exemplified herein.

1. A polypeptide comprising (in N- to C-terminal direction; or in C- to N-terminal direction)
(a) An immunoglobulin superfamily domain;
(b) An optional linker; and
(c) A self-associating multimerisation domain (SAM) (optionally a self-associating tetramerisation domain (TD)).

In an example, each linker is a peptide linker comprising (or comprising up to, or consisting of) 40, 30, 25, 20, 19, 18, 17, 16, 15 14, 13, 12, 11, 10, 9, 8, 7, 6, 5, 4 or amino acids.

In an alternative, the domain of (a) is a non-Ig domain or comprises a non-Ig scaffold.

In an alternative herein, instead of using a TD or copies of a TD, in an embodiment any other self-associating multimerization domain (SAM) may be used. In an example, the SAM (eg, TD) is a human, dog, cat, horse, monkey (eg, cynomolgus monkey), rodent (eg, mouse or rat), rabbit, bird (eg, chicken) or fish SAM (or TD).

Optionally, the domain of (a) is capable of specifically binding to an antigen selected from PD-L1, PD-1, 4-1BB, CTLA-4, 4-1BB, CD28, TNF alpha, IL17 (eg, IL17A), CD38, VEGF-A, EGFR, IL-6, IL-4, IL-6R, IL-4R (eg, IL-4Ra), OX40, OX40L, TIM-3, CD20, GITR, VISTA, ICOS, Death Receptor 5 (DR5), LAG-3, CD40, CD40L, CD27, HVEM, KRAS, haemagglutinin, transferrin receptor 1, amyloid beta, BACE1, Tau, TDP43, SOD1, Alpha Synculein and CD3.

In an example, the antigen is a peptide-MHC.

In some embodiments (eg, some embodiments of Aspect 16 below or 17 below), the polypeptide comprises at least two binding moieties, eg, two dAbs, two scFvs, or a dAb and a scFv. In an example, these binding moieties bind to the same antigen (eg, an antigen disclosed herein or in the immediately preceding paragraph herein). In another example, the moieties bind to different antigens (eg, an antigen disclosed herein or in the immediately preceding paragraph herein). For example, in Aspect 16B, D, E or F the variable domains or scFvs are capable of specifically binding to the same or different antigens selected from TNF alpha, CD38, IL17a, CD20, PD-1, PD-L1, CTLA-4 and 4-1BB. For example, one of the moieties binds to TNF alpha and the other binds to IL17a; one of the moieties binds to PD-1 and the other binds to 4-1BB; or one of the moieties binds to PD-L1 and the other binds to 4-1BB; one of the moieties binds to PD-1 and the other binds to CTLA-4; or one of the moieties binds to PD-L1 and the other binds to CTLA-4.

2. The polypeptide of any preceding Aspect, wherein the domain of (a) is an antibody variable domain.

For example, a variable domain herein is a VH (eg, comprised by a scFv or a Fab polypeptide chain). In another example, it is a VHH (eg, comprised by a scFv or a Fab polypeptide chain). In another example, it is a humanised VH, humanised VHH or a human VH (eg, comprised by a scFv or a Fab polypeptide chain). In another example, it is a VL (eg, comprised by a scFv or a Fab polypeptide chain). In another example, it is a Vκ. In another example, it is a Vλ.

In another example, the domain of (a) is a TCR variable domain (eg, a TCRα, TCRβ, TCRγ or TCRδ).

In an example the immunoglobulin superfamily domain is an antibody single variable domain (dAb).

3. The polypeptide of any preceding Aspect, wherein the domain of (a) is selected from an antibody single variable domain, a VH and a VL; or wherein the domain is comprised by an scFv.

In an example, a single variable domain herein is a human or humanised dAb or nanobody; or is a camelid VHH domain.

In an example, the domain of(a) is comprised by a single-chain TCR (scTCR).

4. The polypeptide of any preceding Aspect, wherein (a) is joined directly to (c); or wherein (b) is joined directly to (a) and (c).
5. The polypeptide of any preceding Aspect, comprising (in N- to C-terminal direction) the SAM, (d) an optional second linker and (e) a second immunoglobulin superfamily domain.
6. The polypeptide of Aspect 5, wherein the second domain is selected from an antibody single variable domain, a VH and a VL; or wherein the domain is comprised by an scFv.

In an example, the single variable domain is a human or humanised dAb or nanobody; or is a camelid VHH domain.

7. The polypeptide of Aspect 5, wherein the second domain is an antibody single variable domain or an antibody constant domain.
8. The polypeptide of Aspect 7, wherein the constant domain is comprised by an antibody Fc region.
9. The polypeptide of any one of Aspects 5 to 8, wherein (c) is joined directly to (e); or wherein (d) is joined directly to (c) and (e).
10. The polypeptide of any preceding Aspect, wherein the TD is a p53, p63 or p73 TD or a homologue or orthologue thereof; or wherein the TD is a NHR2 TD or a homologue or orthologue thereof.

Optionally, the TD herein is a TD of a protein disclosed in Table 2.

11. The polypeptide of any preceding Aspect, wherein the TD comprises an amino acid sequence that is at least 80% identical to SEQ ID NO: 10 or 126.
12. The polypeptide of any preceding Aspect, comprising (f) an antibody variable domain, an antibody constant region or an antibody Fc region between (a) and (c).
13. The polypeptide of Aspect 12, wherein (f) comprises (i) an antibody CH1 constant domain; or (ii) an antibody Fc region (ie, comprising a CH2-CH3).
14. The polypeptide of Aspect 13, wherein the polypeptide is associated with a second polypeptide, wherein (iii) the second polypeptide comprises an antibody CL constant domain that is paired with the CH1 domain of (i); or (iv) the second polypeptide comprises a second antibody Fc region that is paired with the Fc region of (ii).
15. The polypeptide of Aspect 12, wherein the variable domain of (f) is an antibody single variable domain.
16. The polypeptide of any preceding Aspect, wherein the polypeptide (first polypeptide) comprises or consists of (in N- to C-terminal direction);
A. A first antibody single variable domain (dAb), an optional linker and said SAM;
B. A first antibody single variable domain, an optional linker, said SAM and a second antibody single variable domain;
C. A first scFv, an optional linker and said SAM;
D. A first scFv, an optional linker, said SAM and a second scFv;
E. A first antibody single variable domain, an optional linker, said SAM and a first scFv;
F. A first scFv, an optional linker, said SAM and a first antibody single variable domain;
G. A first antibody variable domain, an optional first linker, a first antibody constant domain, a second optional linker and said SAM;
H. Said SAM, an optional linker and a first antibody single variable domain;
I. Said SAM, an optional linker and a first scFv;
J. Said SAM, an optional linker, a first antibody constant domain, a second optional linker and a first antibody variable domain; or
K. Said SAM, an optional linker, a first antibody variable domain, a second optional linker and a first antibody constant domain.

Optionally, each variable domain is a VH or a VL (eg, a Vc or a V).

Optionally, each domain of the polypeptide herein is a human domain. Optionally, each domain of the polypeptide herein is a human or humanised domain.

17. The polypeptide of any of Aspects 1 to 15, wherein
(i) the polypeptide comprises (in N- to C-terminal direction);
A. A dAb and a self-associating multimersiation domain (SAM)
B. A first dAb, a SAM and a second dAb
C. A first scFv and a SAM;
D. A first scFv, a SAM and a second scFv;
E. A first scFv, a SAM and a first dAb;
F. A first dAb, a CH2, a CH3 and a SAM;
G. A first scFv, a CH2, a CH3 and a SAM;

### H. A VH, a CH1, a CH2 a CH3 and a SAM;

### I. A VL, a CL, a CH2, a CH3 and a SAM;

J. A dAb; a SAM, a CH2 and a CH3;
K. A scFv; a SAM, a CH2 and a CH3;

### L. A VH, a CH1, a SAM, a CH2 and a CH3;

### M. A VL, a CL, a SAM, a CH2 and a CH3;

N. A first dAb, a second dAb and a SAM;

### O. A VH, a CH1 and a SAM;

### P. A VL, a CL and a SAM;

Q. A VH, a CH1, a SAM and a first dAb;
R. A VL, a CL, a SAM and a first dAb;
S. A first dAb, a second dAb, a SAM and a third dAb;
T. A first dAb, a second dAb, a SAM and a first scFv;
U. A first dAb, a second dAb, a SAM, a third dAb and a fourth dAb;
V. A first dAb, a CH2, a CH3, a SAM and a second dAb;
W. A first dAb, a CH2, a CH3, a SAM and a first scFv;
X. A first dAb, a second dAb, a CH2, a CH3 and a SAM;
Y. A first dAb, a second dAb, a CH2, a CH3, a SAM and a third dAb;
Z. A first dAb, a second dAb, a CH2, a CH3, a SAM and a first scFv; or
AA. A first dAb, a second dAb, a CH2, a CH3, a SAM, a third dAb and a fourth dAb;

### Or

(ii) the polypeptide comprises (in C- to N-terminal direction);
A. A dAb and a self-associating multimersiation domain (SAM)
B. A first dAb, a SAM and a second dAb
C. A first scFv and a SAM;
D. A first scFv, a SAM and a second scFv;
E. A first scFv, a SAM and a first dAb;
F. A first dAb, a CH2, a CH3 and a SAM;
G. A first scFv, a CH2, a CH3 and a SAM;

### H. A VH, a CH1, a CH2 a CH3 and a SAM;

### I. A VL, a CL, a CH2, a CH3 and a SAM;

J. A dAb; a SAM, a CH2 and a CH3;
K. A scFv; a SAM, a CH2 and a CH3;

### L. A VH, a CH1, a SAM, a CH2 and a CH3;

### M. A VL, a CL, a SAM, a CH2 and a CH3;

N. A first dAb, a second dAb and a SAM;

### O. A VH, a CH1 and a SAM;

### P. A VL, a CL and a SAM;

Q. A VH, a CH1, a SAM and a first dAb;
R. A VL, a CL, a SAM and a first dAb;
S. A first dAb, a second dAb, a SAM and a third dAb;
T. A first dAb, a second dAb, a SAM and a first scFv;
U. A first dAb, a second dAb, a SAM, a third dAb and a fourth dAb;
V. A first dAb, a CH2, a CH3, a SAM and a second dAb;
W. A first dAb, a CH2, a CH3, a SAM and a first scFv;
X. A first dAb, a second dAb, a CH2, a CH3 and a SAM;
Y. A first dAb, a second dAb, a CH2, a CH3, a SAM and a third dAb;
Z. A first dAb, a second dAb, a CH2, a CH3, a SAM and a first scFv; or
AA. A first dAb, a second dAb, a CH2, a CH3, a SAM, a third dAb and a fourth dAb.

Thus, in some embodiments the polypeptide comprises an antibody Fc region, wherein the Fc comprises the CH2 and CH3 domains.

18. The polypeptide of Aspect 16B, 16D, (i) 17B, (i) 17D, (i) 17N, (i) 17S, (i) 17T, (i) 17U, (i) 17X, (i) 17Y, (i) 17Z, (i) 17AA, (ii) 17B, (ii) 17D, (ii) 17N, (ii) 17S, (ii) 17T, (ii) 17U, (ii) 17X, (ii) 17Y, (ii) 17Z or (ii) 17AA wherein the single variable domains (dAbs) are identical; or wherein the scFvs are identical.
19. The polypeptide of Aspect 16B, 16D, (i) 17B, (i) 17D, (i) 17N, (i) 17S, (i) 17T, (i) 17U, (i) 17X, (i) 17Y, (i) 17Z, (i) 17AA, (ii) 17B, (ii) 17D, (ii) 17N, (ii) 17S, (ii) 17T, (ii) 17U, (ii) 17X, (ii) 17Y, (ii) 17Z or (ii) 17AA wherein the single variable domains are different; or wherein the scFvs are different.
20. The polypeptide of Aspect 16G, 16J, 16K, (i) 17H, (i) 171, (i) 17L, (i) 17M, (i) 170, (i) 17P, (i) 17Q, (i) 17R, (ii) 17H, (ii) 171, (ii) 17L, (ii) 17M, (ii) 170, (ii) 17P, (ii) 17Q or (ii) 17R wherein (i) the first variable domain is a VH domain and the first constant domain is a CH1 domain, and optionally the polypeptide is associated with a second polypeptide, wherein the second polypeptide comprises an antibody CL constant domain that is paired with the CH1 domain; (ii) the first variable domain is a VH domain and the first constant domain is a CL domain, and optionally the polypeptide is associated with a second polypeptide, wherein the second polypeptide comprises an antibody CH1 constant domain that is paired with the CL domain; (iii) the first variable domain is a VL domain and the first constant domain is a CH1 domain, and optionally the polypeptide is associated with a second polypeptide, wherein the second polypeptide comprises an antibody CL constant domain that is paired with the CH1 domain; or (iv) the first variable domain is a VL domain and the first constant domain is a CL domain, and optionally the polypeptide is associated with a second polypeptide, wherein the second polypeptide comprises an antibody CH1 constant domain that is paired with the CL domain.
21. The polypeptide of any one of Aspects 16 to 20, comprising an antibody Fc region or a further antibody single variable domain between (v) the first variable domain or scFv and (vi) the SAM, wherein the Fc comprises a CH2 and a CH3.

This further variable domain may be different from the first single variable domain or may have a target binding specificity that is different from the target binding specificity of the first single variable domain or scFv.

22. The polypeptide of any one of Aspects 16 to 21, comprising an antibody Fc region or an antibody single variable domain between (vii) the SAM and (viii) the most C-terminal variable domain, wherein the Fc comprises a CH2 and a CH3.
23. The polypeptide of any one of Aspects 16 to 22, comprising in N- to C-terminal direction an antibody Fc region and the most N-terminal variable domain or scFv, wherein the Fc comprises a CH2 and a CH3.
24. The polypeptide of any one of Aspects 16 to 23, comprising in N- to C-terminal direction the SAM and an antibody Fc region, wherein the Fc comprises a CH2 and a CH3.
25. The polypeptide of any one of Aspects 17 to 24, wherein the CH2 is devoid of an amino acid sequence CXXC or an amino acid sequence selected from SEQ ID NOs: 180-182; and optionally is devoid of amino acid sequences SEQ ID NOs: 183-187.

In an example, the CH2 is a CH2′ disclosed herein. Optionally, the CH2 comprises (in N- to C-terminal direction) an optional upper hinge region, a lower hinge region and a CH2 domain and wherein the CH2 (and the polypeptide) is devoid of a core hinge region that is functional to dimerise the polypeptide with another said polypeptide. Optionally, the CH2 comprises in N- to C-terminal direction) an optional upper hinge region, a lower hinge region and a CH2 domain and the wherein the CH2 (and the polypeptide) is devoid of a core hinge region amino acid sequence CXXC, wherein X is any amino acid (optionally wherein each amino acid X is selected from a P, R and S). Optionally, the CH2 comprises in N- to C-terminal direction) an amino acid selected from SEQ ID NOs: 163-178 and a CH2 domain and the wherein the CH2 (and the polypeptide) is devoid of a core hinge region amino acid sequence CXXC, wherein X is any amino acid (optionally wherein each amino acid X is selected from a P, R and S). In an embodiment, the core hinge region amino acid sequence is selected from SEQ ID Nos: 180-182. In an embodiment, the CH2 (an the polypeptide) is devoid of amino acid sequences SEQ ID NOs: 183-187. In an embodiment, the CH2 domain is a human IgG1 CH2 domain and the core hinge region amino acid sequence is SEQ ID NO: 180. Optionally, any CH1 and CH3 present in the polypeptide are human IgG1 CH1 and CH3 respectively. Optionally, the CH2 (and the polypeptide) is devoid of a core hinge region amino acid sequence CPPC (SEQ ID NO: 180). Optionally, the CH2 (and the polypeptide) is devoid of upper hinge region amino acid sequence EPKSCDKTHT (SEQ ID NO: 183) and core hinge region amino acid sequence CPPC (SEQ ID NO: 180). In an embodiment, the CH2 domain is a human IgG2 CH2 domain and the core hinge region amino acid sequence is SEQ ID NO: 180. Optionally, any CH1 and CH3 present in the polypeptide are human IgG2 CH1 and CH3 respectively. Optionally, the CH2 (and the polypeptide) is devoid of upper hinge region amino acid sequence ERKCCVE (SEQ ID NO: 184) and core hinge region amino acid sequence CPPC (SEQ ID NO: 180). In an embodiment, the CH2 domain is a human IgG3 CH2 domain and the core hinge region amino acid sequence is SEQ ID NO: 181. Optionally, any CH1 and CH3 present in the polypeptide are human IgG3 CH1 and CH3 respectively. Optionally, the CH2 (and the polypeptide) is devoid of upper hinge region amino acid sequence ELKTPLGDTIHT (SEQ ID NO: 185) and core hinge region amino acid sequence CPRC (SEQ ID NO: 181). Optionally, alternatively the CH2 (and the polypeptide) is devoid of upper hinge region amino acid sequence EPKSCDTPPP (SEQ ID NO: 186) and core hinge region amino acid sequence CPRC (SEQ ID NO: 181). In an embodiment, the CH2 domain is a human IgG4 CH2 domain and the core hinge region amino acid sequence is SEQ ID NO: 182. Optionally, any CH1 and CH3 present in the polypeptide are human IgG4 CH1 and CH3 respectively. Optionally, the CH2 (and the polypeptide) is devoid of upper hinge region amino acid sequence ESKYGPP (SEQ ID NO: 187) and core hinge region amino acid sequence CPSC (SEQ ID NO: 182).

26. The polypeptide of any preceding Aspect, wherein the first or each linker is a (G₄S)ₙ linker, wherein n=1, 2, 3, 4, 5, 6, 7, 8, 9 or 10.

In an example, n is 3. In an example, n is 4. In an example, n is 5.

27. The polypeptide of any preceding Aspect, wherein each domain and SAM is a human domain and SAM respectively.
28. The polypeptide of any preceding Aspect, wherein each variable domain or scFv is capable of binding to an antigen.

In an example, the binding antagonises the antigen. In another example, the binding agonises the antigen.

29. The polypeptide of any preceding Aspect, wherein the polypeptide comprises binding specificity for more than one antigen, optionally 2, 3 or 4 different antigens.

For example, the polypeptide comprises at least one anti-CTLA-4 binding domain (eg, dAb or scFv) and at least one anti-4-1BB binding domain. For example, the polypeptide comprises at least one anti-CTLA-4 binding domain (eg, dAb or scFv) and at least one anti-PD-L1 binding domain. For example, the polypeptide comprises at least one anti-CTLA-4 binding domain (eg, dAb or scFv) and at least one anti-PD-1 binding domain. For example, the polypeptide comprises at least one anti-TNF alpha binding domain (eg, dAb or scFv) and at least one anti-IL-17A binding domain.

For example, the polypeptide comprises a first antigen binding domain (eg, a said VH, VL, VHH, dAb, scFv or Fab variable region) that is N-terminal of the SAM and a second antigen binding domain (eg, a said VH, VL, VHH, dAb, scFv or Fab variable region) that is C-terminal of the SAM. In an example, the polypeptide comprises a third antigen binding domain (eg, a said VH, VL, VHH, dAb, scFv or Fab variable region) that is N-terminal of the SAM (eg, and also N-terminal of the first domain; or between the first domain and the SAM); and optionally the polypeptide a fourth antigen binding domain (eg, a said VH, VL, VHH, dAb, scFv or Fab variable region) that is C-terminal of the SAM (eg, and also C-terminal of the second domain; or between the second domain and the SAM).

In an embodiment, the first domain is capable of specifically binding to an immune checkpoint or T-cell co-stimulatory antigen (eg, selected from OX40, GITR, VISTA, CD40, CD28, LAG3 and TIM-3) and the second binding site is capable of specifically binding to an immune checkpoint or T-cell co-stimulatory antigen (eg, selected from OX40, GITR, VISTA, CD40, CD28, LAG3 and TIM-3). In an example, the domains have the same antigen binding specificity. In an example, the domains have the same epitope binding specificity. In an example, the domains have different antigen binding specificity. In an example, the domains have different epitope binding specificity on the same antigen. In an example, the domains bind TNF alpha. In an example, the domains bind CD20. In an example, the domains bind PD-1. In an example, the domains bind PD-L1. In an example, the domains bind CTLA-4.

In an embodiment, the first domain is capable of specifically binding to 4-1BB, PD-1 or PD-L1 and the second binding site is capable of specifically binding to CTLA-4. In an embodiment, the second domain is capable of specifically binding to 4-1BB, PD-1 or PD-L1 and the first binding site is capable of specifically binding to CTLA-4. In an example, the first domain is capable of specifically binding to 4-1BB, the second binding site is capable of specifically binding to CTLA-4, the third binding site is capable of specifically binding to an immune checkpoint or T-cell co-stimulatory antigen (eg, selected from OX40, GITR, VISTA, CD40, CD28, LAG3 and TIM-3) and the fourth binding site is capable of specifically binding to an immune checkpoint or T-cell co-stimulatory antigen (eg, selected from OX40, GITR, VISTA, CD40, CD28, LAG3 and TIM-3). In an example, the first domain is capable of specifically binding to PD-1, the second binding site is capable of specifically binding to CTLA-4, the third binding site is capable of specifically binding to an immune checkpoint or T-cell co-stimulatory antigen (eg, selected from OX40, GITR, VISTA, CD40, CD28, LAG3 and TIM-3) and the fourth binding site is capable of specifically binding to an immune checkpoint or T-cell co-stimulatory antigen (eg, selected from OX40, GITR, VISTA, CD40, CD28, LAG3 and TIM-3). In an example, the first domain is capable of specifically binding to PD-L1, the second binding site is capable of specifically binding to CTLA-4, the third binding site is capable of specifically binding to CTLA-4, PD-L1, CD3 or CD28 and the fourth binding site is capable of specifically binding to an immune checkpoint or T-cell co-stimulatory antigen (eg, selected from OX40, GITR, VISTA, CD40, CD28, LAG3 and TIM-3).

In an embodiment, the first domain is capable of specifically binding to TNF alpha and the second binding site is capable of specifically binding to IL-17 (eg, IL-17A). In an embodiment, the second domain is capable of specifically binding to TNF alpha and the first binding site is capable of specifically binding to IL-17 (eg, IL-17A).

In an example, the polypeptide comprises a cytokine, eg, an IL-2, IL-15 or IL-21. In an example, the cytokine is a truncated cytokine, eg, a truncated IL-2, IL-15 or IL-21. In an example, the cytokine is C-terminal of the SAM (eg, C-terminal of the C-terminal most antigen binding domain). In an example, the cytokine is N-terminal of the SAM (eg, N-terminal of the N-terminal most antigen binding domain). In an embodiment of these examples, the first domain is capable of specifically binding to an immune checkpoint or T-cell co-stimulatory antigen (eg, selected from OX40, GITR, VISTA, CD40, CD28, LAG3 and TIM-3). In an embodiment of these examples, the first domain is capable of specifically binding to 4-1BB, PD-1, PD-L1 or CTLA-4. In an embodiment of these examples, the second domain is capable of specifically binding to an immune checkpoint or T-cell co-stimulatory antigen (eg, selected from OX40, GITR, VISTA, CD40, CD28, LAG3 and TIM-3). In an embodiment of these examples, the second domain is capable of specifically binding to 4-1BB, PD-1, PD-L1 or CTLA-4. In an embodiment of these examples, the third domain is capable of specifically binding to an immune checkpoint or T-cell co-stimulatory antigen (eg, selected from OX40, GITR, VISTA, CD40, CD28, LAG3 and TIM-3). In an embodiment of these examples, the third domain is capable of specifically binding to 4-1BB, PD-1, PD-L1 or CTLA-4. In an embodiment of these examples, the fourth domain is capable of specifically binding to an immune checkpoint or T-cell co-stimulatory antigen (eg, selected from OX40, GITR, VISTA, CD40, CD28, LAG3 and TIM-3). In an embodiment of these examples, the fourth domain is capable of specifically binding to 4-1BB, PD-1, PD-L1 or CTLA-4.

30. A multimer (optionally a tetramer) of a polypeptide according to any preceding Aspect.

In an example, the multimer is a polypeptide dimer.

In an example, the multimer is a polypeptide trimer.

In an example, the multimer is a polypeptide tetramer.

31. The polypeptide or multimer of any preceding Aspect, comprising eukaryotic cell glycosylation.
32. The polypeptide or multimer of Aspect 31, wherein the cell is a HEK293, CHO or Cos cell.
33. The polypeptide or multimer of any preceding Aspect for medical use.
34. A pharmaceutical composition comprising the polypeptide or multimer of any preceding Aspect.
35. A nucleic acid encoding a polypeptide of any one of Aspects 1 to 29 and 31 to 33.
36. A eukaryotic cell or vector comprising the nucleic acid of Aspect 35.
37. A method of binding multiple copies of an antigen, the method comprising combining the copies with a multimer of any one of Aspects 30 to 33, wherein the copies are bound by polypeptides of the multimer, and optionally the method comprising isolating the multimer bound to the antigen copies.
38. The method of Aspect 37 wherein the method is a diagnostic method for detecting the presence of a substance in a sample, wherein the substance comprises the antigen, the method comprising providing the sample (eg, a bodily fluid, food, food ingredient, beverage, beverage ingredient, soil or forensic sample), mixing the sample with multimers according to any one of Aspects 30 to 33 and detecting the binding of multimers to the antigen in the sample.

In an example the bodily fluid is a blood, saliva, semen or urine sample.

In an example, the method is for pregnancy testing or diagnosing a disease or condition in a subject from which the sample has been previously obtained.

39. A method of treating or reducing the risk of a disease or condition in a human or animal subject, the method comprising administering the composition of Aspect 34 to the subject, wherein multimers comprised by the composition specifically bind to a target antigen in the subject, wherein said binding mediates the treatment or reduction in risk.
40. The method of Aspect 39, wherein the antigen is an immune checkpoint or T-cell co-stimulatory antigen (eg, PD-L1, PD-1 or CTLA4); or wherein the antigen is TNF alpha or IL-17A.
41. The method of Aspect 39, wherein the antigen mediates the disease or condition in the subject; and optionally wherein the binding antagonises the antigen.

In another embodiment, the binding agonises the antigen.

42. A composition comprising a plurality of polypeptides according to any one of Aspects 1 to 29 and 31 to 33, wherein at least 90% of the polypeptides are comprised by tetramers of said polypeptides.

Optionally, at least 91, 92, 93, 94, 95, 96, 97, 98 or 99/a of the polypeptides are comprised by tetramers of said polypeptides.

43. The composition of Aspect 42, wherein at least 98% of the polypeptides are comprised by tetramers of said polypeptides.
44. The composition of Aspect 42 or 43, wherein the remaining (ie, the balance to 100% of polypeptide) polypeptides are selected from one or more of polypeptide monomers, dimers and trimers.
45. A method of producing a composition (optionally a composition according to any one of Aspects 42 to 44) comprising a plurality of polypeptides according to any one of Aspects 1 to 29 and 31 to 33, the method comprising providing eukaryotic host cells according to Aspect 34, culturing the host cells, and allowing expression and secretion from the cells of tetramers of the polypeptides, and optionally isolating or purifying the tetramers.

Any of these Aspects is combinable with any other disclosure herein, eg, any of the Clauses or Paragraphs.

### Polypeptides & Multimers Comprising FC

The invention also provides polypeptides and multimers comprising antibody Fc region(s). This is useful, for example, to harness FcRn recycling when administered to a subject, such as a human or animal, which may contribute to a desirable half-life in vivo. Fc regions are also useful for providing Fc effector functions. For example, an IgG1 Fc may be useful when the multimer is used to treat a cancer or where cell killing is desired, eg, by ADCC.

To this end, the invention provides the following:

A polypeptide comprising an antibody Fc region, wherein the Fc region comprises an antibody CH2 and an antibody CH3; and a self-associating multimerisation domain (SAM); wherein the CH2 comprises an antibody hinge sequence and is devoid of a core hinge region.

In an embodiment, the polypeptide comprises an epitope binding site, eg, an antibody VH single variable domain or an antibody VH/VL pair that binds to an epitope. Additionally or alternatively, the polypeptide comprises an epitope which is cognate to an antibody. This is useful, for example, as the polypeptide can form a multimer that binds copies of the antibodies, such as when the multimer is contacted with a sample comprising the antibodies (eg, for medical use as disclosed herein). In this way, for example, the multimer can be used in a method of diagnosis or testing to determine the presence and/or quantity (or relative amount) of the antibody in the sample. As the multimer provides multiple copies of the epitope (at least one for each polypeptide comprised by the multimer), this can be useful to bind many copies of the antibody, which may be present in relatively small amounts in the sample, thereby having the effect of enhancing the chances of detecting (or amplifying) a positive signal denoting presence of the antibody. Thus, assay sensitivity may be enhanced so that relatively rare antibodies in samples can be detected.

Optionally, the CH2 is devoid of (i) a core hinge CXXC amino acid sequence, wherein X is any amino acid or wherein each amino acid X is selected from a P, R and S; and/or (ii) an upper hinge amino acid sequence. For the CH2 is devoid of (i) a core hinge CXXC amino acid sequence, wherein X is any amino acid or wherein each amino acid X is selected from a P, R and S and the Fc does not directly pair with another Fc.

Optionally, the CXXC sequence is selected from SEQ ID NOs: 180-182; or the CH2 is devoid of amino acid sequences SEQ ID NOs: 183-187.

Optionally, the CH2 comprises

A. amino acid sequence
(SEQ ID NO: 163)
APELLGGPSV,
or
(SEQ ID NO: 164)
PAPELLGGPSV;
B. amino acid sequence
(SEQ ID NO: 165)
APPVAGPSV,
or
(SEQ ID NO: 166)
PAPPVAGPSV;
C. amino acid sequence
(SEQ ID NO: 175)
APEFLGGPSV,
or
(SEQ ID NO: 176)
PAPEFLGGPSV;
D. amino acid sequence
(SEQ ID NO: 167 or 168)
EPKSCDKTHT[P]APELLGGPSV,
wherein the bracketed P is optional;
E. amino acid sequence
(SEQ ID NO: 169 or 170)
ERKCCVE[P]APPVAGPSV,
wherein the bracketed P is optional;
F. amino acid sequence
(SEQ ID NO: 171 or 172)
ELKTPLGDTTHT[P]APELLGGPSV,
wherein the bracketed P is optional;
G. amino acid sequence
(SEQ ID NO: 173 or 174)
EPKSCDTPPP[P]APELLGGPSV,
wherein the bracketed P is optional;
or
H. amino acid sequence
(SEQ ID NO: 177 or 178)
ESKYGPP[P]APEFLGGPSV,
wherein the bracketed P is optional.

Optionally, the CH2 comprises (in N- to C-terminal direction) an optional upper hinge region, a lower hinge region and a CH2 domain and wherein the CH2 (and the polypeptide) is devoid of a core hinge region that is functional to dimerise the polypeptide with another said polypeptide. Optionally, the CH2 comprises in N- to C-terminal direction) an optional upper hinge region, a lower hinge region and a CH2 domain and the wherein the CH2 (and the polypeptide) is devoid of a core hinge region amino acid sequence CXXC, wherein X is any amino acid (optionally wherein each amino acid X is selected from a P, R and S). Optionally, the CH2 comprises in N- to C-terminal direction) an amino acid selected from SEQ ID NOs: 163-178 and a CH2 domain and the wherein the CH2 (and the polypeptide) is devoid of a core hinge region amino acid sequence CXXC, wherein X is any amino acid (optionally wherein each amino acid X is selected from a P, R and S). In an embodiment, the core hinge region amino acid sequence is selected from SEQ ID Nos: 180-182. In an embodiment, the CH2 (an the polypeptide) is devoid of amino acid sequences SEQ ID NOs: 183-187. In an embodiment, the CH2 domain is a human IgG1 CH2 domain and the core hinge region amino acid sequence is SEQ ID NO: 180. Optionally, any CH1 and CH3 present in the polypeptide are human IgG1 CH1 and CH3 respectively. Optionally, the CH2 (and the polypeptide) is devoid of a core hinge region amino acid sequence CPPC (SEQ ID NO: 180). Optionally, the CH2 (and the polypeptide) is devoid of upper hinge region amino acid sequence EPKSCDKTHT (SEQ ID NO: 183) and core hinge region amino acid sequence CPPC (SEQ ID NO: 180). In an embodiment, the CH2 domain is a human IgG2 CH2 domain and the core hinge region amino acid sequence is SEQ ID NO: 180. Optionally, any CH1 and CH3 present in the polypeptide are human IgG2 CH1 and CH3 respectively. Optionally, the CH2 (and the polypeptide) is devoid of upper hinge region amino acid sequence ERKCCVE (SEQ ID NO: 184) and core hinge region amino acid sequence CPPC (SEQ ID NO: 180). In an embodiment, the CH2 domain is a human IgG3 CH2 domain and the core hinge region amino acid sequence is SEQ ID NO: 181. Optionally, any CH1 and CH3 present in the polypeptide are human IgG3 CH1 and CH3 respectively. Optionally, the CH2 (and the polypeptide) is devoid of upper hinge region amino acid sequence ELKTPLGDTIHT (SEQ ID NO: 185) and core hinge region amino acid sequence CPRC (SEQ ID NO: 181). Optionally, alternatively the CH2 (and the polypeptide) is devoid of upper hinge region amino acid sequence EPKSCDTPPP (SEQ ID NO: 186) and core hinge region amino acid sequence CPRC (SEQ ID NO: 181). In an embodiment, the CH2 domain is a human IgG4 CH2 domain and the core hinge region amino acid sequence is SEQ ID NO: 182. Optionally, any CH1 and CH3 present in the polypeptide are human IgG4 CH1 and CH3 respectively. Optionally, the CH2 (and the polypeptide) is devoid of upper hinge region amino acid sequence ESKYGPP (SEQ ID NO: 187) and core hinge region amino acid sequence CPSC (SEQ ID NO: 182).

Optionally, the polypeptide comprises an antibody CH1-hinge sequence devoid of core region-CH2-CH3.

Optionally, the CH2 and CH3 comprise

A. human IgG1 CH2 and CH3 domains;

B. human IgG2 CH2 and CH3 domains;

C. human IgG3 CH2 and CH3 domains; or

D. human IgG4 CH2 and CH3 domains.

Optionally, the CH2 and CH3 comprise
(a) human IgG1 CH2 and CH3 domains and the hinge sequence and core hinge region is a human IgG1 hinge sequence and hinge region;
(b) human IgG2 CH2 and CH3 domains and the hinge sequence and core hinge region is a human IgG2 hinge sequence and hinge region;
(c) human IgG3 CH2 and CH3 domains and the hinge sequence and core hinge region is a human IgG31 hinge sequence and hinge region; or
(d) human IgG4 CH2 and CH3 domains and the hinge sequence and core hinge region is a human IgG4 hinge sequence and hinge region

Optionally,
A. the CH2 domain comprises a human IgG1 CH2 domain and the core hinge region amino acid sequence is SEQ ID NO: 180, optionally wherein the CH2 is devoid of upper hinge region amino acid sequence EPKSCDKTHT (SEQ ID NO: 183);
B. the CH2 domain comprises a human IgG2 CH2 domain and the core hinge region amino acid sequence is SEQ ID NO: 180, optionally wherein the CH2 is devoid of upper hinge region amino acid sequence ERKCCVE (SEQ ID NO: 184);
C. the CH2 domain comprises a human IgG3 CH2 domain and the core hinge region amino acid sequence is SEQ ID NO: 181, optionally wherein the CH2 is devoid of upper hinge region amino acid sequence ELKTPLGDTTHT (SEQ ID NO: 185) or upper hinge region amino acid sequence EPKSCDTPPP (SEQ ID NO: 186); or
D. the CH2 domain comprises a human IgG4 CH2 domain and the core hinge region amino acid sequence is SEQ ID NO: 182, and optionally wherein the CH2 is devoid of upper hinge region amino acid sequence ESKYGPP (SEQ ID NO: 187).

Optionally, the polypeptide comprises (in N- to C-terminal direction) the Fc region and the SAM, the Fc region comprising (in N- to C-terminal direction) the hinge sequence, a CH2 domain and a CH3 domain.

Optionally, the polypeptide comprises one or more epitope binding sites, eg, an antibody variable domain that is capable of specifically binding to a first epitope. Optionally, the first epitope is comprised by an antigen (eg, a human antigen) selected from the group consisting of ABCF1; ACVR1; ACVR1B; ACVR2; ACVR2B; ACVRL1; ADORA2A; Aggrecan; AGR2; AICDA; AWI; AIG1; AKAP1; AKAP2; AIYIH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BM PI; BMP2; BMP3B (GDFIO); BMP4; BMP6; BM P8; BMPRIA; BMPRIB; BM PR2; BPAG1 (plectin); BRCA1; CI9orflO (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-id); CCL16 (HCC-4); CCL17 (TARC); CCL18 (PARC); CCL19 (M IP-3b); CCL2 (MCP-1); MCAF; CCL20 (MIP-3a); CCL21 (MIP-2); SLC; exodus-2; CCL22 (MDC/STC-1); CCL23 (M PIF-1); CCL24 (MPIF-2 I eotaxin-2); CCL25 (TECK); CCL26 (eotaxin-3); CCL27 (CTACK/ILC); CCL28; CCL3 (MIP-la); CCL4 (M IP-lb); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CM KBR5/ChemR13); CCR6 (CMKBR6/CKR-L3/STRL22/DRY6); CCR7 (CKR7/EBI1); CCR8 (CM KBR8/TERI/CKR-L1); CCR9 (GPR-9-6); CCRL1 (VSHK1); CCRL2 (L-CCR); CD164; CD19; CD 1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p2IWapl/Cipl); CDKN1B (p27Kipl); CDKNIC; CDKN2A (pl6INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYDi); CX3CR1 (V28); CXCL1 (GRO1); CXCLIO (IP-10); CXCL11 (1-TAC/IP-9); CXCL12 (SDF1); CXCL13; CXCL14; CXCL16; CXCL2 (GR02); CXCL3 (GR03); CXCL5 (ENA-78 I LIX); CXCL6 (GCP-2); CXCL9 (MIG); CXCR3 (GPR9/CKR-L2); CXCR4; CXCR6 (TYMSTR ISTRL33 I Bonzo); CYB5; CYC1; CYSLTR1; DAB2IP; DES; DKFZp451J0118; DNCL1; DPP4; E2F1; ECGF1; EDG1; EFNAI; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FU12584; FU25530; FLRT1 (fibronectin); FLT1; FOS; FOSL1 (FRA-I); FY (DARC); GABRP (GABAa); GAGEB1; GAGEC1; GALNAC4S-65T; GATA3; GDF5; GFI1; GGT1; GM-CSF; GNAS1; GNRH1; GPR2 (CCRIO); GPR31; GPR44; GPR81 (FKSG80); GRCCIO (CIO); GRP; GSN (Gelsolin); GSTP1; HAVCR2; HDAC4; EDAC5; HDAC7A; HDAC9; HGF; HIF1A; HIP1; histamine and histamine receptors; HLA-A; HLA-DRA; HM74; HMOX1; HUMCYT2A; ICEBERG; ICOSL; 1D2; IFN-a; IFNA1; IFNA2; IFNA4; IFNA5; IFNA6; IFNA7; IFNB1; IFNgamma; TFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; 1L18; IL18BP; IL18R1; IL18RAP; IL19; ILIA; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2 IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; TL7R; IL8; IL8RA; IL8RB; IL8RB; IL9; IL9R; ILK; INHA; INHBA; INSL3; INSL4; IRAKI; IRAK2; ITGA1; ITGA2; 1TGA3; ITGA6 (a6 integrin); ITGAV; ITGB3; ITGB4 (b 4 integrin); JAG1; JAK1; JAK3; JUN; K6HF; KAI1; KDR; MTLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; M IB1; midkine; M IF; M IP-2; MK167 (Ki-67); MMP2; M MP9; MS4A1; MSMB; MT3 (metallothionectin-ifi); MTSS 1; M UC 1 (mucin); MYC; MYD88; NCK2; neurocan; NFKB 1; NFKB2; NGFB (NGF); NGFR; NgR-Lingo; NgR-Nogo66 (Nogo); NgR-p75; NgR-Troy; NM E1 (NM23A); NOX5; NPPB; NROB1; NROB2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PARTI; PATE; PAWR; PCA3; PCNA; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p2IRac2); RARB; RGS1; RGS13; RGS3; RNF110 (ZNF144); ROB02; S100A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINIA3; SERPINB5 (maspin); SERPINE1 (PAT-i); SERPINF1; SHBG; SLA2; SLC2A2; SLC33AI; SLC43AI; SLIT2; SPP1; SPRRIB (Spri); ST6GAL1; STAB1; STAT6; STEAP; STEAP2; TB4R2; TBX21; TCPIO; TDGF1; TEK; TGFA; TGFB1; TGFB1I1; TGFB2; TGFB3; TGFBI; TGFBR1; TGFBR2; TGFBR3; TH1L; THBS1(thrombospondin-1); THBS2; THBS4; THPO; TIE (Tie-i); T]MP3; tissue factor; TLRIO; TLR2; TLR3; TLR4; TLR5; TLR6; TLR7; TLR8; TLR9; TNF; TNF-a; TNFAIP2 (B94); TNFAIP3; TNFRSF1 1A; TNFRSF1A; TNFRSF1B; TNFRSF21; TNFRSF5; TNFRSF6 (Fas); TNFRSF7; TNFRSF8; TNFRSF9; TNFSFIO (TRAIL); TNFSF1 1 (TRANCE); TNFSF12 (AP03L); TNFSF13 (April); TNFSF13B; TNFSF14 (HVEM-L); TNFSF1 5 (VEGI); TNFSF1 8; TNFSF4 (0X40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase lia); TP53; TPM 1; TPM2; TRADD; TRAF1; TRAF2; TRAF3; TRAF4; TRAF5; TRAF6; TREM 1; TREM2; TRPC6; TSLP; TWEAK; VEGF; VEGFB; VEGFC; versican; VHL C5; VLA-4; XCL1 (lymphotactin); XCL2 (SCM-lb); XCR1 (GPR5/CCXCR1); YY1; and ZFPM2. Optionally, the second epitope (as discussed below) is comprised by the same antigen as the first epitope (eg, comprised by the same antigen molecule). In another example, the second antigen is comprised by said group. In an example, the first and second epitopes are comprised by different antigens selected from said group.

For example, the polypeptide has 1, 2, 3, 4 or 5 epitope binding sites (optionally wherein the polypeptide comprises 2 or more binding sites (eg, single variable domains) that bind to different epitopes, or wherein the polypeptide binding sites are identical). In an embodiment, the SAM is a TD (eg, a p53 TD, such as a human p53 TD) and the polypeptide has 2, 3 or 4 binding sites, such as 3 sites or such as 4 sites. Preferably, the polypeptide has 3 binding sites. Preferably, the polypeptide has 4 binding sites. For example, the binding sites each binds TNF alpha (eg, wherein the binding sites are identical, eg, identical antibody single variable domains).

In an example, the multimer is an octavalent bispecific multimer comprising 4 copies of an anti-PD-L1 binding site (eg, dAb) and 4 copies of an anti-4-1BB binding site (eg, dAb).

In an example, the multimer is a tetravalent multimer comprising copies of an anti-PD-L1 binding site (eg, dAb). In an example, the multimer is an octavalent multimer comprising copies of an anti-PD-L1 binding site (eg, dAb). In an example, the multimer is a 12-valent multimer comprising copies of an anti-PD-L1 binding site (eg, dAb). In an example, the multimer is a 16-valent multimer comprising copies of an anti-PD-L1 binding site (eg, dAb). In an example, the anti-PD-L1 binding site comprises an avelumab or atezolizumab binding site that specifically binds to PD-L1. Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-PD-1 binding site (eg, dAb). In an example, the multimer is an octavalent multimer comprising copies of an anti-PD-1 binding site (eg, dAb). In an example, the multimer is a 12-valent multimer comprising copies of an anti-PD-1 binding site (eg, dAb). In an example, the multimer is a 16-valent multimer comprising copies of an anti-PD-1 binding site (eg, dAb). In an example, the anti-PD-1 binding site comprises a nivolumab or pembrolizumab binding site that specifically binds to PD-1. Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-DR5 (Death Receptor 5) binding site (eg, dAb). In an example, the multimer is an octavalent multimer comprising copies of an anti-DR5 binding site (eg, dAb). In an example, the multimer is a 12-valent multimer comprising copies of an anti-DR5 binding site (eg, dAb). In an example, the multimer is a 16-valent multimer comprising copies of an anti-DR5 binding site (eg, dAb). Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-OX40 or OX40L binding site (eg, dAb). In an example, the multimer is an octavalent multimer comprising copies of an anti-OX40 or OX40L binding site (eg, dAb). In an example, the multimer is a 12-valent multimer comprising copies of an anti-OX40 or OX40L binding site (eg, dAb). In an example, the multimer is a 16-valent multimer comprising copies of an anti-OX40 or OX40L binding site (eg, dAb). Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-glucocorticoid-induced tumor necrosis factor receptor (GITR) binding site (eg, dAb). In an example, the multimer is an octavalent multimer comprising copies of an anti-GITR binding site (eg, dAb). In an example, the multimer is a 12-valent multimer comprising copies of an anti-GITR binding site (eg, dAb). In an example, the multimer is a 16-valent multimer comprising copies of an anti-GITR binding site (eg, dAb). Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-antibody kappa light chain (KLC) binding site (eg, dAb). In an example, the multimer is an octavalent multimer comprising copies of an anti-KLC binding site (eg, dAb). In an example, the multimer is a 12-valent multimer comprising copies of an anti-KLC binding site (eg, dAb). In an example, the multimer is a 16-valent multimer comprising copies of an anti-KLC binding site (eg, dAb). Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-VEGF binding site (eg, dAb). In an example, the multimer is an octavalent multimer comprising copies of an anti-VEGF binding site (eg, dAb). In an example, the multimer is a 12-valent multimer comprising copies of an anti-VEGF binding site (eg, dAb). In an example, the multimer is a 16-valent multimer comprising copies of an anti-VEGF binding site (eg, dAb). In an example, the anti-VEGF binding site comprises a VEGF receptor domain that specifically binds to VEGF (eg, a VEGF binding site of human flt (eg, flt-1) or KDR, eg, Ig domain 2 from VEGFR1 or Ig domain 3 from VEGFR2)). In an example, the anti-VEGF binding site comprises an aflibercept, bevacizumab or ranibizumab binding site that specifically binds to VEGF. Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

In an example, the multimer is a tetravalent multimer comprising copies of an anti-TNF alpha binding site (eg, dAb). In an example, the multimer is a tetravalent multimer comprising copies of an anti-TNF alpha binding site (eg, dAb). In an example, the multimer is an octavalent multimer comprising copies of an anti-TNF alpha binding site (eg, dAb). In an example, the multimer is a 12-valent multimer comprising copies of an anti-TNF alpha binding site (eg, dAb). In an example, the multimer is a 16-valent multimer comprising copies of an anti-TNF alpha binding site (eg, dAb). Preferably, in these examples the SAM domain is a TD, eg, a p53 TD, such as a human p53 TD. For the tetramer, each polypeptide comprises one copy of the binding site. For the octamer each polypeptide comprises 2 copies of the binding site. For the 12-mer each polypeptide comprises 3 copies of the binding site. For the 16-mer each polypeptide comprises 4 copies of the binding site.

Optionally, the variable domain is selected from an antibody single variable domain, a VH and a VL; or wherein the domain is comprised by an scFv. Optionally, the domain is comprised by an antibody VH/VL pair that binds to said first epitope. In an example, epitope binding herein is specific binding as herein defined.

Optionally, the polypeptide comprises (in N- to C-terminal direction)
A. the variable domain, the SAM and the Fc region;
B. the Fc region, the SAM and the variable domain;
C. the variable domain, the Fc region and the SAM;
D. the SAM, the variable domain and the Fc region; or
E. the SAM, the Fc region and the variable domain.

Optionally, the polypeptide comprises a second antibody variable domain N- or C-terminal to the SAM, wherein the second variable domain is capable of specifically binding to a second epitope, wherein the first and second epitopes are identical or different.

Optionally, the SAM is a self-associating tetramerisation domain (TD); optionally wherein the TD is a p53, p63 or p73 TD or a homologue or orthologue thereof; or wherein the TD is a NHR2 TD or a homologue or orthologue thereof; or wherein the TD comprises an amino acid sequence that is at least 80, 85, 90, 91, 92, 93, 94, 95, 96, 97, 98 or 99% identical to SEQ ID NO: 10 or 126.

Optionally,

(i) the polypeptide comprises (in N- to C-terminal direction);
A. A first antibody single variable domain (dAb), an optional linker and said SAM;
B. A first antibody single variable domain, an optional linker, said SAM and a second antibody single variable domain;
C. A first scFv, an optional linker and said SAM;
D. A first scFv, an optional linker, said SAM and a second scFv;
E. A first antibody single variable domain, an optional linker, said SAM and a first scFv;
F. A first scFv, an optional linker, said SAM and a first antibody single variable domain;
G. A first antibody variable domain, an optional first linker, a first antibody constant domain, a second optional linker and said SAM;
H. Said SAM, an optional linker and a first antibody single variable domain;
I. Said SAM, an optional linker and a first scFv;
J. Said SAM, an optional linker, a first antibody constant domain, a second optional linker and a first antibody variable domain; or
K. Said SAM, an optional linker, a first antibody variable domain, a second optional linker and a first antibody constant domain;

### Or

(ii) the polypeptide comprises (in N- to C-terminal direction);
A. A dAb and the SAM;
B. A first dAb, the SAM and a second dAb;
C. A first scFv and the SAM;
D. A first scFv, the SAM and a second scFv;
E. A first scFv, the SAM and a first dAb;
F. A first dAb, the Fc region and the SAM;
G. A first scFv, the Fc region and the SAM;
H. A VH, a CH1, the Fc region and the SAM;
I. A VL, a CL, the Fc region and the SAM;
J. A dAb; the SAM and the Fc region;
K. A scFv; the SAM and the Fc region;
L. A VH, a CH1, the SAM and the Fc region;
M. A VL, a CL, the SAM and the Fc region;
N. A first dAb, a second dAb and the SAM;
O. A VH, a CH1 and the SAM;
P. A VL, a CL and the SAM;
Q. A VH, a CH1, the SAM and a first dAb;
R. A VL, a CL, the SAM and a first dAb;
S. A first dAb, a second dAb, the SAM and a third dAb;
T. A first dAb, a second dAb, the SAM and a first scFv;
U. A first dAb, a second dAb, the SAM, a third dAb and a fourth dAb;
V. A first dAb, the Fc region, the SAM and a second dAb;
W. A first dAb, the Fc region, the SAM and a first scFv;
X. A first dAb, a second dAb, the Fc region and the SAM;
Y. A first dAb, a second dAb, the Fc region, the SAM and a third dAb;
Z. A first dAb, a second dAb, the Fc region, the SAM and a first scFv; or
AA. A first dAb, a second dAb, the Fc region, the SAM, a third dAb and a fourth dAb;

### Or

(iii) the polypeptide comprises (in C- to N-terminal direction);
A. A dAb and the SAM;
B. A first dAb, the SAM and a second dAb;
C. A first scFv and the SAM;
D. A first scFv, the SAM and a second scFv;
E. A first scFv, the SAM and a first dAb;
F. A first dAb, the Fc region and the SAM;
G. A first scFv, the Fc region and the SAM;
H. A VH, a CH1, the Fc regionand the SAM;
I. A VL, a CL, the Fc region and the SAM;
J. A dAb; the SAM and the Fc region;
K. A scFv; the SAM and the Fc region;
L. A VH, a CH1, the SAM and the Fc region;
M. A VL, a CL, the SAM and the Fc region;
N. A first dAb, a second dAb and the SAM;
O. A VH, a CH1 and the SAM;
P. A VL, a CL and the SAM;
Q. A VH, a CH1, the SAM and a first dAb;
R. A VL, a CL, the SAM and a first dAb;
S. A first dAb, a second dAb, the SAM and a third dAb;
T. A first dAb, a second dAb, the SAM and a first scFv;
U. A first dAb, a second dAb, the SAM, a third dAb and a fourth dAb;
V. A first dAb, the Fc region, the SAM and a second dAb;
W. A first dAb, the Fc region, the SAM and a first scFv;
X. A first dAb, a second dAb, the Fc region and the SAM;
Y. A first dAb, a second dAb, the Fc region, the SAM and a third dAb;
Z. A first dAb, a second dAb, the Fc region, the SAM and a first scFv; or
AA. A first dAb, a second dAb, the Fc region, the SAM, a third dAb and a fourth dAb.

Optionally, any single variable domain or dAb herein is a Nanobody™ or a Camelid VHH (eg, a humanised Camelid VHH).

In an embodiment, each polypeptide of the multimer is paired with a copy of a further polypeptide, wherein the further polypeptide comprises an antibody light chain constant region (eg, a Cκ or a Cλ) that pairs with the Fc of the first polypeptide. In an example, the first polypeptide comprises an antibody VH domain, the further polypeptide comprises an antibody VL domain (eg, a Vκ or a Vλ), wherein the VH and VL form an epitope binding site. In this way, the multimer may be a multimer of Fab-like structures, such as comprising multiple copies of an adalimumab (Humira) or avelumab (Bavencio) binding site as exemplified in the Examples below. All of the antibody domains in such a multimer may, for example, be human, and optionally the SAM is a human domain. When the SAM is a TD (eg, a p53 TD), the multimer comprises a tetramer of the VH/VL epitope binding sites. The first polypeptide or the further polypeptide may comprise a second epitope binding site, for example, wherein the multimer is octavalent. When the 8 binding sites may bind the same antigen; alternatively the 4 VH/VL binding sites bind a first antigen and the other 4 binding sites bind to a second antigen, wherein the antigens are different. Thus, the multimer may be octavalent and bispecific. If the first or further polypeptide comprises yet another antigen binding site, the multimer may be 12-valent (and, eg, monospecific, bispecific or trispecific for antigen binding). If the first or further polypeptide comprises yet another antigen binding site, the multimer may be 16-valent (and, eg, monospecific, bispecific, trispecific or tetraspecific for antigen binding).

The invention further provides:—

A multimer (optionally a tetramer) of a polypeptide according to the invention; optionally wherein the multimer is for medical use. In an example, the medical use herein is the treatment or prevention of a cancer, autoimmune disease or condition or any other disease or condition disclosed herein.
A multimer of a plurality of antibody Fc regions, wherein each Fc is comprised by a respective polypeptide and is unpaired with another Fc region; optionally wherein the multimer is for medical use. Optionally, each polypeptide comprises an epitope binding domain or site as disclosed herein.
A pharmaceutical composition comprising the polypeptide or multimer of the invention.
A nucleic acid encoding a polypeptide of the invention; optionally wherein the nucleic acid is comprised by a eukaryotic cell or a vector.
A method of binding multiple copies of an antigen, the method comprising combining the copies with a multimer of or the composition of the invention, wherein the copies are bound by polypeptides of the multimer, and optionally the method comprising isolating the multimer bound to the antigen copies. In an example, the multimer is contacted with a sample comprising the copies of the antigen and copies of the antigen are sequestered in the sample by binding to the multimer. For example, the multimer is administered to a human or animal patient (or an environment is exposed to the multimer) and antigen copies are sequestered in the human (eg, for said medical use), animal (eg, for said medical use) or environment. For example, the environment is comprised by a soil, water source, waterway or industrial fluid, eg, for environmental remediation, such as where the antigen is comprised by an environmental pollutant or contaminant. In an example, the method is for purifying the sample or for isolating antigen comprised by the sample.
A method of treating or reducing the risk of a disease or condition in a human or animal subject, the method comprising administering the composition of the invention to the subject, wherein multimers comprised by the composition specifically bind to a target antigen in the subject, wherein said binding mediates the treatment or reduction in risk of the disease or condition.
A method of producing a composition comprising a plurality of polypeptides according to the invention, wherein the SAM is a self-associating tetramerisation domain (TD), the method comprising providing eukaryotic host cells according to the invention, culturing the host cells, and allowing expression and secretion from the cells of tetramers of the polypeptides, and optionally isolating or purifying the tetramers.

Paragraphs:

The invention provides the following Paragraphs.

1. A polypeptide (optionally according any polypeptide herein) comprising
(a) An antibody Fc region, wherein the Fc region comprises an antibody CH2 domain and an antibody CH3 domain; and
(b) A self-associating multimerisation domain (SAM);
   wherein the CH2 is devoid of a core hinge CXXC amino acid sequence, wherein X is any amino acid.
   2. The polypeptide of Paragraph 1, wherein each amino acid X is selected from a P, R and S.
   3. The polypeptide of Paragraph 1 or 2, wherein the CH2 is devoid of a complete upper hinge sequence.
   4. The polypeptide of any preceding Paragraph, wherein the CH2 comprises
(a) an APELLGGPSV amino acid sequence, or an PAPELLGGPSV amino acid sequence;
(b) an APPVAGPSV amino acid sequence, or an PAPPVAGPSV amino acid sequence; or
(c) an APEFLGGPSV amino acid sequence, or an PAPEFLGGPSV amino acid sequence.
   5. The polypeptide of any preceding Paragraph, wherein the CH2 and CH3 are
(a) human IgG1 CH2 and CH3 domains;
(b) human IgG2 CH2 and CH3 domains;
(c) human IgG3 CH2 and CH3 domains; or
(d) human IgG4 CH2 and CH3 domains.
   6. The polypeptide of Paragraph 5(a) or (b), wherein CH2 is devoid of a CPPC sequence; or the polypeptide of Paragraph 5(c) wherein the CH2 is devoid of a CPRC sequence; or the polypeptide of Paragraph 5(d) wherein the CH2 is devoid of a CPSC sequence.
   7. The polypeptide of any one of Paragraphs 1 to 4, wherein the CH2 comprises
(a) an APELLGGPSV amino acid sequence;
(b) an EPKSCDKTHT[P]APELLGGPSV amino acid sequence, wherein the bracketed P is optional;
(c) an APPVAGPSV amino acid sequence;
(d) an ERKCCVE[P]APPVAGPSV amino acid sequence, wherein the bracketed P is optional;
(e) an ELKTPLGDTIT[P]APELLGGPSV amino acid sequence, wherein the bracketed P is optional;
(f) an EPKSCDTPPP[P]APELLGGPSV amino acid sequence, wherein the bracketed P is optional; or
(g) an APEFLGGPSV amino acid sequence; or
(h) an ESKYGPP[P]APEFLGGPSV amino acid sequence, wherein the bracketed P is optional.
   8. The polypeptide of any preceding Paragraph, wherein the CH2 is devoid of a sequence selected from CXXC disclosed herein and SEQ ID Nos: 180-182.
   9. The polypeptide of any preceding Paragraph, wherein the polypeptide comprises an antibody variable domain that is capable of specifically binding to a first epitope.
   10. The polypeptide of Paragraph 9, wherein the variable domain selected from an antibody single variable domain, a VH and a VL; or wherein the domain is comprised by an scFv.
   11. The polypeptide of any preceding Paragraph, wherein the Fc region is 3′ of the SAM.
   12. The polypeptide of Paragraph 9, 10 or 11, wherein
(a) the variable domain is 5′ of the SAM and the Fc region is 3′ of the SAM;
(b) the variable domain is 3′ of the SAM and the Fc region is 5′ of the SAM;
(c) the variable domain and Fc are 5′ of the SAM; or
(d) the variable domain and Fc are 3′ of the SAM.
   13. The polypeptide of Paragraph 12 comprising a second variable domain 5′ or 3′ of the SAM, wherein the second variable domain is capable of specifically binding to a second epitope, wherein the first and second epitopes are identical or different.
   14. The polypeptide of paragraph 13, wherein the epitopes are different epitopes of the same antigen, or are epitopes of different antigens.
   15. The polypeptide of Paragraph 13 or 14, wherein the second variable domain is selected from an antibody single variable domain, a VH and a VL; or wherein the domain is comprised by an scFv.
   16. The polypeptide of Paragraph 15, wherein the second variable domain is an antibody single variable domain or an antibody constant domain.
   17. The polypeptide of any preceding Paragraph, wherein the SAM is a self-associating tetramerisation domain (TD).
   18. The polypeptide of Paragraph 17, wherein the TD is a p53, p63 or p73 TD or a homologue or orthologue thereof; or wherein the TD is a NHR2 TD or a homologue or orthologue thereof.
   19. The polypeptide of Paragraph 17 or 18, wherein the TD comprises an amino acid sequence that is at least 80% identical to SEQ ID NO: 10 or 126.
   20. The polypeptide of any preceding Paragraph, comprising an antibody CH1 constant domain, optionally a CH1-CH2-CH3, wherein the CH2 and CH3 are comprised by said Fc region.
   21. The polypeptide of any preceding Paragraph, wherein the polypeptide (first polypeptide) comprises or consists of (in N- to C-terminal direction);
A. A first antibody single variable domain (dAb), an optional linker and said SAM;
B. A first antibody single variable domain, an optional linker, said SAM and a second antibody single variable domain;
C. A first scFv, an optional linker and said SAM;
D. A first scFv, an optional linker, said SAM and a second scFv;
E. A first antibody single variable domain, an optional linker, said SAM and a first scFv;
F. A first scFv, an optional linker, said SAM and a first antibody single variable domain;
G. A first antibody variable domain, an optional first linker, a first antibody constant domain, a second optional linker and said SAM;
H. Said SAM, an optional linker and a first antibody single variable domain;
I. Said SAM, an optional linker and a first scFv;
J. Said SAM, an optional linker, a first antibody constant domain, a second optional linker and a first antibody variable domain; or
K. Said SAM, an optional linker, a first antibody variable domain, a second optional linker and a first antibody constant domain.
   22. The polypeptide of any of Paragraphs 1 to 20, wherein
   (i) the polypeptide comprises (in N- to C-terminal direction);
A. A dAb and the self-associating multimersiation domain (SAM);
B. A first dAb, the SAM and a second dAb;
C. A first scFv and the SAM;
D. A first scFv, the SAM and a second scFv;
E. A first scFv, the SAM and a first dAb;
F. A first dAb, the Fc region and the SAM;
G. A first scFv, the Fc region and the SAM;
H. A VH, a CH1, the Fc region and the SAM;
I. A VL, a CL, the Fc region and the SAM;
J. A dAb; the SAM and the Fc region;
K. A scFv; the SAM and the Fc region;
L. A VH, a CH1, the SAM and the Fc region;
M. A VL, a CL, the SAM and the Fc region;
N. A first dAb, a second dAb and the SAM;
O. A VH, a CH1 and the SAM;
P. A VL, a CL and the SAM;
Q. A VH, a CH1, the SAM and a first dAb;
R. A VL, a CL, the SAM and a first dAb;
S. A first dAb, a second dAb, the SAM and a third dAb;
T. A first dAb, a second dAb, the SAM and a first scFv;
U. A first dAb, a second dAb, the SAM, a third dAb and a fourth dAb;
V. A first dAb, the Fc region, the SAM and a second dAb;
W. A first dAb, the Fc region, the SAM and a first scFv;
X. A first dAb, a second dAb, the Fc region and the SAM;
Y. A first dAb, a second dAb, the Fc region, the SAM and a third dAb;
Z. A first dAb, a second dAb, the Fc region, the SAM and a first scFv; or
AA. A first dAb, a second dAb, the Fc region, the SAM, a third dAb and a fourth dAb;

### Or

(ii) the polypeptide comprises (in C- to N-terminal direction);
A. A dAb and a self-associating multimersiation domain (SAM);
B. A first dAb, the SAM and a second dAb;
C. A first scFv and the SAM;
D. A first scFv, the SAM and a second scFv;
E. A first scFv, the SAM and a first dAb;
F. A first dAb, the Fc region and the SAM;
G. A first scFv, the Fc region and the SAM;
H. A VH, a CH1, the Fc regionand the SAM;
I. A VL, a CL, the Fc region and the SAM;
J. A dAb; the SAM and the Fc region;
K. A scFv; the SAM and the Fc region;
L. A VH, a CH1, the SAM and the Fc region;
M. A VL, a CL, the SAM and the Fc region;
N. A first dAb, a second dAb and the SAM;
O. A VH, a CH1 and the SAM;
P. A VL, a CL and the SAM;
Q. A VH, a CH1, the SAM and a first dAb;
R. A VL, a CL, the SAM and a first dAb;
S. A first dAb, a second dAb, the SAM and a third dAb;
T. A first dAb, a second dAb, the SAM and a first scFv;
U. A first dAb, a second dAb, the SAM, a third dAb and a fourth dAb;
V. A first dAb, the Fc region, the SAM and a second dAb;
W. A first dAb, the Fc region, the SAM and a first scFv;
X. A first dAb, a second dAb, the Fc region and the SAM;
Y. A first dAb, a second dAb, the Fc region, the SAM and a third dAb;
Z. A first dAb, a second dAb, the Fc region, the SAM and a first scFv; or
AA. A first dAb, a second dAb, the Fc region, the SAM, a third dAb and a fourth dAb.
   23. The polypeptide of Paragraph 21B, 21D, (i) 22B, (i) 22D, (i) 22N, (i) 22S, (i) 22T, (i) 22U, (i) 22X, (i) 22Y, (i) 22Z, (i) 22AA, (ii) 22B, (ii) 22D, (ii) 22N, (ii) 22S, (ii) 22T, (ii) 22U, (ii) 22X, (ii) 22Y, (ii) 22Z or (ii) 22AA wherein the single variable domains (dAbs) are identical; or wherein the scFvs are identical.
   24. The polypeptide of Paragraph 21B, 21D, (i) 22B, (i) 22D, (i) 22N, (i) 22S, (i) 22T, (i) 22U, (i) 22X, (i) 22Y, (i) 22Z, (i) 22AA, (ii) 22B, (ii) 22D, (ii) 22N, (ii) 22S, (ii) 22T, (ii) 22U, (ii) 22X, (ii) 22Y, (ii) 22Z or (ii) 22AA wherein the single variable domains are different; or wherein the scFvs are different.
   25. The polypeptide of Paragraph 21G, 21J, 21K, (i) 22H, (i) 221, (i) 22L, (i) 22M, (i) 220, (i) 22P, (i) 22Q, (i) 22R, (ii) 22H, (ii) 221, (ii) 22L, (ii) 22M, (ii) 220, (ii) 22P, (ii) 22Q or (ii) 22R wherein (i) the first variable domain is a VH domain and the first constant domain is a CH1 domain, and optionally the polypeptide is associated with a second polypeptide, wherein the second polypeptide comprises an antibody CL constant domain that is paired with the CH1 domain; (ii) the first variable domain is a VH domain and the first constant domain is a CL domain, and optionally the polypeptide is associated with a second polypeptide, wherein the second polypeptide comprises an antibody CH1 constant domain that is paired with the CL domain; (iii) the first variable domain is a VL domain and the first constant domain is a CH1 domain, and optionally the polypeptide is associated with a second polypeptide, wherein the second polypeptide comprises an antibody CL constant domain that is paired with the CH1 domain; or (iv) the first variable domain is a VL domain and the first constant domain is a CL domain, and optionally the polypeptide is associated with a second polypeptide, wherein the second polypeptide comprises an antibody CH1 constant domain that is paired with the CL domain.
   26. The polypeptide of any one of Paragraphs 21 to 25, comprising an antibody Fc region or antibody single variable domain between (v) the first variable domain or scFv and (vi) the SAM, wherein the Fc comprises a CH2 and a CH3.
   27. The polypeptide of any one of Paragraphs 21 to 26, comprising the Fc region or an antibody single variable domain between (i) the SAM and (ii) the most C-terminal variable domain.
   28. The polypeptide of any one of Paragraphs 21 to 27, comprising in N- to C-terminal direction the antibody Fc region and the most N-terminal variable domain or scFv.
   29. The polypeptide of any one of Paragraphs 21 to 28, comprising in N- to C-terminal direction the SAM and the antibody Fc region.
   30. The polypeptide of any one of Paragraphs 21 to 29, wherein the first or each linker is a (G₄S)ₙ linker, wherein n=1, 2, 3, 4, 5, 6, 7, 8, 9 or 10.
   31. The polypeptide of any preceding Paragraph, wherein each domain and SAM is a human domain and SAM respectively.
   32. The polypeptide of any preceding Paragraph, wherein the polypeptide comprises binding specificity for more than one antigen, optionally 2, 3 or 4 different antigens.
   33. A multimer (optionally a tetramer) of a polypeptide according to any preceding Paragraph.
   34. A multimer of a plurality of antibody Fc regions, wherein each Fc is comprised by a respective polypeptide and is unpaired with another Fc region.
   35. The multimer of Paragraph 34, wherein the multimer is a polypeptide tetramer comprising at least 4 Fc regions.
   36. The multimer of Paragraph 34 or 35, wherein each Fc region is identical.
   37. The multimer of any one of Paragraphs, wherein each Fc comprise a CH2 and a CH3, wherein each CH2 is a CH2 as recited in any one of Paragraphs 1 to 32.
   38. The polypeptide or multimer of any preceding Paragraph, comprising eukaryotic cell glycosylation.
   39. The polypeptide or multimer of Paragraph 38, wherein the cell is a HEK293, CHO or Cos cell.
   40. The polypeptide or multimer of any preceding Paragraph for medical use.
   41. A pharmaceutical composition comprising the polypeptide or multimer of any preceding Paragraph.
   42. A nucleic acid encoding a polypeptide of any one of Paragraphs 1 to 32 and 38 to 40.
   43. A eukaryotic cell or vector comprising the nucleic acid of Paragraph 42.
   44. A method of binding multiple copies of an antigen, the method comprising combining the copies with a multimer of any one of Paragraphs 33 to 40, wherein the copies are bound by polypeptides of the multimer, and optionally the method comprising isolating the multimer bound to the antigen copies.
   45. The method of Paragraph 37 wherein the method is a diagnostic method for detecting the presence of a substance in a sample, wherein the substance comprises the antigen, the method comprising providing the sample (eg, a bodily fluid, food, food ingredient, beverage, beverage ingredient, soil or forensic sample), mixing the sample with multimers according to any one of Paragraphs 33 to 40 and detecting the binding of multimers to the antigen in the sample.
   46. A method of treating or reducing the risk of a disease or condition in a human or animal subject, the method comprising administering the composition of Paragraph 41 to the subject, wherein multimers comprised by the composition specifically bind to a target antigen in the subject, wherein said binding mediates the treatment or reduction in risk.
   47. The method of Paragraph 46, wherein the antigen is an immune checkpoint antigen (eg, PD-L1, PD-1 or CTLA4); or wherein the antigen is TNF alpha or IL-17A.
   48. The method of Paragraph 46, wherein the antigen mediates the disease or condition in the subject; and optionally wherein the binding antagonises the antigen.
   49. A composition comprising a plurality of polypeptides according to any one of Paragraphs 1 to 32 and 38 to 40, wherein at least 90% of the polypeptides are comprised by tetramers of said polypeptides.
   50. The composition of Paragraph 49, wherein at least 98% of the polypeptides are comprised by tetramers of said polypeptides.
   51. The composition of Paragraph 49 or 50, wherein the remaining polypeptides are selected from one or more of polypeptide monomers, dimers and trimers.
   52. A method of producing a composition (optionally a composition according to any one of Paragraphs 49 to 51) comprising a plurality of polypeptides according to any one of Paragraphs 1 to
   32 and 38 to 40, the method comprising providing eukaryotic host cells according to Paragraph 34, culturing the host cells, and allowing expression and secretion from the cells of tetramers of the polypeptides, and optionally isolating or purifying the tetramers.

Concepts:

In certain embodiments, the invention is useful for providing multimers for treating cancer in humans or animals. In this respect, it may be useful to use the multimers to target tumours by binding to tumour-associated antigen and/or to bind to T-cells to modulate their activity. For example the multimers may bind to an antigen on T regulatory cells (Tregs) to downregulate their activity. Additionally or alternatively, the multimers may bind to T effector (Teff) cells to upregulate their activity. The provision of an antibody Fc region in the polypeptides of multimers may be advantageous for providing Fc effector functions and/or cytotoxicity for killing tumour cells. In one advantageous configuration, the invention exploits the ability to provide multiple identical antigen or epitope binding sites that can be used to bind to several copies of the same antigen or epitope on tumour cells, thereby providing for an avidity affect wherein the multimers bind preferentially to tumour cells over any non-target or normal cells, since the former surface-express more copies of the antigen than normal cells. In one configuration, when the multimer also comprises binding sites for an immune checkpoint regulator. In one example the regulator is an immune checkpoint inhibitor and the binding sites antagonise the inhibitor. This is useful, for example when the inhibitor is expressed by Teff cells, for upregulating Teff activity in the vicinity of tumour cells that are targeted by the multimer (eg, by binding TAA on the tumour cells). In another example the regulator is an immune checkpoint stimulator and the binding sites agonise the inhibitor. This is useful, for example when the inhibitor is expressed by Teff cells, for upregulating Teff activity in the vicinity of tumour cells that are targeted by the multimer (eg, by binding TAA on the tumour cells). Thus, upregulation of T-cell activity may be stimulated in the vicinity of tumour cells, rather in the vicinity of non-target (eg, normal or non-cancerous) cells. To this end, the invention provides the following Concepts.

1. A polypeptide comprising a self-associating multimerisation domain (SAM), a first antigen binding site and a second antigen binding site, wherein the first site specifically binds to a first antigen or epitope, and the second binding site specifically binds to a second antigen or epitope, wherein each antigen or epitope is a tumour-associated antigen (TAA) or epitope, or an immune checkpoint regulator (eg, inhibitor) antigen or epitope.
2. The polypeptide of Concept 1, wherein the first antigen is a TAA and the second antigen is an immune checkpoint regulator (eg, inhibitor).
3. The polypeptide of Concept 1, wherein the first antigen is a TAA and the second antigen is a TAA.
4. The polypeptide of Concept 1, wherein the first antigen is an immune checkpoint inhibitor and the second antigen is an immune checkpoint regulator (eg, inhibitor).
5. The polypeptide of Concept 3 or 4, wherein the binding sites are capable of specifically binding to the same epitope of the same antigen.
6. The polypeptide of Concept 3 or 4, wherein the binding sites are capable of specifically binding to different epitopes of the same antigen.
7. The polypeptide of any preceding Concept, wherein the first antigen is selected from 4-1BB, 4-1BBL, CD28, OX40, OX40L, ICOS, ICOSL, GITR, CD40, CD27, CD27L, CD40L, LIGHT, CD70, CD80, CD86, HER2, HER3, PSMA, WT1, MUC1, LMP2, EGFRvIII, MAGE A3, GD2, CEA, Melan a/MART1, Bcr-Abl, Survivin, PSA, hTERT, EphA2, PAP, EpCAM, ERG, PAX3, ALK, Androgen receptor, Cyclin B1, RhoC, GD3, PSCA, PAX5, LCK, VEGFR2, MAD CT-1, FAP, MAD CT-2, PDGFR-beta, Fos related antigen 1, NY-BR-1, ETV6-AML, RGS5, SART3, SSX2, XAGE-1, STn, PAP and BCMA.
8. The polypeptide of any preceding Concept, wherein the second antigen is selected from PDL1, PD1, CTLA4, BTLA, KIR, LAG3, TIM3, A2aR, HVEM, GAL9, VISTA, SIRPa, CD47, CD160, CD155, IDO, CEACAMI, 2B4, CD48 and TIGIT.
9. The polypeptide of any preceding Concept, wherein the polypeptide comprises a third antigen binding site that is capable of specifically binding to a third antigen or epitope.
10. The polypeptide of Concept 9, wherein the third antigen is a TAA.
11. The polypeptide of Concept 9, wherein the third antigen is an immune checkpoint regulator (eg, inhibitor).
12. The polypeptide of any one of Concepts 9 to 11, wherein the third antigen is the same as the first or second antigen.
13. The polypeptide of any one of Concepts 9 to 12, wherein the third antigen is CD3 (eg, CD3e) or CD28.
14. The polypeptide of any one of Concepts 9 to 13, wherein the polypeptide comprises a fourth antigen binding site that is capable of specifically binding to a fourth antigen or epitope.
15. The polypeptide of Concept 14, wherein the fourth antigen is a TAA.
16. The polypeptide of Concept 14, wherein the fourth antigen is an immune checkpoint regulator (eg, inhibitor).
17. The polypeptide of any preceding Concept, wherein the polypeptide is according to any polypeptide disclosed herein.

In an example, said first dAb or first scFv of the polypeptide herein is the first antigen binding site of these Concepts; and optionally when a further dAb or scFv binding site is present this is the second antigen binding site of the Concepts.

In an example, said first dAb or first scFv of the polypeptide herein is the second antigen binding site of these Concepts; and optionally when a further dAb or scFv binding site is present this is the first antigen binding site of the Concepts.

18. A multimer of a polypeptide of any preceding Concept.
19. The multimer of Concept 18 for administration to a human or animal subject for targeting of an immune checkpoint inhibitor and an immune co-stimulatory molecule for the treatment of cancer.
20. A method of treating a cancer in a human or animal subject, the method comprising administering the multimer of claim 18 to the subject.

Clauses

In a configuration, the invention provides the following Clauses.

1. A protein multimer of at least first, second, third and fourth copies of an effector domain (eg, a protein domain) or a peptide, wherein the multimer is multimerised by first, second, third and fourth self-associating tetramerisation domains (TDs) which are associated together, wherein each tetramerisation domain is comprised by a respective engineered polypeptide comprising one or morecopies of said protein domain or peptide.
2. The multimer of Clause 1, wherein the multimer is a tetramer, octamer, dodecamer, hexadecamer or 20-mer (eg, tetramer or an octamer) of said domain or peptide.
3. The multimer of any Clause 1 or 2, comprising a tetramer, octamer, dodecamer, hexadecamer or 20-mer (eg, tetramer or an octamer) of an immunoglobulin superfamily binding site.
4. The multimer of Clause 3, wherein the binding site comprises a first variable domain paired with a second variable domain.
5. The multimer of any preceding Clause, wherein each polypeptide comprises first and second copies of said protein domain or peptide, wherein the polypeptide comprises in (N- to C-terminal direction) (i) a first of said copies—TD—the second of said copies; (ii) TD—and the first and second copies; or (iii) said first and second copies—TD.
6. The multimer of any preceding Clause, wherein the TDs are NHR2 TDs and the domain or peptide is not a NHR2 domain or peptide; or wherein the TDs are p53 TDs and the domain or peptide is not a p53 domain or peptide.
7. The multimer of any preceding Clause, wherein the engineered polypeptide comprises one or more copies of a second type of protein domain or peptide, wherein the second type of protein domain or peptide is different from the first protein domain or peptide.
8. The multimer of any preceding Clause, wherein the domains are immunoglobulin superfamily domains.
9. The multimer of any preceding Clause, wherein the domain or peptide is an antibody variable or constant domain, a TCR variable or constant domain, an incretin, an insulin peptide, or a hormone peptide.
10. The multimer of any preceding Clause, wherein the multimer comprises first, second, third and fourth identical copies of a said engineered polypeptide, the polypeptide comprising a TD and one (but no more than one), two (but no more than two) or more copies of the said protein domain or peptide.
11. The multimer of any preceding Clause, wherein the engineered polypeptide comprises an antibody or TCR variable domain (V1) and a NHR2 TD.
12. The multimer of Clause 11, wherein the polypeptide comprises (in N- to C-terminal direction) (i) V1-an optional linker-NHR2 TD; (ii) V1-an optional linker-NHR2 TD-optional linker-V2; or (iii) V1-an optional linker-V2—optional linker—NHR2 TD, wherein V1 and V2 are TCR variable domains and are the same or different, or wherein V1 and V2 are antibody variable domains and are the same or different.
13. The multimer of Clause 12, wherein V1 and V2 are antibody single variable domains.
14. The multimer of Clause 11, wherein each engineered polypeptide comprises (in N- to C-terminal direction) V1-an optional linker—TD, wherein V1 is an antibody or TCR variable domain and each engineered polypeptide is paired with a respective second engineered polypeptide that comprises V2, wherein V2 is a an antibody or TCR variable domain respectively that pairs with V1 to form an antigen or pMHC binding site, and optionally one polypeptide comprises an antibody Fc, or comprises antibody CH1 and the other polypeptide comprises an antibody CL that pairs with the CH1.
15. The multimer of any preceding Clause, wherein the TD comprises (i) an amino acid sequence identical to SEQ ID NO: 10 or 126 or at least 80% identical thereto; or (ii) an amino acid sequence identical to SEQ ID NO: 120 or 123 or at least 80% identical thereto.
16. The multimer of any preceding Clause, wherein the multimer comprises a tetramer, octamer, dodecamer, hexadecamer or 20-mer (eg, tetramer or an octamer) of an antigen binding site of an antibody selected from the group consisting of ReoPro™; Abciximab; Rituxan™; Rituximab; Zenapax™; Daclizumab; Simulect™; Basiliximab; Synagis™; Palivizumab; Remicade™; Infliximab; Herceptin™; Mylotarg™; Gemtuzumab; Campath™; Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara™; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; Kadcyla™; Ado-trastuzumab; Keytruda™, Opdivo™, Gazyva™ and Obinutuzumab.
17. An isolated tetramer, octamer, dodecamer, hexadecamer or 20-mer of a TCR binding site, insulin peptide, incretin peptide or peptide hormone; or a plurality of said tetramers or octamers.
18. The multimer, tetramer, octamer, dodecamer, hexadecamer or 20-mer (eg, tetramer or an octamer) of any preceding Clause, wherein the mulitmer, tetramer, octamer, dodecamer, hexadecamer or 20-mer is
(a) soluble in aqueous solution;
(b) secretable from a eukaryotic cell; and/or
(c) an expression product of a eukaryotic cell.
   19. A tetramer, octamer, dodecamer, hexadecamer or 20-mer (eg, tetramer or an octamer) of
(a) TCR V domains or TCR binding sites, wherein the tetramer, octamer, dodecamer, hexadecamer or 20-mer is soluble in aqueous solution;
(b) antibody single variable domains, wherein the tetramer, octamer, dodecamer, hexadecamer or 20-mer is soluble in aqueous solution;
(c) TCR V domains or TCR binding sites, wherein the tetramer, octamer, dodecamer, hexadecamer or 20-mer is capable of being intracellularly and/or extracellularly expressed by HEK293 cells; or
(d) antibody variable domains, wherein the tetramer, octamer, dodecamer, hexadecamer or 20-mer is capable of being intracellularly and/or extracellularly expressed by HEK293 cells.
   20. The multimer, tetramer, octamer, dodecamer, hexadecamer or 20-mer of any preceding Clause, wherein the tetramer, octamer, dodecamer, hexadecamer or 20-mer is bi-specific for antigen or pMHC binding.
   21. The multimer, tetramer, octamer, dodecamer, hexadecamer or 20-mer of any preceding Clause, wherein the domains are identical.
   22. The multimer, tetramer, octamer, dodecamer, hexadecamer or 20-mer of any preceding Clause, wherein the multimer, tetramer, octamer, dodecamer, hexadecamer or 20-mer comprises eukaryotic cell glycosylation.
   23. The multimer, tetramer, octamer, dodecamer, hexadecamer or 20-mer of Clause 22, wherein the cell is a HEK293 cell.
   24. A plurality of multimers, tetramers, octamers, dodecamers, hexadecamers or 20-mer s (eg, tetramers or octamers) of any preceding Clause.
   25. A pharmaceutical composition comprising the multimer(s), tetramer(s), octamer(s), dodecamer(s), hexadecamer(s) or 20-mer (s) (eg, tetramer(s) or octamer(s)) of any preceding Clause and a pharmaceutically acceptable carrier, diluent or excipient.
   26. A cosmetic, foodstuff, beverage, cleaning product, detergent comprising the multimer(s), tetramer(s), octamer(s), dodecamer(s), hexadecamer(s) or 20-mer (s) (eg, tetramer(s) or octamer(s)) of any one of Clauses 1 to 24.
   27. A said engineered (and optionally isolated) polypeptide or a monomer (optionally isolated) of a multimer, tetramer, octamer, dodecamer, hexadecamer or 20-mer of any preceding Clause.
   28. An engineered (and optionally isolated) polypeptide (P1) which comprises (in N- to C-terminal direction):—
   (a) TCR V1-TCR C1—antibody CH1—optional linker—TD, wherein
(i) V1 is a Vα and C1 is a Cα;
(ii) V1 is a Vβ and C1 is a Cβ;
(iii) V1 is a Vγ and C1 is a Cγ; or
(iv) V1 is a Vδ and C1 is a Cδ;
   or
      (b) TCR V1—antibody CH1—optional linker—TD, wherein
(i) V1 is a Vα;
(ii) V1 is a Vβ;
(iii) V1 is a Vγ; or
(iv) V1 is a Vδ;
   or
      (c) antibody V1—antibody CH1—optional linker—TD, wherein
(i) V1 is a VH; or
(ii) V1 is a VL;
   or
      (d) antibody V1—optional antibody CH1—antibody Fc—optional linker—TD, wherein
(i) V1 is a VH; or
(ii) V1 is a VL;
   or
      (e) antibody V1—antibody CL—optional linker—TD, wherein
(i) V1 is a VH; or
(ii) V1 is a VL;
   or
      (f) TCR V1-TCR C1—optional linker—TD, wherein
(i) V1 is a Vα and C1 is a Cα;
(ii) V1 is a Vβ and C1 is a Cβ;
(iii) V1 is a Vγ and C1 is a Cγ; or
(iv) V1 is a Vδ and C1 is a Cδ.
   29. The polypeptide of Clause 28, wherein the engineered polypeptide P1 is paired with a further polypeptide (P2), wherein P2 comprises (in N- to C-terminal direction):—
   (g) TCR V2-TCR C2—antibody CL, wherein P1 is according to (a) recited in Clause 28 and
(i) V2 is a Vα and C2 is a Cα when P1 is according to (a)(ii);
(ii) V2 is a Vβ and C2 is a Cβ when P1 is according to (a)(i);
(iii) V2 is a Vγ and C2 is a Cγ when P1 is according to (a)(iv); or
(iv) V2 is a Vδ and C2 is a Cδ when P1 is according to (a)(iii);
   or
      (h) TCR V2—antibody CL, wherein P1 is according to (b) recited in Clause 28 and
(i) V2 is a Vα when P1 is according to (b)(ii);
(ii) V2 is a Vβ when P1 is according to (b)(i);
(iii) V2 is a Vγ when P1 is according to (b)(iv); or
(iv) V2 is a Vδ when P1 is according to (b)(iii);
   or
      (i) Antibody V2-CL, wherein P1 is according to (c) recited in Clause 28 and
(i) V2 is a VH when P1 is according to (c)(ii); or
(ii) V2 is a VL when P1 is according to (c)(i);
   or
      (j) Antibody V2—optional CL, wherein P1 is according to (d) recited in Clause 28 and
(i) V2 is a VH when P1 is according to (d)(ii); or
(ii) V2 is a VL when P1 is according to (d)(i);
   or
      (k) Antibody V2-CH1, wherein P1 is according to (e) recited in Clause 28 and
(i) V2 is a VH when P1 is according to (e)(ii); or
(ii) V2 is a VL when P1 is according to (e)(i);
   or
      (l) TCR V2-TCR C2, wherein P1 is according to (f) recited in Clause 28 and
(i) V2 is a Vα and C2 is a Cα when P1 is according to (f)(ii);
(ii) V2 is a Vβ and C2 is a Cβ when P1 is according to (f)(i);
(iii) V2 is a V₇ and C2 is a Cγ when P1 is according to (f)(iii); or
(iv) V2 is a Vδ and C2 is a Cδ when P1 is according to (f)(iv).
   30. A multimer of P1 as defined in Clause 28; or of P1 paired with P2 as defined in Clause 29; or a plurality of said multimers, optionally wherein the multimer is according to any one of Clauses 1 to 24.
   31. A nucleic acid encoding an engineered polypeptide or monomer of any one of Clauses 27 to
   29, optionally wherein the nucleic acid is comprised by an expression vector for expressing the polypeptide.
   32. A eukaryotic host cell comprising the nucleic acid or vector of Clause 31 for intracellular and/or secreted expression of the multimer, tetramer, octamer, dodecamer, hexadecamer or 20-mer (eg, tetramer, octamer), engineered polypeptide or monomer of any one of Clauses 1 to 24.
   33. Use of a nucleic acid or vector according to Clause 31 in a method of manufacture of protein multimers for producing intracellularly expressed and/or secreted multimers, wherein the method comprises expressing the multimers in and/or secreting the multimers from eukaryotic cells comprising the nucleic acid or vector.
   34. Use of a nucleic acid or vector according to Clause 31 in a method of manufacture of protein multimers for producing glycosylated multimers in eukaryotic cells comprising the nucleic acid or vector.
   35. A mixture comprising (i) a eukaryotic cell line encoding an engineered polypeptide according to any one of Clauses 27 to 29; and (ii) multimers, tetramers, octamers, dodecamers, hexadecamers or 20-mer s (eg, tetramers or octamers) as defined in any one of Clauses 1 to 24.
   36. The mixture of Clause 35, wherein the cell line is in a medium comprising secretion products of the cells, wherein the secretion products comprise said multimers, tetramers, octamers, dodecamers, hexadecamers or 20-mer s (eg, tetramers or octamers).
   37. The multimer, tetramer, octamer, dodecamer, hexadecamer or 20-mer (eg, tetramer, octamer) of any one of Clauses 1 to 24 for medical use.
   38. A method producing
(a) TCR V domain multimers, the method comprising the soluble and/or intracellular expression of TCR V-NHR2 TD or TCR V-p53 TD fusion proteins expressed in eukaryotic cells, the method optionally comprising isolating a plurality of said multimers;
(b) antibody V domain multimers, the method comprising the soluble and/or intracellular expression of antibody V-NHR2 TD or V-p53 TD fusion proteins expressed in eukaryotic cells, the method optionally comprising isolating a plurality of said multimers;
(c) incretin peptide multimers, the method comprising the soluble and/or intracellular expression of incretin peptide-NHR2 TD or incretin peptide-p53 TD fusion proteins expressed in eukaryotic cells, such as HEK293T cells; the method optionally comprising isolating a plurality of said multimers; or
(d) peptide hormone multimers, the method comprising the soluble and/or intracellular expression of peptide hormone-NHR2 TD or peptide hormone-p53 TD fusion proteins expressed in eukaryotic cells, such as HEK293T cells; the method optionally comprising isolating a plurality of said multimers.
   39. Use of self-associating tetramerisation domains (TD) in a method of the manufacture of a tetramer of polypeptides, for producing a higher yield of tetramers versus monomer and/or dimer polypeptides.
   40. Use of an engineered polypeptide in a method of the manufacture of a tetramer of a polypeptide comprising multiple copies of a protein domain or peptide, for producing a higher yield of tetramers versus monomer and/or dimer polypeptides, wherein the engineered polypeptide comprises one or more copies of said protein domain or peptide and further comprises a self-associating tetramerisation domains (TD).
   41. Use of self-associating tetramerisation domains (TD) in a method of the manufacture of a tetramer of a polypeptide, for producing a plurality of tetramers that are not in mixture with monomers, dimers or trimers.
   42. Use of an engineered polypeptide in a method of the manufacture of a tetramer of a polypeptide comprising multiple copies of a protein domain or peptide, for producing a plurality of tetramers that are not in mixture with monomers, dimers or trimers, wherein the engineered polypeptide comprises one or more copies of said protein domain or peptide and further comprises a self-associating tetramerisation domains (TD).
   43. The use of any one of Clauses 39 to 42, wherein the yield of tetramers is at least 10× the yield of monomers and/or dimers.
   44. The use of any one of Clauses 39 to 43, wherein the ratio of tetramers produced:monomers and/or dimers produced in the method is at least 90:10.
   45. The use of any one of Clauses 39 to 44, wherein each monomer has a size of no more than 40 kDa.
   46. The use of any one of Clauses 39 to 45, wherein each tetramer has a size of no more than 150 kDa.
   47. The use of any one of Clauses 39 to 46, wherein the method comprises expressing the tetramers from a eukaryotic cell line.
   48. A multivalent heterodimeric soluble T cell receptor capable of binding pMHC complex comprising:
(a) TCR extracellular domains;
(b) immunoglobulin constant domains; and
(c) an NHR2 multimerisation domain of ETO.
   49. A multimeric immunoglobulin, comprising
   (i) immunoglobulin variable domains; and
   (ii) an NHR2 multimerisation domain of ETO.
   50. A method for assembling a soluble, multimeric polypeptide, comprising:
(a) providing a monomer of the said multimeric polypeptide, fused to an NHR2 domain of ETO; and
(b) causing multiple copies of said monomer to associate, thereby obtaining a multimeric, soluble polypeptide.

In any disclosure herein, the or each constant region or domain, the CH2, the CH3, the CH2 and CH3 or the Fc is respectively a constant region or domain, the CH2, the CH3, the CH2 and CH3 or the Fc of a human constant region. For example, the constant region is selected from the group IGHA1*01, IGHA1*02, IGHA1*03, IGHA2*01, IGHA2*02, IGHA2*03, IGHD*01, IGHD*02, IGHE*01, IGHE*02, IGHE*03, IGHE*04, IGHEP1*01, IGHEP1*02, IGHEP1*03, IGHEP1*04, IGHG1*01, IGHG1*02, IGHG1*03, IGHG1*04, IGHG1*05, IGHG1*06, IGHG1*07, IGHG1*08, IGHG1*09, IGHG1*10, IGHG1*11, IGHG1*12, IGHG1*13, IGHG1*14, IGHG2*01, IGHG2*02, IGHG2*03, IGHG2*04, IGHG2*05, IGHG2*06, IGHG2*07, IGHG2*08, IGHG2*09, IGHG2*10, IGHG2*11, IGHG2*12, IGHG2*13, IGHG2*14, IGHG2*15, IGHG2*16, IGHG2*17, IGHG3*01, IGHG3*02, IGHG3*03, IGHG3*04, IGHG3*05, IGHG3*06, IGHG3*07, IGHG3*08, IGHG3*09, IGHG3*10, IGHG3*11, IGHG3*12, IGHG3*13, IGHG3*14, IGHG3*15, IGHG3*16, IGHG3*17, IGHG3*18, IGHG3*19, IGHG3*20, IGHG3*21, IGHG3*22, IGHG3*23, IGHG3*24, IGHG3*25, IGHG3*26, IGHG3*27, IGHG3*28, IGHG3*29, IGHG4*01, IGHG4*02, IGHG4*03, IGHG4*04, IGHG4*05, IGHG4*06, IGHG4*07, IGHG4*08, IGHGP*01, IGHGP*02, IGHGP*03, IGHM*01, IGHM*02, IGHM*03 and IGHM*04 (eg, the constant region is a *01 allele listed in said group, preferably the constant region is a human IGHG1*01 or IGHM*01 constant region). In an alternative, the constant region is a non-human (eg, mammal, rodent, mouse, rat, dog, cat or horse) constant region, such as a homologue of a human constant region listed in said group.

The polypeptide, in one embodiment, comprises (in N- to C-terminal direction) a first antigen binding site (eg, a dAb), an antibody CH1 (eg, human IgG1 CH1), a hinge sequence comprising a lower hinge and devoid of a core hinge region (and optionally devoid of an upper hinge region), an antibody Fc region and a SAM (eg, a TD, such as a p53 TD). For example, the core hinge region sequence is a CXXC amino acid sequence. The polypeptide may comprise another antigen binding site (eg a dAb or scFv) between the first binding site and the CH1, between the Fc and SAM and/or C-terminal to the SAM. Optionally, the multimer comprises a plurality (eg, 4 copies) of such polypeptide, for example wherein each polypeptide is paired with a further polypeptide comprising (in N- to C-terminal direction) a second antigen binding site (eg, a dAb), an antibody CL (eg, a human Cκ) and optionally a third antigen binding site. Optionally the binding sites have the same antigen specificity (eg, all bind TNF alpha). In another option, the first and second (and optionally said another binding site) bind to different antigens. The or each binding site can bind any antigen disclosed herein, eg, each binding site binds TNF alpha (as shown in Example 17). In another example, the first antigen binding site is a VH of an antigen binding site of a predetermined antibody that specifically binds to the antigen (and the CH1 is optionally the CH1 of the antibody), and the second binding site of the further polypeptide is a VL of the antigen binding site of the predetermined antibody (and the CL is optionally the CL of the antibody), wherein the VH and VL pair to form a VH/VL binding site which has binding specificity for the antigen. The predetermined antibody may be a marketed antibody, for example, as shown in Example 19. For example, the VH/VL binding site specifically binds to CTLA-4, eg, wherein the predetermined antibody is ipilimumab (or Yervoy™). For example, the VH/VL binding site specifically binds to TNF alpha, eg, wherein the predetermined antibody is adalimumab, golimumab, infliximab (or Humira™, Simponi™ or Remicade™). For example, the VH/VL binding site specifically binds to PD-L1, eg, wherein the predetermined antibody is avelumab (or Bavencio™) or atezolizumab (or Tecentriq™). For example, the VH/VL binding site specifically binds to PD-1, eg, wherein the predetermined antibody is nivolumab (or Opdivo™) or pembrolizumab (or Keytruda™). For example, the VH/VL binding site specifically binds to VEGF, eg, wherein the predetermined antibody is bevacizumab (or Avastin™) or ranibizumab (or Lucentis™). In another example, the polypeptide comprises (in N- to C-terminal direction) a first VEGF binding site, an optional second VEGF binding site, an antibody CH1 (eg, human IgG1 CH1), a hinge sequence comprising a lower hinge and devoid of a core hinge region (and optionally devoid of an upper hinge region), an antibody Fc region and a SAM (eg, a TD, such as a p53 TD). In an example, the first binding site is a Ig domain 2 from VEGFR1 and the second binding site is Ig domain 3 from VEGFR2 (as shown in Example 20). In another example, the first binding site is a Ig domain 3 from VEGFR2 and the second binding site is Ig domain 2 from VEGFR2. In an example, the first and second binding domains are (in N- to C-terminal direction) the first and second VEGF binding sites of aflibercept (or Eylea™).

Suitable predetermined antibodies are ReoPro™; Abciximab; Rituxanh™; Rituximab; Zenapaxh™; Daclizumab; Simulecth™; Basiliximab; Synagis™; Palivizumab; Remicadeh™; Infliximab; Herceptinh™; Trastuzumab; Mylotargh™; Gemtuzumab; Campathh™; Alemtuzumab; Zevalinh™; Ibritumomab; Humirah™; Adalimumab; Xolair™; Omalizumab; Bexxarh™; Tositumomab; Raptivah™; Efalizumab; Erbituxh™; Cetuximab; Avastinh™; Bevacizumab; Tysabrih™; Natalizumab; Actemrah™; Tocilizumab; Vectibixh™; Panitumumab; Lucentish™; Ranibizumab; Solirish™; Eculizumab; Cimziah™; Certolizumab; Simponih™; Golimumab, Ilaris™; Canakinumab; Stelara™; Ustekinumab; Arzerrah™; Ofatumumab; Prolie™; Denosumab; Numaxh™; Motavizumab; ABThraxh™; Raxibacumab; Benlystah™; Belimumab; Yervoyh™; Ipilimumab; Adcetrish™; Brentuximab; Vedotin™; Perjeta™; Pertuzumab; Kadcyla™; Ado-trastuzumab; Gazyvam and Obinutuzumab. Also disclosed are the generic versions of these and the corresponding INN names—each of which is a suitable predetermined antibody for use as a source of antigen binding sites for use in the present invention. Suitable sequences of VH and VL domains of predetermined antibodies are disclosed in Table 4. Thus, for example, the multimer of the invention comprises a plurality (eg, 4, 8, 12, 16 or 20) copies of the VH/VL antigen binding site of any of these antibodies, eg, wherein the VH of the binding site is comprised by a polypeptide of the invention that comprises a SAM (eg, a TD) and each polypeptide is paired with a further polypeptide comprising the VL that pairs with the VH, thus forming an antigen binding site. In an example, the polypeptide comprising the SAM also comprises a CH1 which pairs with a CL of the further polypeptide. Optionally, the binding site of the polypeptide of the multimer comprises a VH of the binding site of the antibody and also the CH1 of the antibody (ie, in N- to C-terminal direction the VH-CH1 and SAM). In an embodiment, the polypeptide may be paired with a further polypeptide comprising (in N- to C-terminal direction a VL-CL, eg, wherein the CL is the CL of the antibody).

In one embodiment, the predetermined antibody is Avastin.

In one embodiment, the predetermined antibody is Actemra.

In one embodiment, the predetermined antibody is Erbitux.

In one embodiment, the predetermined antibody is Lucentis.

In one embodiment, the predetermined antibody is sarilumab.

In one embodiment, the predetermined antibody is dupilumab.

In one embodiment, the predetermined antibody is alirocumab.

In one embodiment, the predetermined antibody is evolocumab.

In one embodiment, the predetermined antibody is pembrolizumab.

In one embodiment, the predetermined antibody is nivolumab.

In one embodiment, the predetermined antibody is ipilimumab.

In one embodiment, the predetermined antibody is remicade.

In one embodiment, the predetermined antibody is golimumab.

In one embodiment, the predetermined antibody is ofatumumab.

In one embodiment, the predetermined antibody is Benlysta.

In one embodiment, the predetermined antibody is Campath.

In one embodiment, the predetermined antibody is rituximab.

In one embodiment, the predetermined antibody is Herceptin.

In one embodiment, the predetermined antibody is durvalumab.

In one embodiment, the predetermined antibody is daratumumab.

In another embodiment, the polypeptide comprises (in N- to C-terminal direction) a first antigen binding site, an optional linker (eg, a G₄Sₙ, wherein n=1, 2, 3, 4, 5, 6, 7, or 8, preferably 3), a second antigen binding site, a hinge sequence comprising a lower hinge and devoid of a core hinge region (and optionally devoid of an upper hinge region), an antibody Fc (eg, an IgG1 Fc) and a SAM (eg, a TD, such as a p53 TD). For example, the core hinge region sequence is a CXXC amino acid sequence. The polypeptide may comprise another antigen binding site (eg a dAb or scFv) between the Fc and SAM and/or C-terminal to the SAM. Optionally, the multimer comprises a plurality (eg, 4 copies) of such polypeptide. Optionally the binding sites have the same antigen specificity (eg, all bind TNF alpha). In another option, the first and second (and optionally said another binding site) bind to different antigens. The or each binding site can bind any antigen disclosed herein, eg, each binding site binds PD-L1, or the first binding site binds PD-L1 and the second binding site binds 41-BB, or the first binding site binds 4-1BB and the second binding site binds PD-L1 (as shown in Example 18).

The polypeptide, in one embodiment, comprises (in N- to C-terminal direction) a first antigen binding site (eg, a dAb), an optional linker (eg, a G₄Sₙ, wherein n=1, 2, 3, 4, 5, 6, 7, or 8, preferably 3), a second antigen binding site (eg, a dAb), an antibody CH1 (eg, human IgG1 CH1) and a SAM (eg, a TD, such as a p53 TD). The polypeptide may comprise another antigen binding site (eg a dAb or scFv) C-terminal to the SAM. Optionally, the multimer comprises a plurality (eg, 4 copies) of such polypeptide, for example wherein each polypeptide is paired with a further polypeptide comprising (in N- to C-terminal direction) a third antigen binding site (eg, a dAb), an optionally fourth antigen binding site (eg, a dAb), an antibody CL (eg, a human Cκ or Cλ) and optionally a further antigen binding site. For example, the fourth and further binding sites are omitted. In another example, the third and fourth binding sites, but not the further binding site, are present. In another example, the third and further (but not the fourth) binding sites are present. Optionally the binding sites have the same antigen specificity (eg, all bind TNF alpha). In another option, the first and second (and optionally said another said binding site) bind to different antigens. The or each binding site can bind any antigen disclosed herein, eg, each binding site binds TNF alpha (as shown in Examples 21 and 22). In an example, the first and third, or the second and third binding sites pair to form a VH/VL pair that is identical to the VH/VL binding site of an anti-TNF alpha antibody, such as adalimumab, golimumab, infliximab (or Humira™, Simponi™ or Remicade™). In an example, the first and third, or the second and third binding sites pair to form a VH/VL pair that is identical to the VH/VL binding site of an anti-PD-L1 antibody, such as avelumab (or Bavencio™) or atezolizumab (or Tecentriq™). In an example, the first and third, or the second and third binding sites pair to form a VH/VL pair that is identical to the VH/VL binding site of an anti-PD-1 antibody, such as nivolumab (or Opdivo™) or pembrolizumab (or Keytruda™). In an example, the first and third, or the second and third binding sites pair to form a VHNL pair that is identical to the VH/VL binding site of an anti-VEGF antibody, such as bevacizumab (or Avastin™) or ranibizumab (or Lucentis™). Predetermined antibodies as discussed above can be used as the source of the VH/VL pairs.

In an example, the polypeptide of the invention is any Quad polypeptide disclosed herein, eg, comprising the Quad amino acid shown in any of the Tables herein (eg, any one of SEQ ID Nos: 81-115, 151-162, 190, 191, 209-224 and 179) or encoded by any of the Quad nucleotide sequences in any of the Tables herein (eg, Table 9, 14 or 17), or having the structure of a polypeptide shown in Table 8. The SAM may be any SAM disclosed herein, eg, any p53 or homologue TD disclosed in any Table herein (eg, as shown in Table 7 or comprised by a protein in Table 13).

Where amino acid sequences are shown with plural histidines at their C-terminus (eg, “HHHHHH” optionally followed by “..AAA”), such histidines and the optional ..AAA are in one embodiment omitted and the corresponding nucleotides encoding this are omitted from the nucleic acid encoding the amino acid sequence. Where amino acid sequences are shown with a DYKDDDDK motif (eg, a DYKDDDDKHHHHHH or DYKDDDDKHHHHHH..AAA), such a motif is in one embodiment omitted and the corresponding nucleotides encoding this are omitted from the nucleic acid encoding the amino acid sequence.

Any example, configuration, Aspect, Concept, Clause or Paragraph or disclosure herein is combinable with any feature of any further example, configuration, Aspect, Concept, Clause or Paragraph or disclosure herein.

It will be understood that particular embodiments described herein are shown by way of illustration and not as limitations of the invention. The principal features of this invention can be employed in various embodiments without departing from the scope of the invention. Those skilled in the art will recognise, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications (including US equivalents of all mentioned patent applications and patents) are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### EXAMPLES

### Example 1: Generation of Tetravalent and Octavalent Soluble Heterodimeric NY-ESO-1 TCR Molecules

This example demonstrates a method for generating tetravalent and octavalent soluble heterodimeric TCR molecules referred to as ts-NY-ESO-1 TCR and os-NY-ESO-1 TCR respectively. These formats overcome the problems associated with solubility and avidity for cognate ligand at the target site.

To exemplify ts-NY-ESO-1 TCR and os-NY-ESO-1 TCR as stable and soluble molecules, TCR αβ variable sequences with high affinity for NY-ESO-1 together with immunoglobulin constant domains and the NHR2 tetramerisation domain are used in this example.

The high-affinity NY-ESO TCR αβ chains (composing of TCR Vα-Cα and Vβ-Cβ respectively) specific for SLLMWITQC-HLA-A*0201 used in this example is as reported in WO 2005/113595 A2 with the inclusion of a signal peptide sequence (MGWSCIILFLVATATGVHS). To aid protein purification, a histidine tag was incorporated to the C-terminus of NHR2 domain.

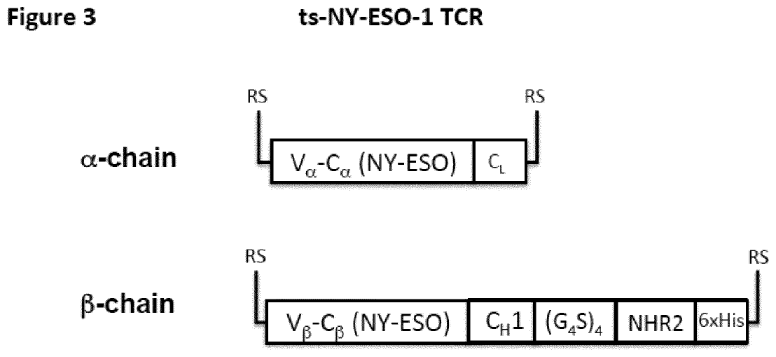

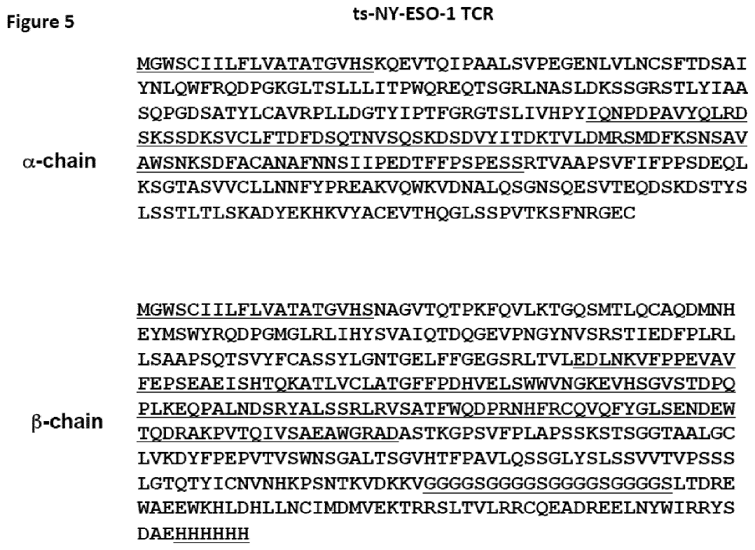

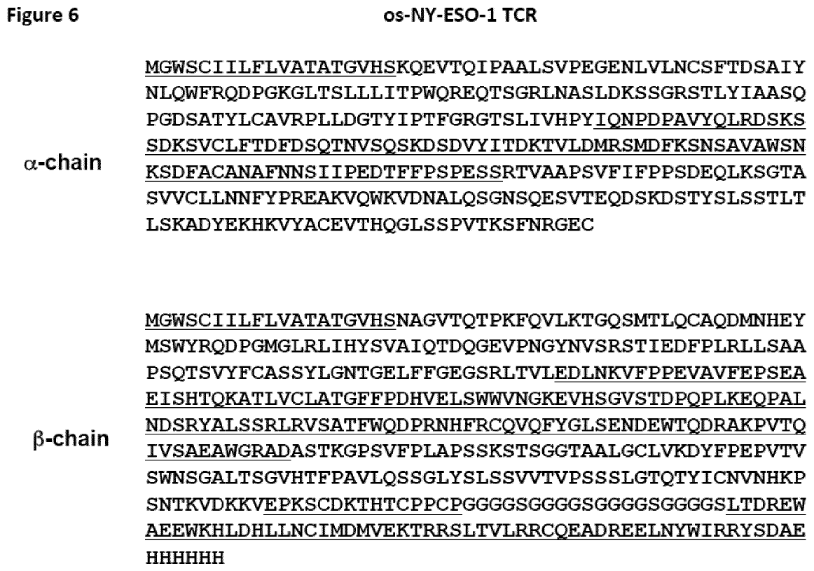

DNA constructs encoding components of ts-NY-ESO-1 TCR and os-NY-ESO-1 TCR are synthetically constructed as a two-vector system to allow for their soluble expression and functional assembly in mammalian cells. A schematic representation of the two assembled TCR chains (a and p chains) whose DNA sequences are synthesized for cloning into the expression vector are shown in FIGS. 3 and 4 and their amino acid sequences are shown in FIGS. 5 and 6.

The pTT5 vector system allows for high-level transient production of recombinant proteins in suspension-adapted HEK293 EBNA cells (Zhang et al., 2009). It contains origin of replication (oriP) that is recognized by the viral protein Epstein-Barr Nuclear Antigen 1 (EBNA-1), which together with the host cell replication factor mediates episomal replication of the DNA plasmid allowing enhanced expression of recombinant protein. Therefore the pTT5 expression vector is selected for cloning the components for the ts-NY-ESO-1 TCR and os-NY-ESO-1 TCR molecules.

Synthesized DNA fragments containing the TCR αβ chains are digested with restriction enzymes at the restriction sites (RS) (FastDigest, Fermantas) and the DNA separated out on a 1% agarose gel. The correct size DNA fragments is excised and the DNA purified using Qiagen gel extraction kit. The pTT5 vector was also digested with the same restriction enzymes and the linearized plasmid DNA is purified from excised agarose gel. The digested TCR αβ chains is cloned into the digested pTT5 vector to give four expression vectors (pTT5-ts-NY-ESO-1-TCRα, pTT5-ts-ESO-1-TCRs, pTT5-os-NY-ESO-1-TCRα and pTT5-os-ESO-1-TCRs).

Expression of Tetravalent and Octavalent Soluble NY-ESO TCR

Functional expression of ts-NY-ESO-1 TCR and os-NY-ESO-1 TCR is carried out in suspension-adapted HEK293 EBNA cells. HEK293-EBNA cells are cultured in serum-free Dulbecco's Modified Eagle Medium (DMEM, high glucose (4.5 g/L) with 2 mM L-glutamine) at 37° C., 5% CO₂ and 95% humidity.

For each transfection, HEK293-EBNA cells (3×10⁷ cells) are freshly seeded into 250 mL Erlenmeyer shaker Flask (Corning) from ˜60% confluent cells. Transfections are carried out using FreeStyle MAX cationic lipid base reagent (Life Technologies) according to the supplier's guidelines. For expression of ts-NY-ESO-1 TCR, 37.5 μg of total plasmid DNA (18.75 μg plasmid DNA each of pTT5-ts-NY-ESO-1-TCRα and pTT5-ts-ESO-1-TCRβ vectors are used or varying amounts of the two expression plasmids) are used per transfection. Similarly for expression of os-NY-ESO-1 TCR, 18.75 μg plasmid DNA each of pTT5-os-NY-ESO-1-TCRα and pTT5-os-ESO-1-TCRβ is are used for transfection. Following transfection, cells were recovered in fresh medium and cultivated at 37° C. with 5% CO₂ in an orbital shaker at 110 rpm for between 4-8 days. Smaller scale transfections are done similarly in 6 well or 24 well plates.

Analysis of Expressed Soluble eTCR²-BiTE

The ts-NY-ESO-1 TCR and os-NY-ESO-1 TCR protein molecules secreted into the supernatant are analyzed either directly by sodium dodecyl sulfate (SDS) polyacrylamide gel electrophoresis (PAGE) or after protein purification. Protein samples and standards are prepared under both reducing and non-reducing conditions. SDS-PAGE was performed using cast mini gels for protein electrophoresis in a Mini-PROTEAN Tetra cell electrophoresis system (Bio-Rad). Coomassie blue dye was used to stain proteins in SDS-PAGE gel.

### Purification of Ts-NY-ESO-1 TCR and Os-NY-ESO-1 TCR Protein Molecules

Soluble ts-NY-ESO-1 TCR and os-NY-ESO-1 TCR from cell supernatant are purified in two steps. In the first step immobilized metal affinity chromatography (IMAC) are used with nitrilotriacetic acid (NTA) agarose resin loaded with nickel (HisPur Ni-NTA Superflow agarose—Thermo fisher). The binding and washing buffer consists of Tris-buffer saline (TBS) at pH7.2 containing low concentration of imidazole (10-25 mM). Elution and recovery of the His-tagged ts-NY-ESO-1 TCR and os-NY-ESO-1 TCR from the IMAC column are achieved by washing with high concentration of imidazole (>200 mM). The eluted protein fractions are analysed by SDS-PAGE and the fractions containing the protein of interest are pooled. The pooled protein fraction is used directly in binding assays or further purified in a second step involving size-exclusion chromatography (SEC). Superdex 200 increase prepacked column (Gelifesciences) are used to separate out monomer, oligomer and any aggregated forms of the target protein.

### Surface Plasmon Resonance

The specific binding and affinity analysis of ts-NY-ESO-1 TCR and os-NY-ESO-1 TCR to its pMHC is performed using BIAcore. Briefly, the purified Histidine-tagged ts-NY-ESO-1 TCR and os-NY-ESO-1 TCR proteins are captured onto sensor surface via Ni²⁺ chelation of nitrilotriacetic acid (NTA). Varying concentration of the analyte solution containing NY-ESO pep_{(SLLMWITQV)}-MHC (ProImmune) is injected and the binding signals were monitored

### Example 2: Generation of Tetravalent and Octavalent Soluble NY-ESO TCR-IL2 Fusion Molecule

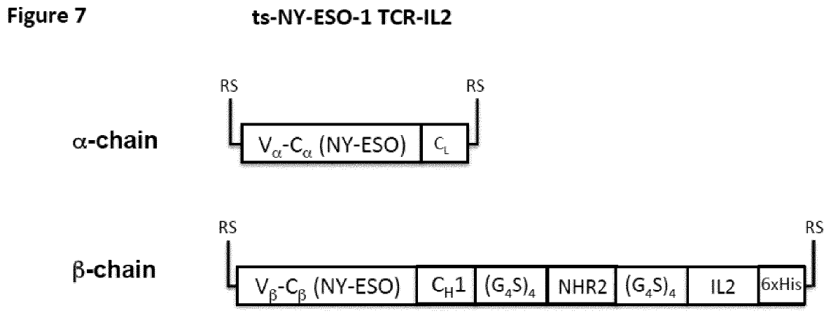

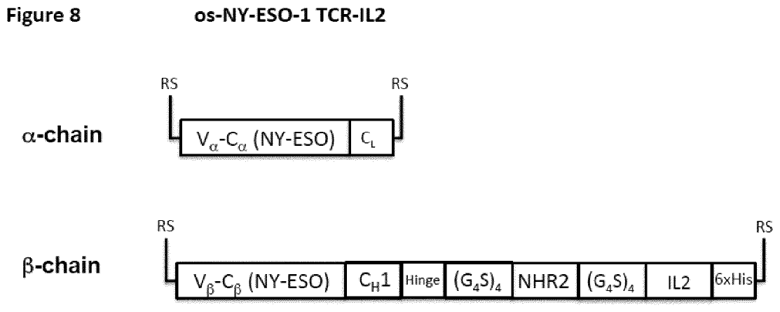

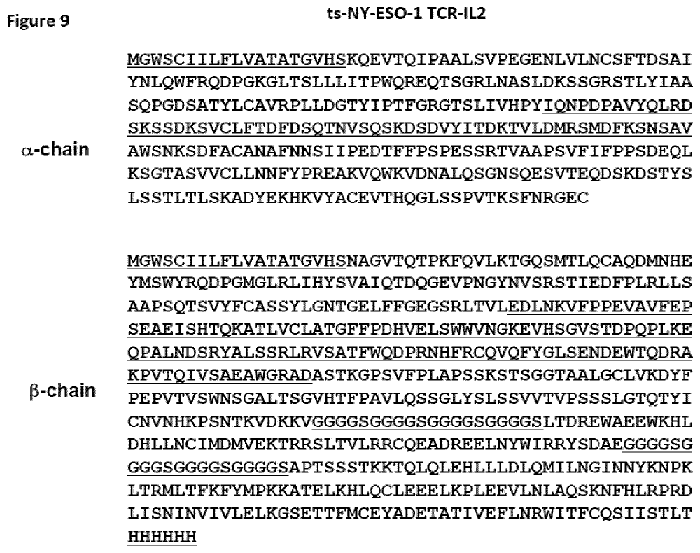

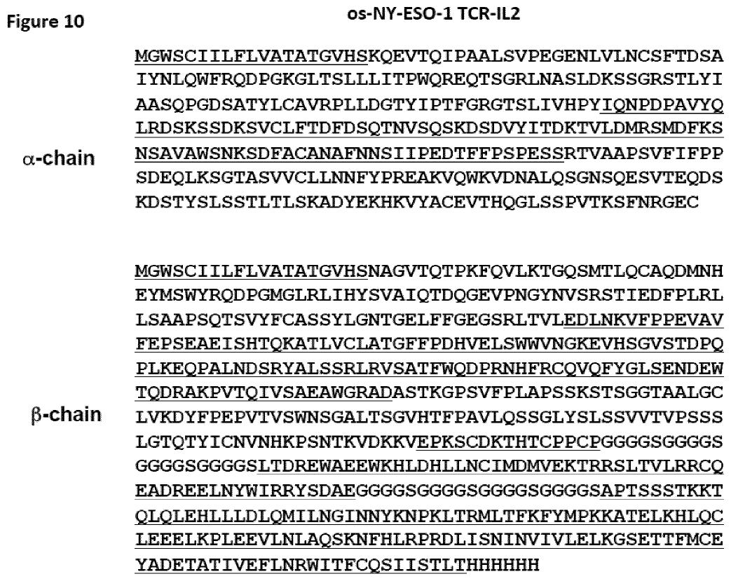

The DNA encoding the domains required for expressing ts-NY-ESO-1 TCR-IL2 and os-NY-ESO-1 TCR-IL2 protein complexes are synthesized and cloned into the expression vector pTT5 as described above. A schematic representation of the domains within the TCR αβ chains for ts-NY-ESO-1 TCR-IL2 and os-NY-ESO-1 TCR-IL2 are shown in FIGS. 7 and 8 and the amino acids sequences are shown in FIGS. 9 and 10.

Expression, purification and characterization of ts-NY-ESO-1 TCR-IL2 and os-NY-ESO-1 TCR-IL2 are carried out as described above.

### Example 3: High Yield Tetramer Secretion

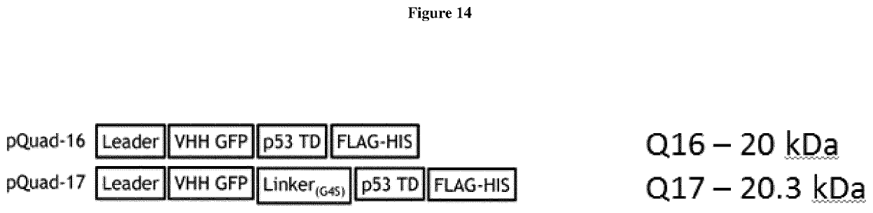

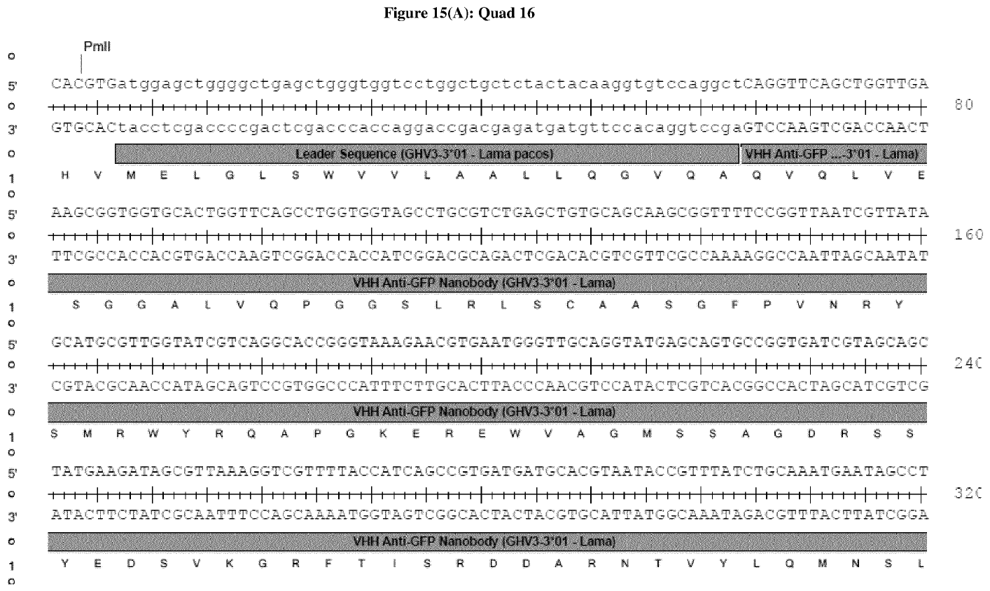

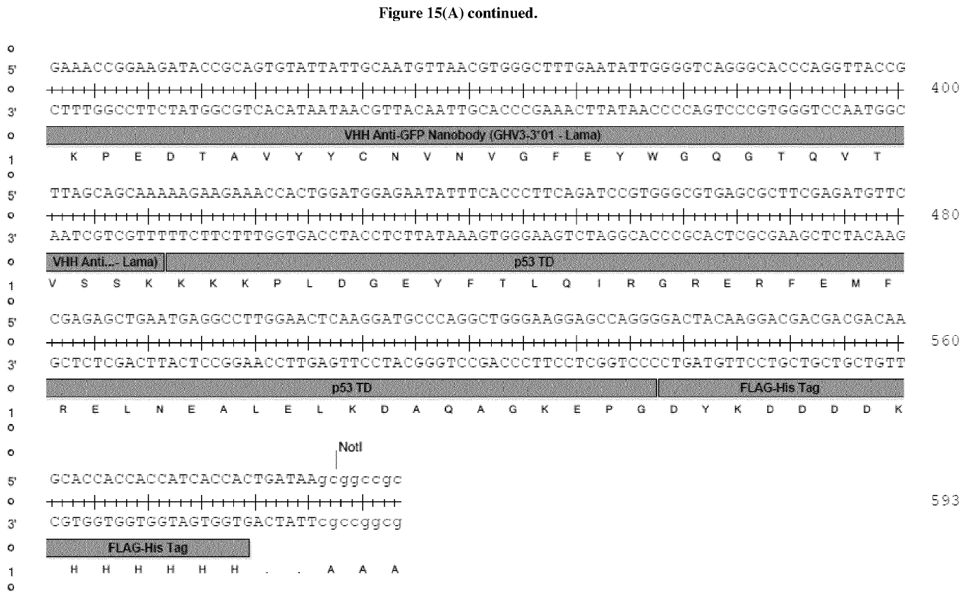

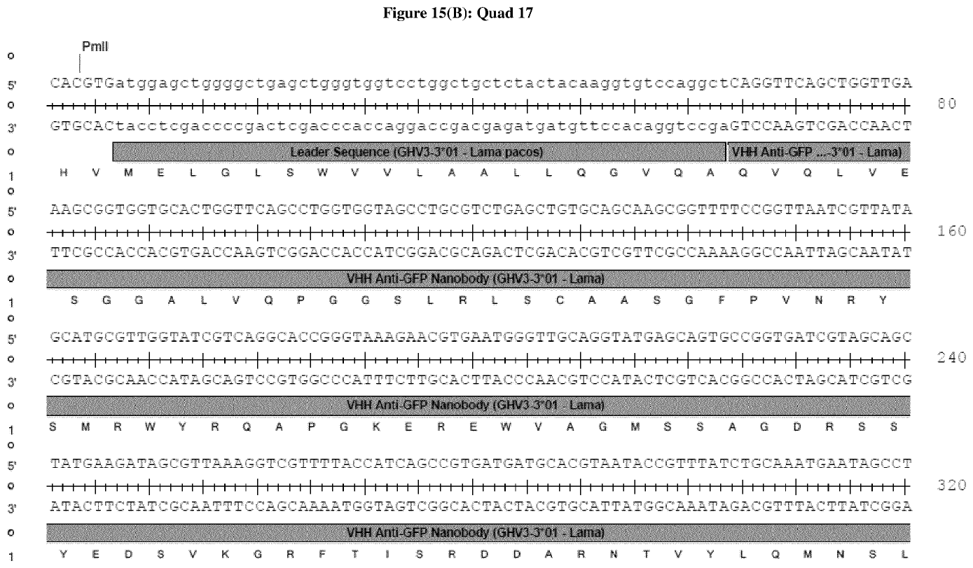

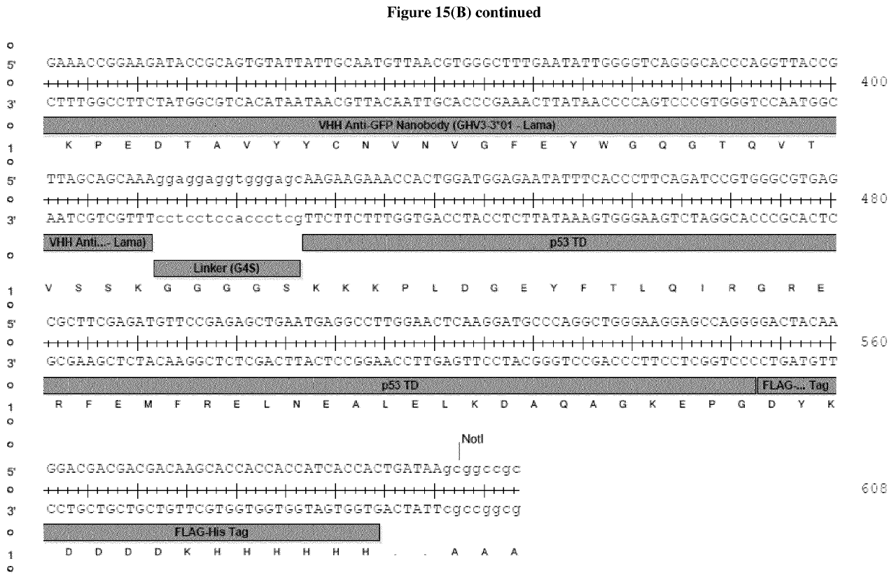

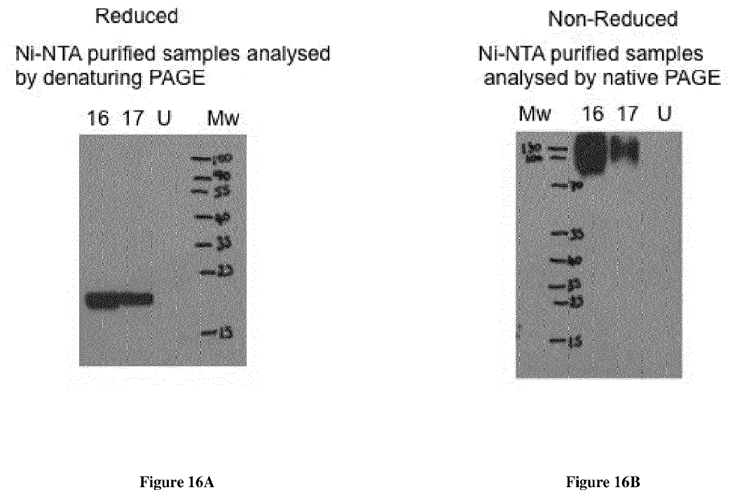

Briefly, using conventional genetic engineering techniques, a HEK293-T cell line was made that encodes Quad 16 (FIGS. 14 and 15) and another HEK293-T cell line was made that encodes Quad 17 (FIGS. 14 and 15).

Protein expression took place and protein was secreted from the cell lines. Samples of the medium in which the cells were incubated were subjected to PAGE under denaturing conditions (SDS-PAGE) or under native conditions (no SDS). The former was further under reduced conditions (using mercaptoethanol), whereas the latter was not.

The reduced gel showed a distinct banding (FIG. 16) at the expected monomer size. Surprisingly, the unreduced, native gel showed no detectable banding at the monomer, dimer or trimer size, but instead heavy banding was seen at the tetramer size indicating that a very high yield of tetramer had been obtained, and this was confirmed by SEC to be of high purity.

For Quad 16, the tetramer peak from SEC was run on SDS-PAGE and the obtained band was cut out for mass spectrometry. The data were obtained with trypsin digests and p53 was detected in 100% of the protein. This was conclusive evidence that the secreted Quad 16 was multimerised

### Example 4: Intracellular Protein Expression of Extracellular Portion of TCR Fused to NHR2 TD

### Expression Vector

All DNA fragments were synthesized and cloned into the expression vector, pEF/myc/cyto (Invitrogen) by Twist Bioscience (California). Schematics and sequences of the synthesized DNA fragments and Quad polypeptides are shown in FIG. 21, and the sequence tables herein.

### DNA Preparation

Lyophilised plasmid DNA synthesized by Twist Bioscience, were resuspended with MQ water to a concentration of 50 ng/μl. 50 ng of DNA was transformed into 50 μl of competent DH5α cells using a conventional heat shock method. The cells were plated on LB agar plates containing 100 μg/mL ampicillin and grown overnight at 37° C. Individual colonies were picked and grown overnight at 37° C., 220 rpm. The DNA was purified from the cells using the QIAprep Spin Miniprep Kit, according to the manufacturers instructions (Qiagen).

### Transfections in HEK293T Cell

Briefly, HEK293T cells were maintained in high glucose DMEM supplemented with 10% FBS and Pen/Strep. Cells were seeded at 6×10⁵ cells per well of a 6-well plate in 2 ml media and were allowed to adhere overnight at 37° C., 5% CO₂. 7.5 μl of Lipofectamine 2000 was diluted in 150 μl of OptiMem and incubated at room temperature for 5 mins. Plasmid DNA (2.5 μg) was diluted in 150 μl of OptiMem. Diluted DNA was combined with the diluted Lipofectamine 2000, mixed gently and incubated at R.T. for 20 mins. The 300 μl of complexes were added to one well of the 6-well plates. When analysis required the media to be serum free, the media was aspirated and replaced with CD293 media 6 hours post-transfection. The cells were incubated for 48 hours at 37° C., 5% CO₂ prior to analysis.

Accordingly, different formats of TCR-linked NHR2 tetramerisation domain (TD) constructs (Quads) were transfected into HEK293T cells. Quads 3 & 4 resembling a TCR tetravalent format (structure schematically represented in FIGS. 1 & 3) and Quads 12 & 13 resembling a TCR octavalent format (Structure schematically represented in FIGS. 2 & 4) were transfected for protein expression analysis. Protein samples were prepared from transfected HEK293T cells as follows to check for intracellularly expressed protein. Briefly cells were washed once with 2 ml PBS, which was subsequently aspirated. 150 μl of Trypsin-EDTA (0.05%) was added to each well and the cells were incubated at R.T. for ˜1 min. The plate was tapped to lift any strongly adhering cells. 850 μl of media was added to each well to inactivate the trypsin. The cells were transferred to a 1.5 ml eppendorf and spun at 1,000 rpm for 5 mins. The supernatant was aspirated and the pellets stored on ice. The cells were resuspended in 400 μl cell lysis buffer (10 mM Tris pH 7.5, 1% SDS) containing Protease Inhibitor Cocktail Set III (Calbiochem), diluted 1/200. The samples were vortexed vigorously and incubated on ice for 20 mins. The cells were sonicated using a Branson Ultrasonics Sonifier™ (Thermo Fisher Scientific). The amplitude was set to 30% and the cells were sonicated for a total of 24 seconds (6 secs on, 3 secs off ×4). The total protein concentration was quantified using the Pierce BCA Protein Assay Kit™, according to the manufacturer's instructions. 100 μg was diluted with MQ water to give a volume of 80 μl. 20 μl of 5×SDS loading buffer was then added giving samples of 1 mg/ml. Samples were incubated at 95° C. for 5 min prior to SDS-PAGE and Western blot analysis.

Protein samples were separated on SDS-PAGE under denaturing condition. Typically, 25 μg of whole cell lysate (25 μl) were loaded on to the gel for Western blot analysis. 5 μl of PageRule Prestained 10-180 kDa Protein Ladder was loaded into the gel alongside the protein samples. The gels were run in Tris-Glycine buffer containing 0.1% SDS. A constant voltage of 150 volts was used and the gels were run for ˜70 mins until the dye front has migrated fμlly.

SDS-PAGE (15% Bis-Tris) gels were prepared using the following resolving and stacking gels.

Resolving Gel:
5 ml 30% Bis-Acrylamide
2.6 ml 1.5 M Tris (pH 8.8)
50 μl 20% SDS
100 μl 10% APS
10 μl TEMED
2.2 ml MQ Water

Stacking Gel:
0.75 ml 30% Bis-Acrylamide
1.25 ml 1.5 M Tris (pH 8.8)
25 μl 20% SDS
50 μl 10% APS
5 μl TEMED
2.9 ml MQ Water

Western blotting was performed for the specific and sensitive detection of protein expression of TCR-NHR2 TD fusion proteins from Quads 3, 4, 12 and 13. Proteins separated out on SDS-PAGE were transferred onto Amersham Hybond™ 0.45 μM PVDF membrane as follows. Briefly, Amersham Hybond 0.45 μM PVDF membrane was activated with MeOH for ˜1 min and rinsed with transfer buffer (25 mM Tris, 190 mM Glycine, 20% MeOH) before use. The sponge, filter paper, gel, membrane, filter paper, sponge stack was prepared and placed in the cassette for transfer. Transfer was carried out on ice at 280 mA for 75 mins. The membrane was incubated for ˜2 hours in blocking buffer (TBST, 5% milk powder). The membrane was washed briefly with TBST before being incubated at 4° C. overnight with anti-human IgG HRP (Thermo, 31410) diluted 1/2500 in TBST, 1% milk powder. The membrane was washed thoroughly (three washes of TBST, 15 mins each) before being developed using the Pierce ECL Western Blotting Substrate.

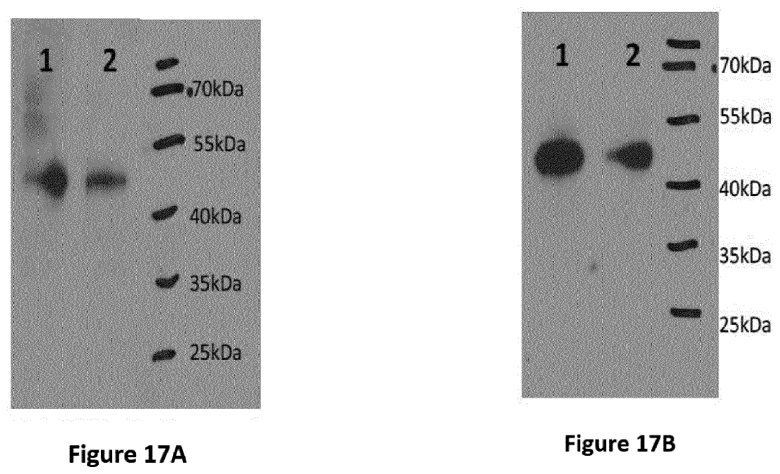

Using anti-human IgG detection antibody to probe Western blots, specific protein band at the expected molecular weight can be detected from samples prepared from Quads 3 (46.1 kDa), 4 (46.4 kDa), 12 (47.8 kDa) and 13 (48.1 kDa) (FIGS. 17A and 17B). These data confirm intracellular protein expression of TCR-NHR2 TD fusion proteins in HEK293T cells.

For all of the Quads analysed, a clear single band can be detected indicating TRVβ-TRCβ-IgG1-CH1 (+/−IgG hinge domain) fusions with the NHR2 TD are stable. These expression data also confirm the possibility of assembling tetravalent (Quads 3 & 4) and octavalent (Quads 12 & 13) molecules as exemplified in this example.

The difference between Quads 3 and 4 is the presence of a small peptide linker (G4S) located between the IgG1 CH1 domain and NHR2 TD. This is also true for Quads 12 and 13 where Q13 contains a peptide linker between the IgG1 CH1 domain and NHR2 TD. From the expression data, it can be seen the peptide linker does not effect protein expression. However, it may be desirable to include a peptide linker to aid antigen binding and or stabilizing the multimerisation complex in these TCR-NHR2 TD formats.

### Example 5: Soluble Protein Expression of Extracellular Portion of TCR Fused to NHR2 TD

TCR-NHR2 TD fusion proteins were shown in Example 4 to be expressed intracellularly in HEK293T cells. Here again Quads 3, 4, 12 and 13 were used to demonstrate soluble expression of these fusion proteins. As described above, Quads 3, 4, 12 and 13 were transfected into HEK293T cells and soluble proteins from the cell supernatant were concentrated. Briefly, the media was harvested 48 hours post-transfection and centrifuged at 2,000 rpm for 5 mins to remove any cells or debris. Typically, 500 μl of media was concentrated to 100 μl using Amicon™ Ultra 0.5 Centrifugal Units with a MWCO of 10 kDa. 25 μl of 5×SDS loading buffer was added to the sample, which was then incubated at 95° C. for 5 mins prior to gel/Western blot analysis. Concentrated protein samples were separated out on SDS-PAGE gel and transferred onto Amersham Hybond 0.45 μM PVDF membrane. Western blotting and protein detection was done using anti-human IgG HRP using the methods described above.

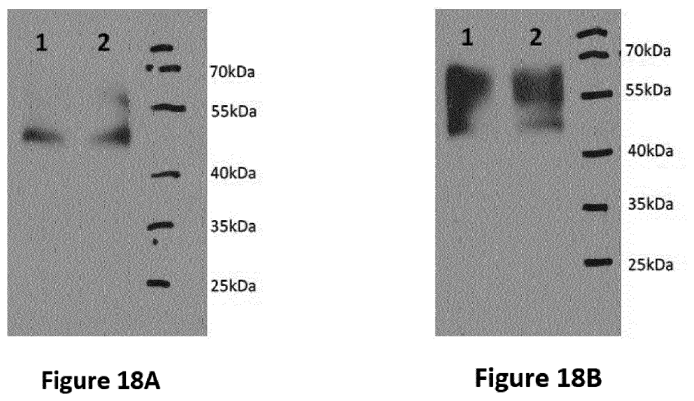

Protein samples concentrated and prepared from cell supernatants show specific protein band at the expected molecular weight on Western blots corresponding to Quads 3 (46.1 kDa), 4 (46.4 kDa), 12 (47.8 kDa) and 13 (48.1 kDa) (FIGS. 18A and 18B). The Western blot expression data unequivocally shows soluble expression of TCR-NHR2 TD fusion proteins in HEK293T. These data are the first report demonstrating soluble expression of TCR-NHR2 TD fusion proteins expressed in eukaryotic cells such as HEK293T cells.

Detection of soluble protein expression from both tetrameric (Quads 3 & 4) and octameric (Quads 12 & 13) TCR-NHR2 TD formats highlights the potential applicability of NHR2 TD in a broad setting. Use of NHR2 TD fusion molecules could be used for the preparation of therapeutic molecules and protein molecules for use in diagnostics and imaging.

### Example 6: Intracellular Protein Expression of Antibody Fragments Fused to NHR2 TD

To further exemplify the versatility of NHR2 TD, several different antibody fragment formats fused to NHR2 TD were constructed for testing their expression in HEK293T cells.

Quads 14 and 15 contain an antibody VH domain fused to NHR2 TD either with or without a peptide linker located between the VH and NHR2 TD as schematically depicted in FIGS. 11 and 21. The VH domain in Quads 14 and 15 are specific for GFP (green fluorescent protein). Several other versions of this format were also constructed and tested with VH specific for therapeutically useful drug targets. Sequences of the binding domains are listed in Table 4. Some of these include Quad 34 (specific for TNFα), Quad 38 (specific for VEGF), Quad 40 (specific for EGFR) and Quad 44 (specific for CD38).

Quads 38 and 44 were further developed to include an additional binding arm with the inclusion of a second VH domain specific for EGFR and CD138 respectively yielding Quads 42 and 46. Quads 42 and 46 represent bispecific molecules with the capability to multimerise via the NHR2 TD domain to form bispecific tetramers.

In another example, an effector molecule (human IL2) was linked to the C-terminus of Quads 14 & 15 resulting in Quads 18 and 19, whereby the VH-NHR2-IL2 molecule is tetravalent and bifunctional.

In another example, antibody Fab fragment (VH-CH1) was linked to NHR2 TD (Quads 23 and 24) and as schematically depicted in FIG. 12. Quads 23 and 24 represent tetravalent Fab molecules when co-expressed or mixed and assembled in-vitro with a second chain containing immunoglobulin light chain (e.g. Quad 25).

In yet another example, a human IgG1 hinge domain was included to Quads 23 and 24, which is referred to as Quads 26 and 27 and as schematically depicted in FIG. 13. Quads 26 and 27 represent octavalent Fab molecules when co-expressed or mixed and assembled in-vitro with a second chain containing immunoglobulin light chain domains (e.g. Quad 25).

The following Quad vectors, Quads 14, 15, 18, 19, 23, 24, 26, 27, 34, 38, 40, 42, 44 and 46 all of which are His-tagged were transfected in HEK293T cells. Protein samples were prepared from whole-cell extracts as described above, separated out on SDS-PAGE and transferred onto Amersham Hybond 0.45 μM PVDF membrane. Specific protein expression were probed using anti-His HRP (Sigma, A7058) diluted 1/2500 in TBST, 1% milk powder.

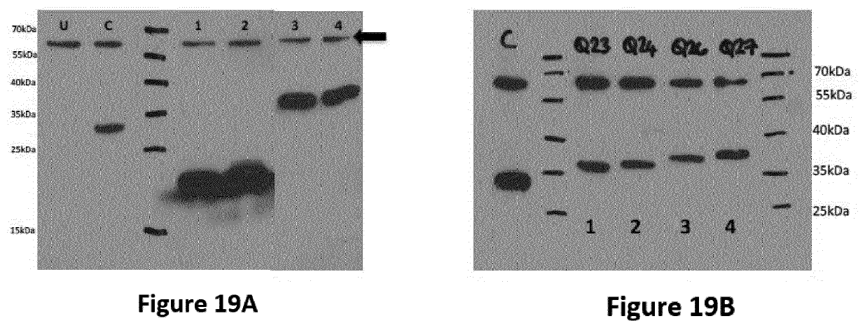

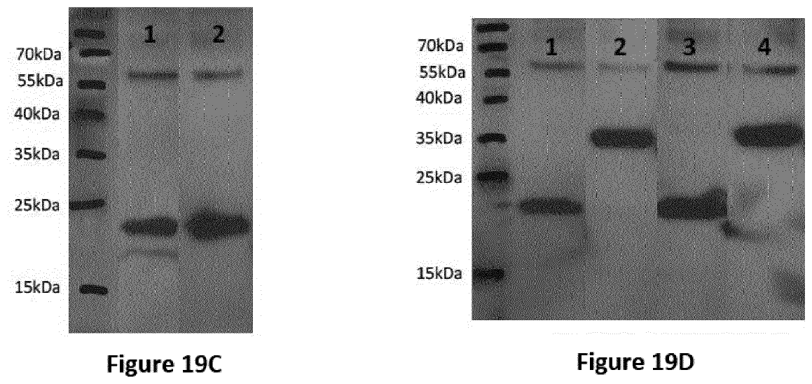

Specific protein expression in whole cell extracts could be detected for all the different antibody-NHR2 TD fusion proteins tested using Quads 14, 15, 18, 19, 23, 24, 26, 27, 34, 38, 40, 42, 44 and 46 (FIGS. 19A-D). Interestingly, Quads 18 and 19 containing an effector domain (IL2) fused to the C-terminus of NHR2 TD domain, in addition to the VH binding domain fused to the N-terminus of NHR2 TD showed good protein expression.

Expression of Quads 23 and 24 polypeptides highlights the potential to use NHR2 TD to form tetravalent antibody Fab molecules when co-expressed or mixed in-vitro with a partner soluble Quad molecule (e.g. Quad 25). Similarly expression of Quads 26 and 27, which include human IgG1 hinge domain highlight the potential to use NHR2 TD to form octavalent antibody Fab molecules when co-expressed or mixed in-vitro with a partner soluble second partner chain (e.g. Quad 25).

Quads 42 and 46 bispecific molecules containing an additional VH domain fused to the C-terminus of NHR2 TD domain also showed good protein expression. These data highlights the versatility of the NHR2 TD domain and its ability to be fused to different binding and effector molecules for developing a vast array of protein formats. The data also suggest it is possible to fuse protein molecules to both the N-terminus and C-terminus of NHR2 TD, which allows for the development of bispecific multivalent protein molecules.

### Example 7: Multivalent Assembly of Antibody Fragments Fused to NHR2 TD

NHR2 TD is responsible for the oligomerisation of ETO into a tetrameric complex. Using the NHR2 TD domain, it is possible to fuse binding domains and effector molecules to the N-terminus or C-terminus or both N- and C-terminus without effecting expression as shown in examples 4-6. Binding domains could be TCR variable and constant domains, antibody and antibody fragments or effector molecules such as IL2. It is also possible to express proteins in a soluble format when fused to NHR2 TD (FIG. 18) despite NHR2 TD being a part of an intracellularly expressed protein where in nature it is only expressed inside the cell.

To demonstrate whether NHR2 TD retains its potential to oligomerise once it is fused to a binding domain, Quads 14 and 15 were expressed in HEK293T cells and protein samples were prepared from whole cell extracts as described above. Protein samples were separated out on PAGE gel under denaturing and non-denaturing (native) conditions. Native gels were prepared using the protocol described above, but without SDS. Proteins from PAGE gels were transferred onto Amersham Hybond 0.45 μM PVDF membrane. Specific protein expression was probed with anti-human IgG HRP detection antibody.

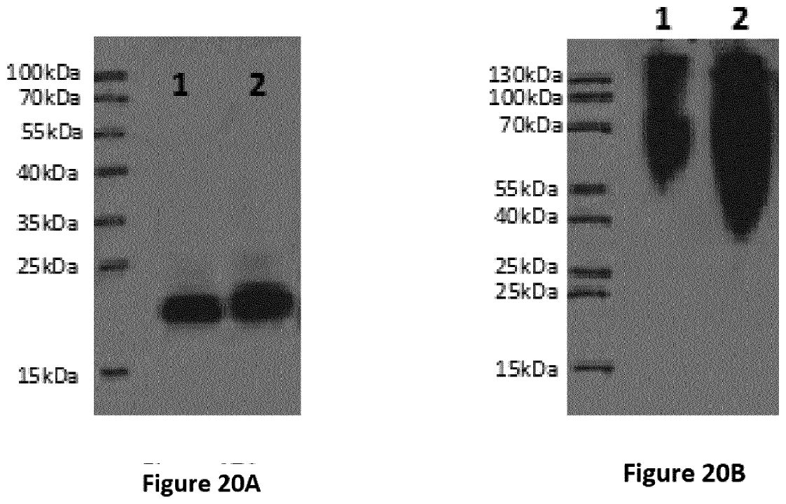

As expected under denaturing conditions, expression of VH-NHR2 TD from Quads 14 and 15 can be seen as a monomer where a specific protein band can be detected at the expected molecular weight (22 and 22.3 kDa) (FIG. 20A). Under non-denaturing and thus native conditions, interestingly no monomer or dimer of VH-NHR2 TD from Quads 14 and 15 can be detected (FIG. 20B). Only a high molecular weight protein band believed to be tetramers of VH-NHR2 TD from Quads 14 and 15 can be detected. The assembly of tetramers appears to be highly efficient and pure judging by the protein intensity and the absence of any detectable monomers and dimers of Quads 14 and 15.

Together with the data in examples 4-7, there is conclusive evidence NHR2 TD is highly versatile allowing fusion of various protein binding domains and effector molecules. NHR2 TD allows soluble expression of proteins from eukaryotic cells such as HEK293T cells and they form highly stable and pure tetrameric molecules.

### FURTHER EXAMPLES

### Methods

Expression Vectors

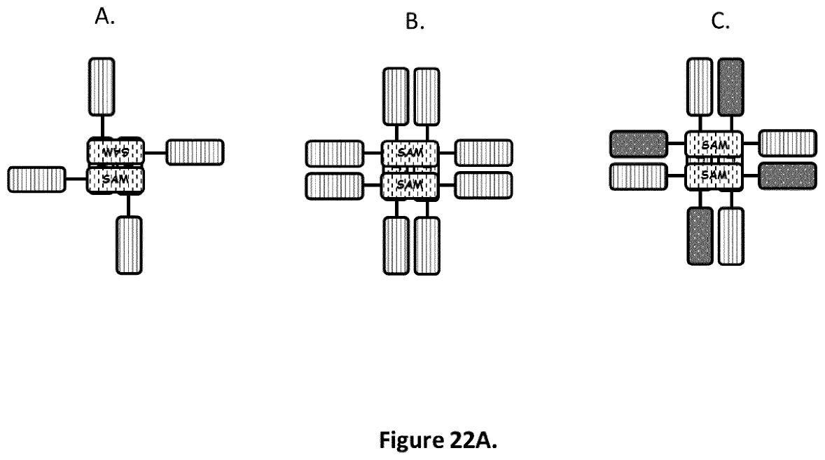

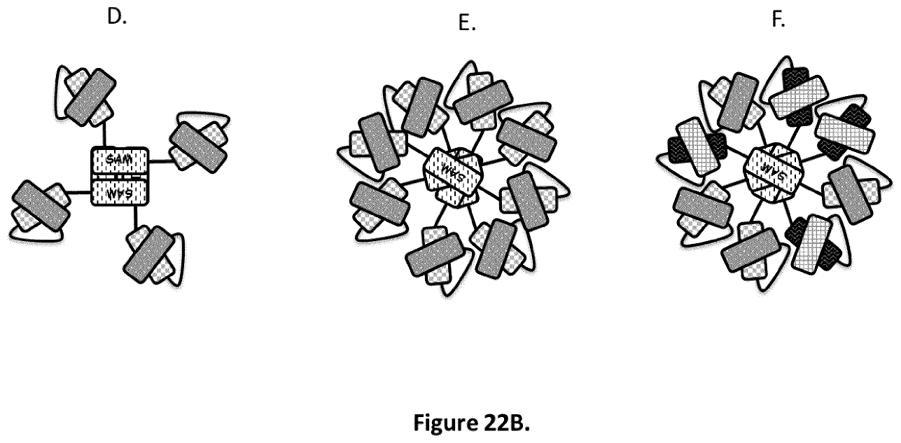

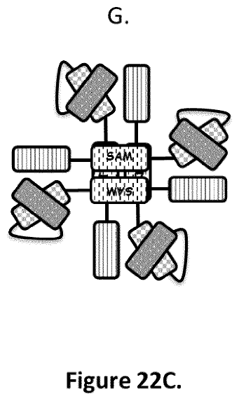

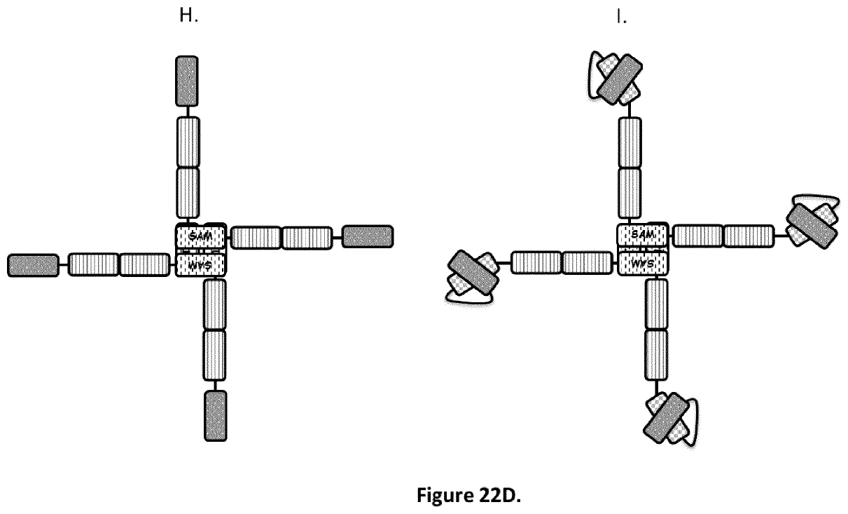

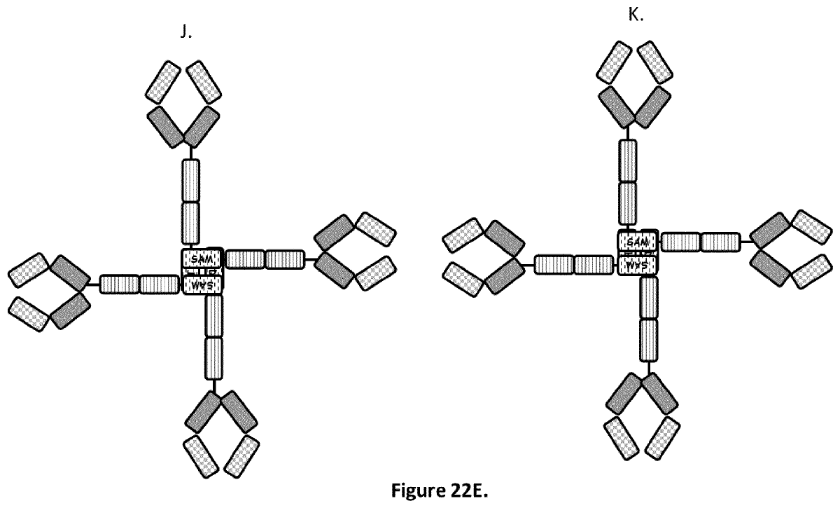

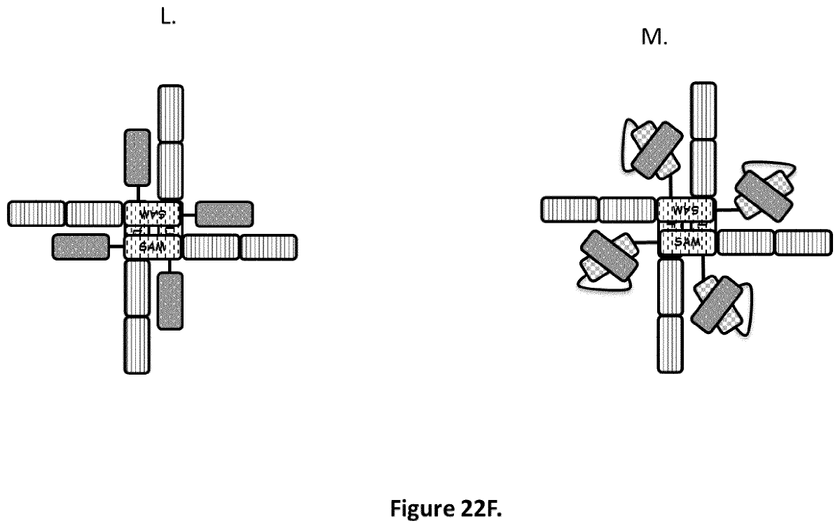

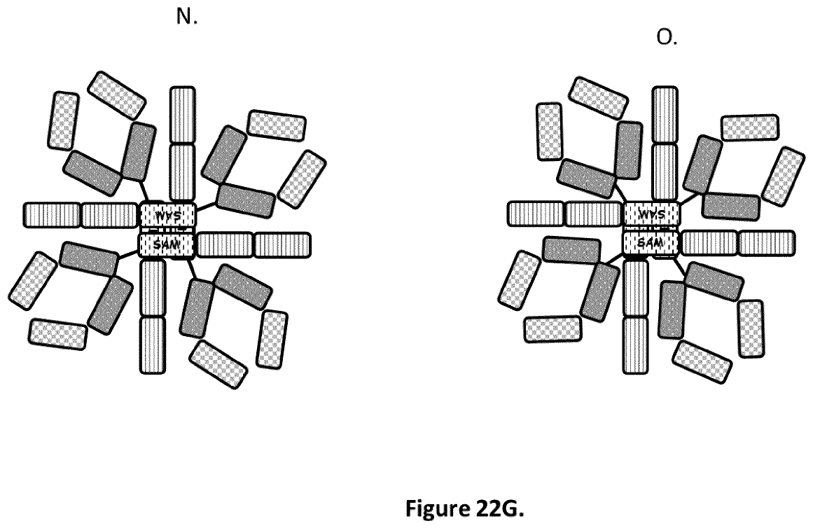

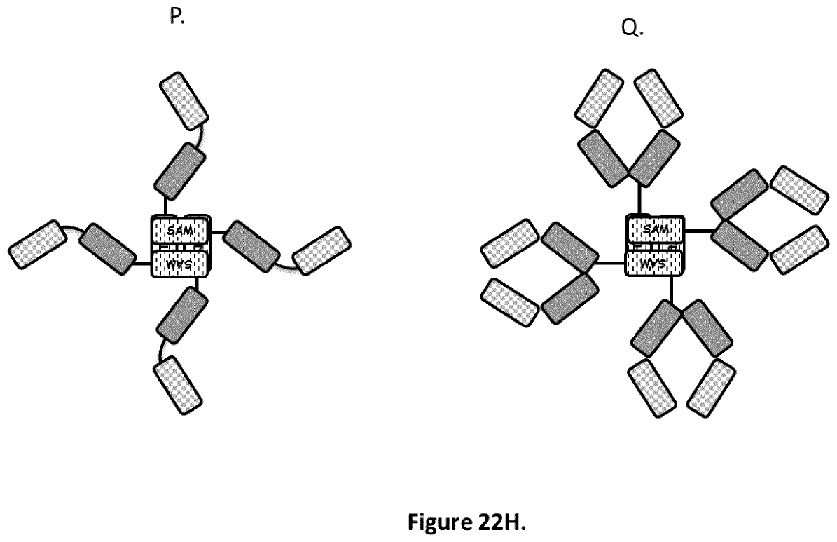

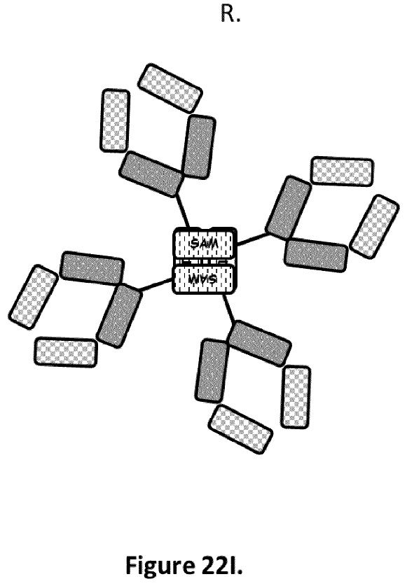

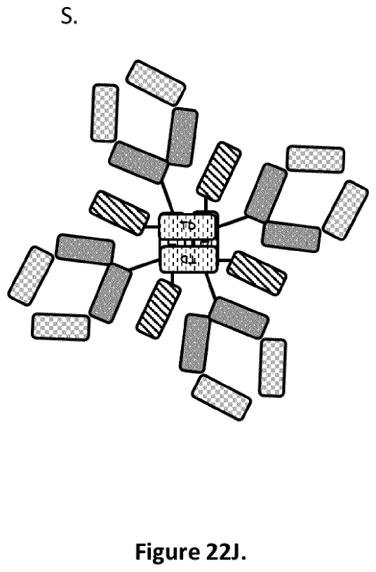

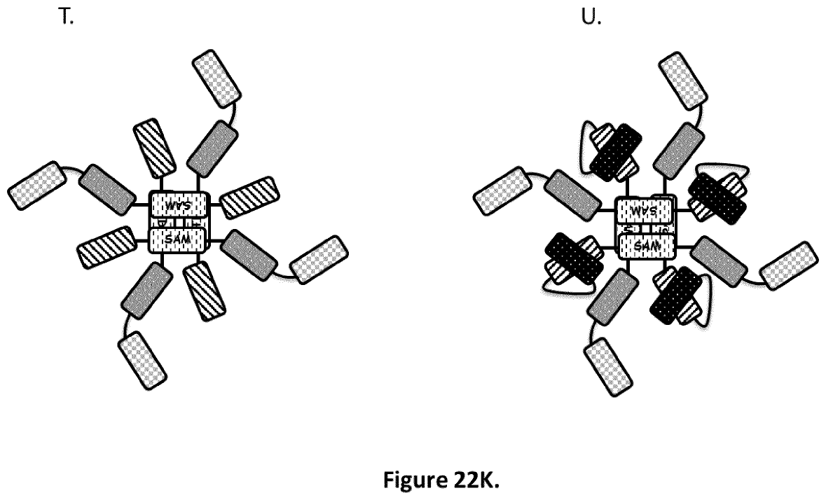

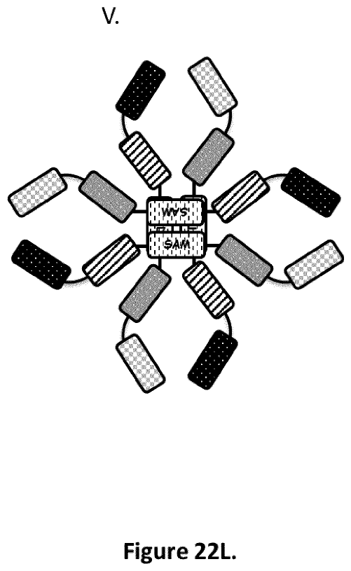

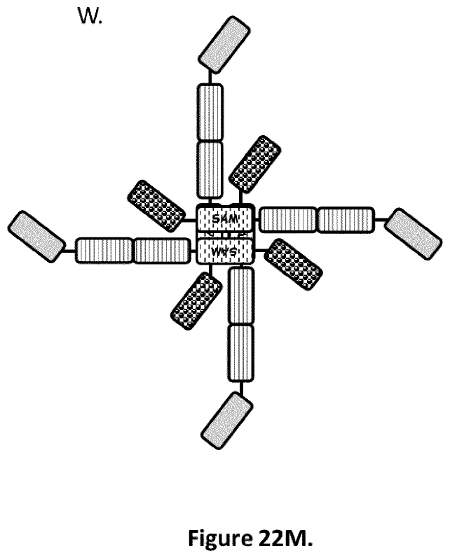

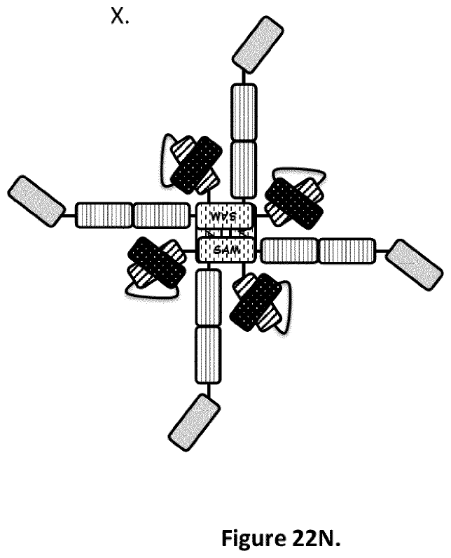

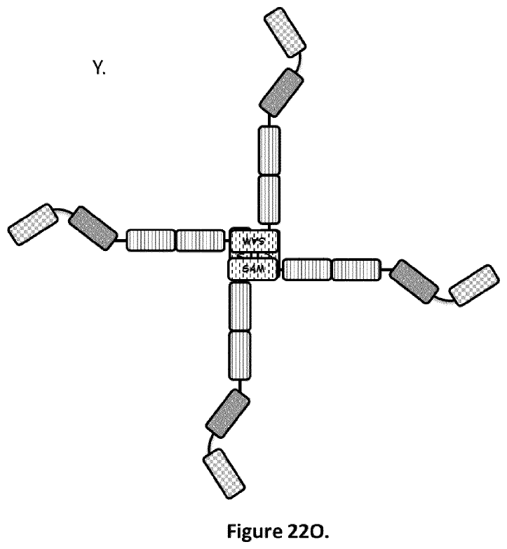

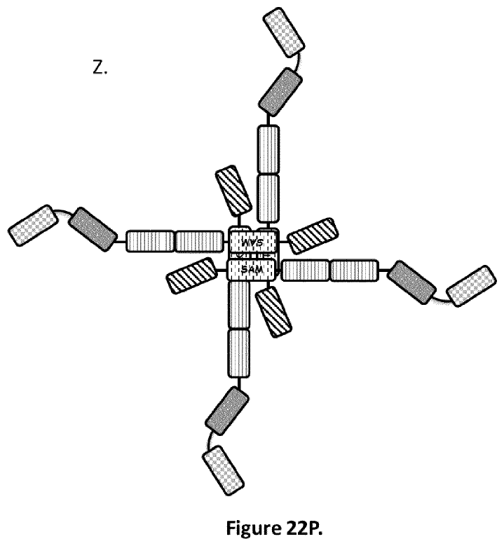

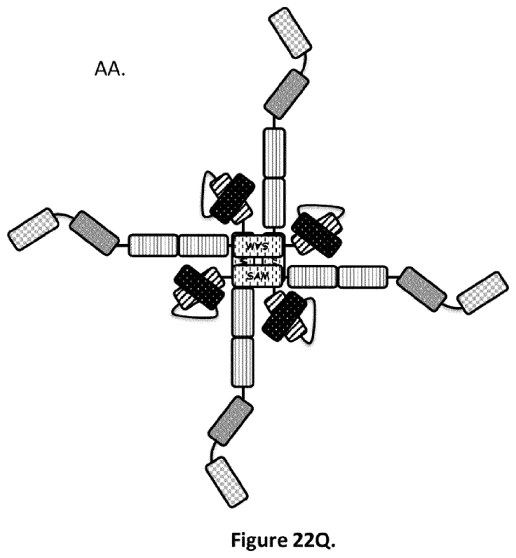

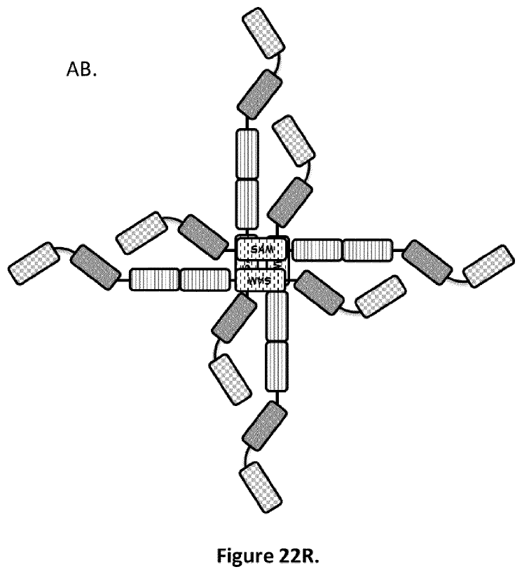

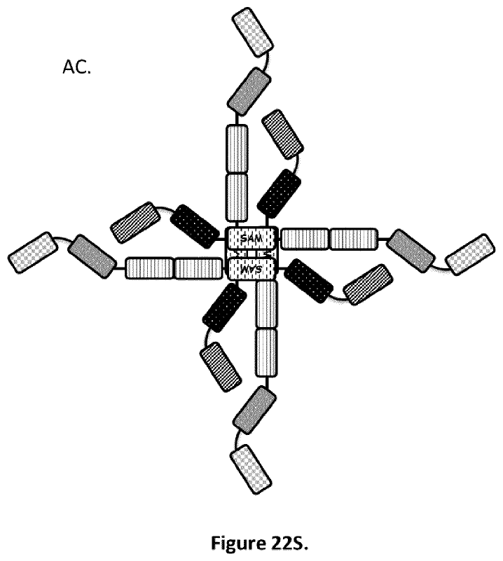

All DNA fragments were synthesized by Twist Bioscience (California) and cloned into the expression vector, pEF/myc/cyto (Invitrogen). Schematics and sequences of the synthesized DNA fragments are shown in FIG. 22 and Tables 8-10, respectively.

Plasmid DNA Preparation

Lyophilised plasmid DNA synthesized by Twist Bioscience, were resuspended with MQ water to a concentration of 50 ng/pl. Competent E. coli DH5α cells were transformed with 50 ng of DNA using a conventional heat shock method. Transformed cells were plated on LB agar plates containing 100 μg/mL ampicillin and grown overnight at 37° C. Individual colonies were picked and grown in LB broth overnight at 37° C., 220 rpm. Plasmid DNA were purified from the cells using Qiagen plasmid extraction kits, according to the manufacturer's instructions (Qiagen).

Expression of Quad Proteins in Expi293F Cells

Expi293F™ cells (Thermo Fisher Scientific) were cultured in Expi293™ Expression Medium (Thermo Fisher Scientific) according to the manufacturer's recommendations. The only exception was that 5% CO₂ was added directly to the flasks when the cells were split and non-vented caps were used.

Two methods involving different transfection reagents were utilised for protein expression. The methods for 30 ml cultures are described below and the protocol was adapted to either scaled up or down according to the experimental requirements.

For PEI transfections the cells were counted one day prior to transfection using a NC-3000m (ChemoMetec) and were diluted to 1.5×10⁶ cells/ml using Expi293™ Expression Medium. The cells were cultured in 5% CO₂ at 37° C., 125 rpm overnight. The following day the cells were counted, spun down for 5 minutes at 1000 rpm and resuspended at 2×10⁶ cells/ml in 30 ml of fresh media. 33 ug of plasmid DNA was added to 900 ul media and 90 ul of PEI Max (Polysciences Inc.) was added to 900 ul media. The DNA and transfection reagent samples were mixed and incubated at room temperature for 15 minutes. The DNA/transfection reagent mixture was added to the cells, which were cultured as before and incubated for a further 72 hrs.

For transfections with Expifectamine™ 293 Reagent (Thermo Fisher Scientific) the cells were also diluted to 1.5×10⁶ cells/ml in Expi293™ Expression Medium one day prior to transfection. On the day of transfection the cells were centrifuged and resuspended at 2.5×10⁶ cells/ml in 30 ml of fresh media. Two tubes containing 1.5 ml of Gibco™ Opti-MEM™ (Thermo Fisher Scientific) were prepared. 30 ug of plasmid DNA was added to one tube and 80 ul of Expifectamine was added to the other. The solutions were mixed and incubated at room temperature for 30 minutes. The DNA-transfection reagent complex was added to the cells, which were cultured in 5% CO₂ at 37° C., 125 rpm. Following 16-18 hrs incubation, transfection enhancers 1 and 2 were added to the cells according to the manufacturers protocol. The cells were incubated for a further 96 hours.

Purification of His-Tagged Proteins

The cells were harvested by centrifugation for 10 minutes at 4000 rpm. The ˜30 ml supernatant was filtered through a 0.22 μm filter and diluted to 50 ml with binding buffer (50 mM HEPES, pH 7.4, 250 mM NaCl, 20 mM imidazole) containing Complete™ EDTA-free protease inhibitors (Roche) to facilitate binding to the column. A 1 ml HisTrap™ HP column (GE Healthcare) was connected to an AKTA Start (GE Healthcare) and pre-equilibrated with binding buffer. The protein-containing media was loaded onto the column using a flow rate of 1 ml/min. The column was washed with >10 CV of binding buffer before the protein was eluted using a 20-300 mM imidazole gradient over 12 ml. 0.5 ml fractions were collected and analysed by SDS-PAGE. Protein containing fractions were pooled and concentrated using Amicon® Ultra centrifugal filter units (Millipore).

Following affinity chromatography the proteins were either snap frozen and stored at −80° C., dialysed into an alternative buffer for a specific application of gel filtrated to assess the molecular weight of the various Quad formats. For the latter analyses, protein samples were concentrated to 1.5-2 ml and gel filtrated on a Superdex 75 16/600 column (GE Healthcare) using 10 mM HEPES, pH 7.4, 250 mM NaCl.

SDS-Page

Purified proteins were analysed by separating out on SDS-PAGE under denaturing condition. Typically, 1-2 μg of purified protein were loaded per lane on SDS-PAGE gel. The gels were run in Tris-Glycine buffer containing 0.1% SDS. A constant voltage of 150 volts was used and the gels were run for ˜70 mins until the dye front has migrated fully.

SDS-PAGE (15% Bis-Tris) gels were prepared using the following resolving and stacking gels.

Resolving Gel:
5 ml 30% Bis-Acrylamide
2.6 ml 1.5 M Tris (pH 8.8)
50 pl 20% SDS
100 pl 10% APS
10 pl TEMED
2.2 ml MQ Water

Stacking Gel:
0.75 ml 30% Bis-Acrylamide
1.25 ml 1.5 M Tris (pH 8.8)
25 pl20% SDS
50 pl 10% APS
5 pl TEMED
2.9 ml MQ Water

General Direct Binding ELISA Assay

The potential of the purified Quad proteins to bind its target protein was confirmed by directing binding ELISA. Briefly, high binding 96 well plates (Corning) were used for coating recombinant target protein (1-5 ug/ml diluted in PBS or as indicated), which were typically stored at 4° C. overnight. Plates are then washed 3 times with 200 ul wash buffer (PBS+0.1% Tween) and blocked using 200 ul blocking buffer (PBS+1% BSA) for 1 hour at room temperature. Purified protein samples are typically serially diluted in dilution buffer (PBS+0.1% BSA) and 100 ul/well is added. Samples are incubated at room temperature for 1 hour after which the plate is washed again 3 times using 200 ul wash buffer. Anti-His HRP (Abcam) Detection antibody diluted according to the manufacturer recommendation is added and incubated at room temperature for 1 hour. The plate is washed for the final time using 3×200 ul wash buffer and 50 ul pre-warmed detection reagent (TMB-Sigma) is added per well and the plate incubated in the dark for 10-30 mins. The reaction is stopped by adding 25 ul/well of 1M sulfuric acid. The absorbance at 450 nm was read using a CLARIOstar microplate reader (BMG Labtech).

### Example 8: Expression of Monospecific Tetravalent dAb Quad

A multimer format (“Quad” format) where a single domain of an antibody variable fragment (dAb) (VH or VL either Vκ or Vλ) fused to p53 tetramerisation domain is exemplified in this example.

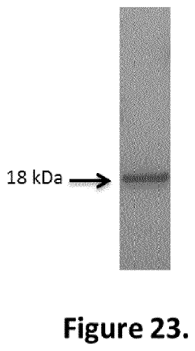

The dAb VH sequence for anti-IL17A (sequence adapted from WO 2010/142551 A2) was engineered into a tetravalent dAb Quad format (Quad 57) (Seq ID: 146). Expression vector for Quad 57 was synthesized by Twist Bioscience and expressed in HEK293 cells as described above. Secreted Quad 57 protein was collected from cell supernatant and purified using HisTrap HP column. To demonstrate soluble expression and purity of Quad 57, a small portion of the purified protein (1.5 ug) was separated out on SDS-PAGE gel (FIG. 23). A pure protein could be detected at the expected size (18 kDa) corresponding to Quad 57 confirming soluble expression of a tetravalent dAb Quad protein. A schematic representation of a tetravalent monospecific dAb Quad is shown in FIG. 22-A (ie, embodiment A of FIG. 22A).

### Example 9: Expression and Binding Analysis of Bispecific Tetravalent dAb Quad

A bispecific tetravalent dAb Quad is exemplified in this example where two different dAb binding domains are linked to p53 tetramerisation domain via the N- and C-terminus and as schematically represented in FIG. 22-C (ie, embodiment C of FIG. 22A).

In a specific example of a bispecific tetravalent dAb Quad, an anti-TNFa dAb VH binding domain from Ozoralizumab was linked to the N-terminus of p53 tetramerisation domain and an anti-IL17A dAb VH binding domain (sequence adapted from WO2010/142551 A2) was linked to the C-terminus of the p53 tetramerisation domain (Quad 54) (Seq ID: 143). Although in this specific example both the dAb binding domains were VH's, the dAb binding domains could be Vκ or Vλ or a combination of the different dAb formats.

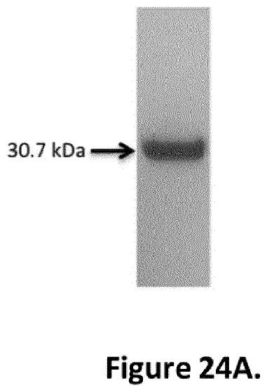

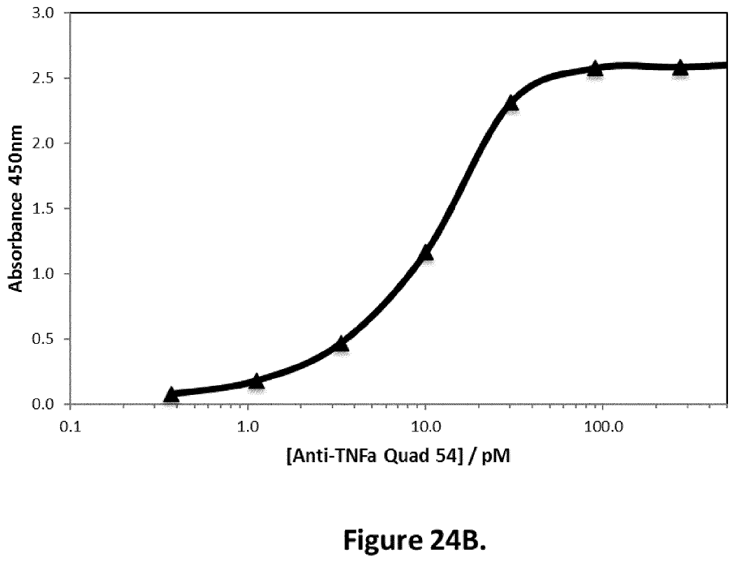

To demonstrate whether such a bispecific tetravalent dAb Quad format could be expressed as a soluble protein, expression construct containing dual anti-TNFa and anti-IL17A dAb binding domains were synthesized as a Quad format and expressed in HEK293 cells. Following protein purification from culture supernatant using HisTrap HP column, ˜1.5 ug of the purified protein was separated out on SDS-PAGE gel (FIG. 24A). From the SDS-PAGE gel, a pure product at the expected size (30.7 kDa) could be seen confirming expression of soluble bispecific tetravalent dAb Quad.

To further exemplify the functionality of such bispecific tetravalent anti-TNFa×anti-IL17A dAb Quad, direct binding ELISA assay was performed. High protein binding 96 well plates (coming) were coated with 1 ug/ml recombinant human TNFa protein (Abcam) and direct binding ELISA assay was performed with serially diluted Quad 54 protein using the method described above. A typical sigmoidal dose response curve was yielded from Quad 54 direct binding ELISA with recombinant human TNFa protein with a half-maximal binding at the low pM range (˜10 pM) (FIG. 24B). This confirms that not only can bispecific tetravalent anti-TNFa×anti-IL17A dAb Quads could be expressed as soluble proteins, they are also functional in that they can bind their target protein with high binding strength. The high binding strength is likely a measure of the increased avidity gained through tetravalent binding and this highlights an important feature of Quad multivalent molecules.

### Example 10: Expression, Binding and Functional Analysis of Monospecific Tetravalent scFv Quads

In this example, scFv binding domains for two different targets were selected and linked separately to the N-terminus of p53 tetramerisation domain to generate monospecific tetravalent scFv Quads as schematically represented in FIG. 22-D (ie, embodiment D of FIG. 22B).

In one example the scFv binding domain was an anti-TNFa where the VH and Vκ sequence was adapted from Humira into a scFv Quad format. To analyse whether the presence of a peptide linker effected the expression and binding of the Quad molecule to its target protein, in this example the anti-TNFa scFv binding domain was linked to the N-terminus of p53 tetramerisation domain either via a (G4S)3 peptide linker (Quad 63) (Seq ID: 147) or without a peptide linker (Quad 51) (Seq ID: 139).

In another example, the scFv binding domain was an anti-CD20 adapted from Wu et al., (Wu et al. 2001). The anti-CD20 scFv binding domain was engineered into a tetravalent Quad format by linking the binding domain directly to the N-terminus of p53 tetramerisation domain without a peptide linker (Quad 53 Tet) (Seq ID: 141).

All tetravalent scFv Quads were transfected into HEK293 cells and soluble protein from the culture supernatant were purified using HisTrap™ HP column as described above. A small amount of the purified protein (˜1.5 ug) was separated out on SDS-PAGE gels to confirm expression and purity.

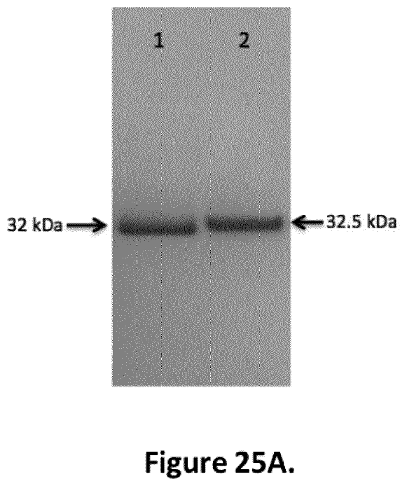

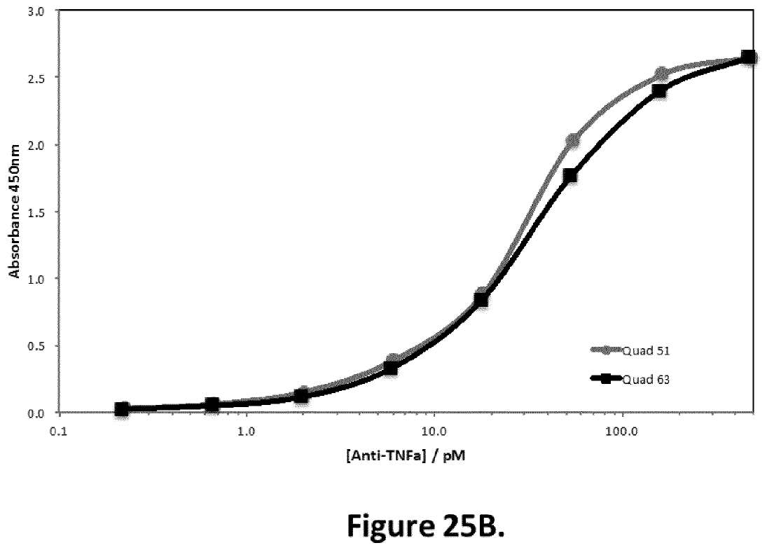

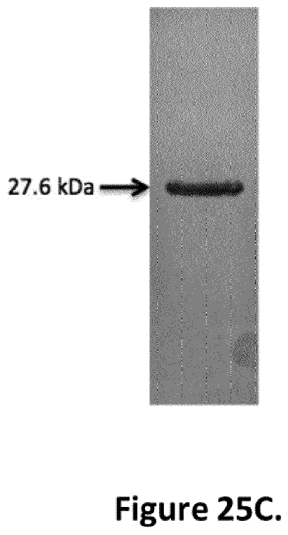

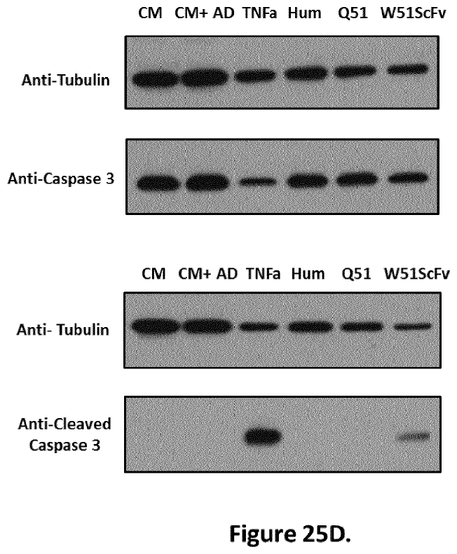

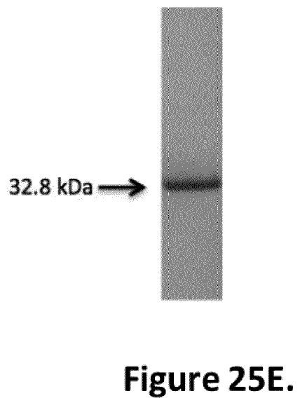

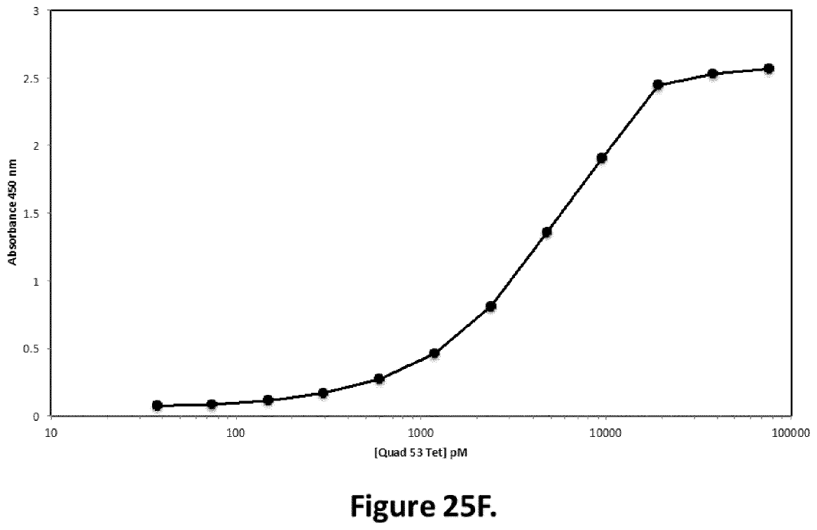

Anti-TNFa scFv Quads (Quads 51 & 63) were found to express well as soluble protein and the presence or absence of the peptide linker joining the scFv to the N-terminus of p53 tetramerisation domain did not appear to effect expression (FIG. 25A). Furthermore the purity of the secreted soluble Quad proteins appears to be highly pure (>99%). To characterize these Quads further, both Quad 51 and Quad 63 were analysed in a direct binding ELISA assay using recombinant human TNFa (Abcam). High binding 96 well plates (Costar) was coated with 1 ug/ml of human TNFa protein and ELISA binding assay was performed using serially diluted Quad 51 and Quad 63 proteins as described above. A dose-dependent increase in binding was observed for both Quad 51 and Quad 63 confirming these anti-TNFa scFv's were assembled correctly and their native binding functionality were retained in this Quad format. Furthermore, the binding profile for Quad 51 and Quad 63 was found to be highly similar with both Quads having a half-maximal binding concentration in the low pM range (˜30 pM) (FIG. 25B). Combinedly, these data confirm the presence or absence of the peptide linker does not effect protein expression or Quad binding strength to their target protein.

To further functionally characterize the activity of anti-TNFa Quad 51 molecule to neutralize TNFa, a cell-based assay was performed using WEHI-13VAR cells (ATCC), which is highly sensitive to TNFa. The bioassay using WEHI cells was set-up as described below. A monovalent anti-TNFa (W51ScFv) was included in the assay as a control (Seq ID: 150) and Humira was used as a positive control. W51ScFv was generated by modifying Quad 51 where the p53 tetramerisation domain was removed. Expression of W51ScFV was confirmed by SDS-PAGE (FIG. 25C).

Briefly, WEHI-13VAR cells were seeded at 1×10⁴ cells per well in a 96-well plate in RPMI-1640, 10% FBS and incubated overnight at 37° C., 5% CO₂. The media was aspirated from the cells and replaced with media containing 2 ug/ml actinomycin D, 0.1 ng/ml recombinant human TNFα (ab9649, Abcam) and 0-2400 pM Q51, Q35, W51ScFV and Humira. The samples were set up in quadruplicate with no TNFα and no antibody controls. The cells were incubated under standard culture conditions for a further 20-22 hours.

To assess cell viability, ATP generated by metabolically active cells was quantified using the CellTiterGlo Luminescent Cell Viability Assay (Promega) according to the manufacturers' instructions. Luminescent signals were measured using a CLARIOstar microplate reader (BMG Labtech). The luminescence signals obtained from the compound treated cells were normalised against the media on controls. The effective dose (ED) at which 50% of the WEHI cells retained viability was calculated and the ED50 for Quad 51, Humira and W51ScFV is summarised in Table 11.

As expected, Quad 51 having four anti-TNFa binding domains was found to be most effective at neutralising the cytotoxic effect of recombinant human TNFa protein on WEHI cells compared to Humira, which has two binding domains for TNFa. The W51ScFv control having only a single binding domain for TNFa had the highest ED50. The increasing valency of the anti-TNFa molecules for TNFa correlated inversely with a decrease in ED50 values. These data highlights the enhanced functional potency of Quad molecules with increasing valency and this aligns with the general concept of avidity verses potency as reported by numerous studies (Alam et al. 2018; Rudnick & Adams 2009; Adams et al. 2006; Brunker et al. 2016). Furthermore, the increase in avidity of Quad molecules can be used to drive selectively of tumour associated antigens that are highly expressed on tumour cells and thus limit the on-target off-tumour effect on healthy cells as reported in an example outlined by Slaga et al(Slaga et al. 2018).

To further demonstrate the ability of anti-TNFa Quad 51 to block TNFa induced activation of Caspase 3, Western blot analysis were performed alongside Humira and W51ScFv as controls. Briefly, WEHI-13VAR cells were seeded at 2.5×10⁶ cells per well in a 6-well plate in culture media (RPMI-1640, 10% FBS) and incubated overnight. The media was replaced with media containing 2 ug/ml actinomycin D, 1 ng/ml recombinant human TNFα and 500 pM of Quad 51, W51ScFV and Humira. Culture media only and culture media containing 2 ug/ml actinomycin D were included as controls. The cells were incubated with TNFα and +/−anti-TNFa molecules for 10 hours.

The cells were lysed using RIPA buffer (50 mM Tris, pH 8.0, 150 mM NaCl, 1% Triton X-100, 0.5% sodium deoxycholate, 0.1% SDS) containing 1 mM DTT and Complete™ EDTA-free protease inhibitor (Roche). Cell lysates were sonicated using a Bioruptor® Pico Sonication System (Diagenode) and the protein concentration of each sample was quantified using the Pierce BCA Protein Assay Kit (Thermo Fisher Scientific). 10 ug of protein was electrophoresed on a 12% Bis-Tris gel and bands were subsequently transferred to Amersham™ Hybond® P 0.45 μm PVDF membranes (GE Healthcare). The membranes were blocked with 10%-BSA-TBST or 5%-milk-TBST before being incubated overnight at 4° C. with appropriate antibodies (anti Caspase-3 (1/1000, CST, 9665) and anti-Cleaved Caspase-3 (1/100, CST, 9664). The membranes were washed with TBST and incubated with anti-rabbit IgG HRP-linked (1/2500, CST, 7074S) secondary antibody for 2 hours at room temperature. Following thorough washing, the membranes were developed using Pierce™ ECL Western Blotting Substrate (Thermo Fisher Scientific) and CL-XPosure™ films (Mermo Fisher Scientific).

From the Western blots, it can be seen that Quad 51 can effectively neutralise TNFa mediated activation of Caspase-3 with no detectable cleaved Caspase-3, similar to Humira (FIG. 25D). Whereas the monovalent W51ScFv anti-TNFa control molecule with high ED50 value, is not able to completely neutralize TNFa mediated activation of Caspase-3 and this is indicated by the presence of cleaved Caspase-3 in this sample. These data confirm that the mechanism of action of Quad 51 is through a signaling mechanism similar to Humira™.

In a second example, the anti-CD20 scFv Quad (Quad 53 Tet) was expressed in HEK293 cells and following purification, protein expression analysis and protein binding assays were performed. SDS-PAGE analysis confirmed soluble expression of Quad 53 Tet as a highly pure protein (>99%) (FIG. 25E). ELISA binding assay using recombinant human CD20 protein (Abcam) using serially diluted Quad 53 Tet protein confirmed a dose-dependent increase in binding. The ability of the anti-CD20 scFv Quad to retain binding to CD20 once again confirms correct assembly of this Quad format into a functional molecule (FIG. 25F).

### Example 11: Expression and Binding Analysis of Monospecific Octavalent scFv Quad

In this example, the valency of the scFv binding domain was increased from four (tetravalent) to eight (octavalent). This was achieved by linking an scFv to both N- and C-terminus of the p53 tetramerisation domain as schematically represented in FIG. 22-E.

As described in example 10, the anti-CD20 scFv binding domain was used in this example to construct a monospecific octavalent anti-CD20 scFv Quad (Quad 53 Oct) (Seq ID: 142). In this specific example, scFv's were linked to the N- and C-terminus of the p53 tetramerisation domain without peptide linkers, however, in other examples of this format, peptide linkers could be introduced at either or both ends to aid flexibility of the binding domain.

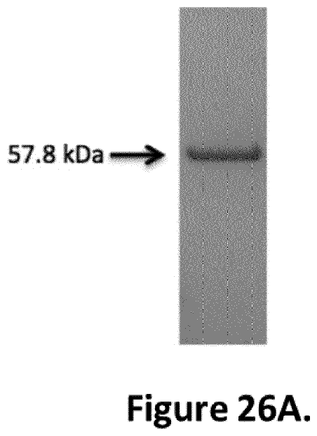

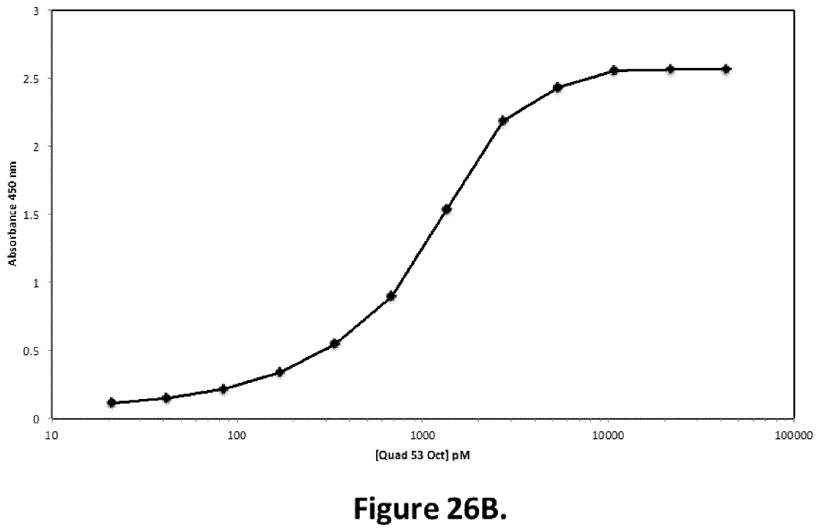

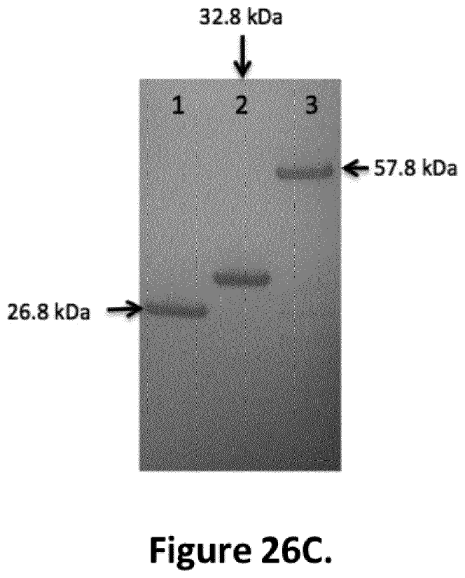

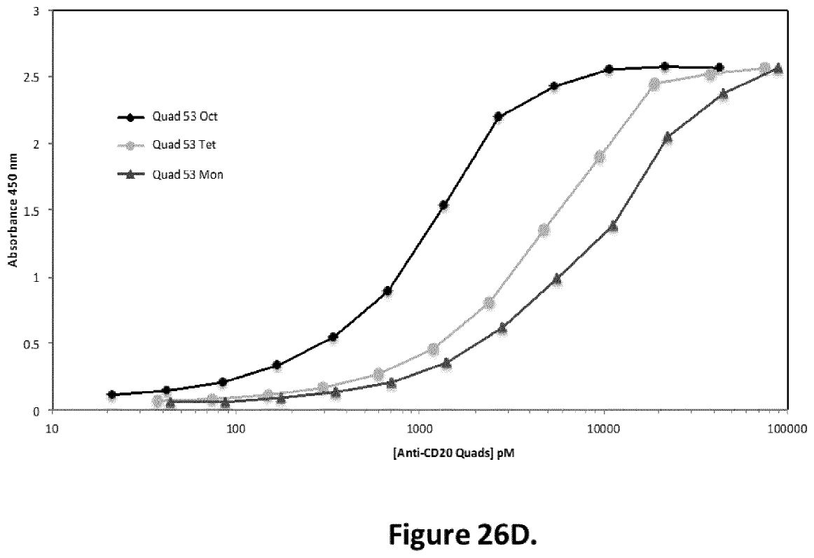

Following expression and purification of Quad 53 Oct protein from culture supernatant, protein expression analysis and ELISA binding assays were performed. Protein separated out on SDS-PAGE gel confirmed soluble expression of Quad 53 Oct with high purity (>99%) (FIG. 26A). ELISA binding assay was also performed using recombinant CD20 and serially diluted Quad 53 Oct protein (FIG. 26B). A dose-depended increase in binding could be seen confirming correct assembly of anti-CD20 scFv in an octavalent format.

To analyse the effect of increasing valency on target protein binding, scFv anti-CD20 without the tetramerisation domain was constructed to act as a monovalent control (Quad 53 Mon) (Seq ID: 140). Following protein expression and purification, all three Quad 53 anti-CD20 scFv molecules (monovalent, tetravalent and octavalent) were separated out side-by-side on a SDS-PAGE gel (FIG. 26C) and ELISA binding assay was performed (FIG. 26D). With all three Quad 53 molecules, a dose-dependent increase in binding could be seen and the overall increase in binding strength was reflected by the increase in the valency. The octavalent anti-CD20 scFv version was found to have the highest overall binding strength, followed by the tetravalent and then the monovalent version. It is noteworthy, due to the nature of direct binding ELISA where the target protein is densely coated onto 96 well plates resulting in rapid Quad 53 binding saturation to CD20, the true avidity (total binding strength) can not be accurately determined using this method. Despite these limitations of direct binding ELISA, a representative increase in binding could still be observed with increasing valency as seen in FIG. 26D.

### Example 12: Expression and Binding Analysis of Bispecific Tetravalent scFv Quad

In this example, two different scFv binding domains with specificity for two different target proteins were linked to the p53 tetramerisation domain via the N- and C-terminus to give a bispecific tetravalent scFv Quad as exemplified schematically in FIG. 22-F.

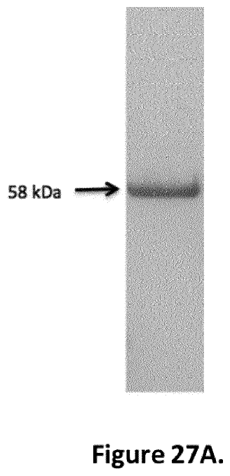

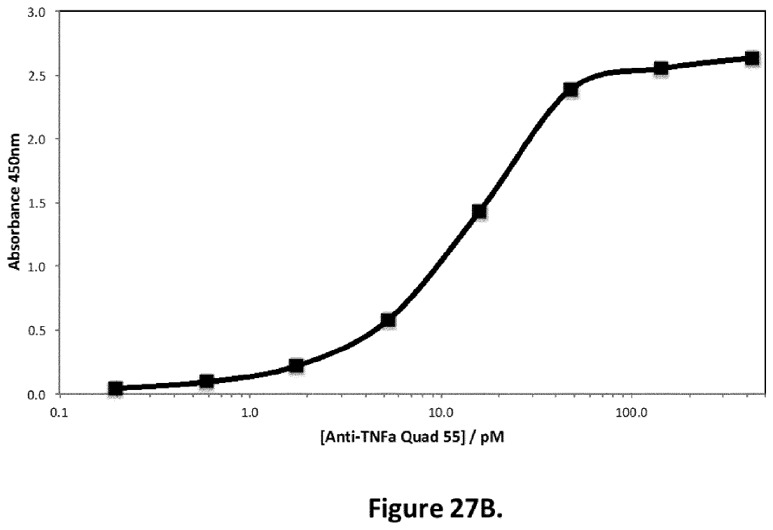

The specific scFv binding domains used in this example has specificity for anti-TNFa and anti-IL17A. The anti-TNFa scFv sequence was adapted from Humira and the anti-IL17A scFv sequence was adapted from Ixekizumab (Eli Lilly) into a bispecific Quad format (Quad 55) (Seq ID: 144). To confirm soluble expression, Quad 55 was expressed in HEK293 cells and the secreted protein was purified from culture supernatant followed by protein analysis. (FIG. 27A). Protein analysed by SDS-PAGE confirmed soluble expression of this bispecific scFv Quad format with high purity (>99%). To further analyse whether this format was functional, ELISA binding assay was performed for the anti-TNFa scFv arm (FIG. 6B). A dose-dependent increase in binding of Quad 55 to recombinant human TNFa could be seen confirming Quad 55 was assembled correctly. Furthermore, Q55 appears to have a similar half-maximal binding in the low pM range to that of Quad 51 detailed in Example 10.

### Example 13: Expression and Binding Analysis of Bispecific Tetravalent scFv×dAb Quad

In this example, two different binding domain formats with specificity for two different target proteins were linked to the N- and C-terminus of p53 tetramerisation domain respectively, as exemplified schematically in FIG. 22-G.

The first binding domain was an anti-TNFa scFv as detailed in Example 12, which was linked to the p53 tetramerisation domain via the N-terminus. The second binding domain was an anti-IL17A dAb sequenced, which was linked to the p53 tetramerisation domain via the C-terminus as detailed in Example 9 (Quad 56) (Seq ID: 145).

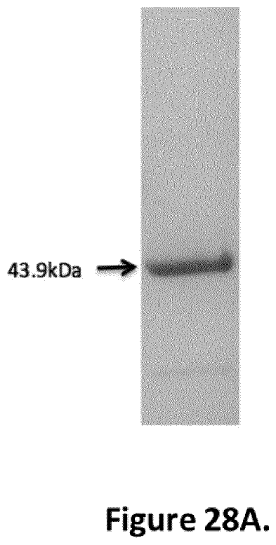

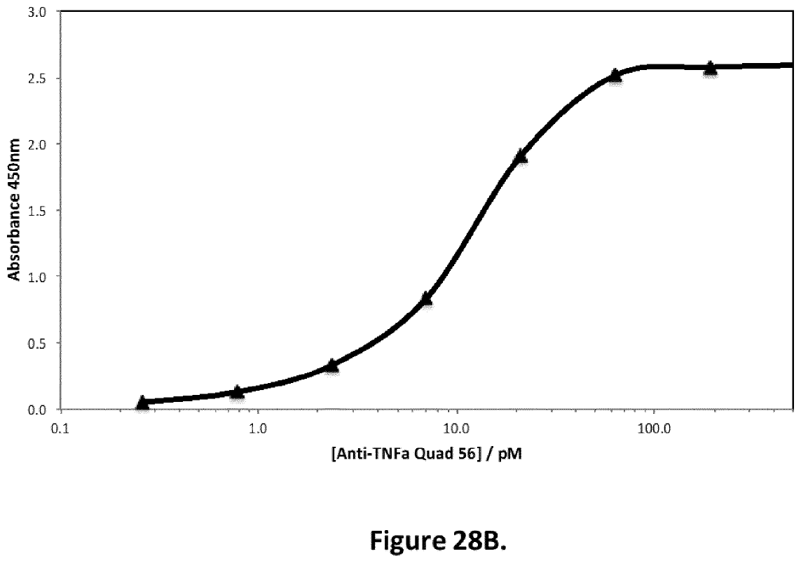

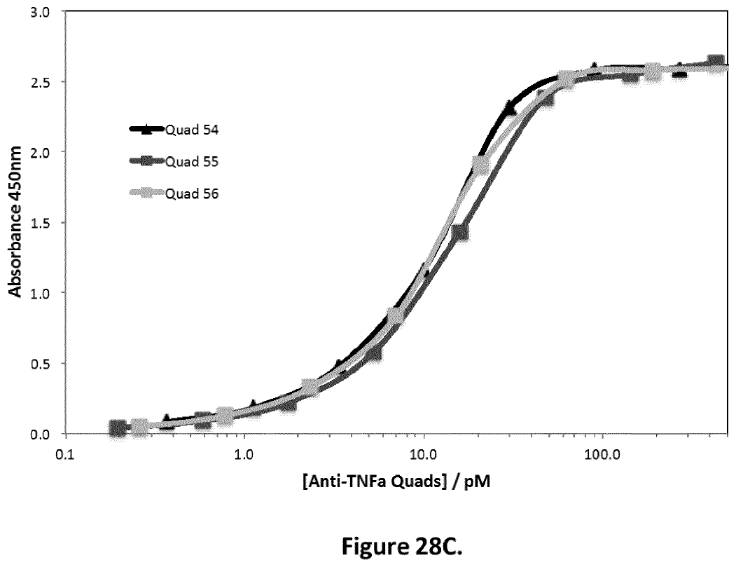

Soluble expression of this bispecific tetravalent scFv×dAb Quad format was confirmed by analysing the purified protein on SDS-PAGE gel (FIG. 28A). In additional ELISA binding assay was performed for the anti-TNFa binding arm (FIG. 28B), where a dose-depended increase in binding was observed. This confirmed correct expression and assembly of this bispecific scFv×dAb Quad format.

In the examples above (Examples 10, 12 & 13) different bispecific Quad formats (Quads 54-56) with specificity for anti-TNFa×anti-IL17A were produced and analysed for expression and functionality by ELISA binding assay for the anti-TNFa binding arm. From the data presented thus far, it is clear that the p53 tetramerisation domain is highly versatile and amenable to fusion with different binding domains at either or both N- and C-terminus. To compare the functional binding strengths of Quads 54-56, the ELISA binding assay data for the anti-TNFa binding arm of the different bispecific Quads were plotted side-by-side (FIG. 28C). The dose-response curves for all three bispecific Quad formats appear to be highly similar indicating that the binding domain format (scFv or dAb) does not affect binding. This further highlights the versatility and utility of the p53 tetramerisation domain to generate highly pure multivalent soluble proteins with high binding strength.

### Example 14: Expression and Binding Analysis of Tetravalent Monospecific Ig scFv Quad v1

In this example, an scFv binding domain was linked to the lower hinge/CH2 domain of IgG1 Fc without the core and upper hinge region to generate a scFv monomeric Ig Fc (scFv-mFc), ie wherein the Fc does not pair with another Fc when a multimer is formed using the polypeptide monomer. Typically a CXXC motif comprising cysteine residues present in the core hinge region is responsible for forming inter-chain disulfide bonds. Thus, by excluding the core hinge region, the Fc region is restrained from forming a tightly packed homodimer structure typically found in native IgG antibodies. The lower hinge/CH2 domain was kept intact to allow proper interaction with Fcγ-receptors required for effector function.

The scFv-mFc was engineered into a Quad format by linking it to the p53 tetramerisation domain via the N-terminus to generate a tetravalent monospecific Ig scFv Quad termed version 1 as schematically represented in FIG. 22-I (ie, embodiment I shown in FIG. 22D).

In this example the upper hinge was also removed, but in other examples the upper hinge region can be optionally retained completely or only partially kept intact to generate dAb monomeric Ig Quads (exemplified schematically in FIG. 22-H, L, W, X, Y, Z, AA, AB & AC), scFv monomeric Ig Quads (exemplified schematically in FIG. 22-I, M, X, & AA) and Fab monomeric Ig Quads (exemplified schematically in FIGS. 22-J, K, N & O) with monospecific, bispecific, trispecific or tetraspecific specificity. Although in this specific example, human IgG1 was used as an example, the Fc region could be any of the other IgG isotypes including IgG2, IgG3, gG4 or derivative thereof. The amino acid sequences encoding human IgG hinge regions are shown in Table 12.

The scFv binding domain used in this specific example was that of an anti-CD20 described in Example 10. The anti-CD20 scFv was linked to the lower hinge/CH2 domain of the Fc via a peptide (G4S)3 linker. The p53 tetramerisation domain was linked directly to the C-terminus of the CH3 domain without any peptide linkers (Quad 64) (Seq ID: 148). An optional linker could be included at this junction between the CH3 domain and the multimerisation domain to provide flexibility.

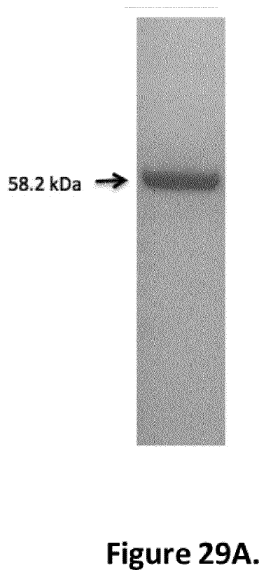

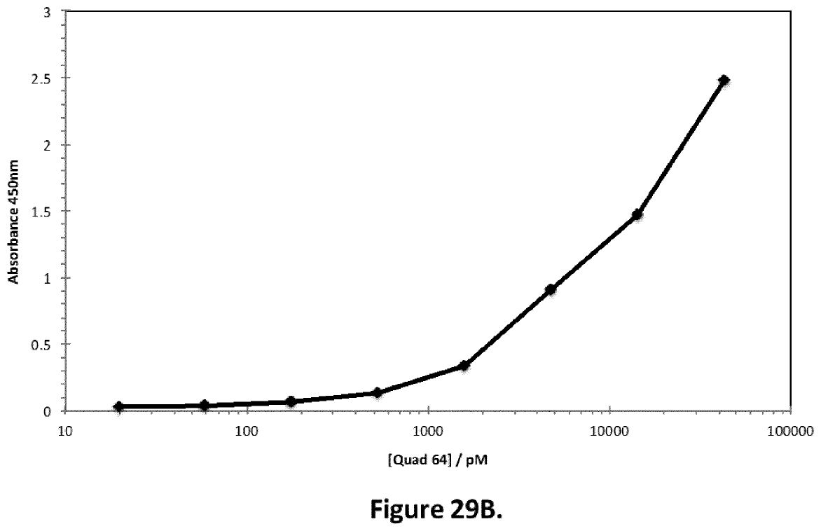

Following Quad 64 expression and purification, protein was quantified and analysed by SDS-PAGE (FIG. 29A). Protein quantification using Nanospec confirmed high protein yield after HisTrap HP column purification with protein yield equivalent to 130 mg/L. In addition, a single protein band at the expected size (58.2 kDa) as seen on the SDS-PAGE gel confirmed expression of a highly pure (>99%) tetravalent monomeric Ig scFv Quad protein. To analyse whether the expressed Ig Quad protein was assembled correctly, ELISA binding assay was performed (FIG. 29B). Quad 64 was found to retain binding to recombinant human CD20 in a dose-dependent manner confirming that it was assembled correctly and functionally active.

### Example 15: Expression and Binding Analysis of Tetravalent Monospecific IE scFv Quad v2

In this example, a different version of the tetravalent monomeric Ig scFv Quad described in Example 14 was constructed where the anti-CD20 scFv binding domain was linked directly to the N-terminus of the p53 tetramerisation domain. The mFc containing the lower hinge, CH2 and CH3 domains was directly linked to the C-terminus of the p53 tetramerisation domain. This version of tetravalent monomeric Ig scFv Quad is termed version 2 and is schematically represented in FIG. 22-M (ie, embodiment M shown in FIG. 22F).

Although in the specific example described below, the upper hinge was not included, in other examples the upper hinge region can be optionally retained completely or only partially kept intact.

In other examples, the version 2 configuration could contain dAb binding domains as exemplified schematically in FIG. 1-L, X, Z & AA, producing Quad molecules with monospecific, bispecific or trispecific specificity. In another example, the binding domain could be a Fab as exemplified schematically in FIGS. 22-N & O. In yet another example, version 2 could be made without mFc to give tetravalent Fabs either as monospecific (exemplified schematically in FIGS. 22-Q & R) or bispecific (exemplified schematically in FIG. 22-S).

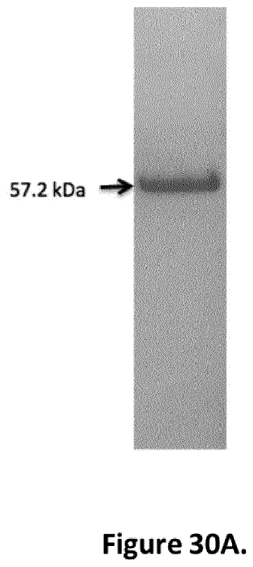

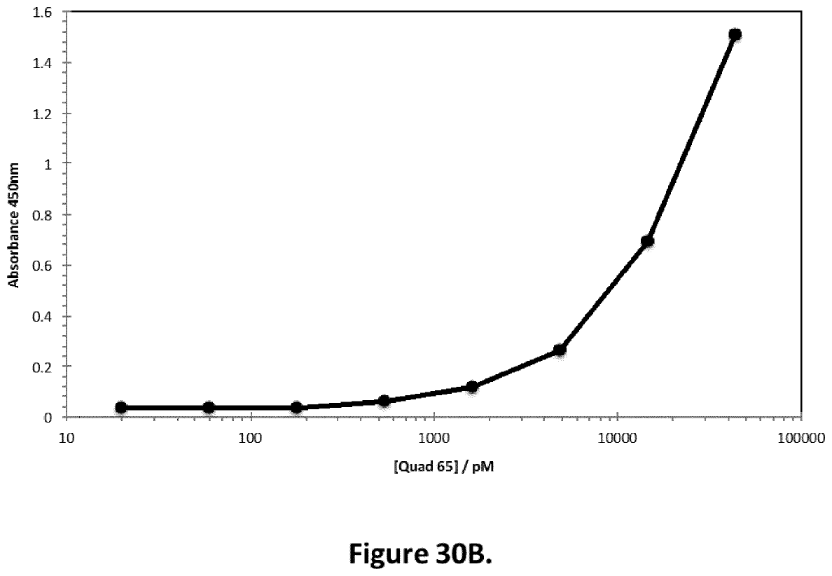

The expression construct for the tetravalent monomeric Ig scFv Quad version 2 specific for CD20 (Quad 65) (Seq ID: 149) was expressed in HEK293 cells and the soluble secreted protein was analysed by SDS-PAGE. Protein quantification using Nanospec confirmed high protein yield after HisTrap HP column purification with protein yield equivalent to 160 mg/L. In addition, a single protein band at the expected size (57.2 kDa) as seen on the SDS-PAGE gel confirmed expression with high purity (>99%) (FIG. 30A). The integrity of the expressed protein was analysed by ELISA binding assay. Recombinant human CD20 protein was used in a direct binding ELISA using serially diluted Quad 65 protein. Quad 65 was found to bind CD20 in a dose-dependent manner (FIG. 30B) confirming it assembled correctly and functionally active.

The data outlined above and in Example 14, represents the first examples of soluble and functional expression of tetravalent monomeric Ig molecules. Such multivalent monomeric Fc formats would have several advantages over scaffold and antibody fragment based molecules lacking Fc. Firstly, the presence of an Fc region in a monomeric Quad format would allow neonatal Fc receptor (FcRn) binding providing an extended half-life of these molecules in-vivo. Secondly, the presence of an Fc region would have the ability to bind multiple Fc receptors to induce effector functions such as antibody-dependent cell-mediated cytotoxicity (ADCC) and complement-dependent cytotoxicity (CDC) as reported for monovalent monomeric Ig Fc molecules (Ying et al. 2017; Ying et al. 2012). Thirdly, the proof-of-concept data outlined in this example suggest that any given monoclonal IgG antibody could be rapidly formatted into a multivalent monomeric Ig Quad format as schematically represented in FIGS. 22-J, K, N & O). With these formats retaining the native binding domain architecture of an IgG antibody, multivalent monomeric Ig Quad formats would have substantially increased binding strength for its target protein due to the increased avidity gained through multivalency. Such monomeric Ig Quad format will provide novel class of therapeutic molecules with enhanced target protein neutralization potential (Boruah et al. 2013; Shen et al. 2019) as well as having enhanced efficiency to cross-link cell surface receptor to mediate apoptosis (Li et al. 2018) and induce signaling through receptor super-clustering with enhanced agonistic potentials (Mayes et al. 2018).

### Example 16 Expression of Octavalent Bispecific Quads

In a specific example of an octavalent bispecific Quad, dAb binding domains for PD-L1 and 4-1BB were used for the dual targeting of an immune checkpoint inhibitor and an immune co-stimulatory molecule for the treatment of cancers. In other examples, dAb binding domains could have specificity for any immune checkpoint inhibitor and any co-stimulatory molecule or a mixture thereof to provide either a dual checkpoint inhibitor bispecific multimer (e.g. PD-L1×CTLA-4) or a dual checkpoint inhibitor and immune co-stimulatory bispecific multimer (e.g. PD-L1×4-1BB) or a dual immune co-stimulatory bispecific multimer (e.g. 4-1BB×OX40).

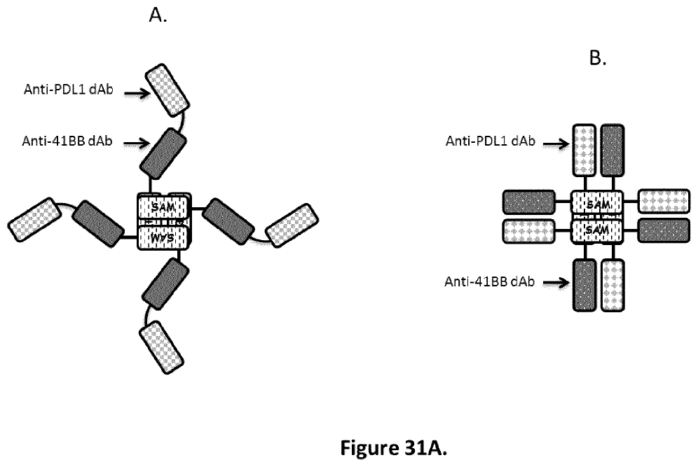

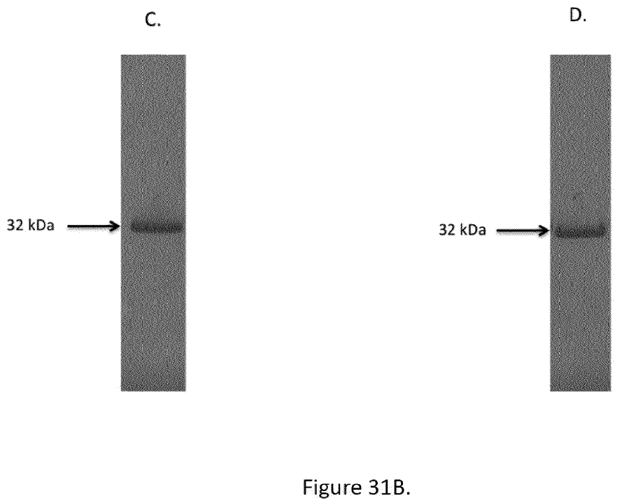

To exemplify octavalent bispecific Quads, two different versions were constructed to demonstrate soluble Quad protein expression. In one format, the PD-L1 and 4-1BB dAb binding domains were linked to a p53 multimerisation domain as a tandem dAb at either the N- or C-terminus as schematically represented in FIG. 31-A.

In a second example of an octavalent bispecific multimer, the dAb binding domains for PD-L1 and 4-1BB were linked to the p53 tetramerisation domain at opposite ends respectively as schematically represented in FIG. 31-B.

The dAb binding domain sequence for PD-L1 and 4-1BB were adapted from WO2017/123650A2. In the first version of an octavalent bispecific Quad, a tandem dAb containing anti-PD-L1 and anti-4-1BB in a N-C terminus orientation was linked to the N-terminus of p53 tetramerisation domain without any peptide linkers. This version is referred to as Quad 68 (SEQ ID NO: 190). However, in other examples, the tandem dAb can be linked to the tetramerisation domain via an optional peptide linker and as further described in Table 8-P.

In a second version of octavalent bispecific Quad multimer, the anti-PD-L1 dAb was linked to the N-terminus of p53 tetramerisation domain and the anti-4-1BB dAb was linked to the C-terminus. This version is referred to as Quad 69 (SEQ ID NO: 191). In this specific example the dAbs were linked to the tetramerisation domain without any peptide linkers. However, in other examples, dAbs can be linked to the tetramerisation domain via an optional linkers and as further described in Table 8-C.

The expression construct for Quads 68 and 69 were expressed in HEK293 and the secreted soluble proteins were purified from cultured supernatant. The purified Quad proteins were analysed by SDS-PAGE (FIGS. 31-C & D). Protein quantification of Quad 68 and Quad 69 using Nanospec confirmed high protein yield after HisTrap HP column purification with protein yield equivalent to 297 mg/L and 360 mg/L respectively. The presence of a single protein band for both Quad 68 and Quad 69 at the expected size (32 kDa) as seen on the SDS-PAGE gel confirmed expression with high purity (>99%) (FIGS. 31-C and D).

The configuration of the dAbs either in tandem (as in Quad 68) or in opposite orientation (as in Quad 69) are anticipated to engage with cancer cells via PD-L1 with higher potency and selectively than to T cells expressing 4-1BB. Therefore, T cells will preferentially be recruited to cancer cells only once the cancer cells are engaged with the Quad molecule allowing selective T cell activation with improved safety profile.

### Example 17: Expression of Tetravalent Monomeric Ig dAb Quad and Octavalent Fab-Like dAb Monomeric Ig Quad

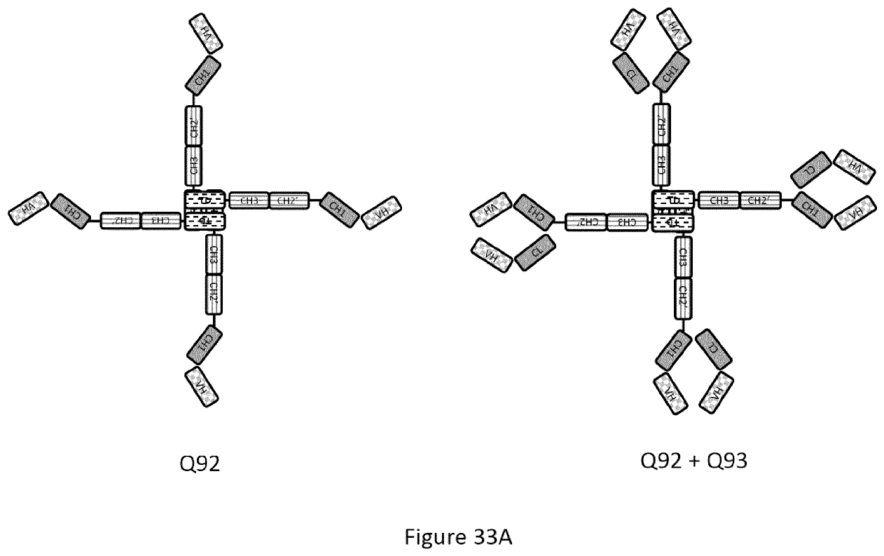

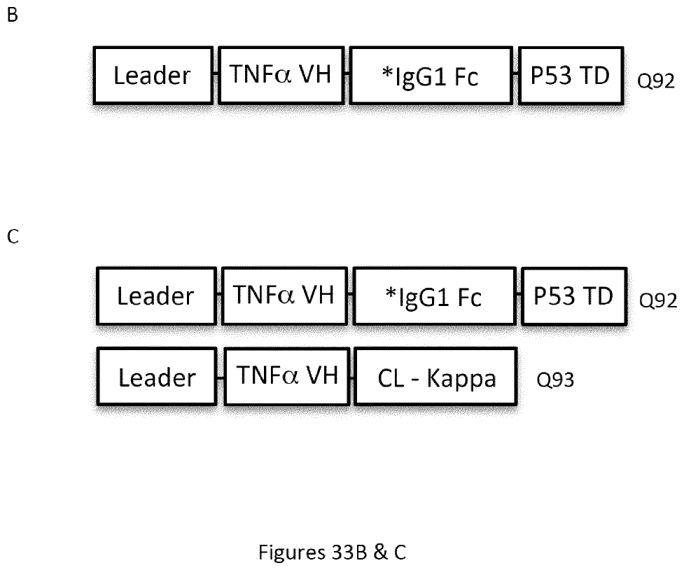

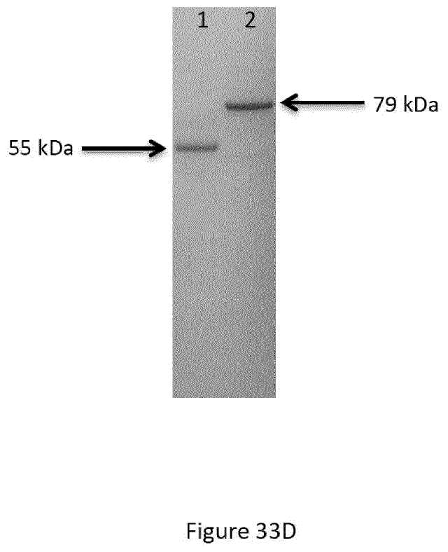

A dAb VH with specificity for TNFα as detailed in Example 9 was also used in this example to generate two new versions of monomeric Ig dAb Quads. In the first version, anti-TNFα dAb VH was linked directly to IgG CH1. The CH1 region was linked to the IgG1 Fc where the hinge region was modified so that the core hinge was removed (SEQ ID NO: 168 hinge sequence was used). The Fc region was linked to the N-terminus of the p53 TD domain yielding Quad 92. The presence of CH1 region in Q92 allowed for the generation of a second version of monomeric Ig Quad where Q92 when co-expressed with anti-TNFα dAb linked to Kappa light chain constant domain yielded an octavalent anti-TNFα Fab-like dAb monomeric Ig Quad (Q93) as schematically represented in FIG. 33A. The molecular design of Q92 and Q93 is schematically represented in FIGS. 33B&C.

Q92 alone or Q92 plus Q93 were expressed in HEK293 cells and the Quad proteins were purified directly from the culture supernatant. The purified proteins were analyzed by SDS-PAGE where pure products at the expected molecular weight (Q92 (tetravalent)—55 kDa and Q92+Q93 (octavalent)—79 kDa) could be seen confirming soluble expression of these Quad formats (FIG. 33D). Although in this specific example, the dAb tetravalent version retained the CH1 region, in another example the CH1 domain could be removed to generate a slightly modified version of tetravalent dAb Quad as schematically represented in FIG. 22F (embedded in Figure as L).

### Example 18: Expression of Monospecific and Bispecific Octavalent Tandem dAb Monomeric IE Quads

In this example, two different versions of monomeric Ig Quads were generated similar to that exemplified in Example 14. However, instead of using scFv as binding domains, in this specific example antibody single domains (VH) were linked in tandem with specificity for either PDL1 alone or PDL1 plus 4-1BB. In the first of these examples, a bispecific octavalent anti-PDL1/4-1BB monomeric Ig dAb Quad (Q113) was generated by linking in tandem an anti-PDL1 dAb with an anti-4-1BB dAb separated by a flexible linker. This tandem bispecific binding module was linked to the lower hinge/CH2 region of IgG1 Fc without the core hinge region to generate a tandem dAb monomeric Ig Fc (ie, the resulting polypeptide comprised (in N- to C-terminal direction) an anti-PDL1 dAb, an anti-4-1BB dAb, lower hinge/CH2 region of IgG1 Fc without the core hinge region and IgG1 CH3). The Fc region was linked to the N-terminus of p53-TD domain to generate a bispecific tandem dAb monomeric Ig Fc Quad as schematically represented in FIG. 220. In this example, the binding valency of both the anti-PDL1 and anti-4-1BB dAb domains were tetravalent for their target protein. This is an example of a multivalent 4+4 (octavalent) bispecific antibody containing Ig Fc region. The second example was exactly the same as Q113, except the tandem dAb consisted only of anti-PDL1 dAb domains generating an octavalent anti-PDL1 monomeric Ig Quad (Q114).

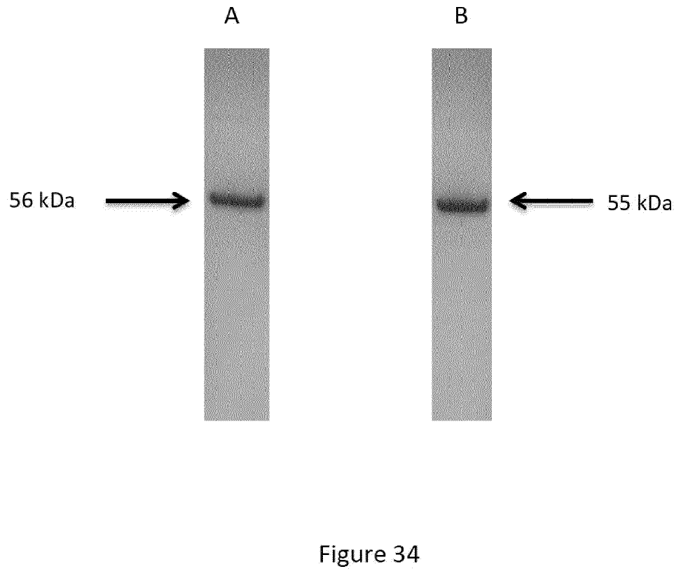

Following Quad 113 and Q114 expression in HEK293 cells and purification of proteins from culture supernatant, the recovered proteins were analysed by SDS-PAGE (FIGS. 34A & B). For both Q113 and Q114, a single pure protein band at the expected molecular weight was observed confirming expression of these monomeric Ig Quads as highly pure (>99%) soluble Quads proteins.

### Example 19: Expression of Fab Monomeric Ig Quad

In this specific example, monomeric Ig Quads were produced from IgG monoclonal antibodies where the native pairing of the variable heavy chain (VH) and variable light chain (VL) was kept intact. This was achieved by taking the Fab fragment of IgG monoclonal antibody and converting it into a monomeric Ig Quad as schematically represented in FIG. 22E (embedded in Figure as J). To exemplify such Quad format, Fab fragments from two clinically approved monoclonal antibodies were used (adalimumab (Humira™) and avelumab (Bavencio™)). The VH-CH1 domain of either Humira or Avelumab was linked to the lower hinge/CH2 region of IgG1 Fc without the core hinge region. The Fc region was linked to the N-terminus of p53-TD domain to generate the monomeric Ig Quad chain. To generate Humira and avelumab Fab monomeric Ig Quad, the native light chain VL-CL was co-expressed with the respective monomeric Ig Quad chain in HEK293 cells.

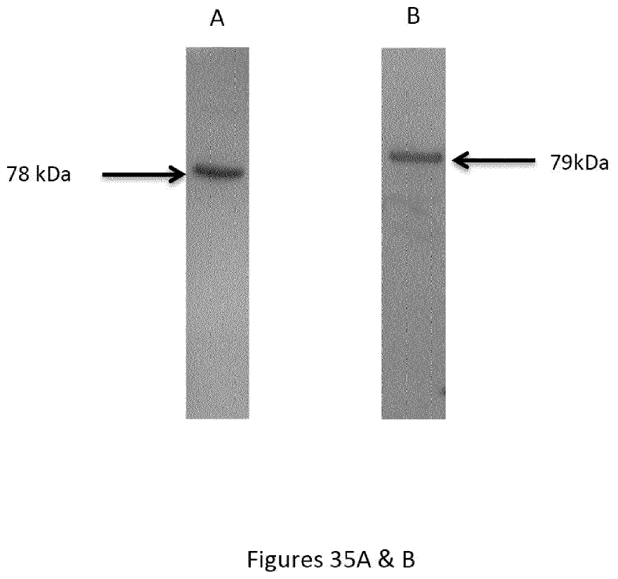

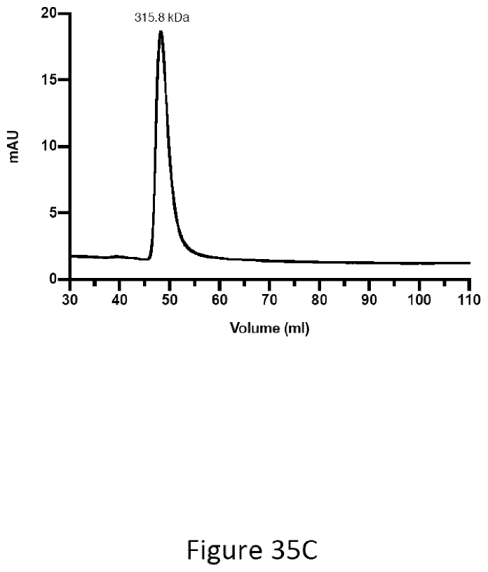

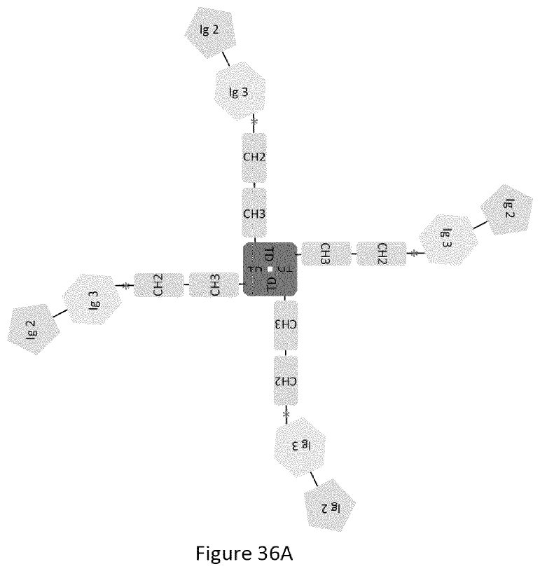

Fab monomeric Ig Quad proteins were purified from the culture supernatant and analysed initially by SDS-PAGE (FIGS. 35A & B). Detection of a single protein band at the expected molecular weight on a non-reduced denaturing SDS-PAGE gel corresponding to either Humira or avelumab Fab monomeric Ig Quad, confirmed both soluble expression and high purity of these monomeric Ig formats. The absence of any detectable free HC or free LC in these Fab monomeric Ig Quad protein preps further confirmed the high stability of these formats. To confirm the native molecular mass and oligomeric state of these Fab monomeric Ig Quad proteins, Humira Fab monomeric Ig Quad protein was analysed by size-exclusion chromatography (FIG. 35C). The size exclusion chromatography profile for Humira Fab monomeric Ig Quad protein had a clear dominant peak eluted at the expected volume consistent with the tetrameric molecular weight of 315.8 kDa. These data further confirm the high purity of these Quad proteins, which are stable and present in a homogeneous tetrameric form without the presence of any aggregates. Further examples of Fab monomeric Ig Quad could be generated from any given monoclonal antibody where the intact Fab is used to generate Quads without altering the native VH and VL pairing or it's native antigen binding potential.

### Example 20: Expression of Extracellular Protein Domain as Monomeric Ig Quads (Q96)

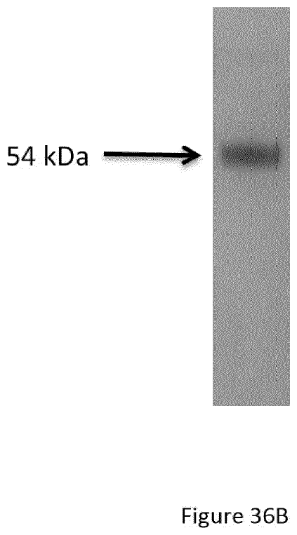

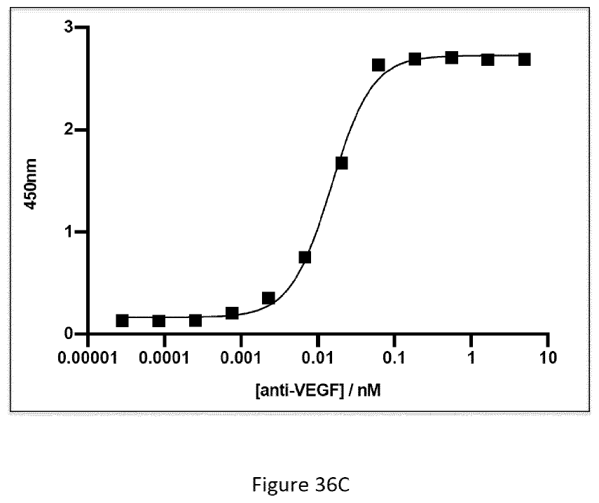

In the above examples, antibody fragments were used to generate Quads. In this specific example the versatility of p53 TD domain was demonstrated further whereby extracellular domains of cell surface receptors were multimerised into a Quad format without affecting its ability to bind its natural ligand. To exemplify this, the soluble extracellular domains used in the VEGF trap aflibercept (Eylea™) was used as an example. The Ig domain 2 from VEGFR1 and Ig domain 3 from VEGFR2 similar to Eylea was linked to monomeric Ig Quad similarly to that exemplified in Examples 17-19 and as schematically represented in FIG. 36A (Q96).

Q96 was expressed in HEK293 cells and soluble protein was purified directly from the culture supernatant. SDS-PAGE analysis confirmed expression of Q96 as a highly pure Quad with a single protein band at the expected molecular weight (FIG. 36B). The integrity of Q96 was analysed further by ELISA binding assay as described above to confirm binding to VEGF-A ligand. ELISA plates were coated with VEGF-A at 0.5 ug/ml and serially diluted (1 in 3 folds diluted starting from 5 nM) Q96 protein was added to the coated VEGF-A plate. Anti-His HRP detection antibody was used for detection of Q96 binding to VEGF-A. Q96 bound VEGF-A in a dose dependent manner confirming this format containing extracellular domains of VEGFR1 and VEGFR2 assembled correctly and it retained it's native ligand binding potential (FIG. 36C).

### Example 21: Expression, Binding and Functional Potency of Non-Ig Tetravalent and Non-Ig Octavalent Anti-TNFα dAb Quads

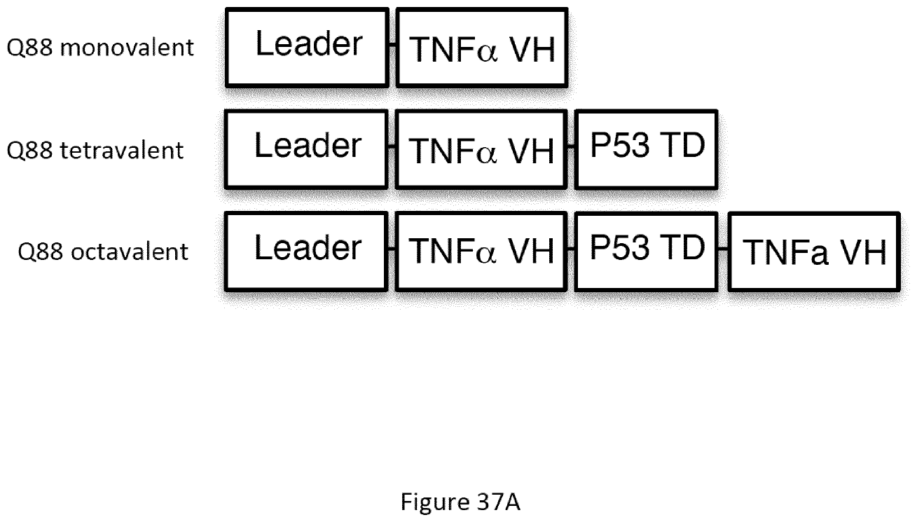

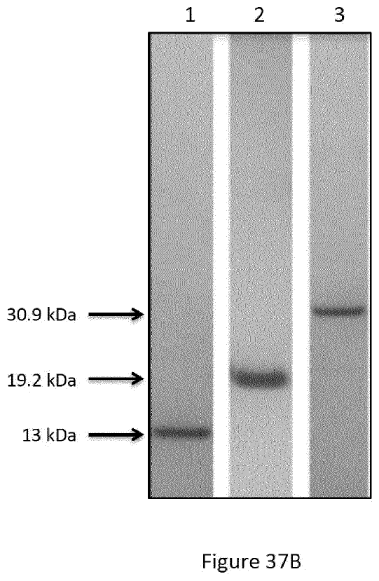

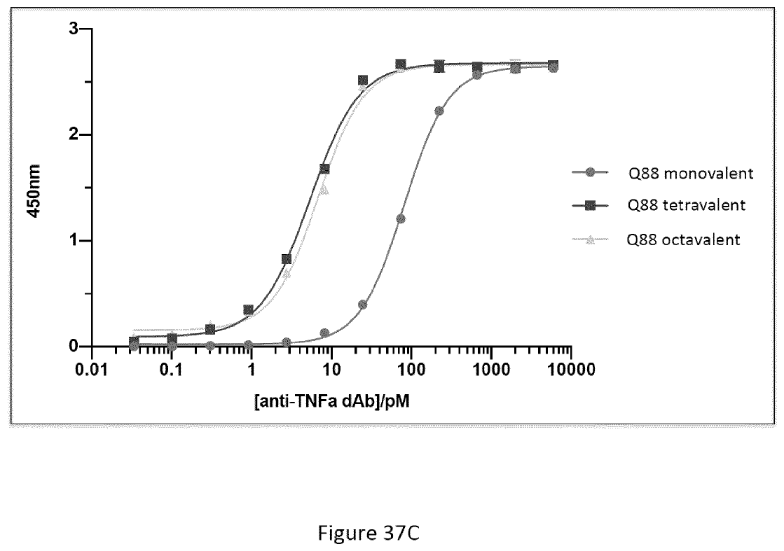

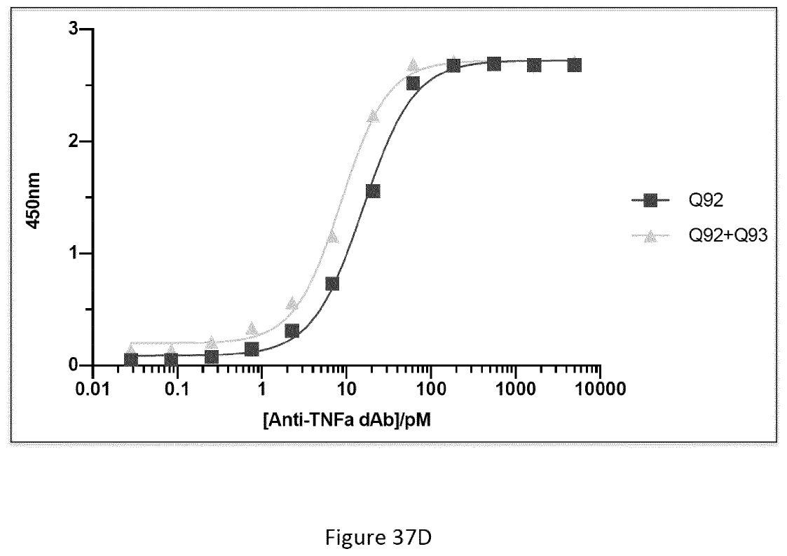

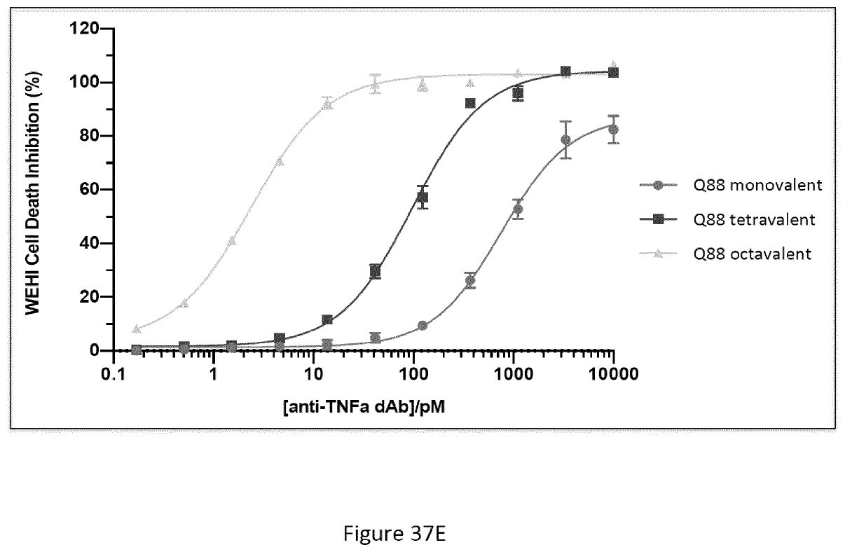

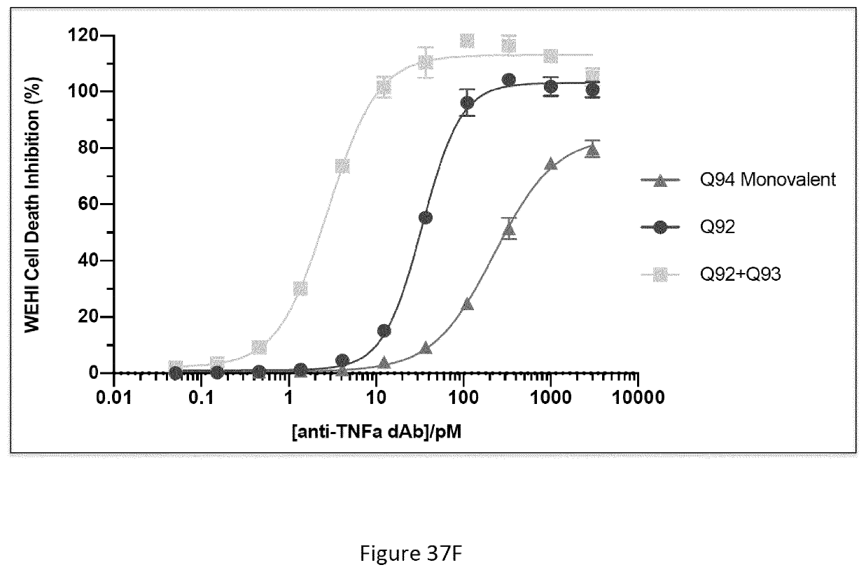

Similar to Example 8, antibody single domains were used to generate monospecific tetravalent and octavalent Quads without IgG Fc (referred to herein by shorhand “non-Ig”). Anti-TNFα dAb VH was linked directly to p53 TD at either the N-terminus to generate tetravalent Quad (Q88 tetravalent) or at both N- and C-terminus to generate octavalent anti-TNFα dAb Quad (Q88 octavalent) as schematically represented in FIG. 22A (embedded in figure as A and B respectively). The original anti-TNFα dAb without the TD domain was also produced for use as a monovalent control (Q88 monovalent). The molecular designs are schematically shown in FIG. 37A, which also highlights the modular design of these Quads.

Following expression in HEK293 cells and Quad protein purification directly from culture supernatant, initial protein analysis was carried out by SDS-PAGE. The multivalent anti-TNFα dAb Quads expressed as highly pure proteins as judged by a single band on the SDS-PAGE gels corresponding to the expected molecular weight (FIG. 37B). To further analyse these multivalent Quads and the effect avidity has on TNFα binding and TNFα neutralization, ELISA binding assay and WEHI cell-based bioassay were performed as described in Examples 9 and 10. Increase in TNFα binding strength can be seen for both tetravalent and octavalent anti-TNFα dAb Quads compared to the anti-TNFα monovalent control in ELISA binding assay (FIG. 37C). Surprisingly, the TNFα binding potential between the octavalent and the tetravalent versions could not be resolved in the ELISA binding assays. Similarly, surprisingly the binding strength for the tetravalent and octavalent anti-TNFα dAb monomeric Ig Quad versions detailed in Example 17 could not be resolved and only a small increase in TNFα binding strength was observed in ELISA binding assay (FIG. 37D). This may be because the binding strength is greatly enhanced and the tetramethylbenzidine-based colorimetric signal is rapidly saturated by these multivalent Quads, and thus the dynamic detection range for this ELISA binding assay is not adequate to differentiate the enhanced binding strength beyond a certain point.

Increase in TNFα binding domain valency was further investigated in WEHI bioassay where the potency of the anti-TNFα Quad molecules to neutralize TNFα-mediated cytotoxicity of WEHI cells were compared. WEHI bioassay was performed as described in Example 10 using both non-Ig and Ig-like anti-TNFα dAb Quads (FIGS. 37E & F). Unlike the ELISA binding assay, in this cell-based bioassay, surprisingly a substantial increase in potency can be seen with increasing anti-TNFα binding domains including major difference in potencies between the tetravalent and octavalent versions. The EC₅₀ values and the fold enhancement of potency of these molecules are summarized in Table 15.

### Example 22: Expression and Functional Potency of Non-Ig Dodeca and Hexadeca Valent Anti-TNFα dAb Quads

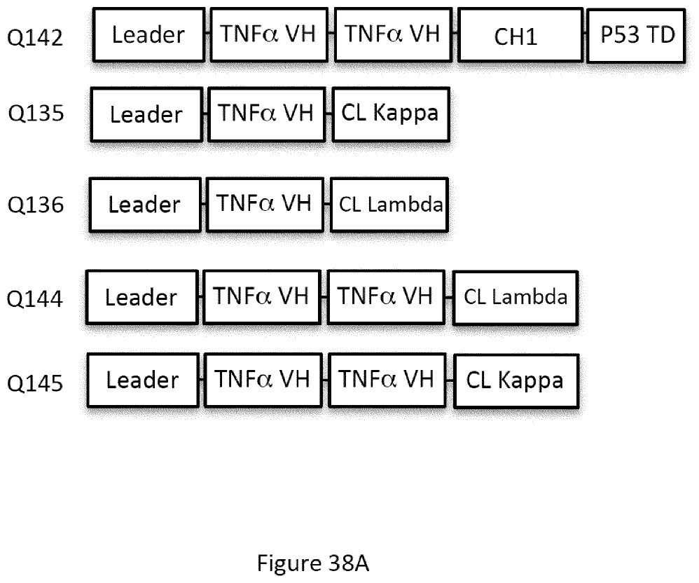

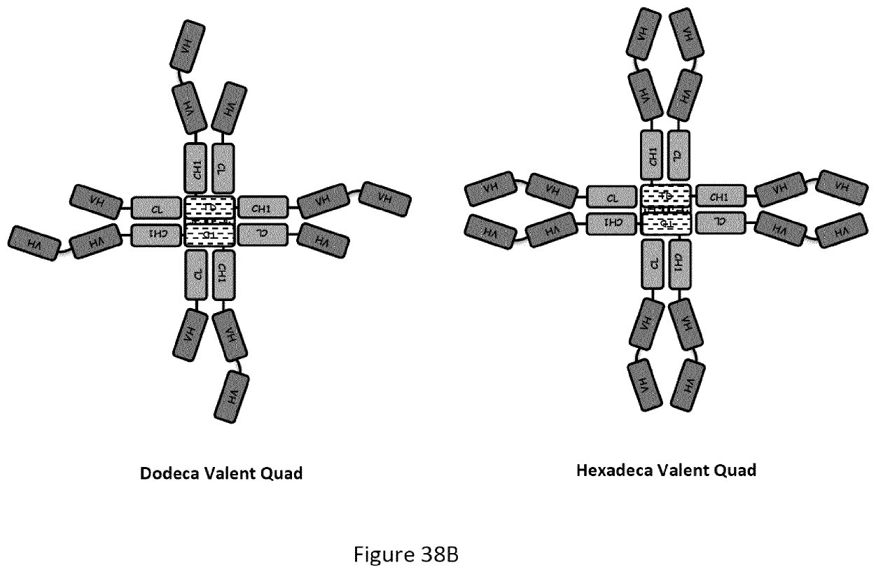

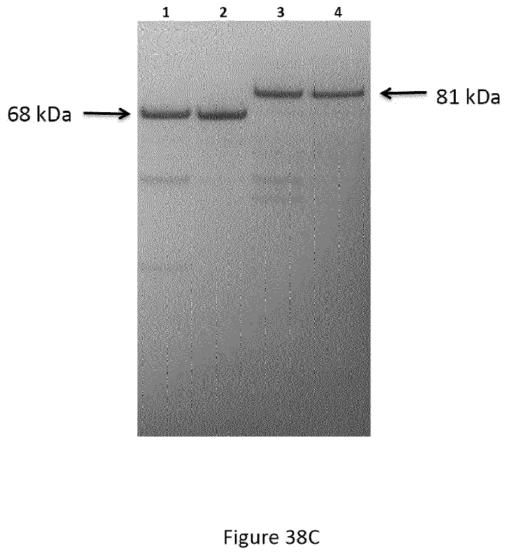

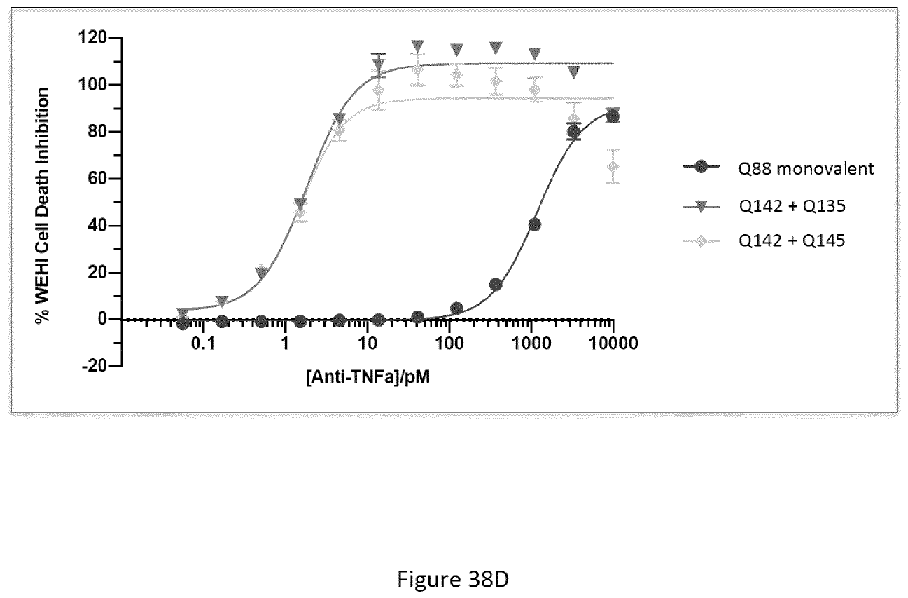

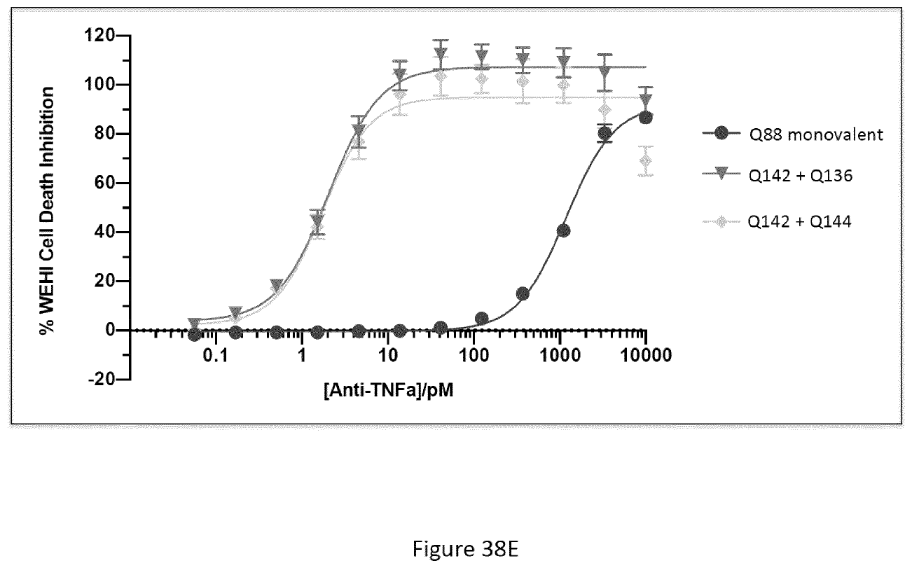

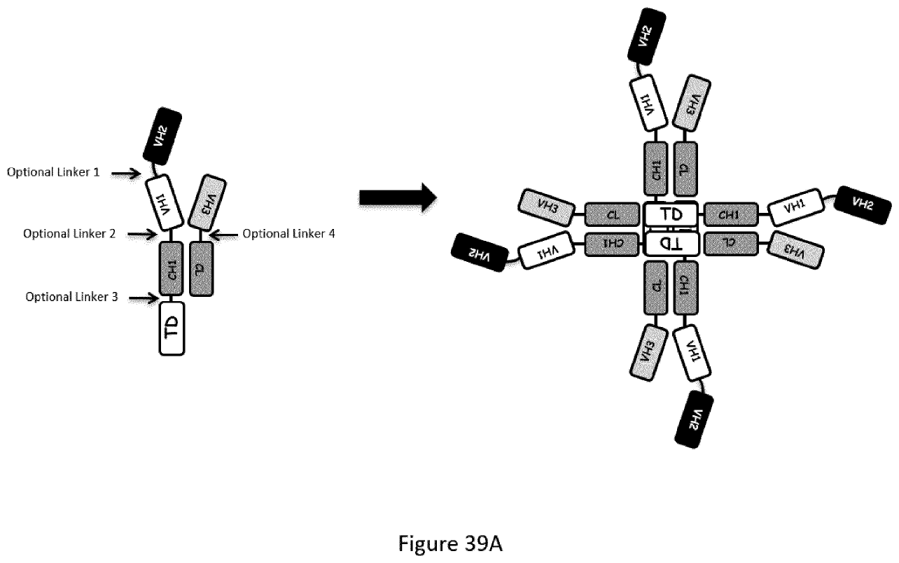

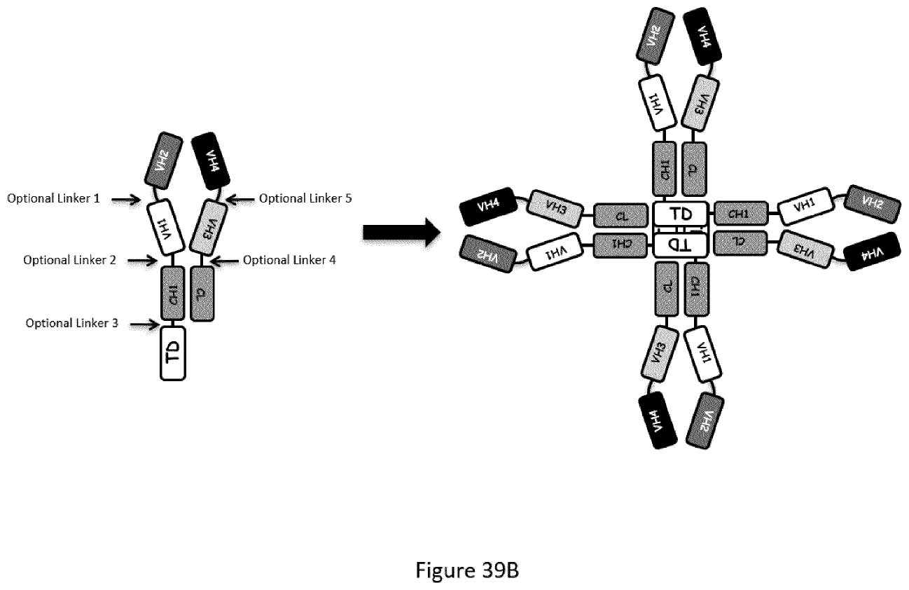

To extend the concept of multivalency further and beyond tetra- and octa-valency, two further formats of anti-TNFα dAb Quads were generated with either 12 (AKA dodeca, 12-valent or 12-mer herein) or 16 (hexadeca, 16-valent or 16-mer herein) anti-TNFα dAb binding domains in a non-Ig format. The modular design and structural arrangements of these Quads can be seen in the schematic illustration in FIGS. 38A&B.

In the dodeca version, the first chain contained tandem anti-TNFα dAbs linked to IgG CH1, which in turn is linked to the TD domain (Q142). The second chain contained a single anti-TNFα dAb linked to either kappa (Q135) or lambda (Q136) light chain constant region. Dodeca-valent Quads were generated by co-expressing the two chains in HEK293 cells where heterodimerisation of the two chains occurred through interaction between CH1 and C-kappa or C-Lambda constant region. The TD domain allowed tetramerisation of these two assembled chains into a tetramer.

For the hexadeca-valent anti-TNFα dAb Quad, the first chain was exactly the same as the dodeca valent format, however, the second chain contained a tandem anti-TNFα dAb linked to either kappa (Q145) or lambda (Q144) light chain constant region. Co-expression of these two chains allowed generation of hexadeca-valent anti-TNFα Quads.

The dodeca- and hexadeca anti-TNFα dAb Quads were expressed in HEK293 cells and the Quad proteins were purified directly from culture supernatant. The purified proteins were analysed by SDS-PAGE (FIG. 38C). The presence of a predominant protein band at the expected molecular weight confirmed these multivalent anti-TNFα dAb Quads can be expressed as highly pure soluble Quad proteins. Furthermore, it confirmed that these multivalent Quads can be generated using either kappa or lambda light chain constant region.

WEHI bioassay was performed using the purified dodeca- and hexadeca anti-TNFα dAb Quads and the TNFα neutralization potency was compared to the monovalent anti-TNFα dAb control (FIGS. 38D&E). The increase anti-TNFα dAb binding domain valency in the dodeca- and hexadeca-valent Quad formats substantially increased the TNFα neutralization potency. The EC₅₀ of these Quads are summarized in Table 16.

Thus, it has been demonstrated that constructs of the invention can surprisingly achieve highly significant increases in antigen binding potency (762-fold in Table 16, for example) and advantageously this can be achieved using different types of binding site (eg, dAb or Fab-like), with or without antibody Fc presence, with possibility of repurposing clinically approved antibodies to retain their tested binding sites and at very high purity levels (almost 100% purity).

### Example 23: Expression Non-Ig Tetravalent Fab Quad

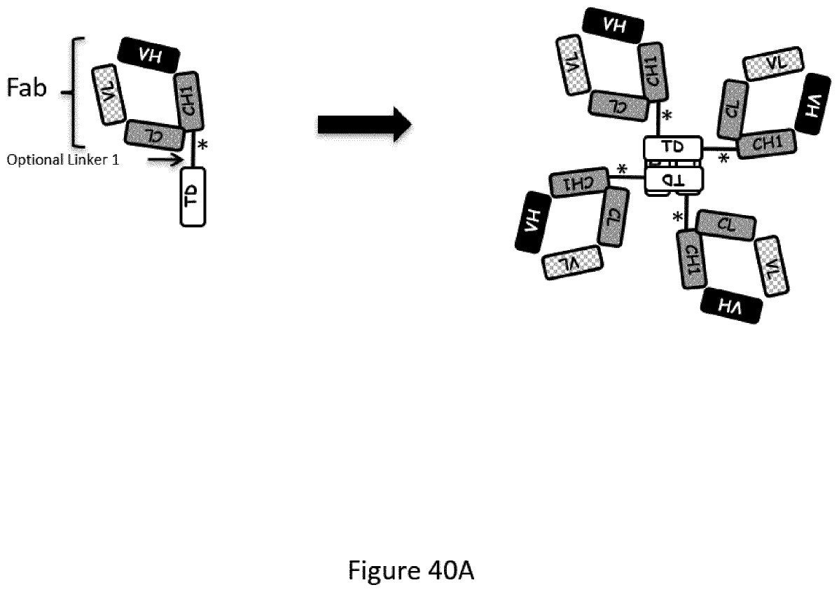

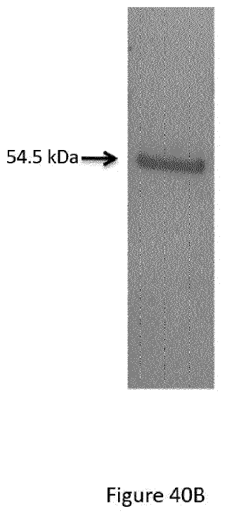

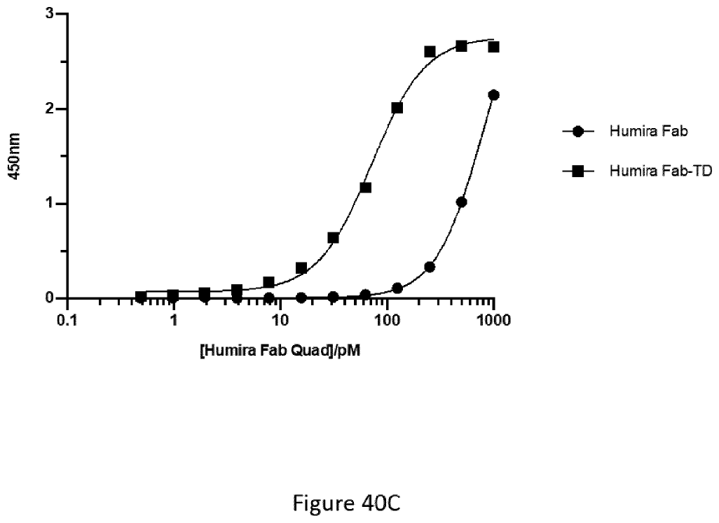

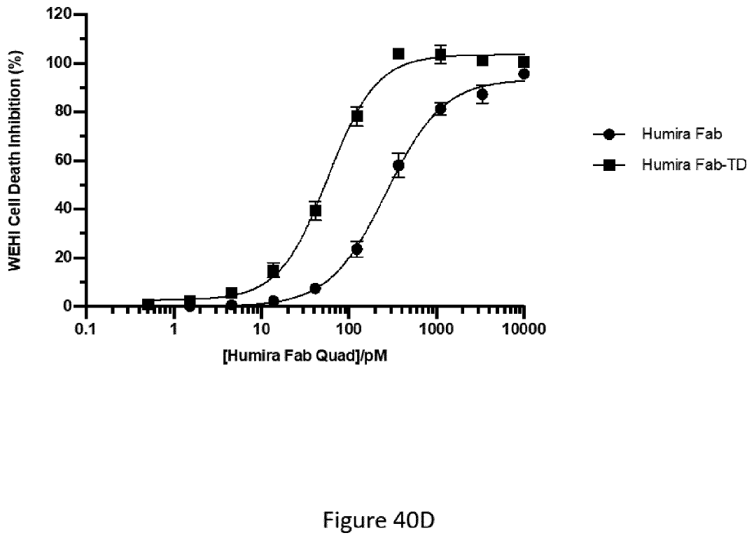

In example 19 production of tetravalent Humira Fab monomeric Ig Quad was exemplified. In this specific example, Humira Fab with intact light chain (LC) and heavy chain (HC) but without Fc region (non-Ig version) was generated. The p53 TD domain was linked at the C-terminus of Humira HC CH1 domain where the hinge region was modified to be devoid of core hinge (ie, upper hinge sequence without core or lower hinge sequence; SEQ ID NO: 183 was used). Co-expression of this modified HC with the native Humira LC, allowed generation of tetravalent Humira Fab-TD with significantly reduced molecular size compared to the Humira Fab monomeric Ig-TD version. A schematic structural representation of Humira Fab-TD is shown in FIG. 40A. “Non-Ig” multimer refers to Quad multimers where the starting monomeric building block does not contain an Fc, as suppose to an “Ig-like” multimer where the starting monomeric building block contain Fc.

The Humira Fab-TD Quad was expressed in HEK293 cells and the Quad protein was purified directly from culture supernatant. The purified protein was analysed by SDS-PAGE (FIG. 40B). The presence of a single protein band at the expected molecular weight confirmed that Humira Fab-TD could be expressed as a soluble protein with high purity (>99% pure).

To further characterize this Quad protein, TNFα binding assay using ELISA and TNFα neutralization potential using WEHI bioassay was performed. As a control Humira Fab was used as a monovalent control. From the ELISA binding assay, it can be seen Humira Fab-TD could bind TNFa with higher binding strength than the Humira Fab monovalent control (FIG. 40C). Similar in the TNFα neutralization bioassay, Humira Fab-TD was able to neutralize TNFα mediated toxicity at higher potency than Humira Fab monovalent control (FIG. 40D). These data confirm that by increasing the valency, the functional affinity is increased resulting in stronger binding and this also enhances the functional potency of Quads compared to the monovalent control.

### Example 24: Octavalent Fab as Non-Ig and Ig-Like Quads

In the examples above, Fabs from antibodies were used to generate tetravalent Quads either as Ig-like (Example 19) or non Ig-like (Example 23). Further iterations of Fab Quads can be made to generate Fabs with octavalent valences either as Ig-like or non Ig-like.

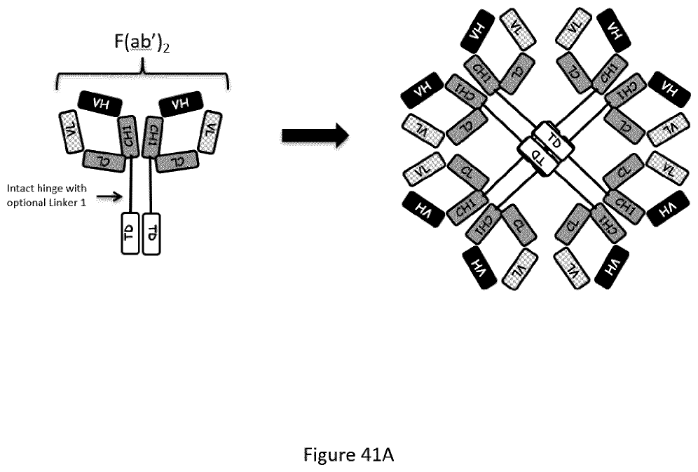

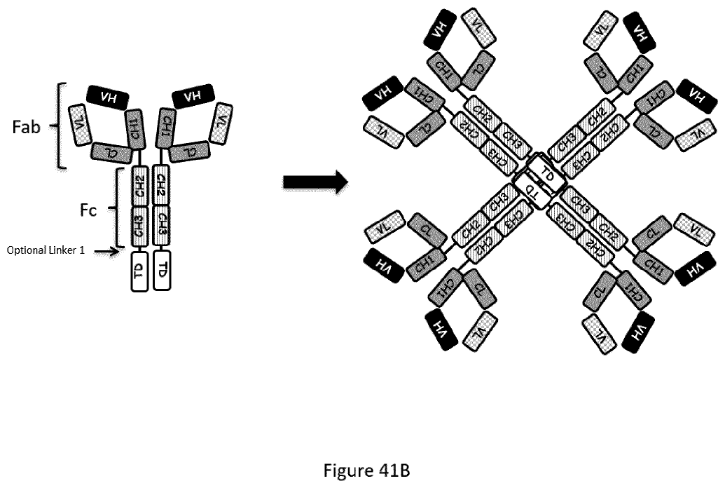

To generate octavalent non Ig-like Fab Quads, a Fab with an intact hinge region will be used where p53 TD domain is linked directly to the hinge region optionally via a flexible peptide linker. The intact core hinge region will allow homodimerization of the Fab-TD when co-expressed with its native LC to generate F(ab′)₂ and as such the monomeric building block will be bivalent. In turn, the TD domain will allow tetramerization of the F(ab′)₂ to generate an octavalent Fab Quad as schematically shown in FIG. 41A.

Similarly, to generate octavalent Ig-like Fab Quads, a TD, eg, a p53 TD domain, will be linked directly to the C-terminus of CH3 domain of an unmodified HC of a predetermined antibody via an optional peptide linker. When this is co-expressed with its native LC from the antibody, the monomeric building block will effectively resemble a fully assembled Ig antibody with p53 TD domains linked to it at the C-terminus of the HC of the assembled antibody. The TD domain in turn will allow tetramerization of the monomeric building blocks to generate an octavalent Fab Ig-like Quad as schematically shown in FIG. 41B. This embodiment is useful to tetramerize any predetermined antibody, such as any predetermined antibody disclosed herein. Thus, for example, a clinically approved antibody can be formatted according to the invention using SAM (eg, TD) mulimerisation to produce a multimer with many more than 2 binding sites for the cognate antigen. For example, the antibody can be bococizumab, alirocumab or evolocumab. In an alternative, instead of using an antibody with SAM (eg, TD), one can use an antigen-binding trap that comprises one or more antigen binding sites and an Fc. For example, one can use aflibercept-SAM as a monomer or aflibercept-TD (such as a p53 or homologue TD as disclosed herein). Thus, a tetramer of aflibercept will be formed by multimerization of the TDs.

Thus, an embodiment provides an antibody comprising a heavy chain, wherein the heavy chain comprises a SAM, eg, a TD (such as a p53 or homologue TD as disclosed herein). The TD may be at the C-terminus of the heavy chain. For example, the heavy chain comprises (in N- to C-terminal direction) a VH, an antibody CH1, a hinge, a CH2, a CH3 and a SAM (eg, a TD). In an example, the heavy chain is paired with a light chain (eg, wherein the light chain comprises (in N- to C-terminal direction) a VL and an antibody CL), wherein a VH and VL comprised by the heavy and light chain pair form an antigen binding site. In an example, the antibody is a 4-chain antibody, such as comprising first and second copies of the heavy and light chain pairs (ie, a first heavy chain paired with a first light chain, a second heavy chain paired with a second light chain, wherein each pair comprises a VH/VL antigen binding site, and wherein the heavy chains are paired together (such as via disulphide bonding in the constant region)). In an example, there is provided a tetramer of such an antibody, wherein the heavy chain of each antibody comprises a TD at its C-terminus and 4 copies of the antibody are tetramerised by the TDs. See, eg, FIG. 41B.

In an example, there is provided an antibody light chain comprising in N- to C-terminal direction) an antibody V domain (eg, a VL, such as a Vκ or a Vλ; or a VH), an antibody CL and a SAM, eg, a TD (such as a p53 or homologue TD as disclosed herein). For example, there is provided a multimer (eg, a tetramer) of such a light chain, optionally wherein the light chain (or each light chain in the multimer) is paired with a second antibody chain comprising (in N- to C-terminal direction) another V domain and a CH1, wherein the V domain and CL of the light chain are paired respectively with the other V domain and CH1.

### REFERENCES

Ali, S. A. et al., 1999. Transferrin Trojan Horses as a Rational Approach for the Biological Delivery of Therapeutic Peptide Domains. Journal of Biological Chemistry, 274(34), pp. 24066-24073.
Barbas et al. (1992) supra
Bentley, G. A. & Mariuzza, R. A., 1996. THE STRUCTURE OF THE T CELL ANTIGEN RECEPTOR. Annual Review of Immunology, 14(1), pp. 563-590.
Binz, H. K. et al., 2003. Designing repeat proteins: well-expressed, soluble and stable proteins from combinatorial libraries of consensus ankyrin repeat proteins. Journal of molecular biology, 332(2), pp. 489-503.
Bixby, K. A. et al., 1999. Zn2+-binding and molecular determinants of tetramerisation in voltage-gated K+ channels. Nature structural biology, 6(1), pp. 38-43.
Borghouts, C., Kunz, C. & Groner, B., 2005. Peptide aptamers: recent developments for cancer therapy. Expert Opinion on Biological Therapy, 5(6), pp. 783-797.
Breitling, F. et al., 1991. A surface expression vector for antibody screening. Gene, 104(2), pp. 147-53.
Burton, D. R. et al., 1991. A large array of human monoclonal antibodies to type 1 human immunodeficiency virus from combinatorial libraries of asymptomatic seropositive individuals. Proceedings of the National Academy of Sciences of the United States of America, 88(22), pp. 10134-7.
Chang, C. N., Landolfi, N. F. & Queen, C., 1991. Expression of antibody Fab domains on bacteriophage surfaces. Potential use for antibody selection. Journal of immunology (Baltimore. Md.: 1950), 147(10), pp. 3610-4.
Chaudhary, V. K. et al., 1990. A rapid method of cloning functional variable-region antibody genes in Escherichia coli as single-chain immunotoxins. Proceedings of the National Academy of Sciences, 87(3), pp. 1066-1070.
Chiswell, D. J. & McCafferty, J., 1992. Phage antibodies: will new “coliclonal” antibodies replace monoclonal antibodies? Trends in biotechnology, 10(3), pp. 80-4.
Clackson, T. et al., 1991. Making antibody fragments using phage display libraries. Nature, 352(6336), pp. 624-8.
Davis, M. M. & Bjorkman, P. J., 1988. T-cell antigen receptor genes and T-cell recognition. Nature, 334(6181), pp. 395-402.
Ding, X.-F. et al., 2008. Characterization and Expression of a Human KCTD1 Gene Containing the BTB Domain, Which Mediates Transcriptional Repression and Homomeric Interactions. DNA and Cell Biology, 27(5), pp. 257-265.
Fujiwara, Y. & Minor, D. L., 2008. X-ray Crystal Structure of a TRPM Assembly Domain Reveals an Antiparallel Four-stranded Coiled-coil. Journal of Molecular Biology, 383(4), pp. 854-870.
Hawkins, R. E. & Winter, G., 1992. Cell selection strategies for making antibodies from variable gene libraries: trapping the memory pool. European journal of immunology, 22(3), pp. 867-70.
Herrin, B. R. & Cooper, M. D., 2010. Alternative adaptive immunity in jawless vertebrates. Journal of immunology (Baltimore. Md.: 1950), 185(3), pp. 1367-74.
Hoogenboom, H. R. et al., 1991. Multi-subunit proteins on the surface of filamentous phage: methodologies for displaying antibody (Fab) heavy and light chains. Nucleic acids research, 19(15), pp. 4133-7.
Hosse, R. J., Rothe, A. & Power, B. E., 2006. A new generation of protein display scaffolds for molecular recognition. Protein science: a publication of the Protein Society, 15(1), pp. 14-27.
Irving, R. A. et al., 2001. Ribosome display and affinity maturation: from antibodies to single V-domains and steps towards cancer therapeutics. Journal of immunological methods, 248(1-2), pp. 31-45.
Jeffrey, P. D., Gorina, S. & Pavletich, N. P., 1995. Crystal structure of the tetramerisation domain of the p53 tumor suppressor at 1.7 angstroms. Science (New York, N.Y.), 267(5203), pp. 1498-502.
Kang, A. S. et al., 1991. Linkage of recognition and replication functions by assembling combinatorial antibody Fab libraries along phage surfaces. Proceedings of the National Academy of Sciences, 88(10), pp. 4363-4366.
Kohl, A. et al., 2003. Designed to be stable: Crystal structure of a consensus ankyrin repeat protein. Proceedings of the National Academy of Sciences, 100(4), pp. 1700-1705.
Lerner, R. A. et al., 1992. Antibodies without immunization. Science (New York, N.Y.), 258(5086), pp. 1313-4.
Liu, Y. et al., 2006. The tetramer structure of the Nervy homology two domain, NHR2, is critical for AML1/ETO's activity. Cancer Cell, 9(4), pp. 249-260.
Lowman, H. B. et al., 1991. Selecting high-affinity binding proteins by monovalent phage display. Biochemistry, 30(45), pp. 10832-8.
Marks et al. (1991) supra
Marks, J. D. et al., 1991. By-passing immunization. Human antibodies from V-gene libraries displayed on phage. Journal of molecular biology, 222(3), pp. 581-97.
Marks, J. D. et al., 1992. Molecular evolution of proteins on filamentous phage. Mimicking the strategy of the immune system. The Journal of biological chemistry, 267(23), pp. 16007-10.
McCafferty et al. (1990) supra
McCafferty, J. et al., 1990. Phage antibodies: filamentous phage displaying antibody variable domains. Nature, 348(6301), pp. 552-4.
Moysey, R., Vuidepot, A.-L. & Boulter, J. M., 2004. Amplification and one-step expression cloning of human T cell receptor genes. Analytical biochemistry, 326(2), pp. 284-6.
Panowski, S. et al., 2014. Site-specific antibody drug conjugates for cancer therapy. mAbs, 6(1), pp. 34-45.
Parker, M. H. et al., 2005. Antibody mimics based on human fibronectin type three domain engineered for thermostability and high-affinity binding to vascular endothelial growth factor receptor two. Protein engineering, design & selection: PEDS, 18(9), pp. 435-44.
Perez, H. L. et al., 2014. Antibody-drug conjugates: Current status and future directions. Drug Discovery Today, 19(7), pp. 869-881.
Reddy Chichili, V. P., Kumar, V. & Sivaraman, J., 2013. Linkers in the structural biology of protein-protein interactions. Protein science: a publication of the Protein Society, 22(2), pp. 153-67.
Shao, C.-Y., Secombes, C. J. & Porter, A. J., 2007. Rapid isolation of IgNAR variable single-domain antibody fragments from a shark synthetic library. Molecular immunology, 44(4), pp. 656-65.
Silverman, J. et al., 2005. Multivalent avimer proteins evolved by exon shuffling of a family of human receptor domains. Nature Biotechnology, 23(12), pp. 1556-1561.
Skerra, A., 2000. Lipocalins as a scaffold. Biochimica et Biophysica Acta (BBA)—Protein Structure and Molecular Enzymology, 1482(1-2), pp. 337-350.
Thie, H. et al., 2009. Multimerization domains for antibody phage display and antibody production. New biotechnology, 26(6), pp. 314-21.
Tonegawa, S., 1988. Somatic generation of immune diversity. Bioscience reports, 8(1), pp. 3-26.
Wälchli, S. et al., 2011. A Practical Approach to T-Cell Receptor Cloning and Expression M. M. Rodrigues, ed. PLoS ONE, 6(11), p. e27930.
Wikman, M. et al., 2004. Selection and characterization of HER2/neu-binding affibody ligands. Protein Engineering Design and Selection, 17(5), pp. 455-462.
Young, P. A., Morrison, S. L. & Timmerman, J. M., 2014. Antibody-cytokine fusion proteins for treatment of cancer: Engineering cytokines for improved efficacy and safety. Seminars in Oncology, 41(5), pp. 623-636.
Zahnd, C. et al., 2007. A designed ankyrin repeat protein evolved to picomolar affinity to Her2. Journal of molecular biology, 369(4), pp. 1015-28.
Zhang, J. et al., 2009. Transient expression and purification of chimeric heavy chain antibodies. Protein Expression and Purification, 65(1), pp. 77-82.
Expert Opinion on Investigational Drugs 16(6) 909-917 (June 2007)
Chapter 7—Non-Antibody Scaffolds from Handbook of Therapeutic Antibodies (2007, edited by Stefan Dubel)
Adams, G. P. et al., 2006. Avidity-mediated enhancement of in vivo tumor targeting by single-chain Fv dimers. Clinical Cancer Research, 12(5), pp. 1599-1605.
Alam, K. et al., 2018. A novel synthetic trivalent single chain variable fragment (tri-scFv) construction platform based on the SpyTag/SpyCatcher protein ligase system, pp. 1-8.
Boruah, B. M. et al., 2013. Single Domain Antibody Multimers Confer Protection against Rabies Infection. PLoS ONE, 8(8).
Brunker, P. et al., 2016. RG7386, a Novel Tetravalent FAP-DR5 Antibody, Effectively Triggers FAP-Dependent, Avidity-Driven DR5 Hyperclustering and Tumor Cell Apoptosis. Molecular Cancer Therapeutics, 15(5), pp. 946-957. Available at: http://mct.aacrjournals.org/cgi/doi/10.1158/1535-7163.MCT-15-0647.
Li, L. et al., 2018. Amplification of CD20 Cross-Linking in Rituximab-Resistant B-Lymphoma Cells Enhances Apoptosis Induction by Drug-Free Macromolecular Therapeutics. ACS Nano, 12(4), pp. 3658-3670.
Mayes, P. A., Hance, K. W. & Hoos, A., 2018. The promise and challenges of immune agonist antibody development in cancer. Nature Reviews Drug Discovery, 17(7), pp. 509-527.
Rudnick, S. I. & Adams, G. P., 2009. Affinity and Avidity in Antibody-Based Tumor Targeting. Cancer Biotherapy & Radiopharmaceuticals, 24(2), pp. 155-161. Available at: http://www.liebertonline.com/doi/abs/10.1089/cbr.2009.0627.
Shen, C. et al., 2019. An IgM antibody targeting the receptor binding site of influenza B blocks viral infection with great breadth and potency. Theranostics, 9(1), pp. 210-231. Available at: http://www.thno.org/v09p0210.htm.
Slaga, D. et al., 2018. Avidity-based binding to HER2 results in selective killing of HER2-overexpressing cells by anti-HER2/CD3. Science Translational Medicine, 10(463).
Wu, A. M. et al., 2001. Multimerization of a chimeric anti-CD20 single-chain Fv-Fc fusion protein is mediated through variable domain exchange. Protein engineering, 14(12), pp. 1025-1033.
Ying, T. et al., 2017. Fc engineering for Developing Therapeutic Bispecific Antibodies and Novel Scaffolds. Article, 8(January), p. 1. Available at: www.frontiersin.org.
Ying, T. et al., 2012. Soluble monomeric IgG1 Fc. Journal of Biological Chemistry, 287(23), pp. 19399-19408.

TABLE 1
p53 Sequences

| SEQ | | | |
|---|---|---|---|
| ID NO: | | AMINO ACID SEQUENCE | NOTES |
| 1 | Human | MEEPQSDPSVEPPLSQETFSDLWKLLPENNVLSPLPSQAMDDLMLSP | Also known as: p53, p53alpha |
| | p53 | DDIEQWFTEDPGPDEAPRPMPEAAPPVPAPAAPTPAAPAPAPSWPLS | This isoform is denoted as the |
| | isoform 1 | SSVPSQKTYQGSYGFRLGFLHSGTAKSVTCTYSPALNKMFCQLAKTC | ‘canonical’ sequence |
| | | PVQLWVDSTPPPGTRVRAMAIYKQSQHMTEVVRRCPHHERCSDSDGL | Tetramerisation sequence in |
| | | APPQHLIRVEGNLRVEYLDDRNTFRHSVVVPYEPPEVGSDCTTIHYN | underline bold (amino acid |
| | | YMCNSSCMGGMNRRPILTIITLEDSSGNLLGRNSFEVRVCACPGRDR | position numbers 325 to 356) |
| | | RTEEENLRKKGEPHHELPPGSTKRALPNNTSSSPQPKKKPLDGEYFT | |
| | | LQIRGRERFEMFRELNEALELKDAQAGKEPGGSRAHSSHLKSKKGQS | |
| | | TRHKKLMFKTEGPDSD | |
| 2 | Human | MEEPQSDPSVEPPLSQETFSDLWKLLPENNVLSPLPSQAMDDLMLSP | Also known as: I9RET, p53beta |
| | p53 | DDIEQWFTEDPGPDEAPRMPEAAPPVAPAPAAPTPAAPAPAPSWPLS | The sequence of this isoform |
| | isoform 2 | SSVPSQKTYQGSYGFRLGFLHSGTAKSVTCTYSPALNKMFCQLAKTC | differs from the canonical |
| | | PVQLWVDSTPPPGTRVRAMAIYKQSQHMTEVVRRCPHHERCSDSDGL | sequence (isoform 1) as |
| | | APPQHLIRVEGNLRVEYLDDRNTFRHSVVVPYEPPEVGSDCTTIHYN | follows: |
| | | YMCNSSCMGGMNRRPILTIITLEDSSGNLLGRNSFEVRVCACPGRDR | 332-341: IRGRERFEMF → |
| | | RTEEENLRKKGEPHHELPPGSTKRALPNNTSSSPQPKKKPLDGEYFT | DQTSFQKENC |
| | | LQDQTSFQKENC | 342-393: Missing. |
| 3 | Human | MEEPQSDPSVEPPLSQETFSDLWKLLPENNVLSPLPSQAMDDLMLSP | Also known as: p53gamma |
| | p53 | DDIEQWFTEDPGPDEAPRMPEAAPPVAPAPAAPTPAAPAPAPSWPLS | The sequence of this isoform |
| | isoform 3 | SSVPSQKTYQGSYGFRLGFLHSGTAKSVTCTYSPALNKMFCQLAKTC | differs from the canonical |
| | | PVQLWVDSTPPPGTRVRAMAIYKQSQHMTEVVRRCPHHERCSDSDGL | sequence as follows: |
| | | APPQHLIRVEGNLRVEYLDDRNTFRHSVVVPYEPPEVGSDCTTIHYN | 332-346: |
| | | YMCNSSCMGGMNRRRILTIITLEDSSGNLLGRNSFEVRVCACPGRDR | IRGRERFEMFRELNE → |
| | | RTEEENLRKKGEPHHELPPGSTKRALPNNTSSSPQPKKKPLDGEYFT | MLLDLRWCYFLINSS |
| | | LQMLLDLRWCYFLINSS | 347-393: Missing |
| 4 | Human | MDDLMLSPDDIEQWFTEDPGPDEAPRMPEAAPPVAPAPAAPTPAAPA | Also known as: Del40-p53, |
| | p53 | PAPSWPLSSSVPSQKTYQGSYGFRLGFLHSGTAKSVTCTYSPALNKM | Del40-p53alpha, p47 |
| | | FCQLAKTCPVQLWVDSTPPPGTRVRAMAIYKQSQHMTEVVRRCPHHE | The sequence of this isoform |
| | | RCSDSDGLAPPQHLIRVEGNLRVEYLDDRNTFRHSVVVPYEPPEVGS | differs from the canonical |
| | | DCTTIHYNYMCNSSCMGGMNRRPILTIITLEDSSGNLLGRNSFEVRV | sequence as follows: |
| | | CACPGRDRRTEEENLRKKGEPHHELPPGSTKRALPNNTSSSPQPKKK | 1-39: Missing. |
| | | PLDGEYFTLQIRGRERFEMFRELNEALELKDAQAGKEPGGSRAHSSH | |
| | | LKSKKGQSTSRHKKLMFKTEGPDSD | |
| 5 | Human | MDDLMLSPDDIEQWFTEDPGPDEAPRMPEAAPPVAPAPAAPTPAAPA | Also known as: Del40-p53beta. |
| | p53 | PAPSWPLSSSVPSQKTYQGSYGFRLGFLHSGTAKSVTCTYSPALNKM | The sequence of this isoform |
| | isoform 5 | FCQLAKTCPVQLWVDSTPPPGTRVRAMAIYKQSQHMTEVVRRCPHHE | differs from the canonical |
| | | RCSDSDGLAPPQHLIRVEGNLRVEYLDDRNTFRHSVVVPYEPPEVGS | sequence as follows: |
| | | DCTTIHYNYMCNSSCMGGMNRRPILTIITLEDSSGNLLGRNSFEVRV | 1-39: Missing. |
| | | CACPGRDRRTEEENLRKKGEPHHELPPGSTKRALPNNTSSSPQPKKK | 332-341: IRGRERFEMF → |
| | | PLDGEYFTLQDQTSFQKENC | DQTSFQKENC |
| | | | 342-393: Missing. |
| 6 | Human | MDDLMLSPDDIEQWFTEDPGPDEAPRMPEAAPPVAPAPAAPTPAAPA | Also known as: Del40- |
| | p53 | PAPSWPLSSSVPSQKTYQGSYGFRLGFLHSGTAKSVTCTYSPALNKM | p53gamma. The sequence of |
| | isoform 6 | FCQLAKTCPVQLWVDSTPPPGTRVRAMAIYKQSQHMTEVVRRCPHHE | this isoform differs from the |
| | | RCSDSDGLAPPQHLIRVEGNLRVEYLDDRNTFRHSVVVPYEPPEVGS | canonical sequence as follows: |
| | | DCTTIHYNYMCNSSCMGGMNRRPILTIITLEDSSGNLLGRNSFEVRV | 1-39: Missing. |
| | | CACPGRDRRTEEENLRKKGEPHHELPPGSTKRALPNNTSSSPQPKKK | 332-346: |
| | | PLDGEYFTLQMLLDLRWCYFLINSS | IRGRERFEMFRELNE → |
| | | | MLLDLRWCYFLINSS |
| | | | 347-393: Missing. |
| 7 | Human | MFCQLAKTCPVQLWVDSTPPPGTRVRAMAIYKQSQHMTEVVRRCPHH | Also known as: Del133-p53, |
| | p53 | ERCSDSDGLAPPQHLIRVEGNLRVEYLDDRNTFRHSVVVPYEPPEVG | Del133-p53alpha. The |
| | isoform 7 | SDCTTIHYNYMCNSSCMGGMNRRPILTIITLEDSSGNLLGRNSFEVR | sequence of this isoform |
| | | VCACPGRDRRTEEENLRKKGEPHHELPPGSTKRALPNNTSSSPQPKK | differs from the canonical |
| | | KPLDGEYFTLQIRGRERFEMFRELNEALELKDAQAGKEPGGSRAHSS | sequence as follows: |
| | | HLKSKKGQSTSRHKKLMFKTEGPDSD | 1-132: Missing. |
| 8 | Human | MFCQLAKTCPVQLWVDSTPPPGTRVRAMAIYKQSQHMTEVVRRCPHH | Also known as: Del133- |
| | p53 | ERCSDSDGLAPPQHLIRVEGNLRVEYLDDRNTFRHSVVVPYEPPEVG | p53beta. The sequence of this |
| | isoform 8 | SDCTTIHYNYMCNSSCMGGMNRRPILTIITLEDSSGNLLGRNSFEVR | isoform differs from the |
| | | VCACPGRDRRTEEENLRKKGEPHHELPPGSTKRALPNNTSSSPQPKK | canonical sequence as |
| | | KPLDGEYFTLQDQTSFQKENC | follows: |
| | | | 1-132: Missing. |
| | | | 332-341: IRGRERFEMF → |
| | | | DQTSFQKENC |
| | | | 342-393: Missing. |
| 9 | Human | MFCQLAKTCPVQLWVDSTPPPGTRVRAMAIYKQSQHMTEVVRRCPHH | Also known as: Del133- |
| | p53 | ERCSDSDGLAPPQHLIRVEGNLRVEYLDDRNTFRHSVVVPYEPPEVG | p53gamma. The sequence of |
| | isoform 9 | SDCTTIHYNYMCNSSCMGGMNRRPILTIITLEDSSGNLLGRNSFEVR | this isoform differs from the |
| | | VCACPGRDRRTEEENLRKKGEPHHELPPGSTKRALPNNTSSSPQPKK | canonical sequence as follows: |
| | | KPLDGEYFTLQMLLDLRWCYFLINSS | 1-132: Missing. |
| | | | 332-346: |
| | | | IRGRERFEMFRELNE → |
| | | | MLLDLRWCYFLINSS |
| | | | 347-393: Missing. |
| 10 | A human | GEYFTLQIRGRERFEMFRELNEALELKDAQAG | |
| | p53-TD | | |
| 11 | A human | RSPDDELLYLPVRGRETYEMLLKIKESLELMQYLPQHTIETYRQQQQ | |
| | p63-TD | QQH | |
| 12 | A human | KKRRHGDEDTYYLQVRGRENFEILMKLKESLELMELVPQPLV | |
| | p73-TD | | |

TABLE 2
Example Human Proteins Comprising a Tetramerisation Domain

| Protein | | | | |
|---|---|---|---|---|
| Number | Uniprot Entry | Entry name | Protein names | Gene names |
| 1 | P04637 | P53_HUMAN | Cellular tumor antigen p53 (Antigen NY-CO-13) | TP53 P53 |
| | | | (Phosphoprotein p53) (Tumor suppressor p53) | |
| 2 | Q719H9 | KCTD1_HUMAN | BTB/POZ domain-containing protein KCTD1 (Potassium | KCTD1 C18orf5 |
| | | | channel tetramerisation domain-containing protein 1) | |
| 3 | P51787 | KCNQ1_HUMAN | Potassium voltage-gated channel subfamily KQT member 1 | KCNQ1 KCNA8 |
| | | | (IKs producing slow voltage-gated potassium channel subunit | KCNA9 KVLQT1 |
| | | | alpha KvLQT1) (KQT-like 1) (Voltage-gated potassium | |
| | | | channel subunit Kv7.1) | |
| 4 | Q06455 | MTG8_HUMAN | Protein CBFA2T1 (Cyclin-D-related protein) (Eight twenty | RUNX1T1 |
| | | | one protein) (Protein ETO) (Protein MTG8) (Zinc finger | AML1T1 CBFA2T1 |
| | | | MYND domain-containing protein 2) | CDR ETO MTG8 |
| | | | | ZMYND2 |
| 5 | Q9H3F6 | BACD3_HUMAN | BTB/POZ domain-containing adapter for CUL3-mediated | KCTD10 ULR061 |
| | | | RhoA degradation protein 3 (hBACURD3) (BTB/POZ | MSTP028 |
| | | | domain-containing protein KCTD10) (Potassium channel | |
| | | | tetramerisation domain-containing protein 10) | |
| 6 | Q12809 | KCNH2_HUMAN | Potassium voltage-gated channel subfamily H member 2 (Eag | KCNH2 ERG ERG1 |
| | | | homolog) (Ether-a-go-go-related gene potassium channel 1) | HERG |
| | | | (ERG-1) (Eag-related protein 1) (Ether-a-go-go-related | |
| | | | protein 1) (H-ERG) (hERG-1) (hERG1) (Voltage-gated | |
| | | | potassium channel subunit Kv11.1) | |
| 7 | Q96SI1 | KCD15_HUMAN | BTB/POZ domain-containing protein KCTD15 (Potassium | KCTD15 |
| | | | channel tetramerisation domain-containing protein 15) | |
| 8 | P02766 | TTHY_HUMAN | Transthyretin (ATTR) (Prealbumin) (TBPA) | TTR PALB |
| 9 | Q14681 | KCTD2_HUMAN | BTB/POZ domain-containing protein KCTD2 (Potassium | KCTD2 KIAA0176 |
| | | | channel tetramerisation domain-containing protein 2) | |
| 10 | Q7Z5Y7 | KCD20_HUMAN | BTB/POZ domain-containing protein KCTD20 (Potassium | KCTD20 C6orf69 |
| | | | channel tetramerisation domain containing 20) | |
| 11 | P50552 | VASP_HUMAN | Vasodilator-stimulated phosphoprotein (VASP) | VASP |
| 12 | Q68DU8 | KCD16_HUMAN | BTB/POZ domain-containing protein KCTD16 (Potassium | KCTD16 |
| | | | channel tetramerisation domain-containing protein 16) | KIAA1317 |
| 13 | Q09470 | KCNA1_HUMAN | Potassium voltage-gated channel subfamily A member 1 | KCNA1 |
| | | | (Voltage-gated K(+) channel HuK1) (Voltage-gated | |
| | | | potassium channel HBK1) (Voltage-gated potassium channel | |
| | | | subunit Kv1.1) | |
| 14 | P13501 | CCL5_HUMAN | C-C motif chemokine 5 (EoCP) (Eosinophil chemotactic | CCL5 D17S136E |
| | | | cytokine) (SIS-delta) (Small-inducible cytokine A5) (T cell- | SCYA5 |
| | | | specific protein P228) (TCP228) (T-cell-specific protein | |
| | | | RANTES) [Cleaved into: RANTES(3-68); RANTES(4-68)] | |
| 15 | P08069 | IGF1R_HUMAN | Insulin-like growth factor 1 receptor (EC 2.7.10.1) (Insulin- | IGF1R |
| | | | like growth factor I receptor) (IGF-I receptor) (CD antigen | |
| | | | CD221) [Cleaved into: Insulin-like growth factor 1 receptor | |
| | | | alpha chain; Insulin-like growth factor 1 receptor beta chain] | |
| 16 | Q14003 | KCNC3_HUMAN | Potassium voltage-gated channel subfamily C member 3 | KCNC3 |
| | | | (KSHIIID) (Voltage-gated potassium channel subunit Kv3.3) | |
| 17 | O15350 | P73_HUMAN | Tumor protein p73 (p53-like transcription factor) (p53-related | TP73 P73 |
| | | | protein) | |
| 18 | Q12791 | KCMA1_HUMAN | Calcium-activated potassium channel subunit alpha-1 (BK | KCNMA1 KCNMA |
| | | | channel) (BKCA alpha) (Calcium-activated potassium | SLO |
| | | | channel, subfamily M subunit alpha-1) (K(VCA)alpha) | |
| | | | (KCa1.1) (Maxi K channel) (MaxiK) (Slo-alpha) (Slo1) | |
| | | | (Slowpoke homolog) (Slo homolog) (hSlo) | |
| 19 | P42261 | GRIA1_HUMAN | Glutamate receptor 1 (GluR-1) (AMPA-selective glutamate | GRIA1 GLUH1 |
| | | | receptor 1) (GluR-A) (GluR-K1) (Glutamate receptor | GLUR1 |
| | | | ionotropic, AMPA 1) (GluA1) | |
| 20 | P22303 | ACES_HUMAN | Acetylcholinesterase (AChE) (EC 3.1.1.7) | ACHE |
| 21 | P04040 | CATA_HUMAN | Catalase (EC 1.11.1.6) | CAT |
| 22 | Q9H8Y8 | GORS2_HUMAN | Golgi reassembly-stacking protein 2 (GRS2) (Golgi | GORASP2 |
| | | | phosphoprotein 6) (GOLPH6) (Golgi reassembly-stacking | GOLPH6 |
| | | | protein of 55 kDa) (GRASP55) (p59) | |
| 23 | P34897 | GLYM_HUMAN | Serine hydroxymethyltransferase, mitochondrial (SHMT) | SHMT2 |
| | | | (EC 2.1.2.1) (Glycine hydroxymethyltransferase) (Serine | |
| | | | methylase) | |
| 24 | P13760 | 2B14_HUMAN | HLA class II histocompatibility antigen, DRB1-4 beta chain | HLA-DRB1 |
| | | | (MHC class II antigen DRB1*4) (DR-4) (DR4) | |
| 25 | P04229 | 2B11_HUMAN | HLA class II histocompatibility antigen, DRB1-1 beta chain | HLA-DRB1 |
| | | | (MHC class II antigen DRB1*1) (DR-1) (DR1) | |
| 26 | P35914 | HMGCL_HUMAN | Hydroxymethylglutaryl-CoA lyase, mitochondrial (HL) | HMGCL |
| | | | (HMG-CoA lyase) (EC 4.1.3.4) (3-hydroxy-3- | |
| | | | methylglutarate-CoA lyase) | |
| 27 | Q29974 | 2B1G_HUMAN | HLA class II histocompatibility antigen, DRB1-16 beta chain | HLA-DRB1 |
| | | | (MHC class II antigen DRB1*16) (DR-16) (DR16) | |
| 28 | Q9TQE0 | 2B19_HUMAN | HLA class II histocompatibility antigen, DRB1-9 beta chain | HLA-DRB1 |
| | | | (MHC class II antigen DRB1*9) (DR-9) (DR9) | |
| 29 | Q9UDR5 | AASS_HUMAN | Alpha-aminoadipic semialdehyde synthase, mitochondrial | AASS |
| | | | (LKR/SDH) [Includes: Lysine ketoglutarate reductase (LKR) | |
| | | | (LOR) (EC 1.5.1.8); Saccharopine dehydrogenase (SDH) (EC | |
| | | | 1.5.1.9)] | |
| 30 | P13761 | 2B17_HUMAN | HLA class II histocompatibility antigen, DRB1-7 beta chain | HLA-DRB1 |
| | | | (MHC class II antigen DRB1*7) (DR-7) (DR7) | |
| 31 | P49450 | CENPA_HUMAN | Histone H3-like centromeric protein A (Centromere | CENPA |
| | | | autoantigen A) (Centromere protein A) (CENP-A) | |
| 32 | Q9Y2W7 | CSEN_HUMAN | Calsenilin (A-type potassium channel modulatory protein 3) | KCNIP3 CSEN |
| | | | (DRE-antagonist modulator) (DREAM) (Kv channel- | DREAM KCHIP3 |
| | | | interacting protein 3) (KChIP3) | |
| 33 | Q16630 | CPSF6_HUMAN | Cleavage and polyadenylation specificity factor subunit 6 | CPSF6 CFIM68 |
| | | | (Cleavage and polyadenylation specificity factor 68 kDa | |
| | | | subunit) (CFIm68) (CPSF 68 kDa subunit) (Pre-mRNA | |
| | | | cleavage factor Im 68 kDa subunit) (Protein HPBRII-4/7) | |
| 34 | O43809 | CPSF5_HUMAN | Cleavage and polyadenylation specificity factor subunit 5 | NUDT21 CFIM25 |
| | | | (Cleavage and polyadenylation specificity factor 25 kDa | CPSF25 CPSF5 |
| | | | subunit) (CFIm25) (CPSF 25 kDa subunit) (Nucleoside | |
| | | | diphosphate-linked moiety X motif 21) (Nudix motif 21) | |
| | | | (Pre-mRNA cleavage factor Im 25 kDa subunit) | |
| 35 | Q8N684 | CPSF7_HUMAN | Cleavage and polyadenylation specificity factor subunit 7 | CPSF7 |
| | | | (Cleavage and polyadenylation specificity factor 59 kDa | |
| | | | subunit) (CFIm59) (CPSF 59 kDa subunit) (Pre-mRNA | |
| | | | cleavage factor Im 59 kDa subunit) | |
| 36 | Q14999 | CUL7_HUMAN | Cullin-7 (CUL-7) | CUL7 KIAA0076 |
| 37 | Q9U108 | EVL_HUMAN | Ena/VASP-like protein (Ena/vasodilator-stimulated | EVL RNB6 |
| | | | phosphoprotein-like) | |
| 38 | Q05193 | DYN1_HUMAN | Dynamin-1 (EC 3.6.5.5) | DNM1 DNM |
| 39 | Q8N8S7 | ENAH_HUMAN | Protein enabled homolog | ENAH MENA |
| 40 | Q96PP8 | GBP5_HUMAN | Guanylate-binding protein 5 (EC 3.6.5.-) (GBP-TA antigen) | GBP5 |
| | | | (GTP-binding protein 5) (GBP-5) (Guanine nucleotide- | UNQ2427/PR04987 |
| | | | binding protein 5) | |
| 41 | Q92947 | GCDH_HUMAN | Glutaryl-CoA dehydrogenase, mitochondrial (GCD) (EC | GCDH |
| | | | 1.3.8.6) | |
| 42 | Q13614 | MTMR2_HUMAN | Myotubularin-related protein 2 (Phosphatidylinositol-3,5- | MTMR2 KIAA1073 |
| | | | bisphosphate 3-phosphatase) (EC 3.1.3.95) | |
| | | | (Phosphatidylinositol-3-phosphate phosphatase) (EC | |
| | | | 3.1.3.64) | |
| 43 | Q99784 | NOE1_HUMAN | Noelin (Neuronal olfactomedin-related ER localized protein) | OLFM1 NOE1 |
| | | | (Olfactomedin-1) | NOEL1 |
| 44 | P50542 | PEX5_HUMAN | Peroxisomal targeting signal 1 receptor (PTS1 receptor) | PEX5 PXR1 |
| | | | (PTS1R) (PTS1-BP) (Peroxin-5) (Peroxisomal C-terminal | |
| | | | targeting signal import receptor) (Peroxisome receptor 1) | |
| 45 | P42262 | GRIA2_HUMAN | Glutamate receptor 2 (GluR-2) (AMPA-selective glutamate | GRIA2 GLUR2 |
| | | | receptor 2) (GluR-B) (GluR-K2) (Glutamate receptor | |
| | | | ionotropic, AMPA 2) (GluA2) | |
| 46 | P48058 | GRIA4_HUMAN | Glutamate receptor 4 (GluR-4) (GluR4) (AMPA-selective | GRIA4 GLUR4 |
| | | | glutamate receptor 4) (GluR-D) (Glutamate receptor | |
| | | | ionotropic, AMPA 4) (GluA4) | |
| 47 | P42263 | GRIA3_HUMAN | Glutamate receptor 3 (GluR-3) (AMPA-selective glutamate | GRIA3 GLUR3 |
| | | | receptor 3) (GluR-C) (GluR-K3) (Glutamate receptor | GLURC |
| | | | ionotropic, AMPA 3) (GluA3) | |
| 48 | O60741 | HCN1_HUMAN | Potassium/sodium hyperpolarization-activated cyclic | HCN1 BCNG1 |
| | | | nucleotide-gated channel 1 (Brain cyclic nucleotide-gated | |
| | | | channel 1) (BCNG-1) | |
| 49 | Q9UL51 | HCN2_HUMAN | Potassium/sodium hyperpolarization-activated cyclic | HCN2 BCNG2 |
| | | | nucleotide-gated channel 2 (Brain cyclic nucleotide-gated | |
| | | | channel 2) (BCNG-2) | |
| 50 | Q9Y3Q4 | HCN4_HUMAN | Potassium/sodium hyperpolarization-activated cyclic | HCN4 |
| | | | nucleotide-gated channel 4 | |
| 51 | P04035 | HMDH_HUMAN | 3-hydroxy-3-methylglutaryl-coenzyme A reductase (HMG- | HMGCR |
| | | | CoA reductase) (EC 1.1.1.34) | |
| 52 | Q8NCD3 | HJURP_HUMAN | Holliday junction recognition protein (14-3-3-associated | HJURP FAKTS |
| | | | AKT substrate) (Fetal liver-expressing gene 1 protein) (Up- | FLEG1 URLC9 |
| | | | regulated in lung cancer 9) | |
| 53 | Q9NZV8 | KCND2_HUMAN | Potassium voltage-gated channel subfamily D member 2 | KCND2 KIAA1044 |
| | | | (Voltage-gated potassium channel subunit Kv4.2) | |
| 54 | P48547 | KCNC1_HUMAN | Potassium voltage-gated channel subfamily C member 1 | KCNC1 |
| | | | (NGK2) (Voltage-gated potassium channel subunit Kv3.1) | |
| | | | (Voltage-gated potassium channel subunit Kv4) | |
| 55 | Q96CX2 | KCD12_HUMAN | BTB/POZ domain-containing protein KCTD12 (Pfetin) | KCTD12 C13orf2 |
| | | | (Predominantly fetal expressed T1 domain) | KIAA1778 PFET1 |
| 56 | P16389 | KCNA2_HUMAN | Potassium voltage-gated channel subfamily A member 2 | KCNA2 |
| | | | (NGK1) (Voltage-gated K(+) channel HuKIV) (Voltage- | |
| | | | gated potassium channel HBK5) (Voltage-gated potassium | |
| | | | channel subunit Kv1.2) | |
| 57 | P56696 | KCNQ4_HUMAN | Potassium voltage-gated channel subfamily KQT member 4 | KCNQ4 |
| | | | (KQT-like 4) (Potassium channel subunit alpha KvLQT4) | |
| | | | (Voltage-gated potassium channel subunit Kv7.4) | |
| 58 | Q9NXV2 | KCTD5_HUMAN | BTB/POZ domain-containing protein KCTD5 | KCTD5 |
| 59 | Q14721 | KCNB1_HUMAN | Potassium voltage-gated channel subfamily B member 1 | KCNB1 |
| | | | (Delayed rectifier potassium channel 1) (DRK1) (h-DRK1) | |
| | | | (Voltage-gated potassium channel subunit Kv2.1) | |
| 60 | Q86WG5 | MTMRD_HUMAN | Myotubularin-related protein 13 (SET-binding factor 2) | SBF2 CMT4B2 |
| | | | | KIAA1766 |
| | | | | MTMR13 |
| 61 | Q15070 | OXAlL_HUMAN | Mitochondrial inner membrane protein OXA1L (Hsa) | OXA1L |
| | | | (OXA1Hs) (Oxidase assembly 1-like protein) (OXA1-like | |
| | | | protein) | |
| 62 | P11498 | PYC_HUMAN | Pyruvate carboxylase, mitochondrial (EC 6.4.1.1) (Pyruvic | PC |
| | | | carboxylase) (PCB) | |
| 63 | P33764 | S10A3_HUMAN | Protein S100-A3 (Protein S-100E) (S100 calcium-binding | S100A3 S100E |
| | | | protein A3) | |
| 64 | P58743 | S26A5_HUMAN | Prestin (Solute carrier family 26 member 5) | SLC26A5 PRES |
| 65 | Q9UIL1 | SCOC_HUMAN | Short coiled-coil protein | SCOC SCOCO |
| | | | | HRIHFB2072 |
| 66 | P02549 | SPTA1_HUMAN | Spectrin alpha chain, erythrocytic 1 (Erythroid alpha- | SPTA1 SPTA |
| | | | spectrin) | |
| 67 | Q96QT4 | TRPM7_HUMAN | Transient receptor potential cation channel subfamily M | TRPM7 CHAK1 |
| | | | member 7 (EC 2.7.11.1) (Channel-kinase 1) (Long transient | LTRPC7 |
| | | | receptor potential channel 7) (LTrpC-7) (LTrpC7) | |
| 68 | Q9HCF6 | TRPM3_HUMAN | Transient receptor potential cation channel subfamily M | TRPM3 KIAA1616 |
| | | | member 3 (Long transient receptor potential channel 3) | LTRPC3 |
| | | | (LTrpC-3) (LTrpC3) (Melastatin-2) (MLSN2) | |
| 69 | Q7Z4N2 | TRPM1_HUMAN | Transient receptor potential cation channel subfamily M | TRPM1 LTRPC1 |
| | | | member 1 (Long transient receptor potential channel 1) | MLSN MLSN1 |
| | | | (LTrp C1) (Melastatin-1) | |
| 70 | Q9NQA5 | TRPV5_HUMAN | Transient receptor potential cation channel subfamily V | TRPV5 ECAC1 |
| | | | member 5 (TrpV5) (Calcium transport protein 2) (CaT2) | |
| | | | (Epithelial calcium channel 1) (ECaC) (ECaC1) (Osm-9-like | |
| | | | TRP channel 3) (OTRPC3) | |
| 71 | Q9BX84 | TRPM6_HUMAN | Transient receptor potential cation channel subfamily M | TRPM6 CHAK2 |
| | | | member 6 (EC 2.7.11.1) (Channel kinase 2) (Melastatin- | |
| | | | related TRP cation channel 6) | |
| 72 | P49638 | TTPA_HUMAN | Alpha-tocopherol transfer protein (Alpha-TTP) | TTPA TPP1 |
| 73 | Q8NBZ7 | UXS1_HUMAN | UDP-glucuronic acid decarboxylase 1 (EC 4.1.1.35) (UDP- | UXS1 |
| | | | glucuronate decarboxylase 1) (UGD) (UXS-1) | UNQ2538/PRO6079 |
| 74 | Q13426 | XRCC4_HUMAN | DNA repair protein XRCC4 (X-ray repair cross- | XRCC4 |
| | | | complementing protein 4) | |
| 75 | A0A0A6YY98 | A0A0A6YY98_HUMAN | Transient receptor potential cation channel subfamily V | TRPV5 |
| | | | member 5 | |
| 76 | H0YLN8 | H0YLN8_HUMAN | Transient receptor potential cation channel subfamily M | TRPM7 hCG_39859 |
| | | | member 7 (Transient receptor potential cation channel, | |
| | | | subfamily M, member 7, isoform CRA_c) | |
| 77 | A0A0C4DFW9 | A0A0C4DFW9_HUMAN | Cellular tumor antigen p53 | TP73 hCG_19088 |
| 78 | G5E9G1 | G5E9G1_HUMAN | Transient receptor potential cation channel subfamily M | TRPM3 |
| | | | member 3 (Transient receptor potential cation channel, | hCG_2042991 |
| | | | subfamily M, member 3, isoform CRA_a) | |
| 79 | H7BYP1 | H7BYP1_HUMAN | Transient receptor potential cation channel subfamily M | TRPM3 |
| | | | member 3 (Transient receptor potential cation channel, | hCG_2042991 |
| | | | subfamily M, member 3, isoform CRA_c) | |
| 80 | A0A024R4C3 | A0A024R4C3_HUMAN | Tumor protein p73, isoform CRA_a | TP73 hCG_19088 |
| 81 | A0A0S2Z4N5 | A0A0S2Z4N5_HUMAN | Tumor protein p63 isoform 1 (Tumor protein p73-like, | TP63 TP73L |
| | | | isoform CRA_a) (Fragment) | hCG_16028 |
| 82 | A0A024R5V1 | A0A024R5V1_HUMAN | Transient receptor potential cation channel, subfamily M, | TRPM7 hCG_39859 |
| | | | member 7, isoform CRA_a | |
| 83 | X5D8S6 | X5D8S6_HUMAN | Adenylosuccinate lyase (ASL) (EC 4.3.2.2) | ADSL hCG_40060 |
| | | | (Adenylosuccinase) (Fragment) | |
| 84 | C9D7D0 | C9D7D0_HUMAN | Cellular tumor antigen p53 | TP63 |
| 85 | K7PPA8 | K7PPA8_HUMAN | Cellular tumor antigen p53 | TP53 |
| 86 | A2A3F4 | A2A3F4_HUMAN | Transient receptor potential cation channel subfamily M | TRPM3 |
| | | | member 3 | |
| 87 | Q2XSC7 | Q2XSC7_HUMAN | Cellular tumor antigen p53 | TP53 |
| 88 | A0A024R209 | A0A024R209_HUMAN | Transient receptor potential cation channel, subfamily M, | TRPM1 hCG_37570 |
| | | | member 1, isoform CRA_a | |
| 89 | Q1MSX0 | Q1MSX0_HUMAN | Cellular tumor antigen p53 (Fragment) | TP53 |
| 90 | A0A0G2JN34 | A0A0G2JN34_HUMAN | Transient receptor potential cation channel subfamily M | TRPM1 |
| | | | member 1 | |
| 91 | H6U5S3 | H6U5S3_HUMAN | Cellular tumor antigen p53 (Fragment) | |
| 92 | H2EHT1 | H2EHT1_HUMAN | Cellular tumor antigen p53 | TP53 |
| 93 | H6U5S2 | H6U5S2_HUMAN | Cellular tumor antigen p53 (Fragment) | |
| 94 | B4DNI2 | B4DNI2_HUMAN | Cellular tumor antigen p53 | |
| 95 | C9D7C9 | C9D7C9_HUMAN | Cellular tumor antigen p53 | TP63 |
| 96 | B6E4X6 | B6E4X6_HUMAN | Cellular tumor antigen p53 | |
| 97 | A0A087WZU8 | A0A087WZU8_HUMAN | Cellular tumor antigen p53 | TP53 |
| 98 | K7PPU4 | K7PPU4_HUMAN | Cellular tumor antigen p53 | TP53 |
| 99 | A0A0U1RQC9 | A0A0U1RQC9_HUMAN | Cellular tumor antigen p53 | TP53 |
| 100 | Q5U0E4 | Q5U0E4_HUMAN | Cellular tumor antigen p53 | |
| 101 | Q53GA5 | Q53GA5_HUMAN | Cellular tumor antigen p53 (Fragment) | |
| 102 | A2A3F7 | A2A3F7_HUMAN | Transient receptor potential cation channel subfamily M | TRPM3 |
| | | | member 3 | |
| 103 | B4DMH2 | B4DMH2_HUMAN | Cellular tumor antigen p53 | |
| 104 | B7Z8X6 | B7Z8X6_HUMAN | Cellular tumor antigen p53 | |
| 105 | A0A141PNN3 | A0A141PNN3_HUMAN | Cellular tumor antigen p53 | TP63 |
| 106 | A0A141PNN4 | A0A141PNN4_HUMAN | Cellular tumor antigen p53 | TP63 |
| 107 | A0A087X1Q1 | A0A087X1Q1_HUMAN | Cellular tumor antigen p53 | TP53 |
| 108 | E5RMA8 | E5RMA8_HUMAN | Cellular tumor antigen p53 | TP53 |
| 109 | A0A0S2Z4N6 | A0A0S2Z4N6_HUMAN | Tumor protein p63 isoform 2 (Fragment) | TP63 |
| 110 | E9PBI7 | E9PBI7_HUMAN | Transient receptor potential cation channel subfamily M | TRPM3 |
| | | | member 3 | |
| 111 | A0A0G2JMR4 | A0A0G2JMR4_HUMAN | Transient receptor potential cation channel subfamily M | TRPM1 |
| | | | member 1 | |
| 112 | Q9H637 | Q9H637_HUMAN | cDNA: FLJ22628 fis, clone HSI06177 | |
| 113 | A0A0G2JPN6 | A0A0G2JPN6_HUMAN | Transient receptor potential cation channel subfamily M | TRPM1 |
| | | | member 1 | |
| 114 | A0A024R212 | A0A024R212_ HUMAN | Transient receptor potential cation channel, subfamily M, | TRPM1 hCG_37570 |
| | | | member 1, isoform CRA_b | |
| 115 | A0A0G2JMJ5 | A0A0G2JMJ5_HUMAN | Transient receptor potential cation channel subfamily M | TRPM1 |
| | | | member 1 | |
| 116 | H0YM61 | H0YM61_HUMAN | Transient receptor potential cation channel subfamily M | TRPM1 |
| | | | member 1 (Fragment) | |
| 117 | H0YKU7 | HOYKU7_HUMAN | Transient receptor potential cation channel subfamily M | TRPM1 |
| | | | member 1 (Fragment) | |
| 118 | A0A0A0MTQ9 | A0A0A0MTQ9_HUMAN | Transient receptor potential cation channel subfamily M | TRPM1 |
| | | | member 1 (Fragment) | |
| 119 | A2A3F3 | A2A3F3_HUMAN | Transient receptor potential cation channel subfamily M | TRPM3 |
| | | | member 3 | |

The amino acid and nucleotide sequences of each of these proteins and the TD thereof is incorportated herein by reference for use in the present invention and for potential inclusion in one or more claims herein.

TABLE 3
DNA sequences encoding Quad polypeptides

| SEQ ID NO: | POLYPEPTIDE | NUCLEOTIDE SEQUENCE |
|---|---|---|
| 13 | Quad 1 | ATGAGCCTCGGGCTCCTGTGCTGTGGGGCCTTTTCTCTCCTGTGGGCAGGTCCA |
| | | GTCAATGCCGGTGTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAAGACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAAGGC |
| | | TCTAGGCTGACCGTACTGGAGGACCTGAAAAACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGGCTTCTACCCCGACCATGTGGAGCTGAGCTGGTGGGTGAAT |
| | | GGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | CTAACAGACAGAGAATGGGCAGAAGAGTGGAAACATCTTGACCATCTGTTAAAC |
| | | TGCATAATGGACATGGTAGAAAAAACAAGGCGATCTCTCACCGTACTAAGGCGG |
| | | TGTCAAGAAGCAGACCCGGAAGAATTGAATTACTGGATCCGGCGGTACAGTGAC |
| | | GCCGAG |
| 14 | Quad 2 | ATGAGCCTCGGGCTCCTGTGCTGTGGGGCCTTTTCTCTCCTGTGGGCAGGTCCA |
| | | CTGAATCCCGGTCTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAACACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAACGC |
| | | TCTAGGCTGACCGTACTGGAGGACCTGAAAAACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGGCTTCTACCCCGACCATGTGGAGCTGAGCTGGTGGGTGAAT |
| | | GGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | GGAGGAGGTGGGAGCCTAACAGACAGAGAATGGGCAGAAGAGTGGAAACATCTT |
| | | GACCATCTGTTAAACTGCATAATGGACATGGTAGAAAAAACAAGGCGATCTCTC |
| | | ACCGTACTAAGGCGGTGTCAAGAAGCAGACCGGGAAGAATTGAATTACTGGATC |
| | | CGGCGGTACAGTGACGCCGAG |
| 15 | Quad 3 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAAGACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAAGGC |
| | | TCTAGGCTGACCGTACTGGAGGACCTGAAAPACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGCCTTCTACCCCGACCATGTGGAGCTGAGCTGGTGGGTGAAT |
| | | GGGAAGGAGGTGCACAGTGGGGTCTGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | CTAACAGACAGAGAATGGGCAGAAGAGTGGAAACATCTTGACCATCTGTTAAAC |
| | | TGCATAATGGACATGGTAGAAAAAACAAGGCGATCTCTCACCGTACTAAGGCGG |
| | | TGTCAAGAAGCAGACCGGGAAGAATTGAATTACTGGATCCGGCGGTACAGTGAC |
| | | GCCGAG |
| 16 | Quad 4 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAAGACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAAGGC |
| | | TCTAGGCTGACCGTACTGGAGGACCTGAAAAACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGGCTTCTACCCCGACCATGTGGAGCTGAGCTGGTGGGTGAAT |
| | | GGGAAGGAGGTGCACAGTGGGGTCTGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTCGACAAGAAGGTG |
| | | GGAGGAGGTGGGAGCCTAACAGACAGAGAATGGGCAGAAGAGTGGAAACATCTT |
| | | GACCATCTGTTAAACTGCATAATGGACATGGTAGAAAAAACAAGGCGATCTCTC |
| | | ACCGTACTAAGGCGGTGTCAAGAAGCAGACCGGGAAGAATTGAATTACTGGATC |
| | | CGGCGGTACAGTGACGCCGAG |
| 17 | Quad 5 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAAGACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAAGGC |
| | | TCTAGGCTGACCGTACTGGAGGACCTGAAAAACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGGCTTCTACCCCGACCATGTGGAGCTGAGCTGGTGGGTGAAT |
| | | GGGAAGGAGGTGCACAGTGGGGTCTGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACCTAACAGACAGAGAATGGGCAGAA |
| | | GAGTGGAAACATCTTGACCATCTGTTAAACTGCATAATGGACATGGTAGAAAAA |
| | | ACAAGGCGATCTCTCACCGTACTAAGGCGGTGTCAAGAAGCAGACCGGGAAGAA |
| | | TTGAATTACTGGATCCGGCGGTACAGTGACGCCGAG |
| 18 | Quad 6 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAAGACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAAGGC |
| | | TCTAGGCTGACCGTACTGGAGGACCTGAAAAACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGGCTTCTACCCCGACCATGTGGAGCTGAGCTGGTGGGTGAAT |
| | | GGGAAGGAGGTGCACAGTGGGGTCTGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACGGAGGAGGTGGGAGCCTAACAGAC |
| | | AGAGAATGGGCAGAAGAGTGGAAACATCTTGACCATCTGTTAAACTGCATAATG |
| | | GACATGGTAGAAAAAACAAGGCGATCTCTCACCGTACTAAGGCGGTGTCAAGAA |
| | | GCAGACCGGGAAGAATTGAATTACTGGATCCGGCGGTACAGTGACGCCGAG |
| 19 | Quad 7 | ATGGAGACCCTCTTGGGCCTGCTTATCCTTTGGCTGCAGCTGCAATGGGTGAGC |
| | | AGCAAACAGGAGGTGACACAGATTCCTGCAGCTCTGAGTGTCCCAGAAGGAGAA |
| | | AACTTGGTTCTCAACTGCAGTTTCACTGATAGCGCTATTTACAACCTCCAGTGG |
| | | TTTAGGCAGGACCCTGGGAAAGGTCTCACATCTCTGTTGCTTATTACACCTTGG |
| | | CAGAGAGAGCAAACAAGTGGAAGACTTAATGCCTCGCTGGATAAATCATCAGGA |
| | | CGTAGTACTTTATACATTGCAGCTTCTCAGCCTGGTGACTCAGCCACCTACCTC |
| | | TGTGCTGTGAGGCCCCTGCTTGACGGAACATACATACCTACATTTGGAAGAGGA |
| | | ACCAGCCTTATTGTTCATCCGTATATCCAGAACCCTGACCCTGCCGTGTACCAG |
| | | CTGAGAGACTCTAAATCCAGTGACAAGTCTGTCTGCCTATTCACCGATTTTGAT |
| | | TCTCAAACAAATGTGTCACAAAGTAAGGATTCTGATGTGTATATCACAGACAAA |
| | | ACTGTGCTAGACATGAGGTCTATGGACTTCAAGAGCAACAGTGCTGTGGCCTGG |
| | | AGCAACAAATCTGACTTTGCATGTGCAAACGCCTTCAACAACAGCATTATTCCA |
| | | GAAGACACCTTCTTCCCCAGCCCAGAAAGTTCCACGGTGGCCGCTCCCTCCGTG |
| | | TTCATCTTCCCACCTTCCGACGAGCAGCTGAAGTCCGGCACCGCTTCTGTCGTG |
| | | TGCCTGCTGAACAACTTCTACCCCCGCGAGGCCAAGGTGCAGTGGAAGGTGGAC |
| | | AACGCCCTGCAGTCCGGCAACTCCCAGGAATCCGTGACCGAGCAGGACTCCAAG |
| | | GACAGCACCTACTCCCTGTCCTCCACCCTGACCCTGTCCAAGGCCGACTACGAG |
| | | AAGCACAAGGTGTACGCCTGCGAAGTGACCCACCAGGGCCTGTCTAGCCCCGTG |
| | | ACCAAGTCTTTCAACCGGGGCGAGTGT |
| 20 | Quad 8 | ATGGAGACCCTCTTGGGCCTGCTTATCCTTTGGCTGCAGCTGCAATGGGTGAGC |
| | | AGCAAACAGGAGGTGACACAGATTCCTGCAGCTCTGAGTGTCCCAGAAGGAGAA |
| | | AACTTGGTTCTCAACTGCAGTTTCACTGATAGCGCTATTTACAACCTCCAGTGG |
| | | TTTAGGCAGGACCCTGGGAAAGGTCTCACATCTCTGTTGCTTATTACACCTTGG |
| | | CAGAGAGAGCAAACAAGTGGAAGACTTAATGCCTCGCTGGATAAATCATCAGGA |
| | | CGTAGTACTTTATACATTGCAGCTTCTCAGCCTGGTGACTCAGCCACCTACCTC |
| | | TGTGCTGTGAGGCCCCTGCTTGACGGAACATACATACCTACATTTGGAAGAGGA |
| | | ACCAGCCTTATTGTTCATCCGTATATCCAGAACCCTGACCCTGCCGTGTACCAG |
| | | CTGAGAGACTCTAAATCCAGTGACAAGTCTGTCTGCCTATTCACCGATTTTGAT |
| | | TCTCAAACAAATGTGTCACAAAGTAAGGATTCTGATGTGTATATCACAGACAAA |
| | | TGTGTGCTAGACATGAGGTCTATGGACTTCAAGAGCAACAGTGCTGTGGCCTGG |
| | | AGCAACAAATCTGACTTTGCATGTGCAAACGCCTTCAACAACAGCATTATTCCA |
| | | GAAGACACCTTCTTCCCCAGCCCAGAAAGTTCCACGGTGGCCGCTCCCTCCGTG |
| | | TTCATCTTCCCACCTTCCGACGAGCAGCTGAAGTCCGGCACCGCTTCTGTCGTG |
| | | TGCCTGCTGAACAACTTCTACCCCCGCGAGGCCAAGGTGCAGTGGAAGGTGGAC |
| | | AACGCCCTGCAGTCCGGCAACTCCCAGGAATCCGTGACCGAGCAGGACTCCAAG |
| | | GACAGCACCTACTCCCTGTCCTCCACCCTGACCCTGTCCAAGGCCGACTACGAG |
| | | AAGCACAAGGTGTACGCCTGCGAAGTGACCCACCAGGGCCTGTCTAGCCCCGTG |
| | | ACCAAGTCTTTCAACCGGGGCGAGTGT |
| 21 | Quad 9 | ATGGAGACCCTCTTGGGCCTGCTTATCCTTTGGCTGCAGCTGCAATGGGTGAGC |
| | | AGCAAACAGGAGGTGACACAGATTCCTGCAGCTCTGAGTGTCCCAGAAGGAGAA |
| | | AACTTGGTTCTCAACTGCAGTTTCACTGATAGCGCTATTTACAACCTCCAGTGG |
| | | TTTAGGCAGGACCCTGGGAAAGGTCTCACATCTCTGTTGCTTATTACACCTTGG |
| | | CAGAGAGAGCAAACAAGTGGAAGACTTAATGCCTCGCTGGATAAATCATCAGGA |
| | | CGTAGTACTTTATACATTGCAGCTTCTCAGCCTGGTGACTCAGCCACCTACCTC |
| | | TGTGCTGTGAGGCCCCTGCTTGACGGAACATACATACCTACATTTGGAAGAGGA |
| | | ACCAGCCTTATTGTTCATCCGTATATCCAGAACCCTGACCCTGCCGTGTACCAG |
| | | CTGAGAGACTCTAAATCCAGTGACAAGTCTGTCTGCCTATTCACCGATTTTGAT |
| | | TCTCAAACAAATGTGTCACAAAGTAAGGATTCTGATGTGTATATCACAGACAAA |
| | | TGTGTGCTAGACATGAGGTCTATGGACTTCAAGAGCAACAGTGCTGTGGCCTGG |
| | | AGCAACAAATCTGACTTTGCATGTGCAAACGCCTTCAACAACAGCATTATTCCA |
| | | GAAGACACCTTCTTCCCCAGCCCAGAAAGTTCC |
| 22 | Quad 10 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAAGACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAAGGC |
| | | TCTAGGCTGACCGTACTGGAGGACCTGAAAAACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGGCTTCTACCCCGACCATGTGGAGCTGAGCTGGTGGGTGAAT |
| | | GGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | CTAACAGACAGAGAATGGGCAGAAGAGTGGAAACATCTTGACCATCTGTTAAAC |
| | | TGCATAATGGACATGGTAGAAAAAACAAGGCGATCTCTCACCGTACTAAGGCGG |
| | | TGTCAAGAAGCAGACCGGGAAGAATTGAATTACTGGATCCGGCGGTACAGTGAC |
| | | GCCGAGGCACCTACTTCAAGTTCTACAAAGAAAACACAGCTACAACTGGAGCAT |
| | | TTACTGCTGGATTTACAGATGATTTTGAATGGAATTAATAATTACAAGAATCCC |
| | | AAACTCACCAGGATGCTCACATTTAAGTTTTACATGCCCAAGAAGGCCACAGAA |
| | | CTGAAACATCTTCAGTGTCTAGAAGAAGAACTCAAACCTCTGGAGGAAGTGCTA |
| | | AATTTAGCTCAAAGCAAAAACTTTCACTTAAGACCCAGGGACTTAATCAGCAAT |
| | | ATCAACGTAATAGTTCTGGAACTAAAGGGATCTGAAACAACATTCATGTGTGAA |
| | | TATGCTGATGAGACAGCAACCATTGTAGAATTTCTGAACAGATGGATTACCTTT |
| | | TGTCAAAGCATCATCTCAACACTGACT |
| 23 | Quad 11 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAAGACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAAGGC |
| | | TCTAGGCTGACCGTACTGGAGGACCTGAAAAACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGGCTTCTACCCCGACCATGTGGAGCTGAGCTGGTGGGTGAAT |
| | | GGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | GGAGGAGGTGGGAGCCTAACAGACAGAGAATGGGCAGAAGAGTGGAAACATCTT |
| | | GACCATCTGTTAAACTGCATAATGGACATGGTAGAAAAAACAAGGCGATCTCTC |
| | | ACCGTACTAAGGCGGTGTCAAGAAGCAGACCGGGAAGAATTGAATTACTGGATC |
| | | CGGCGGTACAGTGACGCCGAGGCACCTACTTCAAGTTCTACAAAGAAAACACAG |
| | | CTACAACTGGAGCATTTACTGCTGGATTTACAGATGATTTTGAATGGAATTAAT |
| | | AATTACAAGAATCCCAAACTCACCAGGATGCTCACATTTAAGTTTTACATGCCC |
| | | AAGAAGGCCACAGAACTGAAACATCTTCAGTGTCTAGAAGAAGAACTCAAACCT |
| | | CTGGAGGAAGTGCTAAATTTAGCTCAAAGCAAAAACTTTCACTTAAGACCCAGG |
| | | GACTTAATCAGCAATATCAACGTAATAGTTCTGGAACTAAAGGGATCTGAAACA |
| | | ACATTCATGTGTGAATATGCTGATGAGACAGCAACCATTGTAGAATTTCTGAAC |
| | | AGATGGATTACCTTTTGTCAAAGCATCATCTCAACACTGACT |
| 24 | Quad 12 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAAGACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAAGGC |
| | | TCTAGGCTGACCGTACTGGAGGACCTGAAAAACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGGCTTCTACCCCGACCATGTGGAGCTGAGCTGGTGGGTGAAT |
| | | GGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | GAACCCAAGTCCTGCGACAAGACCCACACCTGTCCCCCTTGTCCTCTAACAGAC |
| | | AGAGAATGGGCAGAAGAGTGGAAACATCTTGACCATCTGTTAAACTGCATAATG |
| | | GACATGGTAGAAAAAACAAGGCGATCTCTCACCGTACTAAGGCGGTGTCAAGAA |
| | | GCAGACCGGGAAGAATTGAATTACTGGATCCGGCGGTACAGTGACGCCGAG |
| 25 | Quad 13 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAAGACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAAGGC |
| | | TCTAGGCTGACCGTACTGGAGGACCTGAAAAACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGGCTTCTACCCCGACCATGTGGAGCTGAGCTGGTGGGTGAAT |
| | | GGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | GAACCCAAGTCCTGCGACAAGACCCACACCTGTCCCCCTTGTCCTGGAGGAGGT |
| | | GGGAGCCTAACAGACAGAGAATGGGCAGAAGAGTGGAAACATCTTGACCATCTG |
| | | TTAAACTGCATAATGGACATGGTAGAAAAAACAAGGCGATCTCTCACCGTACTA |
| | | AGGCGGTGTCAAGAAGCAGACCGGGAAGAATTGAATTACTGGATCCGGCGGTAC |
| | | AGTGACGCCGAG |
| 26 | Quad 14 | ATGGAGCTGGGGCTGAGCTGGGTGGTCCTGGCTGCTCTACTACAAGGTGTCCAG |
| | | GCTCAGGTTCAGCTGGTTGAAAGCGGTGGTGCACTGGTTCAGCCTGGTGGTAGC |
| | | CTGCGTCTGAGCTGTGCAGCAAGCGGTTTTCCGGTTAATCGTTATAGCATGCGT |
| | | TGGTATCGTCAGGCACCGGGTAAAGAACGTGAATGGGTTGCAGGTATGAGCAGT |
| | | GCCGGTGATCGTAGCAGCTATGAAGATAGCGTTAAAGGTCGTTTTACCATCAGC |
| | | CGTGATGATGCACGTAATACCGTTTATCTGCAAATGAATAGCCTGAAACCGGAA |
| | | GATACCGCAGTGTATTATTGCAATGTTAACGTGGGCTTTGAATATTGGGGTCAG |
| | | GGCACCCAGGTTACCGTTAGCAGCAAACTAACAGACAGAGAATGGGCAGAAGAG |
| | | TGGAAACATCTTGACCATCTGTTAAACTGCATAATGGACATGGTAGAAAAAACA |
| | | AGGCGATCTCTCACCGTACTAAGGCGGTGTCAAGAAGCAGACCGGGAAGAATTG |
| | | AATTACTGGATCCGGCGGTACAGTGACGCCGAG |
| 27 | Quad 15 | ATGGAGCTGGGGCTGAGCTGGGTGGTCCTGGCTGCTCTACTACAAGGTGTCCAG |
| | | GCTCAGGTTCAGCTGGTTGAAAGCGGTGGTGCACTGGTTCAGCCTGGTGGTAGC |
| | | CTGCGTCTGAGCTGTGCAGCAAGCGGTTTTCCGGTTAATCGTTATAGCATGCGT |
| | | TGGTATCGTCAGGCACCGGGTAAAGAACGTGAATGGGTTGCAGGTATGAGCAGT |
| | | GCCGGTGATCGTAGCAGCTATGAAGATAGCGTTAAAGGTCGTTTTACCATCAGC |
| | | CGTGATGATGCACGTAATACCGTTTATCTGCAAATGAATAGCCTGAAACCGGAA |
| | | GATACCGCAGTGTATTATTGCAATGTTAACGTGGGCTTTGAATATTGGGGTCAG |
| | | GGCACCCAGGTTACCGTTAGCAGCAAAGGAGGAGGTGGGAGCCTAACAGACAGA |
| | | GAATGGGCAGAAGAGTGGAAACATCTTGACCATCTGTTAAACTGCATAATGGAC |
| | | ATGGTAGAAAAAACAAGGCGATCTCTCACCGTACTAAGGCGGTGTCAAGAAGCA |
| | | GACCGGGAAGAATTGAATTACTGGATCCGGCGGTACAGTGACGCCGAG |
| 28 | Quad 18 | ATGGAGCTGGGGCTGAGCTGGGTGGTCCTGGCTGCTCTACTACAAGGTGTCCAG |
| | | GCTCAGGTTCAGCTGGTTGAAAGCGGTGGTGCACTGGTTCAGCCTGGTGGTAGC |
| | | CTGCGTCTGAGCTGTGCAGCAAGCGGTTTTCCGGTTAATCGTTATAGCATGCGT |
| | | TGGTATCGTCAGGCACCGGGTAAAGAACGTGAATGGGTTGCAGGTATGAGCAGT |
| | | GCCGGTGATCGTAGCAGCTATGAAGATAGCGTTAAAGGTCGTTTTACCATCAGC |
| | | CGTGATGATGCACGTAATACCGTTTATCTGCAAATGAATAGCCTGAAACCGGAA |
| | | GATACCGCAGTGTATTATTGCAATGTTAACGTGGGCTTTGAATATTGGGGTCAG |
| | | GGCACCCAGGTTACCGTTAGCAGCAAACTAACAGACAGAGAATGGGCAGAAGAG |
| | | TGGAAACATCTTGACCATCTGTTAAACTGCATAATGGACATGGTAGAAAAAACA |
| | | AGGCGATCTCTCACCGTACTAAGGCGGTGTCAAGAAGCAGACCGGGAAGAATTG |
| | | AATTACTGGATCCGGCGGTACAGTGACGCCGAGGCACCTACTTCAAGTTCTACA |
| | | AAGAAAACACAGCTACAACTGGAGCATTTACTGCTGGATTTACAGATGATTTTG |
| | | AATGGAATTAATAATTACAAGAATCCCAAACTCACCAGGATGCTCACATTTAAG |
| | | TTTTACATGCCCAAGAAGGCCACAGAACTGAAACATCTTCAGTGTCTAGAAGAA |
| | | GAACTCAAACCTCTGGAGGAAGTGCTAAATTTAGCTCAAAGCAAAAACTTTCAC |
| | | TTAAGACCCAGGGACTTAATCAGCAATATCAACGTAATAGTTCTGGAACTAAAG |
| | | GGATCTGAAACAACATTCATGTGTGAATATGCTGATGAGACAGCAACCATTGTA |
| | | GAATTTCTGAACAGATGGATTACCTTTTGTCAAAGCATCATCTCAACACTGACT |
| 29 | Quad 19 | ATGGAGCTGGGGCTGAGCTGGGTGGTCCTGGCTGCTCTACTACAAGGTGTCCAG |
| | | GCTCAGGTTCAGCTGGTTGAAAGCGGTGGTGCACTGGTTCAGCCTGGTGGTAGC |
| | | CTGCGTCTGAGCTGTGCAGCAAGCGGTTTTCCGGTTAATCGTTATAGCATGCGT |
| | | TGGTATCGTCAGGCACCGGGTAAAGAACGTGAATGGGTTGCAGGTATGAGCAGT |
| | | GCCGGTGATCGTAGCAGCTATGAAGATAGCGTTAAAGGTCGTTTTACCATCAGC |
| | | CGTGATGATGCACGTAATACCGTTTATCTGCAAATGAATAGCCTGAAACCGGAA |
| | | GATACCGCAGTGTATTATTGCAATGTTAACGTGGGCTTTGAATATTGGGGTCAG |
| | | GGCACCCAGGTTACCGTTAGCAGCAAAGGAGGAGGTGGGAGCCTAACAGACAGA |
| | | GAATGGGCAGAAGAGTGGAAACATCTTGACCATCTGTTAAACTGCATAATGGAC |
| | | ATGGTAGAAAAAACAAGGCGATCTCTCACCGTACTAAGGCGGTGTCAAGAAGCA |
| | | GACCGGGAAGAATTGAATTACTGGATCCGGCGGTACAGTGACGCCGAGGCACCT |
| | | ACTTCAAGTTCTACAAAGAAAACACAGCTACAACTGGAGCATTTACTGCTGGAT |
| | | TTACAGATGATTTTGAATGGAATTAATAATTACAAGAATCCCAAACTCACCAGG |
| | | ATGCTCACATTTAAGTTTTACATGCCCAAGAAGGCCACAGAACTGAAACATCTT |
| | | CAGTGTCTAGAAGAAGAACTCAAACCTCTGGAGGAAGTGCTAAATTTAGCTCAA |
| | | AGCAAAAACTTTCACTTAAGACCCAGGGACTTAATCAGCAATATCAACGTAATA |
| | | GTTCTGGAACTAAAGGGATCTGAAACAACATTCATGTGTGAATATGCTGATGAG |
| | | ACAGCAACCATTGTAGAATTTCTGAACAGATGGATTACCTTTTGTCAAAGCATC |
| | | ATCTCAACACTGACT |
| 30 | Quad 20 | ATGGAGACCCTCTTGGGCCTGCTTATCCTTTGGCTGCAGCTGCAATGGGTGAGC |
| | | AGCAAACAGGAGGTGACACAGATTCCTGCAGCTCTGAGTGTCCCAGAAGGAGAA |
| | | AACTTGGTTCTCAACTGCAGTTTCACTGATAGCGCTATTTACAACCTCCAGTGG |
| | | TTTAGGCAGGACCCTGGGAAAGGTCTCACATCTCTGTTGCTTATTACACCTTGG |
| | | CAGAGAGAGCAAACAAGTGGAAGACTTAATGCCTCGCTGGATAAATCATCAGGA |
| | | CGTAGTACTTTATACATTGCAGCTTCTCAGCCTGGTGACTCAGCCACCTACCTC |
| | | TGTGCTGTGAGGCCCCTGCTTGACGGAACATACATACCTACATTTGGAAGAGGA |
| | | ACCAGCCTTATTGTTCATCCGTATATCCAGAACCCTGACCCTGCCGTGTACCAG |
| | | CTGAGAGACTCTAAATCCAGTGACAAGTCTGTCTGCCTATTCACCGATTTTGAT |
| | | TCTCAAACAAATGTGTCACAAAGTAAGGATTCTGATGTGTATATCACAGACAAA |
| | | ACTGTGCTAGACATGAGGTCTATGGACTTCAAGAGCAACAGTGCTGTGGCCTGG |
| | | AGCAACAAATCTGACTTTGCATGTGCAAACGCCTTCAACAACAGCATTATTCCA |
| | | GAAGACACCTTCTTCCCCAGCCCAGAAAGTTCCGCCAGCACCAAGGGCCCCTCT |
| | | GTGTTCCCTCTGGCCCCTTCCAGCAAGTCCACCTCTGGCGGAACAGCCGCTCTG |
| | | GGCTGCCTCGTGAAGGACTACTTCCCCGAGCCTGTGACCGTGTCCTGGAACTCT |
| | | GGCGCTCTGACCAGCGGAGTGCACACCTTCCCTGCTGTGCTGCAGTCCTCCGGC |
| | | CTGTACTCCCTGTCCTCCGTCGTGACCGTGCCTTCCAGCTCTCTGGGCACCCAG |
| | | ACCTACATCTGCAACGTGAACCACAAGCCCTCCAACACCAAGGTGGACAAGAAG |
| | | GTGTTGCATGGCACACGTCAAGAAGAAATGATTGATCACAGACTAACAGACAGA |
| | | GAATGGGCAGAAGAGTGGAAACATCTTGACCATCTGTTAAACTGCATAATGGAC |
| | | ATGGTAGAAAAAACAAGGCGATCTCTCACCGTACTAAGGCGGTGTCAAGAAGCA |
| | | GACCGGGAAGAATTGAATTACTGGATCCGGCGGTACAGTGACGCCGAG |
| 31 | Quad 21 | ATGGAGACCCTCTTGGGCCTGCTTATCCTTTGGCTGCAGCTGCAATGGGTGAGC |
| | | AGCAAACAGGAGGTGACACAGATTCCTGCAGCTCTGAGTGTCCCAGAAGGAGAA |
| | | AACTTGGTTCTCAACTGCAGTTTCACTGATAGCGCTATTTACAACCTCCAGTGG |
| | | TTTAGGCAGGACCCTGGGAAAGGTCTCACATCTCTGTTGCTTATTACACCTTGG |
| | | CAGAGAGAGCAAACAAGTGGAAGACTTAATGCCTCGCTGGATAAATCATCAGGA |
| | | CGTAGTACTTTATACATTGCAGCTTCTCAGCCTGGTGACTCAGCCACCTACCTC |
| | | TGTGCTGTGAGGCCCCTGCTTGACGGAACATACATACCTACATTTGGAAGAGGA |
| | | ACCAGCCTTATTGTTCATCCGTATATCCAGAACCCTGACCCTGCCGTGTACCAG |
| | | CTGAGAGACTCTAAATCCAGTGACAAGTCTGTCTGCCTATTCACCGATTTTGAT |
| | | TCTCAAACAAATGTGTCACAAAGTAAGGATTCTGATGTGTATATCACAGACAAA |
| | | ACTGTGCTAGACATGAGGTCTATGGACTTCAAGAGCAACAGTGCTGTGGCCTGG |
| | | AGCAACAAATCTGACTTTGCATGTGCAAACGCCTTCAACAACAGCATTATTCCA |
| | | GAAGACACCTTCTTCCCCAGCCCAGAAAGTTCCGCCAGCACCAAGGGCCCCTCT |
| | | GTGTTCCCTCTGGCCCCTTCCAGCAAGTCCACCTCTGGCGGAACAGCCGCTCTG |
| | | GGCTGCCTCGTGAAGGACTACTTCCCCGAGCCTGTGACCGTGTCCTGGAACTCT |
| | | GGCGCTCTGACCAGCGGAGTGCACACCTTCCCTGCTGTGCTGCAGTCCTCCGGC |
| | | CTGTACTCCCTGTCCTCCGTCGTGACCGTGCCTTCCAGCTCTCTGGGCACCCAG |
| | | ACCTACATCTGCAACGTGAACCACAAGCCCTCCAACACCAAGGTGGACAAGAAG |
| | | GTGGGAGGAGGTGGGAGCTTGCATGGCACACGTCAAGAAGAAATGATTGATCAC |
| | | AGACTAACAGACAGAGAATGGGCAGAAGAGTGGAAACATCTTGACCATCTGTTA |
| | | AACTGCATAATGGACATGGTAGAAAAAACAAGGCGATCTCTCACCGTACTAAGG |
| | | CGGTGTCAAGAAGCAGACCGGGAAGAATTGAATTACTGGATCCGGCGGTACAGT |
| | | GACGCCGAGGACTTAAAA |
| 32 | Quad 22 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAG |
| | | AGCATGACACTGCAGTGTGCCCAGGATATGAACCATGAATACATGTCCTGGTAT |
| | | CGACAAGACCCAGGCATGGGGCTGAGGCTGATTCATTACTCAGTTGCCATCCAG |
| | | ACAACTGACCAAGGAGAAGTCCCCAATGGCTACAATGTCTCCAGATCAACCATC |
| | | GAGGATTTCCCGCTCAGGCTGCTGTCGGCTGCTCCCTCCCAGACATCTGTGTAC |
| | | TTCTGTGCCAGCAGTTACCTGGGGAACACCGGGGAGCTGTTTTTTGGAGAAGGC |
| | | TCTAGGCTGACCGTACTGGAGGACCTGAAAAACGTGTTCCCACCCGAGGTCGCT |
| | | GTGTTTGAGCCATCAGAAGCAGAGATCTCCCACACCCAAAAGGCCACACTGGTG |
| | | TGCCTGGCCACAGGCTTCTACCCCGACCATGTGGAGCTGAGCTGGTGGGTGAAT |
| | | GGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCC |
| | | ACCTTCTGGCAGGACCCCCGCAACCACTTCCGCTGTCAAGTCCAGTTCTACGGG |
| | | CTCTCGGAGAATGACGAGTGGACCCAGGATAGGGCCAAACCCGTCACCCAGATC |
| | | GTCAGCGCCGAGGCCTGGGGTAGAGCAGACACGGTGGCCGCTCCCTCCGTGTTC |
| | | ATCTTCCCACCTTCCGACGAGCAGCTGAAGTCCGGCACCGCTTCTGTCGTGTGC |
| | | CTGCTGAACAACTTCTACCCCCGCGAGGCCAAGGTGCAGTGGAAGGTGGACAAC |
| | | GCCCTGCAGTCCGGCAACTCCCAGGAATCCGTGACCGAGCAGGACTCCAAGGAC |
| | | AGCACCTACTCCCTGTCCTCCACCCTGACCCTGTCCAAGGCCGACTACGAGAAG |
| | | CACAAGGTGTACGCCTGCGAAGTGACCCACCAGGGCCTGTCTAGCCCCGTGACC |
| | | AAGTCTTTCAACCGGGGCGAGTGT |
| 33 | Quad 23 | ATGAACTTCGGGCTCAGCTTGATTTTCCTTGCCCTTATTTTAAAAGGTGTCCAG |
| | | TGTGAGGTGCAACTGGTGGAGTCTGGGGGAGGCTTAGTGAAGCCTGGGGGGTCC |
| | | CTGAAACTCTCCTGTGCAGCCTCTGGACTCACTTTCAGTAGCTATGCCATGTCT |
| | | TGGGTTCGCCAGACTCCAGAGAAGAGGCTGGAGTGGGTCGCATCCATTAGTAGT |
| | | GGTGGTTTCACCTACTATCCAGACAGTGTGAAGGGCCGATTCACCATCTCCAGA |
| | | GATAATGCCAGGAACATCCTGTATCTGCAAATGAGCAGTCTGAGGTCTGAGGAC |
| | | ACGGCCATGTATTACTGTGCAAGAGACGAGGTACGGGGGTACCTCGATGTCTGG |
| | | GGCGCAGGGACCACGGTCACCGTTTCCTCGGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | CTAACAGACAGAGAATGGGCAGAAGAGTGGAAACATCTTGACCATCTGTTAAAC |
| | | TGCATAATGGACATGGTAGAAAAAACAAGGCGATCTCTCACCGTACTAAGGCGG |
| | | TGTCAAGAAGCAGACCGGGAAGAATTGAATTACTGGATCCGGCGGTACAGTGAC |
| | | GCCGAG |
| 34 | Quad 24 | ATGAACTTCGGGCTCAGCTTGATTTTCCTTGCCCTTATTTTAAAAGGTGTCCAG |
| | | TGTGAGGTGCAACTGGTGGAGTCTGGGGGAGGCTTAGTGAAGCCTGGGGGGTCC |
| | | CTGAAACTCTCCTGTGCAGCCTCTGGACTCACTTTCAGTAGCTATGCCATGTCT |
| | | TGGGTTCGCCAGACTCCAGAGAAGAGGCTGGAGTGGGTCGCATCCATTAGTAGT |
| | | GGTGGTTTCACCTACTATCCAGACAGTGTGAAGGGCCGATTCACCATCTCCAGA |
| | | GATAATGCCAGGAACATCCTGTATCTGCAAATGAGCAGTCTGAGGTCTGAGGAC |
| | | ACGGCCATGTATTACTGTGCAAGAGACGAGGTACGGGGGTACCTCGATGTCTGG |
| | | GGCGCAGGGACCACGGTCACCGTTTCCTCGGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | GGAGGAGGTGGGAGCCTAACAGACAGAGAATGGGCAGAAGAGTGGAAACATCTT |
| | | GACCATCTGTTAAACTGCATAATGGACATCGTAGAAAAAACAACGCGATCTCTC |
| | | ACCGTACTAAGGCGGTGTCAAGAAGCAGACCGGGAAGAATTGAATTACTGGATC |
| | | CGGCGGTACAGTGACGCCGAG |
| 35 | Quad 25 | ATGAAATACCTATTGCCTACGGCAGCCGCTGGATTGTTATTACTCGCGGCCCAG |
| | | CCGGCCATGGCGGCCTACAAAGATATCCAGATGACACAGACTACATCCTCCCTG |
| | | TCTGCCTCTCTGGGAGACAGAGTCACCATCAGTTGCAGTGCAAGTCAGGGCATT |
| | | AGCAATTATTTAAACTGGTATCAGCAGAAACCAGATGGAACTGTTAAACTCCTG |
| | | ATCTATTACACATCAAGTTTACACTCAGGAGTCCCATCAAGGTTCAGTGGCAGT |
| | | GGGTCTGGGACAGATTATTCTCTCACCATCAGCAACCTGGAACCTGAAGATATT |
| | | GCCACTTATTATTGTCAGCAGTATAGCAAGCTTCCGTACACGTTCGGAGGGGGG |
| | | ACCAAGCTGGAAATAAAACGTACGGTGGCCGCTCCCTCCGTGTTCATCTTCCCA |
| | | CCTTCCGACGAGCAGCTGAAGTCCGGCACCGCTTCTGTCGTGTGCCTGCTGAAC |
| | | AACTTCTACCCCCGCGAGGCCAAGGTGCAGTGGAAGGTGGACAACGCCCTGCAG |
| | | TCCGGCAACTCCCAGGAATCCGTGACCGAGCAGGACTCCAAGGACAGCACCTAC |
| | | TCCCTGTCCTCCACCCTGACCCTGTCCAAGGCCGACTACGAGAAGCACAAGGTG |
| | | TACGCCTGCGAAGTGACCCACCAGGGCCTGTCTAGCCCCGTGACCAAGTCTTTC |
| | | AACCGGGGCGAGTGT |
| 36 | Quad 26 | ATGAACTTCGGGCTCAGCTTGATTTTCCTTGCCCTTATTTTAAAAGGTGTCCAG |
| | | TGTGAGGTGCAACTGGTGGAGTCTGGGGGAGGCTTAGTGAAGCCTGGGGGGTCC |
| | | CTGAAACTCTCCTGTGCAGCCTCTGGACTCACTTTCAGTAGCTATGCCATGTCT |
| | | TGGGTTCGCCAGACTCCAGAGAAGAGGCTGGAGTGGGTCGCATCCATTAGTAGT |
| | | GGTGGTTTCACCTACTATCCAGACAGTGTGAAGGGCCGATTCACCATCTCCAGA |
| | | GATAATGCCAGGAACATCCTGTATCTGCAAATGAGCAGTCTGAGGTCTGAGGAC |
| | | ACGGCCATGTATTACTGTGCAAGAGACGAGGTACGGGGGTACCTCGATGTCTGG |
| | | GGCGCAGGGACCACGGTCACCGTTTCCTCGGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | GAACCCAAGTCCTGCGACAAGACCCACACCTGTCCCCCTTGTCCTCTAACAGAC |
| | | AGAGAATGGGCAGAAGAGTGGAAACATCTTGACCATCTGTTAAACTGCATAATG |
| | | GACATGGTAGAAAAAACAAGGCGATCTCTCACCGTACTAAGGCGGTGTCAAGAA |
| | | GCAGACCGGGAAGAATTGAATTACTGGATCCGGCGGTACAGTGACGCCGAG |
| 37 | Quad 27 | ATGAACTTCGGGCTCAGCTTGATTTTCCTTGCCCTTATTTTAAAAGGTGTCCAG |
| | | TGTGAGGTGCAACTGGTGGAGTCTGGGGGAGGCTTAGTGAAGCCTGGGGGGTCC |
| | | CTGAAACTCTCCTGTGCAGCCTCTGGACTCACTTTCAGTAGCTATGCCATGTCT |
| | | TGGGTTCGCCAGACTCCAGAGAAGAGGCTGGAGTGGGTCGCATCCATTAGTAGT |
| | | GGTGGTTTCACCTACTATCCAGACAGTGTGAAGGGCCGATTCACCATCTCCAGA |
| | | GATAATGCCAGGAACATCCTGTATCTGCAAATGAGCAGTCTGAGGTCTGAGGAC |
| | | ACGGCCATGTATTACTGTGCAAGAGACGAGGTACGGGGGTACCTCGATGTCTGG |
| | | GGCGCAGGGACCACGGTCACCGTTTCCTCGGCCAGCACCAAGGGCCCCTCTGTG |
| | | TTCCCTCTGGCCCCTTCCAGCAAGTCCACCTCTGGCGGAACAGCCGCTCTGGGC |
| | | TGCCTCGTGAAGGACTACTTCCCCGAGCCTGTGACCGTGTCCTGGAACTCTGGC |
| | | GCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACC |
| | | TACATCTGCAACGTGAACCACAAGCCCTCCAACACCAAGGTGGACAAGAAGGTG |
| | | GAACCCAAGTCCTGCGACAAGACCCACACCTGTCCCCCTTGTCCTGGAGGAGGT |
| | | GGGAGCCTAACAGACAGAGAATGGGCAGAAGAGTGGAAACATCTTGACCATCTG |
| | | TTAAACTGCATAATGGACATGGTAGAAAAAACAAGGCGATCTCTCACCGTACTA |
| | | AGGCGGTGTCAAGAAGCAGACCGGGAAGAATTGAATTACTGGATCCGGCGGTAC |
| | | AGTGACGCCGAG |
| 38 | Quad 28 | ATGGGATGGTOTTGTATAATTCTGTTCCTGGTGGCAACAGCAACAGGAGTGCAT |
| | | AGCGAGGTGCAGCTGGTGGAGTCTGGGGGAGGCTTGGTACAGCCCGGCAGGTCC |
| | | CTGAGACTCTCCTGTGCGGCCTCTGGATTCACCTTTGATGATTATGCCATGCAC |
| | | TGGGTCCGGCAAGCTCCAGGGAAGGGCCTGGAATGGGTCTCAGCTATCACTTGG |
| | | AATAGTGGTCACATAGACTATGCGGACTCTGTGGAGGGCCGATTCACCATCTCC |
| | | AGAGACAACGCCAAGAACTCCCTGTATCTGCAAATGAACAGTCTGAGAGCTGAG |
| | | GATACGGCCGTATATTACTGTGCGAAAGTCTCGTACCTTAGCACCGCGTCCTCC |
| | | CTTGACTATTGGGGCCAAGGTACCCTGGTCACCGTCTCGAGTGCCAGCACCAAG |
| | | GGCCCCTCTGTGTTCCCTCTGGCCCCTTCCAGCAAGTCCACCTCTGGCGGAACA |
| | | GCCGCTCTGGGCTGCCTCGTGAAGGACTACTTCCCCGAGCCTGTGACCGTGTCC |
| | | TGGAACTCTGGCGCTCTGACCAGCGGAGTGCACACCTTCCCTGCTGTGCTGCAG |
| | | TCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTG |
| | | GACAAGAAGGTGTTGCATGGCACACGTCAAGAAGAAATGATTGATCACAGACTA |
| | | ACAGACAGAGAATGGGCAGAAGAGTGGAAACATCTTGACCATCTGTTAAACTGC |
| | | ATAATGGACATGGTAGAAAAAACAAGGCGATCTCTCACCGTACTAAGGCGGTGT |
| | | CAAGAAGCAGACCGGGAAGAATTGAATTACTGGATCCGGCGGTACAGTGACGCC |
| | | GAG |
| 39 | Quad 29 | ATGGGATGGTCTTGTATAATTCTGTTCCTGGTGGCAACAGCAACAGGAGTGCAT |
| | | AGCGAGGTGCAGCTGGTGGAGTCTGGGGGAGGCTTGGTACAGCCCGGCAGGTCC |
| | | CTGAGACTCTCCTGTGCGGCCTCTGGATTCACCTTTGATGATTATGCCATGCAC |
| | | TGGGTCCGGCAAGCTCCAGGGAAGGGCCTGGAATCCCTCTCAGCTATCACTTGG |
| | | AATAGTGGTCACATAGACTATGCGGACTCTGTGGAGGGCCGATTCACCATCTCC |
| | | AGAGACAACGCCAAGAACTCCCTGTATCTGCAAATGAACAGTCTGAGAGCTGAG |
| | | GATACGGCCGTATATTACTGTGCGAAAGTCTCGTACCTTAGCACCGCGTCCTCC |
| | | CTTGACTATTGGGGCCAAGGTACCCTGGTCACCGTCTCGAGTGCCAGCACCAAG |
| | | GGCCCCTCTGTGTTCCCTCTGGCCCCTTCCAGCAAGTCCACCTCTGGCGGAACA |
| | | GCCGCTCTGGGCTGCCTCGTGAAGGACTACTTCCCCGAGCCTGTGACCGTGTCC |
| | | TGGAACTCTGGCGCTCTGACCAGCGGAGTGCACACCTTCCCTGCTGTGCTGCAG |
| | | TCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTG |
| | | GACAAGAAGGTGGGAGGAGGTGGGAGCTTGCATGGCACACGTCAAGAAGAAATG |
| | | ATTGATCACAGACTAACAGACAGAGAATGGGCAGAAGAGTGGAAACATCTTGAC |
| | | CATCTGTTAAACTGCATAATGGACATGGTAGAAAAAACAAGGCGATCTCTCACC |
| | | GTACTAAGGCGGTGTCAAGAAGCAGACCGGGAAGAATTGAATTACTGGATCCGG |
| | | CGGTACAGTGACGCCGAG |
| 40 | Quad 30 | ATGGACATGAGGGTCCCTGCTCAGCTCCTGGGACTCCTGCTGCTCTGGCTCCCA |
| | | GATACCAGATGTGACATCCAGATGACCCAGTCTCCATCCTCCCTGTCTGCATCT |
| | | GTAGGGGACAGAGTCACCATCACTTGTCGGGCAAGTCAGGGCATCAGAAATTAC |
| | | TTAGCCTGGTATCAGCAAAAACCAGGGAAAGCCCCTAAGCTCCTGATCTATGCT |
| | | GCATCCACTTTGCAATCAGGGGTCCCATCTCGGTTCAGTGGCAGTGGATCTGGG |
| | | ACAGATTTCACTCTCACCATCAGCAGCCTACAGCCTGAAGATGTTGCAACTTAT |
| | | TACTGTCAAAGGTATAACCGTGCACCGTATACTTTTGGCCAGGGGACCAAGGTG |
| | | GAAATCAAACGTACGGTGGCCGCTCCCTCCGTGTTCATCTTCCCACCTTCCGAC |
| | | GAGCAGCTGAAGTCCGGCACCGCTTCTGTCGTGTGCCTGCTGAACAACTTCTAC |
| | | CCCCGCGAGGCCAAGGTGCAGTGGAAGGTGGACAACGCCCTGCAGTCCGGCAAC |
| | | TCCCAGGAATCCGTGACCGAGCAGGACTCCAAGGACAGCACCTACTCCCTGTCC |
| | | TCCACCCTGACCCTGTCCAAGGCCGACTACGAGAAGCACAAGGTGTACGCCTGC |
| | | GAAGTGACCCACCAGGGCCTGTCTAGCCCCGTGACCAAGTCTTTCAACCGGGGC |
| | | GAGTGT |
| 41 | Quad 31 | ATGGGATGGTCTTGTATAATTCTGTTCCTGGTGGCAACAGCAACAGGAGTGCAT |
| | | AGCGAGGTCCAACTTGTCGAAAGTGGCGGCGGTTTGGTTCAACCTGGAGGTTCA |
| | | CTTCGACTGTCATGTGCAGCGAGCGGTTATACATTTACGAATTATGGCATGAAT |
| | | TGGGTTAGACAGGCACCAGGAAAGGGACTGGAGTGGGTAGGCTGGATCAATACC |
| | | TACACAGGAGAACCAACGTATGCCGCAGACTTCAAACGACGGTTTACATTTTCC |
| | | TTGGATACCTCTAAGTCTACAGCGTATCTCCAAATGAATTCACTTCGAGCGGAA |
| | | GATACCGCGGTCTACTATTGCGCCAAATACCCTCATTATTATGGGTCATCTCAC |
| | | TGGTATTTCGATGTCTGGGGTCAGGGAACACTGGTAACCGTGTCATCCGCCAGC |
| | | ACCAAGGGCCCCTCTGTGTTCCCTCTGGCCCCTTCCAGCAAGTCCACCTCTGGC |
| | | GGAACAGCCGCTCTGGGCTGCCTCGTGAAGGACTACTTCCCCGAGCCTGTGACC |
| | | GTGTCCTGGAACTCTGGCGCTCTGACCAGCGGAGTGCACACCTTCCCTGCTGTG |
| | | CTGCAGTCCTCCGGCCTGTACTCCCTGTCCTCCGTCGTGACCGTGCCTTCCAGC |
| | | TCTCTGGGCACCCAGACCTACATCTGCAACGTGAACCACAAGCCCTCCAACACC |
| | | AAGGTGGACAAGAAGGTGTTGCATGGCACACGTCAAGAAGAAATGATTGATCAC |
| | | AGACTAACAGACAGAGAATGGGCAGAAGAGTGGAAACATCTTGACCATCTGTTA |
| | | AACTGCATAATGGACATGGTAGAAAAAACAAGGCGATCTCTCACCGTACTAAGG |
| | | CGGTGTCAAGAAGCAGACCGGGAAGAATTGAATTACTGGATCCGGCGGTACAGT |
| | | GACGCCGAG |
| 42 | Quad 32 | ATGGGATGGTCTTGTATAATTCTGTTCCTGGTGGCAACAGCAACAGGAGTGCAT |
| | | AGCGAGGTCCAACTTGTCGAAAGTGGCGGCGGTTTGGTTCAACCTGGAGGTTCA |
| | | CTTCGACTGTCATGTGCAGCGAGCGGTTATACATTTACGAATTATGGCATGAAT |
| | | TGGGTTAGACAGGCACCAGGAAAGGGACTGGAGTGGGTAGGCTGGATCAATACC |
| | | TACACAGGAGAACCAACGTATGCCGCAGACTTCAAACGACGGTTTACATTTTCC |
| | | TTGGATACCTCTAAGTCTACAGCGTATCTCCAAATGAATTCACTTCGAGCGGAA |
| | | GATACCGCGGTCTACTATTGCGCCAAATACCCTCATTATTATGGGTCATCTCAC |
| | | TGGTATTTCGATGTCTGGGGTCAGGGAACACTGGTAACCGTGTCATCCGCCAGC |
| | | ACCAAGGGCCCCTCTGTGTTCCCTCTGGCCCCTTCCAGCAAGTCCACCTCTGGC |
| | | GGAACAGCCGCTCTGGGCTGCCTCGTGAAGGACTACTTCCCCGAGCCTGTGACC |
| | | GTGTCCTGGAACTCTGGCGCTCTGACCAGCGGAGTGCACACCTTCCCTGCTGTG |
| | | CTGCAGTCCTCCGGCCTGTACTCCCTGTCCTCCGTCGTGACCGTGCCTTCCAGC |
| | | TCTCTGGGCACCCAGACCTACATCTGCAACGTGAACCACAAGCCCTCCAACACC |
| | | AAGGTGGACAAGAAGGTGGGAGGAGGTGGGAGCTTGCATGGCACACGTCAAGAA |
| | | GAAATGATTGATCACAGACTAACAGACAGAGAATGGGCAGAAGAGTGGAAACAT |
| | | CTTGACCATCTGTTAAACTGCATAATGGACATCGTAGAAAAAACAAGGCGATCT |
| | | CTCACCGTACTAAGGCGGTGTCAAGAAGCAGACCGGGAAGAATTGAATTACTGG |
| | | ATCCGGCGGTACAGTGACGCCGAG |
| 43 | Quad 33 | ATGGACATGAGGGTCCCTGCTCAGCTCCTGGGGCTCCTGCAGCTCTGGCTCTCA |
| | | GGTGCCAGATGTGACATCCAAATGACCCAGAGTCCTTCCAGCCTCAGTGCGTCA |
| | | GTGGGAGATCGAGTGACGATAACGTGTTCTGCCAGCCAAGACATTTCCAACTAT |
| | | CTTAATTGGTACCAGCAGAAACCGGGAAAGGCCCCGAAAGTGCTCATATACTTT |
| | | ACCAGCAGTCTTCACTCTGGAGTTCCTAGCCGGTTTAGCGGCTCAGGTAGTGGC |
| | | ACCGATTTCACTCTGACCATTAGTTCTCTTCAACCGGAAGATTTTGCAACCTAC |
| | | TATTGTCAGCAGTATTCAACGGTACCTTGGACCTTCGGCCAAGGCACCAAAGTC |
| | | GAGATTAAGCGTACGGTGGCCGCTCCCTCCGTGTTCATCTTCCCACCTTCCGAC |
| | | GAGCAGCTGAAGTCCGGCACCGCTTCTGTCGTGTGCCTGCTGAACAACTTCTAC |
| | | CCCCGCGAGGCCAAGGTGCAGTGGAAGGTGGACAACGCCCTGCAGTCCGGCAAC |
| | | TCCCAGGAATCCGTGACCGAGCAGGACTCCAAGGACAGCACCTACTCCCTGTCC |
| | | TCCACCCTGACCCTGTCCAAGGCCGACTACGAGAAGCACAAGGTGTACGCCTGC |
| | | GAAGTGACCCACCAGGGCCTGTCTAGCCCCGTGACCAAGTCTTTCAACCGGGGC |
| | | GAGTGT |
| 44 | Quad 34 | ATGGATATGCGCGTCCCGGCACAGCTGCTCGGCTTGTTGTTGCTGTGGTTGAGA |
| | | GCTAGGTGCGATATACAGATGACTCAGTCCCCTTCCAGTCTTTCAGCCAGTGTC |
| | | GGCGACCGGGTTACCATTACTTGTCGGGCAAGTCAATCTATAGATAGTTATTTG |
| | | CATTGGTATCAACAAAAACCAGGCAAAGCGCCTAAGTTGTTGATATATTCCGCA |
| | | TCTGAACTGCAATCAGGCGTTCCTTCACGCTTTTCTGGAAGCGGCAGCGGAACC |
| | | GATTTCACTCTTACCATAAGTAGTCTCCAGCCGGAGGATTTTGCTACATACTAT |
| | | TGTCAACAAGTAGTGTGGCGACCGTTCACCTTCGGACAGGGGACAAAAGTAGAA |
| | | ATCAAGCGGGGAGGAGGTGGGAGCTTGCATGGCACACGTCAAGAAGAAATGATT |
| | | GATCACAGACTAACAGACAGAGAATGGGCAGAAGAGTGGAAACATCTTGACCAT |
| | | CTGTTAAACTGCATAATGGACATGGTAGAAAAAACAAGGCGATCTCTCACCGTA |
| | | CTAAGGCGGTGTCAAGAAGCAGACCGGGAAGAATTGAATTACTGGATCCGGCGG |
| | | TACAGTGACGCCGAG |
| 45 | Quad 36 | ATGGAGTTTGGCCTCAGTTGGTTGTTTTTGGTAGCGAAAATTAAAGTACAGTGT |
| | | GAAGTCCAACTCCTGGAGAGCGGGGGGGGTCTGGTACAACCAGGCGGCTCACTG |
| | | CGGCTTAGCTGCGCAGCCTCCGGGTTCACGTTCGCACATGAAACGATGGTGTGG |
| | | GTGCGCCAGGCACCGGGGAAGGGACTCGAATGGGTCTCACATATACCTCCTGAC |
| | | GGTCAGGATCCTTTTTACGCGGACTCTGTGAAGGGACGATTCACAATAAGTAGA |
| | | GACAATAGTAAGAACACCCTTTATTTGCAGATGAACAGTCTGCGGGCGGAAGAT |
| | | ACAGCAGTATATCATTGTGCCCTGCTGCCCAAACGAGGTCCGTGGTTTGACTAT |
| | | TGGGGACAGGGGACTCTCGTTACTGTAAGCTCCGGAGGAGGTGGGAGOTTGCAT |
| | | GGCACACGTCAAGAAGAAATGATTGATCACAGACTAACAGACAGAGAATGGGCA |
| | | GAAGAGTGGAAACATCTTGACCATCTGTTAAACTGCATAATGGACATGGTAGAA |
| | | AAAACAAGGCGATCTCTCACCGTACTAAGGCGGTGTCAAGAAGCAGACCGGGAA |
| | | GAATTGAATTACTGGATCCGGCGGTACAGTGACGCCGAG |
| 46 | Quad 38 | ATGGACATGAGGGTCCCCGCTCAGCTCCTGGGGCTCCTGCTACTCTGGCTCCGA |
| | | GCCAGATGTGATATACAGATGACCCAATCACCAAGCAGCTTGTCCGCTTCAGTG |
| | | GGCGACAGGGTAACTATAACATGCCGCGCAAGCCAATGGATAGGTCCAGAACTC |
| | | TCATGGTACCAACAAAAACCAGGGAAAGCGCCGAAACTGCTTATCTATCACACA |
| | | AGCATTTTGCAATCTGGGGTACCTAGTCGATTCAGTGGCTCTGGCAGTGGGACT |
| | | GACTTTACACTCACCATAAGTTCTCTCCAACCAGAGGACTTTGCAACATACTAT |
| | | TGTCAGCAATATATGTTTCAACCACGCACCTTTGGACAAGGCACAAAAGTTGAA |
| | | ATCCGCGGAGGAGGTGGGAGCTTGCATGGCACACGTCAAGAAGAAATGATTGAT |
| | | CACAGACTAACAGACAGAGAATGGGCAGAAGAGTGGAAACATCTTGACCATCTG |
| | | TTAAACTGCATAATGGACATGGTAGAAAAAACAAGGCGATCTCTCACCGTACTA |
| | | AGGCGGTGTCAAGAAGCAGACCGGGAAGAATTGAATTACTGGATCCGGCGGTAC |
| | | AGTGACGCCGAG |
| 47 | Quad 40 | ATGGACATGAGGGTOCCCGCTCAGCTCCTGGGGCTCCTGCTACTCTGGCTCCGA |
| | | GCCAGATGTGATATTCAAATGACACAGTCACCAACGAGTCTTTCCGCGAGCGTT |
| | | GGGGACCGAGTGACAATAACTTGTCGAGCCTCTCAGTGGATTGGCAACTTGCTG |
| | | GACTGGTATCAGCAAAAGCCGGGAGAAGCCCCGAAGCTGCTCATATACTATGCT |
| | | TCCTTCCTCCAGAGTGGAGTACCTAGCAGATTCAGCGGGGGGGGATTCGGGACT |
| | | GATTTCACTCTTACAATCAGCTCTCTTCAACCCGAGGACTTCGCAACGTACTAC |
| | | TGTCAACAAGCTAACCCTGCGCCGCTTACTTTCGGACAAGGCACTAAGGTCGAA |
| | | ATTAAGCGAGGAGGAGGTGGGAGCTTGCATGGCACACGTCAAGAAGAAATGATT |
| | | GATCACAGACTAACAGACAGAGAATGGGCAGAAGAGTGGAAACATCTTGACCAT |
| | | CTGTTAAACTGCATAATGGACATGGTAGAAAAAACAAGGCGATCTCTCACCGTA |
| | | CTAAGGCGGTGTCAAGAAGCAGACCGGGAAGAATTGAATTACTGGATCCGGCGG |
| | | TACAGTGACGCCGAG |
| 48 | Quad 42 | ATGGACATGAGGGTCCCCGCTCAGCTCCTGGGGCTCCTGCTACTCTGGCTCCGA |
| | | GCCAGATGTGATATACAGATGACCCAATCACCAAGCAGCTTGTCCGCTTCAGTG |
| | | GGCGACAGGGTAACTATAACATGCCGCGCAAGCCAATGGATAGGTCCAGAACTC |
| | | TCATGGTACCAACAAAAACCAGGGAAAGCGCCGAAACTGCTTATCTATCACACA |
| | | AGCATTTTGCAATCTGGGGTACCTAGTCGATTCAGTGGCTCTGGCAGTGGGACT |
| | | GACTTTACACTCACCATAAGTTCTCTCCAACCAGAGGACTTTGCAACATACTAT |
| | | TGTCAGCAATATATGTTTCAACCACGCACCTTTGGACAAGGCACAAAAGTTGAA |
| | | ATCCGCGGAGGAGGTGGGAGCTTGCATGGCACACGTCAAGAAGAAATGATTGAT |
| | | CACAGACTAACAGACAGAGAATGGGCAGAAGAGTGGAAACATCTTGACCATCTG |
| | | TTAAACTGCATAATGGACATGGTAGAAAAAACAAGGCGATCTCTCACCGTACTA |
| | | AGGCGGTGTCAAGAAGCAGACCGGGAAGAATTGAATTACTGGATCCGGCGGTAC |
| | | AGTGACGCCGAGGACTTAAAAGGTGGAGGAGGTAGCGATATTCAAATGACACAG |
| | | TCACCAACGAGTCTTTCCGCGAGCGTTGGGGACCGAGTGACAATAACTTGTCGA |
| | | GCCTCTCAGTGGATTGGCAACTTGCTGGACTGGTATCAGCAAAAGCCGGGAGAA |
| | | GCCCCGAAGCTGCTCATATACTATGCTTCCTTCCTCCAGAGTGGAGTACCTAGC |
| | | AGATTCAGCGGGGGGGGATTCGGGACTGATTTCACTCTTACAATCAGCTCTCTT |
| | | CAACCCGAGGACTTCGCAACGTACTACTGTCAACAAGCTAACCCTGCGCCGCTT |
| | | ACTTTCGGACAAGGCACTAAGGTCGAAATTAAGCGA |
| 49 | Quad 44 | ATGGACATGAGGGTCCCCGCTCAGCTCCTGGGGCTCCTGCTACTCTGGCTCCGA |
| | | GCCAGATGTGACATTCAGATGACTCAGTCACCATCCTCATTGTCTGCATCAGTT |
| | | GGTGACCGAGTTACGATCACATGTCGAGCAAGCCAAAATATAGATTCCAGACTT |
| | | TCATGGTACCAGCAGAAGCCTGGTAAAGCGCCGAAACTCCTCATATATCGCACG |
| | | AGCGTATTGCAATCTGGTGTGCCTTCTCGATTTTCAGGATCTGGGTCTGGCACT |
| | | GACTTCACCTTGACAATATCTTCTCTTCAGCCCGAAGATTTCGCTACCTACTAC |
| | | TGCCAACAATGGGACATGTTTCCTCTGACCTTCGGACAGGGTACAAAGGTCGAG |
| | | ATTAAACGGGGAGGAGGTGGGAGCTTGCATGGCACACGTCAAGAAGAAATGATT |
| | | GATCACAGACTAACAGACAGAGAATGGGCAGAAGAGTGGAAACATCTTGACCAT |
| | | CTGTTAAACTGCATAATGGACATGGTAGAAAAAACAAGGCGATCTCTCACCGTA |
| | | CTAAGGCGGTGTCAAGAAGCAGACCGGGAAGAATTGAATTACTGGATCCGGCGG |
| | | TACAGTGACGCCGAG |
| 50 | Quad 46 | ATGGACATGAGGGTCCCCGCTCAGCTCCTGGGGCTCCTGCTACTCTGGCTCCGA |
| | | GCCAGATGTGACATTCAGATGACTCAGTCACCATCCTCATTGTCTGCATCAGTT |
| | | GGTGACCGAGTTACGATCACATGTCGAGCAAGCCAAAATATAGATTCCAGACTT |
| | | TCATGGTACCAGCAGAAGCCTGGTAAAGCGCCGAAACTCCTCATATATCGCACG |
| | | AGCGTATTGCAATCTGGTGTGCCTTCTCGATTTTCAGGATCTGGGTCTGGCACT |
| | | GACTTCACCTTGACAATATCTTCTCTTCAGCCCGAAGATTTCGCTACCTACTAC |
| | | TGCCAACAATGGGACATGTTTCCTCTGACCTTCGGACAGGGTACAAAGGTCGAG |
| | | ATTAAACGGGGAGGAGGTGGGAGCTTGCATGGCACACGTCAAGAAGAAATGATT |
| | | GATCACAGACTAACAGACAGAGAATGGGCAGAAGAGTGGAAACATCTTGACCAT |
| | | CTCTTAAACTCCATAATGGACATGGTAGAAAAAACAAGGCGATCTCTCACCGTA |
| | | CTAAGGCGGTGTCAAGAAGCAGACCGGGAAGAATTGAATTACTGGATCCGGCGG |
| | | TACACTCACCCCGAGCACTTAAAAGGTGGAGGAGGTAGCGATATACAGATGACT |
| | | CAATCCCCTTCATCCCTCTCAGCTTCCGTAGGGGACAGAGTTACTATAACGTGT |
| | | CGAGCTAGTCAAGACATAGGTGATCGCCTGAGGTGGTATCAGCAAAAACCGGGT |
| | | AAAGCACCTAAACTCCTCATATATCATGGTTCCAGGTTGGAGTCAGGCGTGCCG |
| | | TCACGATTCTCTGGGTCACGCTCTGGCACTGACTTCACATTGACGATTAGTTCT |
| | | CTCCAGCCCGAAGACTTCGCCACCTACTACTGTCAACAGCAATGGTTTCGCCCG |
| | | TATACTTTTGGGCAGGGTACAAAGGTTGAGATTAAACGG |

TABLE 4
Amino acid sequences of binding moieties,
domains and peptides

| SEQ | | | |
|---|---|---|---|
| ID | Domain/ | | Domain/peptide |
| NO: | petptide | Product* | Amino Acid Sequence |
| 51 | Anti-TNF | Humira™ | EVQLVESGGGLVQPGRSLRL |
| | alpha H | | SCAASGFTFDDYAMHVVVRQ |
| | | | APGKGLEVVVSAITWNSGHI |
| | | | DYADSVEGRFTISRDNAKNS |
| | | | LYLQMNSLRAEDTAVYYCAK |
| | | | VSYLSTASSLDYWGQGTLVT |
| | | | VSS |
| 52 | Anti TNF | Humira™ | DIQMTQSPSSLSASVGDRVT |
| | alpha VL | | ITCRASQGIRNYLAVVYQQK |
| | | | PGKAPKLLIYAASTLQSGVP |
| | | | SRFSGSGSGTDFTLTISSLQ |
| | | | PEDVATYYCQRYNRAPYTFG |
| | | | QGTKVEIKR |
| 53 | Anti-CD20 VH | Rituximab | QVQLQQPGAELVKPGASVKM |
| | | | SCKASGYTFTSYNMHWVKQT |
| | | | PGRGLEWIGAIYPGNGDTSY |
| | | | NQKFKGKATLTADKSSSTAY |
| | | | MQLSSLTSEDSAVYYCARST |
| | | | YYGGDVVYFNVWGAGTTVTV |
| | | | SA |
| 54 | Anti-CD20 VL | Rituximab | QIVLSQSPAILSASPGEKVT |
| | | | MTCRASSSVSYIHWFQQKPG |
| | | | SSPKPWIYATSNLASGVPVR |
| | | | FSGSGSGTSYSLTISRVEAE |
| | | | DAATYYCQQVVTSNPPTFGG |
| | | | GTKLEIKR |
| 55 | Anti-VEGF VH | Avastin™ | EVQLVESGGGLVQPGGSLRL |
| | | | SCAASGYTFTNYGMNWVRQA |
| | | | PGKGLEWVGWINTYTGEPTY |
| | | | AADFKRRFTFSLDTSKSTAY |
| | | | LQMNSLRAEDTAVYYCAKYP |
| | | | HYYGSSHVVYFDVWGQGTLV |
| | | | TVSS |
| 56 | Anti-VEGF VL | Avastin™ | DIQMTQSPSSLSASVGDRVT |
| | | | ITCSASQDISNYLNVVYQQK |
| | | | PGKAPKVLIYFTSSLHSGVP |
| | | | SRFSGSGSGTDFTLTISSLQ |
| | | | PEDFATYYCQQYSTVPWTFG |
| | | | QGTKVEIKR |
| 57 | Anti-HER2 VH | Herceptin™ | EVQLVESGGGLVQPGGSLRL |
| | | | SCAASGFTFTDYTMDVVVRQ |
| | | | APGKGLEVVVADVNPNSGGS |
| | | | IYNQRFKGRFTLSVDRSKNT |
| | | | LYLQMNSLRAEDTAVYYCAR |
| | | | NLGPSFYFDYWGQGTLVTVS |
| | | | S |
| 58 | Anti-HER2 VL | Herceptin™ | DIQMTQSPSSLSASVGDRVT |
| | | | ITCKASQDVSIGVAVVYQQK |
| | | | PGKAPKWYSASYRYTGVPSR |
| | | | FSGSGSGTDFTLTISSLQPE |
| | | | DFATYYCQQYYIYPYTFGQG |
| | | | TKVEIKR |
| 59 | Anti-IL6R VH | Actemra™ | EVQLQESGPGLVRPSQTLSL |
| | | | TCTVSGYSITSDHAWSVVVR |
| | | | QPPGRGLEWIGYISYSGITT |
| | | | YNPSLKSRVTMLRDTSKNQF |
| | | | SLRLSSVTAADTAVYYCARS |
| | | | LARTTAMDYWGQGSLVTVSS |
| 60 | Anti-IL6R VL | Actemra™ | DIQMTQSPSSLSASVGDRVT |
| | | | ITCRASQDISSYLNVVYQQK |
| | | | PGKAPKLLIYYTSRLHSGVP |
| | | | SRFSGSGSGTDFTFTISSLQ |
| | | | PEDIATYYCQQGNTLPYTFG |
| | | | QGTKVEIKR |
| 61 | Anti-PD-1 VH | Nivolumab | QVQLVESGGGVVQPGRSLRL |
| | | | DCKASGITFSNSGMHVVVRQ |
| | | | APGKGLEVVVAVIVVYDGSK |
| | | | RYYADSVKGRFTISRDNSKN |
| | | | TLFLQMNSLRAEDTAVYYCA |
| | | | TNDDYWGQGTLVTVSS |
| 62 | Anti-PD-1 VL | Nivolumab | EIVLTQSPATLSLSPGERAT |
| | | | LSCRASQSVSSYLAVVYQQK |
| | | | PGQAPRLLIYDASNRATGIP |
| | | | ARFSGSGSGTDFTLTISSLE |
| | | | PEDFAVYYCQQSSNVVPRTF |
| | | | GQGTKVEIKR |
| 63 | Anti-CTLA4 VH | Ipilimumab | QVQLVESGGGVVQPGRSLRL |
| | | | SCAASGFTFSSYTMHVVVRQ |
| | | | APGKGLEVVVTFISYDGNNK |
| | | | YYADSVKGRFTISRDNSKNT |
| | | | LYLQMNSLRAEDTAIYYCAR |
| | | | TGWLGPFDYWGQGTLVTVSS |
| 64 | Anti-CTLA4 | Ipilimumab | EIVLTQSPGTLSLSPGERAT |
| | VL | | LSCRASQSVGSSYLAVVYQQ |
| | | | KPGQAPRLLIYGAFSRATGI |
| | | | PDRFSGSGSGTDFTLTISRL |
| | | | EPEDFAVYYCQQYGSSPVVT |
| | | | FGQGTKVEIKR |
| 65 | Anti-TNFR1 | DOM1h-131- | EVQLLESGGGLVQPGGSLRL |
| | dAb | 206 | SCAASGFTFAHETMVVVVRQ |
| | | VH | APGKGLEVVVSHIPPDGQDP |
| | | | FYADSVKGRFTISRDNSKNT |
| | | | LYLQMNSLRAEDTAVYHCAL |
| | | | LPKRGPVVFDYWGQGTLVTV |
| | | | SS |
| 66 | Anti-TNFa | TAR 1-5- | DIQMTQSPSSLSASVGDRVT |
| | dAb | 19 Vk | ITCRASQSIDSYLHVVYQQK |
| | | | PGKAPKWYSASELQSGVPSR |
| | | | FSGSGSGTDFTLTISSLQPE |
| | | | DFATYYCQQVVVVRPFTFGQ |
| | | | GTKVEIKR |
| 67 | Anti-VEGF | TAR15-10 | DIQMTQSPSSLSASVGDRVT |
| | dAb | Vk | ITCRASQWIGPELSVVYQQK |
| | | | PGKAPKLLIYHTSILQSGVP |
| | | | SRFSGSGSGTDFTLTISSLQ |
| | | | PEDFATYYCQQYMFQPRTFG |
| | | | QGTKVEIR |
| 68 | Anti-EGFR | DOM16-39- | DIQMTQSPTSLSASVGDRVT |
| | dAb | 109 Vk | ITCRASQWIGNLLDVVYQQK |
| | | | PGEAPKLLIYYASFLQSGVP |
| | | | SRFSGGGFGTDFTLTISSLQ |
| | | | PEDFATYYCQQANPAPLTFG |
| | | | QGTKVEIKR |
| 69 | Anti-CD38 | DOM | DIQMTQSPSSLSASVGDRVT |
| | dAb | 11-3 Vk | ITCRASQNIDSRLSVVYQQK |
| | | | PGKAPKWYRTSVLQSGVPSR |
| | | | FSGSGSGTDFTLTISSLQPE |
| | | | DFATYYCQQVVDMFPLTFGQ |
| | | | GTKVEIKR |
| 70 | Anti-CD138 | DOM 12- | DIQMTQSPSSLSASVGDRVT |
| | dAb | 45 Vk | ITCRASQDIGDRLRVVYQQK |
| | | | PGKAPKLLIYHGSRLESGVP |
| | | | SRFSGSRSGTDFTLTISSLQ |
| | | | PEDFATYYCQQQVVFRPYTF |
| | | | GQGTKVEIKR |
| 71 | Anti-NY- | | KQEVTQIPAALSVPEGENLV |
| | ESO-1 Vα | | LNCSFTDSAIYNLQWFRQDP |
| | | | GKGLTSLLLITPWQREQTSG |
| | | | RLNASLDKSSGRSTLYIAAS |
| | | | QPGDSATYLCAVRPLLDGTY |
| | | | IPTFGRGTSLIVHPY |
| 72 | Anti-NY- | | NAGVTQTPKFQVLKTGQSMT |
| | ESO-1 Vβ | | LQCAQDMNHEYMSVVYRQDP |
| | | | GMGLRLIHYSVAIQTDQGEV |
| | | | PNGYNVSRSTIEDFPLRLLS |
| | | | AAPSQTSVYFCASSYLGNTG |
| | | | ELFFGEGSRLTVL |
| 73 | IL2 | Human IL2 | APTSSSTKKTQLQLEHLLLD |
| | | | LQMILNGINNYKNPKLTRML |
| | | | TFKFYMPKKATELKHLQCLE |
| | | | EELKPLEEVLNLAQSKNFHL |
| | | | RPRDLISNINVIVLELKGSE |
| | | | TTFMCEYADETATIVEFLNR |
| | | | WITFCQSIISTLT |
| 74 | Anti-GFP VH | Nanobody™ | QVQLVESGGALVQPGGSLRL |
| | | | SCAASGFPVNRYSMRVVYRQ |
| | | | APGKEREVVVAGMSSAGDRS |
| | | | SYEDSVKGRFTISRDDARNT |
| | | | VYLQMNSLKPEDTAVYYCNV |
| | | | NVGFEYWGQGTQVTVSS |
| 75 | Anti-IL1R1 | DOM4-122- | DIQMTQSPSSLSASVGDRVT |
| | | 23 Vk | ITCRASQSIIKHLKVVYQQK |
| | | | PGKAPKLLIYGASRLQSGVP |
| | | | SRFSGSGSGTDFTLTISSLQ |
| | | | PEDFATYYCQQGARWPQTFG |
| | | | QGTKVEIKR |
| 76 | FLT1 | EYLEA™ | SDTGRPFVEMYSEIPEIIHM |
| | | | TEGRELVIPCRVTSPNITVT |
| | | | LKKFPLDTLIPDGKRIIWDS |
| | | | RKGFIISNATYKEIGLLTCE |
| | | | ATVNGHLYKTNYLTHRQTNT |
| | | | IIDV |
| 77 | KDR | EYLEA™ | VLSPSHGIELSVGEKLVLNC |
| | | | TARTELNVGIDFNVVEYPSS |
| | | | KHQHKKLVNRDLKTQSGSEM |
| | | | KKFLSTLTIDGVTRSDQGLY |
| | | | TCAASSGLMTKKNSTFVRVH |
| | | | EK |
| 78 | Aflibercept | | SDTGRPFVEMYSEIPEIIHM |
| | | | TEGRELVIPCRVTSPNITVT |
| | | | LKKFPLDTLIPDGKRIIWDS |
| | | | RKGFIISNATYKEIGLLTCE |
| | | | ATVNGHLYKTNYLTHRQTNT |
| | | | IIDVVLSPSHGIELSVGEKL |
| | | | VLNCTARTELNVGIDFNWEY |
| | | | PSSKHQHKKLVNRDLKTQSG |
| | | | SEMKKFLSTLTIDGVTRSDQ |
| | | | GLYTCAASSGLMTKKNSTFV |
| | | | RVHEKDKTHTCPPCPAPELL |
| | | | GGPSVFLFPPKPKDTLMISR |
| | | | TPEVTCVVVDVSHEDPEVKF |
| | | | NVVYVDGVEVHNAKTKPREE |
| | | | QYNSTYRVVSVLTVLHQDVV |
| | | | LNGKEYKCKVSNKALPAPIE |
| | | | KTISKAKGQPREPQVYTLPP |
| | | | SRDELTKNQVSLTCLVKGFY |
| | | | PSDIAVEWESNGQPENNYKT |
| | | | TPPVLDSDGSFFLYSKLTVD |
| | | | KSRWQQGNVFSCSVMHEALH |
| | | | NHYTQKSLSLSPG |
| 79 | GLP-1(7-37)- | | HAPGTFTSDVSSYLEGQAAK |
| | Pro9 | | EFIAWLVKGRG |
| 80 | Peptide YY | | IKPEAPGEDASPEELNRYYA |
| | | | SLRHYLNLVTRQRY |
| 81 | EXENDIN-4 | | HGEGTFTSDLSKQMEEEAVR |
| | | | LFIEWLKNGGPSSGAPPPSH |
| | | | GEGTFTSDVSSYLEEQAAKE |
| | | | FIAINLVKGGGGGGGSGGGG |
| | | | SGGGGSAESKYGPPCPPCPA |
| | | | PEAAGGPSVFLFPPKPKDTL |
| | | | MISRTPEVICVVVDVSQEDP |
| | | | EVQFNWYVDGVEVHNAKTKP |
| 82 | DURAGLUTIDE™ | | REEQFNSTYRVVSVLTVLHQ |
| | | | DVVLNGKEYKCKVSNKGLPS |
| | | | SIEKTISKAKGQPREPQVYT |
| | | | LPPSQEEMTKNQVSLTCLVK |
| | | | GFYPSDIAVEWESNGQPENN |
| | | | YKTTPPVLDSDGSFFLYSRL |
| | | | TVDKSRWQEGNVFSCSVMHE |
| | | | ALHNHYTQKSLSLSLG |

*indicates product comprising the domain.

TABLE 5
Amino acid sequences of Quad
polypeptides

| SEQ | | |
|---|---|---|
| NO: | | AMINO |
| ID | POLYPEPTIDE | ACID SEQUENCE |
| 83 | Quad 1 | NAGVTQTPKFQVLKTGQSMT |
| | | LQCAQDMNHEYMSWYRQDPG |
| | | MGLRLIHYSVAIQTTDQGEV |
| | | PNGYNVSRSTIEDFPLRLLS |
| | | AAPSQTSVYFCASSYLGNTG |
| | | ELFFGEGSRLTVLEDLKNVF |
| | | PPEVAVFEPSEAEISHTQKA |
| | | TLVCLATGFYPDHVELSWWV |
| | | NGKEVHSGVSTDPQPLKEQP |
| | | ALNDSRYALSSRLRVSATFW |
| | | QDPRNHFRCQVQFYGLSEND |
| | | EWTQDRAKPVTQIVSAEAWG |
| | | RADASTKGPSVFPLAPSSKS |
| | | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VLTDREWAEEWKHLDHLLNC |
| | | IMDMVEKTRRSLTVLRRCQE |
| | | ADREELNYWIRRYSDAE |
| 84 | Quad 2 | NAGVTQTPKFQVLKTGQSMT |
| | | LQCAQDMNHEYMSWYRQDPG |
| | | MGLRLIHYSVAIQTTDQGEV |
| | | PNGYNVSRSTIEDFPLRLLS |
| | | AAPSQTSVYFCASSYLGNTG |
| | | ELFFGEGSRLTVLEDLKNVF |
| | | PPEVAVFEPSEAEISHTQKA |
| | | TLVCLATGFYPDHVELSWWV |
| | | NGKEVHSGVSTDPQPLKEQP |
| | | ALNDSRYALSSRLRVSATFW |
| | | QDPRNHFRCQVQFYGLSEND |
| | | EWTQDRAKPVTQIVSAEAWG |
| | | RADASTKGPSVFPLAPSSKS |
| | | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VGGGGSLTDREWAEEWKHLD |
| | | HLLNCIMDMVEKTRRSLTVL |
| | | RRCQEADREELNYWIRRYSD |
| | | AE |
| 85 | Quad 3 | NAGVTQTPKFQVLKTGQSMT |
| | | LQCAQDMNHEYMSWYRQDPG |
| | | MGLRLIHYSVAIQTTDQGEV |
| | | PNGYNVSRSTIEDFPLRLLS |
| | | AAPSQTSVYFCASSYLGNTG |
| | | ELFFGEGSRLTVLEDLKNVF |
| | | PPEVAVFEPSEAEISHTQKA |
| | | TLVCLATGFYPDHVELSWWV |
| | | NGKEVHSGVCTDPQPLKEQP |
| | | ALNDSRYALSSRLRVSATFW |
| | | QDPRNHFRCQVQFYGLSEND |
| | | EWTQDRAKPVTQIVSAEAWG |
| | | RADASTKGPSVFPLAPSSKS |
| | | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VLTDREWAEEWKHLDHLLNC |
| | | IMDMVEKTRRSLTVLRRCQE |
| | | ADREELNYWIRRYSDAE |
| 86 | Quad 4 | NAGVTQTPKFQVLKTGQSMT |
| | | LQCAQDMNHEYMSWYRQDPG |
| | | MGLRLIHYSVAIQTTDQGEV |
| | | PNGYNVSRSTIEDFPLRLLS |
| | | AAPSQTSVYFCASSYLGNTG |
| | | ELFFGEGSRLTVLEDLKNVF |
| | | PPEVAVFEPSEAEISHTQKA |
| | | TLVCLATGFYPDHVELSWWV |
| | | NGKEVHSGVCTDPQPLKEQP |
| | | ALNDSRYALSSRLRVSATFW |
| | | QDPRNHFRCQVQFYGLSEND |
| | | EWTQDRAKPVTQIVSAEAWG |
| | | RADASTKGPSVFPLAPSSKS |
| | | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VGGGGSLTDREWAEEWKHLD |
| | | HLLNCIMDMVEKTRRSLTVL |
| | | RRCQEADREELNYWIRRYSD |
| | | AE |
| 87 | Quad 5 | NAGVTQTPKFQVLKTGQSMT |
| | | LQCAQDMNHEYMSWYRQDPG |
| | | MGLRLIHYSVAIQTTDQGEV |
| | | PNGYNVSRSTIEDFPLRLLS |
| | | AAPSQTSVYFCASSYLGNTG |
| | | ELFFGEGSRLTVLEDLKNVF |
| | | PPEVAVFEPSEAEISHTQKA |
| | | TLVCLATGFYPDHVELSWWV |
| | | NGKEVHSGVCTDPQPLKEQP |
| | | ALNDSRYALSSRLRVSATFW |
| | | QDPRNHFRCQVQFYGLSEND |
| | | EWTQDRAKPVTQIVSAEAWG |
| | | RADLTDREWAEEWKHLDHLL |
| | | NCIMDMVEKTRRSLTVLRRC |
| | | QEADREELNYWIRRYSDAE |
| 88 | Quad 6 | NAGVTQTPKFQVLKTGQSMT |
| | | LQCAQDMNHEYMSWYRQDPG |
| | | MGLRLIHYSVAIQTTDQGEV |
| | | PNGYNVSRSTIEDFPLRLLS |
| | | AAPSQTSVYFCASSYLGNTG |
| | | ELFFGEGSRLTVLEDLKNVF |
| | | PPEVAVFEPSEAEISHTQKA |
| | | TLVCLATGFYPDHVELSWWV |
| | | NGKEVHSGVCTDPQPLKEQP |
| | | ALNDSRYALSSRLRVSATFW |
| | | QDPRNHFRCQVQFYGLSEND |
| | | EWTQDRAKPVTQIVSAEAWG |
| | | RADGGGGSLTDREWAEEWKH |
| | | LDHLLNCIMDMVEKTRRSLT |
| | | VLRRCQEADREELNYWIRRY |
| | | SDAE |
| 89 | Quad 7 | KQEVTQIPAALSVPEGENLV |
| | | LNCSFTDSAIYNLQWFRQDP |
| | | GKGLTSLLLITPWQREQTSG |
| | | RLNASLDKSSGRSTLYIAAS |
| | | QPGDSATYLCAVRPLLDGTY |
| | | IPTEGRGTSLIVHPYIQNPD |
| | | PAVYQLRDSKSSDKSVCLFT |
| | | DFDSQTNVSQSKDSDVYITD |
| | | KTVLDMRSMDEKSNSAVAWS |
| | | NKSDFACANAENNSIIPEDT |
| | | FFPSPESSTVAAPSVFIEPP |
| | | SDEQLKSGTASVVCLLNNEY |
| | | PREAKVQWKVDNALQSGNSQ |
| | | ESVTEQDSKDSTYSLSSTLT |
| | | LSKADYEKHKVYACEVTHQG |
| | | LSSPVTKSFNRGEC |
| 90 | Quad 8 | KQEVTQIPAALSVPEGENLV |
| | | LNCSFTDSAIYNLQWFRQDP |
| | | GKGLTSLLLITPWQREQTSG |
| | | RLNASLDKSSGRSTLYIAAS |
| | | QPGDSATYLCAVRPLLDGTY |
| | | IPTEGRGTSLIVHPYIQNPD |
| | | PAVYQLRDSKSSDKSVCLFT |
| | | DFDSQTNVSQSKDSDVYITD |
| | | KCVLDMRSMDEKSNSAVAWS |
| | | NKSDFACANAENNSIIPEDT |
| | | FFPSPESSTVAAPSVFIEPP |
| | | SDEQLKSGTASVVCLLNNEY |
| | | PREAKVQWKVDNALQSGNSQ |
| | | ESVTEQDSKDSTYSLSSTLT |
| | | LSKADYEKHKVYACEVTHQG |
| | | LSSPVTKSFNRGEC |
| 91 | Quad 9 | KQEVTQIPAALSVPEGENLV |
| | | LNCSFTDSAIYNLQWFRQDP |
| | | GKGLTSLLLITPWQREQTSG |
| | | RLNASLDKSSGRSTLYIAAS |
| | | QPGDSATYLCAVRPLLDGTY |
| | | IPTFGRGTSLIVHPYIQNPD |
| | | PAVYQLRDSKSSDKSVCLFT |
| | | DFDSQTNVSQSKDSDVYITD |
| | | KCVLDMRSMDEKSNSAVAWS |
| | | NKSDFACANAENNSIIPEDT |
| | | FFPSPESS |
| 92 | Quad 10 | NAGVTQTPKFQVLKTGQSMT |
| | | LQCAQDMNHEYMSWYRQDPG |
| | | MGLRLIHYSVAIQTTDQGEV |
| | | PNGYNVSRSTIEDFPLRLLS |
| | | AAPSQTSVYFCASSYLGNTG |
| | | ELFFGEGSRLTVLEDLKNVF |
| | | PPEVAVFEPSEAEISHTQKA |
| | | TLVCLATGFYPDHVELSWWV |
| | | NGKEVHSGVSTDPQPLKEQP |
| | | ALNDSRYALSSRLRVSATFW |
| | | QDPRNHFRCQVQFYGLSEND |
| | | EWTQDRAKPVTQIVSAEAWG |
| | | RADASTKGPSVFPLAPSSKS |
| | | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VLTDREWAEEWKHLDHLLNC |
| | | IMDMVEKTRRSLTVLRRCQE |
| | | ADREELNYWIRRYSDAEAPT |
| | | SSSTKKTQLQLEHLLLDLQM |
| | | ILNGINNYKNPKLTRMLTFK |
| | | FYMPKKATELKHLQCLEEEL |
| | | KPLEEVLNLAQSKNFHLRPR |
| | | DLISNINVIVLELKGSETTF |
| | | MCEYADETATIVEFLNRWIT |
| | | FCQSIISTLT |
| 93 | Quad 11 | NAGVTQTPKFQVLKTGQSMT |
| | | LQCAQDMNHEYMSWYRQDPG |
| | | MGLRLIHYSVAIQTTDQGEV |
| | | PNGYNVSRSTIEDFPLRLLS |
| | | AAPSQTSVYFCASSYLGNTG |
| | | ELFFGEGSRLTVLEDLKNVF |
| | | PPEVAVFEPSEAEISHTQKA |
| | | TLVCLATGFYPDHVELSWWV |
| | | NGKEVHSGVSTDPQPLKEQP |
| | | ALNDSRYALSSRLRVSATFW |
| | | QDPRNHFRCQVQFYGLSEND |
| | | EWTQDRAKPVTQIVSAEAWG |
| | | RADASTKGPSVFPLAPSSKS |
| | | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VGGGGSLTDREWAEEWKHLD |
| | | HLLNCIMDMVEKTRRSLTVL |
| | | RRCQEADREELNYWIRRYSD |
| | | AEAPTSSSTKKTQLQLEHLL |
| | | LDLQMILNGINNYKNPKLTR |
| | | MLTFKFYMPKKATELKHLQC |
| | | LEEELKPLEEVLNLAQSKNF |
| | | HLRPRDLISNINVIVLELKG |
| | | SETTFMCEYADETATIVEFL |
| | | NRWITFCQSIISTLT |
| 94 | Quad 12 | NAGVTQTPKFQVLKTGQSMT |
| | | LQCAQDMNHEYMSWYRQDPG |
| | | MGLRLIHYSVAIQTTDQGEV |
| | | PNGYNVSRSTIEDFPLRLLS |
| | | AAPSQTSVYFCASSYLGNTG |
| | | ELFFGEGSRLTVLEDLKNVF |
| | | PPEVAVFEPSEAEISHTQKA |
| | | TLVCLATGFYPDHVELSWWV |
| | | NGKEVHSGVSTDPQPLKEQP |
| | | ALNDSRYALSSRLRVSATFW |
| | | QDPRNHFRCQVQFYGLSEND |
| | | EWTQDRAKPVTQIVSAEAWG |
| | | RADASTKGPSVFPLAPSSKS |
| | | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VEPKSCDKTHTCPPCPLTDR |
| | | EWAEEWKHLDHLLNCIMDMV |
| | | EKTRRSLTVLRRCQEADREE |
| | | LNYWIRRYSDAE |
| 95 | Quad 13 | NAGVTQTPKFQVLKTGQSMTL |
| | | QCAQDMNHEYMSWYRQDPGM |
| | | GLRLIHYSVAIQTTDQGEVP |
| | | NGYNVSRSTIEDFPLRLLSA |
| | | APSQTSVYFCASSYLGNTGE |
| | | LFFGEGSRLTVLEDLKNVFP |
| | | PEVAVFEPSEAEISHTQKAT |
| | | LVCLATGFYPDHVELSWWVN |
| | | GKEVHSGVSTDPQPLKEQPA |
| | | LNDSRYALSSRLRVSATFWQ |
| | | DPRNHFRCQVQFYGLSENDE |
| | | WTQDRAKPVTQIVSAEAWGR |
| | | ADASTKGPSVFPLAPSSKST |
| | | SGGTAALGCLVKDYFPEPVT |
| | | VSWNSGALTSGVHTFPAVLQ |
| | | SSGLYSLSSVVTVPSSSLGT |
| | | QTYICNVNHKPSNTKVDKKV |
| | | EPKSCDKTHTCPPCPGGGGS |
| | | LTDREWAEEWKHLDHLLNCI |
| | | MDMVEKTRRSLTVLRRCQEA |
| | | DREELNYWIRRYSDAE |
| 96 | Quad 14 | QVQLVESGGALVQPGGSLRL |
| | | SCAASGFPVNRYSMRWYRQA |
| | | PGKEREWVAGMSSAGDRSSY |
| | | EDSVKGRFTISRDDARNTVY |
| | | LQMNSLKPEDTAVYYCNVNV |
| | | GFEYWGQGTQVTVSSKLTDR |
| | | EWAEEWKHLDHLLNCIMDMV |
| | | EKTRRSLTVLRRCQEADREE |
| | | LNYWIRRYSDAE |
| 97 | Quad15 | QVQLVESGGALVQPGGSLRL |
| | | SCAASGFPVNRYSMRWYRQA |
| | | PGKEREWVAGMSSAGDRSSY |
| | | EDSVKGRFTISRDDARNTVY |
| | | LQMNSLKPEDTAVYYCNVNV |
| | | GFEYWGQGTQVTVSSKGGGG |
| | | SLTDREWAEEWKHLDHLLNC |
| | | IMDMVEKTRRSLTVLRRCQE |
| | | ADREELNYWIRRYSDAE |
| 98 | Quad 18 | QVQLVESGGALVQPGGSLRL |
| | | SCAASGFPVNRYSMRWYRQA |
| | | PGKEREWVAGMSSAGDRSSY |
| | | EDSVKGRFTISRDDARNTVY |
| | | LQMNSLKPEDTAVYYCNVNV |
| | | GFEYWGQGTQVTVSSKLTDR |
| | | EWAEEWKHLDHLLNCIMDMV |
| | | EKTRRSLTVLRRCQEADREE |
| | | LNYWIRRYSDAEAPTSSSTK |
| | | KTQLQLEHLLLDLQMILNGI |
| | | NNYKNPKLTRMLTFKFYMPK |
| | | KATELKHLQCLEEELKPLEE |
| | | VLNLAQSKNFHLRPRDLISN |
| | | INVIVLELKGSETTFMCEYA |
| | | DETATIVEFLNRWITFCQSI |
| | | ISTLT |
| 99 | Quad 19 | QVQLVESGGALVQPGGSLRL |
| | | SCAASGFPVNRYSMRWYRQA |
| | | PGKEREWVAGMSSAGDRSSY |
| | | EDSVKGRFTISRDDARNTVY |
| | | LQMNSLKPEDTAVYYCNVNV |
| | | GFEYWGQGTQVTVSSKGGGG |
| | | SLTDREWAEEWKHLDHLLNC |
| | | IMDMVEKTRRSLTVLRRCQE |
| | | ADREELNYWIRRYSDAEAPT |
| | | SSSTKKTQLQLEHLLLDLQM |
| | | ILNGINNYKNPKLTRMLTFK |
| | | FYMPKKATELKHLQCLEEEL |
| | | KPLEEVLNLAQSKNFHLRPR |
| | | DLISNINVIVLELKGSETTF |
| | | MCEYADETATIVEFLNRWIT |
| | | FCQSIISTLT |
| 100 | Quad 20 | KQEVTQIPAALSVPEGENLV |
| | | LNCSFTDSAIYNLQWFRQDP |
| | | GKGLTSLLLITPWQREQTSG |
| | | RLNASLDKSSGRSTLYIAAS |
| | | QPGDSATYLCAVRPLLDGTY |
| | | IPTEGRGTSLIVHPYIQNPD |
| | | PAVYQLRDSKSSDKSVCLFT |
| | | DFDSQTNVSQSKDSDVYITD |
| | | KTVLDMRSMDEKSNSAVAWS |
| | | NKSDFACANAENNSIIPEDT |
| | | FFPSPESSASTKGPSVFPLA |
| | | PSSKSTSGGTAALGCLVKDY |
| | | FPEPVTVSWNSGALTSGVHT |
| | | FPAVLQSSGLYSLSSVVTVP |
| | | SSSLGTQTYICNVNHKPSNT |
| | | KVDKKVLHGTRQEEMIDHRL |
| | | TDREWAEEWKHLDHLLNCIM |
| | | DMVEKTRRSLTVLRRCQEAD |
| | | REELNYWIRRYSDAEDLK |
| 101 | Quad 21 | KQEVTQIPAALSVPEGENLV |
| | | LNCSFTDSAIYNLQWFRQDP |
| | | GKGLTSLLLITPWQREQTSG |
| | | RLNASLDKSSGRSTLYIAAS |
| | | QPGDSATYLCAVRPLLDGTY |
| | | IPTEGRGTSLIVHPYIQNPD |
| | | PAVYQLRDSKSSDKSVCLFT |
| | | DFDSQTNVSQSKDSDVYITD |
| | | KTVLDMRSMDEKSNSAVAWS |
| | | NKSDFACANAENNSIIPEDT |
| | | FFPSPESSASTKGPSVFPLA |
| | | PSSKSTSGGTAALGCLVKDY |
| | | FPEPVTVSWNSGALTSGVHT |
| | | FPAVLQSSGLYSLSSVVTVP |
| | | SSSLGTQTYICNVNHKPSNT |
| | | KVDKKVGGGGSLHGTRQEEM |
| | | IDHRLTDREWAEEWKHLDHL |
| | | LNCIMDMVEKTRRSLTVLRR |
| | | CQEADREELNYWIRRYSDAE |
| | | DLK |
| 102 | Quad 22 | NAGVTQTPKFQVLKTGQSMT |
| | | LQCAQDMNHEYMSWYRQDPG |
| | | MGLRLIHYSVAIQTTDQGEV |
| | | PNGYNVSRSTIEDFPLRLLS |
| | | AAPSQTSVYFCASSYLGNTG |
| | | ELFFGEGSRLTVLEDLKNVF |
| | | PPEVAVFEPSEAEISHTQKA |
| | | TLVCLATGFYPDHVELSWWV |
| | | NGKEVHSGVSTDPQPLKEQP |
| | | ALNDSRYALSSRLRVSATFW |
| | | QDPRNHFRCQVQFYGLSEND |
| | | EWTQDRAKPVTQIVSAEAWG |
| | | RADTVAAPSVFIFPPSDEQL |
| | | KSGTASVVCLLNNFYPREAK |
| | | VQWKVDNALQSGNSQESVTE |
| | | QDSKDSTYSLSSTLTLSKAD |
| | | YEKHKVYACEVTHQGLSSPV |
| | | TKSFNRGEC |
| 103 | Quad 28 | EVQLVESGGGLVQPGRSLRL |
| | | SCAASGETFDDYAMHWVRQA |
| | | PGKGLEWVSAITWNSGHIDY |
| | | ADSVEGRFTISRDNAKNSLY |
| | | LQMNSLRAEDTAVYYCAKVS |
| | | YLSTASSLDYWGQGTLVTVS |
| | | SASTKGPSVFPLAPSSKSTS |
| | | GGTAALGCLVKDYFPEPVTV |
| | | SWNSGALTSGVHTFPAVLQS |
| | | SGLYSLSSVVTVPSSSLGTQ |
| | | TYICNVNHKPSNTKVDKKVL |
| | | HGTRQEEMIDHRLTDREWAE |
| | | EWKHLDHLLNCIMDMVEKTR |
| | | RSLTVLRRCQEADREELNYW |
| | | IRRYSDAEDLK |
| 104 | Quad 29 | EVQLVESGGGLVQPGRSLRL |
| | | SCAASGETFDDYAMHWVRQA |
| | | PGKGLEWVSAITWNSGHIDY |
| | | ADSVEGRFTISRDNAKNSLY |
| | | LQMNSLRAEDTAVYYCAKVS |
| | | YLSTASSLDYWGQGTLVTVS |
| | | SASTKGPSVFPLAPSSKSTS |
| | | GGTAALGCLVKDYFPEPVTV |
| | | SWNSGALTSGVHTFPAVLQS |
| | | SGLYSLSSVVTVPSSSLGTQ |
| | | TYICNVNHKPSNTKVDKKVG |
| | | GGGSLHGTRQEEMIDHRLTD |
| | | REWAEEWKHLDHLLNCIMDM |
| | | VEKTRRSLTVLRRCQEADRE |
| | | ELNYWIRRYSDAEDLK |
| 105 | Quad 30 | DIQMTQSPSSLSASVGDRVT |
| | | ITCRASQGIRNYLAWYQQKP |
| | | GKAPKLLIYAASTLQSGVPS |
| | | RFSGSGSGTDFTLTISSLQP |
| | | EDVATYYCQRYNRAPYTFGQ |
| | | GTKVEIKRTVAAPSVFIFPP |
| | | SDEQLKSGTASVVCLLNNFY |
| | | PREAKVQWKVDNALQSGNSQ |
| | | ESVTEQDSKDSTYSLSSTLT |
| | | LSKADYEKHKVYACEVTHQG |
| | | LSSPVTKSFNRGEC |
| 106 | Quad 31 | EVQLVESGGGLVQPGGSLRL |
| | | SCAASGYTFTNYGMNWVRQA |
| | | PGKGLEWVGWINTYTGEPTY |
| | | AADFKRRFTFSLDTSKSTAY |
| | | LQMNSLRAEDTAVYYCAKYP |
| | | HYYGSSHWYFDVWGQGTLVT |
| | | VSSASTKGPSVFPLAPSSKS |
| | | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VLHGTRQEEMIDHRLTDREW |
| | | AEEWKHLDHLLNCIMDMVEK |
| | | TRRSLTVLRRCQEADREELN |
| | | YWIRRYSDAEDLK |
| 107 | Quad 32 | EVQLVESGGGLVQPGGSLRL |
| | | SCAASGYTFTNYGMNWVRQA |
| | | PGKGLEWVGWINTYTGEPTY |
| | | AADFKRRFTFSLDTSKSTAY |
| | | LQMNSLRAEDTAVYYCAKYP |
| | | HYYGSSHWYFDVWGQGTLVT |
| | | VSSASTKGPSVFPLAPSSKS |
| | | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VGGGGSLHGTRQEEMIDHRL |
| | | TDREWAEEWKHLDHLLNCIM |
| | | DMVEKTRRSLTVLRRCQEAD |
| | | REELNYWIRRYSDAEDLK |
| 108 | Quad 33 | DIQMTQSPSSLSASVGDRVT |
| | | ITCSASQDISNYLNWYQQKP |
| | | GKAPKVLIYFTSSLHSGVPS |
| | | RFSGSGSGTDFTLTISSLQP |
| | | EDFATYYCQQYSTVPWTFGQ |
| | | GTKVEIKRTVAAPSVFIFPP |
| | | SDEQLKSGTASVVCLLNNFY |
| | | PREAKVQWKVDNALQSGNSQ |
| | | ESVTEQDSKDSTYSLSSTLT |
| | | LSKADYEKHKVYACEVTHQG |
| | | LSSPVTKSFNRGEC |
| 109 | Quad 34 | DIQMTQSPSSLSASVGDRVT |
| | | ITCRASQSIDSYLHWYQQKP |
| | | GKAPKLLIYSASELQSGVPS |
| | | RFSGSGSGTDFTLTISSLQP |
| | | EDFATYYCQQVVWRPFTFGQ |
| | | GTKVEIKRGGGGSLHGTRQE |
| | | EMIDHRLTDREWAEEWKHLD |
| | | HLLNCIMDMVEKTRRSLTVL |
| | | RRCQEADREELNYWIRRYSD |
| | | AEDLK |
| 110 | Quad 36 | EVQLLESGGGLVQPGGSLRL |
| | | SCAASGETFAHETMVWVRQA |
| | | PGKGLEWVSHIPPDGQDPFY |
| | | ADSVKGRFTISRDNSKNTLY |
| | | LQMNSLRAEDTAVYHCALLP |
| | | KRGPWFDYWGQGTLVTVSSG |
| | | GGGSLHGTRQEEMIDHRLTD |
| | | REWAEEWKHLDHLLNCIMDM |
| | | VEKTRRSLTVLRRCQEADRE |
| | | ELNYWIRRYSDAEDLK |
| 111 | Quad 38 | DIQMTQSPSSLSASVGDRVT |
| | | ITCRASQWIGPELSWYQQKP |
| | | GKAPKLLIYHTSILQSGVPS |
| | | RFSGSGSGTDFTLTISSLQP |
| | | EDFATYYCQQYMFQPRTFGQ |
| | | GTKVEIRGGGGSLHGTRQEE |
| | | MIDHRLTDREWAEEWKHLDH |
| | | LLNCIMDMVEKTRRSLTVLR |
| | | RCQEADREELNYWIRRYSDA |
| | | EDLK |
| 112 | Quad 40 | DIQMTQSPTSLSASVGDRVT |
| | | ITCRASQWIGNLLDWYQQKP |
| | | GEAPKLLIYYASFLQSGVPS |
| | | RFSGGGEGTDFTLTISSLQP |
| | | EDFATYYCQQANPAPLTFGQ |
| | | GTKVEIKRGGGGSLHGTRQE |
| | | EMIDHRLTDREWAEEWKHLD |
| | | HLLNCIMDMVEKTRRSLTVL |
| | | RRCQEADREELNYWIRRYSD |
| | | AEDLK |
| 113 | Quad 42 | DIQMTQSPSSLSASVGDRVT |
| | | ITCRASQWIGPELSWYQQKP |
| | | GKAPKLLIYHTSILQSGVPS |
| | | RFSGSGSGTDFTLTISSLQP |
| | | EDFATYYCQQYMFQPRTFGQ |
| | | GTKVEIRGGGGSLHGTRQEE |
| | | MIDHRLTDREWAEEWKHLDH |
| | | LLNCIMDMVEKTRRSLTVLR |
| | | RCQEADREELNYWIRRYSDA |
| | | EDLKGGGGSDIQMTQSPTSL |
| | | SASVGDRVTITCRASQWIGN |
| | | LLDWYQQKPGEAPKLLIYYA |
| | | SFLQSGVPSRFSGGGEGTDF |
| | | TLTISSLQPEDFATYYCQQA |
| | | NPAPLTFGQGTKVEIKR |
| 114 | Quad 44 | DIQMTQSPSSLSASVGDRVT |
| | | ITCRASQNIDSRLSWYQQKP |
| | | GKAPKLLIYRTSVLQSGVPS |
| | | RFSGSGSGTDFTLTISSLQP |
| | | EDFATYYCQQWDMFPLTFGQ |
| | | GTKVEIKRGGGGSLHGTRQE |
| | | EMIDHRLTDREWAEEWKHLD |
| | | HLLNCIMDMVEKTRRSLTVL |
| | | RRCQEADREELNYWIRRYSD |
| | | AEDLK |
| 115 | Quad 46 | DIQMTQSPSSLSASVGDRVT |
| | | ITCRASQNIDSRLSWYQQKP |
| | | GKAPKLLIYRTSVLQSGVPS |
| | | RFSGSGSGTDFTLTISSLQP |
| | | EDFATYYCQQWDMFPLTFGQ |
| | | GTKVEIKRGGGGSLHGTRQE |
| | | EMIDHRLTDREWAEEWKHLD |
| | | HLLNCIMDMVEKTRRSLTVL |
| | | RRCQEADREELNYWIRRYSD |
| | | AEDLKGGGGSDIQMTQSPSS |
| | | LSASVGDRVTITCRASQDIG |
| | | DRLRWYQQKPGKAPKLLIYH |
| | | GSRLESGVPSRFSGSRSGTD |
| | | FTLTISSLQPEDFATYYCQQ |
| | | QWERPYTEGQGTKVEIKR |

TABLE 6
DNA and amino acid sequences of NHR2 TDs

| SEQ | | |
|---|---|---|
| ID NO: | NHR2 TD | DNA SEQUENCE |
| 116 | NHR2 TD | CTAACAGACAGAGAATGGGCAGAAGAGTGGAAACATCTTGACCATCTGTTAAACTGCATA |
| | | ATGGACATGGTAGAAAAAACAAGGCGATCTCTCACCGTACTAAGGCGGTGTCAAGAAGCA |
| | | GACCGGGAAGAATTGAATTACTGGATCCGGCGGTACAGTGACGCCGAG |
| 117 | NHR2* TD | TTGCATGGCACACGTCAAGAAGAAATGATTGATCACAGACTAACAGACAGAGAATGGGCA |
| | | GAAGAGTGGAAACATCTTGACCATCTGTTAAACTGCATAATGGACATGGTAGAAAAAACA |
| | | AGGCGATCTCTCACCGTACTAAGGCGGTGTCAAGAAGCAGACCGGGAAGAATTGAATTAC |
| | | TGGATCCGGCGGTACAGTGACGCCGAG |
| 118 | NHR2** TD | GGCACACGTCAAGAAGAAATGATTGATCACAGACTAACAGACAGAGAATGGGCAGAAGAG |
| | | TGGAAACATCTTGACCATCTGTTAAACTGCATAATGGACATGGTAGAAAAAACAAGGCGA |
| | | TCTCTCACCGTACTAAGGCGGTGTCAAGAAGCAGACCGGGAAGAATTGAATTACTGGATC |
| | | CGGCGGTACAGTGACGCCGAG |
| | NHR2 TD | AMINO ACID SEQUENCE |
| 119 | NHR2*** TD | CAAGAAGAAATGATTGATCACAGACTAACAGACAGAGAATGGGCAGAAGAGTGGAAACAT |
| | | CTTGACCATCTGTTAAACTGCATAATGGACATGGTAGAAAAAACAAGGCGATCTCTCACC |
| | | GTACTAAGGCGGTGTCAAGAA |
| 120 | NHR2 TD | LTDREWAEEWKHLDHLLNCIMDMVEKTRRSLTVLRRCQEADREELNYWIRRYSDAE |
| 121 | NHR2* TD | LHGTRQEEMIDHRLTDREWAEEWKHLDHLLNCIMDMVEKTRRSLTVLRRCQEADREELNY |
| | | WIRRYSDAEDLK |
| 122 | NHR2** TD | GTRQEEMIDHRLTDREWAEEWKHLDHLLNCIMDMVEKTRRSLTVLRRCQEADREELNYWI |
| | | RRYSDAE |
| 123 | NHR2*** TD | QEEMIDHRLTDREWAEEWKHLDHLLNCIMDMVEKTRRSLTVLRRCQE |

NHR2* TD and NHR2** TD include additional amino acid residues at the N- and/or C-terminus. NHR2*** only includes amino acid residues of the annotated NHR2 domain according to Pubmed (Reference: UniProtKB-Q06455 (MTG8 HUMAN)

TABLE 7
Human p53 TD sequences

| SEQ | | |
|---|---|---|
| ID NO: | p53 TD | DNA SEQUENCE |
| 124 | p53 TD | AAGAAGAAACCACTGGATGGAGAATATTTCACCCTTCAGATCCGTGGGCGTGAGCGCTTCGAG |
| | | ATGTTCCGAGAGCTGAATGAGGCCTTGGAACTCAAGGATGCCCAGGCTGGGAAGGAGCCAGG |
| | | G |
| 125 | p53* TD | GGAGAATATTTCACCCTTCAGATCCGTGGGCGTGAGCGCTTCGAGATGTTCCGAGAGCTGAAT |
| | | GAGGCCTTGGAACTCAAGGATGCCCAGGCTGGG |
| SEQ | | |
| ID NO: | p53 TD | AMINO ACID SEQUENCE |
| 126 | p53 TD | KKKPLDGEYFTLQIRGRERFEMFRELNEALELKDAQAGKEPG |
| 10 | p53* TD | GEYFTLQIRGRERFEMFRELNEALELKDAQAG |

p53* TD is a truncated version of p53 TD

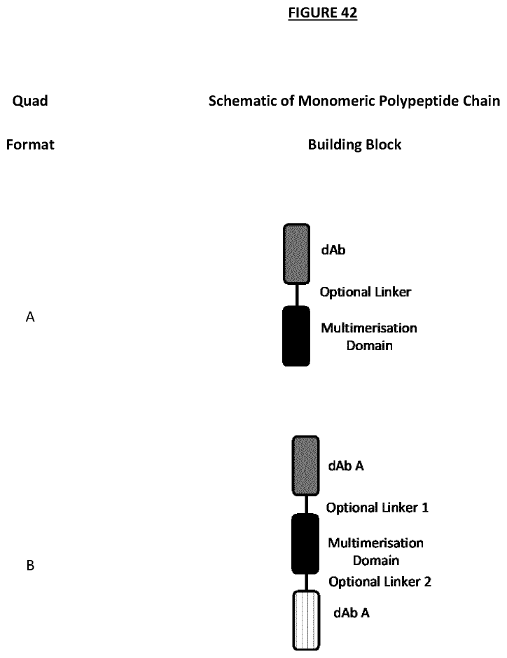

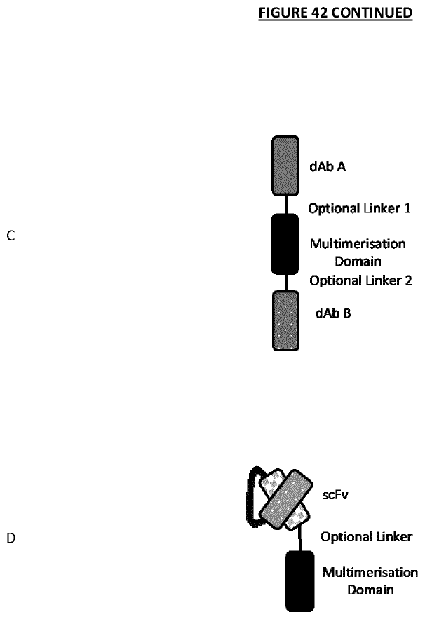

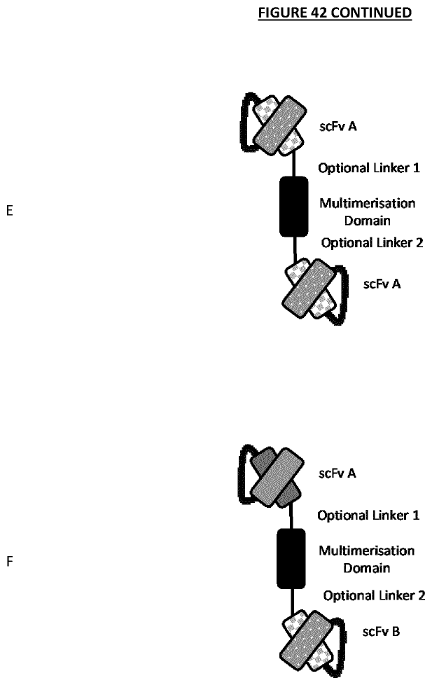

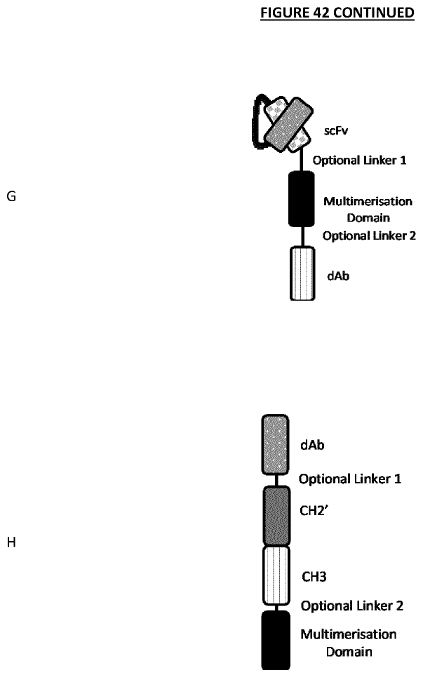

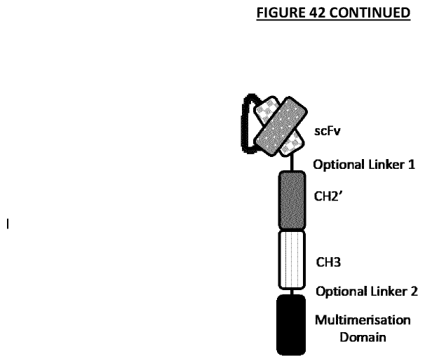

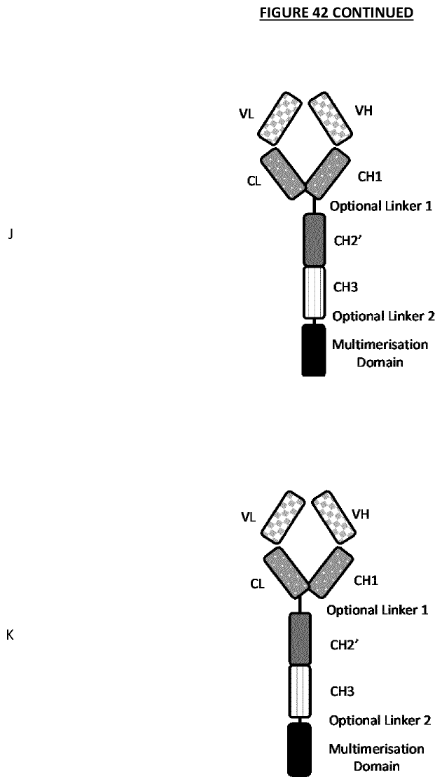

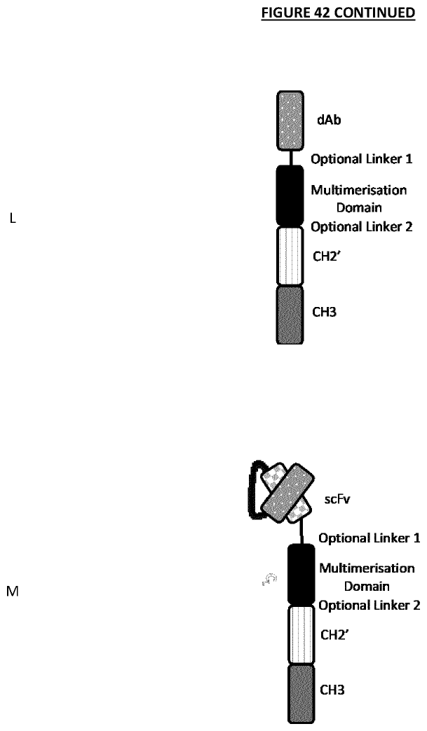

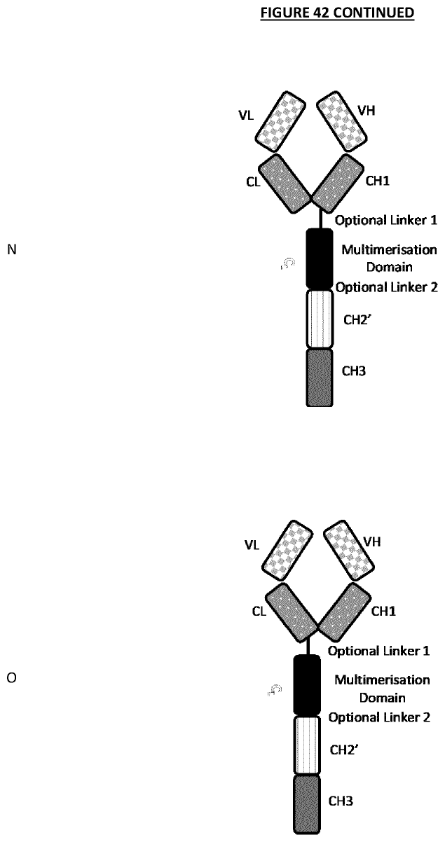

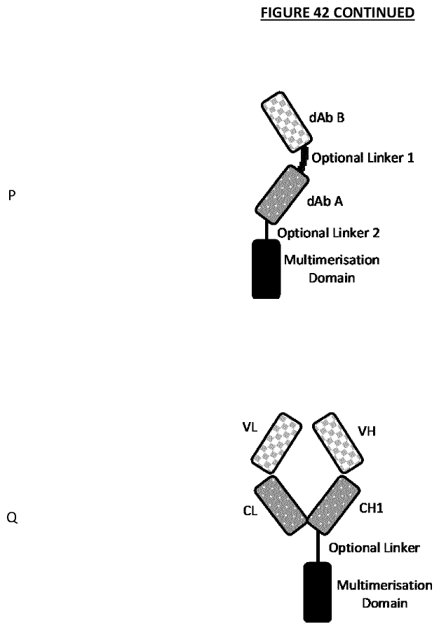

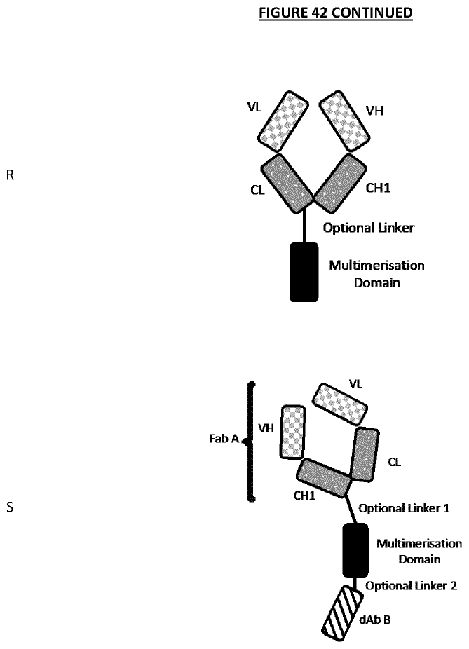

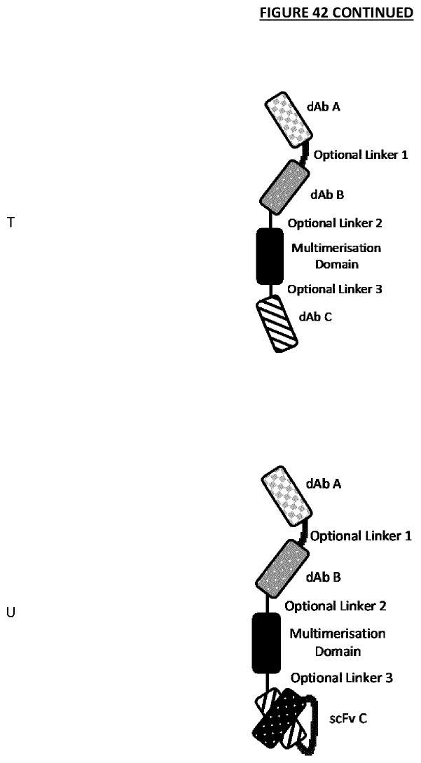

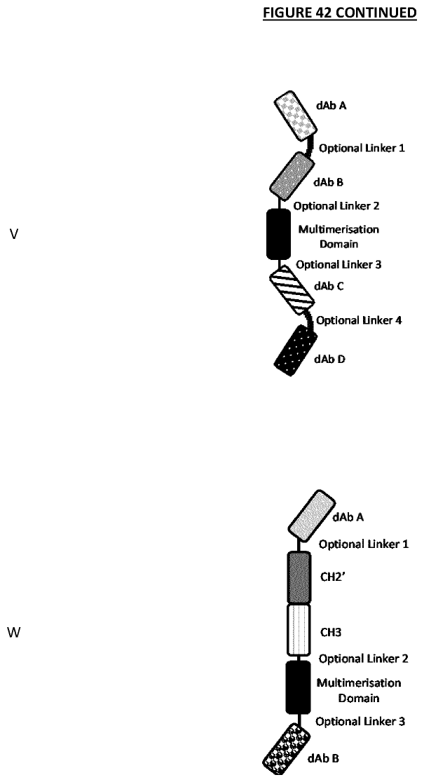

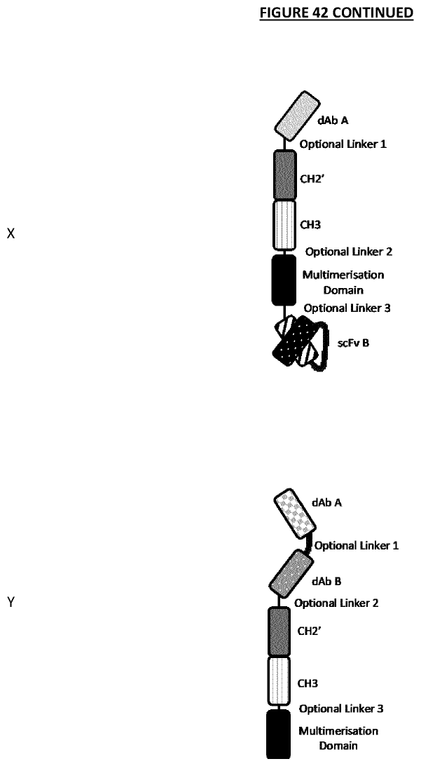

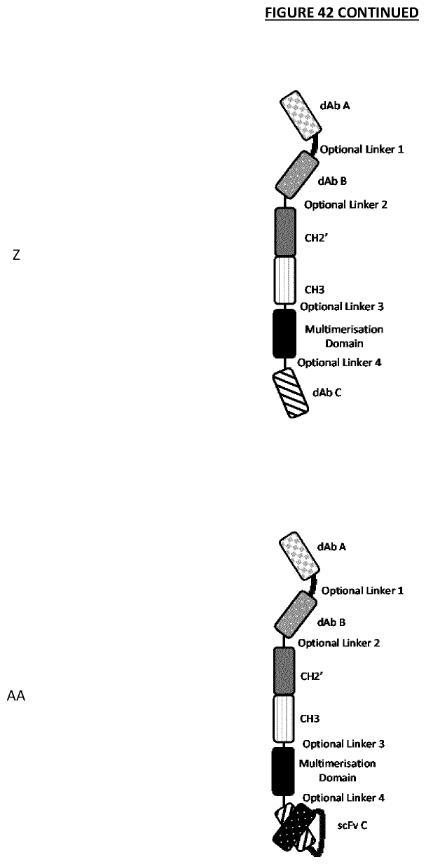

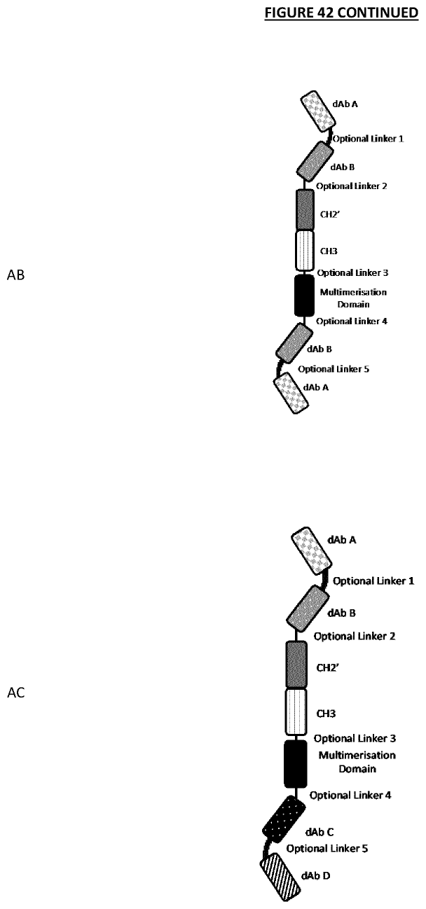

TABLE 8
Description of monomeric building block of Quad Formats
A-AC and as outlined in FIGS. 22 and 42.
In the schematics, components of polypeptide chains are
N- to C-terminally from top to the bottom of each chain
as shown.
Herein the terms “SAM”, “self-associating multimerization
domain” and “multimerisation domain” are used
interchangeably. A SAM may, for example be called a
TD herein. A TD may be a tetradimerisation domain as
disclosed herein, eg, a p53, p63, p73 or NHR2 domain
or homologue or orthologue thereof.
Valency indicates binding site number in the tetramer form
when using a TD as a SAM.

| Quad | |
|---|---|
| Format | Description |
| A | Tetravalent dAb Quad |
| | Monomeric building block of Quad format ‘A’ contains |
| | self-assembling multimerisation (SAM) domain linked to a |
| | dAb (an antibody single variable domain). The dAb can be |
| | linked at either the N- or C-terminus of the SAM domain |
| | via an optional peptide linker. The dAb binding domain |
| | can either be a VH, Vκ or Vλ. |
| B | Octavalent Monospecific dAb Quad |
| | Monomeric building block of Quad format ‘B’ contains |
| | SAM domain linked to dAb binding domains at both N- |
| | and C-terminus via optional peptide linkers 1 and/or 2. |
| | dAb binding domains can either be a VH, Vκ or Vλ. The |
| | dAbs have the same antigen specificity. |
| C | Octavalent Bispecific dAb Quad |
| | Monomeric building block of Quad format ‘C’ contains |
| | SAM domain linked to two different dAb binding domains |
| | (dAb A and dAb B) at the N- and C-terminus via optional |
| | peptide linkers 1 and/or 2. The dAb binding domains can |
| | either be a VH, Vκ or Vλ. dAb A has an antigen specificity |
| | that is different from the antigen specificity of dAbB. |
| D | Tetravalent Monospecific scFv Quad |
| | Monomeric building block of Quad format ‘D’ |
| | contains SAM domain linked to an scFv binding |
| | domain. The scFv binding domain can be linked |
| | at either the N- or C-terminus of the SAM |
| | domain via an optional peptide linker. |
| E | Octavalent Monospecific scFv Quad |
| | Monomeric building block of Quad format ‘E’ |
| | contains SAM domain linked to the same type |
| | of scFv binding domains at both N- and C- |
| | terminus via optional peptide linkers 1 and/or |
| | 2. |
| F | Octavalent Bispecific scFv Quad |
| | Monomeric building block of Quad format ‘F’ |
| | contains SAM domain linked to two different |
| | scFv binding domains (scFv A and scFv B) at the |
| | N- and C-terminus via optional peptide linkers 1 |
| | and/or 2. scFv A has an antigen specificity that |
| | is different from the antigen specificity of scFv |
| | B. |
| G | Octavalent Bispecific scFv × dAb Quad |
| | Monomeric building block of Quad format ‘G’ |
| | contains SAM domain linked to two different |
| | binding domain formats (i.e. scFv and a dAb). |
| | The scFv and dAb binding domains can be fused |
| | to SAM domain via the N- and C-terminus, |
| | respectively or via C- and N-terminus, |
| | respectively. Both scFv and dAb binding |
| | domains are fused to SAM domain via optional |
| | peptide linkers 1 and/or 2. |
| H | Tetravalent Monospecific Ig-dAb Quad v1 |
| | Monomeric building block of Quad format ‘H’ |
| | contains SAM domain linked to Ig Fc region (eg, |
| | an IgG1 Fc region) comprising of CH3 and CH2 |
| | (eg, CH2′) domains in addition to a dAb. dAb |
| | binding domain can be linked to N- or C- |
| | terminus of the Fc region via an optional |
| | peptide linker 1 or 2, respectively. Similarly |
| | SAM domain can be linked to N- or C-terminus |
| | of the Fc region via an optional peptide linker 1 |
| | or 2, respectively. CH2′ domain is devoid of |
| | core hinge region. The dAb binding domain can |
| | either be a VH, Vκ or Vλ. “Monomeric Ig” |
| | indicates that the Fc (such as comprising a |
| | CH2′) does not directly pair with another Fc. |
| I | Tetravalent Monospecific Ig-scFv Quad v1 |
| | Monomeric building block of Quad format ‘I’ |
| | contains SAM domain linked to Ig Fc region |
| | consisting of CH3 and CH2 (eg, CH2′) domains |
| | in addition to a scFv binding domain. scFv |
| | binding domain can be linked to N- or C- |
| | terminus of the Fc region via an optional |
| | peptide linker 1 or 2, respectively. Similarly |
| | SAM domain can be linked to N- or C-terminus |
| | of the Fc region via an optional peptide linker 1 |
| | or 2, respectively. CH2′ domain is devoid of |
| | core hinge region or a CXXC core region motif. |
| J | Tetravalent Monospecific Ig-Fab Quad v1 |
| | Monomeric building block of Quad format ‘J’ |
| | comprises or consists of two chains. The chain |
| | containing SAM domain is linked to Ig Fc region |
| | comprising CH3 and CH2 (eg, CH2’) domains in |
| | addition to Ig Fab connected via the CH1 |
| | domain. Ig Fab is linked to the N-terminus of |
| | the Fc region via an optional peptide linker 1. |
| | The SAM domain is linked to C-terminus of the |
| | Fc region via an optional peptide linker 2. CH2′ |
| | domain is devoid of core hinge region. The |
| | second chain is an Ig light chain comprising of |
| | CL and VL domains. |
| K | Tetravalent Monospecific Ig-Fab Quad v2 |
| | Monomeric building block of Quad format ‘K’ |
| | comprises or consists of two chains. The chain |
| | containing SAM domain is linked to Ig Fc region |
| | comprising CH3 and CH2 (eg, CH2′) domains in |
| | addition to Ig Fab connected via the CL domain. |
| | Ig Fab is linked to the N-terminus of the Fc |
| | region via an optional peptide linker 1. The |
| | SAM domain is linked to C-terminus of the Fc |
| | region via an optional peptide linker 2. CH2′ |
| | domain is devoid of core hinge region. The |
| | second chain is an Ig heavy chain comprising |
| | CH1 and VH domains. |
| L | Tetravalent Monospecific Ig-dAb Quad v2 |
| | Monomeric building block of Quad format ‘L’ |
| | contains SAM domain linked to Ig Fc region |
| | consisting of CH3 and CH2 (eg, CH2′) domains |
| | in addition to a dAb antigen binding domain. |
| | dAb is linked to the SAM domain with an |
| | optional peptide linker 1. The Fc region is |
| | linked to the SAM domain via the C-terminus |
| | with an optional linker 2. CH2′ domain is devoid |
| | of core hinge region. The dAb can either be a |
| | VH, Vκ or Vλ. |
| M | Tetravalent Monospecific Ig-scFv Quad v2 |
| | Monomeric building block of Quad format ‘M’ |
| | contains SAM domain linked to Ig Fc region |
| | consisting of CH3 and CH2 (eg, CH2′) domains |
| | in addition to a scFv antigen binding domain. |
| | scFv is linked to the SAM domain via the N- |
| | terminus with an optional peptide linker 1. The |
| | Fc region is linked to the SAM domain via the C- |
| | terminus with an optional linker 2. CH2′ |
| | domain is devoid of core hinge region. |
| N | Tetravalent Monospecific Ig-Fab Quad v3 |
| | Monomeric building block of Quad format ‘N’ |
| | comprises or consists of two chains. The chain |
| | containing SAM domain is linked to Ig Fc |
| | region comprising CH3 and CH2 (eg, CH2′) |
| | domains in addition to Ig Fab connected via |
| | the CH1 domain. The Ig Fab is linked to SAM |
| | domain via the N-terminus with an optional |
| | peptide linker 1. The Ig Fc region is linked to |
| | SAM domain via the C-terminus with an |
| | optional peptide linker 2. The second chain is |
| | an Ig light chain consisting of CL and VL |
| | domains. |
| O | Tetravalent Monospecific Ig-Fab Quad v4 |
| | Monomeric building block of Quad format ‘O’ |
| | comprises or consists of two chains. The chain |
| | containing SAM domain is linked to Ig Fc |
| | region consisting of CH3 and CH2 (eg, CH2′) |
| | domains in addition to Ig Fab connected via |
| | the CL domain. The Ig Fab is linked to SAM |
| | domain via the N-terminus with an optional |
| | peptide linker 1. The Ig Fc region is linked to |
| | SAM domain via the C-terminus with an |
| | optional peptide linker 2. The second chain is |
| | an Ig heavy chain consisting of CH1 and VH |
| | domains. |
| P | Octavalent Bispecific Tandem dAb Quad |
| | Monomeric building block of Quad format ‘P’ |
| | contains SAM domain linked to two different |
| | dAbs (dAb A and dAb B) connected in tandem. |
| | The dAbs are connected together via an |
| | optional peptide linker 1 and the tandem dAbs |
| | are connected to SAM domain at either the N- |
| | or C-terminus via an optional peptide linker 2. |
| | dAb binding domains can either be a VH, Vκ or |
| | Vλ. |
| Q | Tetravalent Fab Quad v1 |
| | Monomeric building block of Quad format ‘Q’ |
| | comprises or consists of two chains. The chain |
| | containing SAM domain is linked to Ig Fab via |
| | CH1 domain. Ig Fab is linked to the N-terminus |
| | of the SAM domain via an optional peptide |
| | linker. The second chain is an Ig light chain |
| | comprising CL and VL domains. |
| R | Tetravalent Fab Quad v2 |
| | Monomeric building block of Quad format ‘R’ |
| | comprises or consists of two chains. The chain |
| | containing SAM domain is linked to Ig Fab via |
| | CL domain. Ig Fab is linked to the N-terminus |
| | of the SAM domain via an optional peptide |
| | linker. The second chain is an Ig heavy chain |
| | comprising of CH1 and VH domains. |
| S | Octavalent Bispecific Fab × dAb Quad |
| | Monomeric building block of Quad format ‘S’ |
| | comprises or consists of two chains. The chain |
| | containing SAM domain is linked to different |
| | binding domains. Binding domain ‘A’ is a Fab |
| | linked to SAM domain at the N-terminus via |
| | an optional peptide linker 1. Binding domain |
| | ‘B’ is a dAb linked to the SAM domain at the C- |
| | terminus via an optional linker 2. The dAb |
| | binding domain can either be a VH, Vκ or Vλ. |
| | The second chain is an Ig light chain |
| | comprising CL and VL domains. The Fab |
| | binding domain ‘A’ could also be optionally |
| | linked via the CL of the light chain. |
| T | 12-Valent Trispecific dAb Quad |
| | Monomeric building block of Quad format ‘T’ |
| | contains SAM domain linked to three different |
| | dAbs (dAbs A, B & C) with specificity for either |
| | three different epitopes of the same target |
| | protein or a respective epitope on three |
| | different target proteins. The tandem dAbs ‘A’ |
| | & ‘B’ is linked to the SAM domain at the N- |
| | terminus via an optional linker 1 where dAbs |
| | ‘A’ & ‘B’ are linked together via an optional |
| | linker 2. A single dAb (dAb ‘C’) is linked at the |
| | C-terminus of the SAM domain via an optional |
| | linker 3. The dAb binding domains can either |
| | be a VH, Vκ or Vλ. In an alternative, the |
| | configuration (in N- to C-terminal direction) is |
| | dAb A - optional linker 1 - SAM optional linker |
| | 2 - dAb B - optional linker 3 - dAb C. |
| U | 12-Valent Trispecific dAb × scFv Quad |
| | Monomeric building block of Quad format ‘U’ |
| | contains SAM domain linked to three different |
| | binding domains (Tandem dAb containing |
| | dAbs A & B and an scFy ‘C’) with specificity for |
| | either three different epitopes of the same |
| | target protein or a respective epitope on three |
| | different target proteins. The tandem dAbs ‘A’ |
| | & ‘B’ is linked to the SAM domain at the N- |
| | terminus via an optional linker 1 where dAbs |
| | ‘A’ & ‘B’ are linked together via an optional |
| | linker 2. An scFv ‘C’ is linked at the C-terminus |
| | of the SAM domain via an optional linker 3. |
| | The dAb binding domains can either be a VH, |
| | Vκ or Vλ. In an alternative, the configuration |
| | (in N- to C-terminal direction) is (i) dAb A - |
| | optional linker 1 - SAM optional linker 2 - dAb |
| | B - optional linker 3 - scFv C; (ii) scFv- optional |
| | linker 1 - SAM optional linker 2 - dAb A - |
| | optional linker 3 - dAb B; or (iii) scFv- optional |
| | linker 1 - dAb A - optional linker 2 - SAM - |
| | optional linker 3 - dAb B. |
| V | 16-Valent Tetraspecific dAb Quad |
| | Monomeric building block of Quad format ‘V’ |
| | contains SAM domain linked to two tandem |
| | dAbs (consisting of dAbs A-D) with specificity |
| | for either four different epitopes of the same |
| | target protein or a respective epitope on four |
| | different target proteins. Each tandem dAb |
| | comprises two different dAbs linked together |
| | via one or more optional peptide linkers |
| | (Linker 1 and 4). The two tandem dAbs are |
| | linked to the SAM domain via the N- and C- |
| | terminus via optional peptide linkers 2 and 3 |
| | respectively. The dAb binding domains can |
| | either be a VH, Vκ or Vλ. In an alternative, all |
| | dAbs are the same and have the same epitope |
| | specificity. |
| W | Octavalent Bispecific Monomeric Ig-dAb |
| | Quad |
| | Monomeric building block of Quad format ‘W’ |
| | contains SAM domain linked to Ig Fc region |
| | comprising or consisting of CH3 and (CH2 (eg, |
| | CH2′) domains plus two different dAb binding |
| | domains. The Ig Fc region is linked to the SAM |
| | domain at the N-terminus via an optional |
| | peptide linker 2. The first dAb binding domain |
| | ‘A’ is linked to the Ig Fc region at the N- |
| | terminus via an optional peptide linker 1. The |
| | second dAb binding domain ‘B’ is linked to the |
| | SAM domain at the C-terminus via an optional |
| | peptide linker 3. The CH2′ domain is devoid of |
| | core hinge region. The dAb binding domains |
| | can either be a VH, Vκ or Vλ. In an |
| | alternative, the dAbs have the same antigen |
| | binding specificity. |
| X | Octavalent Bispecific Monomeric Ig-dAb × |
| | scFv Quad |
| | Monomeric building block of Quad format ‘X’ |
| | contains SAM domain linked to Ig Fc region |
| | comprising or consisting ofcCH3 and CH2 (eg, |
| | CH2′) domains plus two different binding |
| | domains. The Ig Fc region is linked to the SAM |
| | domain at the N-terminus via an optional |
| | peptide linker 2. The first binding domain (dAb |
| | A′) is linked to the Ig Fc region at the N- |
| | terminus via an optional peptide linker 1. The |
| | second binding domain (scFy ‘B’) is linked to |
| | the SAM domain at the C-terminus via an |
| | optional peptide linker 3. The CH2′ domain is |
| | devoid of core hinge region. The dAb binding |
| | domain can either be a VH, Vκ or Vλ. In an |
| | alternative, the dAb and scFy have the same |
| | antigen binding specificity. |
| Y | Octavalent Bispecific Monomeric Ig-Tandem |
| | dAb Quad |
| | Monomeric building block of Quad format ‘Y’ |
| | contains SAM domain linked to Ig Fc region |
| | comprising or consisting of CH3 and CH2 (eg, |
| | CH2′) domains plus a tandem dAb containing |
| | dAbs ‘A’ & ‘B’ linked together via an optional |
| | peptide linker 1. The Ig Fc region is linked to |
| | the SAM domain at the N-terminus via an |
| | optional peptide linker 3. The tandem dAb is |
| | linked to the Ig Fc region at the N-terminus via |
| | an optional peptide linker 2. The CH2′ domain |
| | is devoid of core hinge region. The dAb |
| | binding domains can either be a VH, Vκ or Vλ. |
| | In an alternative, the dAbs have the same |
| | antigen binding specificity. |
| Z | 12-Valent Trispecific Monomeric Ig-dAb Quad |
| | Monomeric building block of Quad format ‘Z’ |
| | contains SAM domain linked to Ig Fc region |
| | comprising or consisting of CH3 and CH2 (eg, |
| | CH2′) domains plus a tandem dAb containing |
| | dAbs ‘A’ & ‘B’ linked together via an optional |
| | peptide linker 1 and a single dAb binding |
| | domain ‘C’. The Ig Fc region is linked to the |
| | SAM domain at the N-terminus via an optional |
| | peptide linker 3. The tandem dAb is linked to |
| | the Ig Fc region at the N-terminus via an |
| | optional peptide linker 2. The dAb binding |
| | domain ‘C’ is linked to the SAM domain at the |
| | C-terminus via an optional peptide linker 4. |
| | The CH2′ domain is devoid of core hinge |
| | region. The dAb binding domains can either be |
| | a VH, Vκ or Vλ. In an alternative, the dAbs |
| | have the same antigen binding specificity. |
| AA | 12-Valent Trispecific Monomeric Ig-dAb × |
| | scFv Quad |
| | Monomeric building block of Quad format |
| | ‘AA’ contains SAM domain linked to Ig Fc |
| | region comprising or consisting of CH3 and |
| | CH2′ domains plus a tandem dAb containing |
| | dAbs ‘A’ & ‘B’ linked together via an optional |
| | peptide linker 1 and a scFy containing binding |
| | domain ‘C’. The Ig Fc region is linked to the |
| | SAM domain at the N-terminus via an optional |
| | peptide linker 3. The tandem dAb is linked to |
| | the Ig Fc region at the N-terminus via an |
| | optional peptide linker 2. The scFy binding |
| | domain ‘C’ is linked to the SAM domain at the |
| | C-terminus via an optional peptide linker 4. |
| | The CH2′ domain is devoid of core hinge |
| | region. The dAb binding domains can either be |
| | a VH, Vκ or Vλ. In an alternative, the dAbs |
| | and scFy have the same antigen binding |
| | specificity. |
| AB | 16-Valent Bispecific Monomeric Ig-Tandem |
| | dAb Quad |
| | Monomeric building block of Quad format ‘AB’ |
| | contains SAM domain linked to Ig Fc region |
| | comprising or consisting of CH3 and CH2 (eg, |
| | CH2′) domains plus two identical tandem dAbs |
| | containing dAbs ‘A’ & ‘B’ linked together via |
| | optional peptide linkers 1 and 5, respectively. |
| | The Ig Fc region is linked to the SAM domain |
| | at the N-terminus via an optional peptide |
| | linker 3. The first tandem dAb is linked to the |
| | Ig Fc region at the N-terminus via an optional |
| | peptide linker 2. The second tandem dAb is |
| | linked to the SAM domain at the C-terminus |
| | via an optional peptide linker 4. The CH2′ |
| | domain is devoid of core hinge region. The |
| | dAb binding domains can either be a VH, Vκ |
| | or Vλ. In an alternative, the dAbs have the |
| | same antigen binding specificity. |
| AC | 16-Valent Tetraspecific Monomeric Ig- |
| | Tandem dAb Quad |
| | Monomeric building block of Quad format ‘AC’ |
| | contains SAM domain linked to Ig Fc region |
| | comprising or consisting of CH3 and CH2 (eg, |
| | CH2′) domains plus two different tandem |
| | dAbs containing dAbs ‘A’ & ‘B’ and dAbs ‘C’ & |
| | ‘D’ linked together via optional peptide linkers |
| | 1 and 5 respectively. The Ig Fc region is linked |
| | to the SAM domain at the N-terminus via an |
| | optional peptide linker 3. The first tandem |
| | dAb containing dAbs ‘A’ & ‘B’ is linked to the |
| | Ig Fc region at the N-terminus via an optional |
| | peptide linker 2. The second tandem dAb |
| | containing dAbs ‘C’ & ‘D’ is linked to the SAM |
| | domain at the C-terminus via an optional |
| | peptide linker 4. The CH2′ domain is devoid of |
| | core hinge region. The dAb binding domains |
| | can either be a VH, Vκ or Vλ. In an |
| | alternative, the dAbs AB or CD or AC or AD or |
| | BC or BD have the same antigen binding |
| | specificity. For example AD have a first |
| | antigen specificity and BC have a second |
| | antigen specificity. For example AB have a |
| | first antigen specificity and CD have a second |
| | antigen specificity |

TABLE 9
DNA sequences encoding Quad polypeptides

| SEQ ID NO. | QUAD NO. | NUCLEOTIDE SEQUENCE |
|---|---|---|
| 139 | Quad 51 | ATGGGATGGTCTTGTATAATTCTGTTCCTGGTGGCAACAGCAA |
| | | CAGGAGTGCATAGCGAGGTGCAGCTGGTGGAGTCTGGGGGA |
| | | GGCTTGGTACAGCCCGGCAGGTCCCTGAGACTCTCCTGTGCGG |
| | | CCTCTGGATTCACCTTTGATGATTATGCCATGCACTGGGTCCGG |
| | | CAAGCTCCAGGGAAGGGCCTGGAATGGGTCTCAGCTATCACTT |
| | | GGAATAGTGGTCACATAGACTATGCGGACTCTGTGGAGGGCC |
| | | GATTCACCATCTCCAGAGACAACGCCAAGAACTCCCTGTATCTG |
| | | CAAATGAACAGTCTGAGAGCTGAGGATACGGCCGTATATTACT |
| | | GTGCGAAAGTCTCGTACCTTAGCACCGCGTCCTCCCTTGACTAT |
| | | TGGGGCCAAGGTACCCTGGTCACCGTCTCGAGTGGCGGCGGA |
| | | GGCAGCGGAGGCGGAGGTTCTGGAGGAGGGGGGAGTGACAT |
| | | CCAGATGACCCAGTCTCCATCCTCCCTGTCTGCATCTGTAGGGG |
| | | ACAGAGTCACCATCACTTGTCGGGCAAGTCAGGGCATCAGAAA |
| | | TTACTTAGCCTGGTATCAGCAAAAACCAGGGAAAGCCCCTAAG |
| | | CTCCTGATCTATGCTGCATCCACTTTGCAATCAGGGGTCCCATC |
| | | TCGGTTCAGTGGCAGTGGATCTGGGACAGATTTCACTCTCACC |
| | | ATCAGCAGCCTACAGCCTGAAGATGTTGCAACTTATTACTGTCA |
| | | AAGGTATAACCGTGCACCGTATACTTTTGGCCAGGGGACCAAG |
| | | GTGGAAATCAAAAAGAAGAAACCACTGGATGGAGAATATTTC |
| | | ACCCTTCAGATCCGTGGGCGTGAGCGCTTCGAGATGTTCCGAG |
| | | AGCTGAATGAGGCCTTGGAACTCAAGGATGCCCAGGCTGGGA |
| | | AGGAGCCAGGG |
| 140 | Quad 53 Mon | ATGGGCTGGTCCTGCATCATCCTGTTTCTGGTGGCCACAGCCA |
| | | CAGGCGTGCACAGCGATATTGTGCTGACACAGAGCCCCGCCAT |
| | | CCTGAGTGCTTCTCCAGGCGAGAAAGTGACCATGACCTGCAGA |
| | | GCCAGCAGCAGCGTGAACTACATGGACTGGTATCAGAAGAAG |
| | | CCCGGCAGCAGCCCCAAGCCTTGGATCTACGCCACAAGCAATC |
| | | TGGCCAGCGGAGTGCCTGCCAGATTTTCTGGCTCTGGCAGCGG |
| | | CACAAGCTACAGCCTGACAATCAGCAGAGTGGAAGCCGAGGA |
| | | TGCCGCCACCTACTACTGTCAGCAGTGGTCCTTCAATCCTCCTA |
| | | CCTTCGGCGGAGGCACCAAGCTGGAAATCAAGGGCTCTACATC |
| | | TGGCGGAGGTGGAAGCGGAGGCGGAGGATCTGGTGGTGGTG |
| | | GATCTTCTGAGGTCCAGCTGCAACAGTCTGGCGCCGAGCTTGT |
| | | GAAACCTGGCGCCTCTGTGAAGATGAGCTGCAAGGCCAGCGG |
| | | CTACACCTTCACCAGCTACAACATGCACTGGGTCAAGCAGACC |
| | | CCTGGACAGGGACTCGAGTGGATCGGAGCCATCTATCCCGGC |
| | | AATGGCGACACCTCCTACAACCAGAAGTTCAAGGGCAAAGCCA |
| | | CACTGACCGCCGACAAGAGCAGCAGCACAGCCTACATGCAGCT |
| | | GAGCAGCCTGACCAGCGAGGACAGCGCCGATTACTACTGCGC |
| | | CAGAAGCAACTACTACGGCAGCTCCTACTGGTTCTTCGACGTG |
| | | TGGGGAGCCGGCACCACAGTGACAGTGTCCAGC |
| 141 | Quad 53 Tet | ATGGGCTGGTCCTGCATCATCCTGTTTCTGGTGGCCACAGCCA |
| | | CAGGCGTGCACAGCGATATTGTGCTGACACAGAGCCCCGCCAT |
| | | CCTGAGTGCTTCTCCAGGCGAGAAAGTGACCATGACCTGCAGA |
| | | GCCAGCAGCAGCGTGAACTACATGGACTGGTATCAGAAGAAG |
| | | CCCGGCAGCAGCCCCAAGCCTTGGATCTACGCCACAAGCAATC |
| | | TGGCCAGCGGAGTGCCTGCCAGATTTTCTGGCTCTGGCAGCGG |
| | | CACAAGCTACAGCCTGACAATCAGCAGAGTGGAAGCCGAGGA |
| | | TGCCGCCACCTACTACTGTCAGCAGTGGTCCTTCAATCCTCCTA |
| | | CCTTCGGCGGAGGCACCAAGCTGGAAATCAAGGGCTCTACATC |
| | | TGGCGGAGGTGGAAGCGGAGGCGGAGGATCTGGTGGTGGTG |
| | | GATCTTCTGAGGTCCAGCTGCAACAGTCTGGCGCCGAGCTTGT |
| | | GAAACCTGGCGCCTCTGTGAAGATGAGCTGCAAGGCCAGCGG |
| | | CTACACCTTCACCAGCTACAACATGCACTGGGTCAAGCAGACC |
| | | CCTGGACAGGGACTCGAGTGGATCGGAGCCATCTATCCCGGC |
| | | AATGGCGACACCTCCTACAACCAGAAGTTCAAGGGCAAAGCCA |
| | | CACTGACCGCCGACAAGAGCAGCAGCACAGCCTACATGCAGCT |
| | | GAGCAGCCTGACCAGCGAGGACAGCGCCGATTACTACTGCGC |
| | | CAGAAGCAACTACTACGGCAGCTCCTACTGGTTCTTCGACGTG |
| | | TGGGGAGCCGGCACCACAGTGACAGTGTCCAGCAAGAAAAAG |
| | | CCCCTGGACGGCGAGTACTTCACACTGCAGATCCGGGGCAGA |
| | | GAACGCTTCGAGATGTTCAGAGAGCTGAACGAGGCCCTGGAA |
| | | CTGAAGGATGCCCAGGCCGGAAAAGAGCCCGGC |
| 142 | Quad 53 Oct | ATGGGCTGGTCCTGCATCATCCTGTTTCTGGTGGCCACAGCCA |
| | | CAGGCGTGCACAGCGATATTGTGCTGACACAGAGCCCCGCCAT |
| | | CCTGAGTGCTTCTCCAGGCGAGAAAGTGACCATGACCTGCAGA |
| | | GCCAGCAGCAGCGTGAACTACATGGACTGGTATCAGAAGAAG |
| | | CCCGGCAGCAGCCCCAAGCCTTGGATCTACGCCACAAGCAATC |
| | | TGGCCAGCGGAGTGCCTGCCAGATTTTCTGGCTCTGGCAGCGG |
| | | CACAAGCTACAGCCTGACAATCAGCAGAGTGGAAGCCGAGGA |
| | | TGCCGCCACCTACTACTGTCAGCAGTGGTCCTTCAATCCTCCTA |
| | | CCTTCGGCGGAGGCACCAAGCTGGAAATCAAGGGCTCTACATC |
| | | TGGCGGAGGTGGAAGCGGAGGCGGAGGATCTGGTGGTGGTG |
| | | GATCTTCTGAGGTCCAGCTGCAACAGTCTGGCGCCGAGCTTGT |
| | | GAAACCTGGCGCCTCTGTGAAGATGAGCTGCAAGGCCAGCGG |
| | | CTACACCTTCACCAGCTACAACATGCACTGGGTCAAGCAGACC |
| | | CCTGGACAGGGACTCGAGTGGATCGGAGCCATCTATCCCGGC |
| | | AATGGCGACACCTCCTACAACCAGAAGTTCAAGGGCAAAGCCA |
| | | CACTGACCGCCGACAAGAGCAGCAGCACAGCCTACATGCAGCT |
| | | GAGCAGCCTGACCAGCGAGGACAGCGCCGATTACTACTGCGC |
| | | CAGAAGCAACTACTACGGCAGCTCCTACTGGTTCTTCGACGTG |
| | | TGGGGAGCCGGCACCACAGTGACAGTGTCCAGCAAGAAAAAG |
| | | CCCCTGGACGGCGAGTACTTCACACTGCAGATCCGGGGCAGA |
| | | GAACGCTTCGAGATGTTCAGAGAGCTGAACGAGGCCCTGGAA |
| | | CTGAAGGATGCCCAGGCCGGAAAAGAGCCCGGCGATATTGTG |
| | | CTGACACAGAGCCCCGCCATCCTGAGTGCTTCTCCAGGCGAGA |
| | | AAGTGACCATGACCTGCAGAGCCAGCAGCAGCGTGAACTACA |
| | | TGGACTGGTATCAGAAGAAGCCCGGCAGCAGCCCCAAGCCTT |
| | | GGATCTACGCCACAAGCAATCTGGCCAGCGGAGTGCCTGCCA |
| | | GATTTTCTGGCTCTGGCAGCGGCACAAGCTACAGCCTGACAAT |
| | | CAGCAGAGTGGAAGCCGAGGATGCCGCCACCTACTACTGTCA |
| | | GCAGTGGTCCTTCAATCCTCCTACCTTCGGCGGAGGCACCAAG |
| | | CTGGAAATCAAGGGCTCTACATCTGGCGGAGGTGGAAGCGGA |
| | | GGCGGAGGATCTGGTGGTGGTGGATCTTCTGAGGTCCAGCTG |
| | | CAACAGTCTGGCGCCGAGCTTGTGAAACCTGGCGCCTCTGTGA |
| | | AGATGAGCTGCAAGGCCAGCGGCTACACCTTCACCAGCTACAA |
| | | CATGCACTGGGTCAAGCAGACCCCTGGACAGGGACTCGAGTG |
| | | GATCGGAGCCATCTATCCCGGCAATGGCGACACCTCCTACAAC |
| | | CAGAAGTTCAAGGGCAAAGCCACACTGACCGCCGACAAGAGC |
| | | AGCAGCACAGCCTACATGCAGCTGAGCAGCCTGACCAGCGAG |
| | | GACAGCGCCGATTACTACTGCGCCAGAAGCAACTACTACGGCA |
| | | GCTCCTACTGGTTCTTCGACGTGTGGGGAGCCGGCACCACAGT |
| | | GACAGTGTCCAGC |
| 143 | Quad 54 | atgggatggtcttgtataattctgttcctggtggcaacagcaacaggagtgcatagc |
| | | GAGGTGCAGCTGGTTGAATCTGGCGGAGGACTGGTTCAGCCT |
| | | GGCGGATCTCTGAGACTGTCTTGTGCCGCCAGCGGCTTCACCT |
| | | TCAGCGACTACTGGATGTACTGGGTCCGACAGGCCCCTGGCAA |
| | | AGGCCTTGAATGGGTGTCCGAGATCAACACCAACGGCCTGATC |
| | | ACAAAGTACCCCGACAGCGTGAAGGGCAGATTCACCATCAGCC |
| | | GGGACAACGCCAAGAACACCCTGTACCTGCAGATGAACAGCCT |
| | | GCGGCCTGAGGATACCGCCGTGTACTACTGTGCCAGATCTCCC |
| | | AGCGGATTCAACAGAGGCCAGGGCACACTGGTCACCGTGTCA |
| | | TCTAAGAAGAAACCACTGGATGGAGAATATTTCACCCTTCAGA |
| | | TCCGTGGGCGTGAGCGCTTCGAGATGTTCCGAGAGCTGAATG |
| | | AGGCCTTGGAACTCAAGGATGCCCAGGCTGGGAAGGAGCCAG |
| | | GGGAGGTGCAGCTGGTTGAATCTGGCGGAGGACTGGTTCAGG |
| | | CTGGCGGATCTCTGAGACTGTCTTGTGCCGCCAGCGGCAGCAG |
| | | CTTCAGATTCAGAGCTATGGCCTGGTACAGACAGGCCCCTGAG |
| | | AAGCAGAGGGATTTCGTGGCCACCATCAACAGCCTGGGCGAG |
| | | ACAACATATGCCACCGCCGTGGAAGGCCGGTTCACCATCAGCA |
| | | GAGACAACGCCAAGAACACCGTGTACCTGCAGATGGACAGCC |
| | | TGAAGCCTGAGGATACCGCCGTGTACTACTGCAACGAGCCCAG |
| | | AGGCAATTACTGGGGCCAGGGCACACAAGTGACCGTGTCATCT |
| | | CAC |
| 144 | Quad 55 | atgggatggtcttgtataattctgttcctggtggcaacagcaacaggagtgcatagc |
| | | GAGGTGCAGCTGGTGGAGTCTGGGGGAGGCTTGGTACAGCCC |
| | | GGCAGGTCCCTGAGACTCTCCTGTGCGGCCTCTGGATTCACCTT |
| | | TGATGATTATGCCATGCACTGGGTCCGGCAAGCTCCAGGGAAG |
| | | GGCCTGGAATGGGTCTCAGCTATCACTTGGAATAGTGGTCACA |
| | | TAGACTATGCGGACTCTGTGGAGGGCCGATTCACCATCTCCAG |
| | | AGACAACGCCAAGAACTCCCTGTATCTGCAAATGAACAGTCTG |
| | | AGAGCTGAGGATACGGCCGTATATTACTGTGCGAAAGTCTCGT |
| | | ACCTTAGCACCGCGTCCTCCCTTGACTATTGGGGCCAAGGTACC |
| | | CTGGTCACCGTCTCGAGTGGCGGCGGAGGCAGCGGAGGCGG |
| | | AGGTTCTGGAGGAGGGGGGAGTGACATCCAGATGACCCAGTC |
| | | TCCATCCTCCCTGTCTGCATCTGTAGGGGACAGAGTCACCATCA |
| | | CTTGTCGGGCAAGTCAGGGCATCAGAAATTACTTAGCCTGGTA |
| | | TCAGCAAAAACCAGGGAAAGCCCCTAAGCTCCTGATCTATGCT |
| | | GCATCCACTTTGCAATCAGGGGTCCCATCTCGGTTCAGTGGCA |
| | | GTGGATCTGGGACAGATTTCACTCTCACCATCAGCAGCCTACA |
| | | GCCTGAAGATGTTGCAACTTATTACTGTCAAAGGTATAACCGT |
| | | GCACCGTATACTTTTGGCCAGGGGACCAAGGTGGAAATCAAA |
| | | AAGAAGAAACCACTGGATGGAGAATATTTCACCCTTCAGATCC |
| | | GTGGGCGTGAGCGCTTCGAGATGTTCCGAGAGCTGAATGAGG |
| | | CCTTGGAACTCAAGGATGCCCAGGCTGGGAAGGAGCCAGGGC |
| | | AGGTTCAGCTGGTTCAGTCTGGCGCCGAAGTGAAGAAACCTG |
| | | GCAGCAGCGTGAAGGTGTCCTGCAAGGCCAGCGGCTACAGCT |
| | | TCACCGACTACCACATCCACTGGGTCCGACAGGCTCCAGGACA |
| | | AGGCTTGGAATGGATGGGCGTGATCAACCCTATGTACGGCACC |
| | | ACCGATTACAACCAGCGGTTCAAGGGCAGAGTGACCATCACCG |
| | | CCGATGAGAGCACAAGCACCGCCTACATGGAACTGAGCAGCC |
| | | TGAGAAGCGAGGACACCGCCGTGTACTACTGCGCCAGATACG |
| | | ACTACTTTACCGGCACCGGGGTGTACTGGGGACAGGGAACAC |
| | | TGGTCACAGTGTCCTCTGGCGGCGGAGGCAGCGGAGGCGGAG |
| | | GTTCTGGAGGAGGGGGGAGTGACATCGTGATGACCCAGACAC |
| | | CTCTGAGCCTGAGCGTGACACCTGGACAGCCTGCCAGCATCAG |
| | | CTGCAGATCCAGCAGATCTCTGGTGCACAGCCGGGGCAATACC |
| | | TACCTGCACTGGTATCTGCAGAAGCCCGGCCAGTCTCCTCAGCT |
| | | GCTGATCTACAAGGTGTCCAACCGGTTCATCGGCGTGCCCGAT |
| | | AGATTTTCTGGCAGCGGCTCTGGCACCGACTTCACCCTGAAGA |
| | | TCTCCAGAGTGGAAGCCGAGGACGTGGGCGTGTACTACTGTA |
| | | GCCAGAGCACCCATCTGCCTTTCACCTTTGGCCAGGGCACCAA |
| | | GCTGGAAATCAAGCAC |
| 145 | Quad 56 | atgggatggtcttgtataattctgttcctggtggcaacagcaacaggagtgcatagc |
| | | GAGGTGCAGCTGGTGGAGTCTGGGGGAGGCTTGGTACAGCCC |
| | | GGCAGGTCCCTGAGACTCTCCTGTGCGGCCTCTGGATTCACCTT |
| | | TGATGATTATGCCATGCACTGGGTCCGGCAAGCTCCAGGGAAG |
| | | GGCCTGGAATGGGTCTCAGCTATCACTTGGAATAGTGGTCACA |
| | | TAGACTATGCGGACTCTGTGGAGGGCCGATTCACCATCTCCAG |
| | | AGACAACGCCAAGAACTCCCTGTATCTGCAAATGAACAGTCTG |
| | | AGAGCTGAGGATACGGCCGTATATTACTGTGCGAAAGTCTCGT |
| | | ACCTTAGCACCGCGTCCTCCCTTGACTATTGGGGCCAAGGTACC |
| | | CTGGTCACCGTCTCGAGTGGCGGCGGAGGCAGCGGAGGCGG |
| | | AGGTTCTGGAGGAGGGGGGAGTGACATCCAGATGACCCAGTC |
| | | TCCATCCTCCCTGTCTGCATCTGTAGGGGACAGAGTCACCATCA |
| | | CTTGTCGGGCAAGTCAGGGCATCAGAAATTACTTAGCCTGGTA |
| | | TCAGCAAAAACCAGGGAAAGCCCCTAAGCTCCTGATCTATGCT |
| | | GCATCCACTTTGCAATCAGGGGTCCCATCTCGGTTCAGTGGCA |
| | | GTGGATCTGGGACAGATTTCACTCTCACCATCAGCAGCCTACA |
| | | GCCTGAAGATGTTGCAACTTATTACTGTCAAAGGTATAACCGT |
| | | GCACCGTATACTTTTGGCCAGGGGACCAAGGTGGAAATCAAA |
| | | AAGAAGAAACCACTGGATGGAGAATATTTCACCCTTCAGATCC |
| | | GTGGGCGTGAGCGCTTCGAGATGTTCCGAGAGCTGAATGAGG |
| | | CCTTGGAACTCAAGGATGCCCAGGCTGGGAAGGAGCCAGGGG |
| | | AGGTGCAGCTGGTTGAATCTGGCGGAGGACTGGTTCAGGCTG |
| | | GCGGATCTCTGAGACTGTCTTGTGCCGCCAGCGGCAGCAGCTT |
| | | CAGATTCAGAGCTATGGCCTGGTACAGACAGGCCCCTGAGAA |
| | | GCAGAGGGATTTCGTGGCCACCATCAACAGCCTGGGCGAGAC |
| | | AACATATGCCACCGCCGTGGAAGGCCGGTTCACCATCAGCAGA |
| | | GACAACGCCAAGAACACCGTGTACCTGCAGATGGACAGCCTG |
| | | AAGCCTGAGGATACCGCCGTGTACTACTGCAACGAGCCCAGA |
| | | GGCAATTACTGGGGCCAGGGCACACAAGTGACCGTGTCATCTC |
| | | AC |
| 146 | Quad 57 | atgggatggtcttgtataattctgttcctggtggcaacagcaacaggagtgcatagc |
| | | GAGGTGCAGCTGGTTGAATCTGGCGGAGGACTGGTTCAGGCT |
| | | GGCGGATCTCTGAGACTGTCTTGTGCCGCCAGCGGCAGCAGCT |
| | | TCAGATTCAGAGCTATGGCCTGGTACAGACAGGCCCCTGAGAA |
| | | GCAGAGGGATTTCGTGGCCACCATCAACAGCCTGGGCGAGAC |
| | | AACATATGCCACCGCCGTGGAAGGCCGGTTCACCATCAGCAGA |
| | | GACAACGCCAAGAACACCGTGTACCTGCAGATGGACAGCCTG |
| | | AAGCCTGAGGATACCGCCGTGTACTACTGCAACGAGCCCAGA |
| | | GGCAATTACTGGGGCCAGGGCACACAAGTGACCGTGTCATCTA |
| | | AGAAGAAACCACTGGATGGAGAATATTTCACCCTTCAGATCCG |
| | | TGGGCGTGAGCGCTTCGAGATGTTCCGAGAGCTGAATGAGGC |
| | | CTTGGAACTCAAGGATGCCCAGGCTGGGAAGGAGCCAGGGCA |
| | | C |
| 147 | Quad 63 | atgggatggtcttgtataattctgttcctggtggcaacagcaacaggagtgcatagc |
| | | GAGGTGCAGCTGGTGGAGTCTGGGGGAGGCTTGGTACAGCCC |
| | | GGCAGGTCCCTGAGACTCTCCTGTGCGGCCTCTGGATTCACCTT |
| | | TGATGATTATGCCATGCACTGGGTCCGGCAAGCTCCAGGGAAG |
| | | GGCCTGGAATGGGTCTCAGCTATCACTTGGAATAGTGGTCACA |
| | | TAGACTATGCGGACTCTGTGGAGGGCCGATTCACCATCTCCAG |
| | | AGACAACGCCAAGAACTCCCTGTATCTGCAAATGAACAGTCTG |
| | | AGAGCTGAGGATACGGCCGTATATTACTGTGCGAAAGTCTCGT |
| | | ACCTTAGCACCGCGTCCTCCCTTGACTATTGGGGCCAAGGTACC |
| | | CTGGTCACCGTCTCGAGTGGCGGCGGAGGCAGCGGAGGCGG |
| | | AGGTTCTGGAGGAGGGGGGAGTGACATCCAGATGACCCAGTC |
| | | TCCATCCTCCCTGTCTGCATCTGTAGGGGACAGAGTCACCATCA |
| | | CTTGTCGGGCAAGTCAGGGCATCAGAAATTACTTAGCCTGGTA |
| | | TCAGCAAAAACCAGGGAAAGCCCCTAAGCTCCTGATCTATGCT |
| | | GCATCCACTTTGCAATCAGGGGTCCCATCTCGGTTCAGTGGCA |
| | | GTGGATCTGGGACAGATTTCACTCTCACCATCAGCAGCCTACA |
| | | GCCTGAAGATGTTGCAACTTATTACTGTCAAAGGTATAACCGT |
| | | GCACCGTATACTTTTGGCCAGGGGACCAAGGTGGAAATCAAA |
| | | GGTGGTGGTGGCTCCGGAGGCGGCGGCTCTGGTGGCGGTGG |
| | | CAGCAAGAAGAAACCACTGGATGGAGAATATTTCACCCTTCAG |
| | | ATCCGTGGGCGTGAGCGCTTCGAGATGTTCCGAGAGCTGAAT |
| | | GAGGCCTTGGAACTCAAGGATGCCCAGGCTGGGAAGGAGCCA |
| | | GGGCAC |
| 148 | Quad 64 | ATGGGCTGGTCCTGCATCATCCTGTTTCTGGTGGCCACAGCCA |
| | | CAGGCGTGCACAGCGATATTGTGCTGACACAGAGCCCCGCCAT |
| | | CCTGAGTGCTTCTCCAGGCGAGAAAGTGACCATGACCTGCAGA |
| | | GCCAGCAGCAGCGTGAACTACATGGACTGGTATCAGAAGAAG |
| | | CCCGGCAGCAGCCCCAAGCCTTGGATCTACGCCACAAGCAATC |
| | | TGGCCAGCGGAGTGCCTGCCAGATTTTCTGGCTCTGGCAGCGG |
| | | CACAAGCTACAGCCTGACAATCAGCAGAGTGGAAGCCGAGGA |
| | | TGCCGCCACCTACTACTGTCAGCAGTGGTCCTTCAATCCTCCTA |
| | | CCTTCGGCGGAGGCACCAAGCTGGAAATCAAGGGCTCTACATC |
| | | TGGCGGAGGTGGAAGCGGAGGCGGAGGATCTGGTGGTGGTG |
| | | GATCTTCTGAGGTCCAGCTGCAACAGTCTGGCGCCGAGCTTGT |
| | | GAAACCTGGCGCCTCTGTGAAGATGAGCTGCAAGGCCAGCGG |
| | | CTACACCTTCACCAGCTACAACATGCACTGGGTCAAGCAGACC |
| | | CCTGGACAGGGACTCGAGTGGATCGGAGCCATCTATCCCGGC |
| | | AATGGCGACACCTCCTACAACCAGAAGTTCAAGGGCAAAGCCA |
| | | CACTGACCGCCGACAAGAGCAGCAGCACAGCCTACATGCAGCT |
| | | GAGCAGCCTGACCAGCGAGGACAGCGCCGATTACTACTGCGC |
| | | CAGAAGCAACTACTACGGCAGCTCCTACTGGTTCTTCGACGTG |
| | | TGGGGAGCCGGCACCACAGTGACAGTGTCCAGCGGCGGAGGT |
| | | GGAAGCGGAGGCGGAGGATCTGGTGGTGGTGGATCTGCCCCT |
| | | GAACTGCTGGGCGGACCTTCCGTGTTCCTGTTCCCCCCAAAGCC |
| | | CAAGGACACCCTGATGATCTCCCGGACCCCCGAAGTGACCTGC |
| | | GTGGTGGTGGATGTGTCCCACGAGGACCCTGAAGTGAAGTTC |
| | | AATTGGTACGTGGACGGCGTGGAAGTGCACAACGCCAAGACC |
| | | AAGCCTAGAGAGGAACAGTACAACTCCACCTACCGGGTGGTG |
| | | TCCGTGCTGACCGTGCTGCACCAGGATTGGCTGAACGGCAAAG |
| | | AGTACAAGTGCAAGGTGTCCAACAAGGCCCTGCCTGCCCCCAT |
| | | CGAAAAGACCATCTCCAAGGCCAAGGGCCAGCCCCGGGAACC |
| | | CCAGGTGTACACACTGCCCCCTAGCAGGGACGAGCTGACCAA |
| | | GAACCAGGTGTCCCTGACCTGTCTCGTGAAAGGCTTCTACCCCT |
| | | CCGATATCGCCGTGGAATGGGAGTCCAACGGCCAGCCTGAGA |
| | | ACAACTACAAGACCACCCCCCCTGTGCTGGACTCCGACGGCTC |
| | | ATTCTTCCTGTACAGCAAGCTGACAGTGGACAAGTCCCGGTGG |
| | | CAGCAGGGCAACGTGTTCTCCTGCTCCGTGATGCACGAGGCCC |
| | | TGCACAACCACTACACCCAGAAGTCCCTGTCCCTGAGCCCCGG |
| | | CAAGAAGAAAAAGCCCCTGGACGGCGAGTACTTCACACTGCA |
| | | GATCCGGGGCAGAGAACGCTTCGAGATGTTCAGAGAGCTGAA |
| | | CGAGGCCCTGGAACTGAAGGATGCCCAGGCCGGAAAAGAGCC |
| | | CGGC |
| 149 | Quad 65 | ATGGGCTGGTCCTGCATCATCCTGTTTCTGGTGGCCACAGCCA |
| | | CAGGCGTGCACAGCGATATTGTGCTGACACAGAGCCCCGCCAT |
| | | CCTGAGTGCTTCTCCAGGCGAGAAAGTGACCATGACCTGCAGA |
| | | GCCAGCAGCAGCGTGAACTACATGGACTGGTATCAGAAGAAG |
| | | CCCGGCAGCAGCCCCAAGCCTTGGATCTACGCCACAAGCAATC |
| | | TGGCCAGCGGAGTGCCTGCCAGATTTTCTGGCTCTGGCAGCGG |
| | | CACAAGCTACAGCCTGACAATCAGCAGAGTGGAAGCCGAGGA |
| | | TGCCGCCACCTACTACTGTCAGCAGTGGTCCTTCAATCCTCCTA |
| | | CCTTCGGCGGAGGCACCAAGCTGGAAATCAAGGGCTCTACATC |
| | | TGGCGGAGGTGGAAGCGGAGGCGGAGGATCTGGTGGTGGTG |
| | | GATCTTCTGAGGTCCAGCTGCAACAGTCTGGCGCCGAGCTTGT |
| | | GAAACCTGGCGCCTCTGTGAAGATGAGCTGCAAGGCCAGCGG |
| | | CTACACCTTCACCAGCTACAACATGCACTGGGTCAAGCAGACC |
| | | CCTGGACAGGGACTCGAGTGGATCGGAGCCATCTATCCCGGC |
| | | AATGGCGACACCTCCTACAACCAGAAGTTCAAGGGCAAAGCCA |
| | | CACTGACCGCCGACAAGAGCAGCAGCACAGCCTACATGCAGCT |
| | | GAGCAGCCTGACCAGCGAGGACAGCGCCGATTACTACTGCGC |
| | | CAGAAGCAACTACTACGGCAGCTCCTACTGGTTCTTCGACGTG |
| | | TGGGGAGCCGGCACCACAGTGACAGTGTCCAGCAAGAAAAAG |
| | | CCCCTGGACGGCGAGTACTTCACACTGCAGATCCGGGGCAGA |
| | | GAACGCTTCGAGATGTTCAGAGAGCTGAACGAGGCCCTGGAA |
| | | CTGAAGGATGCCCAGGCCGGAAAAGAGCCCGGCGCCCCTGAA |
| | | CTGCTGGGCGGACCTTCCGTGTTCCTGTTCCCCCCAAAGCCCAA |
| | | GGACACCCTGATGATCTCCCGGACCCCCGAAGTGACCTGCGTG |
| | | GTGGTGGATGTGTCCCACGAGGACCCTGAAGTGAAGTTCAATT |
| | | GGTACGTGGACGGCGTGGAAGTGCACAACGCCAAGACCAAGC |
| | | CTAGAGAGGAACAGTACAACTCCACCTACCGGGTGGTGTCCGT |
| | | GCTGACCGTGCTGCACCAGGATTGGCTGAACGGCAAAGAGTA |
| | | CAAGTGCAAGGTGTCCAACAAGGCCCTGCCTGCCCCCATCGAA |
| | | AAGACCATCTCCAAGGCCAAGGGCCAGCCCCGGGAACCCCAG |
| | | GTGTACACACTGCCCCCTAGCAGGGACGAGCTGACCAAGAACC |
| | | AGGTGTCCCTGACCTGTCTCGTGAAAGGCTTCTACCCCTCCGAT |
| | | ATCGCCGTGGAATGGGAGTCCAACGGCCAGCCTGAGAACAAC |
| | | TACAAGACCACCCCCCCTGTGCTGGACTCCGACGGCTCATTCTT |
| | | CCTGTACAGCAAGCTGACAGTGGACAAGTCCCGGTGGCAGCA |
| | | GGGCAACGTGTTCTCCTGCTCCGTGATGCACGAGGCCCTGCAC |
| | | AACCACTACACCCAGAAGTCCCTGTCCCTGAGCCCCGGCAAG |
| 150 | W51ScFv | atgggatggtcttgtataattctgttcctggtggcaacagcaacaggagtgcatagc |
| | | GAGGTGCAGCTGGTGGAGTCTGGGGGAGGCTTGGTACAGCCC |
| | | GGCAGGTCCCTGAGACTCTCCTGTGCGGCCTCTGGATTCACCTT |
| | | TGATGATTATGCCATGCACTGGGTCCGGCAAGCTCCAGGGAAG |
| | | GGCCTGGAATGGGTCTCAGCTATCACTTGGAATAGTGGTCACA |
| | | TAGACTATGCGGACTCTGTGGAGGGCCGATTCACCATCTCCAG |
| | | AGACAACGCCAAGAACTCCCTGTATCTGCAAATGAACAGTCTG |
| | | AGAGCTGAGGATACGGCCGTATATTACTGTGCGAAAGTCTCGT |
| | | ACCTTAGCACCGCGTCCTCCCTTGACTATTGGGGCCAAGGTACC |
| | | CTGGTCACCGTCTCGAGTGGCGGCGGAGGCAGCGGAGGCGG |
| | | AGGTTCTGGAGGAGGGGGGAGTGACATCCAGATGACCCAGTC |
| | | TCCATCCTCCCTGTCTGCATCTGTAGGGGACAGAGTCACCATCA |
| | | CTTGTCGGGCAAGTCAGGGCATCAGAAATTACTTAGCCTGGTA |
| | | TCAGCAAAAACCAGGGAAAGCCCCTAAGCTCCTGATCTATGCT |
| | | GCATCCACTTTGCAATCAGGGGTCCCATCTCGGTTCAGTGGCA |
| | | GTGGATCTGGGACAGATTTCACTCTCACCATCAGCAGCCTACA |
| | | GCCTGAAGATGTTGCAACTTATTACTGTCAAAGGTATAACCGT |
| | | GCACCGTATACTTTTGGCCAGGGGACCAAGGTGGAAATCAAA |

TABLE 10
Amino acid sequences of mature Quad polypeptides

| SEQ ID NO. | QUAD NO. | AMINO ACID SEQUENCE |
|---|---|---|
| 151 | Quad 51 | EVQLVESGGGLVQPGRSLRLSCAASGFTFDDYAMHWVRQA |
| | | PGKGLEWVSAITWNSGHIDYADSVEGRFTISRDNAKNSLY |
| | | LQMNSLRAEDTAVYYCAKVSYLSTASSLDYWGQGTLVTVS |
| | | SGGGGSGGGGSGGGGSDIQMTQSPSSLSASVGDRVTITCR |
| | | ASQGIRNYLAWYQQKPGKAPKLLIYAASTLQSGVPSRFSG |
| | | SGSGTDFTLTISSLQPEDVATYYCQRYNRAPYTFGQGTKV |
| | | EIKKKKPLDGEYFTLQIRGRERFEMFRELNEALELKDAQA |
| | | GKEPG |
| 152 | Quad 53 Mon | DIVLTQSPAILSASPGEKVTMTCRASSSVNYMDWYQKKPG |
| | | SSPKPWIYATSNLASGVPARFSGSGSGTSYSLTISRVEAE |
| | | DAATYYCQQWSFNPPTFGGGTKLEIKGSTSGGGGSGGGGS |
| | | GGGGSSEVQLQQSGAELVKPGASVKMSCKASGYTFTSYNM |
| | | HWVKQTPGQGLEWIGAIYPGNGDTSYNQKFKGKATLTADK |
| | | SSSTAYMQLSSLTSEDSADYYCARSNYYGSSYWFFDVWGA |
| | | GTTVTVSS |
| 153 | Quad 53 Tet | DIVLTQSPAILSASPGEKVTMTCRASSSVNYMDWYQKKPG |
| | | SSPKPWIYATSNLASGVPARFSGSGSGTSYSLTISRVEAE |
| | | DAATYYCQQWSFNPPTFGGGTKLEIKGSTSGGGGSGGGGS |
| | | GGGGSSEVQLQQSGAELVKPGASVKMSCKASGYTFTSYNM |
| | | HWVKQTPGQGLEWIGAIYPGNGDTSYNQKFKGKATLTADK |
| | | SSSTAYMQLSSLTSEDSADYYCARSNYYGSSYWFFDVWGA |
| | | GTTVTVSSKKKPLDGEYFTLQIRGRERFEMFRELNEALEL |
| | | KDAQAGKEPG |
| 154 | Quad 53 Oct | DIVLTQSPAILSASPGEKVTMTCRASSSVNYMDWYQKKPG |
| | | SSPKPWIYATSNLASGVPARFSGSGSGTSYSLTISRVEAE |
| | | DAATYYCQQWSFNPPTFGGGTKLEIKGSTSGGGGSGGGGS |
| | | GGGGSSEVQLQQSGAELVKPGASVKMSCKASGYTFTSYNM |
| | | HWVKQTPGQGLEWIGAIYPGNGDTSYNQKFKGKATLTADK |
| | | SSSTAYMQLSSLTSEDSADYYCARSNYYGSSYWFFDVWGA |
| | | GTTVTVSSKKKPLDGEYFTLQIRGRERFEMFRELNEALEL |
| | | KDAQAGKEPGDIVLTQSPAILSASPGEKVTMTCRASSSVN |
| | | YMDWYQKKPGSSPKPWIYATSNLASGVPARFSGSGSGTSY |
| | | SLTISRVEAEDAATYYCQQWSFNPPTFGGGTKLEIKGSTS |
| | | GGGGSGGGGSGGGGSSEVQLQQSGAELVKPGASVKMSCKA |
| | | SGYTFTSYNMHWVKQTPGQGLEWIGAIYPGNGDTSYNQKF |
| | | KGKATLTADKSSSTAYMQLSSLTSEDSADYYCARSNYYGS |
| | | SYWFFDVWGAGTTVTVSS |
| 155 | Quad 54 | EVQLVESGGGLVQPGGSLRLSCAASGFTFSDYWMYWVRQA |
| | | PGKGLEWVSEINTNGLITKYPDSVKGRFTISRDNAKNTLY |
| | | LQMNSLRPEDTAVYYCARSPSGFNRGQGTLVTVSSKKKPL |
| | | DGEYFTLQIRGRERFEMFRELNEALELKDAQAGKEPGEVQ |
| | | LVESGGGLVQAGGSLRLSCAASGSSFRFRAMAWYRQAPEK |
| | | QRDFVATINSLGETTYATAVEGRFTISRDNAKNTVYLQMD |
| | | SLKPEDTAVYYCNEPRGNYWGQGTQVTVSS |
| 156 | Quad 55 | EVQLVESGGGLVQPGRSLRLSCAASGFTFDDYAMHWVRQA |
| | | PGKGLEWVSAITWNSGHIDYADSVEGRFTISRDNAKNSLY |
| | | LQMNSLRAEDTAVYYCAKVSYLSTASSLDYWGQGTLVTVS |
| | | SGGGGSGGGGSGGGGSDIQMTQSPSSLSASVGDRVTITCR |
| | | ASQGIRNYLAWYQQKPGKAPKLLIYAASTLQSGVPSRFSG |
| | | SGSGTDFTLTISSLQPEDVATYYCQRYNRAPYTFGQGTKV |
| | | EIKKKKPLDGEYFTLQIRGRERFEMFRELNEALELKDAQA |
| | | GKEPGQVQLVQSGAEVKKPGSSVKVSCKASGYSFTDYHIH |
| | | WVRQAPGQGLEWMGVINPMYGTTDYNQRFKGRVTITADES |
| | | TSTAYMELSSLRSEDTAVYYCARYDYFTGTGVYWGQGTLV |
| | | TVSSGGGGSGGGGSGGGGSDIVMTQTPLSLSVTPGQPASI |
| | | SCRSSRSLVHSRGNTYLHWYLQKPGQSPQLLIYKVSNRFI |
| | | GVPDRFSGSGSGTDFTLKISRVEAEDVGVYYCSQSTHLP |
| | | TFGQGTKLEIK |
| 157 | Quad 56 | EVQLVESGGGLVQPGRSLRLSCAASGFTFDDYAMHWVRQA |
| | | PGKGLEWVSAITWNSGHIDYADSVEGRFTISRDNAKNSLY |
| | | LQMNSLRAEDTAVYYCAKVSYLSIASSLDYWGQGTLVTVS |
| | | SGGGGSGGGGSGGGGSDIQMTQSPSSLSASVGDRVTITCR |
| | | ASQGIRNYLAWYQQKPGKAPKLLIYAASTLQSGVPSRFSG |
| | | SGSGTDFTLTISSLQPEDVATYYCQRYNRAPYTFGQGTKV |
| | | EIKKKKPLDGEYFTLQIRGRERFEMFRELNEALELKDAQA |
| | | GKEPGEVQLVESGGGLVQAGGSLRLSCAASGSSFRFRAMA |
| | | WYRQAPEKQRDFVATINSLGETTYATAVEGRFTISRDNAK |
| | | NTVYLQMDSLKPEDTAVYYCNEPRGNYWGQGTQVTVSS |
| 158 | Quad 57 | EVQLVESGGGLVQAGGSLRLSCAASGSSFRFRAMAWYRQA |
| | | PEKQRDFVATINSLGETTYATAVEGRFTISRDNAKNTVYL |
| | | QMDSLKPEDTAVYYCNEPRGNYWGQGTQVTVSSKKKPLDG |
| | | EYFTLQIRGRERFEMFRELNEALELKDAQAGKEPG |
| 159 | Quad 63 | EVQLVESGGGLVQPGRSLRLSCAASGFTFDDYAMHWVRQA |
| | | PGKGLEWVSAITWNSGHIDYADSVEGRFTISRDNAKNSLY |
| | | LQMNSLRAEDTAVYYCAKVSYLSTASSLDYWGQGTLVTVS |
| | | SGGGGSGGGGSGGGGSDIQMTQSPSSLSASVGDRVTITCR |
| | | ASQGIRNYLAWYQQKPGKAPKLLIYAASTLQSGVPSRFSG |
| | | SGSGTDFTLTISSLQPEDVATYYCQRYNRAPYTFGQGTKV |
| | | EIKGGGGSGGGGSGGGGSKKKPLDGEYFTLQIRGRERFEM |
| | | FRELNEALELKDAQAGKEPG |
| 160 | Quad 64 | DIVLTQSPAILSASPGEKVTMTCRASSSVNYMDWYQKKPG |
| | | SSPKPWIYATSNLASGVPARFSGSGSGTSYSLTISRVEAE |
| | | DAATYYCQQWSFNPPTFGGGTKLEIKGSTSGGGGSGGGGS |
| | | GGGGSSEVQLQQSGAELVKPGASVKMSCKASGYTFTSYNM |
| | | HWVKQTPGQGLEWIGAIYPGNGDTSYNQKFKGKATLTADK |
| | | SSSTAYMQLSSLTSEDSADYYCARSNYYGSSYWFFDVWGA |
| | | GTTVTVSSGGGGSGGGGSGGGGSAPELLGGPSVFLFPPKP |
| | | KDTLMISRIPEVTCVVVDVSHEDPEVKFNWYVDGVEVHNA |
| | | KTKPREEQYNSTYRVVSVLTVLHQDWLNGKEYKCKVSNKA |
| | | LPAPIEKTISKAKGQPREPQVYTLPPSRDELTKNQVSLTC |
| | | LVKGFYPSDIAVEWESNGQPENNYKTTPPVLDSDGSFFLY |
| | | SKLTVDKSRWQQGNVFSCSVMHEALHNHYTQKSLSLSPGK |
| | | KKKPLDGEYFTLQIRGRERFEMFRELNEALELKDAQAGKE |
| | | PG |
| 161 | Quad 65 | DIVLTQSPAILSASPGEKVTMTCRASSSVNYMDWYQKKPG |
| | | SSPKPWIYATSNLASGVPARFSGSGSGTSYSLTISRVEAE |
| | | DAATYYCQQWSFNPPTFGGGTKLEIKGSTSGGGGSGGGGS |
| | | GGGGSSEVQLQQSGAELVKPGASVKMSCKASGYTFTSYNM |
| | | HWVKQTPGQGLEWIGAIYPGNGDTSYNQKFKGKATLTADK |
| | | SSSTAYMQLSSLTSEDSADYYCARSNYYGSSYWFFDVWGA |
| | | GTTVTVSSKKKPLDGEYFTLQIRGRERFEMFRELNEALEL |
| | | KDAQAGKEPGAPELLGGPSVFLFPPKPKDTLMISRTPEVT |
| | | CVVVDVSHEDPEVKFNWYVDGVEVHNAKTKPREEQYNSTY |
| | | RVVSVLTVLHQDWLNGKEYKCKVSNKALPAPIEKTISKAK |
| | | GQPREPQVYTLPPSRDELTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| 162 | W51ScFv | EVQLVESGGGLVQPGRSLRLSCAASGFTFDDYAMHWVRQA |
| | | PGKGLEWVSAITWNSGHIDYADSVEGRFTISRDNAKNSLY |
| | | LQMNSLRAEDTAVYYCAKVSYLSTASSLDYWGQGTLVTVS |
| | | SGGGGSGGGGSGGGGSDIQMTQSPSSLSASVGDRVTITCR |
| | | ASQGIRNYLAWYQQKPGKAPKLLIYAASTLQSGVPSRFSG |
| | | SGSGTDFTLTISSLQPEDVATYYCQRYNRAPYTFGQGTKV |
| | | EIK |

TABLE 11
ED50 of Anti-TNFa Molecules

| Anti-TNFa | ED50 (pM) |
|---|---|
| Quad 51 | 41 |
| Humira | 107 |
| W51ScFv | 246 |

TABLE 12(a)
Amino acid sequence of the hinge region
of human IgG isotypes & sequences

| | | | Lower |
|---|---|---|---|
| | Upper | Core | Hinge/ |
| Isotype | Hinge | Hinge | CH2 Region |
| Human IgG1 | EPKSCDKTHT | CPPCP | APELLGGPSV |
| Human IgG2 | ERKCCVE | CPPCP | APPVAGPSV |
| Human IgG3 | ELKTPLGDTTHT | CPRCP | APELLGGPSV |
| (exon 1) | | | |
| Human IgG3 | EPKSCDTPPP | CPRCP | APELLGGPSV |
| (exons 2, | | | |
| 3, 4) | | | |
| Human IgG4 | ESKYGPP | CPSCP | APEFLGGPSV |

CXXC motif of core hinge is underlined

TABLE 12(b)
Further Hinge Sequences, Upper and
Core Regions

| | |
|---|---|
| SEQ ID NO 163: | APELLGGPSV |
| SEQ ID NO 164: | PAPELLGGPSV |
| SEQ ID NO 165: | APPVAGPSV |
| SEQ ID NO 166: | PAPPVAGPSV |
| SEQ ID NO 167: | EPKSCDKTHTPAPELLGGPSV |
| SEQ ID NO 168: | EPKSCDKTHTAPELLGGPSV |
| SEQ ID NO 169: | ERKCCVEPAPPVAGPSV |
| SEQ ID NO 170: | ERKCCVEAPPVAGPSV |
| SEQ ID NO 171: | ELKTPLGDTTHTPAPELLGGPSV |
| SEQ ID NO 172: | ELKTPLGDTTHTPAPELLGGPSV |
| SEQ ID NO 173: | EPKSCDTPPPPAPELLGGPSV |
| SEQ ID NO 174: | EPKSCDTPPPAPELLGGPSV |
| SEQ ID NO 175: | APEFLGGPSV |
| SEQ ID NO 176: | PAPEFLGGPSV |
| SEQ ID NO 177: | ESKYGPPPAPEFLGGPSV |
| SEQ ID NO 178: | ESKYGPPAPEFLGGPSV |
| SEQ ID NO 179: | Humira light Chain |
| | (see Table 18) |
| SEQ ID NO 180: | CPPC |
| SEQ ID NO 181: | CPRC |
| SEQ ID NO 182: | CPSC |
| SEQ ID NO 183: | EPKSCDKTHT |
| SEQ ID NO 184: | ERKCCVE |
| SEQ ID NO 185: | ELKTPLGDTTHT |
| SEQ ID NO 186: | EPKSCDTPPP |
| SEQ ID NO 187: | ESKYGPP |

TABLE 13
UniProt accession numbers of proteins containing p53 tetramerisation domains (TD).
The amino acid sequence position of the TD domain for each protein is also indicated (ie, for
the first entry, the TD sequence is amino acid residues 345 to (and including) 383 of the
protein disclosed in UniProt with accession number A0A024R4C3).

| | | |
|---|---|---|
| >A0A024R4C3/345-383 | >A0A2I3GNV1/391-430 | >F7FHP5/462-501 |
| >A0A059UCX8/312-350 | >A0A2I3GRR4/391-430 | >F7FN59/397-436 |
| >A0A060XH67/46-81 | >A0A2I3GX41/212-251 | >F7FN64/397-436 |
| >A0A060XN97/313-350 | >A0A2I3H7C7/391-430 | >F7FN69/212-251 |
| >A0A060Z608/94-130 | >A0A2I3HHX4/160-198 | >F7PQR2/76-114 |
| >A0A075BAE6/344-382 | >A0A2I3HR34/391-430 | >F7GA34/345-383 |
| >A0A087R7Z2/374-413 | >A0A2I3HZV6/292-331 | >F7GA47/345-383 |
| >A0A087VQ67/198-237 | >A0A2I3M190/296-334 | >F7GA51/296-334 |
| >A0A087WZU8/308-346 | >A0A2I3M2V8/212-251 | >F7GBG3/297-336 |
| >A0A087X1Q1/160-198 | >A0A2I3M6G1/345-383 | >F7GBG7/297-336 |
| >A0A087X5S1/385-424 | >A0A2I3M9J9/391-430 | >F7GBH1/374-413 |
| >A0A087XP53/284-317 | >A0A2I3MCI3/328-366 | >F7GEP9/271-310 |
| >A0A087XYP3/293-330 | >A0A2I3MEJ7/347-386 | >F7GNX0/321-359 |
| >A0A088DIC9/324-356 | >A0A2I3MFF3/298-336 | >F7GP14/311-349 |
| >A0A091CJU7/316-354 | >A0A2I3MKF2/391-430 | >F7HW67/292-335 |
| >A0A091DI49/487-525 | >A0A2I3MNM6/397-436 | >F71720/352-391 |
| >A0A091DSC7/272-311 | >A0A2I3MNQ1/292-331 | >F7I9C6/352-391 |
| >A0A091EA06/374-413 | >A0A2I3N2E1/291-329 | >F719D3/297-336 |
| >A0A091G407/347-386 | >A0A2I3N5A2/345-383 | >F7I9E9/212-251 |
| >A0A091GU41/346-385 | >A0A2I3N736/391-430 | >F7IGK5/391-430 |
| >A0A091H015/371-410 | >A0A2I3N7F5/345-383 | >F8RKR1/315-351 |
| >A0A091H3D1/198-237 | >A0A2I3NGN2/242-280 | >G1K2L5/294-334 |
| >A0A091HZ84/371-410 | >A0A2I3RBD8/296-335 | >G1L1S8/391-430 |
| >A0A091IJF7/347-386 | >A0A2I3REG9/297-336 | >G1LRQ8/345-384 |
| >A0A091JY20/198-237 | >A0A2I3RJD2/345-383 | >G1MEP6/307-345 |
| >A0A091KM07/374-413 | >A0A2I3RLD5/297-336 | >G1MS14/371-410 |
| >A0A091LTD4/198-237 | >A0A2I3RMS1/296-334 | >G1MS29/207-246 |
| >A0A091LVH3/198-237 | >A0A2I3RNZ6/375-414 | >G1N6I1/293-332 |
| >A0A091M6B6/347-386 | >A0A2I3RVP4/212-251 | >G1PSQ3/371-410 |
| >A0A091MGV6/198-237 | >A0A2I3S1V8/314-352 | >G1PZ51/314-345 |
| >A0A091NGU6/286-325 | >A0A2I3SJI2/345-383 | >G1R4S5/297-336 |
| >A0A091PJY2/198-237 | >A0A2I3SPJ9/345-383 | >G1RF61/287-325 |
| >A0A091PKU4/198-237 | >A0A2I3SZA7/345-383 | >G1SEU0/317-355 |
| >A0A091QIR4/198-237 | >A0A2I3SZK4/391-430 | >G1TBP7/297-336 |
| >A0A091R450/198-237 | >A0A2I3T0Z7/391-430 | >G1U940/294-332 |
| >A0A091RK28/198-237 | >A0A2I4ANK8/293-330 | >G2HEX8/238-276 |
| >A0A091TQD5/198-237 | >A0A2I4ANL9/404-441 | >G3GT84/353-392 |
| >A0A091UUY0/347-386 | >A0A2I4ANN9/379-416 | >G3GY68/74-112 |
| >A0A091VBV4/374-413 | >A0A2I4ANQ2/291-328 | >G3IHF1/290-328 |
| >A0A091VW97/391-430 | >A0A214C4P9/303-338 | >G3NTK8/356-393 |
| >A0A093BPE2/104-143 | >A0A2I4C876/81-120 | >G3PK82/295-334 |
| >A0A093BXJ6/60-99 | >A0A2I4CET7/295-334 | >G3Q6V4/297-340 |
| >A0A093E9I7/198-237 | >A0A2I4CET8/386-425 | >G3Q6V7/284-327 |
| >A0A093FLV6/198-237 | >A0A2I4CEU0/291-330 | >G3QQY2/297-336 |
| >A0A093GLW0/374-413 | >A0A2I4CEU4/382-421 | >G3R2U9/319-357 |
| >A0A093HAC9/347-386 | >A0A2J8K8U5/345-383 | >G3RHQ4/345-383 |
| >A0A093HBT9/198-237 | >A0A2J8K8U6/345-383 | >G3RXL5/212-251 |
| >A0A093IHW1/104-143 | >A0A2J8K8U7/345-383 | >G3SZA7/318-357 |
| >A0A093JJ94/283-322 | >A0A2J8K8U9/296-334 | >G3T035/318-356 |
| >A0A093K3B8/391-430 | >A0A2J8K8V2/274-312 | >G3TS21/289-328 |
| >A0A093NBQ1/374-413 | >A0A2J8K8V5/296-334 | >G3TT62/376-415 |
| >A0A093PYK4/391-430 | >A0A2J8K8X6/345-383 | >G3TYZ8/391-430 |
| >A0A093R114/347-386 | >A0A2J8K8Z2/296-334 | >G3U6D1/290-329 |
| >A0A093T5L0/283-322 | >A0A2J8K8Z8/345-383 | >G3U6U6/293-332 |
| >A0A094L4V0/283-322 | >A0A2J8KPA8/308-346 | >G3UAZ0/290-329 |
| >A0A094LB61/374-413 | >A0A2J8KPB0/280-318 | >G3UDE4/288-327 |
| >A0A094MNK3/330-369 | >A0A2J8KPB3/319-357 | >G3UHE5/293-332 |
| >A0A096MBY3/303-335 | >A0A2J8KPB5/160-198 | >G3U157/286-325 |
| >A0A096MTM8/391-430 | >A0A2J8M649/391-430 | >G3UJO0/286-325 |
| >A0A096N540/345-383 | >A0A2J8M650/391-430 | >G3UK14/298-332 |
| >A0A096P2R8/319-357 | >A0A2J8M651/293-332 | >G3ULT4/293-332 |
| >A0A099ZPQ3/374-413 | >A0A2J8M652/391-430 | >G3UYS2/296-334 |
| >A0A099ZQ65/347-386 | >A0A2J8M655/391-430 | >G3VTK8/202-241 |
| >A0A0A0AG46/374-413 | >A0A2J8M660/212-251 | >G3VZR1/347-386 |
| >A0A0A0AU13/347-386 | >A0A2J8M663/297-336 | >G3WS63/296-334 |
| >A0A0B4VEY6/209-249 | >A0A2J8M665/297-336 | >G3X6J7/346-385 |
| >A0A0B4VFS7/181-221 | >A0A2J8M666/297-336 | >G5B5D6/317-355 |
| >A0A0B7AXG3/73-113 | >A0A2J8M667/387-426 | >G5CA58/391-430 |
| >A0A0B7C164/5-45 | >A0A2J8RWD8/319-357 | >G5CBI6/345-383 |
| >A0A0C4DFW9/296-334 | >A0A2J8RWE9/308-346 | >G7MGI1/345-383 |
| >A0A0C5DEW5/307-345 | >A0A2J8RWG5/160-198 | >G7MK85/391-430 |
| >A0A0C5DID8/307-345 | >A0A2J8RWJ2/254-292 | >G7NTA7/345-383 |
| >A0A0C5E1H5/307-345 | >A0A2J8RWJ8/280-318 | >G7NYP4/391-430 |
| >A0A0D2X0F0/498-530 | >A0A2J8URM7/345-383 | >G7PTI9/319-357 |
| >A0A0D9R9K6/211-250 | >A0A2J8URN5/296-334 | >G9J1L8/307-346 |
| >A0A0D9RG12/319-357 | >A0A2J8URN6/345-383 | >G9J1L9/416-454 |
| >A0A0D9S8U4/345-383 | >A0A2J8URP0/296-334 | >G9J1M0/350-389 |
| >A0A0F6QMK2/371-410 | >A0A2J8URP1/345-383 | >G9KUQ2/301-339 |
| >A0A0F6QNH5/371-410 | >A0A2J8URP3/296-334 | >H0VHZ7/296-334 |
| >A0A0F6QPP8/371-410 | >A0A2J8URQ1/274-312 | >H0VRT2/391-430 |
| >A0A0F6QPQ1/287-326 | >A0A2J8URQ6/345-383 | >H0WNK7/294-333 |
| >A0A0F6T5N8/287-326 | >A0A2J8URQ8/345-383 | >H0WTD3/348-386 |
| >A0A0F6T5N9/287-326 | >A0A2J8WHZ2/391-430 | >H0XGB0/319-357 |
| >A0A0F7YYL7/283-323 | >A0A2J8WHZ4/391-430 | >H0YX04/347-386 |
| >A0A0F7YYL8/287-328 | >A0A2J8WHZ7/297-336 | >H0ZGH4/391-430 |
| >A0A0F7YYP8/275-315 | >A0A2J8WI03/391-430 | >H2EHT1/280-318 |
| >A0A0F7YYQ1/283-323 | >A0A2J8WI05/387-426 | >H2LHQ7/336-373 |
| >A0A0F7YYT3/270-310 | >A0A2J8WI12/297-336 | >H2LPP5/296-337 |
| >A0A0F7YYT4/350-391 | >A0A2J8WI13/391-430 | >H2LPP7/245-286 |
| >A0A0F7YYT5/288-328 | >A0A2J8WI16/212-251 | >H2MLN6/374-413 |
| >A0A0F7YYU0/270-310 | >A0A2J8WI18/297-336 | >H2MLN7/292-331 |
| >A0A0F7YYU1/287-328 | >A0A2J8WI20/297-336 | >H2MLN8/292-331 |
| >A0A0F7YYU2/288-328 | >A0A2J8W122/293-332 | >H2MLP0/296-335 |
| >A0A0F7YYU7/275-315 | >A0A2K5DGQ7/303-331 | >H2N9D2/244-269 |
| >A0A0F7YYU8/350-391 | >A0A2K5DXG0/283-320 | >H2NSL2/319-357 |
| >A0A0F7YYU9/356-396 | >A0A2K5DXH0/149-176 | >H2PCB7/391-430 |
| >A0A0F8AIC7/289-331 | >A0A2K5E0Y4/345-383 | >H2PXV6/345-383 |
| >A0A0F8AX61/164-203 | >A0A2K5E0Y9/345-383 | >H2QC53/319-357 |
| >A0A0F8CAV8/414-453 | >A0A2K5E0Z8/296-334 | >H2QNY5/391-430 |
| >A0A0G2KB75/105-133 | >A0A2K5E105/345-383 | >H2S6K3/392-431 |
| >A0A0G2KBB2/102-133 | >A0A2K5ESD1/391-430 | >H2S6K4/298-337 |
| >A0A0H3U6U5/312-348 | >A0A2K5ESD6/217-256 | >H2S6K5/273-312 |
| >A0A0K1TP12/317-355 | >A0A2K5ESG6/297-336 | >H2S6K6/294-333 |
| >A0A0L0G1F4/366-392 | >A0A2K5ESH6/296-335 | >H2S6K7/308-347 |
| >A0A0L8G262/360-396 | >A0A2K5ESI5/391-430 | >H2U133/246-281 |
| >A0A0N7FDT7/312-348 | >A0A2K5ESI9/391-430 | >H2U134/291-326 |
| >A0A0N8JWU8/295-331 | >A0A2K5ESJ1/391-430 | >H2U135/287-322 |
| >A0A0P6J3J0/297-336 | >A0A2K5ESJ8/377-416 | >H2UMJ4/356-393 |
| >A0A0P7UTD5/303-342 | >A0A2K5ESK8/212-251 | >H2UMJ5/351-388 |
| >A0A0P7Y6W7/139-178 | >A0A2K5F3V2/255-292 | >H2UMJ6/286-323 |
| >A0A0Q3M6G6/462-501 | >A0A2K5IAQ7/345-383 | >H2UMJ7/282-319 |
| >A0A0Q3T504/345-384 | >A0A2K5IAT1/296-334 | >H2ZGE3/352-384 |
| >A0A0R2XFU5/51-67 | >A0A2K5IAT6/299-337 | >H2ZH73/107-145 |
| >A0A0R4IHL8/385-424 | >A0A2K5IAV0/242-280 | >H2ZH74/405-443 |
| >A0A0R4IZ80/348-387 | >A0A2K5IAW1/291-329 | >H2ZH75/337-375 |
| >A0A0S2Z4N5/391-430 | >A0A2K5IB21/345-383 | >H3B1Z4/325-365 |
| >A0A0S2Z4N6/387-426 | >A0A2K51B95/345-383 | >H3B2L6/290-329 |
| >A0A0S7IX91/41-80 | >A0A2K5JJ67/319-357 | >H3BII1/352-391 |
| >A0A0S7K6D3/282-314 | >A0A2K5JJ94/322-360 | >H3CXQ0/301-337 |
| >A0A0U1RQC9/280-318 | >A0A2K5K821/391-430 | >H3D350/357-394 |
| >A0A0U1XP71/182-219 | >A0A2K5K823/391-430 | >H3D8D5/375-414 |
| >A0A0U3KDC1/470-508 | >A0A2K5K834/297-336 | >H6U5S2/319-357 |
| >A0A0U4B546/314-351 | >A0A2K5K835/365-404 | >H6U5S3/319-357 |
| >A0A0U4D4F9/352-387 | >A0A2K5K845/296-335 | >H9FFS1/103-141 |
| >A0A141PNN3/293-332 | >A0A2K5K849/296-335 | >H9G3N4/212-237 |
| >A0A141PNN4/208-247 | >A0A2K5MIE5/212-251 | >H9G5L7/345-384 |
| >A0A142GR17/315-351 | >A0A2K5MIF0/292-331 | >H9GVA1/99-126 |
| >A0A146NLN6/277-313 | >A0A2K5MIF3/347-386 | >H9N2D2/378-416 |
| >A0A146P3Z1/292-328 | >A0A2K5MIG1/392-431 | >H9N2D3/378-416 |
| >A0A146VRU0/254-290 | >A0A2K5MIH1/391-430 | >H9ZEQ2/293-332 |
| >A0A146VSL6/290-325 | >A0A2K5MIH4/391-430 | >I3J187/356-394 |
| >A0A146X8K2/291-330 | >A0A2K5MIH6/297-336 | >I3KRX9/307-343 |
| >A0A146X8M0/387-426 | >A0A2K5MIP6/391-430 | >I3KT80/392-431 |
| >A0A146X8S3/295-334 | >A0A2K5MWU1/345-383 | >I3LA53/311-348 |
| >A0A146X9W6/383-422 | >A0A2K5MWV4/345-383 | >I3LPD4/391-430 |
| >A0A146XA86/291-330 | >A0A2K5MWV7/345-383 | >I3LPE8/346-385 |
| >A0A146XAK7/291-330 | >A0A2K5MWW4/296-334 | >I3MLP5/391-430 |
| >A0A146XAZ2/383-422 | >A0A2K5MWX3/345-383 | >I3N5N2/317-355 |
| >A0A146XBF3/383-422 | >A0A2K5MX14/242-280 | >I3NDP5/343-381 |
| >A0A146Z8H7/322-359 | >A0A2K5MXC3/345-383 | >I7HIK9/313-350 |
| >A0A146Z814/293-330 | >A0A2K5MXD2/296-334 | >K1RC48/553-594 |
| >A0A146Z8Q2/224-261 | >A0A2K5N3I9/319-357 | >K7F8T8/345-384 |
| >A0A146Z9Z7/379-416 | >A0A2K5N3Z0/328-366 | >K7G3P4/312-351 |
| >A0A147ACK6/387-426 | >A0A2K5QQH4/391-430 | >K7GCN3/282-321 |
| >A0A147AT87/94-124 | >A0A2K5QQI1/382-421 | >K7PPA8/319-357 |
| >A0A147B4U1/313-350 | >A0A2K5QQI6/391-430 | >K7PPU4/319-357 |
| >A0A151MM54/441-480 | >A0A2K5QQJ5/297-336 | >K9KFA7/17-55 |
| >A0A151MMC3/437-476 | >A0A2K5QQM5/297-336 | >L5JRP0/371-410 |
| >A0A151MW63/306-341 | >A0A2K5QQM6/296-335 | >L5JZ91/308-342 |
| >A0A151N3K6/400-439 | >A0A2K5QQP2/391-430 | >L5KJ90/409-448 |
| >A0A151N3K8/361-400 | >A0A2K5QQU5/212-251 | >L5LRF0/352-391 |
| >A0A151N445/377-416 | >A0A2K5QR20/235-274 | >L5M0C7/325-357 |
| >A0A158SIS7/313-350 | >A0A2K5QYI7/345-383 | >L5MJN5/108-133 |
| >A0A167VDT2/391-430 | >A0A2K5QYL2/345-383 | >L7N1B2/317-349 |
| >A0A172Q425/316-353 | >A0A2K5QYN8/345-383 | >L7NCR5/316-355 |
| >A0A1A6HG68/222-260 | >A0A2K5QYP8/296-334 | >L7X0Y9/317-356 |
| >A0A1A7X7J4/307-348 | >A0A2K5RMZ8/321-359 | >L7X1P3/317-356 |
| >A0A1A7ZCT3/81-118 | >A0A2K5RN37/282-320 | >L7X447/317-354 |
| >A0A1A8AZS7/38-77 | >A0A2K5TIV2/415-453 | >L8IIU4/371-410 |
| >A0A1A8C8M1/255-293 | >A0A2K5VC74/297-336 | >L8IPI8/312-348 |
| >A0A1A8D5F3/314-349 | >A0A2K5VC97/292-331 | >L8IY21/346-385 |
| >A0A1A8I4T3/1-29 | >A0A2K5VCB4/212-251 | >L9JK63/422-461 |
| >A0A1A81W64/291-330 | >A0A2K5VCD2/397-436 | >L9KM90/323-358 |
| >A0A1A8JIA0/314-349 | >A0A2K5VCE6/297-336 | >M0R497/209-247 |
| >A0A1A8JZF7/291-330 | >A0A2K5VCE8/391-430 | >M3VVE4/347-386 |
| >A0A1A8N999/312-347 | >A0A2K5VCF3/391-430 | >M3WFF4/461-500 |
| >A0A1A8P8L4/312-345 | >A0A2K5V1Y5/345-383 | >M3XIT7/352-391 |
| >A0A1A8UK50/314-349 | >A0A2K5VJ29/242-280 | >M3MVF7/297-336 |
| >A0A1B1CUP8/290-331 | >A0A2K5VJ84/345-383 | >M3Y7P2/416-455 |
| >A0A1B2TT48/312-349 | >A0A2K5VJA4/345-383 | >M3YC88/301-339 |
| >A0A1B8Y052/1-27 | >A0A2K5VJA9/296-334 | >M4APR6/291-330 |
| >MAIDSQH87/284-322 | >A0A2K5VJB1/345-383 | >M4ASF8/203-240 |
| >A0A117Q490/328-366 | >A0A2K5VJC2/291-329 | >M7AN48/275-314 |
| >A0A1L8FGI1/429-468 | >A0A2K5WN47/317-355 | >M7CAH2/325-364 |
| >A0A1L8FMK1/465-504 | >A0A2K5XAH3/306-345 | >O09185/319-357 |
| >A0A1L8G3W1/293-332 | >A0A2K5XAK1/306-345 | >O12946/301-329 |
| >A0A1L8G9P1/293-332 | >A0A2K5XAR2/306-345 | >O15350/345-383 |
| >A0A1L8H4Q1/128-170 | >A0A2K5XAR7/212-251 | >O36006/317-355 |
| >A0A1S2ZRH2/303-341 | >A0A2K5XAS2/306-345 | >O57538/285-316 |
| >A0A1S2ZWK7/405-444 | >A0A2K5XTB8/345-383 | >O70366/316-353 |
| >A0A1S2ZWM0/405-444 | >A0A2K5XTC3/296-334 | >O88898/391-430 |
| >A0A1S2ZWM2/297-336 | >A0A2K5XTC5/345-383 | >O93379/295-334 |
| >A0A1S2ZWM5/297-336 | >A0A2K5XTC7/291-329 | >P02340/313-350 |
| >A0A1S2ZWM8/405-444 | >A0A2K5XTF6/345-383 | >P04637/319-357 |
| >A0A1S2ZWM9/348-387 | >A0A2K5XTG5/345-383 | >P07193/290-333 |
| >A0A1S3A060/343-381 | >A0A2K6AAH4/319-357 | >P10360/300-335 |
| >A0A1S3A061/341-379 | >A0A2K6AAI9/328-366 | >P10361/317-354 |
| >A0A1S3A068/343-381 | >A0A2K6BU42/347-386 | >P13481/319-357 |
| >A0A1S3EYC1/340-378 | >A0A2K6BU55/391-430 | >P25035/320-355 |
| >A0A1S3FDV5/391-430 | >A0A2K6BU74/397-436 | >P41685/312-350 |
| >A0A1S3FDV9/391-430 | >A0A2K6BU80/212-251 | >P51664/308-345 |
| >A0A1S3FDW4/293-332 | >A0A2K6BUA9/292-331 | >P56423/319-357 |
| >A0A1S3FE88/293-332 | >A0A2K6BUB5/391-430 | >P56424/319-357 |
| >A0A1S3FE98/272-311 | >A0A2K6BUD7/391-430 | >P61260/319-357 |
| >A0A1S3FET2/297-336 | >A0A2K6BUE7/297-336 | >P67938/312-348 |
| >A0A1S3FET7/297-336 | >A0A2K6CLT3/345-383 | >P67939/312-348 |
| >A0A1S3FEU1/293-332 | >A0A2K6CLV3/345-383 | >P79734/293-333 |
| >A0A1S3FFH4/297-336 | >A0A2K6CLW2/298-336 | >P79820/291-334 |
| >A0A1S3FFH7/387-426 | >A0A2K6CLW4/345-383 | >P89002/304-341 |
| >A0A1S3FFI4/297-336 | >A0A2K6CLX0/296-334 | >P89003/212-249 |
| >A0A1S3FFS7/391-430 | >A0A2K6CLX4/291-329 | >P89004/212-229 |
| >A0A1S3FFT3/297-336 | >A0A2K6CLX8/345-383 | >P90332/212-249 |
| >A0A1S3G157/345-383 | >A0A2K6CLY4/242-280 | >Q00366/322-359 |
| >A0A1S3GID5/296-334 | >A0A2K6FGK8/283-321 | >Q0GGT2/405-444 |
| >A0A1S3GJQ8/345-383 | >A0A2K6FGL2/296-334 | >Q0GMA7/311-349 |
| >A0A1S3GJR4/343-381 | >A0A2K6FGL6/283-321 | >Q0H3B6/346-384 |
| >A0A1S31134/365-404 | >A0A2K6FGL7/283-321 | >Q0H3B7/346-384 |
| >A0A1S311C6/286-325 | >A0A2K6GXH2/391-430 | >Q0JRM9/357-397 |
| >A0A1S31K29/371-410 | >A0A2K6GXH6/391-430 | >Q0Z9Z4/312-348 |
| >A0A1S3MEP3/276-315 | >A0A2K6GXJ5/391-430 | >Q1AMZ5/287-326 |
| >A0A1S3MEY9/375-414 | >A0A2K6GXK1/202-241 | >Q1AMZ6/409-448 |
| >A0A1S3MEZ2/352-391 | >A0A2K6GXL6/297-336 | >Q1AMZ7/287-326 |
| >A0A1S3MFE6/292-331 | >A0A2K6GXM1/291-330 | >Q1AMZ8/409-448 |
| >A0A1S3MQ52/298-337 | >A0A2K6GXQ4/212-251 | >Q1MSX0/308-346 |
| >A0A1S3MQ93/294-333 | >A0A2K6GXQ9/391-430 | >Q1XDZ3/350-391 |
| >A0A1S3MQE2/298-337 | >A0A2K6GZU4/311-349 | >Q27918/143-184 |
| >A0A1S3MQI7/294-333 | >A0A2K6H000/279-317 | >Q27937/344-382 |
| >A0A1S3MRC1/298-337 | >A0A2K6KG94/374-413 | >Q29475/212-250 |
| >A0A1S3N1A7/158-197 | >A0A2K6KGA5/391-430 | >Q29537/307-345 |
| >A0A1S3N1A8/164-203 | >A0A2K6KGA7/391-430 | >Q2XSC7/319-357 |
| >A0A1S3N1S0/164-203 | >A0A2K6KGB7/390-429 | >Q3UGQ1/316-353 |
| >A0A1S3N6N6/317-355 | >A0A2K6KGB9/297-336 | >Q3UVI3/293-332 |
| >A0A1S3N6V0/295-333 | >A0A2K6KGC8/297-336 | >Q4H2Z8/372-406 |
| >A0A1S3RGF1/354-389 | >A0A2K6KGD2/391-430 | >Q4H2Z9/104-147 |
| >A0A1S3RHN6/275-311 | >A0A2K6KGD4/212-251 | >Q4H300/332-375 |
| >A0A1S3SD25/319-356 | >A0A2K6KGE6/292-331 | >Q4H301/406-449 |
| >A0A1S3WGN9/343-381 | >A0A2K6KGG2/272-311 | >Q4S122/283-322 |
| >A0A1S6QMR1/317-356 | >A0A2K6L161/345-383 | >Q4S837/349-386 |
| >A0A1U7Q2G4/322-359 | >A0A2K6L165/345-383 | >Q4VR33/80-119 |
| >A0A1U7QGA5/387-426 | >A0A2K6L172/296-334 | >Q502Q9/294-334 |
| >A0A1U7QP43/391-430 | >A0A2K6L176/242-280 | >Q533U3/152-189 |
| >A0A1U7QRT0/391-430 | >A0A2K6L183/345-383 | >Q535D5/75-114 |
| >A0A1U7QUI2/168-206 | >A0A2K6L184/345-383 | >Q535D6/75-114 |
| >A0A1U7RA51/361-400 | >A0A2K6L191/291-329 | >Q539B9/350-389 |
| >A0A1U7RF06/348-387 | >A0A2K6LYM5/319-357 | >Q53CG6/350-389 |
| >A0A1U7RM18/352-391 | >A0A2K6NHJ2/370-409 | >Q53GA5/84-122 |
| >A0A1U7SJ11/303-338 | >A0A2K6NHJ7/297-336 | >Q549C9/316-353 |
| >A0A1U7U5H4/314-352 | >A0A2K6NHJ9/357-396 | >Q569E5/297-336 |
| >A0A1U7UA19/343-381 | >A0A2K6NHK0/212-251 | >Q5CZX0/293-332 |
| >A0A1U7UBS6/391-430 | >A0A2K6NHK3/292-331 | >Q5KQU6/352-391 |
| >A0A1U7UDX9/345-383 | >A0A2K6NHK6/370-409 | >Q5U0E4/319-357 |
| >A0A1U7UHU2/391-430 | >A0A2K6NHK7/370-409 | >Q5XHJ3/290-332 |
| >A0A1U7UHU7/297-336 | >A0A2K6NHL2/370-409 | >Q68VB0/317-355 |
| >A0A1U7ULZ4/391-430 | >A0A2K6QDI6/319-357 | >Q6DG24/292-331 |
| >A0A1U7UM51/345-383 | >A0A2K6RQ20/345-383 | >Q6NTF1/290-332 |
| >A0A1U7UNN1/345-383 | >A0A2K6RQ27/345-383 | >Q6PX73/87-126 |
| >A0A1U7UWH9/297-336 | >A0A2K6RQ35/345-383 | >Q6PX74/81-120 |
| >A0A1U8BI05/322-359 | >A0A2K6RQ45/299-337 | >Q6TDG9/40-76 |
| >A0A1U8C2W8/268-307 | >A0A2K6RQ46/296-334 | >Q6UNX2/352-391 |
| >A0A1U8DCG3/297-336 | >A0A2K6RQ50/242-280 | >Q6WG19/347-388 |
| >A0A1U9W5F4/312-349 | >A0A2K6RQ51/345-383 | >Q6WG20/347-388 |
| >A0A1V4KMC3/387-426 | >A0A2K6SLB6/160-198 | >Q71TU9/290-333 |
| >A0A1V4KMH2/387-426 | >A0A2K6SLC5/282-320 | >Q7JP12/135-176 |
| >A0A1V4KNI0/361-400 | >A0A2K6ST65/345-383 | >Q7JP13/344-382 |
| >A0A1V9Y190/398-426 | >A0A2K6ST84/296-334 | >Q7T1D0/290-332 |
| >A0A1W4Y536/311-346 | >A0A2K6ST97/345-383 | >Q801Z7/352-391 |
| >A0A1W4YEN5/186-221 | >A0A2K6STA2/345-383 | >Q80ZA1/316-353 |
| >A0A1W4YF00/386-421 | >A0A2K6UR03/391-430 | >Q8HY32/191-229 |
| >A0A1W4YKL7/349-388 | >A0A2K6UR22/391-430 | >Q8JFE3/292-331 |
| >A0A1W4YLJ9/309-345 | >A0A2K6UR33/382-421 | >Q8JHZ5/296-335 |
| >A0A1W4YUN4/291-330 | >A0A2K6UR38/297-336 | >Q8JHZ6/292-331 |
| >A0A1W4YUY5/350-389 | >A0A2K6UR78/212-251 | >Q8SPZ3/313-351 |
| >A0A1W5AAA0/298-337 | >A0A2K6UR79/297-336 | >Q8T7V3/351-392 |
| >A0A1W5AAA4/294-333 | >A0A2K6UR80/391-430 | >Q91XH8/316-353 |
| >A0A1W5AH30/298-337 | >A0A2K6URF4/272-311 | >Q920Y0/316-353 |
| >A0A1W5AIR4/298-337 | >A5JSV4/301-336 | >Q92143/285-316 |
| >A0A1W5BGP0/491-534 | >A7S4C8/153-191 | >Q95330/317-355 |
| >A0A1W5BI93/406-449 | >A7YYJ7/292-331 | >Q98SW0/293-332 |
| >A0A1W5BJU4/491-534 | >A8DPD6/370-408 | >Q9D6A3/2-30 |
| >A0A1W5BMM1/491-534 | >A9XR54/314-345 | >Q9DEC7/293-332 |
| >A0A1W5BP67/400-443 | >B0R0M3/289-329 | >Q9EPP9/105-133 |
| >A0A1W5W828/297-335 | >B0S576/352-391 | >Q9H3D4/391-430 |
| >A0A1W6BQF7/287-324 | >B0S577/352-391 | >Q91880/99-135 |
| >A0A1Z5LG07/266-293 | >B3GGC4/344-383 | >Q91885/99-135 |
| >A0A210QTK4/425-465 | >B3GGC5/344-383 | >Q9JJP2/337-375 |
| >A0A212CRH3/183-222 | >B3GGC6/183-222 | >Q9JJP6/391-430 |
| >A0A212D9G7/220-245 | >B3RZS6/383-422 | >Q9N252/308-345 |
| >A0A218MZN0/291-333 | >B3TLB0/310-349 | >Q9NGC7/358-399 |
| >A0A218UKS0/168-207 | >B4DMH2/294-332 | >Q9NGC8/358-399 |
| >A0A226MN96/387-426 | >B4DNI2/309-347 | >Q9TTA1/319-357 |
| >A0A226PF87/352-391 | >B5TJK8/318-354 | >Q9TUB2/312-349 |
| >A0A250YHC8/325-363 | >B6E4X6/319-357 | >Q9TUX4/202-240 |
| >A0A286XAH3/297-336 | >B7Z8X6/297-336 | >Q9W664/353-392 |
| >A0A286XCS8/296-334 | >B8X347/358-399 | >Q9W678/289-330 |
| >A0A286XNY6/391-430 | >B8X348/358-399 | >Q9W679/304-340 |
| >A0A286XRB4/296-334 | >C0H8X1/320-355 | >Q9WUR6/317-355 |
| >A0A286XSJ9/347-385 | >C0PUM1/309-346 | >Q9XSK8/345-383 |
| >A0A286W13/349-387 | >C3VC56/308-345 | >R0KB24/324-363 |
| >A0A286Y3E5/391-430 | >C3XPU2/393-433 | >R0KYI4/374-413 |
| >A0A287B319/368-405 | >C3YXH3/317-348 | >R4JHU7/351-390 |
| >A0A287BET2/370-409 | >C3ZIW1/75-106 | >R7UHV7/351-390 |
| >A0A287BN74/347-386 | >C6ERD9/314-349 | >R9TM96/107-146 |
| >A0A287D2W7/297-336 | >C9D7C9/297-336 | >R9XXS5/314-350 |
| >A0A287D527/387-426 | >C9D7D0/297-336 | >S4RR03/224-261 |
| >A0A287D7P9/391-430 | >D1LXA7/297-335 | >S7NG87/345-377 |
| >A0A293MX11/320-357 | >D2H062/371-410 | >S7NJH9/356-395 |
| >A0A2B4RUK1/425-463 | >D2HC09/337-376 | >S9WLV0/1-26 |
| >A0A2C9K413/338-378 | >D2HPX0/307-345 | >S9WR65/202-241 |
| >A0A2C9K4K6/482-522 | >D4AA88/390-428 | >S9YSN3/345-372 |
| >A0A2D0QVX7/291-330 | >D5KTJ0/307-344 | >T0MFN1/322-360 |
| >A0A2D0QXV1/297-336 | >D7PQW0/201-241 | >T1EZJ4/247-278 |
| >A0A2D0QYJ0/372-411 | >E3U906/319-357 | >U3CI16/296-334 |
| >A0A2D0RBJ9/317-353 | >E5RMA8/319-357 | >U3D146/321-359 |
| >A0A2D0RXW5/294-333 | >E7F1Y6/307-345 | >U3D342/293-332 |
| >A0A2D0RYT4/290-329 | >E9KN92/34-55 | >U3DZ58/345-383 |
| >A0A2D0RZF6/294-333 | >E9KN93/34-55 | >U3EAL5/387-426 |
| >A0A2D4NGD5/97-130 | >E9KN94/34-55 | >U31844/347-386 |
| >A0A2F0B4V5/365-403 | >E9KN96/34-55 | >U3IZR9/371-410 |
| >A0A2F0BF71/1-26 | >E9KN98/34-55 | >U3JY64/391-430 |
| >A0A2F0BFK9/1-26 | >E9NME8/307-345 | >U3K4S7/359-398 |
| >A0A2G8L3N1/252-284 | >E9QG65/348-387 | >U6D8B7/305-343 |
| >A0A2G9RT86/18-57 | >F1MKA9/391-430 | >V4A869/356-396 |
| >A0A2I0LU30/391-430 | >F1NWD0/297-336 | >V8N2I9/1-18 |
| >A0A2I0LU33/293-332 | >F1P1U2/372-411 | >V8NA72/128-154 |
| >A0A2I0M380/275-314 | >F1PI27/309-347 | >V915U2/398-438 |
| >A0A2I0M383/297-336 | >F1PL57/391-430 | >W5KBK0/290-329 |
| >A0A2I0M3A5/297-336 | >F1PYN7/395-434 | >W5KI53/317-352 |
| >A0A2I0UNP3/391-430 | >F1QKD5/292-331 | >W5L3E6/353-392 |
| >A0A2I2U634/408-447 | >F1QU1/296-335 | >W5LNF4/309-348 |
| >A0A2I2UUR1/408-447 | >F1SY23/312-348 | >W5MCU3/391-430 |
| >A0A2I2Y444/391-430 | >F4YFP9/398-438 | >W5MCW2/294-333 |
| >A0A2I2Y5B0/391-430 | >F5A7P3/297-336 | >W5MD84/373-412 |
| >A0A2I2Y7Z8/280-318 | >F6MDM8/312-348 | >W5N8V4/315-352 |
| >A0A2I2YKP9/345-383 | >F6SSG7/372-406 | >W5N8V9/310-347 |
| >A0A2I2YQW0/208-247 | >F6TKT0/347-386 | >W5Q2R4/346-385 |
| >A0A2I2YW09/314-352 | >F6TL72/195-233 | >W5Q2R7/346-385 |
| >A0A2I2Z132/238-276 | >F6U7N6/202-241 | >W5QGZ8/386-425 |
| >A0A2I2Z434/391-430 | >F6VXD2/345-383 | >W5QGZ9/353-392 |
| >A0A2I2Z7P9/296-334 | >F6VXE7/242-280 | >W5U837/290-329 |
| >A0A212Z9N2/345-383 | >F6ZGN7/298-337 | >W5VJU6/268-305 |
| >A0A2I2ZDS0/375-414 | >F7A9M9/299-331 | >W8FSP6/309-347 |
| >A0A2I2Z1W0/288-326 | >F7A9U0/290-336 | >W8Q7P2/348-387 |
| >A0A2I2ZLA8/297-336 | >F7B2P6/352-391 | >W8Q8J6/385-424 |
| >A0A2I2ZMS7/345-383 | >F7DTE6/145-184 | >Z4YK94/344-382 |
| >A0A2I3GLU2/365-404 | >F7DUR2/389-428 | |

TABLE 14
Quad 68 & 69 Sequences

SEQ ID NO: 188 (Quad 68 nucleotide sequence)
ATGGGTTGGAGTTGTATAATTCTCTTCCTCGTCGCTACTGCTACTGGTGT
TCATTCTGAGGTCCAATTGTTGGAGTCCGGCGGCGGCGAGGTGCAACCAG
GTGGTTCACTCCGGTTGAGTTGCGCCGCGTCAGGCGGGATTTTCGCGATT
AAACCAATATCATGGTATAGGCAAGCGCCCGGGAAACAACGCGAATGGGT
GTCTACTACCACCAGTTCCGGGGCGACTAACTATGCGGAATCAGTAAAAG
GGCGCTTTACAATATCTCGCGATAATGCGAAGAATACTTTGTATTTGCAA
ATGTCATCTCTCAGGGCGGAAGACACTGCTGTTTATTATTGTAATGTCTT
TGAATACTGGGGTCAAGGTACGTTGGTGACTGTTAAGCCCGGTGGCAGTG
GCGGCTCAGAGGTTCAACTCCTTGAATCCGGAGGAGGTGAGGTCCAACCA
GGCGGAAGTCTCCGCCTTTCATGCGCAGCGTCCGGGTTTAGTTTCTCCAT
TAACGCAATGGGATGGTATCGCCAAGCACCGGGTAAAAGGCGCGAGTTCG
TTGCTGCTATTGAATCAGGTAGGAACACGGTTTACGCTGAATCCGTCAAA
GGGCGATTTACAATATCCCGTGATAATGCGAAGAATACAGTTTATTTGCA
AATGAGTTCACTCAGGGCAGAAGACACGGCGGTTTATTACTGTGGACTGC
TTAAAGGAAATCGGGTCGTCTCCCCCTCTGTCGCGTACTGGGGACAAGGA
ACCCTCGTGACCGTTAAACCCAAGAAGAAACCTCTCGATGGGGAGTACTT
CACTCTCCAAATTCGAGGTAGGGAGAGGTTTGAAATGTTTAGGGAACTCA
ATGAAGCTCTCGAGCTTAAAGACGCGCAAGCTGGTAAGGAACCAGGGCAT
CATCACCATCATCAT
SEQ ID NO: 189 (Quad 69 nucleotide sequence)
ATGGGATGGTCCTGTATTATACTCTTCTTGGTCGCAACCGCGACCGGGGT
ACATAGTGAGGTTCAACTCTTGGAGAGCGGCGGCGGCGAGGTCCAACCCG
GTGGTTCACTCAGGTTGTCCTGCGCAGCAAGTGGCGGAATTTTCGCGATT
AAACCTATTTCATGGTATAGGCAAGCTCCCGGGAAACAACGCGAATGGGT
CTCAACAACGACTTCATCTGGAGCTACAAACTATGCTGAATCAGTTAAAG
GTCGCTTTACAATTTCTCGTGATAATGCGAAGAATACTCTCTATCTGCAA
ATGTCATCATTGAGGGCTGAAGACACTGCTGTCTATTACTGTAATGTATT
TGAATACTGGGGACAAGGGACTCTCGTGACCGTTAAGCCCAAGAAGAAAC
CACTCGATGGAGAGTACTTCACTCTTCAAATACGCGGAAGGGAGCGGTTT
GAAATGTTTCGAGAATTGAATGAAGCGTTGGAGTTGAAAGACGCACAAGC
TGGCAAGGAACCAGGTGAGGTTCAATTGCTTGAGAGCGGCGGCGGGGAAG
TGCAACCAGGCGGTTCCCTCAGATTGAGCTGCGCTGCATCCGGATTTTCC
TTTTCAATTAACGCGATGGGTTGGTATCGACAAGCACCCGGGAAACGTCG
AGAGTTCGTTGCTGCGATAGAATCTGGAAGGAACACTGTTTACGCTGAAA
GTGTTAAAGGAAGGTTTACAATTTCCCGCGATAATGCAAAGAATACAGTC
TATCTTCAAATGTCCAGTCTTCGCGCGGAAGACACTGCAGTCTATTACTG
TGGTCTTCTCAAAGGGAATAGGGTTGTCTCCCCATCCGTCGCTTACTGGG
GACAAGGTACGCTCGTAACTGTCAAACCCCATCATCACCATCATCAT
SEQ ID NO: 190 (Quad 68 amino acid sequence)
MGWSCIILFLVATATGVHSEVQLLESGGGEVQPGGSLRLSCAASGGIFAI
KPISWYRQAPGKQREWVSTTTSSGATNYAESVKGRFTISRDNAKNTLYLQ
MSSLRAEDTAVYYCNVFEYWGQGTLVTVKPGGSGGSEVQLLESGGGEVQP
GGSLRLSCAASGFSFSINAMGWYRQAPGKRREFVAAIESGRNTVYAESVK
GRFTISRDNAKNTVYLQMSSLRAEDTAVYYCGLLKGNRVVSPSVAYWGQG
TLVTVKPKKKPLDGEYFTLQIRGRERFEMFRELNEALELKDAQAGKEPG
SEQ ID NO: 191 (Quad 69 amino acid sequence)
MGWSCIILFLVATATGVHSEVQLLESGGGEVQPGGSLRLSCAASGGIFAI
KPISWYRQAPGKQREWVSTITSSGATNYAESVKGRFTISRDNAKNTLYLQ
MSSLRAEDTAVYYCNVFEYWGQGTLVTVKPKKKPLDGEYFTLQIRGRERF
EMFRELNEALELKDAQAGKEPGEVQLLESGGGEVQPGGSLRLSCAASGFS
FSINAMGWYRQAPGKRREFVAAIESGRNTVYAESVKGRFTISRDNAKNTV
YLQMSSLRAEDTAVYYCGLLKGNRVVSPSVAYWGQGTLVTVKP

TABLE 15
A table summarizing EC₅₀ values of tetravalent and octavalent Quads (non-Ig
and Ig-like) anti-TNFα Quads to neutralize TNFα mediated cytotoxicity in WEHI cells.
The fold enhancement in potency for the different Quads over the monovalent control is
also shown

| | | | Fold enhancement |
|---|---|---|---|
| | | | of potency vs |
| Anti-TNFα dAb Quad | Valency | EC₅₀ (pM) | monovalent control |
| Anti-TNFα dAb (Monovalent Control) | 1 | 761.5 | — |
| Anti-TNFα dAb-TD | 4 | 96.6 | 8 |
| Anti-TNFα dAb-TD-dAb | 8 | 2.4 | 317 |
| Anti-TNFα dAb monomeric Ig-TD | 4 | 33.7 | 23 |
| Anti-TNFα dAb monomeric Ig-TD/dAb-Kappa | 8 | 2.8 | 272 |

TABLE 16
A table summarizing EC₅₀ values of non-Ig dodeca- and hexadeca-valent anti-
TNFα Quads to neutralize TNFα mediated cytotoxicity in WEHI cells. The fold
enhancement in potency for the different Quads over the monovalent control is also shown

| | | | Fold enhancement |
|---|---|---|---|
| | | | of potency vs |
| Anti-TNFα dAb Quad | Valency | EC₅₀ (pM) | monovalent control |
| Anti-TNFa dAb (Monovalent Control) | 1 | 1185 | — |
| Tandem anti-TNFα dAb-TD/anti-TNFα-Kappa | 12 | 1.824 | 650 |
| Tandem anti-TNFα dAb-TD/Tandem anti-TNFα-Kappa | 16 | 1.555 | 762 |
| Tandem anti-TNFα dAb-TD/anti-TNFα-Lambda | 12 | 2.041 | 581 |
| Tandem anti-TNFα dAb-TD/Tandem anti-TNFα-Lambda | 16 | 1.745 | 679 |

TABLE 17
DNA sequences encoding Quad polypeptides

| SEQ ID | QUAD NO./ | |
|---|---|---|
| NO. | NAME | NUCLEOTIDE SEQUENCE |
| 192 | Q92 | ATGGGCTGGTCATGTATAATCCTCTTTCTTGTAGCCACAGCTA |
| | | CCGGGGTACACTCAGAAGTTCAACTCGTCGAGAGTGGTGGCGG |
| | | CTTGGTCCAACCAGGTGGGAGTCTCCGCCTTAGCTGCGCAGCA |
| | | AGTGGGTTTACGTTTAGTGATTATTGGATGTATTGGGTGCGGC |
| | | AAGCTCCGGGAAAGGGACTGGAGTGGGTCTCTGAAATTAACAC |
| | | GAATGGTCTCATTACCAAATATCCTGATAGTGTAAAAGGACGC |
| | | TTCACAATTTCTAGGGATAATGCAAAGAACACTCTCTACCTTC |
| | | AAATGAATTCCCTGCGTCCCGAAGACACGGCGGTTTATTATTG |
| | | CGCTCGATCCCCTAGTGGGTTTAATCGAGGACAAGGAACCCTT |
| | | GTGACAGTCTCGAGTGCCTCCACTAAGGGGCCGAGTGTTTTTC |
| | | CACTTGCCCCATCCAGTAAGAGCACCTCTGGAGGAACTGCCGC |
| | | CCTGGGTTGCCTTGTTAAGGATTACTTCCCTGAGCCAGTAACT |
| | | GTTAGCTGGAACTCTGGCGCTCTGACCAGCGGAGTGCACACCT |
| | | TCCCTGCTGTGCTGCAGTCCTCAGGGCTGTACTCCCTTTCTAG |
| | | TGTCGTAACAGTGCCATCTTCTAGCCTGGGGACCCAGACGTAC |
| | | ATCTGTAACGTGAATCATAAACCCAGTAACACAAAGGTAGATA |
| | | AGAAGGTTGAACCTAAGTCCTGCGATAAGACACATACCGCCCC |
| | | TGAACTGCTGGGCGGACCTTCCGTGTTCCTGTTCCCCCCAAAG |
| | | CCCAAGGACACCCTGATGATCTCCCGGACCCCCGAAGTGACCT |
| | | GCGTGGTGGTGGATGTGTCCCACGAGGACCCTGAAGTGAAGTT |
| | | CAATTGGTACGTGGACGGCGTGGAAGTGCACAACGCCAAGACC |
| | | AAGCCTAGAGAGGAACAGTACAACTCCACCTACCGGGTGGTGT |
| | | CCGTGCTGACCGTGCTGCACCAGGATTGGCTGAACGGCAAAGA |
| | | GTACAAGTGCAAGGTGTCCAACAAGGCCCTGCCTGCCCCCATC |
| | | GAAAAGACCATCTCCAAGGCCAAGGGCCAGCCCCGGGAACCCC |
| | | AGGTGTACACACTGCCCCCTAGCAGGGACGAGCTGACCAAGAA |
| | | CCAGGTGTCCCTGACCTGTCTCGTGAAAGGCTTCTACCCCTCC |
| | | GATATCGCCGTGGAATGGGAGTCCAACGGCCAGCCTGAGAACA |
| | | ACTACAAGACCACCCCCCCTGTGCTGGACTCCGACGGCTCATT |
| | | CTTCCTGTACAGCAAGCTGACAGTGGACAAGTCCCGGTGGCAG |
| | | CAGGGCAACGTGTTCTCCTGCTCCGTGATGCACGAGGCCCTGC |
| | | ACAACCACTACACCCAGAAGTCCCTGTCCCTGAGCCCCGGCAA |
| | | GAAGAAAAAGCCCCTGGACGGCGAGTACTTCACACTGCAGATC |
| | | CGGGGCAGAGAACGCTTCGAGATGTTCAGAGAGCTGAACGAGG |
| | | CCCTGGAACTGAAGGATGCCCAGGCCGGAAAAGAGCCCGGC |
| 193 | Q93 | ATGGGCTGGTCATGTATAATCCTCTTTCTTGTAGCCACAGCTA |
| | | CCGGGGTACACTCAGAAGTTCAACTCGTCGAGAGTGGTGGCGG |
| | | CTTGGTCCAACCAGGTGGGAGTCTCCGCCTTAGCTGCGCAGCA |
| | | AGTGGGTTTACGTTTAGTGATTATTGGATGTATTGGGTGCGGC |
| | | AAGCTCCGGGAAAGGGACTGGAGTGGGTCTCTGAAATTAACAC |
| | | GAATGGTCTCATTACCAAATATCCTGATAGTGTAAAAGGACGC |
| | | TTCACAATTTCTAGGGATAATGCAAAGAACACTCTCTACCTTC |
| | | AAATGAATTCCCTGCGTCCCGAAGACACGGCGGTTTATTATTG |
| | | CGCTCGATCCCCTAGTGGGTTTAATCGAGGACAAGGAACCCTT |
| | | GTGACAGTAAGCTCAAAACGTACGGTGGCCGCTCCCTCCGTGT |
| | | TCATCTTCCCACCTTCCGACGAGCAGCTGAAGTCCGGCACCGC |
| | | TTCTGTCGTGTGCCTGCTGAACAACTTCTACCCCCGCGAGGCC |
| | | AAGGTGCAGTGGAAGGTGGACAACGCCCTGCAGTCCGGCAACT |
| | | CCCAGGAATCCGTGACCGAGCAGGACTCCAAGGACAGCACCTA |
| | | CTCCCTGTCCTCCACCCTGACCCTGTCCAAGGCCGACTACGAG |
| | | AAGCACAAGGTGTACGCCTGCGAAGTGACCCACCAGGGCCTGT |
| | | CTAGCCCCGTGACCAAGTCTTTCAACCGGGGCGAGTGT |
| 194 | Q113 | ATGGGTTGGTCTTGTATTATTCTTTTCCTCGTCGCAACCGCTA |
| | | CCGGGGTCCATTCCGAGGTCCAATTGCTTGAATCCGGAGGAGG |
| | | TGAAGTGCAACCCGGTGGGTCACTTCGGCTCTCCTGCGCCGCG |
| | | AGTGGCGGGATTTTCGCTATTAAACCAATTTCCTGGTATCGTC |
| | | AAGCACCAGGAAAACAACGAGAATGGGTGTCAACTACTACGTC |
| | | TTCTGGGGCAACTAACTATGCAGAATCAGTCAAAGGACGCTTT |
| | | ACGATTAGTCGAGATAATGCGAAGAATACTCTTTATCTCCAAA |
| | | TGTCATCACTCAGGGCAGAAGACACTGCTGTCTATTATTGTAA |
| | | TGTTTTCGAATACTGGGGTCAAGGAACTTTGGTGACCGTAAAG |
| | | CCCGGCGGCAGCGGCGGTAGTGAAGTCCAACTCCTCGAGAGTG |
| | | GAGGAGGGGAAGTGCAACCCGGCGGAAGTTTGCGGCTTAGTTG |
| | | CGCAGCTTCCGGTTTTAGCTTTAGTATAAACGCAATGGGATGG |
| | | TATCGCCAAGCTCCGGGGAAAAGGCGAGAGTTCGTAGCTGCAA |
| | | TTGAATCTGGACGTAACACGGTCTACGCAGAATCCGTCAAAGG |
| | | GCGTTTTACTATTAGTCGCGATAATGCTAAGAATACGGTTTAT |
| | | CTCCAAATGTCTTCACTCCGGGCAGAAGACACAGCTGTTTATT |
| | | ACTGTGGATTGCTCAAAGGAAATCGGGTCGTCTCACCGTCAGT |
| | | CGCGTACTGGGGACAAGGAACCCTTGTGACTGTTAAACCAGGT |
| | | GGTGGTGGGGACAAGACCCACACCGCCCCTGAACTGCTGGGCG |
| | | GACCTTCCGTGTTCCTGTTTCCTCCAAAGCCTAAGGACACCCT |
| | | GATGATCAGCAGAACCCCTGAAGTGACCTGCGTGGTGGTGGAT |
| | | GTGTCCCACGAGGATCCCGAAGTGAAGTTCAATTGGTACGTGG |
| | | ACGGCGTGGAAGTGCACAACGCCAAGACCAAGCCTAGAGAGGA |
| | | ACAGTACAACAGCACCTACAGAGTGGTGTCCGTGCTGACCGTG |
| | | CTGCACCAGGATTGGCTGAACGGCAAAGAGTACAAGTGCAAGG |
| | | TGTCCAACAAGGCCCTGCCTGCTCCTATCGAGAAAACCATCAG |
| | | CAAGGCCAAGGGCCAGCCTAGGGAACCCCAGGTTTACACACTG |
| | | CCTCCAAGCCGGGAAGAGATGACCAAGAACCAGGTGTCCCTGA |
| | | CCTGCCTCGTGAAGGGCTTCTACCCTTCCGATATCGCCGTGGA |
| | | ATGGGAGAGCAATGGCCAGCCAGAGAACAACTACAAGACAACC |
| | | CCTCCTGTGCTGGACAGCGACGGCTCATTCTTCCTGTACAGCA |
| | | AGCTGACAGTGGACAAGTCCAGATGGCAGCAGGGCAACGTGTT |
| | | CTCCTGCTCTGTGATGCACGAGGCCCTGCACAACCACTACACC |
| | | CAGAAGTCCCTGAGCCTGTCTCCTGGCAAAAAGAAAAAGCCCC |
| | | TGGACGGCGAGTACTTCACACTGCAAATCCGGGGCAGAGAACG |
| | | CTTCGAGATGTTCAGAGAGCTGAACGAGGCCCTGGAACTGAAG |
| | | GATGCCCAGGCCGGAAAAGAGCCCGGC |
| 195 | Q114 | ATGGGTTGGTCTTGTATTATTCTTTTCCTCGTCGCAACCGCTA |
| | | CCGGGGTCCATTCCGAGGTCCAATTGCTTGAATCCGGAGGAGG |
| | | TGAAGTGCAACCCGGTGGGTCACTTCGGCTCTCCTGCGCCGCG |
| | | AGTGGCGGGATTTTCGCTATTAAACCAATTTCCTGGTATCGTC |
| | | AAGCACCAGGAAAACAACGAGAATGGGTGTCAACTACTACGTC |
| | | TTCTGGGGCAACTAACTATGCAGAATCAGTCAAAGGACGCTTT |
| | | ACGATTAGTCGAGATAATGCGAAGAATACTCTTTATCTCCAAA |
| | | TGTCATCACTCAGGGCAGAAGACACTGCTGTCTATTATTGTAA |
| | | TGTTTTCGAATACTGGGGTCAAGGAACTTTGGTGACCGTAAAG |
| | | CCCGGCGGCAGCGGCGGTAGTGAAGTCCAACTCCTCGAGTCCG |
| | | GAGGAGGTGAAGTGCAACCCGGTGGGTCACTTCGGCTCTCCTG |
| | | CGCCGCGAGTGGCGGGATTTTCGCTATTAAACCAATTTCCTGG |
| | | TATCGTCAAGCACCAGGAAAACAACGAGAATGGGTGTCAACTA |
| | | CTACGTCTTCTGGGGCAACTAACTATGCAGAATCAGTCAAAGG |
| | | ACGCTTTACGATTAGTCGAGATAATGCGAAGAATACTCTTTAT |
| | | CTCCAAATGTCATCACTCAGGGCAGAAGACACTGCTGTCTATT |
| | | ATTGTAATGTTTTCGAATACTGGGGTCAAGGAACTTTGGTGAC |
| | | CGTAAAGCCCGGTGGTGGTGGGGACAAGACCCACACCGCCCCT |
| | | GAACTGCTGGGCGGACCTTCCGTGTTCCTGTTTCCTCCAAAGC |
| | | CTAAGGACACCCTGATGATCAGCAGAACCCCTGAAGTGACCTG |
| | | CGTGGTGGTGGATGTGTCCCACGAGGATCCCGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTC |
| | | CGTGCTGACCGTGCTGCACCAGGATTGGCTGAACGGCAAAGAG |
| | | TACAAGTGCAAGGTGTCCAACAAGGCCCTGCCTGCTCCTATCG |
| | | AGAAAACCATCAGCAAGGCCAAGGGCCAGCCTAGGGAACCCCA |
| | | GGTTTACACACTGCCTCCAAGCCGGGAAGAGATGACCAAGAAC |
| | | CAGGTGTCCCTGACCTGCCTCGTGAAGGGCTTCTACCCTTCCG |
| | | ATATCGCCGTGGAATGGGAGAGCAATGGCCAGCCAGAGAACAA |
| | | CTACAAGACAACCCCTCCTGTGCTGGACAGCGACGGCTCATTC |
| | | TTCCTGTACAGCAAGCTGACAGTGGACAAGTCCAGATGGCAGC |
| | | AGGGCAACGTGTTCTCCTGCTCTGTGATGCACGAGGCCCTGCA |
| | | CAACCACTACACCCAGAAGTCCCTGAGCCTGTCTCCTGGCAAA |
| | | AAGAAAAAGCCCCTGGACGGCGAGTACTTCACACTGCAAATCC |
| | | GGGGCAGAGAACGCTTCGAGATGTTCAGAGAGCTGAACGAGGC |
| | | CCTGGAACTGAAGGATGCCCAGGCCGGAAAAGAGCCCGGC |
| 196 | Q96 | ATGGGCTGGTCATGTATAATCCTCTTTCTTGTAGCCACAGCTA |
| | | CCGGGGTACACTCAAGCGATACCGGCAGACCCTTCGTGGAAAT |
| | | GTACAGCGAGATCCCCGAGATCATCCACATGACCGAGGGCAGA |
| | | GAGCTGGTCATCCCCTGCAGAGTGACAAGCCCCAACATCACCG |
| | | TGACTCTGAAGAAGTTCCCTCTGGACACACTGATCCCCGACGG |
| | | CAAGAGAATCATCTGGGACAGCCGGAAGGGCTTCATCATCAGC |
| | | AACGCCACCTACAAAGAGATCGGCCTGCTGACCTGTGAAGCCA |
| | | CCGTGAATGGCCACCTGTACAAGACCAACTACCTGACACACAG |
| | | ACAGACCAACACCATCATCGACGTGGTGCTGAGCCCTAGCCAC |
| | | GGCATTGAACTGTCTGTGGGCGAGAAGCTGGTGCTGAACTGTA |
| | | CCGCCAGAACCGAGCTGAACGTGGGCATCGACTTCAACTGGGA |
| | | GTACCCCAGCAGCAAGCACCAGCACAAGAAACTGGTCAACCGG |
| | | GACCTGAAAACCCAGAGCGGCAGCGAGATGAAGAAATTCCTGA |
| | | GCACCCTGACCATCGACGGCGTGACCAGATCTGACCAGGGCCT |
| | | GTACACATGTGCCGCCAGCTCTGGCCTGATGACCAAGAAAAAC |
| | | AGCACCTTCGTGCGGGTGCACGAGAAGGACAAGACCCACACCG |
| | | CCCCTGAACTGCTGGGCGGACCTTCCGTGTTCCTGTTTCCTCC |
| | | AAAGCCTAAGGACACCCTGATGATCAGCAGAACCCCTGAAGTG |
| | | ACCTGCGTGGTGGTGGATGTGTCCCACGAGGATCCCGAAGTGA |
| | | AGTTCAATTGGTACGTGGACGGCGTGGAAGTGCACAACGCCAA |
| | | GACCAAGCCTAGAGAGGAACAGTACAATAGCACCTACAGAGTG |
| | | GTGTCCGTGCTGACCGTGCTGCACCAGGATTGGCTGAACGGCA |
| | | AAGAGTACAAGTGCAAGGTGTCCAACAAGGCCCTGCCTGCTCC |
| | | TATCGAGAAAACCATCTCCAAGGCCAAGGGCCAGCCTAGGGAA |
| | | CCCCAGGTTTACACACTGCCTCCAAGCAGGGACGAGCTGACAA |
| | | AGAACCAGGTGTCCCTGACCTGCCTGGTCAAGGGCTTCTACCC |
| | | TTCCGATATCGCCGTGGAATGGGAGAGCAATGGCCAGCCTGAG |
| | | AACAACTACAAGACAACCCCTCCTGTGCTGGACAGCGACGGCT |
| | | CATTCTTCCTGTACAGCAAGCTGACAGTGGACAAGAGCAGATG |
| | | GCAGCAGGGCAACGTGTTCTCCTGCTCTGTGATGCACGAGGCC |
| | | CTGCACAACCACTACACCCAGAAGTCCCTGAGCCTGTCTCCTG |
| | | GAAAGAAAAAGCCCCTGGACGGCGAGTACTTCACACTGCAAAT |
| | | CCGGGGCAGAGAACGCTTCGAGATGTTCAGAGAGCTGAACGAG |
| | | GCCCTGGAACTGAAGGATGCCCAGGCCGGAAAAGAGCCCGGC |
| 197 | Q88 | ATGGGCTGGTCATGTATAATCCTCTTTCTTGTAGCCACAGCTA |
| | monovalent | CCGGGGTACACTCAGAAGTTCAACTCGTCGAGAGTGGTGGCGG |
| | | CTTGGTCCAACCAGGTGGGAGTCTCCGCCTTAGCTGCGCAGCA |
| | | AGTGGGTTTACGTTTAGTGATTATTGGATGTATTGGGTGCGGC |
| | | AAGCTCCGGGAAAGGGACTGGAGTGGGTCTCTGAAATTAACAC |
| | | GAATGGTCTCATTACCAAATATCCTGATAGTGTAAAAGGACGC |
| | | TTCACAATTTCTAGGGATAATGCAAAGAACACTCTCTACCTTC |
| | | AAATGAATTCCCTGCGTCCCGAAGACACGGCGGTTTATTATTG |
| | | CGCTCGATCCCCTAGTGGGTTTAATCGAGGACAAGGAACCCTT |
| | | GTGACAGTAAGCTCACAC |
| 198 | Q88 | ATGGGCTGGTCATGTATAATCCTCTTTCTTGTAGCCACAGCTA |
| | tetravalent | CCGGGGTACACTCAGAAGTTCAACTCGTCGAGAGTGGTGGCGG |
| | | CTTGGTCCAACCAGGTGGGAGTCTCCGCCTTAGCTGCGCAGCA |
| | | AGTGGGTTTACGTTTAGTGATTATTGGATGTATTGGGTGCGGC |
| | | AAGCTCCGGGAAAGGGACTGGAGTGGGTCTCTGAAATTAACAC |
| | | GAATGGTCTCATTACCAAATATCCTGATAGTGTAAAAGGACGC |
| | | TTCACAATTTCTAGGGATAATGCAAAGAACACTCTCTACCTTC |
| | | AAATGAATTCCCTGCGTCCCGAAGACACGGCGGTTTATTATTG |
| | | CGCTCGATCCCCTAGTGGGTTTAATCGAGGACAAGGAACCCTT |
| | | GTGACAGTAAGCTCAAAGAAGAAGCCCCTTGACGGCGAGTACT |
| | | TCACACTGCAGATCCGGGGCAGAGAACGCTTCGAGATGTTCAG |
| | | AGAGCTGAACGAGGCCCTGGAACTGAAGGATGCCCAGGCCGGA |
| | | AAAGAGCCCGGC |
| 199 | Q88 | ATGGGCTGGTCATGTATAATCCTCTTTCTTGTAGCCACAGCTA |
| | octavalent | CCGGGGTACACTCAGAAGTTCAACTCGTCGAGAGTGGTGGCGG |
| | | CTTGGTCCAACCAGGTGGGAGTCTCCGCCTTAGCTGCGCAGCA |
| | | AGTGGGTTTACGTTTAGTGATTATTGGATGTATTGGGTGCGGC |
| | | AAGCTCCGGGAAAGGGACTGGAGTGGGTCTCTGAAATTAACAC |
| | | GAATGGTCTCATTACCAAATATCCTGATAGTGTAAAAGGACGC |
| | | TTCACAATTTCTAGGGATAATGCAAAGAACACTCTCTACCTTC |
| | | AAATGAATTCCCTGCGTCCCGAAGACACGGCGGTTTATTATTG |
| | | CGCTCGATCCCCTAGTGGGTTTAATCGAGGACAAGGAACCCTT |
| | | GTGACAGTAAGCTCAAAGAAGAAGCCCCTTGACGGCGAGTACT |
| | | TTACTTTGCAAATACGAGGCAGAGAAAGATTTGAAATGTTTCG |
| | | GGAACTTAACGAAGCGCTGGAGCTGAAAGACGCGCAAGCCGGC |
| | | AAAGAACCCGGAGAAGTTCAACTCGTCGAGAGTGGTGGCGGCT |
| | | TGGTCCAACCAGGTGGGAGTCTCCGCCTTAGCTGCGCAGCAAG |
| | | TGGGTTTACGTTTAGTGATTATTGGATGTATTGGGTGCGGCAA |
| | | GCTCCGGGAAAGGGACTGGAGTGGGTCTCTGAAATTAACACGA |
| | | ATGGTCTCATTACCAAATATCCTGATAGTGTAAAAGGACGCTT |
| | | CACAATTTCTAGGGATAATGCAAAGAACACTCTCTACCTTCAA |
| | | ATGAATTCCCTGCGTCCCGAAGACACGGCGGTTTATTATTGCG |
| | | CTCGATCCCCTAGTGGGTTTAATCGAGGACAAGGAACCCTTGT |
| | | GACAGTAAGCTCACAC |
| 200 | Q142 | ATGGGCTGGTCATGTATAATCCTCTTTCTTGTAGCCACAGCTA |
| | | CCGGGGTACACTCAGAAGTTCAACTCGTCGAGAGTGGTGGCGG |
| | | CTTGGTCCAACCAGGTGGGAGTCTCCGCCTTAGCTGCGCAGCA |
| | | AGTGGGTTTACGTTTAGTGATTATTGGATGTATTGGGTGCGGC |
| | | AAGCTCCGGGAAAGGGACTGGAGTGGGTCTCTGAAATTAACAC |
| | | GAATGGTCTCATTACCAAATATCCTGATAGTGTAAAAGGACGC |
| | | TTCACAATTTCTAGGGATAATGCAAAGAACACTCTCTACCTTC |
| | | AAATGAATTCCCTGCGTCCCGAAGACACGGCGGTTTATTATTG |
| | | CGCTCGATCCCCTAGTGGGTTTAATCGAGGACAAGGAACCCTT |
| | | GTGACAGTCTCGAGTGGCGGCAGCGGCGGTAGTGAAGTTCAAC |
| | | TCGTCGAGAGTGGTGGCGGCTTGGTCCAACCAGGTGGGAGTCT |
| | | CCGCCTTAGCTGCGCAGCAAGTGGGTTTACGTTTAGTGATTAT |
| | | TGGATGTATTGGGTGCGGCAAGCTCCGGGAAAGGGACTGGAGT |
| | | GGGTCTCTGAAATTAACACGAATGGTCTCATTACCAAATATCC |
| | | TGATAGTGTAAAAGGACGCTTCACAATTTCTAGGGATAATGCA |
| | | AAGAACACTCTCTACCTTCAAATGAATTCCCTGCGTCCCGAAG |
| | | ACACGGCGGTTTATTATTGCGCTCGATCCCCTAGTGGGTTTAA |
| | | TCGAGGACAAGGAACCCTTGTGACAGTCTCGTCAGGCGGAGGC |
| | | GGTTCCGGAGGGGGAGGATCCGCCTCCACTAAGGGGCCGAGTG |
| | | TTTTTCCACTTGCCCCATCCAGTAAGAGCACCTCTGGAGGAAC |
| | | TGCCGCCCTGGGTTGCCTTGTTAAGGATTACTTCCCTGAGCCA |
| | | GTAACTGTTAGCTGGAACTCTGGCGCTCTGACCAGCGGAGTGC |
| | | ACACCTTCCCTGCTGTGCTGCAGTCCTCAGGGCTGTACTCCCT |
| | | TTCTAGTGTCGTAACAGTGCCATCTTCTAGCCTGGGGACCCAG |
| | | ACGTACATCTGTAACGTGAATCATAAACCCAGTAACACAAAGG |
| | | TAGATAAGAAGGTTGAACCTAAGTCCTGCGATAAGACACATAC |
| | | CAAGAAGAAACCACTGGATGGAGAATATTTCACCCTTCAGATC |
| | | CGTGGGCGTGAGCGCTTCGAGATGTTCCGAGAGCTGAATGAGG |
| | | CCTTGGAACTCAAGGATGCCCAGGCTGGGAAGGAGCCAGGGCA |
| | | C |
| 201 | Q135 | ATGGGCTGGTCATGTATAATCCTCTTTCTTGTAGCCACAGCTA |
| | | CCGGGGTACACTCAGAAGTTCAACTCGTCGAGAGTGGTGGCGG |
| | | CTTGGTCCAACCAGGTGGGAGTCTCCGCCTTAGCTGCGCAGCA |
| | | AGTGGGTTTACGTTTAGTGATTATTGGATGTATTGGGTGCGGC |
| | | AAGCTCCGGGAAAGGGACTGGAGTGGGTCTCTGAAATTAACAC |
| | | GAATGGTCTCATTACCAAATATCCTGATAGTGTAAAAGGACGC |
| | | TTCACAATTTCTAGGGATAATGCAAAGAACACTCTCTACCTTC |
| | | AAATGAATTCCCTGCGTCCCGAAGACACGGCGGTTTATTATTG |
| | | CGCTCGATCCCCTAGTGGGTTTAATCGAGGACAAGGAACCCTT |
| | | GTGACAGTCTCGAGTGGCGGAGGCGGTTCCGGAGGGGGAGGAT |
| | | CCGGACAGCCAAAAGCAGCCCCATCCGTAACTCTGTTCCCACC |
| | | TAGTTCAGAGGAGCTTCAAGCAAACAAAGCCACACTTGTTTGC |
| | | CTTATTAGTGATTTTTATCCCGGTGCCGTGACAGTTGCCTGGA |
| | | AAGCTGATAGCTCACCAGTGAAAGCTGGCGTGGAGACAACCAC |
| | | ACCATCTAAACAAAGCAATAACAAGTATGCTGCCAGCTCATAT |
| | | CTGAGTCTCACTCCAGAACAATGGAAGTCTCATCGGTCCTATA |
| | | GCTGTCAAGTGACCCACGAAGGCAGTACCGTCGAGAAGACCGT |
| | | GGCACCAACAGAGTGTAGC |
| 202 | Q136 | ATGGGCTGGTCATGTATAATCCTCTTTCTTGTAGCCACAGCTA |
| | | CCGGGGTACACTCAGAAGTTCAACTCGTCGAGAGTGGTGGCGG |
| | | CTTGGTCCAACCAGGTGGGAGTCTCCGCCTTAGCTGCGCAGCA |
| | | AGTGGGTTTACGTTTAGTGATTATTGGATGTATTGGGTGCGGC |
| | | AAGCTCCGGGAAAGGGACTGGAGTGGGTCTCTGAAATTAACAC |
| | | GAATGGTCTCATTACCAAATATCCTGATAGTGTAAAAGGACGC |
| | | TTCACAATTTCTAGGGATAATGCAAAGAACACTCTCTACCTTC |
| | | AAATGAATTCCCTGCGTCCCGAAGACACGGCGGTTTATTATTG |
| | | CGCTCGATCCCCTAGTGGGTTTAATCGAGGACAAGGAACCCTT |
| | | GTGACAGTCTCGAGTGGCGGAGGCGGTTCCGGAGGGGGAGGAT |
| | | CCAAACGTACGGTGGCCGCTCCCTCCGTGTTCATCTTCCCACC |
| | | TTCCGACGAGCAGCTGAAGTCCGGCACCGCTTCTGTCGTGTGC |
| | | CTGCTGAACAACTTCTACCCCCGCGAGGCCAAGGTGCAGTGGA |
| | | AGGTGGACAACGCCCTGCAATCCGGCAACTCCCAGGAATCCGT |
| | | GACCGAGCAGGACTCCAAGGACAGCACCTACTCCCTGTCCTCC |
| | | ACCCTGACCCTGTCCAAGGCCGACTACGAGAAGCACAAGGTGT |
| | | ACGCCTGCGAAGTGACCCACCAGGGCCTGTCTAGCCCCGTGAC |
| | | CAAGTCTTTCAACCGGGGCGAGTGT |
| 203 | Q144 | ATGGGCTGGTCATGTATAATCCTCTTTCTTGTAGCCACAGCTA |
| | | CCGGGGTACACTCAGAAGTTCAACTCGTCGAGAGTGGTGGCGG |
| | | CTTGGTCCAACCAGGTGGGAGTCTCCGCCTTAGCTGCGCAGCA |
| | | AGTGGGTTTACGTTTAGTGATTATTGGATGTATTGGGTGCGGC |
| | | AAGCTCCGGGAAAGGGACTGGAGTGGGTCTCTGAAATTAACAC |
| | | GAATGGTCTCATTACCAAATATCCTGATAGTGTAAAAGGACGC |
| | | TTCACAATTTCTAGGGATAATGCAAAGAACACTCTCTACCTTC |
| | | AAATGAATTCCCTGCGTCCCGAAGACACGGCGGTTTATTATTG |
| | | CGCTCGATCCCCTAGTGGGTTTAATCGAGGACAAGGAACCCTT |
| | | GTGACAGTCTCGAGTGGCGGCAGCGGCGGTAGTGAAGTTCAAC |
| | | TCGTCGAGAGTGGTGGCGGCTTGGTCCAACCAGGTGGGAGTCT |
| | | CCGCCTTAGCTGCGCAGCAAGTGGGTTTACGTTTAGTGATTAT |
| | | TGGATGTATTGGGTGCGGCAAGCTCCGGGAAAGGGACTGGAGT |
| | | GGGTCTCTGAAATTAACACGAATGGTCTCATTACCAAATATCC |
| | | TGATAGTGTAAAAGGACGCTTCACAATTTCTAGGGATAATGCA |
| | | AAGAACACTCTCTACCTTCAAATGAATTCCCTGCGTCCCGAAG |
| | | ACACGGCGGTTTATTATTGCGCTCGATCCCCTAGTGGGTTTAA |
| | | TCGAGGACAAGGAACCCTTGTGACAGTCTCGTCAGGCGGAGGC |
| | | GGTTCCGGAGGGGGAGGATCCGGACAGCCAAAAGCAGCCCCAT |
| | | CCGTAACTCTGTTCCCACCTAGTTCAGAGGAGCTTCAAGCAAA |
| | | CAAAGCCACACTTGTTTGCCTTATTAGTGATTTTTATCCCGGT |
| | | GCCGTGACAGTTGCCTGGAAAGCTGATAGCTCACCAGTGAAAG |
| | | CTGGCGTGGAGACAACCACACCATCTAAACAAAGCAATAACAA |
| | | GTATGCTGCCAGCTCATATCTGAGTCTCACTCCAGAACAATGG |
| | | AAGTCTCATCGGTCCTATAGCTGTCAAGTGACCCACGAAGGCA |
| | | GTACCGTCGAGAAGACCGTGGCACCAACAGAGTGTAGC |
| 204 | Q145 | ATGGGCTGGTCATGTATAATCCTCTTTCTTGTAGCCACAGCTA |
| | | CCGGGGTACACTCAGAAGTTCAACTCGTCGAGAGTGGTGGCGG |
| | | CTTGGTCCAACCAGGTGGGAGTCTCCGCCTTAGCTGCGCAGCA |
| | | AGTGGGTTTACGTTTAGTGATTATTGGATGTATTGGGTGCGGC |
| | | AAGCTCCGGGAAAGGGACTGGAGTGGGTCTCTGAAATTAACAC |
| | | GAATGGTCTCATTACCAAATATCCTGATAGTGTAAAAGGACGC |
| | | TTCACAATTTCTAGGGATAATGCAAAGAACACTCTCTACCTTC |
| | | AAATGAATTCCCTGCGTCCCGAAGACACGGCGGTTTATTATTG |
| | | CGCTCGATCCCCTAGTGGGTTTAATCGAGGACAAGGAACCCTT |
| | | GTGACAGTCTCGAGTGGCGGCAGCGGCGGTAGTGAAGTTCAAC |
| | | TCGTCGAGAGTGGTGGCGGCTTGGTCCAACCAGGTGGGAGTCT |
| | | CCGCCTTAGCTGCGCAGCAAGTGGGTTTACGTTTAGTGATTAT |
| | | TGGATGTATTGGGTGCGGCAAGCTCCGGGAAAGGGACTGGAGT |
| | | GGGTCTCTGAAATTAACACGAATGGTCTCATTACCAAATATCC |
| | | TGATAGTGTAAAAGGACGCTTCACAATTTCTAGGGATAATGCA |
| | | AAGAACACTCTCTACCTTCAAATGAATTCCCTGCGTCCCGAAG |
| | | ACACGGCGGTTTATTATTGCGCTCGATCCCCTAGTGGGTTTAA |
| | | TCGAGGACAAGGAACCCTTGTGACAGTCTCGTCAGGCGGAGGC |
| | | GGTTCCGGAGGGGGAGGATCCAAACGTACGGTGGCCGCTCCCT |
| | | CCGTGTTCATCTTCCCACCTTCCGACGAGCAGCTGAAGTCCGG |
| | | CACCGCTTCTGTCGTGTGCCTGCTGAACAACTTCTACCCCCGC |
| | | GAGGCCAAGGTGCAGTGGAAGGTGGACAACGCCCTGCAATCCG |
| | | GCAACTCCCAGGAATCCGTGACCGAGCAGGACTCCAAGGACAG |
| | | CACCTACTCCCTGTCCTCCACCCTGACCCTGTCCAAGGCCGAC |
| | | TACGAGAAGCACAAGGTGTACGCCTGCGAAGTGACCCACCAGG |
| | | GCCTGTCTAGCCCCGTGACCAAGTCTTTCAACCGGGGCGAGTG |
| | | T |
| 205 | Avelumab | ATGGGCTGGTCATGTATAATCCTCTTTCTTGTAGCCACAGCTA |
| | Fab | CCGGGGTACACTCAGAAGTGCAGCTGCTGGAATCTGGCGGAGG |
| | monomeric | ACTGGTTCAACCTGGCGGCTCTCTGAGACTGTCTTGTGCCGCC |
| | Ig Quad | AGCGGCTTCACCTTCAGCAGCTATATCATGATGTGGGTCCGAC |
| | (Heavy | AGGCCCCTGGCAAAGGCCTTGAATGGGTGTCCAGCATCTATCC |
| | Chain) | CAGCGGCGGCATCACCTTTTACGCCGACACAGTGAAGGGCAGA |
| | | TTCACCATCAGCCGGGACAACAGCAAGAACACCCTGTACCTGC |
| | | AGATGAACAGCCTGAGAGCCGAGGACACCGCCGTGTACTACTG |
| | | CGCCAGAATCAAGCTGGGCACCGTGACCACCGTGGATTATTGG |
| | | GGACAGGGCACCCTGGTCACCGTCTCGAGTGCCTCCACTAAGG |
| | | GGCCGAGTGTTTTTCCACTTGCCCCATCCAGTAAGAGCACCTC |
| | | TGGAGGAACTGCCGCCCTGGGTTGCCTTGTTAAGGATTACTTC |
| | | CCTGAGCCAGTAACTGTTAGCTGGAACTCTGGCGCTCTGACCA |
| | | GCGGAGTGCACACCTTCCCTGCTGTGCTGCAGTCCTCAGGGCT |
| | | GTACTCCCTTTCTAGTGTCGTAACAGTGCCATCTTCTAGCCTG |
| | | GGGACCCAGACGTACATCTGTAACGTGAATCATAAACCCAGTA |
| | | ACACAAAGGTAGATAAGAAGGTTGAACCTAAGTCCTGCGATAA |
| | | GACACATACCGCCCCTGAACTGCTGGGCGGACCTTCCGTGTTC |
| | | CTGTTCCCCCCAAAGCCCAAGGACACCCTGATGATCTCCCGGA |
| | | CCCCCGAAGTGACCTGCGTGGTGGTGGATGTGTCCCACGAGGA |
| | | CCCTGAAGTGAAGTTCAATTGGTACGTGGACGGCGTGGAAGTG |
| | | CACAACGCCAAGACCAAGCCTAGAGAGGAACAGTACAACTCCA |
| | | CCTACCGGGTGGTGTCCGTGCTGACCGTGCTGCACCAGGATTG |
| | | GCTGAACGGCAAAGAGTACAAGTGCAAGGTGTCCAACAAGGCC |
| | | CTGCCTGCCCCCATCGAAAAGACCATCTCCAAGGCCAAGGGCC |
| | | AGCCCCGGGAACCCCAGGTGTACACACTGCCCCCTAGCAGGGA |
| | | CGAGCTGACCAAGAACCAGGTGTCCCTGACCTGTCTCGTGAAA |
| | | GGCTTCTACCCCTCCGATATCGCCGTGGAATGGGAGTCCAACG |
| | | GCCAGCCTGAGAACAACTACAAGACCACCCCCCCTGTGCTGGA |
| | | CTCCGACGGCTCATTCTTCCTGTACAGCAAGCTGACAGTGGAC |
| | | AAGTCCCGGTGGCAGCAGGGCAACGTGTTCTCCTGCTCCGTGA |
| | | TGCACGAGGCCCTGCACAACCACTACACCCAGAAGTCCCTGTC |
| | | CCTGAGCCCCGGCAAGAAGAAAAAGCCCCTGGACGGCGAGTAC |
| | | TTCACACTGCAGATCCGGGGCAGAGAACGCTTCGAGATGTTCA |
| | | GAGAGCTGAACGAGGCCCTGGAACTGAAGGATGCCCAGGCCGG |
| | | AAAAGAGCCCGGC |
| 206 | Avelumab | ATGGGCTGGTCATGTATAATCCTCTTTCTTGTAGCCACAGCTA |
| | Light Chain | CCGGGGTACACTCACAGTCTGCTCTGACACAGCCTGCCTCTGT |
| | | GTCTGGCTCTCCTGGCCAGAGCATCACCATCAGCTGTACCGGC |
| | | ACCAGCTCTGATGTCGGCGGCTACAATTACGTGTCCTGGTATC |
| | | AGCAGCACCCCGGCAAGGCCCCTAAGCTGATGATCTACGACGT |
| | | GTCCAACAGACCCAGCGGCGTGTCCAATAGATTCTCCGGCAGC |
| | | AAGAGCGGCAACACCGCCAGCCTGACAATTAGCGGACTGCAGG |
| | | CCGAGGACGAGGCCGATTACTACTGTAGCAGCTACACCAGCTC |
| | | CAGCACCAGAGTGTTTGGCACCGGCACAAAAGTGACCGTGCTG |
| | | GGCCAGCCTAAGGCCAATCCTACCGTGACACTGTTCCCTCCAA |
| | | GCAGCGAGGAACTGCAGGCTAACAAGGCCACACTCGTGTGCCT |
| | | GATCAGCGACTTTTATCCTGGCGCCGTGACCGTGGCCTGGAAG |
| | | GCTGATGGATCTCCTGTGAAAGCCGGCGTGGAAACCACCAAGC |
| | | CTAGCAAGCAGAGCAACAACAAATACGCCGCCAGCAGCTACCT |
| | | GAGCCTGACACCTGAGCAGTGGAAGTCCCACAGATCCTACAGC |
| | | TGCCAAGTGACCCACGAGGGCAGCACCGTGGAAAAAACAGTGG |
| | | CCCCTACCGAGTGCAGC |
| 207 | Humira Fab | ATGGGATGGTCTTGTATAATTCTGTTCCTGGTGGCAACAGCAA |
| | monomeric | CAGGAGTGCATAGCGAGGTGCAGCTGGTGGAGTCTGGGGGAGG |
| | Ig Quad | CTTGGTACAGCCCGGCAGGTCCCTGAGACTCTCCTGTGCGGCC |
| | (Heavy | TCTGGATTCACCTTTGATGATTATGCCATGCACTGGGTCCGGC |
| | Chain) | AAGCTCCAGGGAAGGGCCTGGAATGGGTCTCAGCTATCACTTG |
| | | GAATAGTGGTCACATAGACTATGCGGACTCTGTGGAGGGCCGA |
| | | TTCACCATCTCCAGAGACAACGCCAAGAACTCCCTGTATCTGC |
| | | AAATGAACAGTCTGAGAGCTGAGGATACGGCCGTATATTACTG |
| | | TGCGAAAGTCTCGTACCTTAGCACCGCGTCCTCCCTTGACTAT |
| | | TGGGGCCAAGGTACCCTGGTCACCGTCTCGAGTGCCTCCACTA |
| | | AGGGGCCGAGTGTTTTTCCACTTGCCCCATCCAGTAAGAGCAC |
| | | CTCTGGAGGAACTGCCGCCCTGGGTTGCCTTGTTAAGGATTAC |
| | | TTCCCTGAGCCAGTAACTGTTAGCTGGAACTCTGGCGCTCTGA |
| | | CCAGCGGAGTGCACACCTTCCCTGCTGTGCTGCAGTCCTCAGG |
| | | GCTGTACTCCCTTTCTAGTGTCGTAACAGTGCCATCTTCTAGC |
| | | CTGGGGACCCAGACGTACATCTGTAACGTGAATCATAAACCCA |
| | | GTAACACAAAGGTAGATAAGAAGGTTGAACCTAAGTCCTGCGA |
| | | TAAGACACATACCGCCCCTGAACTGCTGGGCGGACCTTCCGTG |
| | | TTCCTGTTCCCCCCAAAGCCCAAGGACACCCTGATGATCTCCC |
| | | GGACCCCCGAAGTGACCTGCGTGGTGGTGGATGTGTCCCACGA |
| | | GGACCCTGAAGTGAAGTTCAATTGGTACGTGGACGGCGTGGAA |
| | | GTGCACAACGCCAAGACCAAGCCTAGAGAGGAACAGTACAACT |
| | | CCACCTACCGGGTGGTGTCCGTGCTGACCGTGCTGCACCAGGA |
| | | TTGGCTGAACGGCAAAGAGTACAAGTGCAAGGTGTCCAACAAG |
| | | GCCCTGCCTGCCCCCATCGAAAAGACCATCTCCAAGGCCAAGG |
| | | GCCAGCCCCGGGAACCCCAGGTGTACACACTGCCCCCTAGCAG |
| | | GGACGAGCTGACCAAGAACCAGGTGTCCCTGACCTGTCTCGTG |
| | | AAAGGCTTCTACCCCTCCGATATCGCCGTGGAATGGGAGTCCA |
| | | ACGGCCAGCCTGAGAACAACTACAAGACCACCCCCCCTGTGCT |
| | | GGACTCCGACGGCTCATTCTTCCTGTACAGCAAGCTGACAGTG |
| | | GACAAGTCCCGGTGGCAGCAGGGCAACGTGTTCTCCTGCTCCG |
| | | TGATGCACGAGGCCCTGCACAACCACTACACCCAGAAGTCCCT |
| | | GTCCCTGAGCCCCGGCAAGAAGAAAAAGCCCCTGGACGGCGAG |
| | | TACTTCACACTGCAGATCCGGGGCAGAGAACGCTTCGAGATGT |
| | | TCAGAGAGCTGAACGAGGCCCTGGAACTGAAGGATGCCCAGGC |
| | | CGGAAAAGAGCCCGGC |
| 208 | Humira | ATGGGATGGTCTTGTATAATTCTGTTCCTGGTGGCAACAGCAA |
| | Light Chain | CAGGAGTGCATAGCGACATCCAGATGACCCAGTCTCCATCCTC |
| | | CCTGTCTGCATCTGTAGGGGACAGAGTCACCATCACTTGTCGG |
| | | GCAAGTCAGGGCATCAGAAATTACTTAGCCTGGTATCAGCAAA |
| | | AACCAGGGAAAGCCCCTAAGCTCCTGATCTATGCTGCATCCAC |
| | | TTTGCAATCAGGGGTCCCATCTCGGTTCAGTGGCAGTGGATCT |
| | | GGGACAGATTTCACTCTCACCATCAGCAGCCTACAGCCTGAAG |
| | | ATGTTGCAACTTATTACTGTCAAAGGTATAACCGTGCACCGTA |
| | | TACTTTTGGCCAGGGGACCAAGGTGGAAATCAAACGTACGGTG |
| | | GCCGCTCCCTCCGTGTTCATCTTCCCACCTTCCGACGAGCAGC |
| | | TGAAGTCCGGCACCGCTTCTGTCGTGTGCCTGCTGAACAACTT |
| | | CTACCCCCGCGAGGCCAAGGTGCAGTGGAAGGTGGACAACGCC |
| | | CTGCAGTCCGGCAACTCCCAGGAATCCGTGACCGAGCAGGACT |
| | | CCAAGGACAGCACCTACTCCCTGTCCTCCACCCTGACCCTGTC |
| | | CAAGGCCGACTACGAGAAGCACAAGGTGTACGCCTGCGAAGTG |
| | | ACCCACCAGGGCCTGTCTAGCCCCGTGACCAAGTCTTTCAACC |
| | | GGGGCGAGTGT |

TABLE 18
Amino acid sequences of mature Quad polypeptides

| SEQ ID | QUAD NO./ | |
|---|---|---|
| NO. | NAME | AMINO ACID SEQUENCE |
| 209 | Q92 | EVQLVESGGGLVQPGGSLRLSCAASGFTFSDYWMYWVRQAPGK |
| | | GLEWVSEINTNGLITKYPDSVKGRFTISRDNAKNTLYLQMNSL |
| | | RPEDTAVYYCARSPSGFNRGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKKVEP |
| | | KSCDKTHTAPELLGGPSVFLFPPKPKDTLMISRTPEVICVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSRDELTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGKKKKPLDGEYFTLQIRGRERFEMFRELNEALELK |
| | | DAQAGKEPG |
| 210 | Q93 | EVQLVESGGGLVQPGGSLRLSCAASGFTFSDYWMYWVRQAPGK |
| | | GLEWVSEINTNGLITKYPDSVKGRFTISRDNAKNTLYLQMNSL |
| | | RPEDTAVYYCARSPSGFNRGQGTLVTVSSKRTVAAPSVFIFPP |
| | | SDEQLKSGTASVVCLLNNFYPREAKVQWKVDNALQSGNSQESV |
| | | TEQDSKDSTYSLSSTLTLSKADYEKHKVYACEVTHQGLSSPVT |
| | | KSFNRGEC |
| 211 | Q113 | EVQLLESGGGEVQPGGSLRLSCAASGGIFAIKPISWYRQAPGK |
| | | QREWVSTITSSGATNYAESVKGRFTISRDNAKNTLYLQMSSLR |
| | | AEDTAVYYCNVFEYWGQGTLVTVKPGGSGGSEVQLLESGGGEV |
| | | QPGGSLRLSCAASGFSFSINAMGWYRQAPGKRREFVAAIESGR |
| | | NTVYAESVKGRFTISRDNAKNTVYLQMSSLRAEDTAVYYCGLL |
| | | KGNRVVSPSVAYWGQGTLVTVKPGGGGDKTHTAPELLGGPSVF |
| | | LFPPKPKDTLMISRTPEVICVVVDVSHEDPEVKFNWYVDGVEV |
| | | HNAKTKPREEQYNSTYRVVSVLTVLHQDWLNGKEYKCKVSNKA |
| | | LPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGKKKKPLDGEY |
| | | FTLQIRGRERFEMFRELNEALELKDAQAGKEPG |
| 212 | Q114 | EVQLLESGGGEVQPGGSLRLSCAASGGIFAIKPISWYRQAPGK |
| | | QREWVSTITSSGATNYAESVKGRFTISRDNAKNTLYLQMSSLR |
| | | AEDTAVYYCNVFEYWGQGTLVTVKPGGSGGSEVQLLESGGGEV |
| | | QPGGSLRLSCAASGGIFAIKPISWYRQAPGKQREWVSTITSSG |
| | | ATNYAESVKGRFTISRDNAKNTLYLQMSSLRAEDTAVYYCNVF |
| | | EYWGQGTLVTVKPGGGGDKTHTAPELLGGPSVFLFPPKPKDTL |
| | | MISRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGNVF |
| | | SCSVMHEALHNHYTQKSLSLSPGKKKKPLDGEYFTLQIRGRER |
| | | FEMFRELNEALELKDAQAGKEPG |
| 213 | Q96 | SDTGRPFVEMYSEIPEIIHMTEGRELVIPCRVTSPNITVTLKK |
| | | FPLDTLIPDGKRIIWDSRKGFIISNATYKEIGLLTCEATVNGH |
| | | LYKTNYLTHRQTNTIIDVVLSPSHGIELSVGEKLVLNCTARTE |
| | | LNVGIDFNWEYPSSKHQHKKLVNRDLKTQSGSEMKKFLSILTI |
| | | DGVTRSDQGLYTCAASSGLMTKKNSTFVRVHEKDKTHTAPELL |
| | | GGPSVFLFPPKPKDTLMISRTPEVTCVVVDVSHEDPEVKFNWY |
| | | VDGVEVHNAKTKPREEQYNSTYRVVSVLTVLHQDWLNGKEYKC |
| | | KVSNKALPAPIEKTISKAKGQPREPQVYTLPPSRDELTKNQVS |
| | | LTCLVKGFYPSDIAVEWESNGQPENNYKTTPPVLDSDGSFFLY |
| | | SKLTVDKSRWQQGNVFSCSVMHEALHNHYTQKSLSLSPGKKKP |
| | | LDGEYFTLQIRGRERFEMFRELNEALELKDAQAGKEPG |
| 214 | Q88 | EVQLVESGGGLVQPGGSLRLSCAASGFTFSDYWMYWVRQAPGK |
| | monovalent | GLEWVSEINTNGLITKYPDSVKGRFTISRDNAKNTLYLQMNSL |
| | | RPEDTAVYYCARSPSGFNRGQGTLVTVSS |
| 215 | Q88 | EVQLVESGGGLVQPGGSLRLSCAASGFTFSDYWMYWVRQAPGK |
| | tetravalent | GLEWVSEINTNGLITKYPDSVKGRFTISRDNAKNTLYLQMNSL |
| | | RPEDTAVYYCARSPSGFNRGQGTLVTVSSKKKPLDGEYFTLQI |
| | | RGRERFEMFRELNEALELKDAQAGKEPG |
| 216 | Q88 | EVQLVESGGGLVQPGGSLRLSCAASGFTFSDYWMYWVRQAPGK |
| | octavalent | GLEWVSEINTNGLITKYPDSVKGRFTISRDNAKNTLYLQMNSL |
| | | RPEDTAVYYCARSPSGFNRGQGTLVTVSSKKKPLDGEYFTLQI |
| | | RGRERFEMFRELNEALELKDAQAGKEPGEVQLVESGGGLVQPG |
| | | GSLRLSCAASGFTFSDYWMYWVRQAPGKGLEWVSEINTNGLIT |
| | | KYPDSVKGRFTISRDNAKNTLYLQMNSLRPEDTAVYYCARSPS |
| | | GFNRGQGTLVTVSS |
| 217 | Q142 | EVQLVESGGGLVQPGGSLRLSCAASGFTFSDYWMYWVRQAPGK |
| | | GLEWVSEINTNGLITKYPDSVKGRFTISRDNAKNTLYLQMNSL |
| | | RPEDTAVYYCARSPSGFNRGQGTLVTVSSGGSGGSEVQLVESG |
| | | GGLVQPGGSLRLSCAASGFTFSDYWMYWVRQAPGKGLEWVSEI |
| | | NTNGLITKYPDSVKGRFTISRDNAKNTLYLQMNSLRPEDTAVY |
| | | YCARSPSGFNRGQGTLVTVSSGGGGSGGGGSASTKGPSVFPLA |
| | | PSSKSTSGGTAALGCLVKDYFPEPVTVSWNSGALTSGVHTFPA |
| | | VLQSSGLYSLSSVVTVPSSSLGTQTYICNVNHKPSNTKVDKKV |
| | | EPKSCDKTHTKKKPLDGEYFTLQIRGRERFEMFRELNEALELK |
| | | DAQAGKEPG |
| 218 | Q135 | EVQLVESGGGLVQPGGSLRLSCAASGFTFSDYWMYWVRQAPGK |
| | | GLEWVSEINTNGLITKYPDSVKGRFTISRDNAKNTLYLQMNSL |
| | | RPEDTAVYYCARSPSGFNRGQGTLVTVSSGGGGSGGGGSGQPK |
| | | AAPSVTLFPPSSEELQANKATLVCLISDFYPGAVTVAWKADSS |
| | | PVKAGVETTTPSKQSNNKYAASSYLSLTPEQWKSHRSYSCQVT |
| | | HEGSTVEKTVAPTECS |
| 219 | Q136 | EVQLVESGGGLVQPGGSLRLSCAASGFTFSDYWMYWVRQAPGK |
| | | GLEWVSEINTNGLITKYPDSVKGRFTISRDNAKNTLYLQMNSL |
| | | RPEDTAVYYCARSPSGFNRGQGTLVTVSSGGGGSGGGGSKRTV |
| | | AAPSVFIFPPSDEQLKSGTASVVCLLNNFYPREAKVQWKVDNA |
| | | LQSGNSQESVTEQDSKDSTYSLSSTLTLSKADYEKHKVYACEV |
| | | THQGLSSPVTKSFNRGEC |
| 220 | Q144 | EVQLVESGGGLVQPGGSLRLSCAASGFTFSDYWMYWVRQAPGK |
| | | GLEWVSEINTNGLITKYPDSVKGRFTISRDNAKNTLYLQMNSL |
| | | RPEDTAVYYCARSPSGFNRGQGTLVTVSSGGSGGSEVQLVESG |
| | | GGLVQPGGSLRLSCAASGFTFSDYWMYWVRQAPGKGLEWVSEI |
| | | NTNGLITKYPDSVKGRFTISRDNAKNTLYLQMNSLRPEDTAVY |
| | | YCARSPSGFNRGQGTLVTVSSGGGGSGGGGSGQPKAAPSVTLF |
| | | PPSSEELQANKATLVCLISDFYPGAVTVAWKADSSPVKAGVET |
| | | TTPSKQSNNKYAASSYLSLTPEQWKSHRSYSCQVTHEGSTVEK |
| | | TVAPTECS |
| 221 | Q145 | EVQLVESGGGLVQPGGSLRLSCAASGFTFSDYWMYWVRQAPGK |
| | | GLEWVSEINTNGLITKYPDSVKGRFTISRDNAKNTLYLQMNSL |
| | | RPEDTAVYYCARSPSGFNRGQGTLVTVSSGGSGGSEVQLVESG |
| | | GGLVQPGGSLRLSCAASGFTFSDYWMYWVRQAPGKGLEWVSEI |
| | | NTNGLITKYPDSVKGRFTISRDNAKNTLYLQMNSLRPEDTAVY |
| | | YCARSPSGFNRGQGTLVTVSSGGGGSGGGGSKRTVAAPSVFIF |
| | | PPSDEQLKSGTASVVCLLNNFYPREAKVQWKVDNALQSGNSQE |
| | | SVTEQDSKDSTYSLSSTLTLSKADYEKHKVYACEVTHQGLSSP |
| | | VTKSFNRGEC |
| 222 | Avelumab | EVQLLESGGGLVQPGGSLRLSCAASGFTFSSYIMMWVRQAPGK |
| | Fab | GLEWVSSIYPSGGITFYADTVKGRFTISRDNSKNTLYLQMNSL |
| | monomeric | RAEDTAVYYCARIKLGTVITVDYWGQGTLVTVSSASTKGPSVF |
| | Ig Quad | PLAPSSKSTSGGTAALGCLVKDYFPEPVTVSWNSGALTSGVHT |
| | (Heavy | FPAVLQSSGLYSLSSVVTVPSSSLGTQTYICNVNHKPSNTKVD |
| | Chain) | KKVEPKSCDKTHTAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSRDELTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPGKKKKPLDGEYFTLQIRGRERFEMFRELNE |
| | | ALELKDAQAGKEPG |
| 223 | Avelumab | QSALTQPASVSGSPGQSITISCTGTSSDVGGYNYVSWYQQHPG |
| | Light Chain | KAPKLMIYDVSNRPSGVSNRFSGSKSGNTASLTISGLQAEDEA |
| | | DYYCSSYTSSSTRVFGTGTKVTVLGQPKANPTVTLFPPSSEEL |
| | | QANKATLVCLISDFYPGAVTVAWKADGSPVKAGVETTKPSKQS |
| | | NNKYAASSYLSLTPEQWKSHRSYSCQVTHEGSTVEKTVAPTEC |
| | | S |
| 224 | Humira Fab | EVQLVESGGGLVQPGRSLRLSCAASGFTFDDYAMHWVRQAPGK |
| | monomeric | GLEWVSAITWNSGHIDYADSVEGRFTISRDNAKNSLYLQMNSL |
| | Ig Quad | RAEDTAVYYCAKVSYLSIASSLDYWGQGTLVTVSSASTKGPSV |
| | (Heavy | FPLAPSSKSTSGGTAALGCLVKDYFPEPVTVSWNSGALTSGVH |
| | Chain) | TFPAVLQSSGLYSLSSVVTVPSSSLGTQTYICNVNHKPSNTKV |
| | | DKKVEPKSCDKTHTAPELLGGPSVFLFPPKPKDTLMISRIPEV |
| | | TCVVVDVSHEDPEVKFNWYVDGVEVHNAKTKPREEQYNSTYRV |
| | | VSVLTVLHQDWLNGKEYKCKVSNKALPAPIEKTISKAKGQPRE |
| | | PQVYTLPPSRDELTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPGKKKKPLDGEYFTLQIRGRERFEMFRELN |
| | | EALELKDAQAGKEPG |
| 179 | Humira | DIQMTQSPSSLSASVGDRVTITCRASQGIRNYLAWYQQKPGKA |
| | Light Chain | PKLLIYAASTLQSGVPSRFSGSGSGTDFTLTISSLQPEDVATY |
| | | YCQRYNRAPYTFGQGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |

TABLE 19
List of viruses that can be bound by a multimer of the invention
Virus

Adeno-associated virus
Adenovirus
African swine fever virus
Autographa californica multiple nucleopolyhedrovirus
Barmah Forest virus (BFV)
Bombyx mor nucleopolyhedrovirus
Chickenpox
Chikungunya alphavirus
Chikungunya Virus Infection
COVID-19 (Coronavirus)
Coxsackieviruses
Cytomegalovirus (CMV)
Dengue virus (DENV)
Ebola
Enterovirus A & B
Epstein-Barr virus infection (EBV)
Hanta virus
Hantavirus Pulmonary Syndrome
Hepatitis virus (A, B, C, D or E)
Human herpesvirus 6 (HHV-6)
Human immunodeficiency virus (HIV)
Human papillomavirus
Human T- cell leukemia virus , type 1 (HTLV-1)
Infectious spleen and kidney necrosis virus
Influenza virus
Japanese encephalitis virus (JEV)
Kaposi's sarcoma-associated herpesvirus KSHV (HHV-8)
Mayaro virus (MAYV)
Measles
Merkel cell polyomavirus
Mumps
Norovirus
O'nyong-nyong virus (ONNV)
Orthmyxoviruses
Paramyxoviruses
Poliovirus
Rrabies
Respiratory syncytial virus
Retroviruses
Rhabdoviruses
Ross River virus (RRV)
Rubella
Severe acute respiratory syndrome (SARS)
Shingles
Simian immunodeficiency virus (SIV)
Simian varicella virus (SVV)
Simian Virus 40
Sindbis virus (SINV)
Singapore grouper iridovirus
Smallpox
Vaccinia virus
Varicella-zoster virus (VZV)
Vesicular stomatitis virus
West Nile virus (WNV)
Zika virus (ZIKV)

TABLE 20
List of example cell surface antigens on host cells or the surface of
viruses that could be specifically bound by a multimer of the invention,
eg, to block attachment onto and/or entry into host cells
Targets to block virus attachment/entry into host cells

CCR5
CCR5
CD21
CD4
CD46
CD80
CD86
Coxsackie-adenovirus receptor
CR2
CXCR4
DC-SIGN
DC-SIGNR
EGFR
G protein to cell surface receptors
GD1a glycan
Glycoproteins gB, gC, gD, gH and gL
Glycosaminoglycan
gp120
Hemagglutinin (HA1 and HA2)
Heparan sulphate
Heparan sulphate proteoglycans
Human mannose receptor C-type 1
Integrin α3β1
Integrins
Mannose-6-phosphate receptor
MHC1-α2
Myelin-associated glycoprotein
N-acetilneuraminic acid receptor
Nucleolin
Paired immunoglobulin-like receptor alpha
Phosphatidylserine
Proteoglycans
RIG-I
Sialic acid sugars and glycolipids
Surface heparan sulphate
TLR-2
TLR-3
TLR-7
TLR9
VCAM-1
xCT
αvβ3- and αvβ5-integrins
β1 and β3 integrins

1: A polypeptide comprising
(a) an antibody Fc region, wherein the Fc region comprises an antibody CH2 and an antibody CH3; and
(b) a self-associating multimerization domain (SAM);
wherein the CH2 comprises an antibody hinge sequence and is devoid of a core hinge region comprising amino acid sequence CXXC, wherein X is any amino acid.

2: The polypeptide of claim 1, wherein the CH2 is devoid of
a core hinge CXXC amino acid sequence, wherein each amino acid X is selected from a P, R and S.

3: The polypeptide of claim 1, wherein
(a) the CXXC sequence is selected from SEQ ID NOs: 180-182; or
(b) the CH2 is devoid of amino acid sequences SEQ ID NOs: 183-187.

4: The polypeptide of claim 1, wherein the CH2 comprises

| | (a) amino acid sequence |
|---|---|
| (SEQ ID NO: 163) | |
| | APELLGGPSV, |
| | or |
| (SEQ ID NO: 164) | |
| | PAPELLGGPSV; |
| | (b) amino acid sequence |
| (SEQ ID NO: 165) | |
| | APPVAGPSV, |
| | or |
| (SEQ ID NO: 166) | |
| | PAPPVAGPSV; |
| | (c) amino acid sequence |
| (SEQ ID NO: 175) | |
| | APEFLGGPSV, |
| | or |
| (SEQ ID NO: 176) | |
| | PAPEFLGGPSV; |
| | (d) amino acid sequence |
| (SEQ ID NO: 167) | |
| | EPKSCDKTHTPAPELLGGPSV |
| | or |
| (SEQ ID NO: 168) | |
| | EPKSCDKTHTAPELLGGPSV |
| | (e) amino acid sequence |
| (SEQ ID NO: 169) | |
| | ERKCCVEPAPPVAGPSV |
| | or |
| (SEQ ID NO: 170) | |
| | ERKCCVEAPPVAGPSV |
| | (f) amino acid sequence |
| (SEQ ID NO: 171) | |
| | ELKTPLGDTTHTPAPELLGGPSV |
| | or |
| (SEQ ID NO: 172) | |
| | ELKTPLGDTTHTPAPELLGGPSV |
| | (g) amino acid sequence |
| (SEQ ID NO: 173) | |
| | EPKSCDTPPPPAPELLGGPSV |
| | or |
| (SEQ ID NO: 174) | |
| | EPKSCDTPPPAPELLGGPSV |
| | (h) amino acid sequence |
| (SEQ ID NO: 177) | |
| | ESKYGPPPAPEFLGGPSV |
| | or |
| (SEQ ID NO: 178) | |
| | ESKYGPPAPEFLGGPSV |

5: The polypeptide of claim 1, wherein the CH2 and CH3 comprise
(a) human IgG1 CH2 and CH3 domains;
(b) human IgG2 CH2 and CH3 domains;
(c) human IgG3 CH2 and CH3 domains; or
(d) human IgG4 CH2 and CH3 domains.

6: The polypeptide of claim 1, wherein
(a) the CH2 domain comprises a human IgG1 CH2 domain and the core hinge region amino acid sequence is SEQ ID NO: 180;
(b) the CH2 domain comprises a human IgG2 CH2 domain and the core hinge region amino acid sequence is SEQ ID NO: 180;
(c) the CH2 domain comprises a human IgG3 CH2 domain and the core hinge region amino acid sequence is SEQ ID NO: 181; or
(d) the CH2 domain comprises a human IgG4 CH2 domain and the core hinge region amino acid sequence is SEQ ID NO: 182.

7: The polypeptide of claim 1, wherein the polypeptide comprises in N- to C-terminal direction the Fc region and the SAM, the Fc region comprising in N- to C-terminal direction the hinge sequence, a CH2 domain and a CH3 domain.

8: The polypeptide of claim 1, wherein the polypeptide comprises one or more epitope binding sites.

9: The polypeptide of claim 8, wherein the polypeptide comprises an antibody variable domain that is capable of specifically binding to a first epitope, wherein the variable domain is selected from an antibody single variable domain, a VH and a VL.

10: The polypeptide of claim 9, wherein the polypeptide comprises in N- to C-terminal direction
(a) the variable domain, the SAM and the Fc region;
(b) the Fc region, the SAM and the variable domain;
(c) the variable domain, the Fc region and the SAM;
(d) the SAM, the variable domain and the Fc region; or
(e) the SAM, the Fc region and the variable domain.

11: The polypeptide of claim 9, comprising a second antibody variable domain N- or C-terminal to the SAM, wherein the second variable domain is capable of specifically binding to a second epitope, wherein the first and second epitopes are identical or different.

12: The polypeptide of claim 1, wherein the SAM is a self-associating tetramerization domain (TD).

13: The polypeptide of claim 1, wherein
(i) the polypeptide comprises in N- to C-terminal direction:
A. a first antibody single variable domain (dAb), a linker and said SAM;
B. a first antibody single variable domain, a linker, said SAM and a second antibody single variable domain;
C. a first scFv, a linker and said SAM;
D. a first scFv, a linker, said SAM and a second scFv;
E. a first antibody single variable domain, said SAM and a first scFv;
F. a first scFv, a linker, said SAM and a first antibody single variable domain;
G. a first antibody variable domain, a first antibody constant domain and said SAM;
H. said SAM and a first antibody single variable domain;
I. said SAM and a first scFv;
J. said SAM, a first antibody constant domain and a first antibody variable domain; or
K. aid SAM, a first antibody variable domain and a first antibody constant domain;
or
(ii) the polypeptide comprises in N- to C-terminal direction:
A. a dAb and the SAM;
B. first dAb, the SAM and a second dAb;
C. a first scFv and the SAM;
D. a first scFv, the SAM and a second scFv;
E. a first scFv, the SAM and a first dAb;
F. first dAb, the Fc region and the SAM;
G. first scFv, the Fc region and the SAM;
H. a VH, a CH1, the Fc region and the SAM;
I. a VL, a CL, the Fc region and the SAM;
J. L dAb; the SAM and the Fc region;
K. scFv; the SAM and the Fc region;
L. a VH, a CH1, the SAM and the Fc region;
M. a VL, a CL, the SAM and the Fc region;
N. a first dAb, a second dAb and the SAM;
O. a VH, a CH1 and the SAM;
P. a VL, a CL and the SAM;
Q. a VH, a CH1, the SAM and a first dAb;
R. a VL, a CL, the SAM and a first dAb;
S. first dAb, a second dAb, the SAM and a third dAb;
T. first dAb, a second dAb, the SAM and a first scFv;
U. a first dAb, a second dAb, the SAM, a third dAb and a fourth dAb;
V. a first dAb, the Fc region, the SAM and a second dAb;
W. a first dAb, the Fc region, the SAM and a first scFv;
X. first dAb, a second dAb, the Fc region and the SAM;
Y. a first dAb, a second dAb, the Fc region, the SAM and a third dAb;
Z. a first dAb, a second dAb, the Fc region, the SAM and a first scFv; or
AA. a first dAb, a second dAb, the Fc region, the SAM, a third dAb and a fourth dAb;
or
(iii) the polypeptide comprises in C- to N-terminal direction:
A. a dAb and the SAM;
B. first dAb, the SAM and a second dAb;
C. a first scFv and the SAM;
D. a first scFv, the SAM and a second scFv;
E. a first scFv, the SAM and a first dAb;
F. first dAb, the Fc region and the SAM;
G. a first scFv, the Fc region and the SAM;
H. a VH, a CH1, the Fc region and the SAM;
I. a VL, a CL, the Fc region and the SAM;
J. a dAb; the SAM and the Fc region;
K. a scFv; the SAM and the Fc region;
L. a VH, a CH1, the SAM and the Fc region;
M. a VL, a CL, the SAM and the Fc region;
N. first dAb, a second dAb and the SAM;
O. VH, a CH1 and the SAM;
P. a VL, a CL and the SAM;
Q. a VH, a CH1, the SAM and a first dAb;
R. a VL, a CL, the SAM and a first dAb;
S. first dAb, a second dAb, the SAM and a third dAb;
T. a first dAb, a second dAb, the SAM and a first scFv;
U. a first dAb, a second dAb, the SAM, a third dAb and a fourth dAb;
V. a first dAb, the Fc region, the SAM and a second dAb;
W. first dAb, the Fc region, the SAM and a first scFv;
X. a first dAb, a second dAb, the Fc region and the SAM;
Y. a first dAb, a second dAb, the Fc region, the SAM and a third dAb;
Z. a first dAb, a second dAb, the Fc region, the SAM and a first scFv; or
AA. first dAb, a second dAb, the Fc region, the SAM, a third dAb and a fourth dAb.

14: A multimer of the polypeptide according to claim 1.

15: A multimer of a plurality of antibody Fc regions, wherein each Fc is comprised by a respective polypeptide, and wherein each Fc is unpaired with another Fc region.

16: A pharmaceutical composition comprising the polypeptide of claim 1.

17: A nucleic acid encoding Ie polypeptide of claim 1.

18: A method of binding multiple copies of an antigen, the method comprising combining the copies of the antigen with the multimer of claim 14, wherein the copies are bound by polypeptides of the multimer.

19: A method of treating or reducing the risk of a disease or condition in a human or animal subject, the method comprising administering the pharmaceutical composition of claim 16 to the subject, wherein multimers of the polypeptide in the composition specifically bind to a target antigen in the subject, wherein said binding mediates the treatment or reduction in risk of the disease or condition.

20: A method of producing a composition comprising a plurality of polypeptides, wherein each polypeptide comprises
(a) an antibody Fc region, wherein the Fc region comprises an antibody CH2 and an antibody CH3; and
(b) a self-associating multimerization domain (SAM):
and wherein the SAM is a self-associating tetramerization domain (TD), the method comprising providing eukaryotic host cells comprising the nucleic acid of claim 17, culturing the host cells, and allowing expression and secretion of tetramers of the polypeptides from the host cells.

21: The polypeptide of claim 6, wherein the CH2 domain comprises
(a) a human IgG1 CH2 domain, wherein the core hinge region amino acid sequence is SEQ ID NO: 180, and wherein the CH2 is devoid of upper hinge region amino acid sequence EPKSCDKTHT (SEQ ID NO: 183);
(b) a human IgG2 CH2 domain, wherein the core hinge region amino acid sequence is SEQ ID NO: 180, and wherein the CH2 is devoid of upper hinge region amino acid sequence ERKCCVE (SEQ ID NO: 184);
(c) a human IgG3 CH2 domain, wherein the core hinge region amino acid sequence is SEQ ID NO: 180, and wherein the CH2 is devoid of upper hinge region amino acid sequence ELKTPLGDTTHT (SEQ ID NO: 185) or upper hinge region amino acid sequence EPKSCDTPPP (SEQ ID NO: 186); or
(d) a human IgG4 CH2 domain, wherein the core hinge region amino acid sequence is SEQ ID NO: 180, and wherein the CH2 is devoid of upper hinge region amino acid sequence ESKYGPP (SEQ ID NO: 187).

22: The polypeptide of claim 12, wherein the tetramerization domain (TD)
(a) is a p53 TD or a homologue or orthologue thereof;
(b) is a NHR2 TD or a homologue or orthologue thereof; or
(c) comprises an amino acid sequence that is at least 80% identical to SEQ ID NO: 10 or 126.


SEQUENCE LISTING (240 sequences)

SEQ ID NO: 1 - protein, 393 aa, Homo sapiens [sequence omitted]

SEQ ID NO: 2 - protein, 341 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 3 - protein, 346 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 4 - protein, 354 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 5 - protein, 302 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 6 - protein, 307 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 7 - protein, 261 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 8 - protein, 261 aa, Homo sapiens; identical to SEQ ID NO: 7

SEQ ID NO: 9 - protein, 214 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 10 - protein, 32 aa, Homo sapiens
GEYFTLQIRGRERFEMFRELNEALELKDAQAG

SEQ ID NO: 11 - protein, 50 aa, Homo sapiens
RSPDDELLYLPVRGRETYEMLLKIKESLELMQYLPQHTIETYRQQQQQQH

SEQ ID NO: 12 - protein, 42 aa, Homo sapiens
KKRRHGDEDTYYLQVRGRENFEILMKLKESLELMELVPQPLV

SEQ ID NO: 13 - DNA, 1248 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 14 - DNA, 1263 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 15 - DNA, 1248 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 16 - DNA, 1263 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 17 - DNA, 954 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 18 - DNA, 969 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 19 - DNA, 999 bp, Artificial Sequence
  Synthetic Construct
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattacaccttggcagagagagcaaacaagtggaagacttaatgcctcgctggataaatcatcaggacgtagtactttatacattgcagcttctcagcctggtgactcagccacctacctctgtgctgtgaggcccctgcttgacggaacatacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 20 - DNA, 999 bp, Artificial Sequence
  Synthetic Construct
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattacaccttggcagagagagcaaacaagtggaagacttaatgcctcgctggataaatcatcaggacgtagtactttatacattgcagcttctcagcctggtgactcagccacctacctctgtgctgtgaggcccctgcttgacggaacatacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttccacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 21 - DNA, 681 bp, Artificial Sequence
  Synthetic Construct
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattacaccttggcagagagagcaaacaagtggaagacttaatgcctcgctggataaatcatcaggacgtagtactttatacattgcagcttctcagcctggtgactcagccacctacctctgtgctgtgaggcccctgcttgacggaacatacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcc

SEQ ID NO: 22 - DNA, 1647 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 23 - DNA, 1662 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 24 - DNA, 1293 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 25 - DNA, 1308 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 26 - DNA, 573 bp, Artificial Sequence
  Synthetic Construct
atggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 27 - DNA, 588 bp, Artificial Sequence
  Synthetic Construct
atggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaaggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 28 - DNA, 972 bp, Artificial Sequence
  Synthetic Construct
atggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 29 - DNA, 987 bp, Artificial Sequence
  Synthetic Construct
atggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaaggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 30 - DNA, 1182 bp, Artificial Sequence
  Synthetic Construct
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattacaccttggcagagagagcaaacaagtggaagacttaatgcctcgctggataaatcatcaggacgtagtactttatacattgcagcttctcagcctggtgactcagccacctacctctgtgctgtgaggcccctgcttgacggaacatacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 31 - DNA, 1206 bp, Artificial Sequence
  Synthetic Construct
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattacaccttggcagagagagcaaacaagtggaagacttaatgcctcgctggataaatcatcaggacgtagtactttatacattgcagcttctcagcctggtgactcagccacctacctctgtgctgtgaggcccctgcttgacggaacatacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaa

SEQ ID NO: 32 - DNA, 1104 bp, Artificial Sequence
  Synthetic Construct
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 33 - DNA, 885 bp, Artificial Sequence
  Synthetic Construct
atgaacttcgggctcagcttgattttccttgcccttattttaaaaggtgtccagtgtgaggtgcaactggtggagtctgggggaggcttagtgaagcctggggggtccctgaaactctcctgtgcagcctctggactcactttcagtagctatgccatgtcttgggttcgccagactccagagaagaggctggagtgggtcgcatccattagtagtggtggtttcacctactatccagacagtgtgaagggccgattcaccatctccagagataatgccaggaacatcctgtatctgcaaatgagcagtctgaggtctgaggacacggccatgtattactgtgcaagagacgaggtacgggggtacctcgatgtctggggcgcagggaccacggtcaccgtttcctcggccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 34 - DNA, 885 bp, Artificial Sequence; identical to SEQ ID NO: 33
  Synthetic Construct

SEQ ID NO: 35 - DNA, 717 bp, Artificial Sequence
  Synthetic Construct
atgaaatacctattgcctacggcagccgctggattgttattactcgcggcccagccggccatggcggcctacaaagatatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttattattgtcagcagtatagcaagcttccgtacacgttcggaggggggaccaagctggaaataaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 36 - DNA, 915 bp, Artificial Sequence
  Synthetic Construct
atgaacttcgggctcagcttgattttccttgcccttattttaaaaggtgtccagtgtgaggtgcaactggtggagtctgggggaggcttagtgaagcctggggggtccctgaaactctcctgtgcagcctctggactcactttcagtagctatgccatgtcttgggttcgccagactccagagaagaggctggagtgggtcgcatccattagtagtggtggtttcacctactatccagacagtgtgaagggccgattcaccatctccagagataatgccaggaacatcctgtatctgcaaatgagcagtctgaggtctgaggacacggccatgtattactgtgcaagagacgaggtacgggggtacctcgatgtctggggcgcagggaccacggtcaccgtttcctcggccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 37 - DNA, 930 bp, Artificial Sequence
  Synthetic Construct
atgaacttcgggctcagcttgattttccttgcccttattttaaaaggtgtccagtgtgaggtgcaactggtggagtctgggggaggcttagtgaagcctggggggtccctgaaactctcctgtgcagcctctggactcactttcagtagctatgccatgtcttgggttcgccagactccagagaagaggctggagtgggtcgcatccattagtagtggtggtttcacctactatccagacagtgtgaagggccgattcaccatctccagagataatgccaggaacatcctgtatctgcaaatgagcagtctgaggtctgaggacacggccatgtattactgtgcaagagacgaggtacgggggtacctcgatgtctggggcgcagggaccacggtcaccgtttcctcggccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 38 - DNA, 921 bp, Artificial Sequence
  Synthetic Construct
atgggatggtcttgtataattctgttcctggtggcaacagcaacaggagtgcatagcgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 39 - DNA, 936 bp, Artificial Sequence
  Synthetic Construct
atgggatggtcttgtataattctgttcctggtggcaacagcaacaggagtgcatagcgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 40 - DNA, 708 bp, Artificial Sequence
  Synthetic Construct
atggacatgagggtccctgctcagctcctgggactcctgctgctctggctcccagataccagatgtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 41 - DNA, 927 bp, Artificial Sequence
  Synthetic Construct
atgggatggtcttgtataattctgttcctggtggcaacagcaacaggagtgcatagcgaggtccaacttgtcgaaagtggcggcggtttggttcaacctggaggttcacttcgactgtcatgtgcagcgagcggttatacatttacgaattatggcatgaattgggttagacaggcaccaggaaagggactggagtgggtaggctggatcaatacctacacaggagaaccaacgtatgccgcagacttcaaacgacggtttacattttccttggatacctctaagtctacagcgtatctccaaatgaattcacttcgagcggaagataccgcggtctactattgcgccaaataccctcattattatgggtcatctcactggtatttcgatgtctggggtcagggaacactggtaaccgtgtcatccgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 42 - DNA, 942 bp, Artificial Sequence
  Synthetic Construct
atgggatggtcttgtataattctgttcctggtggcaacagcaacaggagtgcatagcgaggtccaacttgtcgaaagtggcggcggtttggttcaacctggaggttcacttcgactgtcatgtgcagcgagcggttatacatttacgaattatggcatgaattgggttagacaggcaccaggaaagggactggagtgggtaggctggatcaatacctacacaggagaaccaacgtatgccgcagacttcaaacgacggtttacattttccttggatacctctaagtctacagcgtatctccaaatgaattcacttcgagcggaagataccgcggtctactattgcgccaaataccctcattattatgggtcatctcactggtatttcgatgtctggggtcagggaacactggtaaccgtgtcatccgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 43 - DNA, 708 bp, Artificial Sequence
  Synthetic Construct
atggacatgagggtccctgctcagctcctggggctcctgcagctctggctctcaggtgccagatgtgacatccaaatgacccagagtccttccagcctcagtgcgtcagtgggagatcgagtgacgataacgtgttctgccagccaagacatttccaactatcttaattggtaccagcagaaaccgggaaaggccccgaaagtgctcatatactttaccagcagtcttcactctggagttcctagccggtttagcggctcaggtagtggcaccgatttcactctgaccattagttctcttcaaccggaagattttgcaacctactattgtcagcagtattcaacggtaccttggaccttcggccaaggcaccaaagtcgagattaagcgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 44 - DNA, 609 bp, Artificial Sequence
  Synthetic Construct
atggatatgcgcgtcccggcacagctgctcggcttgttgttgctgtggttgagagctaggtgcgatatacagatgactcagtccccttccagtctttcagccagtgtcggcgaccgggttaccattacttgtcgggcaagtcaatctatagatagttatttgcattggtatcaacaaaaaccaggcaaagcgcctaagttgttgatatattccgcatctgaactgcaatcaggcgttccttcacgcttttctggaagcggcagcggaaccgatttcactcttaccataagtagtctccagccggaggattttgctacatactattgtcaacaagtagtgtggcgaccgttcaccttcggacaggggacaaaagtagaaatcaagcggggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 45 - DNA, 633 bp, Artificial Sequence
  Synthetic Construct
atggagtttggcctcagttggttgtttttggtagcgaaaattaaagtacagtgtgaagtccaactcctggagagcggggggggtctggtacaaccaggcggctcactgcggcttagctgcgcagcctccgggttcacgttcgcacatgaaacgatggtgtgggtgcgccaggcaccggggaagggactcgaatgggtctcacatatacctcctgacggtcaggatcctttttacgcggactctgtgaagggacgattcacaataagtagagacaatagtaagaacaccctttatttgcagatgaacagtctgcgggcggaagatacagcagtatatcattgtgccctgctgcccaaacgaggtccgtggtttgactattggggacaggggactctcgttactgtaagctccggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 46 - DNA, 606 bp, Artificial Sequence
  Synthetic Construct
atggacatgagggtccccgctcagctcctggggctcctgctactctggctccgagccagatgtgatatacagatgacccaatcaccaagcagcttgtccgcttcagtgggcgacagggtaactataacatgccgcgcaagccaatggataggtccagaactctcatggtaccaacaaaaaccagggaaagcgccgaaactgcttatctatcacacaagcattttgcaatctggggtacctagtcgattcagtggctctggcagtgggactgactttacactcaccataagttctctccaaccagaggactttgcaacatactattgtcagcaatatatgtttcaaccacgcacctttggacaaggcacaaaagttgaaatccgcggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 47 - DNA, 609 bp, Artificial Sequence
  Synthetic Construct
atggacatgagggtccccgctcagctcctggggctcctgctactctggctccgagccagatgtgatattcaaatgacacagtcaccaacgagtctttccgcgagcgttggggaccgagtgacaataacttgtcgagcctctcagtggattggcaacttgctggactggtatcagcaaaagccgggagaagccccgaagctgctcatatactatgcttccttcctccagagtggagtacctagcagattcagcggggggggattcgggactgatttcactcttacaatcagctctcttcaacccgaggacttcgcaacgtactactgtcaacaagctaaccctgcgccgcttactttcggacaaggcactaaggtcgaaattaagcgaggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 48 - DNA, 954 bp, Artificial Sequence
  Synthetic Construct
atggacatgagggtccccgctcagctcctggggctcctgctactctggctccgagccagatgtgatatacagatgacccaatcaccaagcagcttgtccgcttcagtgggcgacagggtaactataacatgccgcgcaagccaatggataggtccagaactctcatggtaccaacaaaaaccagggaaagcgccgaaactgcttatctatcacacaagcattttgcaatctggggtacctagtcgattcagtggctctggcagtgggactgactttacactcaccataagttctctccaaccagaggactttgcaacatactattgtcagcaatatatgtttcaaccacgcacctttggacaaggcacaaaagttgaaatccgcggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaaggtggaggaggtagcgatattcaaatgacacagtcaccaacgagtctttccgcgagcgttggggaccgagtgacaataacttgtcgagcctctcagtggattggcaacttgctggactggtatcagcaaaagccgggagaagccccgaagctgctcatatactatgcttccttcctccagagtggagtacctagcagattcagcggggggggattcgggactgatttcactcttacaatcagctctcttcaacccgaggacttcgcaacgtactactgtcaacaagctaaccctgcgccgcttactttcggacaaggcactaaggtcgaaattaagcga

SEQ ID NO: 49 - DNA, 609 bp, Artificial Sequence
  Synthetic Construct
atggacatgagggtccccgctcagctcctggggctcctgctactctggctccgagccagatgtgacattcagatgactcagtcaccatcctcattgtctgcatcagttggtgaccgagttacgatcacatgtcgagcaagccaaaatatagattccagactttcatggtaccagcagaagcctggtaaagcgccgaaactcctcatatatcgcacgagcgtattgcaatctggtgtgccttctcgattttcaggatctgggtctggcactgacttcaccttgacaatatcttctcttcagcccgaagatttcgctacctactactgccaacaatgggacatgtttcctctgaccttcggacagggtacaaaggtcgagattaaacggggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 50 - DNA, 957 bp, Artificial Sequence
  Synthetic Construct
atggacatgagggtccccgctcagctcctggggctcctgctactctggctccgagccagatgtgacattcagatgactcagtcaccatcctcattgtctgcatcagttggtgaccgagttacgatcacatgtcgagcaagccaaaatatagattccagactttcatggtaccagcagaagcctggtaaagcgccgaaactcctcatatatcgcacgagcgtattgcaatctggtgtgccttctcgattttcaggatctgggtctggcactgacttcaccttgacaatatcttctcttcagcccgaagatttcgctacctactactgccaacaatgggacatgtttcctctgaccttcggacagggtacaaaggtcgagattaaacggggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaaggtggaggaggtagcgatatacagatgactcaatccccttcatccctctcagcttccgtaggggacagagttactataacgtgtcgagctagtcaagacataggtgatcgcctgaggtggtatcagcaaaaaccgggtaaagcacctaaactcctcatatatcatggttccaggttggagtcaggcgtgccgtcacgattctctgggtcacgctctggcactgacttcacattgacgattagttctctccagcccgaagacttcgccacctactactgtcaacagcaatggtttcgcccgtatacttttgggcagggtacaaaggttgagattaaacgg

SEQ ID NO: 51 - protein, 121 aa, Artificial Sequence [sequence omitted]

SEQ ID NO: 52 - protein, 108 aa, Artificial Sequence
  Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKR

SEQ ID NO: 53 - protein, 121 aa, Artificial Sequence [sequence omitted]

SEQ ID NO: 54 - protein, 107 aa, Artificial Sequence
  Synthetic Construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 55 - protein, 123 aa, Artificial Sequence [sequence omitted]

SEQ ID NO: 56 - protein, 108 aa, Artificial Sequence
  Synthetic Construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 57 - protein, 119 aa, Artificial Sequence [sequence omitted]

SEQ ID NO: 58 - protein, 108 aa, Artificial Sequence
  Synthetic Construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKR

SEQ ID NO: 59 - protein, 119 aa, Artificial Sequence
  Synthetic Construct
EVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSS

SEQ ID NO: 60 - protein, 108 aa, Artificial Sequence
  Synthetic Construct
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKR

SEQ ID NO: 61 - protein, 113 aa, Artificial Sequence [sequence omitted]

SEQ ID NO: 62 - protein, 108 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKR

SEQ ID NO: 63 - protein, 118 aa, Artificial Sequence
  Synthetic Construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 64 - protein, 109 aa, Artificial Sequence
  Synthetic Construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKR

SEQ ID NO: 65 - protein, 119 aa, Artificial Sequence
  Synthetic Construct
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSS

SEQ ID NO: 66 - protein, 108 aa, Artificial Sequence
  Synthetic Construct
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 67 - protein, 107 aa, Artificial Sequence
  Synthetic Construct
DIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIR

SEQ ID NO: 68 - protein, 108 aa, Artificial Sequence
  Synthetic Construct
DIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKR

SEQ ID NO: 69 - protein, 108 aa, Artificial Sequence
  Synthetic Construct
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGQGTKVEIKR

SEQ ID NO: 70 - protein, 108 aa, Artificial Sequence
  Synthetic Construct
DIQMTQSPSSLSASVGDRVTITCRASQDIGDRLRWYQQKPGKAPKLLIYHGSRLESGVPSRFSGSRSGTDFTLTISSLQPEDFATYYCQQQWFRPYTFGQGTKVEIKR

SEQ ID NO: 71 - protein, 115 aa, Artificial Sequence
  Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPY

SEQ ID NO: 72 - protein, 112 aa, Artificial Sequence
  Synthetic Construct
NAGVTQTPKFQVLKTGQSMTLQCAQDMNHEYMSWYRQDPGMGLRLIHYSVAIQTDQGEVPNGYNVSRSTIEDFPLRLLSAAPSQTSVYFCASSYLGNTGELFFGEGSRLTVL

SEQ ID NO: 73 - protein, 133 aa, Homo sapiens [sequence omitted]

SEQ ID NO: 74 - protein, 115 aa, Artificial Sequence [sequence omitted]

SEQ ID NO: 75 - protein, 108 aa, Artificial Sequence
  Synthetic Construct
DIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 76 - protein, 104 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDV

SEQ ID NO: 77 - protein, 101 aa, Artificial Sequence
  Synthetic Construct
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 78 - protein, 431 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 31 aa, Artificial Sequence
  Synthetic Construct
HAPGTFTSDVSSYLEGQAAKEFIAWLVKGRG

SEQ ID NO: 80 - protein, 34 aa, Artificial Sequence
  Synthetic Construct
IKPEAPGEDASPEELNRYYASLRHYLNLVTRQRY

SEQ ID NO: 81 - protein, 39 aa, Artificial Sequence
  Synthetic Construct
HGEGTFTSDLSKQMEEEAVRLFIEWLKNGGPSSGAPPPS

SEQ ID NO: 82 - protein, 275 aa, Artificial Sequence
  Synthetic Construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 83 - protein, 397 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLTDREWAEEWKHLDHLLNCIMDMVEKTRRSLTVLRRCQEADREELNYWIRRYSDAE

SEQ ID NO: 84 - protein, 402 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLTDREWAEEWKHLDHLLNCIMDMVEKTRRSLTVLRRCQEADREELNYWIRRYSDAE

SEQ ID NO: 85 - protein, 397 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLTDREWAEEWKHLDHLLNCIMDMVEKTRRSLTVLRRCQEADREELNYWIRRYSDAE

SEQ ID NO: 86 - protein, 402 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLTDREWAEEWKHLDHLLNCIMDMVEKTRRSLTVLRRCQEADREELNYWIRRYSDAE

SEQ ID NO: 87 - protein, 299 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADLTDREWAEEWKHLDHLLNCIMDMVEKTRRSLTVLRRCQEADREELNYWIRRYSDAE

SEQ ID NO: 88 - protein, 304 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGGGGSLTDREWAEEWKHLDHLLNCIMDMVEKTRRSLTVLRRCQEADREELNYWIRRYSDAE

SEQ ID NO: 89 - protein, 314 aa, Artificial Sequence
  Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 314 aa, Artificial Sequence
  Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 208 aa, Artificial Sequence
  Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 92 - protein, 530 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLTDREWAEEWKHLDHLLNCIMDMVEKTRRSLTVLRRCQEADREELNYWIRRYSDAEAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 93 - protein, 535 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLTDREWAEEWKHLDHLLNCIMDMVEKTRRSLTVLRRCQEADREELNYWIRRYSDAEAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 94 - protein, 412 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPLTDREWAEEWKHLDHLLNCIMDMVEKTRRSLTVLRRCQEADREELNYWIRRYSDAE

SEQ ID NO: 95 - protein, 417 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGGGGSLTDREWAEEWKHLDHLLNCIMDMVEKTRRSLTVLRRCQEADREELNYWIRRYSDAE

SEQ ID NO: 96 - protein, 172 aa, Artificial Sequence
  Synthetic Construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKLTDREWAEEWKHLDHLLNCIMDMVEKTRRSLTVLRRCQEADREELNYWIRRYSDAE

SEQ ID NO: 97 - protein, 177 aa, Artificial Sequence
  Synthetic Construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKGGGGSLTDREWAEEWKHLDHLLNCIMDMVEKTRRSLTVLRRCQEADREELNYWIRRYSDAE

SEQ ID NO: 98 - protein, 305 aa, Artificial Sequence
  Synthetic Construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKLTDREWAEEWKHLDHLLNCIMDMVEKTRRSLTVLRRCQEADREELNYWIRRYSDAEAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 99 - protein, 310 aa, Artificial Sequence
  Synthetic Construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKGGGGSLTDREWAEEWKHLDHLLNCIMDMVEKTRRSLTVLRRCQEADREELNYWIRRYSDAEAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 100 - protein, 378 aa, Artificial Sequence
  Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLHGTRQEEMIDHRLTDREWAEEWKHLDHLLNCIMDMVEKTRRSLTVLRRCQEADREELNYWIRRYSDAEDLK

SEQ ID NO: 101 - protein, 383 aa, Artificial Sequence
  Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLHGTRQEEMIDHRLTDREWAEEWKHLDHLLNCIMDMVEKTRRSLTVLRRCQEADREELNYWIRRYSDAEDLK

SEQ ID NO: 102 - protein, 349 aa, Artificial Sequence
  Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 291 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLHGTRQEEMIDHRLTDREWAEEWKHLDHLLNCIMDMVEKTRRSLTVLRRCQEADREELNYWIRRYSDAEDLK

SEQ ID NO: 104 - protein, 296 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLHGTRQEEMIDHRLTDREWAEEWKHLDHLLNCIMDMVEKTRRSLTVLRRCQEADREELNYWIRRYSDAEDLK

SEQ ID NO: 105 - protein, 214 aa, Artificial Sequence
  Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 293 aa, Artificial Sequence
  Synthetic Construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLHGTRQEEMIDHRLTDREWAEEWKHLDHLLNCIMDMVEKTRRSLTVLRRCQEADREELNYWIRRYSDAEDLK

SEQ ID NO: 107 - protein, 298 aa, Artificial Sequence
  Synthetic Construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLHGTRQEEMIDHRLTDREWAEEWKHLDHLLNCIMDMVEKTRRSLTVLRRCQEADREELNYWIRRYSDAEDLK

SEQ ID NO: 108 - protein, 214 aa, Artificial Sequence
  Synthetic Construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 185 aa, Artificial Sequence
  Synthetic Construct
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKRGGGGSLHGTRQEEMIDHRLTDREWAEEWKHLDHLLNCIMDMVEKTRRSLTVLRRCQEADREELNYWIRRYSDAEDLK

SEQ ID NO: 110 - protein, 196 aa, Artificial Sequence
  Synthetic Construct
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSSGGGGSLHGTRQEEMIDHRLTDREWAEEWKHLDHLLNCIMDMVEKTRRSLTVLRRCQEADREELNYWIRRYSDAEDLK

SEQ ID NO: 111 - protein, 184 aa, Artificial Sequence
  Synthetic Construct
DIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIRGGGGSLHGTRQEEMIDHRLTDREWAEEWKHLDHLLNCIMDMVEKTRRSLTVLRRCQEADREELNYWIRRYSDAEDLK

SEQ ID NO: 112 - protein, 185 aa, Artificial Sequence
  Synthetic Construct
DIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKRGGGGSLHGTRQEEMIDHRLTDREWAEEWKHLDHLLNCIMDMVEKTRRSLTVLRRCQEADREELNYWIRRYSDAEDLK

SEQ ID NO: 113 - protein, 297 aa, Artificial Sequence
  Synthetic Construct
DIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIRGGGGSLHGTRQEEMIDHRLTDREWAEEWKHLDHLLNCIMDMVEKTRRSLTVLRRCQEADREELNYWIRRYSDAEDLKGGGGSDIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKR

SEQ ID NO: 114 - protein, 185 aa, Artificial Sequence
  Synthetic Construct
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGQGTKVEIKRGGGGSLHGTRQEEMIDHRLTDREWAEEWKHLDHLLNCIMDMVEKTRRSLTVLRRCQEADREELNYWIRRYSDAEDLK

SEQ ID NO: 115 - protein, 298 aa, Artificial Sequence
  Synthetic Construct
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGQGTKVEIKRGGGGSLHGTRQEEMIDHRLTDREWAEEWKHLDHLLNCIMDMVEKTRRSLTVLRRCQEADREELNYWIRRYSDAEDLKGGGGSDIQMTQSPSSLSASVGDRVTITCRASQDIGDRLRWYQQKPGKAPKLLIYHGSRLESGVPSRFSGSRSGTDFTLTISSLQPEDFATYYCQQQWFRPYTFGQGTKVEIKR

SEQ ID NO: 116 - DNA, 168 bp, Artificial Sequence
  Synthetic Construct
ctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 117 - DNA, 207 bp, Artificial Sequence
  Synthetic Construct
ttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 118 - DNA, 201 bp, Artificial Sequence
  Synthetic Construct
ggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 119 - DNA, 141 bp, Artificial Sequence
  Synthetic Construct
caagaagaaatgattgatcacagactaacagacagagaatgggcagaagagtggaaacatcttgaccatctgttaaactgcataatggacatggtagaaaaaacaaggcgatctctcaccgtactaaggcggtgtcaagaa

SEQ ID NO: 120 - protein, 56 aa, Artificial Sequence
  Synthetic Construct
LTDREWAEEWKHLDHLLNCIMDMVEKTRRSLTVLRRCQEADREELNYWIRRYSDAE

SEQ ID NO: 121 - protein, 72 aa, Artificial Sequence
  Synthetic Construct
LHGTRQEEMIDHRLTDREWAEEWKHLDHLLNCIMDMVEKTRRSLTVLRRCQEADREELNYWIRRYSDAEDLK

SEQ ID NO: 122 - protein, 67 aa, Artificial Sequence
  Synthetic Construct
GTRQEEMIDHRLTDREWAEEWKHLDHLLNCIMDMVEKTRRSLTVLRRCQEADREELNYWIRRYSDAE

SEQ ID NO: 123 - protein, 47 aa, Artificial Sequence
  Synthetic Construct
QEEMIDHRLTDREWAEEWKHLDHLLNCIMDMVEKTRRSLTVLRRCQE

SEQ ID NO: 124 - DNA, 126 bp, Artificial Sequence
  Synthetic Construct
aagaagaaaccactggatggagaatatttcacccttcagatccgtgggcgtgagcgcttcgagatgttccgagagctgaatgaggccttggaactcaaggatgcccaggctgggaaggagccaggg

SEQ ID NO: 125 - DNA, 96 bp, Artificial Sequence
  Synthetic Construct
ggagaatatttcacccttcagatccgtgggcgtgagcgcttcgagatgttccgagagctgaatgaggccttggaactcaaggatgcccaggctggg

SEQ ID NO: 126 - protein, 42 aa, Artificial Sequence
  Synthetic Construct
KKKPLDGEYFTLQIRGRERFEMFRELNEALELKDAQAGKEPG

SEQ ID NO: 127 - DNA, 593 bp, Artificial Sequence
  Synthetic Construct
cacgtgatggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaaaagaagaaaccactggatggagaatatttcacccttcagatccgtgggcgtgagcgcttcgagatgttccgagagctgaatgaggccttggaactcaaggatgcccaggctgggaaggagccaggggactacaaggacgacgacgacaagcaccaccaccatcaccactgataagcggccgc

SEQ ID NO: 128 - protein, 197 aa, Artificial Sequence
  Synthetic Construct
  VARIANT (194)..(195): Xaa = any amino acid
HVMELGLSWVVLAALLQGVQAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKKKKPLDGEYFTLQIRGRERFEMFRELNEALELKDAQAGKEPGDYKDDDDKHHHHHHXXAA

SEQ ID NO: 129 - DNA, 608 bp, Artificial Sequence
  Synthetic Construct
cacgtgatggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaaggaggaggtgggagcaagaagaaaccactggatggagaatatttcacccttcagatccgtgggcgtgagcgcttcgagatgttccgagagctgaatgaggccttggaactcaaggatgcccaggctgggaaggagccaggggactacaaggacgacgacgacaagcaccaccaccatcaccactgataagcggccgc

SEQ ID NO: 130 - protein, 202 aa, Artificial Sequence
  Synthetic Construct
  VARIANT (199)..(200): Xaa = any amino acid
HVMELGLSWVVLAALLQGVQAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKGGGGSKKKPLDGEYFTLQIRGRERFEMFRELNEALELKDAQAGKEPGDYKDDDDKHHHHHHXXAA

SEQ ID NO: 131 - protein, 334 aa, Artificial Sequence
  Synthetic Construct
MGWSCIILFLVATATGVHSKQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 441 aa, Artificial Sequence
  Synthetic Construct
MGWSCIILFLVATATGVHSNAGVTQTPKFQVLKTGQSMTLQCAQDMNHEYMSWYRQDPGMGLRLIHYSVAIQTDQGEVPNGYNVSRSTIEDFPLRLLSAAPSQTSVYFCASSYLGNTGELFFGEGSRLTVLEDLNKVFPPEVAVFEPSEAEISHTQKATLVCLATGFFPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSGGGGSGGGGSLTDREWAEEWKHLDHLLNCIMDMVEKTRRSLTVLRRCQEADREELNYWIRRYSDAEHHHHHH

SEQ ID NO: 133 - protein, 334 aa, Artificial Sequence; identical to SEQ ID NO: 131
  Synthetic Construct

SEQ ID NO: 134 - protein, 456 aa, Artificial Sequence
  Synthetic Construct
MGWSCIILFLVATATGVHSNAGVTQTPKFQVLKTGQSMTLQCAQDMNHEYMSWYRQDPGMGLRLIHYSVAIQTDQGEVPNGYNVSRSTIEDFPLRLLSAAPSQTSVYFCASSYLGNTGELFFGEGSRLTVLEDLNKVFPPEVAVFEPSEAEISHTQKATLVCLATGFFPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGGGGSGGGGSGGGGSGGGGSLTDREWAEEWKHLDHLLNCIMDMVEKTRRSLTVLRRCQEADREELNYWIRRYSDAEHHHHHH

SEQ ID NO: 135 - protein, 334 aa, Artificial Sequence; identical to SEQ ID NO: 131
  Synthetic Construct

SEQ ID NO: 136 - protein, 594 aa, Artificial Sequence
  Synthetic Construct
MGWSCIILFLVATATGVHSNAGVTQTPKFQVLKTGQSMTLQCAQDMNHEYMSWYRQDPGMGLRLIHYSVAIQTDQGEVPNGYNVSRSTIEDFPLRLLSAAPSQTSVYFCASSYLGNTGELFFGEGSRLTVLEDLNKVFPPEVAVFEPSEAEISHTQKATLVCLATGFFPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSGGGGSGGGGSLTDREWAEEWKHLDHLLNCIMDMVEKTRRSLTVLRRCQEADREELNYWIRRYSDAEGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 137 - protein, 334 aa, Artificial Sequence; identical to SEQ ID NO: 131
  Synthetic Construct

SEQ ID NO: 138 - protein, 609 aa, Artificial Sequence
  Synthetic Construct
MGWSCIILFLVATATGVHSNAGVTQTPKFQVLKTGQSMTLQCAQDMNHEYMSWYRQDPGMGLRLIHYSVAIQTDQGEVPNGYNVSRSTIEDFPLRLLSAAPSQTSVYFCASSYLGNTGELFFGEGSRLTVLEDLNKVFPPEVAVFEPSEAEISHTQKATLVCLATGFFPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGGGGSGGGGSGGGGSGGGGSLTDREWAEEWKHLDHLLNCIMDMVEKTRRSLTVLRRCQEADREELNYWIRRYSDAEGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 139 - DNA, 912 bp, Artificial Sequence
  Synthetic Construct
atgggatggtcttgtataattctgttcctggtggcaacagcaacaggagtgcatagcgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtggcggcggaggcagcggaggcggaggttctggaggaggggggagtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaaaagaagaaaccactggatggagaatatttcacccttcagatccgtgggcgtgagcgcttcgagatgttccgagagctgaatgaggccttggaactcaaggatgcccaggctgggaaggagccaggg

SEQ ID NO: 140 - DNA, 801 bp, Artificial Sequence
  Synthetic Construct
atgggctggtcctgcatcatcctgtttctggtggccacagccacaggcgtgcacagcgatattgtgctgacacagagccccgccatcctgagtgcttctccaggcgagaaagtgaccatgacctgcagagccagcagcagcgtgaactacatggactggtatcagaagaagcccggcagcagccccaagccttggatctacgccacaagcaatctggccagcggagtgcctgccagattttctggctctggcagcggcacaagctacagcctgacaatcagcagagtggaagccgaggatgccgccacctactactgtcagcagtggtccttcaatcctcctaccttcggcggaggcaccaagctggaaatcaagggctctacatctggcggaggtggaagcggaggcggaggatctggtggtggtggatcttctgaggtccagctgcaacagtctggcgccgagcttgtgaaacctggcgcctctgtgaagatgagctgcaaggccagcggctacaccttcaccagctacaacatgcactgggtcaagcagacccctggacagggactcgagtggatcggagccatctatcccggcaatggcgacacctcctacaaccagaagttcaagggcaaagccacactgaccgccgacaagagcagcagcacagcctacatgcagctgagcagcctgaccagcgaggacagcgccgattactactgcgccagaagcaactactacggcagctcctactggttcttcgacgtgtggggagccggcaccacagtgacagtgtccagc

SEQ ID NO: 141 - DNA, 927 bp, Artificial Sequence
  Synthetic Construct
atgggctggtcctgcatcatcctgtttctggtggccacagccacaggcgtgcacagcgatattgtgctgacacagagccccgccatcctgagtgcttctccaggcgagaaagtgaccatgacctgcagagccagcagcagcgtgaactacatggactggtatcagaagaagcccggcagcagccccaagccttggatctacgccacaagcaatctggccagcggagtgcctgccagattttctggctctggcagcggcacaagctacagcctgacaatcagcagagtggaagccgaggatgccgccacctactactgtcagcagtggtccttcaatcctcctaccttcggcggaggcaccaagctggaaatcaagggctctacatctggcggaggtggaagcggaggcggaggatctggtggtggtggatcttctgaggtccagctgcaacagtctggcgccgagcttgtgaaacctggcgcctctgtgaagatgagctgcaaggccagcggctacaccttcaccagctacaacatgcactgggtcaagcagacccctggacagggactcgagtggatcggagccatctatcccggcaatggcgacacctcctacaaccagaagttcaagggcaaagccacactgaccgccgacaagagcagcagcacagcctacatgcagctgagcagcctgaccagcgaggacagcgccgattactactgcgccagaagcaactactacggcagctcctactggttcttcgacgtgtggggagccggcaccacagtgacagtgtccagcaagaaaaagcccctggacggcgagtacttcacactgcagatccggggcagagaacgcttcgagatgttcagagagctgaacgaggccctggaactgaaggatgcccaggccggaaaagagcccggc

SEQ ID NO: 142 - DNA, 1671 bp, Artificial Sequence
  Synthetic Construct
atgggctggtcctgcatcatcctgtttctggtggccacagccacaggcgtgcacagcgatattgtgctgacacagagccccgccatcctgagtgcttctccaggcgagaaagtgaccatgacctgcagagccagcagcagcgtgaactacatggactggtatcagaagaagcccggcagcagccccaagccttggatctacgccacaagcaatctggccagcggagtgcctgccagattttctggctctggcagcggcacaagctacagcctgacaatcagcagagtggaagccgaggatgccgccacctactactgtcagcagtggtccttcaatcctcctaccttcggcggaggcaccaagctggaaatcaagggctctacatctggcggaggtggaagcggaggcggaggatctggtggtggtggatcttctgaggtccagctgcaacagtctggcgccgagcttgtgaaacctggcgcctctgtgaagatgagctgcaaggccagcggctacaccttcaccagctacaacatgcactgggtcaagcagacccctggacagggactcgagtggatcggagccatctatcccggcaatggcgacacctcctacaaccagaagttcaagggcaaagccacactgaccgccgacaagagcagcagcacagcctacatgcagctgagcagcctgaccagcgaggacagcgccgattactactgcgccagaagcaactactacggcagctcctactggttcttcgacgtgtggggagccggcaccacagtgacagtgtccagcaagaaaaagcccctggacggcgagtacttcacactgcagatccggggcagagaacgcttcgagatgttcagagagctgaacgaggccctggaactgaaggatgcccaggccggaaaagagcccggcgatattgtgctgacacagagccccgccatcctgagtgcttctccaggcgagaaagtgaccatgacctgcagagccagcagcagcgtgaactacatggactggtatcagaagaagcccggcagcagccccaagccttggatctacgccacaagcaatctggccagcggagtgcctgccagattttctggctctggcagcggcacaagctacagcctgacaatcagcagagtggaagccgaggatgccgccacctactactgtcagcagtggtccttcaatcctcctaccttcggcggaggcaccaagctggaaatcaagggctctacatctggcggaggtggaagcggaggcggaggatctggtggtggtggatcttctgaggtccagctgcaacagtctggcgccgagcttgtgaaacctggcgcctctgtgaagatgagctgcaaggccagcggctacaccttcaccagctacaacatgcactgggtcaagcagacccctggacagggactcgagtggatcggagccatctatcccggcaatggcgacacctcctacaaccagaagttcaagggcaaagccacactgaccgccgacaagagcagcagcacagcctacatgcagctgagcagcctgaccagcgaggacagcgccgattactactgcgccagaagcaactactacggcagctcctactggttcttcgacgtgtggggagccggcaccacagtgacagtgtccagc

SEQ ID NO: 143 - DNA, 870 bp, Artificial Sequence
  Synthetic Construct
atgggatggtcttgtataattctgttcctggtggcaacagcaacaggagtgcatagcgaggtgcagctggttgaatctggcggaggactggttcagcctggcggatctctgagactgtcttgtgccgccagcggcttcaccttcagcgactactggatgtactgggtccgacaggcccctggcaaaggccttgaatgggtgtccgagatcaacaccaacggcctgatcacaaagtaccccgacagcgtgaagggcagattcaccatcagccgggacaacgccaagaacaccctgtacctgcagatgaacagcctgcggcctgaggataccgccgtgtactactgtgccagatctcccagcggattcaacagaggccagggcacactggtcaccgtgtcatctaagaagaaaccactggatggagaatatttcacccttcagatccgtgggcgtgagcgcttcgagatgttccgagagctgaatgaggccttggaactcaaggatgcccaggctgggaaggagccaggggaggtgcagctggttgaatctggcggaggactggttcaggctggcggatctctgagactgtcttgtgccgccagcggcagcagcttcagattcagagctatggcctggtacagacaggcccctgagaagcagagggatttcgtggccaccatcaacagcctgggcgagacaacatatgccaccgccgtggaaggccggttcaccatcagcagagacaacgccaagaacaccgtgtacctgcagatggacagcctgaagcctgaggataccgccgtgtactactgcaacgagcccagaggcaattactggggccagggcacacaagtgaccgtgtcatctcac

SEQ ID NO: 144 - DNA, 1653 bp, Artificial Sequence
  Synthetic Construct
atgggatggtcttgtataattctgttcctggtggcaacagcaacaggagtgcatagcgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtggcggcggaggcagcggaggcggaggttctggaggaggggggagtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaaaagaagaaaccactggatggagaatatttcacccttcagatccgtgggcgtgagcgcttcgagatgttccgagagctgaatgaggccttggaactcaaggatgcccaggctgggaaggagccagggcaggttcagctggttcagtctggcgccgaagtgaagaaacctggcagcagcgtgaaggtgtcctgcaaggccagcggctacagcttcaccgactaccacatccactgggtccgacaggctccaggacaaggcttggaatggatgggcgtgatcaaccctatgtacggcaccaccgattacaaccagcggttcaagggcagagtgaccatcaccgccgatgagagcacaagcaccgcctacatggaactgagcagcctgagaagcgaggacaccgccgtgtactactgcgccagatacgactactttaccggcaccggggtgtactggggacagggaacactggtcacagtgtcctctggcggcggaggcagcggaggcggaggttctggaggaggggggagtgacatcgtgatgacccagacacctctgagcctgagcgtgacacctggacagcctgccagcatcagctgcagatccagcagatctctggtgcacagccggggcaatacctacctgcactggtatctgcagaagcccggccagtctcctcagctgctgatctacaaggtgtccaaccggttcatcggcgtgcccgatagattttctggcagcggctctggcaccgacttcaccctgaagatctccagagtggaagccgaggacgtgggcgtgtactactgtagccagagcacccatctgcctttcacctttggccagggcaccaagctggaaatcaagcac

SEQ ID NO: 145 - DNA, 1254 bp, Artificial Sequence
  Synthetic Construct
atgggatggtcttgtataattctgttcctggtggcaacagcaacaggagtgcatagcgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtggcggcggaggcagcggaggcggaggttctggaggaggggggagtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaaaagaagaaaccactggatggagaatatttcacccttcagatccgtgggcgtgagcgcttcgagatgttccgagagctgaatgaggccttggaactcaaggatgcccaggctgggaaggagccaggggaggtgcagctggttgaatctggcggaggactggttcaggctggcggatctctgagactgtcttgtgccgccagcggcagcagcttcagattcagagctatggcctggtacagacaggcccctgagaagcagagggatttcgtggccaccatcaacagcctgggcgagacaacatatgccaccgccgtggaaggccggttcaccatcagcagagacaacgccaagaacaccgtgtacctgcagatggacagcctgaagcctgaggataccgccgtgtactactgcaacgagcccagaggcaattactggggccagggcacacaagtgaccgtgtcatctcac

SEQ ID NO: 146 - DNA, 525 bp, Artificial Sequence
  Synthetic Construct
atgggatggtcttgtataattctgttcctggtggcaacagcaacaggagtgcatagcgaggtgcagctggttgaatctggcggaggactggttcaggctggcggatctctgagactgtcttgtgccgccagcggcagcagcttcagattcagagctatggcctggtacagacaggcccctgagaagcagagggatttcgtggccaccatcaacagcctgggcgagacaacatatgccaccgccgtggaaggccggttcaccatcagcagagacaacgccaagaacaccgtgtacctgcagatggacagcctgaagcctgaggataccgccgtgtactactgcaacgagcccagaggcaattactggggccagggcacacaagtgaccgtgtcatctaagaagaaaccactggatggagaatatttcacccttcagatccgtgggcgtgagcgcttcgagatgttccgagagctgaatgaggccttggaactcaaggatgcccaggctgggaaggagccagggcac

SEQ ID NO: 147 - DNA, 960 bp, Artificial Sequence
  Synthetic Construct
atgggatggtcttgtataattctgttcctggtggcaacagcaacaggagtgcatagcgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtggcggcggaggcagcggaggcggaggttctggaggaggggggagtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaaggtggtggtggctccggaggcggcggctctggtggcggtggcagcaagaagaaaccactggatggagaatatttcacccttcagatccgtgggcgtgagcgcttcgagatgttccgagagctgaatgaggccttggaactcaaggatgcccaggctgggaaggagccagggcac

SEQ ID NO: 148 - DNA, 1623 bp, Artificial Sequence
  Synthetic Construct
atgggctggtcctgcatcatcctgtttctggtggccacagccacaggcgtgcacagcgatattgtgctgacacagagccccgccatcctgagtgcttctccaggcgagaaagtgaccatgacctgcagagccagcagcagcgtgaactacatggactggtatcagaagaagcccggcagcagccccaagccttggatctacgccacaagcaatctggccagcggagtgcctgccagattttctggctctggcagcggcacaagctacagcctgacaatcagcagagtggaagccgaggatgccgccacctactactgtcagcagtggtccttcaatcctcctaccttcggcggaggcaccaagctggaaatcaagggctctacatctggcggaggtggaagcggaggcggaggatctggtggtggtggatcttctgaggtccagctgcaacagtctggcgccgagcttgtgaaacctggcgcctctgtgaagatgagctgcaaggccagcggctacaccttcaccagctacaacatgcactgggtcaagcagacccctggacagggactcgagtggatcggagccatctatcccggcaatggcgacacctcctacaaccagaagttcaagggcaaagccacactgaccgccgacaagagcagcagcacagcctacatgcagctgagcagcctgaccagcgaggacagcgccgattactactgcgccagaagcaactactacggcagctcctactggttcttcgacgtgtggggagccggcaccacagtgacagtgtccagcggcggaggtggaagcggaggcggaggatctggtggtggtggatctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagaagaaaaagcccctggacggcgagtacttcacactgcagatccggggcagagaacgcttcgagatgttcagagagctgaacgaggccctggaactgaaggatgcccaggccggaaaagagcccggc

SEQ ID NO: 149 - DNA, 1578 bp, Artificial Sequence
  Synthetic Construct
atgggctggtcctgcatcatcctgtttctggtggccacagccacaggcgtgcacagcgatattgtgctgacacagagccccgccatcctgagtgcttctccaggcgagaaagtgaccatgacctgcagagccagcagcagcgtgaactacatggactggtatcagaagaagcccggcagcagccccaagccttggatctacgccacaagcaatctggccagcggagtgcctgccagattttctggctctggcagcggcacaagctacagcctgacaatcagcagagtggaagccgaggatgccgccacctactactgtcagcagtggtccttcaatcctcctaccttcggcggaggcaccaagctggaaatcaagggctctacatctggcggaggtggaagcggaggcggaggatctggtggtggtggatcttctgaggtccagctgcaacagtctggcgccgagcttgtgaaacctggcgcctctgtgaagatgagctgcaaggccagcggctacaccttcaccagctacaacatgcactgggtcaagcagacccctggacagggactcgagtggatcggagccatctatcccggcaatggcgacacctcctacaaccagaagttcaagggcaaagccacactgaccgccgacaagagcagcagcacagcctacatgcagctgagcagcctgaccagcgaggacagcgccgattactactgcgccagaagcaactactacggcagctcctactggttcttcgacgtgtggggagccggcaccacagtgacagtgtccagcaagaaaaagcccctggacggcgagtacttcacactgcagatccggggcagagaacgcttcgagatgttcagagagctgaacgaggccctggaactgaaggatgcccaggccggaaaagagcccggcgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 150 - DNA, 786 bp, Artificial Sequence
  Synthetic Construct
atgggatggtcttgtataattctgttcctggtggcaacagcaacaggagtgcatagcgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtggcggcggaggcagcggaggcggaggttctggaggaggggggagtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 151 - protein, 285 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKKKKPLDGEYFTLQIRGRERFEMFRELNEALELKDAQAGKEPG

SEQ ID NO: 152 - protein, 248 aa, Artificial Sequence
  Synthetic Construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGGSGGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 153 - protein, 290 aa, Artificial Sequence
  Synthetic Construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGGSGGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSKKKPLDGEYFTLQIRGRERFEMFRELNEALELKDAQAGKEPG

SEQ ID NO: 154 - protein, 538 aa, Artificial Sequence
  Synthetic Construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGGSGGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSKKKPLDGEYFTLQIRGRERFEMFRELNEALELKDAQAGKEPGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGGSGGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 155 - protein, 270 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSKKKPLDGEYFTLQIRGRERFEMFRELNEALELKDAQAGKEPGEVQLVESGGGLVQAGGSLRLSCAASGSSFRFRAMAWYRQAPEKQRDFVATINSLGETTYATAVEGRFTISRDNAKNTVYLQMDSLKPEDTAVYYCNEPRGNYWGQGTQVTVSS

SEQ ID NO: 156 - protein, 530 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKKKKPLDGEYFTLQIRGRERFEMFRELNEALELKDAQAGKEPGQVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSGGGGSGGGGSGGGGSDIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPTFGQGTKLEIK

SEQ ID NO: 157 - protein, 398 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKKKKPLDGEYFTLQIRGRERFEMFRELNEALELKDAQAGKEPGEVQLVESGGGLVQAGGSLRLSCAASGSSFRFRAMAWYRQAPEKQRDFVATINSLGETTYATAVEGRFTISRDNAKNTVYLQMDSLKPEDTAVYYCNEPRGNYWGQGTQVTVSS

SEQ ID NO: 158 - protein, 155 aa, Artificial Sequence
  Synthetic Construct
EVQLVESGGGLVQAGGSLRLSCAASGSSFRFRAMAWYRQAPEKQRDFVATINSLGETTYATAVEGRFTISRDNAKNTVYLQMDSLKPEDTAVYYCNEPRGNYWGQGTQVTVSSKKKPLDGEYFTLQIRGRERFEMFRELNEALELKDAQAGKEPG

SEQ ID NO: 159 - protein, 300 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGGSGGGGSGGGGSKKKPLDGEYFTLQIRGRERFEMFRELNEALELKDAQAGKEPG

SEQ ID NO: 160 - protein, 522 aa, Artificial Sequence
  Synthetic Construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGGSGGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKKKPLDGEYFTLQIRGRERFEMFRELNEALELKDAQAGKEPG

SEQ ID NO: 161 - protein, 507 aa, Artificial Sequence
  Synthetic Construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGGSGGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSKKKPLDGEYFTLQIRGRERFEMFRELNEALELKDAQAGKEPGAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 243 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIK

SEQ ID NO: 163 - protein, 10 aa, Homo sapiens
APELLGGPSV

SEQ ID NO: 164 - protein, 11 aa, Homo sapiens
PAPELLGGPSV

SEQ ID NO: 165 - protein, 9 aa, Homo sapiens
APPVAGPSV

SEQ ID NO: 166 - protein, 10 aa, Homo sapiens
PAPPVAGPSV

SEQ ID NO: 167 - protein, 21 aa, Homo sapiens
EPKSCDKTHTPAPELLGGPSV

SEQ ID NO: 168 - protein, 20 aa, Homo sapiens
EPKSCDKTHTAPELLGGPSV

SEQ ID NO: 169 - protein, 17 aa, Homo sapiens
ERKCCVEPAPPVAGPSV

SEQ ID NO: 170 - protein, 16 aa, Homo sapiens
ERKCCVEAPPVAGPSV

SEQ ID NO: 171 - protein, 23 aa, Homo sapiens
ELKTPLGDTTHTPAPELLGGPSV

SEQ ID NO: 172 - protein, 23 aa, Homo sapiens
ELKTPLGDTTHTPAPELLGGPSV

SEQ ID NO: 173 - protein, 21 aa, Homo sapiens
EPKSCDTPPPPAPELLGGPSV

SEQ ID NO: 174 - protein, 20 aa, Homo sapiens
EPKSCDTPPPAPELLGGPSV

SEQ ID NO: 175 - protein, 10 aa, Homo sapiens
APEFLGGPSV

SEQ ID NO: 176 - protein, 11 aa, Homo sapiens
PAPEFLGGPSV

SEQ ID NO: 177 - protein, 18 aa, Homo sapiens
ESKYGPPPAPEFLGGPSV

SEQ ID NO: 178 - protein, 17 aa, Homo sapiens
ESKYGPPAPEFLGGPSV

SEQ ID NO: 179 - protein, 214 aa, Artificial Sequence; identical to SEQ ID NO: 105
  Synthetic Construct

SEQ ID NO: 180 - protein, 4 aa, Homo sapiens
CPPC

SEQ ID NO: 181 - protein, 4 aa, Homo sapiens
CPRC

SEQ ID NO: 182 - protein, 4 aa, Homo sapiens
CPSC

SEQ ID NO: 183 - protein, 10 aa, Homo sapiens
EPKSCDKTHT

SEQ ID NO: 184 - protein, 7 aa, Homo sapiens
ERKCCVE

SEQ ID NO: 185 - protein, 12 aa, Homo sapiens
ELKTPLGDTTHT

SEQ ID NO: 186 - protein, 10 aa, Homo sapiens
EPKSCDTPPP

SEQ ID NO: 187 - protein, 7 aa, Homo sapiens
ESKYGPP

SEQ ID NO: 188 - DNA, 915 bp, Artificial Sequence
  Synthetic Construct
atgggttggagttgtataattctcttcctcgtcgctactgctactggtgttcattctgaggtccaattgttggagtccggcggcggcgaggtgcaaccaggtggttcactccggttgagttgcgccgcgtcaggcgggattttcgcgattaaaccaatatcatggtataggcaagcgcccgggaaacaacgcgaatgggtgtctactaccaccagttccggggcgactaactatgcggaatcagtaaaagggcgctttacaatatctcgcgataatgcgaagaatactttgtatttgcaaatgtcatctctcagggcggaagacactgctgtttattattgtaatgtctttgaatactggggtcaaggtacgttggtgactgttaagcccggtggcagtggcggctcagaggttcaactccttgaatccggaggaggtgaggtccaaccaggcggaagtctccgcctttcatgcgcagcgtccgggtttagtttctccattaacgcaatgggatggtatcgccaagcaccgggtaaaaggcgcgagttcgttgctgctattgaatcaggtaggaacacggtttacgctgaatccgtcaaagggcgatttacaatatcccgtgataatgcgaagaatacagtttatttgcaaatgagttcactcagggcagaagacacggcggtttattactgtggactgcttaaaggaaatcgggtcgtctccccctctgtcgcgtactggggacaaggaaccctcgtgaccgttaaacccaagaagaaacctctcgatggggagtacttcactctccaaattcgaggtagggagaggtttgaaatgtttagggaactcaatgaagctctcgagcttaaagacgcgcaagctggtaaggaaccagggcatcatcaccatcatcat

SEQ ID NO: 189 - DNA, 57 bp, Artificial Sequence
  Synthetic Construct
gcttactggggacaaggtacgctcgtaactgtcaaaccccatcatcaccatcatcat

SEQ ID NO: 190 - protein, 299 aa, Artificial Sequence
  Synthetic Construct
MGWSCIILFLVATATGVHSEVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQPGGSLRLSCAASGFSFSINAMGWYRQAPGKRREFVAAIESGRNTVYAESVKGRFTISRDNAKNTVYLQMSSLRAEDTAVYYCGLLKGNRVVSPSVAYWGQGTLVTVKPKKKPLDGEYFTLQIRGRERFEMFRELNEALELKDAQAGKEPG

SEQ ID NO: 191 - protein, 293 aa, Artificial Sequence
  Synthetic Construct
MGWSCIILFLVATATGVHSEVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPKKKPLDGEYFTLQIRGRERFEMFRELNEALELKDAQAGKEPGEVQLLESGGGEVQPGGSLRLSCAASGFSFSINAMGWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 192 - DNA, 1503 bp, Artificial Sequence
  Synthetic Construct
atgggctggtcatgtataatcctctttcttgtagccacagctaccggggtacactcagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtctcgagtgcctccactaaggggccgagtgtttttccacttgccccatccagtaagagcacctctggaggaactgccgccctgggttgccttgttaaggattacttccctgagccagtaactgttagctggaactctggcgctctgaccagcggagtgcacaccttccctgctgtgctgcagtcctcagggctgtactccctttctagtgtcgtaacagtgccatcttctagcctggggacccagacgtacatctgtaacgtgaatcataaacccagtaacacaaaggtagataagaaggttgaacctaagtcctgcgataagacacataccgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagaagaaaaagcccctggacggcgagtacttcacactgcagatccggggcagagaacgcttcgagatgttcagagagctgaacgaggccctggaactgaaggatgcccaggccggaaaagagcccggc

SEQ ID NO: 193 - DNA, 726 bp, Artificial Sequence
  Synthetic Construct
atgggctggtcatgtataatcctctttcttgtagccacagctaccggggtacactcagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtaagctcaaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 194 - DNA, 1575 bp, Artificial Sequence
  Synthetic Construct
atgggttggtcttgtattattcttttcctcgtcgcaaccgctaccggggtccattccgaggtccaattgcttgaatccggaggaggtgaagtgcaacccggtgggtcacttcggctctcctgcgccgcgagtggcgggattttcgctattaaaccaatttcctggtatcgtcaagcaccaggaaaacaacgagaatgggtgtcaactactacgtcttctggggcaactaactatgcagaatcagtcaaaggacgctttacgattagtcgagataatgcgaagaatactctttatctccaaatgtcatcactcagggcagaagacactgctgtctattattgtaatgttttcgaatactggggtcaaggaactttggtgaccgtaaagcccggcggcagcggcggtagtgaagtccaactcctcgagagtggaggaggggaagtgcaacccggcggaagtttgcggcttagttgcgcagcttccggttttagctttagtataaacgcaatgggatggtatcgccaagctccggggaaaaggcgagagttcgtagctgcaattgaatctggacgtaacacggtctacgcagaatccgtcaaagggcgttttactattagtcgcgataatgctaagaatacggtttatctccaaatgtcttcactccgggcagaagacacagctgtttattactgtggattgctcaaaggaaatcgggtcgtctcaccgtcagtcgcgtactggggacaaggaacccttgtgactgttaaaccaggtggtggtggggacaagacccacaccgcccctgaactgctgggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagccagagaacaactacaagacaacccctcctgtgctggacagcgacggctcattcttcctgtacagcaagctgacagtggacaagtccagatggcagcagggcaacgtgttctcctgctctgtgatgcacgaggccctgcacaaccactacacccagaagtccctgagcctgtctcctggcaaaaagaaaaagcccctggacggcgagtacttcacactgcaaatccggggcagagaacgcttcgagatgttcagagagctgaacgaggccctggaactgaaggatgcccaggccggaaaagagcccggc

SEQ ID NO: 195 - DNA, 1545 bp, Artificial Sequence
  Synthetic Construct
atgggttggtcttgtattattcttttcctcgtcgcaaccgctaccggggtccattccgaggtccaattgcttgaatccggaggaggtgaagtgcaacccggtgggtcacttcggctctcctgcgccgcgagtggcgggattttcgctattaaaccaatttcctggtatcgtcaagcaccaggaaaacaacgagaatgggtgtcaactactacgtcttctggggcaactaactatgcagaatcagtcaaaggacgctttacgattagtcgagataatgcgaagaatactctttatctccaaatgtcatcactcagggcagaagacactgctgtctattattgtaatgttttcgaatactggggtcaaggaactttggtgaccgtaaagcccggcggcagcggcggtagtgaagtccaactcctcgagtccggaggaggtgaagtgcaacccggtgggtcacttcggctctcctgcgccgcgagtggcgggattttcgctattaaaccaatttcctggtatcgtcaagcaccaggaaaacaacgagaatgggtgtcaactactacgtcttctggggcaactaactatgcagaatcagtcaaaggacgctttacgattagtcgagataatgcgaagaatactctttatctccaaatgtcatcactcagggcagaagacactgctgtctattattgtaatgttttcgaatactggggtcaaggaactttggtgaccgtaaagcccggtggtggtggggacaagacccacaccgcccctgaactgctgggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagccagagaacaactacaagacaacccctcctgtgctggacagcgacggctcattcttcctgtacagcaagctgacagtggacaagtccagatggcagcagggcaacgtgttctcctgctctgtgatgcacgaggccctgcacaaccactacacccagaagtccctgagcctgtctcctggcaaaaagaaaaagcccctggacggcgagtacttcacactgcaaatccggggcagagaacgcttcgagatgttcagagagctgaacgaggccctggaactgaaggatgcccaggccggaaaagagcccggc

SEQ ID NO: 196 - DNA, 1461 bp, Artificial Sequence
  Synthetic Construct
atgggctggtcatgtataatcctctttcttgtagccacagctaccggggtacactcaagcgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacaccgcccctgaactgctgggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttctcctgctctgtgatgcacgaggccctgcacaaccactacacccagaagtccctgagcctgtctcctggaaagaaaaagcccctggacggcgagtacttcacactgcaaatccggggcagagaacgcttcgagatgttcagagagctgaacgaggccctggaactgaaggatgcccaggccggaaaagagcccggc

SEQ ID NO: 197 - DNA, 405 bp, Artificial Sequence
  Synthetic Construct
atgggctggtcatgtataatcctctttcttgtagccacagctaccggggtacactcagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtaagctcacac

SEQ ID NO: 198 - DNA, 528 bp, Artificial Sequence
  Synthetic Construct
atgggctggtcatgtataatcctctttcttgtagccacagctaccggggtacactcagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtaagctcaaagaagaagccccttgacggcgagtacttcacactgcagatccggggcagagaacgcttcgagatgttcagagagctgaacgaggccctggaactgaaggatgcccaggccggaaaagagcccggc

SEQ ID NO: 199 - DNA, 876 bp, Artificial Sequence
  Synthetic Construct
atgggctggtcatgtataatcctctttcttgtagccacagctaccggggtacactcagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtaagctcaaagaagaagccccttgacggcgagtactttactttgcaaatacgaggcagagaaagatttgaaatgtttcgggaacttaacgaagcgctggagctgaaagacgcgcaagccggcaaagaacccggagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtaagctcacac

SEQ ID NO: 200 - DNA, 1248 bp, Artificial Sequence
  Synthetic Construct
atgggctggtcatgtataatcctctttcttgtagccacagctaccggggtacactcagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtctcgagtggcggcagcggcggtagtgaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtctcgtcaggcggaggcggttccggagggggaggatccgcctccactaaggggccgagtgtttttccacttgccccatccagtaagagcacctctggaggaactgccgccctgggttgccttgttaaggattacttccctgagccagtaactgttagctggaactctggcgctctgaccagcggagtgcacaccttccctgctgtgctgcagtcctcagggctgtactccctttctagtgtcgtaacagtgccatcttctagcctggggacccagacgtacatctgtaacgtgaatcataaacccagtaacacaaaggtagataagaaggttgaacctaagtcctgcgataagacacataccaagaagaaaccactggatggagaatatttcacccttcagatccgtgggcgtgagcgcttcgagatgttccgagagctgaatgaggccttggaactcaaggatgcccaggctgggaaggagccagggcac

SEQ ID NO: 201 - DNA, 750 bp, Artificial Sequence
  Synthetic Construct
atgggctggtcatgtataatcctctttcttgtagccacagctaccggggtacactcagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtctcgagtggcggaggcggttccggagggggaggatccggacagccaaaagcagccccatccgtaactctgttcccacctagttcagaggagcttcaagcaaacaaagccacacttgtttgccttattagtgatttttatcccggtgccgtgacagttgcctggaaagctgatagctcaccagtgaaagctggcgtggagacaaccacaccatctaaacaaagcaataacaagtatgctgccagctcatatctgagtctcactccagaacaatggaagtctcatcggtcctatagctgtcaagtgacccacgaaggcagtaccgtcgagaagaccgtggcaccaacagagtgtagc

SEQ ID NO: 202 - DNA, 756 bp, Artificial Sequence
  Synthetic Construct
atgggctggtcatgtataatcctctttcttgtagccacagctaccggggtacactcagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtctcgagtggcggaggcggttccggagggggaggatccaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcaatccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 203 - DNA, 1113 bp, Artificial Sequence
  Synthetic Construct
atgggctggtcatgtataatcctctttcttgtagccacagctaccggggtacactcagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtctcgagtggcggcagcggcggtagtgaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtctcgtcaggcggaggcggttccggagggggaggatccggacagccaaaagcagccccatccgtaactctgttcccacctagttcagaggagcttcaagcaaacaaagccacacttgtttgccttattagtgatttttatcccggtgccgtgacagttgcctggaaagctgatagctcaccagtgaaagctggcgtggagacaaccacaccatctaaacaaagcaataacaagtatgctgccagctcatatctgagtctcactccagaacaatggaagtctcatcggtcctatagctgtcaagtgacccacgaaggcagtaccgtcgagaagaccgtggcaccaacagagtgtagc

SEQ ID NO: 204 - DNA, 1119 bp, Artificial Sequence
  Synthetic Construct
atgggctggtcatgtataatcctctttcttgtagccacagctaccggggtacactcagaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtctcgagtggcggcagcggcggtagtgaagttcaactcgtcgagagtggtggcggcttggtccaaccaggtgggagtctccgccttagctgcgcagcaagtgggtttacgtttagtgattattggatgtattgggtgcggcaagctccgggaaagggactggagtgggtctctgaaattaacacgaatggtctcattaccaaatatcctgatagtgtaaaaggacgcttcacaatttctagggataatgcaaagaacactctctaccttcaaatgaattccctgcgtcccgaagacacggcggtttattattgcgctcgatcccctagtgggtttaatcgaggacaaggaacccttgtgacagtctcgtcaggcggaggcggttccggagggggaggatccaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcaatccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 205 - DNA, 1518 bp, Artificial Sequence
  Synthetic Construct
atgggctggtcatgtataatcctctttcttgtagccacagctaccggggtacactcagaagtgcagctgctggaatctggcggaggactggttcaacctggcggctctctgagactgtcttgtgccgccagcggcttcaccttcagcagctatatcatgatgtgggtccgacaggcccctggcaaaggccttgaatgggtgtccagcatctatcccagcggcggcatcaccttttacgccgacacagtgaagggcagattcaccatcagccgggacaacagcaagaacaccctgtacctgcagatgaacagcctgagagccgaggacaccgccgtgtactactgcgccagaatcaagctgggcaccgtgaccaccgtggattattggggacagggcaccctggtcaccgtctcgagtgcctccactaaggggccgagtgtttttccacttgccccatccagtaagagcacctctggaggaactgccgccctgggttgccttgttaaggattacttccctgagccagtaactgttagctggaactctggcgctctgaccagcggagtgcacaccttccctgctgtgctgcagtcctcagggctgtactccctttctagtgtcgtaacagtgccatcttctagcctggggacccagacgtacatctgtaacgtgaatcataaacccagtaacacaaaggtagataagaaggttgaacctaagtcctgcgataagacacataccgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagaagaaaaagcccctggacggcgagtacttcacactgcagatccggggcagagaacgcttcgagatgttcagagagctgaacgaggccctggaactgaaggatgcccaggccggaaaagagcccggc

SEQ ID NO: 206 - DNA, 705 bp, Artificial Sequence
  Synthetic Construct
atgggctggtcatgtataatcctctttcttgtagccacagctaccggggtacactcacagtctgctctgacacagcctgcctctgtgtctggctctcctggccagagcatcaccatcagctgtaccggcaccagctctgatgtcggcggctacaattacgtgtcctggtatcagcagcaccccggcaaggcccctaagctgatgatctacgacgtgtccaacagacccagcggcgtgtccaatagattctccggcagcaagagcggcaacaccgccagcctgacaattagcggactgcaggccgaggacgaggccgattactactgtagcagctacaccagctccagcaccagagtgtttggcaccggcacaaaagtgaccgtgctgggccagcctaaggccaatcctaccgtgacactgttccctccaagcagcgaggaactgcaggctaacaaggccacactcgtgtgcctgatcagcgacttttatcctggcgccgtgaccgtggcctggaaggctgatggatctcctgtgaaagccggcgtggaaaccaccaagcctagcaagcagagcaacaacaaatacgccgccagcagctacctgagcctgacacctgagcagtggaagtcccacagatcctacagctgccaagtgacccacgagggcagcaccgtggaaaaaacagtggcccctaccgagtgcagc

SEQ ID NO: 207 - DNA, 1521 bp, Artificial Sequence
  Synthetic Construct
atgggatggtcttgtataattctgttcctggtggcaacagcaacaggagtgcatagcgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcctccactaaggggccgagtgtttttccacttgccccatccagtaagagcacctctggaggaactgccgccctgggttgccttgttaaggattacttccctgagccagtaactgttagctggaactctggcgctctgaccagcggagtgcacaccttccctgctgtgctgcagtcctcagggctgtactccctttctagtgtcgtaacagtgccatcttctagcctggggacccagacgtacatctgtaacgtgaatcataaacccagtaacacaaaggtagataagaaggttgaacctaagtcctgcgataagacacataccgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagaagaaaaagcccctggacggcgagtacttcacactgcagatccggggcagagaacgcttcgagatgttcagagagctgaacgaggccctggaactgaaggatgcccaggccggaaaagagcccggc

SEQ ID NO: 208 - DNA, 699 bp, Artificial Sequence
  Synthetic Construct
atgggatggtcttgtataattctgttcctggtggcaacagcaacaggagtgcatagcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 209 - protein, 482 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKKKPLDGEYFTLQIRGRERFEMFRELNEALELKDAQAGKEPG

SEQ ID NO: 210 - protein, 223 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 506 aa, Artificial Sequence
  Synthetic Construct
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQPGGSLRLSCAASGFSFSINAMGWYRQAPGKRREFVAAIESGRNTVYAESVKGRFTISRDNAKNTVYLQMSSLRAEDTAVYYCGLLKGNRVVSPSVAYWGQGTLVTVKPGGGGDKTHTAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKKKKPLDGEYFTLQIRGRERFEMFRELNEALELKDAQAGKEPG

SEQ ID NO: 212 - protein, 496 aa, Artificial Sequence
  Synthetic Construct
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGGGDKTHTAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKKKKPLDGEYFTLQIRGRERFEMFRELNEALELKDAQAGKEPG

SEQ ID NO: 213 - protein, 468 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKKPLDGEYFTLQIRGRERFEMFRELNEALELKDAQAGKEPG

SEQ ID NO: 214 - protein, 115 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 215 - protein, 157 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSKKKPLDGEYFTLQIRGRERFEMFRELNEALELKDAQAGKEPG

SEQ ID NO: 216 - protein, 272 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSKKKPLDGEYFTLQIRGRERFEMFRELNEALELKDAQAGKEPGEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 217 - protein, 396 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGSGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTKKKPLDGEYFTLQIRGRERFEMFRELNEALELKDAQAGKEPG

SEQ ID NO: 218 - protein, 231 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGGSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 219 - protein, 233 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGGSKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 352 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGSGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGGSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 221 - protein, 354 aa, Artificial Sequence
  Synthetic Construct
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGSGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGGSKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 487 aa, Artificial Sequence
  Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKKKPLDGEYFTLQIRGRERFEMFRELNEALELKDAQAGKEPG

SEQ ID NO: 223 - protein, 216 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 224 - protein, 488 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKKKPLDGEYFTLQIRGRERFEMFRELNEALELKDAQAGKEPG

SEQ ID NO: 225 - protein, 39 aa, Homo sapiens
KKKPLDGEYFTLQIRGRERFEMFRELNEALELKDAQAGK

SEQ ID NO: 226 - protein, 38 aa, Mus musculus
KKKPLDGEYFTLKIRGRKRFEMFRELNEALELKDAHAT

SEQ ID NO: 227 - protein, 38 aa, Rattus rattus
KKKPLDGEYFTLKIRGRERFEMFRELNEALELKDARAA

SEQ ID NO: 228 - protein, 39 aa, Canis lupus familiaris
KKKPLDGEYFTLQIRGRERYEMFRNLNEALELKDAQSGK

SEQ ID NO: 229 - protein, 39 aa, Felis catus
KKKPLDGEYFTLQIRGRERFEMFRELNEALELKDAQSGK

SEQ ID NO: 230 - protein, 39 aa, Equus caballus
KKKPLDGEYFTLQIRGRERFEMFRELNEALELKDAQTGK

SEQ ID NO: 231 - protein, 6 aa, Artificial Sequence
  Synthetic Construct
HHHHHH

SEQ ID NO: 232 - protein, 8 aa, Artificial Sequence
  Synthetic Construct
DYKDDDDK

SEQ ID NO: 233 - protein, 14 aa, Artificial Sequence
  Synthetic Construct
DYKDDDDKHHHHHH

SEQ ID NO: 234 - protein, 19 aa, Artificial Sequence
  Synthetic Construct
  VARIANT (15)..(16): Xaa = Any Amino Acid
DYKDDDDKHHHHHHXXAAA

SEQ ID NO: 235 - protein, 4 aa, Artificial Sequence
  Synthetic Construct
  VARIANT 2, 3: Xaa = Any Amino Acid
CXXC

SEQ ID NO: 236 - protein, 50 aa, Artificial Sequence
  Synthetic Construct
  VARIANT (6)..(10), (11)..(15), (16)..(20), (21)..(25),(26)..(30), (31)..(35), (36)..(40), (41)..(45), (46)..(50): Can be present or absent
GGGGSGGGGSGGGGSGGGGSGGGGSGGGGSGGGGSGGGGSGGGGSGGGGS

SEQ ID NO: 237 - protein, 9 aa, Artificial Sequence
  Synthetic Construct
SLLMWITQC

SEQ ID NO: 238 - protein, 5 aa, Artificial Sequence
  Synthetic Construct
CPPCP

SEQ ID NO: 239 - protein, 5 aa, Artificial Sequence
  Synthetic Construct
CPRCP

SEQ ID NO: 240 - protein, 5 aa, Artificial Sequence
  Synthetic Construct
CPSCP